(19) United States
(12) Patent Application Publication
(10) Pub. No.: US 2005/0186573 A1
(43) Pub. Date: Aug. 25, 2005

(54) Polynucleotides for use as tags and tag complements in the detection of nucleic acid sequences

(76) Inventor: Richard Adam Janeczko, Oakville (CA)

Correspondence Address: BLAKE, CASSELS & GRAYDON LLP, BOX 25, COMMERCE COURT WEST, 199 BAY STREET, SUITE 2800, TORONTO, ON, M5L 1A9, CA

(21) Appl. No.: 10/625,755

(22) Filed: Jul. 24, 2003

Related U.S. Application Data
(60) Provisional application No. 60/398,037, filed on Jul. 24, 2002.

(51) Int. Cl. C12Q 1/68; C07H 21/02; C12P 19/34

(52) U.S. Cl. 435/6; 435/91.2; 536/23.1

(57) ABSTRACT

A family of minimally cross-hybridizing nucleotide sequences and their use in the detection of nucleic acid sequences is described. Specifically described is the use of two distinct families of 210 and 1168 24mers in the detection of nucleic acid sequences.

### FIELD OF THE INVENTION

This invention relates to the use of families of oligonucleotides for use as tags, for example, in the sorting of molecules, identification of target nucleic acid molecules or for analyzing the presence of a mutation or polymorphism at a locus of each target nucleic acid molecule.

### BACKGROUND OF THE INVENTION

Single-nucleotide polymorphisms (SNPs) are the most common form of genetic polymorphism. This, coupled with their potential as functional variants, has produced a great deal of interest in SNPs both as pharmacogenetic indicators and as markers for mapping genes for complex diseases. A large number of SNPs have already been identified with >21,000 entries on the NCBI's SNP database alone. Many recent studies have focused on identifying polymorphisms that lie in the coding sequence of potential candidate genes for common diseases. The ability to genotype this abundant source of variation rapidly and accurately is becoming an ever more important goal in the genetics community. A variety of technologies have the potential to transfer to high-throughput genotyping laboratories. These include 5′ exonuclease assays, such as TaqMan (Livak et al. 1995), molecular beacons (Tyagi et al. 1996), dye-labeled oligonucleotide ligation (DOL) (Chen et al. 1998), oligonucleotide-ligation assays (OLAs) (To be et al. 1996), minisequencing (Chen and Kwok 1997; Pastinen et al. 1997), microarray technology (Hacia et al. 1998; Wang et al. 1998), mass spectroscopy (Ross et al. 1998) and the scorpions assay (Whitcombe et al. 1999). However, no single chemistry has gained acceptance as the technology of choice. A suitable method for such applications must be accurate and homogenous, develop a robust, easily interpretable signal, and be flexible enough to extend to novel foci with little optimization. These features will lend the technology to automation.

Third Wave Technologies, Inc., has developed a new mutation detection method referred to as the Invader Assay. The Invader Assay is based on a novel linear signal amplification technology that requires specific hybridization of two “overlapping” oligonucleotides and subsequent recognition and cleavage of this structure by the Cleavase enzyme. Cleavases are bacterial enzymes that cleave unpaired DNA strands or “flaps” near a nick, for instance when the 5′ end of a sequence is displaced by the 3′ end of an elongating upstream oligonucleotide. Enzymes with this so-called flap endonuclease activity typically excise the redundant 5′ “flap” of the downstream oligonucleotide, leaving a simple nick to be repaired by lipases. The excised “flap” is subsequently detected by one of several methods commonly known in the art. Cleavases have stringent requirements relative to the structure formed by such overlapping DNA sequences, and can be used to specifically detect single base pair mismatches immediately upstream of the cleavage site on the downstream DNA strand. Thermostable cleavages permit reactions to be performed at temperatures sufficiently high to promote turnover and consequent signal amplification without the need for temperature cycling.

While the Invader Assay offers exquisite specificity, its use in the detection of multiple distinct target nucleic acids in a single experiment i.e., multiplexing, is limited. This is because if the Invader Assay is to be used in a high-throughput gene microarray format, the most efficient method of detecting the excised “flap” sequence is to capture the sequence by hybridization to its complementary nucleic acid sequence attached to a solid phase support.

Working in a highly parallel hybridization environment requiring specific hybridization imposes very rigorous selection criteria for the design of families of oligonucleotides that are to be used. The success of these approaches is dependent on the specific hybridization of a probe and its complement. Problems arise as the family of nucleic acid molecules cross-hybridise or hybridise incorrectly to the target sequences. While it is common to obtain incorrect hybridization resulting in false positives or an inability to form hybrids resulting in false negatives, the frequency of such results must be minimized. In order to achieve this goal certain thermodynamic properties of forming nucleic acid hybrids must be considered. The temperature at which oligonucleotides form duplexes with their complementary sequences known as the T. (the temperature at which 50% of the nucleic acid duplex is dissociated) varies according to a number of sequence dependent properties including the hydrogen bonding energies of the canonical pairs A-T and G-C (reflected in GC or base composition), stacking free energy and, to a lesser extent, nearest neighbour interactions. These energies vary widely among oligonucleotides that are typically used in hybridization assays. For example, hybridization of two probe sequences composed of 24 nucleotides, one with a 40% GC content and the other with a 60% GC content, with its complementary target under standard conditions theoretically may have a 10° C. difference in melting temperature (Mueller et al., Current Protocols in Mol. Biol.; 15, 5:1993). Problems in hybridization occur when the hybrids are allowed to form under hybridization conditions that include a single hybridization temperature that is not optimal for correct hybridization of all oligonucleotide sequences of a set. Mismatch hybridization of non-complementary probes can occur forming duplexes with measurable mismatch stability (Santalucia et al., Biochemistry; 38: 3468-77, 1999). Mismatching of duplexes in a particular set of oligonucleotides can occur under hybridization conditions where the mismatch results in a decrease in duplex stability that results in a higher Tₘ than the least stable correct duplex of that particular set. For example, if hybridization is carried out under conditions that favor the AT-rich perfect match duplex sequence, the possibility exists for hybridizing a GC-rich duplex sequence that contains a mismatched base having a melting temperature that is still above the correctly formed AT-rich duplex. Therefore, design of families of oligonucleotide sequences that can be used in multiplexed hybridization reactions must include consideration for the thermodynamic properties of oligonucleotides and duplex formation that will reduce or eliminate cross hybridization behavior within the designed oligonucleotide set.

The development of such families of tags has been attempted over the years with varying degrees of success. There are a number of different approaches for selecting sequences for use in multiplexed hybridization assays. The selection of sequences that can be used as zipcodes or tags in an addressable array has been described in the patent literature in an approach taken by Brenner and co-workers. U.S. Pat. No. 5,654,413 describes a population of oligonucleotide tags (and corresponding tag complements) in which each oligonucleotide tag includes a plurality of subunits, each subunit consisting of an oligonucleotide having a length of from three to six nucleotides and each subunit being selected from a minimally cross hybridizing set, wherein a subunit of the set would have at least two mismatches with any other sequence of the set. Table II of the Brenner patent specification describes exemplary groups of 4mer subunits that are minimally cross hybridizing according to the aforementioned criteria. In the approach taken by Brenner, constructing non cross-hybridizing oligonucleotides, relies on the use of subunits that form a duplex having at least two mismatches with the complement of any other subunit of the same set. The ordering of subunits in the construction of oligonucleotide tags is not specifically defined.

Parameters used in the design of tags based on subunits are discussed in Barany et al. (WO 9731256). For example, in the design of polynucleotide sequences that are for example 24 nucleotides in length (24mer) derived from a set of four possible tetramers in which each 24mer “address” differs from its nearest 24mer neighbour by 3 tetramers. They discuss further that, if each tetramer differs from each other by at least two nucleotides, then each 24mer will differ from the next by at least six nucleotides. This is determined without consideration for insertions or deletions when forming the alignment between any two sequences of the set. In this way a unique “zip code” sequence is generated. The zip code is ligated to a label in a target dependent manner, resulting in a unique “zip code” which is then allowed to hybridise to its address on the chip. To minimise cross-hybridisation of a “zip code” to other “addresses”, the hybridization reaction is carried out at temperatures of 75-80° C. Due to the high temperature conditions for hybridization, 24mers that have partial homology hybridise to a lesser extent than sequences with perfect complementarity and represent ‘dead zones’. This approach of implementing stringent hybridization conditions for example, involving high temperature hybridization, is also practiced by Brenner et. al.

The current state of technology for designing non-cross hybridizing tags based on subunits does not provide sufficient guidance to construct a family of relatively large numbers of sequences with practical value in assays that require stringent non-cross hybridizing behavior.

Thus, while it is desirable to have, at once in a gene microarray format, a large number of “flap” molecules incorporated into the Invader Assay, the “flap” molecules should each be highly selective for its own complement sequence. While such an array provides the advantage that the family of molecules making up the grid is entirely of design, and does not rely on sequences as they occur in nature, the provision of a family of molecules, which is, sufficiently large and where each individual member is sufficiently selective for its complement over all the other zipcode molecules (i.e., where there is sufficiently low cross-hybridization, or cross-talk) continues to elude researchers.

### SUMMARY OF THE INVENTION

The present invention relates to the use of one set of 210 and a second set of 1168 minimally cross-hybridizing oligonucleotide sequences for use in the Invader Assay. The incorporation of these sequences into one of the two probes, and subsequent structure dependent cleavage of the minimally cross-hybridizing sequences upon hybridization to the target nucleic acid molecule enables the Invader Assay to be used in the analysis of multiple gene n a gene microarray.

Using the method of Benight et al. a family of 100 sequences was obtained using a computer algorithm to have optimal hybridization properties for use in nucleic acid detection assays. The sequence set of 100 oligonucleotides was characterized in hybridization assays, demonstrating the ability of family members to correctly hybridize to their complementary sequences with an absence of cross hybridization. These are the sequences having SEQ ID NOs:1 to 100 of Table I. This set of sequences has been expanded to include an additional 110 sequences that can be grouped with the original 100 sequences as having non-cross hybridizing properties, based on the characteristics of the original set of 100 sequences. These additional sequences are identified as SEQ ID NOs:101 to 210 of the sequences in Table I. How these sequences were obtained is described below.

Variant families of sequences (seen as tags or tag complements) of a family of sequences taken from Table I are also part of the invention. For the purposes of discussion, families of tag complements will be described.

A family of complements is obtained from a set of oligonucleotides based on a family of oligonucleotides such as those of Table I. For illustrative purposes, providing a family of complements based on the oligonucleotides of Table I will be described.

Firstly, the groups of sequences based on the oligonucleotides of Table I can be represented as follows:

TABLE IA
Numeric sequences corresponding to word
patterns of a set of oligonucleotides

| | |
|---|---|
| Sequence | |
| Identifier | Numeric Pattern |

| | | | | | | |
|---|---|---|---|---|---|---|
| 1 | 1 | 4 | 6 | 6 | 1 | 3 |
| 2 | 2 | 4 | 5 | 5 | 2 | 3 |
| 3 | 1 | 8 | 1 | 2 | 3 | 4 |
| 4 | 1 | 7 | 1 | 9 | 8 | 4 |
| 5 | 1 | 1 | 9 | 2 | 6 | 9 |
| 6 | 1 | 2 | 4 | 3 | 9 | 6 |
| 7 | 9 | 8 | 9 | 8 | 10 | 9 |
| 8 | 9 | 1 | 2 | 3 | 8 | 10 |
| 9 | 8 | 8 | 7 | 4 | 3 | 1 |
| 10 | 1 | 1 | 1 | 1 | 1 | 2 |
| 11 | 2 | 1 | 3 | 3 | 2 | 2 |
| 12 | 3 | 1 | 2 | 2 | 3 | 2 |
| 13 | 4 | 1 | 4 | 4 | 4 | 2 |
| 14 | 1 | 2 | 3 | 3 | 1 | 1 |
| 15 | 1 | 3 | 2 | 2 | 1 | 4 |
| 16 | 3 | 3 | 3 | 3 | 3 | 4 |
| 17 | 4 | 3 | 1 | 1 | 4 | 4 |
| 18 | 3 | 4 | 1 | 1 | 3 | 3 |
| 19 | 3 | 6 | 6 | 6 | 3 | 5 |
| 20 | 6 | 6 | 1 | 1 | 6 | 5 |
| 21 | 7 | 6 | 7 | 7 | 7 | 5 |
| 22 | 8 | 7 | 5 | 5 | 8 | 8 |
| 23 | 2 | 1 | 7 | 7 | 1 | 1 |
| 24 | 2 | 3 | 2 | 3 | 1 | 3 |
| 25 | 2 | 6 | 5 | 6 | 1 | 6 |
| 26 | 4 | 8 | 1 | 1 | 3 | 8 |
| 27 | 5 | 3 | 1 | 1 | 6 | 3 |
| 28 | 5 | 6 | 8 | 8 | 6 | 6 |
| 29 | 8 | 3 | 6 | 5 | 7 | 3 |
| 30 | 1 | 2 | 3 | 1 | 4 | 6 |
| 31 | 1 | 5 | 7 | 5 | 4 | 3 |
| 32 | 2 | 1 | 6 | 7 | 3 | 6 |
| 33 | 2 | 6 | 1 | 3 | 3 | 1 |
| 34 | 2 | 7 | 6 | 8 | 3 | 1 |
| 35 | 3 | 4 | 3 | 1 | 2 | 5 |
| 36 | 3 | 5 | 6 | 1 | 2 | 7 |
| 37 | 3 | 6 | 1 | 7 | 2 | 7 |
| 38 | 4 | 6 | 3 | 5 | 1 | 7 |
| 39 | 5 | 4 | 6 | 3 | 8 | 6 |
| 40 | 6 | 8 | 2 | 3 | 7 | 1 |
| 41 | 7 | 1 | 7 | 8 | 6 | 3 |
| 42 | 7 | 3 | 4 | 1 | 6 | 8 |
| 43 | 4 | 7 | 7 | 1 | 2 | 4 |
| 44 | 3 | 6 | 5 | 2 | 6 | 3 |
| 45 | 1 | 4 | 1 | 4 | 6 | 1 |
| 46 | 3 | 3 | 1 | 4 | 8 | 1 |
| 47 | 8 | 3 | 3 | 5 | 3 | 8 |
| 48 | 1 | 3 | 6 | 6 | 3 | 7 |
| 49 | 7 | 3 | 8 | 6 | 4 | 7 |
| 50 | 3 | 1 | 3 | 7 | 8 | 6 |
| 51 | 10 | 9 | 5 | 5 | 10 | 10 |
| 52 | 7 | 10 | 10 | 10 | 7 | 9 |
| 53 | 9 | 9 | 7 | 7 | 10 | 9 |
| 54 | 9 | 3 | 10 | 3 | 10 | 3 |
| 55 | 9 | 6 | 3 | 4 | 10 | 6 |
| 56 | 10 | 4 | 10 | 3 | 9 | 4 |
| 57 | 3 | 9 | 3 | 10 | 4 | 9 |
| 58 | 9 | 10 | 5 | 9 | 4 | 8 |
| 59 | 3 | 9 | 4 | 9 | 10 | 7 |
| 60 | 3 | 5 | 9 | 4 | 10 | 8 |
| 61 | 4 | 10 | 5 | 4 | 9 | 3 |
| 62 | 5 | 3 | 3 | 9 | 8 | 10 |
| 63 | 6 | 8 | 6 | 9 | 7 | 10 |
| 64 | 4 | 6 | 10 | 9 | 6 | 4 |
| 65 | 4 | 9 | 8 | 10 | 8 | 3 |
| 66 | 7 | 7 | 9 | 10 | 5 | 3 |
| 67 | 8 | 8 | 9 | 3 | 9 | 10 |
| 68 | 8 | 10 | 2 | 9 | 5 | 9 |
| 69 | 9 | 6 | 2 | 2 | 7 | 10 |
| 70 | 9 | 7 | 5 | 3 | 10 | 6 |
| 71 | 10 | 3 | 6 | 8 | 9 | 2 |
| 72 | 10 | 9 | 3 | 2 | 7 | 3 |
| 73 | 8 | 9 | 10 | 3 | 6 | 2 |
| 74 | 3 | 2 | 5 | 10 | 8 | 9 |
| 75 | 8 | 2 | 3 | 10 | 2 | 9 |
| 76 | 6 | 3 | 9 | 8 | 2 | 10 |
| 77 | 3 | 7 | 3 | 9 | 9 | 10 |
| 78 | 9 | 10 | 1 | 1 | 9 | 4 |
| 79 | 10 | 1 | 9 | 1 | 4 | 1 |
| 80 | 7 | 1 | 10 | 9 | 8 | 1 |
| 81 | 9 | 1 | 10 | 1 | 10 | 6 |
| 82 | 9 | 6 | 9 | 1 | 3 | 10 |
| 83 | 3 | 10 | 8 | 8 | 9 | 1 |
| 84 | 3 | 8 | 1 | 9 | 10 | 3 |
| 85 | 9 | 10 | 1 | 3 | 6 | 9 |
| 86 | 1 | 9 | 1 | 10 | 3 | 1 |
| 87 | 1 | 4 | 9 | 6 | 8 | 10 |
| 88 | 3 | 3 | 9 | 6 | 1 | 10 |
| 89 | 5 | 3 | 1 | 6 | 9 | 10 |
| 90 | 6 | 1 | 8 | 10 | 9 | 6 |
| 91 | 5 | 9 | 9 | 4 | 10 | 3 |
| 92 | 2 | 10 | 9 | 1 | 9 | 5 |
| 93 | 10 | 10 | 7 | 2 | 1 | 9 |
| 94 | 10 | 9 | 9 | 1 | 8 | 2 |
| 95 | 1 | 8 | 6 | 8 | 9 | 10 |
| 96 | 1 | 9 | 1 | 3 | 8 | 10 |
| 97 | 9 | 6 | 9 | 10 | 1 | 2 |
| 98 | 1 | 10 | 8 | 9 | 9 | 2 |
| 99 | 1 | 9 | 6 | 7 | 2 | 9 |
| 100 | 4 | 3 | 9 | 3 | 5 | 1 |
| 101 | 5 | 11 | 10 | 14 | 12 | 1 |
| 102 | 7 | 12 | 4 | 13 | 3 | 2 |
| 103 | 5 | 5 | 4 | 4 | 12 | 9 |
| 104 | 2 | 13 | 13 | 11 | 13 | 13 |
| 105 | 10 | 2 | 5 | 4 | 12 | 7 |
| 106 | 11 | 7 | 4 | 11 | 6 | 4 |
| 107 | 12 | 12 | 1 | 9 | 11 | 11 |
| 108 | 12 | 9 | 4 | 14 | 12 | 6 |
| 109 | 12 | 7 | 13 | 2 | 9 | 11 |
| 110 | 9 | 11 | 3 | 4 | 1 | 3 |
| 111 | 10 | 5 | 12 | 11 | 4 | 4 |
| 112 | 4 | 13 | 7 | 12 | 1 | 5 |
| 113 | 9 | 13 | 10 | 11 | 11 | 6 |
| 114 | 10 | 14 | 14 | 10 | 1 | 3 |
| 115 | 2 | 14 | 1 | 10 | 4 | 5 |
| 116 | 10 | 12 | 12 | 7 | 11 | 10 |
| 117 | 9 | 11 | 2 | 12 | 8 | 11 |
| 118 | 2 | 8 | 5 | 2 | 12 | 14 |
| 119 | 1 | 8 | 13 | 3 | 7 | 8 |
| 120 | 9 | 4 | 7 | 5 | 4 | 2 |
| 121 | 13 | 2 | 12 | 7 | 1 | 12 |
| 122 | 11 | 10 | 9 | 7 | 5 | 11 |
| 123 | 8 | 12 | 2 | 2 | 12 | 7 |
| 124 | 5 | 2 | 14 | 3 | 4 | 13 |
| 125 | 1 | 8 | 8 | 1 | 5 | 9 |
| 126 | 14 | 5 | 11 | 10 | 13 | 3 |
| 127 | 14 | 1 | 4 | 13 | 2 | 4 |
| 128 | 4 | 4 | 5 | 11 | 3 | 10 |
| 129 | 10 | 9 | 2 | 3 | 3 | 11 |
| 130 | 11 | 4 | 8 | 14 | 3 | 4 |
| 131 | 5 | 1 | 14 | 8 | 11 | 2 |
| 132 | 14 | 3 | 11 | 6 | 12 | 5 |
| 133 | 13 | 4 | 4 | 1 | 10 | 1 |
| 134 | 6 | 10 | 11 | 6 | 5 | 1 |
| 135 | 5 | 8 | 12 | 5 | 1 | 7 |
| 136 | 4 | 5 | 9 | 6 | 9 | 2 |
| 137 | 13 | 2 | 4 | 4 | 2 | 3 |
| 138 | 11 | 2 | 2 | 5 | 9 | 3 |
| 139 | 8 | 1 | 10 | 12 | 2 | 8 |
| 140 | 12 | 7 | 9 | 11 | 4 | 1 |
| 141 | 12 | 1 | 4 | 14 | 3 | 13 |
| 142 | 11 | 2 | 7 | 10 | 4 | 1 |
| 143 | 3 | 4 | 12 | 11 | 11 | 11 |
| 144 | 3 | 3 | 4 | 2 | 12 | 11 |
| 145 | 1 | 5 | 9 | 4 | 2 | 1 |
| 146 | 6 | 1 | 12 | 2 | 10 | 5 |
| 147 | 10 | 5 | 1 | 12 | 2 | 14 |
| 148 | 2 | 11 | 7 | 9 | 4 | 11 |
| 149 | 7 | 4 | 4 | 5 | 14 | 12 |
| 150 | 12 | 5 | 2 | 1 | 10 | 12 |
| 151 | 5 | 9 | 2 | 11 | 6 | 1 |
| 152 | 12 | 14 | 3 | 6 | 1 | 14 |
| 153 | 5 | 9 | 11 | 10 | 1 | 4 |
| 154 | 2 | 5 | 12 | 14 | 10 | 10 |
| 155 | 4 | 5 | 8 | 4 | 5 | 6 |
| 156 | 10 | 12 | 4 | 6 | 12 | 5 |
| 157 | 4 | 2 | 1 | 13 | 6 | 8 |
| 158 | 9 | 10 | 10 | 14 | 5 | 3 |
| 159 | 6 | 14 | 10 | 11 | 3 | 3 |
| 160 | 2 | 9 | 10 | 12 | 5 | 7 |
| 161 | 13 | 3 | 7 | 10 | 5 | 12 |
| 162 | 6 | 4 | 1 | 2 | 5 | 13 |
| 163 | 6 | 1 | 13 | 4 | 14 | 13 |
| 164 | 2 | 12 | 1 | 14 | 1 | 9 |
| 165 | 4 | 11 | 13 | 2 | 6 | 10 |
| 166 | 1 | 10 | 7 | 4 | 5 | 8 |
| 167 | 7 | 2 | 2 | 10 | 13 | 4 |
| 168 | 8 | 2 | 11 | 4 | 6 | 14 |
| 169 | 4 | 8 | 2 | 6 | 2 | 3 |
| 170 | 7 | 1 | 12 | 11 | 2 | 9 |
| 171 | 5 | 6 | 10 | 4 | 13 | 4 |
| 172 | 5 | 10 | 4 | 11 | 9 | 3 |
| 173 | 3 | 11 | 9 | 3 | 2 | 3 |
| 174 | 8 | 15 | 6 | 20 | 17 | 19 |
| 175 | 21 | 10 | 15 | 3 | 7 | 11 |
| 176 | 11 | 7 | 17 | 20 | 14 | 9 |
| 177 | 16 | 6 | 17 | 13 | 21 | 21 |
| 178 | 10 | 15 | 22 | 6 | 17 | 21 |
| 179 | 15 | 7 | 17 | 10 | 22 | 22 |
| 180 | 3 | 20 | 8 | 15 | 20 | 16 |
| 181 | 17 | 21 | 10 | 16 | 6 | 22 |
| 182 | 6 | 21 | 14 | 14 | 14 | 16 |
| 183 | 7 | 17 | 3 | 20 | 10 | 7 |
| 184 | 16 | 19 | 14 | 17 | 7 | 21 |
| 185 | 20 | 16 | 7 | 15 | 22 | 10 |
| 186 | 20 | 10 | 18 | 11 | 22 | 18 |
| 187 | 18 | 7 | 19 | 15 | 7 | 22 |
| 188 | 21 | 18 | 7 | 21 | 16 | 3 |
| 189 | 14 | 13 | 7 | 22 | 17 | 13 |
| 190 | 19 | 7 | 8 | 12 | 10 | 17 |
| 191 | 15 | 3 | 21 | 14 | 9 | 7 |
| 192 | 19 | 6 | 15 | 7 | 14 | 14 |
| 193 | 4 | 17 | 10 | 15 | 20 | 19 |
| 194 | 21 | 6 | 18 | 4 | 20 | 16 |
| 195 | 2 | 19 | 8 | 17 | 6 | 13 |
| 196 | 12 | 12 | 6 | 17 | 4 | 20 |
| 197 | 16 | 21 | 12 | 10 | 19 | 16 |
| 198 | 14 | 14 | 15 | 2 | 7 | 21 |
| 199 | 8 | 16 | 21 | 6 | 22 | 16 |
| 200 | 14 | 17 | 22 | 14 | 17 | 20 |
| 201 | 10 | 21 | 7 | 15 | 21 | 18 |
| 202 | 16 | 13 | 20 | 18 | 21 | 12 |
| 203 | 15 | 7 | 4 | 22 | 14 | 13 |
| 204 | 7 | 19 | 14 | 8 | 15 | 4 |
| 205 | 4 | 5 | 3 | 20 | 7 | 16 |
| 206 | 22 | 18 | 6 | 18 | 13 | 20 |
| 207 | 19 | 6 | 16 | 3 | 13 | 3 |
| 208 | 18 | 6 | 22 | 7 | 20 | 18 |
| 209 | 10 | 17 | 11 | 21 | 8 | 13 |
| 210 | 7 | 10 | 17 | 19 | 10 | 14 |

Here, each of the numerals 1 to 22 (numeric identifiers) represents a 4mer and the pattern of numerals 1 to 10 of the sequences in the above list corresponds to the pattern of tetrameric oligonucleotide segments present in the oligonucleotides of Table I, which oligonucleotides have been found to be non-cross-hybridizing, as described further in the detailed examples. Each 4mer is selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY. Here W, X and Y represent nucleotide bases, A, G, C, etc., the assignment of bases being made according to rules described below.

Given this numeric pattern, a 4mer is assigned to a numeral. For example, 1=WXYY, 2=YWXY, etc. Once a given 4mer has been assigned to a given numeral, it is not assigned for use in the position of a different numeral. It is possible, however, to assign a different 4mer to the same numeral. That is, for example, the numeral 1 in one position could be assigned WXYY and another numeral 1, in a different position, could be assigned XXXW, but none of the other numerals 2 to 10 can then be assigned WXYY or XXXW. A different way of saying this is that each of 1 to 10 is assigned a 4mer from the list of eighty-one 4mers indicated so as to be different from all of the others of 1 to 10.

In the case of the specific oligonucleotides given in Table I, 1=WXYY, 2=YWXY, 3=XXXW, 4=YWYX, 5=WYXY, 6=YYWX, 7=YWXX, 8=WYXX, 9=XYYW, 10=XYWX, 11=YYXW, 12=WYYX, 13=XYXW, 14=WYYY, 15=WXYW, 16=WYXW, 17=WXXW, 18=WYYW, 19=XYYX, 20=YXYX, 21=YXXY and 22=XYXY.

Once the 4mers are assigned to positions according to the above pattern, a particular set of oligonucleotides can be created by appropriate assignment of bases, A, T/U, G, C to W, X, Y. These assignments are made according to one of the following two sets of rules:
(i) Each of W, X and Y is a base in which:
   (a) W=one of A, T/U, G, and C,
      X=one of A, T/U, G, and C,
      Y=one of A, T/U, G, and C,
      and each of W, X and Y is selected so as to be different from all of the others of W, X and Y,
   (b) an unselected said base of (i)(a) can be substituted any number of times for any one of W, X and Y.
      or
(ii) Each of W, X and Y is a base in which:
   (a) W=G or C,
      X=A or T/U,
      Y=A or T/U,
      and X≠Y, and
   (b) a base not selected in (ii)(a) can be inserted into each sequence at one or more locations, the location of each insertion being the same in each sequence as that of every o sequence of the set;

In the case of the specific oligonucleotides given in Table I, W=G, X=A and Y=T.

In any case, given a set of oligonucleotides generated according to one of these sets of rules, it is possible to modify the members of a given set in relatively minor ways and thereby obtain a different set of sequences while more or less maintaining the cross-hybridization properties of the set subject to such modification. In particular, it is possible to insert up to 3 of A, T/U, G and C at any location of any sequence of the set of sequences. Alternatively, or additionally, up to 3 bases can be deleted from any sequence of the set of sequences.

A person skilled in the art would understand that given a set of oligonucleotides having a set of properties making it suitable for use as a family of tags (or tag complements) one can obtain another family with the same property by reversing the order of all of the members of the set. In other words, all the members can be taken to be read 51 to 31 or to be read 3′ to 5′.

A family of complements of the present invention is based on a given set of oligonucleotides defined as described above. Each complement of the family is based on a different oligonucleotide of the set and each complement contains at least 10 consecutive (i.e., contiguous) bases of the oligonucleotide on which it is based. For a given family of complements where one is seeking to reduce or minimize inter-sequence similarity that would result in cross-hybridization, each and every pair of complements meets particular homology requirements. Particularly, subject to limited exceptions, described below, any two complements within a set of complements are generally required to have a defined amount of dissimilarity.

In order to notionally understand these requirements for dissimilarity as they exist for a given pair of complements of a family, a phantom sequence is generated from the pair of complements. A “phantom” sequence is a single sequence that is generated from a pair of complements by selection, from each complement of the pair, of a string of bases wherein the bases of the string occur in the same order in both complements. An object of creating such a phantom sequence is to create a convenient and objective means of comparing the sequence identity of the two parent sequences from which the phantom sequence is created.

A phantom sequence may thus be generated from exemplary Sequence 1 and Sequence 2 as follows:

| | |
|---|---|
| Sequence 1: | ATGTTTAGTGAAAAGTTAGTATTG |
| | * • |
| Sequence 2: | ATGTTAGTGAATAGTATAGTATTG |
| | • ♦ |
| Phantom Sequence: | ATGTTAGTGAAAGTTAGTATTG |

The phantom sequence generated from these two sequences is thus 22 bases in length. That is, one can see that there are 22 identical bases with identical sequence (the same order) in Sequence Nos. 1 and 2. There is a total of three insertions/deletions and mismatches present in the phantom sequence when compared with the sequences from which it was generated:
ATGT-TAGTGAA-AGT-TAGTATTG
   The dashed lines in this latter representation of the phantom sequence indicate the locations of the insertions/deletions and mismatches in the phantom sequence relative to the parent sequences from which it was derived. Thus, the “T” marked with an asterisk in Sequence 1, the “A” marked with a diamond in Sequence 2 and the “A-T” mismatch of Sequences 1 and 2 marked with two dots were deleted in generating the phantom sequence.

A person skilled in the art will appreciate that the term “insertion/deletion” is intended to cover the situations indicated by the asterisk and diamond. Whether the change is considered, strictly speaking, an insertion or deletion is merely one of vantage point. That is, one can see that the fourth base of Sequence 1 can be deleted therefrom to obtain the phantom sequence, or a “T” can be inserted after the third base of the phantom sequence to obtain Sequence 1.

One can thus see that if it were possible to create a phantom sequence by elimination of a single insertion/deletion from one of the parent sequences, that the two parent sequences would have identical homology over the length of the phantom sequence except for the presence of a single base in one of the two sequences being compared. Likewise, one can see that if it were possible to create a phantom sequence through deletion of a mismatched pair of bases, one base in each parent, that the two parent sequences would have identical homology over the length of the phantom sequence except for the presence of a single base in each of the sequences being compared. For this reason, the effect of an insertion/deletion is considered equivalent to the effect of a mismatched pair of bases when comparing the homology of two sequences.

Once a phantom sequence is generated, the compatibility of the pair of complements from which it was generated within a family of complements can be systematically evaluated:

According to one embodiment of the invention, a pair of complements is compatible for inclusion within a family of complements if any phantom sequence generated from the pair of complements has the following properties:
Any consecutive sequence of bases in the phantom sequence which is identical to a consecutive sequence of bases in each of the first and second complements from which it is generated is no more ((¾×L)−1) bases in length;
The phantom sequence, if greater than or equal to (⅚×L) in length, contains at least 3 insertions/deletions or mismatches when compared to t first and second complements from which it is generated; and
The phantom sequence is not greater than or equal to ( 11/12×L) in length.

Here, L₁ is the length of the first complement, L₂ is the length of the second complement, and L=L₁, or if L₁≠L₂, L is the greater of L₁ and L₂.

In particular preferred embodiments of the invention, all pairs of complements of a given set have the properties set out above. Under particular circumstances, it may be advantageous to have a limited number of complements that do not meet all of these requirements when compared to every other complement in a family.

In one case, for any first complement there are at most two second complements in the family which do not meet all of the three listed requirements. For two such complements, there would thus be a greater chance of cross-hybridization between their tag counterparts and the first complement. In another case, for any first complement there is at most one second complement which does not meet all of three listed requirements.

It is also possible, given this invention, to design a family of complements where a specific number or specific portion of the complements do not meet the three listed requirements. For example, a set could be designed where only one pair of complements within the set do not meet the requirements when compared to each other. There could be two pairs, three pairs, and any number of pairs up to and including all possible pairs. Alternatively, it may be advantageous to have a given proportion of pairs of complements that do not meet the requirements, say 10% of pairs, when compared with other sequences that do not meet one or more of the three requirements listed. This number could instead by 5%, 15%, 20%, 25%, 30%, 35%, or 40%.

The foregoing comparisons would generally be largely carried out using appropriate computer software. Although notionally described in terms of a phantom sequence for the sake of clarity and understanding, it will be understood that a competent computer programmer can carry out pairwise comparisons of complements in any number of ways using logical steps that obtain equivalent results.

The symbols A, G, T/U; C take on their usual meaning in the art here. In the case of T and U, a person skilled in the art would understand that these are equivalent to each other with respect to the inter-strand hydrogen-bond (Watson-Crick) binding properties at work in the context of this invention. The two bases are thus interchangeable and hence the designation of T/U.

Analogues of the naturally occurring bases can be inserted in their respective places where desired. Analogues can be defined as any non-natural base, such as peptide nucleic acids and the like.

Other aspects of the invention are described below, particularly numbered paragraphs at the end of this specification.

In another broad embodiment A family of 1168 sequences was obtained using a computer algorithm to have desirable hybridization properties for use in nucleic acid detection assays. The sequence set of 1168 oligonucleotides was partially characterized in hybridization assays, demonstrating the ability of family members to correctly hybridize to their complementary sequences with minimal cross hybridization. These are the sequences having SEQ ID NOs:1 to 1168 of Table II.

Variant families of sequences (seen as tags or tag complements) of a family of sequences taken from Table II are also part of the invention. For the purposes of discussion, a family or set of oligonucleotides will-often be described as a family of tag complements, but it will be understood that such a set could just easily be a family of tags.

A family of complements is obtained from a set of oligonucleotides based on a family of oligonucleotides such as those of Table II. To simplify discussion, providing a family of complements based on the oligonucleotides of Table II will be described.

Firstly, the groups of sequences based on the oligonucleotides of Table II can be represented as shown in Table IIA.

TABLE IIA
Numeric sequences corresponding to nucleotide patterns of a set of oligonucleotides

| | | | | | | | | | | | | | | | | | | | | | | | | Sequence |
|---|---|---|---|---|---|---|---|---|---|---|---|---|---|---|---|---|---|---|---|---|---|---|---|---|
| Numeric Pattern | | | | | | | | | | | | | | | | | | | | | | | | Identifier |
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 | 2 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 | 3 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 | 4 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 | 5 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 | 6 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 | 7 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 | 8 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 | 9 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 | 10 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 | 11 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 | 12 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 | 13 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 | 14 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 | 15 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 | 16 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 | 17 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 | 18 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 | 19 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 | 20 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 | 21 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 | 22 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 | 23 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 | 24 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 | 25 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 | 26 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 | 27 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 | 28 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 | 29 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 | 30 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 | 31 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 | 32 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 | 33 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 | 34 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 | 35 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 | 36 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 | 37 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 | 38 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 | 39 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 | 40 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 | 41 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 | 42 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 | 43 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 | 44 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 | 45 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 | 46 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 | 47 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 | 48 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 | 49 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 | 50 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 | 51 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 | 52 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 | 53 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 | 54 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 | 55 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 | 56 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 | 57 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 | 58 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 | 59 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 | 60 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 | 61 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 | 62 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 | 63 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 | 64 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 | 65 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 | 66 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 | 67 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 | 68 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 | 69 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 | 70 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 | 71 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 | 72 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 | 73 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 | 74 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 | 75 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 | 76 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 | 77 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 | 78 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 | 79 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 | 80 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 | 81 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 | 82 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 | 83 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 | 84 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 | 85 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 | 86 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 | 87 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 | 88 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 | 89 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 | 90 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 | 91 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 | 92 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 | 93 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 | 94 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 | 95 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 | 96 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 | 97 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 | 98 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 | 99 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 | 100 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 | 101 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 | 102 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 | 103 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 | 104 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 | 105 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 | 106 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 | 107 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 | 108 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 | 109 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 | 110 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 | 111 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 | 112 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 | 113 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 | 114 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 | 115 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 | 116 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 | 117 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 | 118 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 | 119 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 | 120 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 | 121 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 | 122 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 | 123 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 | 124 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 | 125 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 | 126 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 | 127 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 | 128 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 | 129 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 | 130 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 | 131 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 | 132 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 | 133 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 | 134 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 | 135 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 | 136 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 | 137 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 | 138 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 | 139 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 | 140 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 | 141 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 | 142 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 | 143 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 | 144 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 | 145 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 | 146 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 | 147 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 | 148 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 | 149 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 | 150 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 | 151 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 | 152 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 | 153 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 | 154 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 | 155 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 | 156 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 | 157 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 | 158 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 | 159 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 | 160 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 | 161 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 | 162 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 | 163 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 | 164 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 | 165 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 | 166 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 | 167 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 | 168 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 | 169 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 | 170 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 | 171 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 | 172 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 | 173 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 | 174 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 | 175 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 | 176 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 | 177 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 | 178 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 | 179 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 | 180 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 | 181 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 | 182 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 | 183 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 | 184 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 | 185 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 | 186 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 | 187 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 | 188 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 | 189 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 | 190 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 | 191 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 | 192 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 | 193 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 | 194 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 | 195 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 | 196 |
| 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 | 197 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 | 198 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 | 199 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 | 200 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 | 201 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 | 202 |
| 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 | 203 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 | 204 |
| 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 | 205 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 | 206 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 | 207 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 | 208 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 | 209 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 | 210 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 | 211 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 | 212 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 | 213 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 | 214 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 | 215 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 | 216 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 | 217 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 | 218 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 | 219 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 | 220 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 | 221 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 | 222 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 | 223 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 | 224 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 | 225 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 | 226 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 | 227 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 | 228 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 | 229 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 | 230 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 | 231 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 | 232 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 | 233 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 | 234 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 | 235 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 | 236 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 | 237 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 | 238 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 | 239 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 | 240 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 | 241 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 | 242 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 | 243 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 | 244 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 | 245 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 | 246 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 | 247 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 | 248 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 | 249 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 | 250 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 | 251 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 | 252 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 | 253 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 | 254 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 | 255 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 | 256 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 | 257 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 | 258 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 | 259 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 | 260 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 | 261 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 | 262 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 | 263 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 | 264 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 | 265 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 | 266 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 | 267 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 | 268 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 | 269 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 | 270 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 | 271 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 | 272 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 | 273 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 | 274 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 | 275 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 | 276 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 | 277 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 | 278 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 | 279 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 | 280 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 | 281 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 | 282 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 | 283 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 | 284 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 | 285 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 | 286 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 | 287 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 | 288 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 | 289 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 | 290 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 | 291 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 | 292 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 | 293 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 | 294 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 | 295 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 | 296 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 | 297 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 | 298 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 | 299 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 | 300 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 | 301 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 | 302 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 | 303 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 | 304 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 | 305 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 | 306 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 | 307 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 | 308 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 | 309 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 | 310 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 | 311 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 | 312 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 | 313 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 | 314 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 | 315 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 | 316 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 | 317 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 | 318 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 | 319 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 | 320 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 | 321 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 | 322 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 | 323 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 | 324 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 | 325 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 | 326 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 | 327 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 | 328 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 | 329 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 | 330 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 | 331 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 | 332 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 | 333 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 | 334 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 | 335 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 | 336 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 | 337 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 | 338 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 | 339 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 | 340 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 | 341 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 | 342 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 | 343 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 | 344 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 | 345 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 | 346 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 | 347 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 | 348 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 | 349 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 | 350 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 | 351 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 | 352 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 | 353 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 | 354 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 | 355 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 | 356 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 | 357 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 | 358 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 | 359 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 | 360 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 | 361 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 | 362 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 | 363 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 | 364 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 | 365 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 | 366 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 | 367 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 | 368 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 | 369 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 | 370 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 | 371 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 | 372 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 | 373 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 | 374 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 | 375 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 | 376 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 | 377 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 | 378 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 | 379 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 | 380 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 | 381 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 | 382 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 | 383 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 | 384 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 | 385 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 | 386 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 | 387 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 | 388 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 | 389 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 | 390 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 | 391 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 | 392 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 | 393 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 | 394 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 | 395 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 | 396 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 | 397 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 | 398 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 | 399 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 | 400 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 | 401 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 | 402 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 | 403 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 | 404 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 | 405 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 | 406 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 | 407 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 | 408 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 | 409 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 | 410 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 | 411 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 | 412 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 | 413 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 | 414 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 | 415 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 | 416 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 | 417 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 | 418 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 | 419 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 | 420 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 | 421 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 | 422 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 | 423 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 | 424 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 | 425 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 | 426 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 | 427 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 | 428 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 | 429 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 | 430 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 | 431 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 | 432 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 | 433 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 | 434 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 | 435 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 | 436 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 | 437 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 | 438 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 | 439 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 | 440 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 | 441 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 | 442 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 | 443 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 | 444 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 | 445 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 | 446 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 | 447 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 | 448 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 | 449 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 | 450 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 | 451 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 | 452 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 | 453 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 | 454 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 | 455 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 | 456 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 | 457 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 | 458 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 | 459 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 | 460 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 | 461 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 | 462 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 | 463 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 | 464 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 | 465 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 | 466 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 | 467 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 | 468 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 | 469 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 | 470 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 | 471 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 | 472 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 | 473 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 | 474 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 | 475 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 | 476 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 | 477 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 | 478 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 | 479 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 | 480 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 | 481 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 | 482 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 | 483 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 | 484 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 | 485 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 | 486 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 | 487 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 | 488 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 | 489 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 | 490 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 | 491 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 | 492 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 | 493 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 | 494 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 | 495 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 | 496 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 | 497 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 | 498 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 | 499 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 | 500 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 | 501 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 | 502 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 | 503 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 | 504 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 | 505 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 | 506 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 | 507 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 | 508 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 | 509 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 | 510 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 | 511 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 | 512 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 | 513 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 | 514 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 | 515 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 | 516 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 | 517 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 | 518 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 | 519 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 | 520 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 | 521 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 | 522 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 | 523 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 | 524 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 | 525 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 | 526 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 | 527 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 | 528 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 | 529 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 | 530 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 | 531 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 | 532 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 | 533 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 | 534 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 | 535 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 | 536 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 | 537 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 | 538 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 | 539 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 | 540 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 | 541 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 | 542 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 | 543 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 | 544 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 | 545 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 | 546 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 | 547 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 | 548 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 | 549 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 | 550 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 | 551 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 | 552 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 | 553 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 | 554 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 | 555 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 | 556 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 | 557 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 | 558 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 | 559 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 | 560 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 | 561 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 | 562 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 | 563 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 | 564 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 | 565 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 | 566 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 | 567 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 | 568 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 | 569 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 | 570 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 | 571 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 | 572 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 | 573 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 | 574 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 | 575 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 | 576 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 | 577 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 | 578 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 | 579 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 | 580 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 | 581 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 | 582 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 | 583 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 | 584 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 | 585 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 | 586 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 | 587 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 | 588 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 | 589 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 | 590 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 | 591 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 | 592 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 | 593 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 | 594 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 | 595 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 | 596 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 | 597 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 | 598 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 | 599 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 | 600 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 | 601 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 | 602 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 | 603 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 | 604 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 | 605 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 | 606 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 | 607 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 | 608 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 | 609 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 | 610 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 | 611 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 | 612 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 | 613 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 | 614 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 | 615 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 | 616 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 | 617 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 | 618 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 | 619 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 | 620 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 | 621 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 | 622 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 | 623 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 | 624 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 | 625 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 | 626 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 | 627 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 | 628 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 | 629 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 | 630 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 | 631 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 | 632 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 | 633 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 | 634 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 | 635 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 | 636 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 | 637 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 | 638 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 | 639 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 | 640 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 | 641 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 | 642 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 | 643 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 | 644 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 | 645 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 | 646 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 | 647 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 | 648 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 | 649 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 | 650 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 | 651 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 | 652 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 | 653 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 | 654 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 | 655 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 | 656 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 | 657 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 | 658 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 | 659 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 | 660 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 | 661 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 | 662 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 | 663 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 | 664 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 | 665 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 | 666 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 | 667 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 | 668 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 | 669 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 | 670 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 | 671 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 | 672 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 | 673 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 | 674 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 | 675 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 | 676 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 | 677 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 | 678 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 | 679 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 | 680 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 | 681 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 | 682 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 | 683 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 | 684 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 | 685 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 | 686 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 | 687 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 | 688 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 | 689 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 | 690 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 | 691 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 | 692 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 | 693 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 | 694 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 | 695 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 | 696 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 | 697 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 | 698 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 | 699 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 | 700 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 | 701 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 | 702 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 | 703 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 | 704 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 | 705 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 | 706 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 | 707 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 | 708 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 | 709 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 | 710 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 | 711 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 | 712 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 | 713 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 | 714 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 | 715 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 | 716 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 | 717 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 | 718 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 | 719 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 | 720 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 | 721 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 | 722 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 | 723 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 | 724 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 | 725 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 | 726 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 | 727 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 | 728 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 | 729 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 | 730 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 | 731 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 | 732 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 | 733 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 | 734 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 | 735 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 | 736 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 | 737 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 | 738 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 | 739 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 | 740 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 | 741 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 | 742 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 | 743 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 | 744 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 | 745 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 | 746 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 | 747 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 | 748 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 | 749 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 | 750 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 | 751 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 | 752 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 | 753 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 | 754 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 | 755 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 | 756 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 | 757 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 | 758 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 | 759 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 | 760 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 | 761 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 | 762 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 | 763 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 | 764 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 | 765 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 | 766 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 | 767 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 | 768 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 | 769 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 | 770 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 | 771 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 | 772 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 | 773 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 | 774 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 | 775 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 | 776 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 | 777 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 | 778 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 | 779 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 | 780 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 | 781 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 | 782 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 | 783 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 | 784 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 | 785 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 | 786 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 | 787 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 | 788 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 | 789 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 | 790 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 | 791 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 | 792 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 | 793 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 | 794 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 | 795 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 | 796 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 | 797 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 | 798 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 | 799 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 | 800 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 | 801 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 | 802 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 | 803 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 | 804 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 | 805 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 | 806 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 | 807 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 | 808 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 | 809 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 | 810 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 | 811 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 | 812 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 | 813 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 | 814 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 | 815 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 | 816 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 | 817 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 | 818 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 | 819 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 | 820 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 | 821 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 | 822 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 | 823 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 | 824 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 | 825 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 | 826 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 | 827 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 | 828 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 | 829 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 | 830 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 | 831 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 | 832 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 | 833 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 | 834 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 | 835 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 | 836 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 | 837 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 | 838 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 | 839 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 | 840 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 | 841 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 | 842 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 | 843 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 | 844 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 | 845 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 | 846 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 | 847 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 | 848 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 | 849 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 | 850 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 | 851 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 | 852 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 | 853 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 | 854 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 | 855 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 | 856 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 | 857 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 | 858 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 | 859 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 | 860 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 | 861 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 | 862 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 | 863 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 | 864 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 | 865 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 | 866 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 | 867 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 | 868 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 | 869 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 | 870 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 | 871 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 | 872 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 | 873 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 | 874 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 | 875 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 | 876 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 | 877 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 | 878 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 | 879 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 | 880 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 | 881 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 | 882 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 | 883 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 | 884 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 | 885 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 | 886 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 | 887 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 | 888 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 | 889 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 | 890 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 | 891 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 | 892 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 | 893 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 | 894 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 | 895 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 | 896 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 | 897 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 | 898 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 | 899 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 | 900 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 | 901 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 | 902 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 | 903 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 | 904 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 | 905 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 | 906 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 | 907 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 | 908 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 | 909 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 | 910 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 | 911 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 | 912 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 | 913 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 | 914 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 | 915 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 | 916 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 | 917 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 | 918 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 | 919 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 | 920 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 | 921 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 | 922 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 | 923 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 | 924 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 | 925 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 | 926 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 | 927 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 | 928 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 | 929 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 | 930 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 | 931 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 | 932 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 | 933 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 | 934 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 | 935 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 | 936 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 | 937 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 | 938 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 | 939 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 | 940 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 | 941 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 | 942 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 | 943 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 | 944 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 | 945 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 | 946 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 | 947 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 | 948 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 | 949 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 | 950 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 | 951 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 | 952 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 | 953 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 | 954 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 | 955 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 | 956 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 | 957 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 | 958 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 | 959 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 | 960 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 | 961 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 | 962 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 | 963 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 | 964 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 | 965 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 | 966 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 | 967 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 | 968 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 | 969 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 | 970 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 | 971 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 | 972 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 | 973 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 | 974 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 | 975 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 | 976 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 | 977 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 | 978 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 | 979 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 | 980 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 | 981 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 | 982 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 | 983 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 | 984 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 | 985 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 | 986 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 | 987 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 | 988 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 | 989 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 | 990 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 | 991 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 | 992 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 | 993 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 | 994 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 | 995 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 | 996 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 | 997 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 | 998 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 | 999 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 | 1000 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 | 1001 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 | 1002 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 | 1003 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 | 1004 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 | 1005 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 | 1006 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 | 1007 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 | 1008 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 | 1009 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 | 1010 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 | 1011 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 | 1012 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 | 1013 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 | 1014 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 | 1015 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 | 1016 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 | 1017 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 | 1018 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 | 1019 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 | 1020 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 | 1021 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 | 1022 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 | 1023 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 | 1024 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 | 1025 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 | 1026 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 | 1027 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 | 1028 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 | 1029 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 | 1030 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 | 1031 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 | 1032 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 | 1033 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 | 1034 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 | 1035 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 | 1036 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 | 1037 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 | 1038 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 | 1039 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 | 1040 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 | 1041 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 | 1042 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 | 1043 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 | 1044 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 | 1045 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 | 1046 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 | 1047 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 | 1048 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 | 1049 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 | 1050 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 | 1051 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 | 1052 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 | 1053 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 | 1054 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 | 1055 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 | 1056 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 | 1057 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 | 1058 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 | 1059 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 | 1060 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 | 1061 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 | 1062 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 | 1063 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 | 1064 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 | 1065 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 | 1066 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 | 1067 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 | 1068 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 | 1069 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 | 1070 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 | 1071 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 | 1072 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 | 1073 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 | 1074 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 | 1075 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 | 1076 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 | 1077 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 | 1078 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 | 1079 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 | 1080 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 | 1081 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 | 1082 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 | 1083 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 | 1084 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 | 1085 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 | 1086 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 | 1087 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 | 1088 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 | 1089 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 | 1090 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 | 1091 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 | 1092 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 | 1093 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 | 1094 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 | 1095 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 | 1096 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 | 1097 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 | 1098 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 | 1099 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 | 1100 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 | 1101 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 | 1102 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 | 1103 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 | 1104 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 | 1105 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 | 1106 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 | 1107 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 | 1108 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 | 1109 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 | 1110 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 | 1111 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 | 1112 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 | 1113 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 | 1114 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 | 1115 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | T | 1 | 1116 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 | 1117 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 | 1118 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 | 1119 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 | 1120 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 | 1121 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 | 1122 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 | 1123 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 | 1124 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 | 1125 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 | 1126 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 | 1127 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 | 1128 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 | 1129 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 | 1130 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 | 1131 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 | 1132 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 | 1133 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 | 1134 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 | 1135 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 | 1136 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 | 1137 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 | 1138 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 | 1139 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 | 1140 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 | 1141 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 | 1142 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 | 1143 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 | 1144 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 | 1145 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 | 1146 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 | 1147 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 | 1148 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 | 1149 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 | 1150 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 | 1151 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 | 1152 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 | 1153 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 | 1154 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 | 1155 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 | 1156 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 | 1157 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 | 1158 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 | 1159 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 | 1160 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 | 1161 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 | 1162 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 | 1163 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 | 1164 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 | 1165 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 | 1166 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 | 1167 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 | 1168 |

In Table IIA, each of the numerals 1 to 3 (numeric identifiers) represents a nucleotide base and the pattern of numerals 1 to 3 of the sequences the above list corresponds to the pattern of nucleotide bases present in the oligonucleotides of Table II, which oligonucleotides have been found to be non-cross-hybridizing, as described further in the detailed examples. Each nucleotide base is selected from the group of nucleotide bases consisting of A, C, G, and T/U. A particularly preferred embodiment of the invention, in which a specific base is assigned to each numeric identifier is shown in Table II, below.

In one broad aspect, the invention is a composition comprising molecules for use as tags or tag complements wherein each molecule comprises an oligonucleotide selected from a set of oligonucleotides based on a group of sequences as specified by numeric identifiers set out in Table IIA. In the sequences, each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for
any alignment of maximum score; and
M5 is the maximum sum of the lengths of all the blocks of matches having a length of at least 3, for any alignment of maximum score; wherein:
   the score of an alignment is determined according to the equation (A×m)−(B×mm)−(C×(og+eg))−(D−eg)), wherein:
      for each of (i) to (iv):
         (i) m=6, mm=6, og=0 and eg=6,
         (ii) m=6, mm=6, og=5 and eg=1,
         (iii) m=6, mm=2, og=5 and eg=1, and
         (iv) m=6, mm=6., og=6 and eg=0,
      A is the total number of matched pairs of bases in the alignment;
      B is the total number of internal mismatched pairs in the alignment;
      C is the total number of internal gaps in the alignment; and.
      D is the total number of internal indels in the alignment minus
      the total number of internal gaps in the alignment; and
   wherein the maximum score is determined separately for each of (i), (ii), (iii) and (iv).

An explanation of the meaning of the parameters set out above is given in the section describing detailed embodiments.

In another broad aspect the invention is a composition containing molecules for use as tags or tag complements wherein each molecule comprises an oligonucleotide selected from a set of oligonucleotides based on a group of sequences as set out in Table IIA wherein each of 1 to 3 is a nucleotide base selected to be different from the others of 1 to 3 with the proviso that up to three nucleotide bases of each sequence can be substituted with any nucleotide base provided that:
for any pair of sequences of the set:
   M1≦19, M2≦17, M3≦21, M4≦18, and M5≦20, where:
      M1 is the maximum number of matches for any alignment in which there are no internal indels;
      M2 is the maximum length of a block of matches for any alignment;
      M3 is the maximum number of matches for any alignment having a maximum score;
      M4 is the maximum sum of the lengths of the longest two blocks of matches for any alignment of maximum score; and
      M5 is the maximum sum of the lengths of all the blocks of matches having a length of at least 3, for any alignment of maximum score; wherein
         the score of an alignment is determined according to the equation (A×m)−(B×mm)−(C×(og+eg))−(D×eg)), wherein:
            for each of (i) to (iv)
            (i) m=6, mm=6, og=0 and eg=6,
            (ii) m=6, mm=6, og=5 and eg=1,
            (iii) m=6, mm=2, og=S and eg=1, and
            (iv) m=6, mm=6, og=6 and eg=0,
            A is the total number of matched pairs of bases in the alignment;
            B is the total number of internal mismatched pairs in the alignment;
            C is the total number of internal gaps in the alignment; and
            D is the total number of internal indels in the alignment minus the total number of internal gaps in the alignment; and
         wherein the maximum score is determined separately for each of (i), (ii), (iii) and (iv).

In another broad aspect, the invention is a composition comprising molecules for use as tags or tag complements wherein each molecule comprises an oligonucleotide selected from a set of oligonucleotides based on a group of sequences set out in Table IIA wherein each of 1 to 3 is a nucleotide base selected to be different from the others of 1 to 3 with the proviso that up to three nucleotide bases of each sequence can be substituted with any nucleotide base provided that:
for any pair of sequences of the set:
   M1≦19, M2≦17, M3≦21, M4≦18, and M5≦20, where:
      M1 is the maximum number of matches for any alignment in which there are n internal indels;
      M2 is the maximum length of a block of matches for any alignment;
      M3 is the maximum number of matches for any alignment having a maximum score;
      M4 is the maximum sum of the lengths of the longest two blocks of matches for any alignment of maximum score; and
      M5 is the maximum sum of the lengths of all the blocks of matches having a length of at least 3, for any alignment of maximum score, wherein:
         the score of an alignment is determined according to the equation 3A−B−3C−D, wherein:
            A is the total number of matched pairs of bases in the alignment;
            B is the total number of internal mismatched pairs in the alignment;
            C is the total number of internal gaps in the alignment; and
            D is the total number of internal indels in the alignment minus
            the total number of internal gaps in the alignment; and

In preferred aspects, the invention provides a composition in which, for the group of 24mer sequences in which 1=A, 2=T and 3=G, under a defined set of conditions in which the maximum degree of hybridization between a sequence and any complement of a different sequence of the group of. 24mer sequences does not exceed 30% of the degree of hybridization between said sequence and its complement, for all said oligonucleotides of the composition, the maximum degree of hybridization between an oligonucleotide and a complement of any other oligonucleotide of the composition does not exceed 50% of the degree of hybridization of the oligonucleotide and its complement.

More preferably, the maximum degree of hybridization between a sequence and any complement of a different sequence does not exceed 30% of the degree of hybridization between said sequence and its complement, the degree of hybridization between each sequence and its complement varies by a factor of between 1 and up to 10, more preferably between 1 and up to 9, more preferably between 1 and up to 8, more preferably between 1 and up to 7, more preferably between 1 and up to 6, and more preferably between 1 and up to 5.

It is also preferred that the maximum degree of hybridization between a sequence and any complement of a different sequence does not exceed 25%, more preferably does not exceed 20.%, more preferably does not exceed 15%, more preferably does not exceed 10%, more preferably does not exceed 5%.

Even more preferably, the above-referenced defined set of conditions results in a level of hybridization that is the same as the level of hybridization obtained when hybridization conditions include 0.2 M NaCl, 0.1 M Tris, 0.08% Triton X-100, pH 8.0 at 37° C.

In the composition, the defined set of conditions can include the group of 24mer sequences being covalently linked to beads.

In a particular preferred aspect, for the group of 24mers the maximum degree of hybridization between a sequence and any complement of a different sequence does not exceed 15% of the degree of hybridization between said sequence and its complement and the degree of hybridization between each sequence and its complement varies by a factor of between 1 and up to 9, and for all oligonucleotides of the set, the maximum degree of hybridization between an oligonucleotide and a complement of any other oligonucleotide of the set does not exceed 20% of the degree of hybridization of the oligonucleotide and its complement.

It is possible that each 1 is one of A, T/U, G and C; each 2 is one of A, T/U, G and C; and each 3 is one of A, T/U, G and C; and each of 1, 2 and 3 is selected so as to be different from all of the others of 1, 2 and 3. More preferably, 1 is A or T/U, 2 is A or T/U and 3 is G or C. Even more preferably, 1 is A, 2 is T/U, and 3 is G.

In certain preferred composition, each of the oligonucleotides is from twenty-two to twenty-six bases in length, or from twenty-three to twenty-five, and preferably, each oligonucleotide is of the same length as every other said oligonucleotide.

In a particularly preferred embodiment, each oligonucleotide is twenty-four bases in length.

It is preferred that no oligonucleotide contains more than four contiguous bases that are identical to each other.

It is also preferred that the number of G's in each oligonucleotide does not exceed L/4 where L is the number of bases in said sequence.

For reasons described below, the number of G's in each said oligonucleotide is preferred not to vary from the average number of G's in all of the oligonucleotides by more than one. Even more preferably, the number of G's in each said oligonucleotide is the same as-every other said oligonucleotide. In the embodiment disclosed below in which oligonucleotides were tested, the sequence of each was twenty-four bases in length and each oligonucleotide contained 6 G's.

It is also preferred that, for each nucleotide, there is at most six bases other than G between every pair of neighboring pairs of G's.

Also, it is preferred that, at the 5′-end of each oligonucleotide at least one of the first, second, third, fourth, fifth, sixth and seventh bases of the sequence of the oligonculeotide is a G. Similarly, it is preferred, at the 3′-end of each oligonucleotide that at least one of the first, second, third, fourth, fifth, sixth and seventh bases of the sequence of the oligonucleotide is a G.

It is possible to have sequence compositions that include one hundred and sixty said molecules, or that include one hundred and seventy said molecules, or that include one hundred and eighty said molecules, or that include one hundred and ninety said molecules, or that include two hundred said molecules, or that include two hundred and twenty said molecules, or that include two hundred and forty said molecules, or that include two hundred and sixty said molecules, or that include two hundred and eighty said molecules, or that include three hundred said molecules, or that include four hundred said molecules, or that include five hundred said molecules, or that include six hundred said molecules, or that include seven hundred said molecules, or that include eight hundred said molecules, or that include nine hundred said molecules, or that include one thousand said molecules.

It is possible, in certain applications, for each molecule to be linked to a solid phase support so as to be distinguishable from a mixture containing other of the molecules by hybridization to its complement. Such a molecule can be linked to a defined location on a solid phase support such that the defined location for each molecule is different than the defined location for different others of the molecules.

In certain embodiments, each solid phase support is a microparticle and each said molecule is covalently linked to a different microparticle than each other different said molecule.

In another broad aspect, the invention is a composition comprising a set of 150 molecules for use as tags or tag complements wherein each molecule includes an oligonucleotide having a sequence of at least sixteen nucleotide bases wherein for any pair of sequences of the set:
M1≦19/24×L1, M2≦17/24×L1, M3≦21/24×L1, M4≦18/24×L1,
M5≦20/24×L1, where L1 is the length of the shortest sequence of the pair, where:
   M1 is the maximum number of matches for any alignment of the pair of sequences in which there are no internal indels;
   M2 is the maximum length of a block of matches for any alignment of the pair of sequences;
   M3 is the maximum number of matches for any alignment of the pair of sequences having a maximum score;
   M4 is the maximum sum of the lengths of the longest two blocks of matches for any alignment of the pair of sequences of maximum score; and
   M5 is the maximum sum of the lengths of all the blocks of matches having length of at least 3, for any alignment of the pair of sequences of maximum score, wherein:
      the score of an alignment is determined according to the equation (A×m)−(B×mm)−(C×(og+eg))−(D×eg)), wherein:
         for each of (i) to (iv):
         (i) m=6, mm=6, og=0 and eg=6,
         (ii) m=6, mm=6, og=5 and eg=1,
         (iii) m=6, mm=2, og=5 and eg=1, and
         (iv) m=6, mm=6, og=6 and eg=0,
         A is the total number of matched pairs of bases in the alignment;
         B is the total number of internal mismatched pairs in the alignment;
         C is the total number of internal gaps in the alignment; and
         D is the total number of internal indels in the alignment minus the total number of internal gaps in the alignment; and
      wherein the maximum score is determined separately for each of (i), (ii), (iii) and (iv).

In yet another broad aspect, the invention is a composition that includes a set of 150 molecules for use as tags or tag complements wherein each molecule has an oligonucleotide having a sequence of at least sixteen nucleotide bases wherein for any pair of sequences of the set:
M1≦19, M2≦17, M3≦21, M4≦18, and M5≦20, where:
   M1 is the maximum number of matches for any alignment of the pair of sequences in which there are no internal indels;
   M2 is the maximum length of a block of matches for any alignment of the pair of sequences;
   M3 is the maximum number of matches for any alignment of the pair of sequences having a maximum score;
   M4 is the maximum sum of the lengths of the longest two blocks of matches for any alignment of the pair of sequences of maximum score; and
   M5 is the maximum sum of the lengths of all the blocks of matches having a length of at least 3, for any alignment of the pair of sequences of maximum score, wherein:
      the score of a said alignment is determined according to the equation 3A−B−3C−D, wherein:
         A is the total number of matched pairs of bases in the alignment;
         B is the total number of internal mismatched pairs in the alignment;
         C is the total number of internal gaps in the alignment; and
         D is the total number of internal indels in the alignment minus the total number of internal gaps in the alignment.

In certain embodiments of the invention., each sequence of a composition has up to fifty bases. More preferably, however, each sequence is between sixteen and forty bases in length, or between sixteen and thirty-five bases in length, or between eighteen and thirty bases in length, or between twenty and twenty-eight bases in length, or between twenty-one and twenty-seven bases in length, or between twenty-two and twenty-six bases in length.

Often, each sequence is of the same length as every other said sequence. In particular embodiments disclosed-herein, each sequence is twenty-four bases in length.

Again, it can be preferred that no sequence contains more than four contiguous bases that are identical to each other, etc., as described above.

In certain preferred embodiments, the composition is such that, under a defined set of conditions, the maximum degree of hybridization between an oligonucleotide and any complement of a different oligonucleotide of the composition does not exceed about 30% of the degree of hybridization between said oligonucleotide and its complement, more preferably 20%, more preferably 15%, more preferably 10%, more preferably 6%.

Preferably, the set of conditions results in a level of hybridization that is the same as the level of hybridization obtained when hybridization conditions include 0.2 M NaCl, 0.1 M Tris, 0.08% Triton X-100, pH 8.0 at 37° C., and the oligonucleotides are covalently linked to microparticles. Of course it is possible that these specific conditions be used for determining the level of hybridization.

It is also preferred that under such a defined set of conditions, the degree of hybridization between each oligonucleotide and its complement varies by a factor of between 1 and up to 8, more preferably up to 7, more preferably up to 6, more preferably up to 5. In a particular disclosed embodiment, the observed variance in the degree of hybridization was a factor of only 5.3, i.e., the degree of hybridization between each oligonucleotide and its complement varied by a factor of between 1 and 5.6.

In certain preferred embodiments, under the defined set of conditions, the maximum degree of hybridization between a said oligonucleotide and any complement of a different oligonucleotide of the composition does not exceed about 15%, more preferably 10%, more preferably 6%.

In one preferred embodiment, the set of conditions results in a level of hybridization that is the same as the level of hybridization obtained when hybridization conditions include 0.2 M NaCl, 0.1 M Tris, 0.08% Triton X-100, pH 8.0 at 37° C., and the oligonucleotides are covalently linked to microparticles.

Also, under the defined set of conditions, it is preferred that the degree of hybridization between each oligonucleotide and its complement varies by a factor of between 1 and up to 8, more preferably up to 7, more preferably up to 6, more preferably up to 5.

Any composition of the invention can include one hundred and sixty of the oligonucleotide molecules, or one hundred and seventy of the oligonucleotide molecules, or one hundred and eighty of the oligonucleotide molecules, or one hundred and ninety of the oligonucleotide molecules, or two hundred of the oligonucleotide molecules, or two hundred and twenty of the oligonucleotide molecules, or two hundred and forty of the oligonucleotide molecules, or two hundred and sixty of the oligonucleotide molecules, or two hundred and eighty of the oligonucleotide molecules, or three hundred of the oligonucleotide molecules, or four hundred of the oligonucleotide molecules, or five hundred of the oligonucleotide molecules, or six hundred of the oligonucleotide molecules, or seven hundred of the oligonucleotide molecules, or eight hundred of the oligonucleotide molecules, or nine hundred of the oligonucleotide molecules, or one thousand or more of the oligonucleotide molecules.

A composition of the invention can be a family of tags, or it can be a family of tag complements.

An oligonucleotide molecule belonging to a family of molecules of the invention can have incorporated thereinto one more analogues of nucleotide bases, preference being given those that undergo normal Watson-Crick base pairing.

The invention includes kits for sorting and identifying polynucleotides. Such a kit can include one or more solid phase supports each having one or more spatially discrete regions, each such region having a uniform population of substantially identical tag complements covalently attached. The tag complements are made up of a set of oligonucleotides of the invention.

The one or more solid phase supports can be a planar substrate in which the one or more spatially discrete regions is a plurality of spatially addressable regions.

The tag complements can also be coupled to microparticles. Microparticles preferably each have a diameter in the range of from 5 to 40 μm.

Such a kit preferably includes microparticles that are spectrophotometrically unique, and therefore distinguisable from each other according to conventional laboratory techniques. Of course for such kits to work, each type of microparticle would generally have only one tag complement associated with it, and usually there would be a different oligonucleotide tag complement associated with (attached to) each type of microparticle.

The invention includes methods of using families of oligonucleotides of the invention.

One such method is of analyzing a biological sample containing a biological sequence for the presence of a mutation or polymorphism at a locus of the nucleic acid. The method includes:
(A) amplifying the nucleic acid molecule in the presence of a first primer having a 5′-sequence having the sequence of a tag complementary to the sequence of a tag complement belonging to a family of tag complements of the invention to form an amplified molecule with a 5′-end with a sequence complementary to the sequence of the tag;
(B) extending the amplified molecule in the presence of a polymerase and a second primer having 5′-end complementary the 3′-end of the amplified sequence, with the 3′-end of the second primer extending to immediately adjacent said locus, in the presence of a plurality of nucleoside triphosphate derivatives each of which is: (i) capable of incorporation during transciption by the polymerase onto the 3′-end of a growing nucleotide strand; (ii) causes termination of polymerization; and (iii) capable of differential detection, one from the other, wherein there is a said derivative complementary to each possible nucleotide present at said locus of the amplified sequence;
(C) specifically hybridizing the second primer to a tag complement having the tag complement sequence of (A); and
(D) detecting the nucleotide derivative incorporated into the second primer in (B) so as to identify the base located at the locus of the nucleic acid.

In another method of the invention, a biological sample containing a plurality of nucleic acid molecules is analyzed for the presence of a mutation or polymorphism at a locus of each nucleic acid molecule, for each nucleic acid molecule. This method includes steps of:
(A) amplifying the nucleic acid molecule in the presence of a first primer having a 5′-sequence having the sequence of a tag complementary to the sequence of a tag complement belonging to a family of tag complements of the invention to form an amplified molecule with a 5′-end with a sequence complementary to the sequence of the tag;
(B) extending the amplified molecule in the presence of a polymerase and a second primer having 5′-end complementary the 3′-end of the amplified sequence, the 3′-end of the second primer extending to immediately adjacent said locus, in the presence of a plurality of nucleoside triphosphate derivatives each of which is: (i) capable of incorporation during transciption by the polymerase onto the 3′-end of a growing nucleotide strand; (ii) causes termination of polymerization; and (iii) capable of differential detection, one from the other, wherein there is a said derivative complementary to each possible nucleotide present at said locus of the amplified molecule;
(C) specifically hybridizing the second primer to a tag complement having the tag complement sequence of (A); and
(D) detecting the nucleotide derivative incorporated into the second primer in (B) so as to identify the base located at the locus of the nucleic acid;
   wherein each tag of (A) is unique for each nucleic acid molecule and steps (A) a:
(B) are carried out with said nucleic molecules in the presence of each other.

Another method includes analyzing a biological sample that contains a plurality of double stranded complementary nucleic acid molecules for the presence of a mutation or polymorphism at a locus of each nucleic acid molecule, for each nucleic acid molecule. The method includes steps of:
(A) amplifying the double stranded molecule in the presence of a pair of first primers, each primer having an identical 5′-sequence having the sequence of a tag complementary to the sequence of a tag complement belonging to a family of tag complements of the invention to form amplified molecules with 5′-ends with a sequence complementary to the sequence of the tag;
(B) extending the amplified molecules in the presence of a polymerase and a p of second primers each second primer having a 5′-end complementary a 3′-end of the amplified sequence, the 3′-end of each said second primer extending to immediately adjacent said locus, in the presence of a plurality of nucleoside triphosphate derivatives each of which is: (i) capable of incorporation during transciption by the polymerase onto the 3′-end of a growing nucleotide strand; (ii) causes termination of polymerization; and (iii) capable of differential detection, one from the other;
(C) specifically hybridizing each of the second primers to a tag complement having the tag complement sequence of (A); and
(D) detecting the nucleotide derivative incorporated into the second primers in (B) so as to identify the base located at said locus;
   wherein the sequence of each tag of (A) is unique for each nucleic acid molecule and steps (A) and (B) are carried out with said nucleic molecules in the presence of each other.

In yet another aspect, the invention is a method of analyzing a biological sample containing a plurality of nucleic acid molecules for the presence of a mutation or polymorphism at a locus of each nucleic acid molecule, for each nucleic acid molecule, the method including steps of:
(a) hybridizing the molecule and a primer, the primer having a 5′-sequence having the sequence of a tag complementary to the sequence of a tag complement belonging to a family of tag complements of the invention and a 3′-end extending to immediately adjacent the locus;
(b) enzymatically extending the 3′-end of the primer in the presence of a plurality of nucleoside triphosphate derivatives each of which is: (i) capable of enzymatic incorporation onto the 3′-end of a growing nucleotide strand; (ii) causes termination of said extension; and (iii) capable of differential detection, one from the other, wherein there is a said derivative complementary to each possible nucleotide present at said locus;
(c) specifically hybridizing the extended primer formed in step (b) to a tag complement having the tag complement sequence of (a); and
(d) detecting the nucleotide derivative incorporated into the primer in step (b) so as to identify the base located at the locus of the nucleic a molecule;
   wherein each tag of (a) is unique for each nucleic acid molecule and steps (a) a (b) are carried out with said nucleic molecules in the presence of each other.

The derivative can be a dideoxy nucleoside triphosphate.

Each respective complement can be attached as a uniform population of substantially identical complements in spacially discrete regions on one or more solid phase support(s).

Each tag complement can include a label, each such label being different for respective complements, and step (d) can include detecting the presence of the different labels for respective hybridization complexes of bound tags and tag complements.

Another aspect of the invention includes a method of determining the presence of a target suspected of being contained in a mixture. The method includes the steps of:
(i) labelling the target with a first label;
(ii) providing a first detection moiety capable of specific binding to the target and including a first tag;
(iii) exposing a sample of the mixture to the detection moiety under conditions suitable to permit (or cause) said specific binding of the molecule and target;
(iv) providing a family of suitable tag complements of the invention wherein the family contains a first tag complement having a sequence complementary to that of the first tag;
(v) exposing the sample to the family of tag complements under-conditions suitable to permit (or cause) specific hybridization of the first tag and its tag complement;
(vi) determining whether a said first detection moiety hybridized to a first s tag complement is bound to a said labelled target in order to determine t presence or absence of said target in the mixture.

Preferably, the first tag complement is linked to a solid support at a specific location of the support and step (vi) includes detecting the presence of the first label at said specified location.

Also, the first tag complement can include a second label and step (vi) includes detecting the presence of the first and second labels in a hybridized complex of the moiety and the first tag complement.

Further, the target can be selected from the group consisting of organic molecules, antigens, proteins, polypeptides, antibodies and nucleic acids. The target can be an antigen and the first molecule can be an antibody specific for that antigen.

The antigen is usually a polypeptide or protein and the labelling step can include conjugation of fluorescent molecules, digoxigenin, biotinylation and the like.

The target can be a nucleic acid and the labelling step can include incorporation of fluorescent molecules, radiolabelled nucleotide, digoxigenin, biotinylation and the like.

Another aspect of the invention includes detecting the presence of a target nucleic acid molecule using the Invader Assay, which is described in detail in U.S. Pat. No. 5,985,557 issued Nov. 16, 1999, incorporated herein by reference. The sequences of the present invention are incorporated into the 3′ portion of one of the two oligonucleotide probes that will eventually be cleaved by a Cleavase enzyme and captured by its complement which may be attached on a solid phase support in a microarray format.

Another aspect of the invention includes a method of analyzing a biological sample comprising a plurality of target nucleic acid molecules for the presence of a mutation or polymorphism at a locus of each target nucleic acid molecule using the Invader Assay. Again, the sequences of the present invention are incorporated into the 3′ portion of one of the two probes that will eventually be cleaved by a Cleavase enzyme and detected by using the cleaved sequence's complement, which may be attached on a solid phase support such as in a microarray format.

Another aspect of the invention incorporates the use of a second target nucleic acid sequence, wherein the second target nucleic acid sequence comprises a synthetic nucleic acid. The synthetic nucleic acid may further comprise at least one hairpin loop. The construction and use of such nucleic acid sequences with hairpin loops has been described in detail in U.S. Pat. No. 5,770,365 issued Jun. 23, 1998 and International Publication WO 01/94625A2 published Dec. 13, 2001.

The present invention capitalizes on the exquisite specificity of the Invader Assay and the minimally cross-hybridizing sequences of the present invention such that simultaneous use of multiple hybridization probes in a single experiment is now possible. The methods and compositions of the present invention allow for accurate and homogenous genotyping of a plurality of distinct nucleic acid in a single experiment. The methods and compositions of the present invention are flexible enough to extend to novel loci with little optimization the features of both the Invader Assay and the sequences of the present invention lend the technology to automation.

### DETAILED DESCRIPTION OF THE INVENTION

Figures

Reference is made to the attached figures in which,

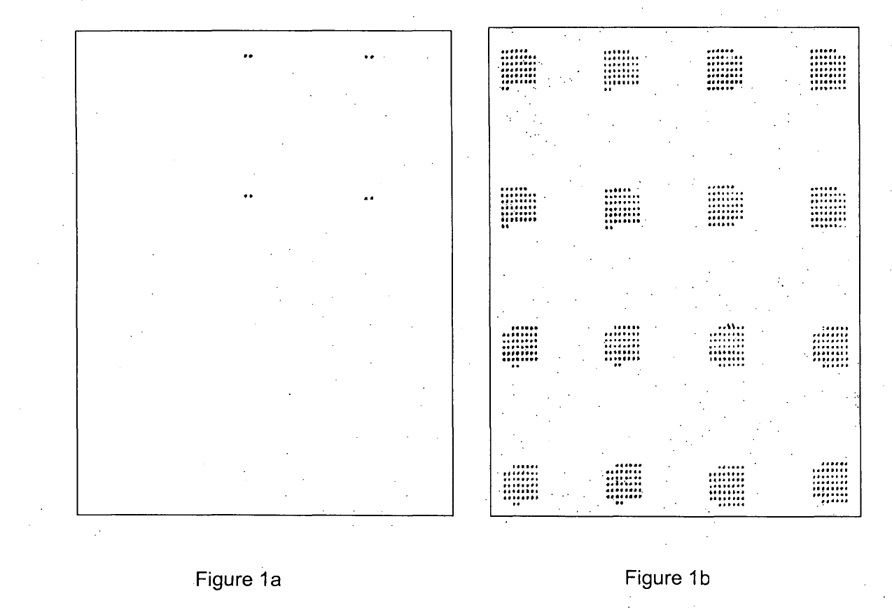

FIGS. 1A and 1B illustrate results obtained in the cross-hybridization experiments described in Example 1. FIG. 1A shows the hybridization pattern found when a microarray containing all 100 probes (SEQ ID NOs:1 to 100 of Table I) was hybridized with a 24mer oligonucleotide having the complementary sequence to SEQ ID NO:3 of Table I(target). FIG. 1B shows the pattern observed when a similar array was hybridized with a mix of all 100 targets, i.e., oligonucleotides having the sequences complementary to SEQ ID NOs:1 to 100 of Table 1.

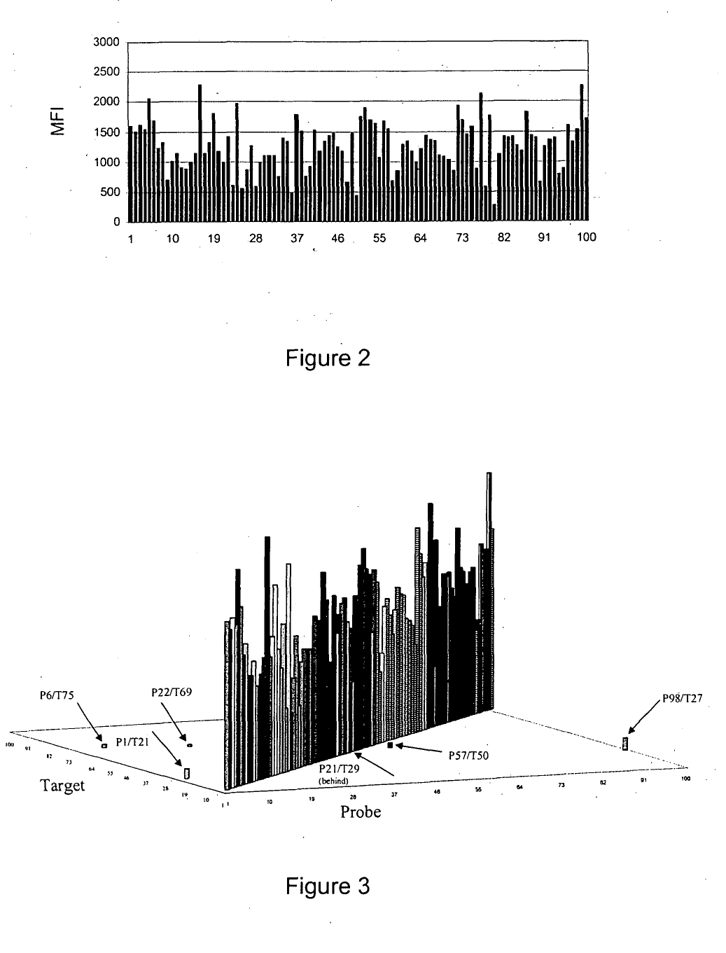

FIG. 2 shows the intensity of the signal (MFI) for each perfectly matched sequence (indicated in Table I) and its complement obtained as described in Example 3.

FIG. 3 is a three dimensional representation showing cross-hybridization observed for the sequences of FIG. 2 as described in Example 3. The results shown in FIG. 2 are reproduced along the diagonal of the drawing.

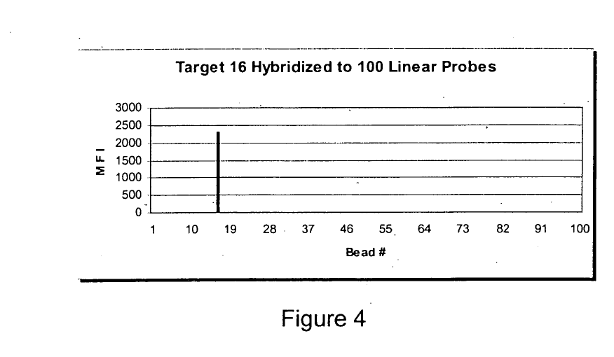

FIG. 4 is illustrative of results obtained for an individual target (SEQ ID NO:23 of Table I, target No. 16) when exposed to the 100 probes of Example 3. The MFI for each bead is plotted.

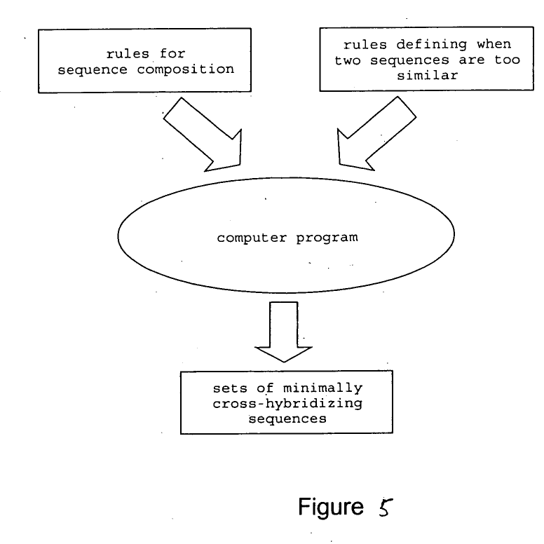

FIG. 5 illustrates generally the steps followed to obtain a family of sequences of the present invention;

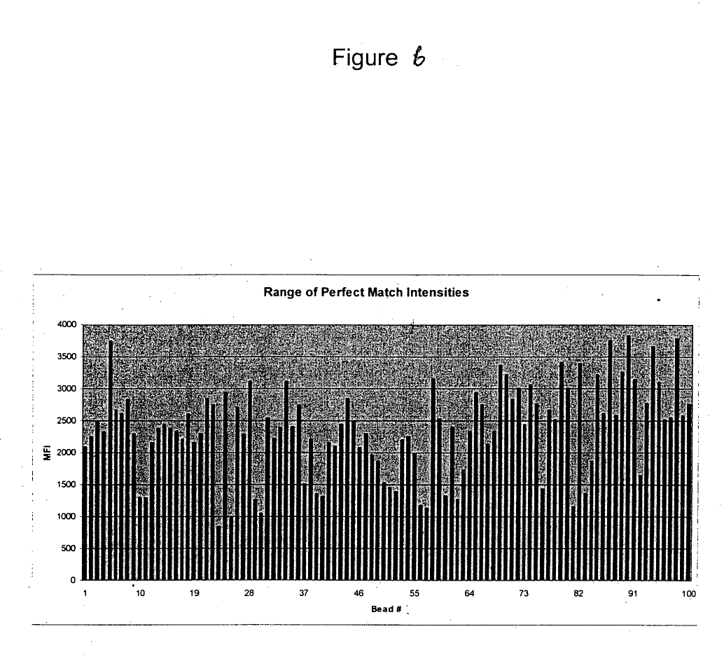

FIG. 6 shows the intensity of the signal (MFI) for each perfectly matched sequence (probe sequence indicated in Table II) and its complement (target at 50 fmol) obtained as described in Example 4;

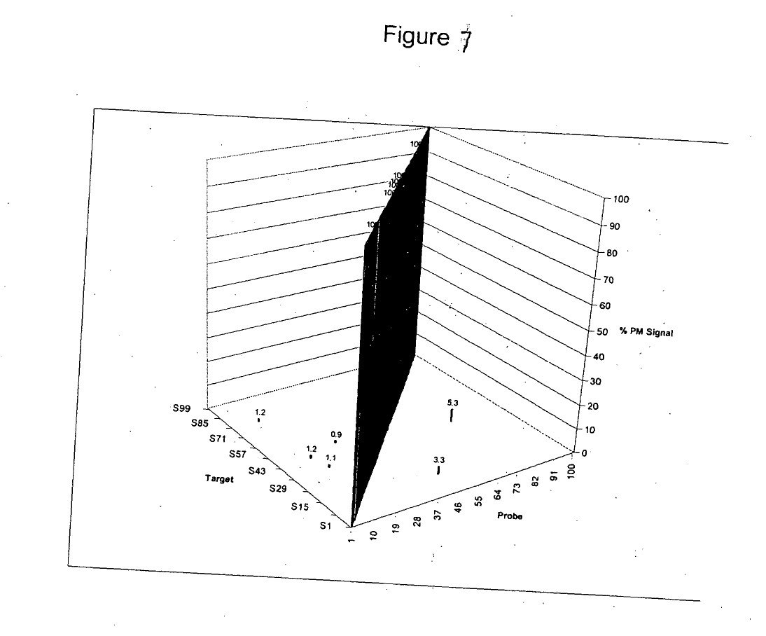

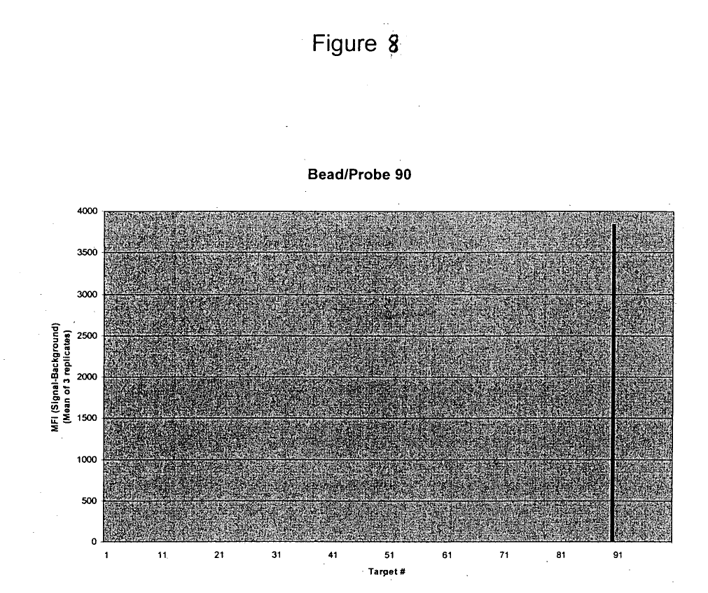

FIG. 7 is a three dimensional representation showing cross-hybridization observed for the sequences of FIG. 6 as described in Example 4. The results shown in FIG. 6 are reproduced along the diagonal of the drawing;

FIG. 8 is illustrative of the results obtained for an individual target (Table II, SEQ ID No: 90, target No. 90) when exposed to the 100 probes of Example 4. The MFI for each bead is plotted.

### DETAILED DESCRIPTION OF THE INVENTION

The invention provides a method for sorting complex mixtures of molecules by the use of families of oligonucleotide sequence tags. The families of oligonucleotide sequence tags are designed so as to provide minimal cross hybridization during the sorting process. Thus any sequence within a family of sequences will not cross hybridize with any other sequence derived from that family under appropriate hybridization conditions known by those skilled in the art. The invention is particularly useful in highly parallel processing of analytes.

Families of Oligonucleotide Sequence Tags

The present invention includes a family of 24mer polynucleotides, that have been demonstrated to be minimally cross-hybridizing with each other. This family of polynucleotides is thus useful as a family of tags, and their complements as tag complements.

The oligonucleotide sequences that belong to families of sequences that do not exhibit cross hybridization behavior can be derived by computer programs (described in U.S. Provisional Patent Application No. 60/181,563 filed Feb. 10, 2000). The programs use a method of generating a maximum number of minimally cross-hybridizing polynucleotide sequences that can be summarized as follows. First, a set of sequences of a given length are created based on a given number of block elements. Thus, if a family of polynucleotide sequences 24 nucleotides (24mer) in length is desired from a set of 6 block elements, each element comprising 4 nucleotides, then a family of 24mers is generated considering all positions of the 6 block elements. In this case, there will be 6⁶ (46,656) ways of assembling the 6 block elements to generate all possible polynucleotide sequences 24 nucleotides in length.

Constraints are imposed on the sequences and are expressed as a set of rules on the identities of the blocks such that homology between any two sequences will not exceed the degree of homology desired between these two sequences. All polynucleotide sequences generated which obey the rules are saved. Sequence comparisons are performed in order to generate an incidence matrix. The incidence matrix is presented as a simple graph and the sequences with the desired property of being minimally cross hybridizing are found from a clique of the simple graph, which may have multiple cliques. Once a clique containing a suitably large number of sequences is found, the sequences are experimentally tested to determine if it is a set of minimally cross hybridizing sequences. This method has been used to obtain the 100 non cross-hybridizing tags of Table I that are the subject of Example 1.

The method includes a rational approach to the selection of groups of sequences that are used to describe the blocks. For example there are n⁴ different tetramers that can be obtained from n different nucleotides, non-standard bases or analogues thereof. In a more preferred embodiment there are 44 or 256 possible tetramers when natural nucleotides are used. More preferably 81 possible tetramers when only 3 bases are used A, T and G. Most preferably 32 different tetramers when all sequences have only one G.

Block sequences can be composed of a subset of natural bases most preferably A, T and G. Sequences derived from blocks that are deficient in one base possess useful characteristics, for example, in reducing potential secondary structure formation or reduced potential for cross hybridization with nucleic acids in nature. Sets of block sequences that are most preferable in constructing families of non cross hybridizing tag sequences should contribute approximately equivalent stability to the formation of the correct duplex as all other block sequences of the set. This should provide tag sequences that behave isothermally. This can be achieved, for example, by maintaining a constant base composition for all block sequences such as one G and three A's or T's for each block sequence. Preferably, non-cross hybridizing sets of block sequences will be comprised from blocks of sequences that are isothermal. The block sequences should be different from each other by at least one mismatch. Guidance for selecting such sequences is provided by methods for selecting primer and or probe sequences that can be found in published techniques (Robertson et al., Methods Mol Biol; 98:121-54 (1998); Rychlik et al, Nucleic Acids Research, 17:8543-8551 (1989); Breslauer et al., Proc Natl Acad. Sci., 83:3746-3750 (1986)) and the like. Additional sets of sequences can be designed by extrapolating on the original family of non cross hybridizing sequences by simple methods known to those skilled in the art.

A preferred family of 100 tags is shown as SEQ ID NOs:1 to 100 in Table I. Characterization of the family of 100 sequence tags was performed to determine the ability of these sequences to form specific duplex structures with their complementary sequences and to assess the potential for cross hybridization. The 100 sequences were synthesized and spotted onto glass slides where they were coupled to the surface by amine linkage. Complementary tag sequences were Cy3-labeled and hybridized individually to the array containing the family of 100 sequence tags. Formation of duplex structures was detected and quantified for each of the positions on the array. Each of the tag sequences performed as expected, that is the perfect match duplex was formed in the absence of significant cross hybridization under stringent hybridization conditions. The results of a sample hybridization are shown in FIG. 1. FIG. 1a shows the hybridization pattern seen when a microarray containing all 100 probes was hybridized with the target complementary to probe 181234. The 4 sets of paired spots correspond to the probe complementary to the target. FIG. 1b shows the pattern seen when a similar array was hybridized with a mix of all 100 targets. These results indicate that the family of sequences which is the subject of this patent can be used as a family of non-cross hybridizing (tag) sequences.

The family of 100 non-cross-hybridizing sequences can be expanded by incorporating additional tetramer sequences that are used in constructing further 24mer oligonucleotides. In one example, four additional words were included in the generation of new sequences to be considered for inclusion as non-cross talkers in a family of sequences that were obtained from the above method using 10 tetramers. In this case, the four additional words were selected to avoid potential homologies with all potential combinations of other words: YYXW (TTAG); WYYX (GTTA); XYXW (ATAG) and WYYY (GTTT). The total number of sequences containing six words using the 14 possible words is 14⁶ or 7,529,536. These sequences were screened to eliminate sequences that contain repetitive regions that present potential hybridization problems such as four or more of a similar base (e.g., AAAA or TTTT) or pairs of G's. Each of these sequences was compared to the sequence set of the original family of 100 non-cross-hybridizing sequences (SEQ ID NOs:1 to 100). Any new sequence that contained a minimal threshold of homology (that does not include the use of insertions or deletions) such as 15 or more matches with any of the original family of sequences was eliminated. In other words, if it was possible to align a new sequence with one or more of the original 100 sequences so as to obtain a maximum simple homology of 15/24 or more, the new sequence was dropped. “Simple homology” between a pair of sequences is defined here as the number of pairs of nucleotides that are matching (are the same as each other) in a comparison of two aligned sequences divided by the total number of potential matches. “Maximum simple homology” is obtained when two sequences are aligned with each other so as to have the maximum number of paired matching nucleotides. In any event, the set of new sequences so obtained was referred to as the “candidate sequences”. One of the candidate sequences was arbitrarily chosen and referred togas sequence 101. All the candidate sequences were checked against sequence 101, and sequences that contained 15 or more non-consecutive matches (i.e., a maximum simple homology of 15/24 (62.5%) or more were eliminated. This results in a smaller set of candidate sequences from which another sequence is selected that is now referred to as sequence 102. The smaller set of candidate sequences is now compared to sequence 102 eliminating sequences that contained 15 or more non-consecutive matches and the process is repeated until there are no candidate sequences remaining. Also, any sequence selected from the candidate sequences is eliminated if it has 13 or more consecutive matches with any other previously selected candidate sequence.

The additional set of 73tag sequences so obtained (SEQ ID NOs:101 to 173 of Table 1) is composed of sequences that when compared to any of SEQ ID NOs:1 to 100 of Table I have no greater similarity than the sequences of the original 100 sequence tags of Table I. The sequence set as derived from the original family of non cross hybridizing sequences, SEQ ID NOs:1 to 173 of Table 1, are expected to behave with similar hybridization properties to the sequences having SEQ ID NOs:1 to 100 since it is understood that sequence similarity correlates directly with cross hybridization (Southern et al., Nat. Genet.; 21, 5-9: 1999).

The set of 173 24mer oligonucleotides were expanded to include those having SEQ ID NOs:174 to 210 as follows. The 4mers WXYW, XYXW, WXXW, WYYW, XYYX, YXYX, YXXY and XYXY where W=G, X=A, and Y=U/T were used in combination with the fourteen 4mers used in the generation of SEQ ID NOs:1 to 173 to generate potential 24-base oligonucleotides. Excluded from the set were those containing the sequence patterns GG, AAAA and TTTT. To be included in the set of additional 24mers, a sequence also had to have at least one of the 4mers containing two G's: WXYW (GATG), WYXW (GTAG), WXXW (GAAG), WYYW (GTTG) while also containing exactly six G's. Also required for a 24mer to be included was that there be at most six bases between every neighboring pair of G's. Another way of putting this is that there are at most six non-G's between any two G's. Also, each G nearest the 5′-end of its oligonucleotide (the left-hand side as written in Table I) was required to occupy one of the first to seventh positions (counting the 5′-terminal position as the first position.) A set of candidate sequences was obtained by eliminating any new sequence that was found to have a maximum simple homology of 16/24 or more with any of the previous set of 173 oligonucleotides (Table 1, SEQ ID NOs:1 to 0.173). As above, an arbitrary 174^{th} sequence was chosen and candidate sequences eliminated by comparison therewith. In this case the permitted maximum degree of simple homology was 16/24. A second sequence was also eliminated if there were ten consecutive matches between the two (i.e., it was notionally possible to generate a phantom sequence containing a sequence of 10 bases that is identical to a sequence in each of the sequences being compared). A second sequence was also eliminated if it was possible to generate a phantom sequence 20 bases in length or greater.

A property of the polynucleotide sequences shown in Table I is that the maximum block homology between any two sequences is never greater than 66⅔ percent. This is because the computer algorithm by which the sequences were initially generated was designed to prevent such an occurrence. It is within the capability of a person skilled in the art, given the family of sequences of Table I, to modify the sequences, or add other sequences while largely retaining the property of minimal-cross hybridization which the polynucleotides of Table I have been demonstrated to have.

There are 210 polynucleotide sequences given in Table I. Since all 210 of this family of polynucleotides can work with each other as a minimally cross-hybridizing set, then any plurality of polynucleotides that is a subset of the 210 can also act as a minimally cross-hybridizing set of polynucleotides. An application in which, for example, 30 molecules are to be sorted using a family of polynucleotide tags and tag complements could thus use any group of 30 sequences shown in Table I. This is not to say that some subsets may be found in practical sense to be more preferred than others. For example, it may be found that a particular subset is more tolerant of a wider variety of conditions under which hybridization is conducted before the degree of cross-hybridization becomes unacceptable.

It may be desirable to use polynucleotides that are shorter in length than the 24 bases of those in Table I. A family of subsequences (i.e., subframes of the sequences illustrated) based on those contained in Table I having as few as 10 bases per sequence could be chosen, so long as the subsequences are chosen to retain homological properties between any two of the sequences of the family important to their non cross-hybridization.

The selection of sequences using this approach would be amenable to a computerized process. Thus for example, a string of 10 contiguous bases of the first 24mer of Table I could be selected: GATTTGTATTGATTGAGATTAAAG.

A string of contiguous bases from the second 24mer could then be selected and compared for maximum homology against the first chosen sequence: TGATTGTAGTATGTATTGATAAAG

Systematic pairwise comparison could then be carried out to determine if the maximum homology requirement of 66⅔ percent is violated:

| | |
|---|---|
| Alignment | Matches |

| | |
|---|---|
| GATTTGTATT | 1 |
| ATTGATAAAG | |
| GATTTGTATT | 0 |
| ATTGATAAAG | |
| GATTTGTATT | 1 |
| ATTGATAAAG | |
| GATTTGTATT | 1 |
| ATTGATAAAG | |
| GATTTGTATT | 1 |
| ATTGATAAAG | |
| GATTTGTATT | 1 |
| ATTGATAAAG | |
| GATTTGTATT | 3 |
| ATTGATAAAG | |
| GATTTGTATT | 1 |
| ATTGATAAAG | |
| GATTTGTATT | 2 |
| ATTGATAAAG | |
| GATTTGTATT | 2 |
| ATTGATAAAG | |
| GATTTGTATT | 5 (*) |
| ATTGATAAAG | |
| GATTTGTATT | 3 |
| ATTGATAAAG | |
| GATTTGTATT | 3 |
| ATTGATAAAG | |
| GATTTGTATT | 2 |
| ATTGATAAAG | |
| GATTTGTATT | 1 |
| ATTGATAAAG | |
| GATTTGTATT | 1 |
| ATTGATAAAG | |
| GATTTGTATT | 3 |
| ATTGATAAAG | |
| GATTTGTATT | 1 |
| ATTGATAAAG | |
| GATTTGTATT | 0 |
| ATTGATAAAG | |

As can be seen, the maximum homology between the two selected subsequences is 50 percent (5 matches out of the total length of 10), and so these two sequences are compatible with each other.

A 10mer subsequence can be selected from the third 24mer sequence of Table I, and pairwise compared to each of the first two 10mer sequences to determine its compatability therewith, etc. and in this way a family of 10mer sequences developed.

It is within the scope of this invention, to obtain families of sequences containing 11mer, 12mer, 13mer, 14mer, 15mer, 16mer, 17mer, 18mer, 19mer, 20mer, 21mer, 22mer and 23mer sequences by analogy to that shown for 10mer sequences.

It may be desirable to have a family of sequences in which there are sequences greater in length than the 24mer sequences shown in Table I. It is within the capability of a person skilled in the art, given the family of sequences shown in Table I, to obtain such a family of sequences. One possible approach would be to insert into each sequence at one or more locations a nucleotide, non natural base or analogue such that the longer sequence should not have greater similarity than any two of the original non cross hybridizing sequences of Table I and the addition of extra bases to the tag sequences should not result in a major change in the thermodynamic properties of the tag sequences of that set for example the GC content must be maintained between 10%-40% with a variance from the average of 20%. This method of inserting bases could be used to obtain a family of sequences up to 40 bases long.

Given a particular family of sequences that can be used as a family of tags (or tag complements), e.g., those of Table I or Table II, or the combined sequences of these two tables, a skilled person will readily recognize variant families that work equally as well.

Again taking the sequences of Table I for example, every T could be converted to an A and vice versa and no significant change in the cross-hybridization properties would be expected to be observed. This would also be true if every G were converted to a C.

Also, all of the sequences of a family could be taken to be constructed in the 5′-3′ direction, as is the convention, or all of the constructions of sequences could be in the opposition direction (3′-5′).

There are additional modifications that can be carried out. For example, C has not been used in the family of sequences. Substitution of C in place of one or more T's of a particular sequence would yield a sequence that is at least as low in homology with every other sequence of the family as the particular sequence chosen to be modified was. It is thus possible to substitute C in place of one or more T's in any of the sequences shown in Table I. Analogously, substituting of C in place of one or more A's is possible, or substituting C in place of one or T's is possible.

It is preferred that the sequences of a given family are of the same, or roughly the same length. Preferably, all the sequences of a family of sequences of this invention have a length that is within five bases of the base-length of the average of the family. More preferably, all sequences are within four bases of the average base-length. Even more preferably, all or almost all sequences are within three bases of the average base-length of the family. Better still, all or almost all sequences have a length that is within two of the base-length of the average of the family.

It is also possible for a person skilled in the art to derive sets of sequences from the family of sequences that is the subject of this patent and remove sequences that would be expected to have undesirable hybridization properties.

Methods for Synthesis of Oligonucleotide Families

Preferably oligonucleotide sequences of the invention are synthesized directly by standard phosphoramidite synthesis approaches and the like (Caruthers et al, Methods in Enzymology; 154, 287-313: 1987; Lipshutz et al, Nature Genet.; 21, 20-24: 1999; Fodor et al, Science; 251, 763-773: 1991). Alternative chemistries involving non natural bases such as peptide nucleic acids or modified nucleosides that offer advantages in duplex stability may also be used (Hacia et al; Nucleic Acids Res; 27: 4034-4039, 1999; Nguyen et al, Nucleic Acids Res.; 27, 1492-1498: 1999; Weiler et al, Nucleic Acids Res.; 25, 2792-2799:1997). It is also possible to synthesize the oligonucleotide sequences of this invention with alternate nucleotide backbones such as phosphorothioate or phosphoroamidate nucleotides. Methods involving synthesis through the addition of blocks of sequence in a step wise manner may also be employed (Lyttle et al, Biotechniques, 19: 274-280 (1995). Synthesis may be carried out directly on the substrate to be used as a solid phase support for the application or the oligonucleotide can be cleaved from the support for use in solution or coupling to a second support.

Solid Phase Supports

There are several different solid phase supports that can be used with the invention. They include but are not limited to slides, plates, chips, membranes, beads, microparticles and the like. The solid phase supports can also vary in the materials that they are composed of including plastic, glass, silicon, nylon, polystyrene, silica gel, latex and the like. The surface of the support is coated with the complementary sequence of the same.

In preferred embodiments, the family of tag complement sequences are derivatized to allow binding to a solid support. Many methods of derivatizing a nucleic acid for binding to a solid support are known in the art (Hermanson G., Bioconjugate Techniques; Acad. Press: 1996). The sequence tag may be bound to a solid support through covalent or non-covalent bonds (Iannone et al, Cytometry; 39: 131-140, 2000; Matson et al, Anal. Biochem.; 224: 110-106, 1995; Proudnikov et al, Anal Biochem; 259: 34-41, 1998; Zammatteo et al, Analytical Biochemistry; 280:143-150, 2000). The sequence tag can be conveniently derivatized for binding to a solid support by incorporating modified nucleic acids in the terminal 5′ or 3′ locations.

A variety of moieties useful for binding to a solid support (e.g., biotin, antibodies, and the like), and methods for attaching them to nucleic acids, are known in the art. For example, an amine-modified nucleic acid base (available from, eg., Glen Research) may be attached to a solid support (for example, Covalink-NH, a polystyrene surface grafted with secondary amino groups, available from Nunc) through a bifunctional crosslinker (e.g., bis(sulfosuccinimidyl suberate), available from Pierce). Additional spacing moieties can be added to reduce steric hindrance between the capture moiety and the surface of the solid support.

Attaching Tags to Analytes for Sorting

A family of oligoucleotide tag sequences can be conjugated to a population of analytes most preferably polynucleotide sequences in several different ways including but not limited to direct chemical synthesis, chemical coupling, ligation, amplification, and the like. Sequence tags that have been synthesized with primer sequences can be used for enzymatic extension of the primer on the target for example in PCR amplification.

Detection of Single Nucleotide Polymorphisms Using Primer Extension

There are a number of areas of genetic analysis where families of non cross hybridizing sequences can be applied including disease dagnosis, single nucleotide polymorphism analysis, genotyping, expression analysis and the like. One such approach for genetic analysis referred to as the primer extension method (also known as Genetic Bit Analysis (Nikiforov et al, Nucleic Acids Res.; 22, 4167-4175: 1994; Head et al Nucleic Acids Res.; 25, 5065-5071: 1997)) is an extremely accurate method for identification of the nucleotide located at a specific polymorphic site within genomic DNA. In standard primer extension reactions, a portion of genomic DNA containing a defined polymorphic site is amplified by PCR using primers that flank the polymorphic site. In order to identify which nucleotide is present at the polymorphic site, a third primer is synthesized such that the polymorphic position is located immediately 3′ to the primer. A primer extension reaction is set up containing the amplified DNA, the primer for extension, up to 4 dideoxynucleoside triphosphates, each labelled with a different fluorescent dye and a DNA polymerase such as the Klenow subunit of DNA Polymerase 1. The use of dideoxy nucleotides ensure that a single base is added to the 3′ end of the primer, a site corresponding to the polymorphic site. In this way the identity of the nucleotide present at a specific polymorphic site can be determined by the identity of the fluorescent dye-labelled nucleotide that is incorporated in each reaction. One major drawback to this approach is its low throughput. Each primer extension reaction is carried out independently in a separate tube.

Universal sequences can be used to enhance the throughput of primer extension assay as follows. A region of genomic DNA containing multiple polymorphic sites is amplified by PCR. Alternately, several genomic regions containing one or more polymorphic sites each are amplified together in a multiplexed PCR reaction. The primer extension reaction is carried out as described above except that the primers used are chimeric, each containing a unique universal tag at the 5′ end and the sequence for extension at the 3′ end. In this way, each gene-specific sequence would be associated with a specific universal sequence. The chimeric primers would be hybridized to the amplified DNA and primer extension carried out as described above. This would result in a mixed pool of extended primers, each with a specific fluorescent dye characteristic of the incorporated nucleotide. Following the primer extension reaction, the mixed extension reactions are hybridized to an array containing probes that are reverse complements of the universal sequences on the primers. This would segregate the products of a number of primer extension reactions into discrete spots. The fluorescent dye present at each spot would then identify the nucleotide incorporated at each specific location.

Kits Using Families of Tag Sequences

The families of non cross-hybridizing sequences may be provided in kits for use in for example genetic analysis. Such kits include at least one set of non cross hybridizing sequences in solution or on a solid support. Preferably the sequences are attached to microparticles and are provided with buffers and reagents that are appropriate for the application. Reagents may include enzymes, nucleotides, fluorescent labels and the like that would be required for specific applications. Instructions for correct use of the kit for a given application will be provided.

### EXAMPLES

### Example 1

Demonstrate Non Cross Talk Behavior

One hundred oligonucleotide probes corresponding to a family of non-cross talking oligonucleotides from Table I were synthesized by Integrated DNA Technologies (IDT, Coralville Iowa). These oligonucleotides incorporated a C₆ aminolink group coupled to the 5′ end of the oligo through a C₁₈ ethylene glycol spacer. These probes were used to prepare microarrays as follows. The probes were resuspended at a concentration of 50 μM in 150 mM NaPO4, pH 8.5. The probes were spotted onto the surface of a SuperAldehyde slide (Telechem Int., Sunnyvale Calif.) using and SDDC-II microarray spotter (ESI, Toronto Ont). The spots formed were approximately 120 FM in diameter with 200 μM centre-to-centre spacing. Each probe was spotted 8 times on each microarray. Following spotting, the arrays were processed essentially as described by the slide manufacturer. Briefly, the arrays were treated with 67 mM sodium borohydride in PBS/EtOH (3:1) for 5 minutes then washed with 4 changes of 0.1% SDS. The arrays were not boiled.

One hundred labelled oligonucleotide targets were also synthesized by IDT. The sequence of these targets corresponded to the reverse complement of the 100 probe sequences. The targets were labelled at the 5′ end with Cy3.

Each Cy3-labeled target oligonucleotide was hybridized separately to two microarrays each of which contained all 100 oligonucleotide probes. Hybridizations were carried out at 42° C. for 2 hours in a 40 ll reaction and contained 40 nM of the labelled target suspended in 10 mM Tris HCl, pH 8.3, 50 mM KCl, 0.1% Tween 20. These are low stringency hybridization conditions designed to provide a rigorous test of the performance of the family of non-cross hybridizing sequences. Hybridizations were carried out by depositing the hybridization solution on a clean cover slip then carefully positioning the microarray slide over the cover slip in order to avoid bubbles. The slide was then inverted and transferred to a humid chamber for incubation. Following hybridization, the cover slip was removed and the microarray was washed in hybridization buffer for 15 minutes at room temperature. The slide was then dried by brief centrifugation.

Hybridized microarrays were scanned using a ScanArray Lite (GSI-Lumonics, Billerica Mass.). The laser power and photomultiplier tube voltage used for scanning each hybridized microarray were optimized in order to maximize the signal intensity from the spots representing the perfect match.

The results of a sample hybridization are shown in FIG. 1. FIG. 1a shows the hybridization pattern seen when a microarray containing all 100 probes was hybridized with the target complementary to probe 181234. The 4 sets of paired spots correspond to the probe complementary to the target. FIG. 1b shows the pattern seen when a similar array was hybridized with a mix of all 100 targets.

### Example 2

### Tag Sequences Used in Sorting Polynucleotides

The family of non cross hybridizing sequence tags or a subset thereof can be attached to oligonucleotide probe sequences during synthesis and used to generate amplified probe sequences. In order to test the feasibility of PCR amplification with non cross hybridizing sequence tags and subsequently addressing each respective sequence to its appropriate location on two-dimensional or bead arrays, the following experiment was devised. A 24mer tag sequence was connected in a 5′-3′ specific manner to a p53 exon specific sequence (20mer reverse primer). The connecting p53 sequence represented the inverse complement of the nucleotide gene sequence. To facilitate the subsequent generation of single stranded DNA post-amplification the tag-Reverse primer was synthesized with a phosphate modification (PO₄) on the 5′-end. A second PCR primer was also generated for each desired exon, which represented the Forward (5′-3′) amplification primer. In this instance the Forward primer was labeled with a 5′-biotin modification to allow detection with Cy3-avidin or equivalent.

A practical example of the aforementioned description is as follows: For exon 1 of the human p53 tumor suppressor gene sequence the following tag-Reverse primer was generated:

```
                                                    222087 222063
5′-P04-GATTGTAAGATTTGATAAAGTGTA-TCCAGGGAAGCGTGTCACCGTCGT-3′
            Tag Sequence # 3 Exon 1 Reverse
```

The numbering above the Exon-1 reverse primer represents the genomic nucleotide positions of the indicated bases. The corresponding Exon-1 Forward primer sequence is as follows:

221873 221896
5′-Biotin-TCATGGCGACTGTCCAGCTTTGTG-3′

In combination these primers will amplify a product of 214 bp plus a 24 bp tag extension yielding a total size of 0.238 bp. Once amplified, the PCR product was purified using a QIAquick PCR purification kit and the resulting DNA was quantified. To generate single stranded DNA the DNA was subjected to—exonuclease digestion thereby resulting in the exposure of a single stranded sequence (anti-tag) complementary to the tag-sequence covalently attached to the solid phase array. The resulting product was heated to 95° C. for 5 minutes and then directly applied to the array at a concentration of 10-50 nM. Following hybridization and concurrent sorting, the tag-Exon 1 sequences were visualized using Cy3-streptavidin. In addition to direct visualization of the biotinylated product, the product itself can now act as a substrate for further analysis of the amplified region, such as SNP detection and haplotype determination.

The present invention also includes a family of 1168 24mer polynucleotides that have been demonstrated to be minimally cross-hybridizing with each other. This family of polynucleotides is thus useful as a family of tags, and their complements as tag complements.

In order to be considered for inclusion into the family, a sequence had to satisfy a certain number of rules regarding its composition. For example, repetitive regions that present potential hybridization problems such as four or more of a similar base (e.g., AAAA or TTTT) or pairs of Gs were forbidden. Another rule is that each sequence contains exactly six Gs and no Cs, in order to have sequences that are more or less isothermal. Also required for a 24mer to be included is that there must be at most six bases between every neighboring pair of Gs. Another way of putting this is that there are at most six non-Gs between any two consecutive Gs. Also, each G nearest the 5′-end (resp. 3′-end) of its oligonucleotide (the left-hand (resp. right-hand) side as written in Table II) was required to occupy one of the first to seventh positions (counting the 5′-terminal (resp. 3′-terminal) position as the first position.)

The process used to design families of sequences that do not exhibit cross-hybridization behavior is illustrated generally in FIG. 5). Depending on the application for which these families of sequences will be used, various rules are designed. A certain number of rules can specify constraints for sequence composition (such as the ones described in the previous paragraph). The other rules are used to judge whether two sequences are too similar. Based on these rules, a computer program can derive families of sequences that exhibit minimal or no cross-hybridization behavior. The exact method used by the computer program is not crucial since various computer programs can derive similar families based on these rules. Such a program is for example described in international patent application No. PCT/CA 01/00141 published under WO 01/59151 on Aug. 16, 2001. Other, programs can use different methods, such as the ones summarized below.

A first method of generating a maximum number of minimally cross-hybridizing polynucleotide sequences starts with any number of non-cross-hybridizing sequences, for example just one sequence, and increases the family as follows. A certain number of sequences is generated and compared to the sequences already in the family. The generated sequences that exhibit too much similarity with sequences already in the family are dropped. Among the “candidate sequences” that remain, one sequence is selected and added to the family. The other candidate sequences are then compared to the selected sequence, and the ones that show too much similarity are-dropped. A new sequence is selected from the remaining candidate sequences, if any, and added to the family, and soon until there are no candidate sequences left. At this stage, the process can be repeated (generating a certain number of sequences and comparing them to the sequences in the family, etc.) as often as desired. The family obtained at the end of this method contains only minimally cross-hybridizing sequences.

A second method of generating a maximum number of minimally cross-hybridizing polynucleotide sequences starts with a fixed-size family of polynucleotide sequences. The sequences of this family can be generated randomly or designed by some other method. Many sequences in this family may not be compatible with each other, because they show too much similarity and are not minimally cross-hybridizing. Therefore, some sequences need to be replaced by new ones, with less similarity. One way to achieve this consists of repeatedly replacing a sequence of the family by the best (that is, lowest similarity) sequence among a certain number of (for example, randomly generated) sequences that are not part of the family. This process can be repeated until the family of sequences shows minimal similarity, hence minimal cross-hybridizing, or until a set number of replacements has occurred. If, at the end of the process, some sequences do not obey the similarity rules that have been set, they can be taken out of the family, thus providing a somewhat smaller family that only contains minimally cross-hybridizing sequences. Some additional rules can be added to this method in order to make it more efficient, such as rules to determine which sequence will be replaced.

Such methods have been used to obtain the 1168 non-cross-hybridizing tags of Table II that are also the subject of this patent application.

One embodiment of the invention is a composition comprising molecules for use as tags or tag complements wherein each molecule comprises an oligonucleotide selected from a set of oligonucleotides based on the group of sequences set out in Table IIA, wherein each of the numeric identifiers 1 to 3-(see the Table) is a nucleotide base selected to be different from the others of 1 to 3. According to this embodiment, several different families of specific sets of oligonucleotide sequences are described, depending upon the assignment of bases made to the numeric identifiers 1 to 3.

The sequences contained in Table II have a mathematical relationship to each other, described as follows.

Let S and T be two DNA sequences of lengths s and t respectively. While the term “alignment” of nucleotide sequences is widely used in the field of biotechnology, in the context of this invention the term has a specific meaning illustrated here. An alignment of S and T is a 2xp matrix A (with p 2 s and p≧t) such that the first (or second) row of A contains the characters of S (or T respectively) in order, interspersed with p−s (or p−t respectively) spaces. It assumed that no column of the alignment matrix contains two spaces, i.e., that any alignment in which a column contains two spaces is ignored and not considered here. The columns containing the same base in both rows are called matches, while the columns containing different bases are called mismatches. Each column of an alignment containing a space in its first row is called an insertion and each column containing a space in its second row is called a deletion while a column of the alignment containing a space in either row is called an indel. Insertions and deletions within a sequence are represented by the character ‘-’. A gap is a continuous sequence of spaces in one of the rows (that is neither immediately preceded nor immediately followed by another space in the same row), and the length of a gap is the number of spaces in that gap. An internal gap is one in which its first space is preceded by a base and its last space is followed by a base and an internal indel is an belonging to an internal gap. Finally, a block is a continuous sequence of matches (that is neither immediately preceded nor immediately followed by another match), and the length of a block is the number of matches in that block. In order to illustrate these definitions, two sequences S=TGATCGTAGCTACGCCGCG (of length s=19; SEQ ID NO:1169) and T=CGTACGATTGCAACGT (of length t=16, SEQ ID NO:1170) are considered. Exemplary alignment R₁ of S and T (with p=23) is:

Alignment R₁:

| — | — | — | — | T | G | A | T | C | G | T | A | G | C | T | A | C | G | C | C | G | C | G |
|---|---|---|---|---|---|---|---|---|---|---|---|---|---|---|---|---|---|---|---|---|---|---|
| C | G | T | A | C | G | A | T | — | — | T | — | G | C | A | A | C | G | T | — | — | — | — |
| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |

Columns 1 to 4, 9, 10, 12 and 20 to 23 are indels, columns 6, 7, 8, 11, 13, 14, 16, 17 and 18 are matches, and columns 5, 15 and 19 are mismatches. Columns 9 and 10 form a gap of length 2, while columns 16 to 18 form a block of length 3. Columns 9, 10 and 12 are internal indels.

A score is assigned to the alignment A of two sequences by assigning weights to each of matches, mismatches and gaps as follows:
the reward for a match m,
the penalty for a mismatch mm,
the penalty for opening a gap og
the penalty for extending a gap eg.
   Once these values are set, a score to each column of the alignment is assigned according to the following rules:
1. assign 0 to each column preceding the first match and to each column following the last match.
2. for each of the remaining columns, assign m if it is a match, mm if it is a mismatch, -og-eg if it is the first indel of a gap, -eg if it is an indel but not the first indel of a gap.
   The score of the alignment A is the sum of the scores of its columns. An alignment is said to be of maximum score if no other alignment of the same two sequences has a higher score (with the same values of m, mm, og and eg). A person knowledgeable in the field will recognize this method of scoring an alignment as scoring a local (as opposed to global) alignment with affine gap penalties (that is, gap penalties that can distinguish between the first indel of a gap and the other indels). It will be appreciated that the total number of indels that open a gap is the same as the total number of gaps and that an internal indel is not one of those assigned a 0 in rule (1) above. It will also be noted that foregoing rule (1) assigns a 0 for non-internal mismatches. An internal mismatch is a mismatch that is preceded and followed (not necessarily immediately) by a match.

As an illustration, if the values of m, mm, og and eg are set to 3, 1, 2 and 1 respectively, alignment R₁ has a score of. 19, determined as shown below:

Scoring of Alignment R₁

| — | — | — | — | T | G | A | T | C | G | T | A | G | C | T | A | C | G | C | C | G | C | G |
|---|---|---|---|---|---|---|---|---|---|---|---|---|---|---|---|---|---|---|---|---|---|---|
| C | G | T | A | C | G | A | T | — | — | T | — | G | C | A | A | C | G | T | — | — | — | — |
| 0 | 0 | 0 | 0 | 0 | 3 | 3 | 3 | −3 | −1 | 3 | −3 | 3 | 3 | −1 | 3 | 3 | 3 | 0 | 0 | 0 | 0 | 0 |

Note that for two given sequences S and T, there are numerous alignments. There are often several alignments of maximum score.

Based on these alignments, five sequence similarity measures are defined as follows. For two sequences S and T, and weights {m, mm, og, eg}:
M1 is the maximum number of matches over all alignments free of internal indels;
M2 is the maximum length of a block over all alignments;
M3 is the maximum number of matches over all alignments of maximum score;
M4 is the maximum sum of the lengths of the longest two blocks over all alignments of maximum score;
M5 is the maximum sum of the lengths of all the blocks of length at least 3, over all alignments of maximum score.
   Notice that, by definition, the following inequalities between these similarity measures are obtained: M4≦M3 and M5≦M3. Also, in order to determine M2 it is sufficient to determine the maximum length of a block over all alignments free of internal indels. For two given sequences, the values of M3 to M5 can vary depending on the values of the weights {m, mm, og, eg}, but not M1 and M2.

For weights {3, 1, 2, 1}, the illustrated alignment is not a maximum score alignment of the two example sequences. But for weights {6, 6, 0, 6} it is; hence this alignment shows that for these two example sequences, and weights {6, 6, 0, 6}, M2≧3, M3≧9, M4≧6 and M5≧6. In order to determine the exact values of M1 to M5, all the necessary alignments need to be considered. M1 add M2 can be found by looking at the s+t−1 alignments free of internal indels, where s and t are the lengths of the two sequences considered. Mathematical tools known as dynamic programming can be implemented on a computer and used to determine M3 to M5 in a very quick way. Using a computer program to do these calculations, it was determined that:
with the weights {6, 6, 0, 6}, M1=8, M2=4, M3=10, M4=6 and M5=6;
with the weights {3, 1, 2, 1}, M1=8, M2=4, M3=10, M4=6 and M5=4.
   According to the preferred embodiment of this invention, two sequences S and T each of length 24 are too similar if at least one of the following happens:
M1>16 or
M2>13 or
M3>20 or
M4>16 or
M5>19
   when using either weights {6, 6, 0, 6}, or {6, 6, 5, 1}, or {6, 2, 5, 1}, or {6, 6, 6, 0}. In other words, the five similarity measures between S and T are determined for each of the above four sets of weights, and checked against these thresholds (for a total of 20 tests).

The above thresholds of 16, 13, 20, 16 and 19, and the above sets of weights, were used to obtain the sequences listed in Table I. Additional sequences can thus be added to those of Table I as long as the above alignment rules are obeyed for all sequences.

It is also possible to alter thresholds M1, M2, etc., while remaining within the scope of this invention. It is thus possible to substitute or add sequences to those of Table II, or more generally to those of Table IIA to obtain other sets of sequences that would also exhibit reasonably low cross-hybridization. More specifically, a set of 24mer sequences in which there are no two sequences that are too similar, where too similar is defined as:
M1>19 or
M2>17 or
M3>21 or
M4>18 or
M5>20
   when using either weights {6, 6, 0, 6}, or {6, 6, 5, 1}, or {6, 2, 5, 1}, or {6, 6, 6, 0}, would also exhibit low cross-hybridization. Reducing any of the threshold values provides sets of sequences with even lower cross-hybridization. Alternatively, ‘too similar’ can also be defined as:
M1>19 or
M2>17 or
M3>21 or
M4>18 or
M5>20
   when using either weights {3, 1, 2, 1}. Alternatively, other combinations of weights will lead to sets of sequences with low cross-hybridization.

Notice that using weights {6, 6, 0, 6} is equivalent to using weights {1, 1, 0, 1}, or weights {2, 2, 0, 2}, . . . (that is, for any two sequences, the values of M1 to M5 are exactly the same whether weights {6, 6, 0, 6} or {1, 1, 0, 1} or {2, 2, 0, 2} or any other multiple of {1, 1, 0, 1} is used).

When dealing with sequences of length other than 24, or sequences of various lengths, the definition of similarity can be adjusted. Such adjustments are obvious to the persons skilled in the art. For example, when comparing a sequence of length L1 with a sequence of length L2 (with L1<L2), they can be considered as too similar when
M1>19/24×L1
M2>17/24×L1
M3>21/24×L1
M4>18/24×L1
M5>20/24×L1
   when using either weights {6, 6, 0, 6}, or {6, 6, 5, 1}, or {6, 2, 5, 1} or {6, 6, 6, 0}.

Polynucleotide sequences can be composed of a subset of natural bases most preferably A, T and G. Sequences that are deficient in one base possess useful characteristics, for example, in reducing potential secondary structure formation or reduced potential for cross hybridization with nucleic acids in nature. Also, it is preferable to have tag sequences that behave isothermally. This can be achieved for example by maintaining a constant base composition for all sequences such as six Gs and eighteen As or Ts' for each sequence. Additional sets of sequences can be designed by extrapolating on the original family of non-cross-hybridizing sequences by simple methods known to those skilled in the art.

In order to validate the sequence set, a subset of sequences from the family of 1168 sequence tags was selected and characterized, in terms of the ability of these sequences to form specific duplex structures with their complementary sequences, and the potential for cross-hybridization within the sequence set. See Example 4, below. The subset of 100 sequences was randomly selected, and analyzed using the Luminex¹⁰⁰ LabMAP™ platform. The 100 sequences were chemically immobilized onto the set of 100 different Luminex microsphere populations, such that each specific sequence was coupled to one spectrally distinct microsphere population. The pool of 100 microsphere-immobilized probes was then hybridized with each of the 100 corresponding complementary sequences. Each sequence was examined individually for its specific hybridization with its complementary sequence, as well as for its non-specific hybridization with the other 99 sequences present in the reaction. This analysis demonstrated the propensity of each sequence to hybridize only to its complement (perfect match), and not to cross-hybridize appreciably with any of the other oligonucleotides present in the hybridization reaction.

It is within the capability of a person skilled in the art, given the family of sequences of Table II, to modify the sequences, or add other sequences while largely retaining the property of minimal cross-hybridization which the polynucleotides of Table II have been demonstrated to have.

There are 1168 polynucleotide sequences given in Table II. Since all 1168 of this family of polynucleotides can work with each other as a minimally cross-hybridizing set, then any plurality of polynucleotides that is a subset of the 1168 can also act as a minimally cross-hybridizing set of polynucleotides. An application in which, for example, 30 molecules are to be sorted using a family of polynucleotide tags and tag complements could thus use any group of 30 sequences shown in Table II. This is not to say that some subsets may be found in a practical sense to be more preferred than others. For example, it may be found that a particular subset is more tolerant of a wider variety of conditions under which hybridization is conducted before the degree of cross-hybridization becomes unacceptable.

It may be desirable to use polynucleotides that are shorter in length than the 24 bases of those in Table II. A family of subsequences (i.e., subframes of the sequences illustrated) based on those contained in Table II having as few as 10 bases per sequence could be chosen, so long as the subsequences are chosen to retain homological properties between any two of the sequences of the family important to their non cross-hybridization.

The selection of sequences using this approach would be amenable to a computerized process. Thus for example, a string of 10 contiguous bases of the first 24mer of Table II could be selected: AAATTGTGAAAGATTGTTTGTGTA (SEQ ID NO:1).

The same string of contiguous bases from the second 24mer could then be selected and compared for similarity against the first chosen sequence: GTTAGAGTTAATTGTATTTGATGA (SEQ ID NO:2 of Table II). A systematic pairwise comparison could then be carried out to determine if the similarity requirements are violated. If the pair of sequences does not violate any set property, a 10mer subsequence can be selected from the third 24mer sequence of Table II, and compared to each of the first two 10mer sequences (in a pairwise fashion to determine its compatibility therewith, etc. In this way a family of 10mer sequences may be developed.

It is within the scope of this invention, to obtain families of sequences containing 1mer, 12mer, 13mer, 14mer, 15mer, 16mer, 17mer, 18mer, 19mer, 20mer, 21mer, 22mer and 23mer sequences by analogy to that shown for 10mer sequences. It may be desirable to have a family of sequences in which there are sequences greater in length than the 24mer sequences shown in Table II. It is within the capability of a person skilled in the art, given the family of sequences shown in Table II, to obtain such a family of sequences. One possible approach would be to insert into each sequence at one or more locations a nucleotide, non-natural base or analogue such that the longer sequence should not have greater similarity than any two of the original non-cross-hybridizing sequences of Table II and the addition of extra bases to the tag sequences should not result in a major change in the thermodynamic properties of the tag sequences of that set for example the GC content must be maintained between 10%-40% with a variance from the average of 20%. This method of inserting bases could be used to obtain, for example, a family of sequences up to 40 bases long.

Given a particular family of sequences that can be used as a family of tags (or tag complements), e.g., those of Table II, a skilled person will readily recognize variant families that work equally as well.

Again taking the sequences of Table II for example, every T could be converted to an A and vice versa and no significant change in the cross-hybridization properties would be expected to be observed. This would also be true if every G were converted to a C.

Also, all of the sequences of a family could be taken to be constructed in the 5′-3′ direction, as is the convention, or all of the constructions of sequences could be in the opposition direction (3′-5′).

There are additional modifications that can be carried out. For example, C has not been used in the family of sequences. Substitution of C in place of one or more G's of a particular sequence would yield a sequence that is at least as low in homology with every other sequence of the family as was the particular sequence chosen for modification. It is thus possible to substitute C in place of one or more G's in any of the sequences shown in Table II. Analogously, substituting of C in place of one or more A's is possible, or substituting C in place of one or T's is possible.

It is preferred that the sequences of a given family are of the same, or roughly the same length. Preferably, all the sequences of a family of sequences of this invention have a length that is within five bases of the base-length of the average of the family. More preferably, all sequences are within four bases of the average base-length. Even more preferably, all or almost all sequences are within three bases of the average base-length of the family. Better still, all or almost all sequences have a length that is within two of the base-length of the average of the family, and even better still, within one of the base-length of the average of the family.

It is also possible for a person skilled in the art to derive sets of sequences from the family of sequences described in this specification and remove sequences that would be expected to have undesirable hybridization properties.

### Example 3

### Cross Talk Behavior of Sequence on Beads

A group of 100 of the sequences of Table I was tested for feasibility for use as a family of minimally cross-hybridizing oligonucleotides. The 100 sequences selected are separately indicated in Table I along with the numbers assigned to the sequences in the tests.

The tests were conducted using the Luminex LabMAP™ platform available from Luminex Corporation, Austin, Tex., U.S.A. The one hundred sequences used as probes; were synthesized as oligonucleotides by Integrated DNA Technologies (IDT, Coralville, Iowa, U.S.A.). Each probe included a C₆ aminolink group coupled to the 5′-end of the oligonucleotide through a C₁₂ ethylene glycol spacer. The C₆ aminolink molecule is a six carbon spacer containing an amine group that can be used for attaching the oligonucleotide to a solid support. One hundred oligonucleotide targets (probe complements), the sequence of each being the reverse complement of the 100 probe sequences, were also synthesized by IDT. Each target was labelled at its 5′-end with biotin. All oligonucleotides were purified using standard desalting procedures, and were reconstituted to a concentration of approximately 200 μM in sterile, distilled water for use. Oligonucleotide concentrations were determined spectrophotometrically using extinction coefficients provided by the supplier.

Each probe was coupled by its amino linking group to a carboxylated fluorescent microsphere of the LabMAP system according to the Luminex¹⁰⁰ protocol. The microsphere, or bead, for each probe sequence has unique, or spectrally distinct, light absorption characteristics which permits each probe to be distinguished from the other probes. Stock bead pellets were dispersed by sonication and then vortexing. For each bead population, approximately five million microspheres (400 μL) were removed from the stock tube using barrier tips and added to a 1.5 mL Eppendorf tube (USA Scientific). The microspheres were then centrifuged, the supernatant was removed, and beads were resuspended in 25 μL of 0.2 M MES (2-(N-morpholino)ethane sulfonic acid) (Sigma), pH 4.5, followed by vortexing and sonication. One nmol of each probe (in a 25 μL volume) was added to its corresponding bead population. A volume of 2.5 μL of EDC cross-linker (1-ethyl-3-(3-dimethylaminopropyl) carbodiimide hydrochloride (Pierce), prepared immediately before use by adding 1.0 mL of sterile ddH₂O to 10 mg of EDC powder, was added to each microsphere population. Bead mixes were then incubated for 30 minutes at room temperature in the dark with periodic vortexing. A second 2.5 μL aliquot of freshly prepared EDC solution was then added followed by an additional 30 minute incubation in the dark. Following the second EDC incubation, 1.0 mL of 0.02% Tween-20 (BioShop) was added to each bead mix and vortexed. The microspheres were centrifuged, the supernatant was removed, and the beads were resuspended in 1.0 mL of 0.1% sodium dodecyl sulfate (Sigma). The beads were centrifuged again and the supernatant removed. The coupled beads were resuspended in 100 μL of 0.1 M MES pH 4.5. Bead concentrations were then determined by diluting each preparation 100-fold in ddH₂O and enumerating using a Neubauer BrightLine Hemacytometer. Coupled beads were stored as individual populations at 2-8° C. protected from light.

The relative oligonucleotide probe density on each bead population was assessed by Terminal Deoxynucleotidyl Transferase (TdT) end-labelling with biotin-ddUTPs. TdT was used to label the 3′-ends of single-stranded DNA with a labeled ddNTP. Briefly, 180 μL of the pool of 100 bead populations (equivalent to about 4000 of each bead type) to be used for hybridizations was pipetted into an Eppendorf tube and centrifuged. The supernatant was removed, and the beads were washed in 1×TdT buffer. The beads were then incubated with a labelling reaction mixture, which consisted of 5×TdT buffer, 25 mM CoCl₂, and 1000 pmol of biotin-16-ddUTP (all reagents were purchased from Roche). The total reaction volume was brought up to 85.5 μL with sterile, distilled H₂O, and the samples were incubated in the dark for 1 hour at 37° C. A second aliquot of enzyme was added, followed by a second 1 hour incubation. Samples were run in duplicate, as was the negative control, which contained all components except the TdT. In order to remove unincorporated biotin-ddUTP, the beads were washed 3 times with 200 μL of hybridization buffer, and the beads were resuspended in 50 μL of hybridization buffer following the final wash. The biotin label was detected spectrophotometrically using SA-PE (streptavidin-phycoerythrin conjugate). The streptavidin binds to biotin and the phycoerythrin is spectrally distinct from the probe beads. The 10 mg/mL stock of SA-PE was diluted 100-fold in hybridization buffer, and 15 μL of the diluted SA-PE was added directly to each reaction and incubated for 15 minutes at 37° Celsius. The reactions were analyzed on the Luminex¹⁰⁰ LabMAP. Acquisition parameters were set to measure 100 events per bead using a sample volume of 50 μL.

The results obtained are shown in FIG. 2. As can be seen the Mean Fluorescent Intensity (MFI) of the beads varies from 277.75 to 2291.08, a range of 8.25-fold. Assuming that the labelling reactions are complete for all of the oligonucleotides, this illustrates the signal intensity that would be obtained for each type of bead at this concentration if the target (i.e., labelled complement) was bound to the probe sequence to the full extent possible.

The cross-hybridization of targets to probes was evaluated as follows. 100 oligonucleotide probes linked to 100 different bead populations, as described above, were combined to generate a master bead mix, enabling multiplexed reactions to be carried out. The pool of microsphere-immobilized probes was then hybridized individually with each biotinylated target. Thus, each target was examined individually for its specific hybridization with its complementary bead-immobilized sequence, as well as for its non-specific hybridization with the other 99 bead-immobilized universal sequences present in the reaction. For each hybridization reaction, 25 μL bead mix (containing about 2500 of each bead population in hybridization buffer) was added to each well of a 96-well Thermowell PCR plate and equilibrated at 37° C. Each target was diluted to a final concentration of 0.002 fmol/μL in hybridization buffer, and 25 μL (50 fmol) was added to each well, giving a final reaction volume of 50 μL. Hybridization buffer consisted of 0.2 M NaCl, 0.1 M Tris, 0.08% Triton X-100, pH 8.0 and hybridizations were performed at 37° C. for 30 minutes. Each target was analyzed in triplicate and six background samples (i.e. no target) were included in each plate. A SA-PE conjugate was used as a reporter, as described above. The 10 mg/mL stock of SA-PE was diluted 100-fold in hybridization buffer, and 15 μL of the diluted SA-PE was added directly to each reaction, without removal of unbound target, and incubated for 15 minutes at 37° C. Finally, an additional 35 μL of hybridization buffer was added to each well, resulting in a final volume of 100 μL per well prior to analysis on the Luminex¹⁰⁰ LabMAP. Acquisition parameters were set to measure 100 events per bead using a sample volume of 80 μL.

The percent hybridization was calculated for any event in which the NET MFI was at least 3 times the zero target background. In other words, a calculation was made for any sample where (MFIₛₐₘₚₗₑ−MFI_{zero target background})/MFI_{zero target background}≧3.

A “positive” cross-talk event (i.e., significant mismatch or cross-hybridization) was defined as any event in which the net median fluorescent intensity (MFIₛₐₘₚₗₑ−MFI_{zero target background}) generated by a mismatched hybrid was greater than or equal to the arbitrarily set limit of 10% that of the perfectly matched hybrid determined under identical conditions. As there are 100 probes and 100 targets, there are 100×100=10,0000 possible different interactions possible of which 100 are the result of perfect hybridizations. The remaining 9900 result from hybridization of a target with a mismatched probe.

The results obtained are illustrated in FIG. 3. The ability of each target to be specifically recognized by its matching probe is shown of the possible 9900 non-specific hybridization events that could have occurred when the 100 targets were each exposed to the pool of 100 probes, 6 events were observed. Of these 6 events, the highest non-specific event generated a signal equivalent to 10.2% of the signal observed for the perfectly matched pair (i.e. specific hybridization event).

Each of the 100 targets was thus examined individually for specific hybridization with its complement sequence as incorporated onto a microsphere, as well as for non-specific hybridization with the complements of the other 99 target sequences. Representative hybridization results for target 16 (complement of probe -16, Table I) are shown in FIG. 4. Probe 16 was found to hybridize only to its perfectly-matched target. No cross-hybridization with any of the other 99 targets was observed.

The foregoing results demonstrate the possibility of incorporating the 210 sequences of Table I, or any subset thereof, into a multiplexed system with the expectation that most if not all sequences can be distinguished from the others by hybridization. That is, it is possible to distinguish each target from the other targets by hybridization of the target with its precise complement and minimal hybridization with complements of the other targets.

Methods for Synthesis of Oligonucleotide Families

Preferably oligonucleotide sequences of the invention are synthesized directly by standard phosphoramidite synthesis approaches and the like (Caruthers et al, Methods in Enzymology; 154, 287-313: 1987; Lipshutz et al, Nature Genet.; 21, 20-24: 1999; Fodor et al, Science; 251, 763-773: 1991). Alternative chemistries involving non natural bases such as peptide nucleic acids or modified nucleosides that offer advantages in duplex stability may also be used (Hacia et al; Nucleic Acids Res; 27: 4034-4039, 1999; Nguyen et al, Nucleic Acids Res.; 27, 1492-1498: 1999; Weiler et al, Nucleic Acids Res.; 25, 2792-2799:1997). It is also possible to synthesize the oligonucleotide sequences of this invention with alternate nucleotide backbones such as phosphorothioate or phosphoroamidate nucleotides. Methods involving synthesis through the addition of blocks of sequence in a stepwise-manner may also be employed (Lyttle et al, Biotechniques, 19: 274-280 (1995). Synthesis may be carried out directly on the substrate to be used as a solid phase support for the application or the oligonucleotide can be cleaved from the support for use in solution or coupling to a second support.

Solid Phase Supports

There are several different solid phase supports that can be used with the invention. They include but are not limited to slides, plates, chips, membranes, beads, microparticles and the like. The solid phase supports can also vary in the materials that they are composed of including plastic, glass, silicon, nylon, polystyrene, silica gel, latex and the like. The surface of the support is coated with the complementary tag sequences by any conventional means of attachment.

In preferred embodiments, the family of tag complement sequences is derivatized to allow binding to a solid support. Many methods of derivatizing a nucleic acid for binding to a solid support are known in the art (Hermanson G., Bioconjugate Techniques; Acad. Press: 1996). The sequence tag may be bound to a solid support through covalent or non-covalent bonds (Iannone et al, Cytometry; 39: 131-140, 2000; Matson et al, Anal. Biochem.; 224: 110-106, 1995; Proudnikov et al, Anal Biochem; 259: 34-41, 1998; Zammatteo et al, Analytical Biochemistry; 280:143-150, 2000). The sequence tag can be conveniently derivatized for binding to a solid support by incorporating modified nucleic acids in the terminal 5′ or 3′ locations.

A variety of moieties useful for binding to a solid support (e.g., biotin, antibodies, and the like), and methods for attaching them to nucleic acids, are known in the art. For example, an amine-modified nucleic acid base (available from, eg., Glen Research) may be attached to a solid support (for example, Covalink-NH, a polystyrene surface grafted with secondary amino groups, available from Nunc) through a bifunctional crosslinker (e.g., bis(sulfosuccinimidyl suberate), available from Pierce). Additional spacing moieties can be added to reduce steric hindrance between the capture moiety and the surface of the solid support.

Attaching Tags to Analytes for Sorting

A family of oligonucleotide tag sequences can be conjugated to a population of analytes most preferably polynucleotide sequences in several different ways including but not limited to direct chemical synthesis, chemical coupling, ligation, amplification, and the like. Sequence tags that have been synthesized with primer sequences can be used for enzymatic extension of the primer on the target for example in PCR amplification.

Detection of Single Nucleotide Polymorphisms Using Primer Extension

There are a number of areas of genetic analysis where families of non-cross-hybridizing sequences can be applied including disease diagnosis, single nucleotide polymorphism analysis, genotyping, expression analysis and the like. One such approach for genetic analysis, referred to as the primer extension method (also known as Genetic Bit Analysis (Nikiforov et al, Nucleic Acids Res.; 22, 4167-4175: 1994; Head et al Nucleic Acids Res.; 25, 5065-5071: 1997)), is an extremely accurate method for identification of the nucleotide located at a specific polymorphic site within genomic DNA. In standard primer extension reactions, a portion of genomic DNA containing a defined polymorphic site is amplified by PCR using primers that flank the polymorphic site. In order to identify which nucleotide is present at the polymorphic site, a third primer is synthesized such that the polymorphic position is located immediately 3′ to the primer. A primer extension reaction is set up containing the amplified DNA, the primer for extension, up to 4 dideoxynucleoside triphosphates (each labeled with a different fluorescent dye) and a DNA polymerase such as the Klenow subunit of DNA Polymerase 1. The use of dideoxy nucleotides ensures that a single base is added to the 3′ end of the primer, a site corresponding to the polymorphic site. In this way the identity of the nucleotide present at a specific polymorphic site can be determined by the identity of the fluorescent dye-labeled nucleotide that is incorporated in each reaction. One major drawback to this approach is its low throughput. Each primer extension reaction is carried out independently in a separate tube.

Universal sequences can be used to enhance the throughput of primer extension assay as follows. A region of genomic DNA containing multiple polymorphic sites is amplified by PCR. Alternatively, several genomic regions containing one or more polymorphic sites each are amplified together in a multiplexed PCR reaction. The primer extension reaction is carried out as described above except that the primers used are chimeric, each containing a unique universal tag at the 5′ end and the sequence for extension at the 3′ end. In this way, each gene-specific sequence would be associated with a specific universal sequence. The chimeric primers would be hybridized to the amplified DNA and primer extension is carried out as described above. This would result in a mixed pool of extended primers, each with a specific fluorescent dye characteristic of the incorporated nucleotide. Following the primer extension reaction, the mixed extension reactions are hybridized to an array containing probes that are reverse complements of the universal sequences on the primers. This would segregate the products of a number of primer extension reactions into discrete spots. The fluorescent dye present at each spot would then identify the nucleotide incorporated at each specific location. A number of additional methods for the detection of single nucleotide polymorphisms, including but not limited to, allele specific polymerase chain reaction (ASPCR), allele specific primer extension (ASPE) and oligonucleotide ligation assay (OLA) can be performed by someone skilled in the art in combination with the tag sequences described herein.

Kits Using Families Of Tag Sequences

The families of non cross-hybridizing sequences may be provided in kits for use in for example genetic analysis. Such kits include at least one set of non-cross-hybridizing sequences in solution or on a solid support. Preferably the sequences are attached to microparticles and are provided with buffers and reagents that are appropriate for the application. Reagents may include enzymes, nucleotides, fluorescent labels and the like that would be required for specific applications. Instructions for correct use of the kit for a given application will be provided.

### EXAMPLES

### Example 4

### Cross Talk Behavior of Sequence on Beads

A group of 100 sequences, randomly selected from Table II, was tested for feasibility for use as a family of minimally cross-hybridizing oligonucleotides. The 100 sequences selected are separately indicated in Table II along with the numbers assigned to the sequences in the tests.

The tests were conducted using the Luminex LabMAP™ platform available from Luminex Corporation, Austin, Tex., U.S.A. The one hundred sequences, used as probes, were synthesized as oligonucleotides by Integrated DNA Technologies (IDT, Coralville, Iowa, U.S.A.). Each probe included a C₆ aminolink group coupled to the 5′-end of the oligonucleotide through a C₁₂ ethylene glycol spacer. The C₆ aminolink molecule is a six carbon spacer containing an amine group that can be used for attaching the oligonucleotide to a solid support. One hundred oligonucleotide targets (probe complements), the sequence of each being the reverse complement of the 100 probe sequences, were also synthesized by IDT. Each target was labelled at its 5′-end with biotin. All oligonucleotides were purified using standard desalting procedures, and were reconstituted to a concentration of approximately 200 μM in sterile, distilled water for use. Oligonucleotide concentrations were determined spectrophotometrically using extinction coefficients provided by the supplier.

Each probe was coupled by its amino linking group to a carboxylated fluorescent microsphere of the LapMAP system according to the Luminex¹⁰⁰ protocol. The microsphere, or bead, for each probe sequence has unique, or spectrally distinct, light absorption characteristics which permits each probe to be distinguished from the other probes. Stock bead pellets were dispersed by sonication and then vortexing. For each bead population, five million microspheres (400 μL) were removed from the stock tube using barrier tips and added to a 1.5 mL Eppendorf tube (USA Scientific). The microspheres were then centrifuged, the supernatant was removed, and beads were resuspended in 25 μL of 0.2 M MES (2-(N-morpholino)ethane sulfonic acid) (Sigma), pH 4.5, followed by vortexing and sonication. One nmol of each probe (in a 25 μL volume) was added to its corresponding bead population. A volume of 2.5 μL of EDC cross-linker (1-ethyl-3-(3-dimethylaminopropyl) carbodiimide hydrochloride (Pierce), prepared immediately before use by adding 1.0 mL of sterile ddH₂₀ to 10 mg of EDC powder, was added to each microsphere population. Bead mixes were then incubated for 30 minutes at room temperature in the dark with periodic vortexing. A second 2.5 μL aliquot of freshly prepared. EDC solution was then added followed by an additional 30 minute incubation in the dark. Following the second EDC incubation, 1.0 mL of 0.02% Tween-20 (BioShop) was added to each bead mix and vortexed. The microspheres were centrifuged, the supernatant was removed, and the beads were resuspended in 1.0 mL of 0.1% sodium dodecyl sulfate (Sigma). The beads were centrifuged again and the supernatant removed. The coupled beads were resuspended in 100 μL of 0.1 M MES pH 4.5. Bead concentrations were then determined by diluting each preparation 100-fold in ddH₂O and enumerating using a Neubauer BrightLine Hemacytometer. Coupled beads were stored as individual populations at 8° C. protected from light.

The relative oligonucleotide probe density on each bead population was assessed by Terminal Deoxynucleotidyl Transferase (TdT) end-labelling with biotin-ddUTPs. TdT was used to label the 3′-ends of single-stranded DNA with a labeled ddNTP. Briefly, 180 μL of the pool of 100 bead populations (equivalent to about 4000 of each bead type) to be used for hybridizations was pipetted into an Eppendorf tube and centrifuged. The supernatant was removed, and the beads were washed in 1×TdT buffer. The beads were then incubated with a labelling reaction mixture, which consisted of 5×TdT buffer, 25 mM CoCl₂, and 1000 pmol of biotin-16-ddUTP (all reagents were purchased from Roche). The total reaction volume was brought up to 85.5 μL with sterile, distilled H₂O, and the samples were incubated in the dark for 1 hour at 37° C. A second aliquot of enzyme was added, followed by a second 1 hour incubation. Samples were run in duplicate, as was the negative control, which contained all components except the TdT. In order to remove unincorporated biotin-ddUTP, the beads were washed 3 times with 200 μL of hybridization buffer, and the beads were resuspended in 50 μL of hybridization buffer following the final wash. The biotin label was detected spectrophotometrically using SA-PE (streptavidin-phycoerythrin conjugate). The streptavidin binds to biotin and the phycoerythrin is spectrally distinct from the probe beads. The 10 mg/mL stock of SA-PE was diluted 100-fold in hybridization buffer, and 15 μL of the diluted SA-PE was added directly to each reaction and incubated for 15 minutes at 37° Celsius. The reactions were analyzed on the Luminex¹⁰⁰ LabMAP. Acquisition parameters were set to measure 100 events per bead using a sample volume of 50 μL.

The results obtained are shown in FIG. 6. As can be seen the Mean Fluorescent Intensity (MFI) of the beads varies from 840.3 to 3834.9, a range of 4.56-fold. Assuming that the labelling reactions are complete for all of the oligonucleotides, this illustrates the signal intensity that would be obtained for each type of bead at this concentration if the target (i.e., labelled complement) was bound to the probe sequence to the full extent possible.

The cross-hybridization of targets to probes was evaluated as follows. 100 oligonucleotide probes linked to 100 different bead populations, as described above, were combined to generate a master bead mix, enabling multiplexed reactions to be carried out. The pool of microsphere-immobilized probes was then hybridized individually with each biotinylated target. Thus, each target was examined individually for its specific hybridization with its complementary bead-immobilized sequence, as well as for its non-specific hybridization with the other 99 bead-immobilized universal sequences present in the reaction. For each hybridization reaction, 25 μL bead mix (containing about 2500 of each bead population in hybridization buffer) was added to each well of a 96-well Thermowell PCR plate and equilibrated at 37° C. Each target was diluted to a final concentration of 0.002 fmol/LL in hybridization buffer, and 25 μL (50 fmol) was added to each well, giving a final reaction volume of 50 μL. Hybridization buffer consisted of 0.2 M NaCl, 0.1 M Tris, 0.08% Triton X-100, pH 8.0 and hybridizations were performed at 37° C. for 30 minutes. Each target was analyzed in triplicate and six background samples (i.e. no target) were included in each plate. A SA-PE conjugate was used as a reporter, as described above. The 10 mg/mL stock of SA-PE was diluted 100-fold in hybridization buffer, and 15 μL of the diluted SA-PE was added directly to each reaction, without removal of unbound target, and incubated for 15 minutes at 37° C. Finally, an additional 35 μL of hybridization buffer was added to each well, resulting in a final volume of 100 μL per well prior to analysis on the Luminex¹⁰⁰ LabMAP. Acquisition parameters were set to measure 100 events per bead using a sample volume of 80 μL.

The percent hybridization was calculated for any event in which the NET MFI was at least 3 times the zero target background. In other words, a calculation was made for any sample where (MFIₛₐₘₚₗₑ−MFI_{zero target background})/MFI_{zero target background}≧3.

The net median fluorescent intensity (MFIₛₐₘₚₗₑ−MFI_{zero target background}) generated for all of the 10,000 possible target/probe combinations was calculated. As there are 100 probes and 100 targets, there are 100×100=10,0000 possible different interactions possible of which 100 are the result of perfect hybridizations. The remaining 9900 result from hybridization of a target with a mismatched probe. A cross-hybridization event is then defined as a non-specific event whose net median fluorescent intensity exceeds 3 times the zero target background. In other words, a cross-talk calculation is only be made for any sample where (MFIₛₐₘₚₗₑ−MFI_{zero target background})/MFI_{zero target background}≧3. Cross-hybridization events were quantified by expressing the value of the cross-hybridization signal as a percentage of the perfect match hybridization signal with the same probe.

The results obtained are illustrated in FIG. 7. The ability of each target to be specifically recognized by its matching probe is shown of the possible 9900 non-specific hybridization events that could have occurred when the 100 targets were each exposed to the pool of 100 probes, 6 events were observed. Of these 6 events, the highest non-specific event generated a signal equivalent to 5.3% of the signal observed for the perfectly matched pair (i.e. specific hybridization event).

Each of the 100 targets was thus examined individually for specific hybridization with its complement sequence as incorporated onto a microsphere, as well as for non-specific hybridization with the complements of the other 99 target sequences. Representative hybridization results for target (complement of probe 90, Table II) are shown in FIG. 8. Probe 90 was found to hybridize only to its perfectly-matched target. No cross-hybridization with any of the other 99 targets was observed.

The foregoing results demonstrate the possibility of incorporating the 1168 sequences of Table II, or any subset thereof, into a multiplexed system with the expectation that most if not all sequences can be distinguished from the others by hybridization. That is, it is possible to distinguish each target from the other targets by hybridization of the target with its precise complement and minimal hybridization with complements of the other targets.

### Example 5

### Tag Sequences Used in Sorting Polynucleotides

The family of non cross hybridizing sequence tags or a subset thereof can be attached to oligonucleotide probe sequences during synthesis and used to generate amplified probe sequences. In order to test the feasibility of PCR amplification with non cross hybridizing sequence tags and subsequently addressing each respective sequence to its appropriate location on two-dimensional or bead arrays, the following experiment was devised. A 24mer tag sequence can be connected in a 5′-3′ specific manner to a p53 exon specific sequence (20mer reverse primer). The connecting p53 sequence represents the inverse complement of the nucleotide gene sequence. To facilitate the subsequent generation of single stranded DNA post-amplification the tag-Reverse primer can be synthesized with a phosphate modification (PO₄) on the 5′-end. A second PCR primer can also be generated for each desired exon, represented by the Forward (5′-3′) amplification primer. In this instance the Forward primer can be labeled with a 5′-biotin modification to allow detection with Cy3-avidin or equivalent.

A practical example of the aforementioned description is as follows: For exon 1 of the human p53 tumor suppressor gene sequence the following tag-Reverse primer (SEQ ID NO: 1171) can be generated:

```
                                                    222087 222063
5′-P04- [symbol] -TCCAGGGAAGCGTGTCACCGTCGT-3′
            Tag Sequence # 3 Exon 1 Reverse
```

The numbering above the Exon-1 reverse primer represents the genomic nucleotide positions of the indicated bases. The corresponding Exon-1 Forward primer sequence (SEQ ID NO:1172) is as follows:

221873 221896
5′-Biotin-TCATGGCGACTGTCCAGCTTTGTG-3′

In combination, these primers will amplify a product of 214 bp plus a 24 bp tag extension yielding a total size of 238 bp. Once amplified, the PCR product can be purified using a QIAquick PCR purification kit and the resulting DNA can be quantified. To generate single stranded DNA, the DNA is subjected to λ-exonuclease digestion thereby resulting in the exposure of a single stranded sequence (anti-tag) complementary to the tag-sequence covalently attached to the solid phase array. The resulting product is heated to 95° C. for 5 minutes and then directly applied to the array at a concentration of 10-50 nM. Following hybridization and concurrent sorting, the tag-Exon 1 sequences are visualized using Cy3-streptavidin. In addition to direct visualization of the biotinylated product, the product itself can now act as a substrate for further analysis of the amplified region, such as SNP detection and haplotype determination.

The Invader Assay is described in detail in U.S. Pat. Nos. 5,846,717 and 5,985,557. Briefly, the ability of the Invader technology to identify target nucleic acid sequences and in particular single base pair changes is dependent on the proper structure being formed, followed by subsequent recognition and cleavage of this structure by the Cleavase enzyme. For recognition by Cleavase III, the target sequence must be complementary to the primary probe, and there must be at least a 1 base “invasion” (overlap) of this structure by an upstream oligonucleotide. Cleavable “flaps’ can be created by invasion of an upstream oligonucleotide without primer extension, and the site of cleavage is determined by the extent to which the upstream oligonucleotide overlaps the 5′ region of the downstream oligonucleotide. Cleavage by the Cleavase enzyme is dependent on this invaded structure and is sensitive to single-base mismatches is positioned immediately upstream of the cleavage site. By adding overlapping pairs of oligonucleotide probes complementary to a predetermined region of target DNA, the cleavage of the downstream probes become a sensitive indicator of the presence of the target sequence. Further, reaction conditions have been established that allow multiple copies of the downstream oligonucleotide probe to be cleaved for each target sequence without temperature cycling, so as to amplify the cleavage signal and allow quantitative detection of target DNA at sub-attomole levels. Incorporation of the minimally cross-hybridizing sequences of the invention described herein into the probe that will be cleaved by the Cleavase enzyme allows detection of multiple target DNA sequences in a single experiment.

Definitions

Non-cross-hybridization: Describes the absence of hybridization between two sequences that are not perfect complements of each other.

Cross-hybridization: The hydrogen bonding of a single-stranded DNA sequence that is partially but not entirely complementary to a single-stranded substrate.

Homology or Similarity: How closely related two or more separate strands of DNA are to each other, based on their base sequences.

Analogue: The symbols A, G, T/U, C take on their usual meaning in the art here. In the case of T and U, a person skilled in the art would understand that these are equivalent to each other with respect to the inter-strand hydrogen-bond (Watson-Crick) binding properties at work in the context of this invention. The two bases are thus interchangeable and hence the designation of T/U. A chemical, which resembles a nucleotide base is an analogue thereof. A base that does not normally appear in DNA but can substitute for the ones, which do, despite minor differences in structure. Analogues particularly useful in this invention are of the naturally occurring bases can be inserted in their respective places where desired. Such an analogue is any non-natural base, such as peptide nucleic acids and the like that undergoes normal Watson-Crick pairing in the same way as the naturally occurring nucleotide base to which it corresponds.

Complement: The opposite or “mirror” image of a DNA sequence. A complementary DNA sequence has an “A” for every “T” and a “C” for every “G”. Two complementary strands of single stranded DNA, for example a tag sequence and its complement, will join to form a double-stranded molecule.

Complementary DNA (cDNA): DNA that is synthesized from a messenger RNA template; the single-stranded form is often used as a probe in physical mapping.

Oligonucleotide: Refers to a short nucleotide polymer whereby the nucleotides may be natural nucleotide bases or analogues thereof.

Tag: Refers to an oligonucleotide that can be used for specifically sorting analytes with at least one other oligonucleotide that when used together do not cross hybridize.

Similar Homology: In the context of this invention, pairs of sequences are compared with each other based on the amount of “homology” between the sequences. By way of example, two sequences are said to have a 50% “maximum homology” with each other if, when the two sequences are aligned side-by-side with each other so to obtain the (absolute) maximum number of identically paired bases, the number of identically paired bases is 50% of the total number of bases in one of the sequences. (If the sequences being compared are of different lengths, then it would be of the total number of bases in the shorter of the two sequences.) Examples of determining maximum homology are as follows:

### Example 1

| | |
|---|---|
| * * | |
| A-A-B-B-C-C | |
| B-D-C-D-D-D | (2 out of 4 paired bases are the |
| | same) |
| * * | |
| A-A-B-B-C-C | |
| B-D-C-D-D-D | (2 out of 3 paired bases are the |
| | same) |

In this case, the maximum number of identically paired bases is two and there are two possible alignments yielding this maximum number. The total number of possible pairings is six giving 33⅓% ( 2/6) homology. The maximum amount of homology between the two sequences is thus ⅓.

### Example 2

| | |
|---|---|
| * * * | |
| A-A-B-B-C-A | |
| A-A-D-D-C-D | (3 out of 6 paired bases are the same) |

In this alignment, the number of identically paired bases is three and the total number of possibly paired bases is six, so the homology between the two sequences is 3/6(50%).

| | |
|---|---|
| * | |
| A-A-B-B-C-A | |
| A-A-D-D-C-D | (1 out of 1 paired bases are |
| | the same) |

In this alignment, the number of identically paired bases is 1, so the homology between the two sequences is ⅙ (16⅔%)

The maximum homology between these two sequences is thus 50%.

Block sequence: Refers to a symbolic representation of a sequence of blocks. In its most general form a block sequence is a representative sequence in which no particular value., mathematical variable, or other designation is assigned to each block of the sequence.

Incidence Matrix: As used herein is a well-defined term in the field of Discrete Mathematics. However, an incidence matrix cannot be defined without first defining a “graph”. In the method described herein a subset of general graphs called simple graphs is used. Members of this subcategory are further defined as follows.

A simple graph G is a pair (V, E) where V represents the set of vertices of the simple graph and E is a set of un-oriented edges of the simple graph. An edge is defined as a 2-component combination of members of the set of vertices. In other words, in a simple graph G there are some pairs of vertices that are connected by an edge. In our application a graph is based on nucleic acid sequences generated using sequence templates and vertices represent DNA sequences and edges represent a relative property of any pair of sequences.

The incidence matrix is a mathematical object that allows one to describe any given graph. For the subset of simple graphs used herein, the simple graph G=(V,E), and for a pre-selected and fixed ordering of vertices, V={v₁,v₂, . . . vₙ}, elements of the incidence matrix A(G)=[aᵢⱼ] are defined by the following rules:
(1) aᵢⱼ=1 for any pair of vertices {vᵢ,vⱼ} that is a member of the set of edges; and
(2) aᵢⱼ=0 for any pair of vertices {vᵢ,vⱼ}that is not a member of the set of edges.
   This is an exact unequivocal definition of the incidence matrix. In effect, one selects the indices: 1, 2, . . . n of the vertices and then forms an (n×n) square matrix with elements aᵢⱼ=1 if the vertices vᵢ and vⱼ are connected by an edge and aᵢⱼ=0 if the vertices vᵢ and vⱼ are not connected by an edge.

To define the term “class property” as used herein, the term “complete simple graph” or “clique” must first be defined. The complete simple graph is required because all sequences that result from the method described herein should collectively share the relative property of any pair of sequences defining an edge of graph G, for example not violating the threshold rule that is, do not have a “maximum simple homology” greater than a predetermined amount, whatever pair of the sequences are chosen from the final set. It is possible that additional “local” rules, based on known or empirically determined behavior of particular nucleotides, or nucleotide sequences, are applied to sequence pairs in addition to the basic threshold rule.

In the language of a simple graph, G=(V, E), this means in the final graph there should be no pair of vertices (no sequence pair) not connected by an edge (because an edge means that the sequences represented by vᵢ and vⱼ do not violate the threshold rule).

Because the incidence matrix of any simple graph can be generated by the above definition of its elements, the consequence of defining a simple complete graph is that the corresponding incidence matrix for a simple complete graph will have all off-diagonal elements equal to 1 and all diagonal elements equal to 0. This is because if one aligns a sequence with itself, the threshold rule is of course violated, and all other sequences are connected by an edge.

For any simple graph, there might be a complete subgraph. First, the definition of a subgraph of a graph is as follows. The subgraph Gs=(Vs, Es) of a simple graph G=(V, E) is a simple graph that contains the subsets of vertices Vs of the set V of vertices and inclusion of the set Vs into the set V is immersion (a mathematical term). This means that one generates a subgraph Gs=(Vs,Es) of a simple graph G in two steps. First select some vertices Vs from G. Then select those edges Es from G that connect the chosen vertices and do not select edges that connect selected with non selected vertices.

We desire a subgraph of G that is a complete simple graph. By using this property of the complete simple graph generated from the simple graph G of all sequences generated by the template based algorithm, the pairwise property of any pair of the sequences (violating/non-violating the threshold rule) is converted into the property of all members of the set, termed “the class property”.

By selecting a subgraph of a simple graph G that is a complete simple graph, this assures that, up to the tests involving the local rules described herein, there are no pairs of sequences in the resulting set that violate the threshold rule, also described above, independent of which pair of sequences in the set are chosen. This feature is called the “desired class property”.

The present invention thus includes reducing the potential for non cross-hybridization behavior by taking into account local homologies of the sequences and appears to have greater rigor than known approaches. For example, the method described herein involves the sliding of one sequence relative to the other sequence in order to form a sequence alignment that would accommodate insertions or deletions. (Kane et al., Nucleic Acids Res.; 28, 4552-4557: 2000).

TABLE I

| | | |
|---|---|---|
| | | No. Assigned |
| SEQ ID NO (1) | Sequence | in Example 3 |

| | | |
|---|---|---|
| 1 | GATTTGTATTGATTGAGATTAAAG | 1 |
| 2 | TGATTGTAGTATGTATTGATAAAG | 2 |
| 3 | GATTGTAAGATTTGATAAAGTGTA | 3 |
| 4 | GATTTGAAGATTATTGGTAATGTA | 4 |
| 5 | GATTGATTATTGTGATTTGAATTG | 5 |
| 6 | GATTTGATTGTAAAAGATTGTTGA | 6 |
| 7 | ATTGGTAAATTGGTAAATGAATTG | 7 |
| 8 | ATTGGATTTGATAAAGGTAAATGA | |
| 9 | GTAAGTAATGAATGTAAAAGGATT | 8 |
| 10 | GATTGATTGATTGATTGATTTGAT | |
| 11 | TGATGATTAAAGAAAGTGATTGAT | |
| 12 | AAAGGATTTGATTGATAAAGTGAT | |
| 13 | TGTAGATTTGTATGTATGTATGAT | 10 |
| 14 | GATTTGATAAAGAAAGGATTGATT | |
| 15 | GATTAAAGTGATTGATGATTTGTA | 11 |
| 16 | AAAGAAAGAAAGAAAGAAAGTGTA | 12 |
| 17 | TGTAAAAGGATTGATTTGTATGTA | |
| 18 | AAAGTGTAGATTGATTAAAGAAAG | |
| 19 | AAAGTTGATTGATTGAAAAGGTAT | |
| 20 | TTGATTGAGATTGATTTTGAGTAT | |
| 21 | TGAATTGATGAATGAATGAAGTAT | 15 |
| 22 | GTAATGAAGTATGTATGTAAGTAA | |
| 23 | TGATGATTTGAATGAAGATTGATT | 16 |
| 24 | TGATAAAGTGATAAAGGATTAAAG | 17 |
| 25 | TGATTTGAGTATTTGAGATTTTGA | 18 |
| 26 | TGTAGTAAGATTGATTAAAGGTAA | |
| 27 | GTATAAAGGATTGATTTTGAAAAG | |
| 28 | GTATTTGAGTAAGTAATTGATTGA | 19 |
| 29 | GTAAAAAGTTGAGTATTGAAAAAG | |
| 30 | GATTTGATAAAGGATTTGTATTGA | |
| 31 | GATTGTATTGAAGTATTGTAAAAG | 20 |
| 32 | TGATGATTTTGATGAAAAAGTTGA | |
| 33 | TGATTTGAGATTAAAGAAAGGATT | 21 |
| 34 | TGATTGAATTGAGTAAAAAGGATT | 22 |
| 35 | AAAGTGTAAAAGGATTTGATGTAT | |
| 36 | AAAGGTATTTGAGATTTGATTGAA | |
| 37 | AAAGTTGAGATTTGAATGATTGAA | 23 |
| 38 | TGTATTGAAAAGGTATGATTTGAA | |
| 39 | GTATTGTATTGAAAAGGTAATTGA | 24 |
| 40 | TTGAGTAATGATAAAGTGAAGATT | |
| 41 | TGAAGATTTGAAGTAATTGAAAAG | 25 |
| 42 | TGAAAAAGTGTAGATTTTGAGTAA | 26 |
| 43 | TGTATGAATGAAGATTTGATTGTA | |
| 44 | AAAGTTGAGTATTGATTTGAAAAG | 27 |
| 45 | GATTTGTAGATTTGTATTGAGATT | |
| 46 | AAAGAAAGGATTTGTAGTAAGATT | 29 |
| 47 | GTAAAAAGAAAGGTATAAAGGTAA | 30 |
| 48 | GATTAAAGTTGATTGAAAAGTGAA | 31 |
| 49 | TGAAAAAGGTAATTGATGTATGAA | |
| 50 | AAAGGATTAAAGTGAAGTAATTGA | 33 |
| 51 | ATGAATTGGTATGTATATGAATGA | 34 |
| 52 | TGAAATGAATGAATGATGAAATTG | 35 |
| 53 | ATTGATTGTGAATGAAATGAATTG | 36 |
| 54 | ATTGAAAGATGAAAAGATGAAAAG | 37 |
| 55 | ATTGTTGAAAAGTGTAATGATTGA | 38 |
| 56 | ATGATGTAATGAAAAGATTGTGTA | 39 |
| 57 | AAAGATTGAAAGATGATGTAATTG | |
| 58 | ATTGATGAGTATATTGTGTAGTAA | 41 |
| 59 | AAAGATTGTGTAATTGATGATGAA | |
| 60 | AAAGGTATATTGTGTAATGAGTAA | |
| 61 | TGTAATGAGTATTGTAATTGAAAG | 43 |
| 62 | GTATAAAGAAAGATTGGTAAATGA | 44 |
| 63 | TTGAGTAATTGAATTGTGAAATGA | 45 |
| 64 | TGTATTGAATGAATTGTTGATGTA | 46 |
| 65 | TGTAATTGGTAAATGAGTAAAAAG | |
| 66 | TGAATGAAATTGATGAGTATAAAG | |
| 67 | GTAAGTAAATTGAAAGATTGATGA | 49 |
| 68 | GTAAATGATGATATTGGTATATTG | 50 |
| 69 | ATTGTTGATGATTGATTGAAATGA | 51 |
| 70 | ATTGTGAAGTATAAAGATGATTGA | 52 |
| 71 | ATGAAAAGTTGAGTAAATTGTGAT | |
| 72 | ATGAATTGAAAGTGATTGAAAAAG | 54 |
| 73 | GTAAATTGATGAAAAGTTGATGAT | |
| 74 | AAAGTGATGTATATGAGTAAATTG | 56 |
| 75 | GTAATGATAAAGATGATGATATTG | 57 |
| 76 | TTGAAAAGATTGGTAATGATATGA | |
| 77 | AAAGTGAAAAAGATTGATTGATGA | 59 |
| 78 | ATTGATGAGATTGATTATTGTGTA | |
| 79 | ATGAGATTATTGGATTTGTAGATT | 60 |
| 80 | TGAAGATTATGAATTGGTAAGATT | 61 |
| 81 | ATTGGATTATGAGATTATGATTGA | 62 |
| 82 | ATTGTTGAATTGGATTAAAGATGA | |
| 83 | AAAGATGAGTAAGTAAATTGGATT | |
| 84 | AAAGGTAAGATTATTGATGAAAAG | 65 |
| 85 | ATTGATGAGATTAAAGTTGAATTG | |
| 86 | GATTATTGGATTATGAAAAGGATT | |
| 87 | GATTTGTAATTGTTGAGTAAATGA | 67 |
| 88 | AAAGAAAGATTGTTGAGATTATGA | 68 |
| 89 | GTATAAAGGATTTTGAATTGATGA | |
| 90 | TTGAGATTGTAAATGAATTGTTGA | |
| 91 | GTATATTGATTGTGTAATGAAAAG | |
| 92 | TGATATGAATTGGATTATTGGTAT | 70 |
| 93 | ATGAATGATGAATGATGATTATTG | |
| 94 | ATGAATTGATTGGATTGTAATGAT | 71 |
| 95 | GATTGTAATTGAGTAAATTGATGA | |
| 96 | GATTATTGGATTAAAGGTAAATGA | 72 |
| 97 | ATTGTTGAATTGATGAGATTTGAT | 73 |
| 98 | GATTATGAGTAAATTGATTGTGAT | |
| 99 | GATTATTGTTGATGAATGATATTG | |
| 100 | TGTAAAAGATTGAAAGGTATGATT | 75 |
| 101 | GTATTTAGATGAGTTTGTTAGATT | 76 |
| 102 | TGAAGTTATGTAATAGAAAGTGAT | |
| 103 | GTATGTATTGTATGTAGTTAATTG | 77 |
| 104 | TGATATAGATAGTTAGATAGATAG | 78 |
| 105 | ATGATGATGTATTGTAGTTATGAA | 79 |
| 106 | TTAGTGAATGTATTAGTTGATGTA | |
| 107 | GTTAGTTAGATTATTGTTAGTTAG | 80 |
| 108 | GTTAATTGTGTAGTTTGTTATTGA | |
| 109 | GTTATGAAATAGTGATATTGTTAG | |
| 110 | ATTGTTAGAAAGTGTAGATTAAAG | 81 |
| 111 | ATGAGTATGTTATTAGTGTATGTA | 82 |
| 112 | TGTAATAGTGAAGTTAGATTGTAT | 83 |
| 113 | ATTGATAGATGATTAGTTAGTTGA | 84 |
| 114 | ATGAGTTTGTTTATGAGATTAAAG | |
| 115 | TGATGTTTGATTATGATGTAGTAT | 85 |
| 116 | ATGAGTTAGTTATGAATTAGATGA | |
| 117 | ATTGTTAGTGATGTTAGTAATTAG | 86 |
| 118 | TGATGTAAGTATTGATGTTAGTTT | 87 |
| 119 | GATTGTAAATAGAAAGTGAAGTAA | 88 |
| 120 | ATTGTGTATGAAGTATTGTATGAT | |
| 121 | ATAGTGATGTTATGAAGATTGTTA | |
| 122 | TTAGATGAATTGTGAAGTATTTAG | 90 |
| 123 | GTAAGTTATGATTGATGTTATGAA | 91 |
| 124 | GTATTGATGTTTAAAGTGTAATAG | 92 |
| 125 | GATTGTAAGTAAGATTGTATATTG | |
| 126 | GTTTGTATTTAGATGAATAGAAAG | 93 |
| 127 | GTTTGATTTGTAATAGTGATTGTA | |
| 128 | TGTATGTAGTATTTAGAAAGATGA | |
| 129 | ATGAATTGTGATAAAGAAAGTTAG | |
| 130 | TTAGTGTAGTAAGTTTAAAGTGTA | 95 |
| 131 | GTATGATTGTTTGTAATTAGTGAT | |
| 132 | GTTTAAAGTTAGTTGAGTTAGTAT | 96 |
| 133 | ATAGTGTATGTAGATTATGAGATT | 97 |
| 134 | TTGAATGATTAGTTGAGTATGATT | 98 |
| 135 | GTATGTAAGTTAGTATGATTTGAA | |
| 136 | TGTAGTATATTGTTGAATTGTGAT | |
| 137 | ATAGTGATTGTATGTATGATAAAG | |
| 138 | TTAGTGATTGATGTATATTGAAAG | |
| 139 | GTAAGATTATGAGTTATGATGTAA | |
| 140 | GTTATGAAATTGTTAGTGTAGATT | 99 |
| 141 | GTTAGATTTGTAGTTTAAAGATAG | 100 |
| 142 | TTAGTGATTGAAATGATGTAGATT | |
| 143 | AAAGTGTAGTTATTAGTTAGTTAG | |
| 144 | AAAGAAAGTGTATGATGTTATTAG | |
| 145 | GATTGTATATTGTGTATGATGATT | |
| 146 | TTGAGATTGTTATGATATGAGTAT | |
| 147 | ATGAGTATGATTGTTATGATGTTT | |
| 148 | TGATTTAGTGAAATTGTGTATTAG | |
| 149 | TGAATGTATGTAGTATGTTTGTTA | |
| 150 | GTTAGTATTGATGATTATGAGTTA | |
| 151 | GTATATTGTGATTTAGTTGAGATT | |
| 152 | GTTAGTTTAAAGTTGAGATTGTTT | |
| 153 | GTATATTGTTAGATGAGATTTGTA | |
| 154 | TGATGTATGTTAGTTTATGAATGA | |
| 155 | TGTAGTATGTAATGTAGTATTTGA | |
| 156 | ATGAGTTATGTATTGAGTTAGTAT | |
| 157 | TGTATGATGATTATAGTTGAGTAA | |
| 158 | ATTGATGAATGAGTTTGTATAAAG | |
| 159 | TTGAGTTTATGATTAGAAAGAAAG | |
| 160 | TGATATTGATGAGTTAGTATTGAA | |
| 161 | ATAGAAAGTGAAATGAGTATGTTA | |
| 162 | TTGATGTAGATTTGATGTATATAG | |
| 163 | TTGAGATTATAGTGTAGTTTATAG | |
| 164 | TGATGTTAGATTGTTTGATTATTG | |
| 165 | TGTATTAGATAGTGATTTGAATGA | |
| 166 | GATTATGATGAATGTAGTATGTAA | |
| 167 | TGAATGATTGATATGAATAGTGTA | |
| 168 | GTAATGATTTAGTGTATTGAGTTT | |
| 169 | TGTAGTAATGATTTGATGATAAAG | |
| 170 | TGAAGATTGTTATTAGTGATATTG | |
| 171 | GTATTTGAATGATGTAATAGTGTA | |
| 172 | GTATATGATGTATTAGATTGAAAG | |
| 173 | AAAGTTAGATTGAAAGTGATAAAG | |
| 174 | GTAAGATGTTGATATAGAAGATTA | 9 |
| 175 | TAATATGAGATGAAAGTGAATTAG | |
| 176 | TTAGTGAAGAAGTATAGTTTATTG | 13 |
| 177 | GTAGTTGAGAAGATAGTAATTAAT | |
| 178 | ATGAGATGATATTTGAGAAGTAAT | |
| 179 | GATGTGAAGAAGATGAATATATAT | |
| 180 | AAAGTATAGTAAGATGTATAGTAG | 14 |
| 181 | GAAGTAATATGAGTAGTTGAATAT | |
| 182 | TTGATAATGTTTGTTTGTTTGTAG | 28 |
| 183 | TGAAGAAGAAAGTATAATGATGAA | |
| 184 | GTAGATTAGTTTGAAGTGAATAAT | 32 |
| 185 | TATAGTAGTGAAGATGATATATGA | |
| 186 | TATAATGAGTTGTTAGATATGTTG | |
| 187 | GTTGTGAAATTAGATGTGAAATAT | |
| 188 | TAATGTTGTGAATAATGTAGAAAG | 40 |
| 189 | GTTTATAGTGAAATATGAAGATAG | 42 |
| 190 | ATTATGAAGTAAGTTAATGAGAAG | 47 |
| 191 | GATGAAAGTAATGTTTATTGTGAA | |
| 192 | ATTATTGAGATGTGAAGTTTGTTT | 48 |
| 193 | TGTAGAAGATGAGATGTATAATTA | 53 |
| 194 | TAATTTGAGTTGTGTATATAGTAG | |
| 195 | TGATATTAGTAAGAAGTTGAATAG | |
| 196 | GTTAGTTATTGAGAAGTGTATATA | 55 |
| 197 | GTAGTAATGTTAATGAATTAGTAG | 58 |
| 198 | GTTTGTTTGATGTGATTGAATAAT | |
| 199 | GTAAGTAGTAATTTGAATATGTAG | 64 |
| 200 | GTTTGAAGATATGTTTGAAGTATA | |
| 201 | ATGATAATTGAAGATGTAATGTTG | |
| 202 | GTAGATAGTATAGTTGTAATGTTA | 66 |
| 203 | GATGTGAATGTAATATGTTTATAG | 69 |
| 204 | TGAAATTAGTTTGTAAGATGTGTA | 74 |
| 205 | TGTAGTATAAAGTATATGAAGTAG | 63 |
| 206 | ATATGTTGTTGAGTTGATAGTATA | 89 |
| 207 | ATTATTGAGTAGAAAGATAGAAAG | 94 |
| 208 | GTTGTTGAATATTGAATATAGTTG | |
| 209 | ATGAGAAGTTAGTAATGTAAATAG | |
| 210 | TGAAATGAGAAGATTAATGAGTTT | |

TABLE II

| | | |
|---|---|---|
| | | No. in |
| Sequence | SEQ ID NO: | Ex 4 |

| | | |
|---|---|---|
| AAATTGTGAAAGATTGTTTGTGTA | 1 | 1 |
| GTTAGAGTTAATTGTATTTGATGA | 2 | — |
| ATGTTAAAGTAAGTGTTGAAATGT | 3 | — |
| TGATGTTAGAAGTATATTGTGAAT | 4 | — |
| TTTGTGTAGAATATGTGTTGTTAA | 5 | — |
| ATAAGTGTAAGTGAAATAAGAAGA | 6 | — |
| AAGAGTATTTGTTGTGAGTTAAAT | 7 | — |
| GTGTTTATGTTATATGTGAAGTTT | 8 | — |
| AAAGAGAATAGAATATGTGTAAGT | 9 | — |
| TATGAAAGAGTGAGATAATGTTTA | 10 | — |
| ATGAGAAATATGTTAGAATGTGAT | 11 | — |
| TTAGTTGTTGATGTTTAGTAGTTT | 12 | — |
| GTAAAGAGTATAAGTTTGATGATA | 13 | — |
| AAAGTAAGAATGATGTAATAAGTG | 14 | — |
| GTAGAAATAGTTTATTGATGATTG | 15 | — |
| TGTAAGTGAAATAGTGAGTTATTT | 16 | 2 |
| AAATAGATGATATAAGTGAGAATG | 17 | — |
| ATAAGTTATAAGTGTTATGTGAGT | 18 | — |
| TATAGATAAAGAGATGATTTGTTG | 19 | — |
| AGAGTTGAGAATGTATAGTATTAT | 20 | — |
| AAGTAGTTTGTAAGAATGATTGTA | 21 | — |
| TTATGAAATTGAGTGAAGATTGAT | 22 | — |
| GTATATGTAAATTGTTATGTTGAG | 23 | — |
| GAATTGTATAAAGTATTAGATGTG | 24 | 4 |
| TAGATGAGATTAAGTGTTATTTGA | 25 | — |
| GTTAAGTTTGTTTATGTATAGAAG | 26 | — |
| GAGTATTAGTAAAGTGATATGATA | 27 | — |
| GTGAATGATTTAGTAAATGATTGA | 28 | — |
| GATTGAAGTTATAGAAATGATTAG | 29 | — |
| AGTGATAAATGTTAGTTGAATTGT | 30 | — |
| TATATAGTAAATGTTTGTGTGTTG | 31 | — |
| TTAAGTGTTAGTTATTTGTTGTAG | 32 | — |
| GTAGTAATATGAAGTGAGAATATA | 33 | — |
| TAGTGTATAGAATGTAGATTTAGT | 34 | — |
| TTGTAGATTAGATGTGTTTGTAAA | 35 | — |
| TAGTATAGAGTAGAGATGATATTT | 36 | — |
| ATTGTGAAAGAAAGAGAAGAAATT | 37 | 7 |
| TGTGAGAATTAAGATTAAGAATGT | 38 | — |
| ATATTAGTTAAGAAAGAAGAGTTG | 39 | — |
| TTGTAGTTGAGAAATATGTAGTTT | 40 | — |
| TAGAGTTGTTAAAGAGTGTAAATA | 41 | — |
| GTTATGATGTGTATAAGTAATATG | 42 | — |
| TTTGTTAGAATGAGAAGATTTATG | 43 | 10 |
| AGTATAGTTTAAAGAAGTAGTAGA | 44 | — |
| GTGAGATATAGATTTAGAAAGTAA | 45 | — |
| TTGTTTATAGTGAAGTGAATAGTA | 46 | — |
| AAGTAAGTAGTAATAGTGTGTTAA | 47 | — |
| ATTTGTGAGTTATGAAAGATAAGA | 48 | — |
| GAAAGTAGAGAATAAAGATAAGAA | 49 | — |
| ATTTAAGATTGTTAAGAGTAGAAG | 50 | — |
| GTTTAAAGATTGTAAGAATGTGTA | 51 | — |
| TTTGTGAAGATGAAGTATTTGTAT | 52 | — |
| TGTGTTTAGAATTTAGTATGTGTA | 53 | — |
| GATAATGATTATAGAAAGTGTTTG | 54 | — |
| GTTATTTGTAAGTTAAGATAGTAG | 55 | — |
| AGTTTATTGAAAGAGTTTGAATAG | 56 | — |
| TTGTGTTTATTGTGTAGTTTAAAG | 57 | — |
| ATTGTGAGAAGATATGAAAGTTAT | 58 | — |
| TGAGAATGTAAAGAATGTTTATTG | 59 | 13 |
| ATGTGAAAGTTATGATGTTAATTG | 60 | — |
| GTTTAGTATTAGTTGTTAAGATTG | 61 | — |
| GATTGATATTTGAATGTTTGTTTG | 62 | 14 |
| TGAATTGAAAGTGTAATGTTGTAT | 63 | — |
| GATTGTATTGTTGAGAATAGAATA | 64 | — |
| AAATTTGAGATTTGTGATAGAGTA | 65 | — |
| GTAATTAGATTTGTTTGTTGTTGT | 66 | — |
| GTTTGTATTGTTAGTGAATATAGT | 67 | — |
| ATGTAGTAGTAGATGTTTATGAAT | 68 | — |
| TGTTTAAAGATGATTGAAGAAATG | 69 | — |
| TGTGATAATGATGTTATTTGTGTA | 70 | — |
| ATAGTTGTGAGAATTTGTAATTAG | 71 | — |
| ATAGATGTAAGAGAAATTGTGAAA | 72 | — |
| AGATTAAGAGAAGTTAATAGAGTA | 73 | — |
| GAAGTAAATTGTGAATGAAAGAAA | 74 | — |
| AATGTAAGAAAGAAGATTGTTGTA | 75 | — |
| TTTGATTTATGTGTTATGTTGAGT | 76 | — |
| GTATTGAGAAATTTGAAGAATGAA | 77 | — |
| GAATTGTATGAAATGAATTGTAAG | 78 | — |
| TATTGTAGAAGTAAAGTTAGAAGT | 79 | — |
| TTTATGTAATGATAAGTGTAGTTG | 80 | — |
| ATATAGTTGAAATTGTGATAGTGT | 81 | — |
| ATAAGAAATTAGAGAGTTGTAAAG | 82 | — |
| GAATTGTGAAATGTGATTGATATA | 83 | — |
| AAATAAGTAGTTTAATGAGAGAAG | 84 | — |
| GATTAAAGAAGTAAGTGAATGTTT | 85 | — |
| TATGTGTGTTGTTTAGTGTTATTA | 86 | — |
| GAGTTATATGTAGTTAGAGTTATA | 87 | — |
| GAAAGAAAGAAGTGTTAAGTTAAA | 88 | — |
| TAGTATTAGTAAGTATGTGATTGT | 89 | — |
| TTGTGTGATTGAATATTGTGAAAT | 90 | — |
| ATGTGAAAGAGTTAAGTGATTAAA | 91 | — |
| GATTGAATGATTGAGATATGTAAA | 92 | — |
| AAGATGATAGTTAAGTGTAAGTTA | 93 | 17 |
| TAGTTGTTATTGAGAATTTAGAAG | 94 | — |
| TTTATAGTGAATTATGAGTGAAAG | 95 | — |
| GATAGATTTAGAATGAATTAAGTG | 96 | 18 |
| TTTGAAGAAGAGATTTGAAATTGA | 97 | — |
| ATGAATAAGAGTTGATAAATGTGA | 98 | — |
| TGTTTATGTAGTGTAGATTGAATT | 99 | — |
| TTTAAGTGAGTTATAGAAGTAGTA | 100 | 19 |
| GATTTATGTGTTTGAAGTTAAGAT | 101 | — |
| TAGTTAGAGAAAGTGATAAAGTTA | 102 | — |
| GTAATGATAATGAAGTGTATATAG | 103 | — |
| AATGAAGTGTTAGTATAGATAGTA | 104 | — |
| TAAATTGAGTTTGTTTGATTGTAG | 105 | — |
| TAATGAAGAATAAGTATGAGTGTT | 106 | — |
| AAATGTAATAGTGTTGTTAGTTAG | 107 | — |
| AGAGTTAGTGAAATGTTGTTAAAT | 108 | — |
| GAAATAGAAATGTATTGTTTGTGA | 109 | — |
| AGTTATAAGTTTGTGAGAATTAAG | 110 | — |
| GAGTTTATAGTTAGAATATGTTGT | 111 | — |
| AGAGTTATTAGAAGAAGATTTAAG | 112 | — |
| GAGTTAATGAAATAAGTATTTGTG | 113 | — |
| ATGATGAATAGTTGAAGTATATAG | 114 | — |
| ATAGATATGAGATGAAAGTTAGTA | 115 | — |
| TATGTAAAGAAAGTGAAAGAAGAA | 116 | — |
| TGAATGTAGAAATGAATGTTGAAA | 117 | — |
| AATTGAATAGTGTGTGAGTTTAAT | 118 | — |
| AGATATTGTTTGATTAATGAAGAG | 119 | — |
| AAAGTTGTAAAGTTGAAGATAAAG | 120 | — |
| GTTAAGAGATTATGAGATGTATTA | 121 | — |
| AGAAGATATAAGAAGATTGAATTG | 122 | — |
| GTAGAAATTTGAATTGATGTGAAA | 123 | — |
| AAGAGTAGATTGATAAGTATATGA | 124 | — |
| TGATATAGTAGTGAAGAAATAAGT | 125 | 22 |
| AGATAATGATGAGAAATGAAGATA | 126 | — |
| ATGTGAAAGTATTTGTGATATAGT | 127 | — |
| AATAAGAGAATTGATATGAAGATG | 128 | 23 |
| TAAGTGTATTTAGTAGAATGAAGT | 129 | — |
| TATGTTAGATTTGTTGAGATTGAT | 130 | — |
| AGTTTGTATGAAGAGATAGTATTT | 131 | — |
| GAGAAATGTTATGTATTTAGTAGT | 132 | — |
| TATGTGAGAATGTGTTTGATTTAA | 133 | — |
| GTATGTTTGTTTATAGAATGTATG | 134 | — |
| GAGTATATAGAAGAAAGAAATTTG | 135 | — |
| ATGAGTGAAGTAAATGTAGTTATT | 136 | — |
| TTAAGAAGTGAGTTATTGTGATAT | 137 | — |
| ATGAAATGAGAATATTGTTGTTTG | 138 | — |
| GATTAATGATTATGTGAATTGATG | 139 | — |
| GAAATGTTAAAGATATGAAAGTAG | 140 | — |
| TATTGTTGATTTGATATTAGTGTG | 141 | — |
| TTTATGTTTGTGTATGTAAGTAGT | 142 | — |
| AATTGAAAGAATTGTGTGAATTGA | 143 | — |
| TGAGTTTGAATTTGTTTGAGTAAT | 144 | — |
| GATGTATAATGATGTGTGTAAATT | 145 | — |
| ATGTGAGAGAAGAATTTGTTTATT | 146 | — |
| GTGATAAAGTATTGTTGATAGAAA | 147 | — |
| GAAGTAGAATAGAAAGTTAATAGA | 148 | — |
| TTGTGTAGTTAAGAGTTGTTTAAT | 149 | 24 |
| TAGTAGTAAGTTGTTAGAATAGTT | 150 | — |
| AATTTGAAGTATAATGAATGTGTG | 151 | — |
| TAGAAATTGTAGTATTTGAGAGAA | 152 | — |
| TGTATATGTTAATGAGATGTTGTA | 153 | 25 |
| TATTTGATAAGAGAATGAAGAAGT | 154 | 26 |
| TTGAATAGTGTAATGAATATGATG | 155 | — |
| GTAGTTTGTGAATAGAATTAGTTT | 156 | — |
| AAAGATGATTGTAATTTGTGTGAA | 157 | — |
| GAAGATTGTTGAGTTAATAGATAA | 158 | — |
| AGATTATGTAGTGATGTAAATGTT | 159 | — |
| GAATTTAGATGTAGATATGAATGT | 160 | — |
| GATAGAAGTGTATTAAGTAAGTTA | 161 | — |
| TATGAATTATGAGAAGAATAGAGT | 162 | — |
| TTTGTTATGAAGTGATTTGTTTGT | 163 | — |
| GTAAAGATTGTGTTATATGAAATG | 164 | — |
| TTGTGATAGTAGTTAGATATTTGT | 165 | 28 |
| GAATTAAGATAAAGAAGAGAAGTA | 166 | — |
| GATTGTAGAATGAATTTGTAGTAT | 167 | — |
| AAATAAGAGAGAGAATGATTTAGT | 168 | — |
| AATTATGTGAATAGATTGTTGAAG | 169 | — |
| TTAAGATTTATGTGATAGTAGAGT | 170 | — |
| TTAAAGATAGTGTTTGTTGTGTTA | 171 | — |
| TATTGATTTATGAAGAGTATAGTG | 172 | — |
| AAATTTGATGAGTAGTTTAAGAGA | 173 | — |
| ATAAAGTTGTTTGATGTTTGAATG | 174 | — |
| GATTGTGATGAATAATGTTATTGA | 175 | — |
| GATGAAGAAATATGATATGAATAG | 176 | — |
| TTAAAGTTATTGAAGTGAAGTTGA | 177 | — |
| TTGTAAGAAATAGAGATTTGTGTT | 178 | — |
| GAGATTGAGTTTAAGTATTAGATT | 179 | — |
| AGTGATAATAGAATGATAAATGTG | 180 | — |
| GATAATAGTGAATTTGAGTTGTAT | 181 | — |
| AGATATTTGTAGTAGAAAGTATGT | 182 | — |
| GTTATGAATGTTGAATTTGAATGT | 183 | — |
| ATGAAAGATTTAGTTGTGAGATAT | 184 | 30 |
| AAATAGAGAAGTTATGATGTGATA | 185 | — |
| TTAGTGAGAAATGTTTAATGTGAT | 186 | — |
| TGAAGAATATGTGAAATTAGTTTG | 187 | — |
| GTTTGATAGTTTAATGAGTATTGA | 188 | — |
| GTTGTAAGTAATGATAAAGTATGA | 189 | — |
| TAAGAGTAGTAATTGTTGTTTAGA | 190 | — |
| TTTGAGAGAGTATGTATGATTATT | 191 | — |
| ATTGATTGTGAATTAGATAGAAGA | 192 | — |
| GATTAGTATTTAGTAGTAATAGAG | 193 | 31 |
| TATGTATTAGAGATATTGAAAGTG | 194 | — |
| TATGTGAAAGTAATGATAAATGAG | 195 | — |
| GTAATTAGTAATGATTTGAATGAG | 196 | — |
| GTTTATTGTAAAGATGTAAGTGAA | 197 | — |
| TAGTAGAATTGTTGTTAAAGAATG | 198 | 32 |
| TATTGTTAGTTATGTAGTGTGTAA | 199 | — |
| GAGTGAAAGTTATATGAAAGTATA | 200 | — |
| ATATAGAAGTTGATGAGTTTATGA | 201 | — |
| TTTAGAAGTAAGAATAAGTGAGTA | 202 | — |
| TGTGTATAAGATATTTGTAAGAAG | 203 | — |
| TAGAAGAGTTGTATTGTTATAAGT | 204 | — |
| GTGTTATTAGTTTAAGTTAGAGTA | 205 | — |
| AATATAGTGATGTGAAATTGAATG | 206 | — |
| TTAGAGAATAGAGTGATTATAGTT | 207 | — |
| GAAGTGAGTTAATGATTTGTAAAT | 208 | — |
| AATGTAAAGTAAAGAAAGTGATGA | 209 | 33 |
| GTTAGTTATGATGAATATTGTGTA | 210 | 34 |
| AAATGAGTTAGAGTAGAATTATGT | 211 | — |
| GATATAGAAGATTAGTTAGTGATA | 212 | — |
| ATAGTTTGTTGAGATTTATGAGTA | 213 | — |
| TAGAATAGTTAGTAGTAAGAGTAT | 214 | — |
| GAATTTGTATTGTGAAGTTTAGTA | 215 | — |
| GTAGTAAGAAGAGAATTAGATTAA | 216 | — |
| AATGTGTTATGTATGTAAATAGTG | 217 | — |
| GAATTAGTTAGAGTAAATTGTTTG | 218 | — |
| GAAATTGAAGATAGTAAGAAATGA | 219 | — |
| GTGTATTATGTGATTTATGATAGA | 220 | — |
| TATTATGAGAAAGTTGAATAGTAG | 221 | 35 |
| TATGTATTGTATTGAGTAGATGAA | 222 | — |
| GTGATTGAATAGTAGATTGTTTAA | 223 | 36 |
| AGTAAGTTGTTTGATTGAAATTTG | 224 | — |
| GAAGTTTGATTTAAGTTTAAGAAG | 225 | — |
| GAGAAGATAAATGATATTGTTATG | 226 | — |
| ATGATGAGTTGTTAATAGTTAGTT | 227 | — |
| TATGATATTTGAAGAGTGTTAAGA | 228 | — |
| GAGATGATTAAAGTGATTTATGAA | 229 | — |
| ATAGTTAAGAGTGATGAGAATAAA | 230 | — |
| TTTATTGTTAGATAAAGAGTTGAG | 231 | — |
| AGAATATTGATAGTTGAAGTTGAA | 232 | — |
| TAGTGTAAAGTGTAGATTGTAAAT | 233 | — |
| AGTAGTGATATGATTTGAATATTG | 234 | — |
| TGTATTGAATTAGAATAGTGAGAA | 235 | — |
| TGATATGAGATAGAAGTTTAATGT | 236 | — |
| GAAGAAGTAAGTATAAAGTAAATG | 237 | — |
| TTTAAGTGTGATAAGAAAGATAGA | 238 | — |
| TATTGTTGAATGTGTTTAAAGAGA | 239 | 38 |
| GAATAATGATGAGATGATTATTGA | 240 | — |
| TAGAGAAAGAGAGAATTGTATTAA | 241 | 39 |
| ATGTATAATGAGATATGTTTGTGA | 242 | — |
| AATAGATAAGATTGATTGTGTTTG | 243 | 40 |
| TTTGATGATAATAGAAGAGAATGA | 244 | — |
| AGATGAATAAGTTGTGAATGTTTA | 245 | — |
| AGATGAAAGAAAGTGTAGAATATT | 246 | — |
| TGTTAAATGTATGTAGTAATTGAG | 247 | 41 |
| TAGTAGTGTGAAGTTATTTGTTAT | 248 | — |
| AGTGAATGTTTGTAAAGAGTTTAA | 249 | — |
| GATAAATGAGAATTGAGTAATTGT | 250 | — |
| TGATGAGAAATTGTTTAAGTGTTT | 251 | — |
| AAATAAGTAGTGTGAGTAATAGTA | 252 | — |
| TATGAAATATGTGATAGTAAGAGA | 253 | — |
| ATTGTAAGAGTGATTATAGATGAT | 254 | — |
| AGAGTAAGAATGAAAGAGATAATA | 255 | — |
| TAAGTAAGTAGATGTTAAAGAGAT | 256 | — |
| AAATAGAAAGAATTGTAGAGTAGT | 257 | — |
| ATAGATTTAAGTGAAGAGAGTTAT | 258 | 42 |
| GAATGTTTGTAAATGTATAGATAG | 259 | 43 |
| AAATAGAATGAGTAGTGAAATATG | 260 | — |
| TTGAATTATGTAGAGAAAGTAAAG | 261 | — |
| TAGTAAATTGAGAGTAGTTGAATT | 262 | — |
| TGTAAAGTGTTTATAGTGTGTAAT | 263 | — |
| ATATGATTTGAGATGAGAATGTAA | 264 | — |
| AATATTGATATGTGTTGTGAAGTA | 265 | — |
| AGTGAGATTATGAGTATTGATTTA | 266 | 44 |
| TTGTATTTAGATAGTGAGATTATG | 267 | — |
| ATAGAAATGAAAGATAGATAGAAG | 268 | — |
| GATTGTATATGTAAAGTAGTTTAG | 269 | — |
| TATGAATGTTATTGTGTGTTGATT | 270 | 45 |
| GATATTAGTAGAGTAAGTATATTG | 271 | — |
| TGAGATGAATTTGTGTTATGATAT | 272 | — |
| TATGAATGAAGTAAAGAGATGTAA | 273 | — |
| GAGTGAATTTGTTGTAATTTGTTT | 274 | — |
| AGAAATTGTAGAGTTAATTGTGTA | 275 | — |
| GTGTTAATGAAAGTTGTGAATAAT | 276 | — |
| TGTGATTTGTTAAGAAGATTAATG | 277 | — |
| AGTAGTATTGTAAAGTATAAAGAG | 278 | — |
| TGATTGTTGTATAGTTATTGTGTA | 279 | — |
| GATTGTAGTTTAATGTTAAGAATG | 280 | — |
| ATGAAATAAGAAATTGAGTAGAGA | 281 | — |
| TATGATGATATTTGTTGTATGTGT | 282 | — |
| TTTAGAGTTTGATTAGTATGTTTG | 283 | — |
| AATAAGAGATTGTGATGAGAAATA | 284 | — |
| AATGAATAGAATAGAGAATGTAGA | 285 | — |
| GTAGTAGTAATTTGAATGTTTGAA | 286 | 47 |
| AGTGAGTAATTGATTGATTGTTAA | 287 | — |
| GAATAATGTTTAGTGTGTTTGAAA | 288 | — |
| ATATGAAAGTAGAGAAAGTGTTAT | 289 | — |
| TGAGTTATTGTATTTAGTTTGAAG | 290 | — |
| TAGTTGAGTTTAAAGTTGAAAGAA | 291 | — |
| TAAAGAGTGATGTAAATAGAAGTT | 292 | — |
| TGTAGTGTTTAGAGTAAGTTATTA | 293 | — |
| AGAGATTAATGTGTTGAAAGATTA | 294 | — |
| GTAATAAGTTGTGAAAGAAGATTA | 295 | — |
| GAGATGTTATAGATAATGAAAGAA | 296 | — |
| TTTAGTTGATTGTTGAATAGAGTA | 297 | — |
| ATTATTGAAAGTAGATGTTAGATG | 298 | — |
| TTTATGTGTGATTGAGTGTTTAAT | 299 | — |
| TATTTAGTTAGATAGATAGAGAGT | 300 | — |
| ATGTGTTTATGTGAAAGATTTGTA | 301 | — |
| ATAGTAATTAGAAGAGAAGAATGT | 302 | — |
| TATGAGTGATTAGAATTGTATTTG | 303 | — |
| TTAATGTATTGTTTAAAGAGTGTG | 304 | — |
| ATAGAGAATTAAGAATTGTTTGAG | 305 | — |
| GTTATAAGTAGAAATGTATAGAAG | 306 | — |
| AGTAATTAGTTTGAAATGTGTAGT | 307 | — |
| GAAAGATTATGATTGTAAAGTGAT | 308 | — |
| GTAAGATTAGAAGTTAATGAAGAA | 309 | 48 |
| GAGAATGTTGAATAAGAAGTAATT | 310 | — |
| TTAAGAGTGTTTGAATAGTGTTTA | 311 | — |
| ATAAAGAAAGAGTATGAGATTATG | 312 | — |
| AGTTATTGATTGAAGATGAGAAAT | 313 | — |
| GTTTGTGTTTGTATAAGTTGTTAA | 314 | 50 |
| TTGTATGTGAGTTTAGATTAATGA | 315 | — |
| TAGTTAAAGTATAGTTGTTTGAGT | 316 | — |
| AAATTTGTGTTGAGATTTGTATAG | 317 | — |
| TATTAGTGTTATGATAAAGAGAAG | 318 | — |
| TATAAGAAGTAATTTGAGAAGAGT | 319 | — |
| TAAGTTGAGATGTTTGTTTGATAA | 320 | — |
| GTGTAGATTTATGAATTGAGTAAT | 321 | — |
| TATAGAGAAGTGTTTAGTTGTATA | 322 | — |
| ATAAAGAAGAATAGTTGTTGTGTA | 323 | — |
| AGATTGAAATAGATTAGAAAGTTG | 324 | — |
| GTTGTTATAAGAAATAGTTTGTTG | 325 | — |
| AGAAATAGAGTAAGAGTGTTTAAA | 326 | — |
| AGAGATAGTAGTAAATAGTTATTG | 327 | — |
| AAATGATTGTGTAAGTTATGTATG | 328 | — |
| AAGAAGTAAGAGAGAAATTTGAAT | 329 | — |
| GTGTGTATTTAGTTGATAATTGAT | 330 | — |
| ATTGTTGTTGTTGAGAAATGTATT | 331 | — |
| AGATAAGTTAAAGTAAAGAGAATG | 332 | — |
| TAGTTGAAGTTAGTTTAAGTGTTA | 333 | — |
| AGTAAGAATGTAATATGATGATAG | 334 | — |
| ATGAGATTGAAAGATTTATGAATG | 335 | — |
| TGATTGAATTAGAGAGAATGTATA | 336 | — |
| AGTTAGTAAGAGAATATAGTGAAT | 337 | — |
| ATTAAGATTGTATAGTTAGTGATG | 338 | — |
| GAGATAAAGAATTGAAATAGAAGA | 339 | — |
| AGAGTAAATGTTAAGAAAGAAGTT | 340 | — |
| AAAGTTTGTTATGTGTGAAGAATT | 341 | — |
| ATTGTGTTTAAGAAATATGATGAG | 342 | — |
| TATTGAAATGAGATGTATGTAGTT | 343 | — |
| ATTTGTGTGATGTTTGAAATATGA | 344 | — |
| TAAGATAATAGTGAGAGAAATTGA | 345 | — |
| ATTTATGATTAGTGTAAGTGTTGT | 346 | — |
| GATTAAGAATAAAGTGTGAAGAAT | 347 | — |
| GTAATTGATGAAGAGTTAGTTTAT | 348 | — |
| TGTGTTATGTTATAAGAAGTGATA | 349 | — |
| AGAGAAATTGAATTTAGAAATGTG | 350 | — |
| TTATTGAATGTGAGAAAGTATTTG | 351 | — |
| TGTTAATGAGAAGATAATGATAGT | 352 | — |
| GAAAGTATTTGTTGATTATTGTTG | 353 | — |
| TAGTTTATGTAGTTAATTGTTGAG | 354 | — |
| GTTGAAAGATAGTTTGATATGTAT | 355 | — |
| TTAGAAGATAGATTATTGAGAAAG | 356 | — |
| AATAATGTTGTGAAATAGATGTGA | 357 | 56 |
| AGTAAGAAAGTTTAGTTTAGTTAG | 358 | — |
| TAGTTTAATGAGATGTTTGATATG | 359 | — |
| TTAAAGATGTTAAAGAATGAGTGA | 360 | — |
| AAAGTGTGTATATGTTAGAAAGTA | 361 | — |
| ATTAAGTTATGTGTTTATGTGTTG | 362 | — |
| TTTGAAGAAGTGTTTGTATTATGT | 363 | — |
| TGTTAAGAAGTTTAGTTAAAGTTG | 364 | — |
| TTTAAGTATAAGATTGTGTGAGAT | 365 | — |
| AGATATTTGATAGATAGAAGAAAG | 366 | — |
| ATTTAGAGTTGTAAGAAGATATTG | 367 | — |
| GAGAAATTGTAATTGTTAGAGTAT | 368 | — |
| GAAGTATATGTTAAGATGTAATAG | 369 | — |
| AATATTGAAGATGTAGTGAGTTAT | 370 | — |
| GAGTTTAGAAATGATAAAGAATTG | 371 | — |
| TAAGAAATGAGTTATATGTTGAGA | 372 | 60 |
| TTGATATAAGAAGTTGTGATAAGT | 373 | — |
| AAGTGTTTAATGTAAGAGAATGAA | 374 | 61 |
| GTTGTGAGAATTAGAAATAGTATA | 375 | — |
| TTTAGTTTGATGTGTTTATGAGAT | 376 | — |
| GTAATTGAAAGTATGAGTAGTAAT | 377 | — |
| TAGTTGAATAAGATTGAGAGAAAT | 378 | — |
| TTAAGTGAAGTGTTGTTTATTGAA | 379 | — |
| ATTGATTTGTTGAAATAAGTGTTG | 380 | — |
| TGAATTGTTGATAAGTTATGAAGA | 381 | — |
| GTTTGTTATTGAGTAAGTTGAATT | 382 | — |
| TGATTTAGTATGTATTAGAGTTGA | 383 | — |
| TAAATAGAGATGAGAATAAGAAAG | 384 | — |
| AGAATGTTATATGTAGAGAAATTG | 385 | — |
| ATTTATGTAGTTGAGAGTGATAAA | 386 | — |
| GTAAAGATAGTTTGAGTAATTTGA | 387 | — |
| GAAATAGTATAATGTTAAGTGAGA | 388 | — |
| ATTGTATATTGTGTTGAAGAAAGT | 389 | — |
| GAGTTAAGTGTAAATGAAATGTAA | 390 | — |
| ATAGATTGTGTGAAAGAAAGAATT | 391 | — |
| TTAATAGAAGTTTGTAGTATGATG | 392 | — |
| TTGTATGTGAGAATAAAGTTTAGT | 393 | — |
| GTGATTAGATATGATGATATGAAT | 394 | — |
| TGAAGAAGAATTTAGATTTGTAAG | 395 | — |
| TGTATGATTATTGATTAGTGTGTT | 396 | — |
| TGTGAAAGAGAATGATAGATATTT | 397 | — |
| AATTGAAATGAGTGTGTTTAAGAA | 398 | — |
| ATTATAGAGTTAGTTTAGAATGAG | 399 | — |
| AAAGATAGAAATTGAGTGTATGAT | 400 | — |
| GTAGTTTGTTAATGTTGTATAATG | 401 | — |
| AGAGATATTAGAATGTAAGAATAG | 402 | 64 |
| AGAAGTTTGAAATATGATAGAATG | 403 | — |
| TAGAATGTAAAGTTTAGTATAGAG | 404 | — |
| AGTAGATGTATGTTAATGTGAATA | 405 | — |
| TGAAAGTGAAATATGAAATGTTGT | 406 | — |
| ATAGTATATTGAGTTTGTATGAAG | 407 | — |
| GAAGAAATGTTTGTAGAATAAGTA | 408 | — |
| AATGAGTATTGAAGAAATGTATAG | 409 | — |
| GTGATAGAATTTGTGTTTAATGAA | 410 | 66 |
| TGTAGTATGAAGAATAATGAAATG | 411 | — |
| ATAGAAGTTAATGATAATTGTGTG | 412 | — |
| GTGATTGTAAGTAAGTAAAGATAA | 413 | — |
| TATGTAGTTTGTGTTATTTGAAGA | 414 | — |
| TGAGTAAGTTTGTATGTTTAAGTA | 415 | 67 |
| TAAATGTATGAGTGTGTAAAGAAA | 416 | — |
| GTAAGAGTATTGAAATTAGTAAGA | 417 | 68 |
| GTTGAGTGTAAAGATTATTGATAA | 418 | — |
| AGTATGAGTTATTAGATAAAGTGA | 419 | — |
| ATTTGTTATAGAGTTGTGTTGTAT | 420 | — |
| TAATTAGTAGTGTGTTGAAATTTG | 421 | — |
| TGTATTGAGATTGTTATTGTATTG | 422 | — |
| GTTATTAGAAGAGATAATTGAGTT | 423 | — |
| TTGAGTTGTGATTAAGTAGTATAT | 424 | — |
| GATAGTATAATGATTGAAGTAATG | 425 | — |
| GTGAAAGATATTTGAGAGATAAAT | 426 | — |
| AGTTATGATTTGAAGAAATTGTTG | 427 | — |
| GTAAGTATTTGAATTTGATGAGTT | 428 | — |
| TAATAGTGTTATAAGTGAAAGAGT | 429 | — |
| AAATGAATTGATGTGTATATGAAG | 430 | — |
| AGAAAGTGAGTTGTTAAGTATTTA | 431 | — |
| TTTATGTGTGAATTGTGTATATAG | 432 | — |
| GTAATATGATAGAAATGTAAAGAG | 433 | — |
| GAGAATTGTTTAAAGATAGTTGTA | 434 | — |
| GAATTTGTTAAGAATGAGTTTGAT | 435 | — |
| ATAGTGATGATTAAAGAGAATTTG | 436 | — |
| ATAGATGTTTAGTTGAGATTATTG | 437 | — |
| AAGAGTGTAAATAGAAAGTGATAT | 438 | — |
| TGTGTATTGATTGTTGAGATAAAT | 439 | — |
| TAGTATAGTGAGAAAGAGTTAAAT | 440 | — |
| AAAGATAAGAAAGAGATGATGTTT | 441 | — |
| GAAGTTATTGAAATAGAGAAGTAT | 442 | — |
| ATGTATGTATAGAAAGAGTAAATG | 443 | — |
| GATGTTTGTAAAGATTGAAATTGA | 444 | — |
| AATTTAGAGAGTATTTGTGTTGTA | 445 | — |
| AATTTGTTTGAAAGAAAGTAAGTG | 446 | — |
| AAAGAGTAGTGTTATTGTTAGATA | 447 | — |
| GTATGTTGTATATGTTGTTGATAT | 448 | — |
| GTAGAATTTGTTGAGTATTTGTAA | 449 | — |
| ATGAATTTAGTTAGTGTAAGAAAG | 450 | — |
| ATGATAAGAAATGTTGATGAAGTA | 451 | — |
| TTGATGATGAAGATAATGTAGATA | 452 | — |
| AGATGATATGATATAGATTAGATG | 453 | — |
| TTGAAAGTTAGAAAGATAGATGTT | 454 | — |
| GTTTAATGTTAGTTAGAAAGTAAG | 455 | — |
| GAGATTTAAGTTTGAAGTGAAATA | 456 | — |
| TTTGTTAGTAGTTGTTATAAGAGA | 457 | — |
| TATGAGAATAGTTTGTTAGTGAAT | 458 | — |
| TTGAAAGTTTAAAGAAGAGATAAG | 459 | — |
| AAGTGAGTTGAAATGAAATATGTT | 460 | — |
| GTTAGAAATGAAATGAGTAGTTAT | 461 | — |
| TAAGTATTGTATTTGTGTGTGTAT | 462 | — |
| TGTATTAGTAAAGAAGAGAGAATA | 463 | — |
| GAGAAGAGAAATAAGTTGAAATAA | 464 | — |
| GTAAAGTAGAAATAGAATTGAGTT | 465 | — |
| GTGTGTTATTTGTTTGTAAAGTAT | 466 | 69 |
| TTTGATGTATGAATATAGTATGAG | 467 | — |
| AAGATTGTGTGAATAGTTGAAATT | 468 | — |
| TATAAAGTTTGAAGATGAGTGATA | 469 | — |
| AGATAAAGAGATTTAAGATGTATG | 470 | 71 |
| GAAGAATTAAGTTGAGAATTAAGA | 471 | — |
| TAGAGAAATTTGATAAAGAAAGAG | 472 | — |
| AAAGTTTATGAAGTTATTGAGTAG | 473 | — |
| AAATAGTGTAAGTAAAGAGATGAT | 474 | — |
| TATGATGATTTAGTTATAAGAGTG | 475 | — |
| TAGATAAATGTTATGATGAGTAAG | 476 | — |
| AGATTGATTGTGATGATTTGTATA | 477 | — |
| TTAAGAAGAATTGTATATGAGAGT | 478 | 73 |
| GTAGAATGTTTAGAGTTGAATATA | 479 | — |
| GAGAAATAGTAAGAAGTAAATAGA | 480 | — |
| ATTGAAGTTGTTATGTGAAGATTT | 481 | — |
| TAAATGTTGTGTAGAGTAATTAGA | 482 | — |
| AAATAAGAGTTTGAGAAGTTGTTT | 483 | — |
| AGTTGTAATAAGAAGTGATTTAAG | 484 | — |
| GTTAGAATGTATATAGAGTTAGAT | 485 | 74 |
| TTGATATTGAAAGAGAAAGTTATG | 486 | — |
| TTAAAGAGAGAAATGTTTGATTAG | 487 | — |
| TGTGAATTTGAGTATTAGTAAGAA | 488 | — |
| TAATTTGAATGTGAAAGTTGTTAG | 489 | — |
| ATGTGTTTGAAAGATGATGATTTA | 490 | — |
| AAGTTATGTTGATATTGAGTGAAA | 491 | — |
| TAGATAAAGAAGATAGAGATTTAG | 492 | — |
| GATGAATGTAGATATATGTAATGA | 493 | — |
| GAAGAATAGTTTATGTAAATGATG | 494 | — |
| GTAGTATATAGTTAAAGATGAGTT | 495 | — |
| GTTATTTGTGTATGATTATGATTG | 496 | — |
| AGAGATTAGAAATTGAGAGAATTA | 497 | — |
| GTATGATAGAGTTTATAGTGATAA | 498 | — |
| GTTAGAAAGAATGAAATTGAAGTA | 499 | — |
| AAGAATGAGAATATAGAGATGAAT | 500 | — |
| AAAGAGAATAGTGTTTAAGAAGAT | 501 | — |
| GATGTGTTATTGATAGAAATTAGA | 502 | — |
| TAGAGTTATAGAGATATTGTATGA | 503 | — |
| GAGAGTTGAATAAGTTAAAGATAT | 504 | — |
| AGATATGAAATAGATTGTTAGAGA | 505 | — |
| GAGTGAATAGAAAGATATGTTAAT | 506 | — |
| AAAGAGATATTGAAGAGAATAAAG | 507 | — |
| GTTATAGAATAAGTTGTAAAGTGT | 508 | — |
| TGATAGTATGATAATGTGTTTATG | 509 | — |
| TTTGTTGTTAAGTATGTGATTTAG | 510 | 77 |
| TAAAGTGTTGTGTTAAAGATTAAG | 511 | — |
| TGTGTTTGATTGATTAATGTTATG | 512 | — |
| ATTAATGAATGAGTGTTGTAATGT | 513 | — |
| TAGATGTTTGTGAGTTTGATATTA | 514 | — |
| GAATGAATAGTAATAGATGATTTG | 515 | — |
| AATAGTGTGTTGTTATATGATTAG | 516 | — |
| TAGATTAGAAGATGTTGTGTATTA | 517 | — |
| AATGTGTGTGTTAAATGAATTTGT | 518 | — |
| GAATTAAGTATATGAGTGTAGAAA | 519 | — |
| TTATTGTGTGTAAGTAGTGTAAAT | 520 | — |
| GTAGTAAAGAGAATTGTTTAGTAT | 521 | 80 |
| AAGTTTGTAAGAAGTAGTTGAATA | 522 | — |
| AGTTATAGTATAGTAGTATAGAGA | 523 | — |
| GAAAGAAATGTGTATAGTTTAATG | 524 | — |
| TTGTGAGTAATGAATGATGTATTA | 525 | — |
| GTAGAGTTGTAAATAGAGAATAAA | 526 | — |
| ATTAATGTAGATTGTAAGAGATAG | 527 | — |
| TTAGTGTGTTTGTAGATAGAATTA | 528 | — |
| AGAGAGTTTGTGTATATGTATAAA | 529 | 81 |
| TTAAGTTTAGTGAGATTTGTTAAG | 530 | — |
| ATGAAGTTTATTGAATAGTAGTGA | 531 | — |
| ATATTTGTGTTGTATGTTTGTGAA | 532 | — |
| AAAGTGTTTATAGAAGATTTGATG | 533 | — |
| AAGAGATATGATTTGTTAGTTGTA | 534 | — |
| AAGAAGAAATGAGTGATAATGTAA | 535 | — |
| TAGTGTTTGATATGTTAAGAAGTT | 536 | — |
| GTAGAAAGTGATAGATTAGTAATA | 537 | — |
| GATAAATGTTAAGTTAGTATGATG | 538 | — |
| AGATTAGAAGAATTGTTTAGAATG | 539 | — |
| ATATTTGAGAAGTGTGAAATGAAT | 540 | — |
| TGAGTAAATAGTTTATGAGTAGTA | 541 | — |
| TTAGAGAGTAGATAAAGATTTGAT | 542 | — |
| ATTGTTTAAGTTGTTGATAAGATG | 543 | — |
| GTTGTAAAGTTAAAGTGTGAATTT | 544 | — |
| ATAGATTGTGTGTTTGTTATAGTA | 545 | — |
| GTAAGTTATTGAGAATGATAATAG | 546 | — |
| TAGATTAGTTGATAAGTGTGTAAT | 547 | 83 |
| AAATGTAAATGAAGAGTGTTTGTT | 548 | — |
| GATAGAAGAAATGTATATAGTGAT | 549 | — |
| TATAGAGTGTATGTTATGATAAAG | 550 | — |
| TATGAAGTGATAAGATGAAGAATT | 551 | — |
| TGTTGAGAATAGTAAGAGAATTTA | 552 | — |
| TAGATAATGTGAAGTAATAAGTGA | 553 | 84 |
| GTATTATGATGATAGTAGTAAGTA | 554 | — |
| AGATATGATTTAGTATTGAATGTG | 555 | — |
| AATTAAGTTTGTAGAGTGATTTGA | 556 | — |
| AAGAAATAGATGTAGTAAGATGTT | 557 | — |
| TTGAGAAGTTGTTGTAATAAGAAT | 558 | — |
| AGTGTGAAATAGTGAAAGTTTAAA | 559 | — |
| TTTATGTAGTAGATTTATGTGAAG | 560 | — |
| ATTAATGAGAAATTAGTGTGTTAG | 561 | — |
| ATGTTAATAGTGATAGTAAAGTGA | 562 | — |
| TATGTTGATAAATGATTATGAGTG | 563 | — |
| TTATTAGAGTTGTGTGTGATATAT | 564 | — |
| TGTTGTTATGATTGAGTTAGAATA | 565 | — |
| AATTTGAGTTAAGAAGAAGTGTAA | 566 | — |
| AAAGATAAAGTTAAGTGTTTGTAG | 567 | 88 |
| TGTTGAGATGATATTGTATAAGTT | 568 | — |
| TAAATAGTGAATGAGTTATAGAGT | 569 | — |
| ATAGATGTTATGATAGTTAGTTAG | 570 | — |
| GTTAAGTGAAGATATGTATTGTTA | 571 | — |
| TAAGAAAGTAAAGTTTGTAGATGT | 572 | — |
| AAGAGAAAGTTTGATTGAATAAAG | 573 | — |
| ATATTAGATGTGAGTTATATGTGT | 574 | — |
| AGTTTGAGTTTAGTATTGTGAATA | 575 | — |
| ATGTTAAATGAGAGATTGTGTATA | 576 | — |
| TAAATGTTGTGATTATTGTGAGAT | 577 | — |
| TAAGAATTGAAGTAAGAGTTATTG | 578 | — |
| AGAGATAGAATTAAGTTTGTTGAT | 579 | — |
| GAAGAATGTTAAGAAATATGTAAG | 580 | — |
| TATTTGTGATTAAGAAGTTGAGAA | 581 | — |
| AGTTAGAATTTGTGTAGTAGAATT | 582 | — |
| AAGTTTATTGTTGATGTTGTATTG | 583 | — |
| GAATGAGTTTAAGAGTTTATAGTA | 584 | — |
| AGTGAAGATTGTATGTAGTATAAA | 585 | — |
| AGTTGAAATGAGTATTAAGTAATG | 586 | — |
| ATGTGTTATTTGAGATGAGTAATT | 587 | — |
| AAATAGTGTTGTTGAAGTTGTTAT | 588 | — |
| GTAGAGAAAGATATATGTAGTTTA | 589 | — |
| GAGAGTATTTGATGAATGATTATA | 590 | — |
| GAGTATAAGTTTAGTGTATATTGA | 591 | — |
| ATAATGTGATTATTGATTGAGAGA | 592 | — |
| TTAGTTGTTATGTGAGAGTAATAA | 593 | — |
| AAATGAGTATATTGAATTGTGATG | 594 | — |
| AATTAGAAGTAAGTAGAGTTTAAG | 595 | 3 |
| TGTAAGTTTAAAGTAAGAAATGTG | 596 | 5 |
| GAAATGATAAGTTGATATAAGAAG | 597 | — |
| AATGAGTAGTTTGTATTTGAGTTT | 598 | — |
| AGTGAATGTAAGATTATGTATTTG | 599 | 6 |
| GTAATTGAATTGAAAGATAAGTGT | 600 | 8 |
| TATGTTTAAGTAGTGAAATAGAGT | 601 | — |
| GTATTGAAATTGAATTAGAAGTAG | 602 | — |
| AATATGTAATGTAGTTGAAAGTGA | 603 | — |
| TGAATATTGAGAATTATGAGAGTT | 604 | — |
| TAGTGTAAATGATGAAGAAAGTAT | 605 | — |
| GTATGTGTAAAGAAATTTGATGTA | 606 | — |
| AATTGTTTGAAAGTTTGTTGAGAA | 607 | — |
| AATTGTTTGAGTAGTATTAGTAGT | 608 | — |
| TAATTGAGTTTGAATAAGAGAGTT | 609 | — |
| TGTTGATTGTAAGTGTTTATTGTT | 610 | — |
| GAAATTTGTGAGTATGTATTTGAA | 611 | — |
| TAAGAATGAATGTGAAGTGAATAT | 612 | — |
| TAATGTGAAGTTTGTGAAAGATAT | 613 | — |
| TTGTATATGAAAGTAAGAAGAAGT | 614 | — |
| TAGAGAGAAGAAGAAATAAGAATA | 615 | — |
| ATTTGAAATGTTAATGAGAGAGAT | 616 | — |
| TTGTGTGTATATAGTATTAGAATG | 617 | — |
| ATTGTTAGTATTGATGTGAAGTTA | 618 | — |
| TGTTTGTATTTGAATGAAATGAAG | 619 | — |
| TGTTAGATTGTGTTAAATGTACTT | 620 | — |
| TATAGAGTATTGTATAGACAGAAA | 621 | — |
| AAATAGTAAGAATGTACTTGTTGA | 622 | — |
| TGAGTGTGATTTATCATTAAGTTA | 623 | — |
| ACAATTTGTTGTACTGTTATGATT | 624 | — |
| GATTGAAGAAAGAAATAGTTTGAA | 625 | — |
| GATAATAGAGAATAGTAGAGTTAA | 626 | — |
| GATTGAAATTTGTAGTTATAGTGA | 627 | — |
| GATTTAAGAAGATGAATAATGTAG | 628 | — |
| TTTGAGAGAAAGTAGAATAAGATA | 629 | — |
| GATTAAGAGTAAATGAGTATAAGA | 630 | — |
| TTTGATAGAATTGAAATTTGAGAG | 631 | — |
| TGAAGAAGAGTGTTATAAGATTTA | 632 | — |
| GTGAAATGATTTAGAGTAATAAGT | 633 | — |
| AAATAAGAATAGAGAGAGAAAGTT | 634 | 9 |
| GTTGTAAAGTAATAGAGAAATTAG | 635 | — |
| AGTGATTTAGATTATGTGATGATT | 636 | — |
| AGAGTATAGTTTAGATTTATGTAG | 637 | — |
| ATGATTAGATAGTGAAATTGTTAG | 638 | — |
| ATGAAATGTATTAGTTTAGAGTTG | 639 | — |
| ATATTGAGTGAGAGTTATTGTTAA | 640 | — |
| AGATGTGTATTGAATTAAGAAGTT | 641 | — |
| TAATGTGTTGATAGAATAGAGATA | 642 | — |
| AAATTAGTTGAAAGTATGAGAAAG | 643 | 11 |
| TTTAGAGTTGAAGAAATGTTAATG | 644 | — |
| GATTGTTGATTATTGATGAATTTG | 645 | — |
| TGTTGTTGTTGAATTGAAGAATTA | 646 | — |
| ATTAAGTAAGAATTGAGAGTTTGA | 647 | 12 |
| GTATGTTGTAATGTATTAAGAAAG | 648 | 15 |
| TAGTTGTGATTTATGTAATGATTG | 649 | — |
| TGATAATGAAAGTTTATAGAGAGA | 650 | — |
| GTAAGATTGTTTGTATGATAAGAT | 651 | — |
| TTGAATTAAGAGTAAGATGTTTAG | 652 | — |
| AAGTGTTTGTTTAGAGTAAAGATA | 653 | — |
| AGAGAGATAAAGTATAGAAGTTAA | 654 | — |
| ATTATGAATAGTTAGAAAGAGAGT | 655 | — |
| TTGTTGATATTAGAGAATGTGTTT | 656 | — |
| TTTATTGAGAGTTTGTTATTTGTG | 657 | — |
| AGTGTTAAGAAGTTGATTATTGAT | 658 | — |
| GAGAAATGATTGAATGTTGATAAT | 659 | — |
| GATAAGTATTAGTATGAGTGTAAT | 660 | — |
| TTTGATTTAAGAGTGTTGAATGTA | 661 | 16 |
| AAGTTAGTAAATAGAGTAGAAAGA | 662 | — |
| GTAAAGTATGAATATGTGAAATGT | 663 | — |
| TAATAAGTGTGTTGTGAATGTAAT | 664 | — |
| AAAGATTTAGAGTAGAAAGAGAAT | 665 | — |
| TTAGTTTGAGTTGAAATAGTAAAG | 666 | — |
| TAATAGTATGAGTAAGATTGAAAG | 667 | — |
| GAAGATTAGATTGATGTTAGTTAA | 668 | — |
| TAAAGAGAGAAGTTAGTAATAGAA | 669 | — |
| TAAGTATGAGAAATGATGTGTTAT | 670 | — |
| GAGTTTGTTTGTTAGTTATTGATA | 671 | — |
| AAGTAAAGAAATGTTAAGAGTAGT | 672 | — |
| ATGAGAATTGTTGTTGAAATGTAA | 673 | — |
| TTAGATTAGAGTAGTAGAAGAATA | 674 | — |
| TAGTGATGAAGAAGTTAGAAATTA | 675 | — |
| TAATGTAGTAATGTGATGATAAGT | 676 | — |
| TTGAGAAAGAATAAGTAGTGTAAA | 677 | — |
| TAATGAGTGAGATTATAGATTGTT | 678 | — |
| GTATAAGAAATGTGTGTTTGATTA | 679 | — |
| GTGAATGTGTTAATGAAGATATAT | 680 | — |
| GAAAGTTATTAGTAGTTAAAGATG | 681 | — |
| TAGAATTGTGTTTGATAAGTGATA | 682 | — |
| TGATTTAGATTGAGAGTTAAATGA | 683 | — |
| ATTATTGAGTTTGAATGTTGATAG | 684 | — |
| ATAGTAGTTATGTTTGATTTAGTG | 685 | — |
| ATAGAAGAAGAATAAAGTTAGAGA | 686 | — |
| GATGTTGAAAGTAATGAATTTGTA | 687 | — |
| GAGATTGATAGTAGAAATGATAAA | 688 | — |
| TGAGAGAATAAAGTATGAATTTGA | 689 | — |
| TATAAAGATGATGTGAATTAGTAG | 690 | — |
| TTATGTAAGAATGTTTGAGAGAAA | 691 | — |
| AGTAAATGATGAATGATATGATGA | 692 | — |
| GAAATTTGTGTTAAAGTTGAATGA | 693 | — |
| GATGAATGATTGTGTTTAAGTATA | 694 | — |
| GAAATAAGTGAGAGTTAATGAAAT | 695 | — |
| TGTTGAAATAGTTATTAGTTTGTG | 696 | — |
| TTTGAGAGTATATTGATATGAGAA | 697 | — |
| ATTGTGTGTAAAGTAAGATTTAAG | 698 | — |
| TATAGTTTGAAGTGTGATGTATTT | 699 | — |
| GTGAAGTTATAGTGTATAAAGAAT | 700 | — |
| GTATGTTGAATAGTAAATAGATTG | 701 | — |
| TTAGAAAGTGTGATTTGTGTATTT | 702 | — |
| TTTAGTAATATGTAAGAGATGTGA | 703 | — |
| AGTATGTATAGATGATGTTTGTTT | 704 | — |
| ATTTAAGTAAAGTGTAGAGATAAG | 705 | 20 |
| ATTTGTGTTGAATTGTAAAGTGAA | 706 | — |
| ATGTTATTAGATTGTGATGAATGA | 707 | — |
| TAGTAGTAGAATATGAAATTAGAG | 708 | — |
| TTTAATGAGAAGAGTTAGAGTATA | 709 | — |
| AAAGTTTAGTAGAGTGTATGTAAA | 710 | — |
| ATATATGATAGTAGAGTAGATTAG | 711 | — |
| TGAGAAGTTAATTGTATAGATTGA | 712 | — |
| TATAGAGATGTTATATGAAGTTGT | 713 | — |
| AAATTTGTTAAGTTGTTGTTGTTG | 714 | — |
| TTGTTGAAGATGAAAGTAGAATTA | 715 | — |
| AAGAGATAAGTAGTGTTTATGTTT | 716 | — |
| AATAAGAAGAAGTGAAAGATTGAT | 717 | — |
| TAAGTTAAAGTTGATGATTGATAG | 718 | — |
| ATATAAGATAAGAGTGTAAGTGAT | 719 | — |
| GTTAAATGTTGTTGTTTAAGTGAT | 720 | — |
| GAGTTAAGTTATTAGTTAAGAAGT | 721 | — |
| TATTAGAGTTTGAGAATAAGTAGT | 722 | 21 |
| TAATGTTGTTATGTGTTAGATGTT | 723 | — |
| GAAAGTTGATAGAATGTAATGTTT | 724 | — |
| TGATAGATGAATTGATTGATTAGT | 725 | — |
| ATGATAGAGTAAAGAATAAGTTGT | 726 | — |
| AGTAAGTGTTAGATAGTATTGAAT | 727 | 27 |
| ATGTAGATTAAAGTAGTGTATGTT | 728 | — |
| TTATTGATAATGAGAGAGTTAAAG | 729 | — |
| ATTTGTTATGATAAATGTGTAGTG | 730 | 29 |
| TTGAAGAAATAAGAGTAATAAGAG | 731 | — |
| TGTGTAATAAGTAGTAAGATTAGA | 732 | — |
| ATGAAAGTTAGAGTTTATGATAAG | 733 | 37 |
| ATTAGTTAAGAGAGTTTGTAGATT | 734 | — |
| TGTAGTATTGTATGATTAAAGTGT | 735 | — |
| AGTTGATAAAGAAGAAGAGTATAT | 736 | — |
| GTAATGAGATAAAGAGAGATAATT | 737 | — |
| TGTGTTGAAGATAAAGTTTATGAT | 738 | — |
| AAGAAGAGTAGTTAGAATTGATTA | 739 | — |
| GAATGAAGATGAAGTTTGTTAATA | 740 | — |
| AAATTGTTGAGATAAGATAGTGAT | 741 | — |
| TGATTGTTTAATGATGTGTGATTA | 742 | — |
| ATGAAGTATTGTTGAGTGATTTAA | 743 | — |
| GTGTAAATGTTTGAGATGTATATT | 744 | 46 |
| AATTGATGAGTTTAAAGAGTTGAT | 745 | — |
| TTTGTGTAATATGATTGAGAGTTT | 746 | — |
| GTAGTAGATGATTAAGAAGATAAA | 747 | — |
| TTTAATGTGAAATTTGTTGTGAGT | 748 | — |
| GTAAAGAATTAGATAAAGAGTGAT | 749 | — |
| AATAGTTAAGTTTAAGAGTTGTGT | 750 | — |
| GTGTGATGTTTATAGATTTGTTAT | 751 | — |
| GTATAGTGTGATTAGATTTGTAAA | 752 | 49 |
| GTTGTAAGAAAGATATGTAAGAAA | 753 | — |
| ATATTAGATTGTAAAGAGAGTGAA | 754 | — |
| GAGTGATATTGAAATTAGATTGTA | 755 | — |
| TAAGAAGTTAAAGAAGAGAGTTTA | 756 | — |
| GATGTTAGATAAAGTTTAAGTAGT | 757 | — |
| GTGATTGTATGAGAAATGTTAAAT | 758 | — |
| TGATTATTGTAAGAAAGATTGAGA | 759 | — |
| AAGAATTGTGTAAGTTTATGAGTA | 760 | — |
| TTGTATTTAGAAGATTTGTAGATG | 761 | — |
| TATATGTTTGTGTAAGAAGAAATG | 762 | — |
| GATAATGTGTGAATTTGTGAATAA | 763 | — |
| TTAGAAATGTGAGATTTAAGAGTT | 764 | — |
| AGTGTAGAATTTGTATTTAGTTGT | 765 | — |
| TAGTTAAGATAGAGTAAATGATAG | 766 | — |
| GAAGTGATATTGTAAATTGATAAG | 767 | — |
| GTAATTGTGTTAGATTTAAGAAGT | 768 | — |
| TGATATTTGTGAATTGATAGTATG | 769 | — |
| AAGTAAAGAGATATAGTTAAGTTG | 770 | — |
| ATTAGTTAAGTTATTTGTGAGTGA | 771 | — |
| AGATGAAGTAGTTTATGAATTAGA | 772 | — |
| TGAGTTAGTTAAGTGATAGTTAAA | 773 | — |
| TTATTGTAGATTTAGAGAAGATGA | 774 | — |
| TATTTGTGTTTGTTGATTAGATAG | 775 | — |
| GTATAATGTGTGTGAAAGTTATAA | 776 | — |
| TATATGTTGAGTATAAAGAGAGAA | 777 | — |
| TTAGTTAGTTTAAAGATTGTGAGT | 778 | — |
| TTTAGAATAAGTGATGTGATGAAA | 779 | — |
| AGAGTAATGTGTAAATAGTTAGAT | 780 | — |
| TGTGATAAAGAGAAATTAGTTGTT | 781 | — |
| GAATTTAGTGAATGTTTGAGATTA | 782 | — |
| TGTGATGTGTAAGTATATGAAATT | 783 | — |
| TTGTGAATGATTAATGAATAGAAG | 784 | 51 |
| AATGTTGTTTAGATTGAGAAAGTT | 785 | — |
| AGATTGTGTTAGTATTAGTATAAG | 786 | — |
| TTGATGTATTAGAAAGTTTATGTG | 787 | — |
| TATGATTGTGTGTTAGAGAATTTA | 788 | — |
| TAGTGTAGATATTTGATAGTTATG | 789 | 52 |
| AGTTTAATGTGTTTAGTTGTTATG | 790 | — |
| TGTGTAAAGTAGAAAGTAAAGATT | 791 | — |
| GTTATGATATAGTGAGTTGTTATT | 792 | 53 |
| TTTGATTGAATGTTAATAGTGTGT | 793 | — |
| AGAGTATTAGTAGTTATTGTAAGT | 794 | 54 |
| TAAGTAGAAAGAAGAAGATATTTG | 795 | — |
| AGAAAGAGAATTATGTAATGAAAG | 796 | — |
| TTAGATTTGTTAGTGTGATTTAAG | 797 | — |
| GATGATTAAGATATAGAGATAGTT | 798 | — |
| ATATTTGAGTGATTAAGAGTAATG | 799 | — |
| TGTATTGTGAGTTAAGTATAAGTT | 800 | — |
| AATTTAGTAGAAAGTGTTGTGTTT | 801 | — |
| GTTAGAAGATTAAGTTGAATAATG | 802 | — |
| TAAAGTATGTGAGATGATTTATGT | 803 | — |
| TGAAATGATTAAAGATGAAGATGA | 804 | — |
| TTATTAGATGTTGAGTGTTTGTTT | 805 | — |
| TAGTGTTTAAAGAGTAGTATATGA | 806 | — |
| AGTTATAAGTAAATGATGTTGATG | 807 | — |
| TTAAGAGAGAAATAAGTGTATTGT | 808 | — |
| GATATTGAAATGTGTAAATGATGA | 809 | — |
| ATGATGAATTAAGAAAGAAAGAGA | 810 | — |
| GAATAGTTTGATTTGTGTTTGTTA | 811 | — |
| AGTTGTTTAGATTTGATTTGTAAG | 812 | — |
| GTATGAGATTTGATATAAGATTAG | 813 | — |
| TTTATAGTGAGTATAGTGATGATT | 814 | — |
| TATATGTGAAGATATAAGTGTTTG | 815 | — |
| ATTGATAGATGATAGTAATTGAGT | 816 | — |
| TGATAGATGTGAAGAATTTGATTT | 817 | — |
| GAAGATATTGAAAGAATTTGATGT | 818 | 55 |
| GATGTTTAGTGTAGATATAGATTT | 819 | — |
| GAATATTGAGTTATAAGTAGTAGT | 820 | — |
| AGTGAGTAAGTAATAGAAAGATTT | 821 | — |
| GTAGAATAAGTAATTTGTGAGATA | 822 | — |
| GAGTTATTTGAGATTTAGATGTTT | 823 | — |
| GAAATGATGATTGAATTTAGAGAT | 824 | — |
| AAATAGTGTGAGAATAGTTAAGTA | 825 | — |
| ATGTGTTAAGTTGTAGAAGAATAA | 826 | — |
| ATAATGAGTTAATAGTGTAAGAAG | 827 | — |
| ATAAGAGATGTTTAAGTTAGAAAG | 828 | — |
| TGTTAGTGTTAGAAATATGAAAGA | 829 | — |
| TTTAGAAGATTGTTAGATAAGTTG | 830 | — |
| GTGTAATGTATAAGATAGTTAAGT | 831 | — |
| TATTAGAGAGAAATTGTAGAGATT | 832 | 57 |
| TAGTGAGATAAAGTAAAGTTTATG | 833 | — |
| TTGTGAAAGTTAAGTAAGTTAGTT | 834 | — |
| AAAGTGTAAGTTGAAGAATATTGA | 835 | — |
| GAATAGAGTGTTATTTGAAATAGA | 836 | — |
| TATAAGAGAGAGATAAGTAATAAG | 837 | — |
| TGAGTGAAATTGATAGAGTAAATT | 838 | — |
| GATGAATAAGTTTAAGTGAGAAAT | 839 | — |
| GTGTGATATGTTTATTGATTAAGT | 840 | — |
| TAAAGTGAGTGTAAATGATAATGA | 841 | — |
| GTAGAGTTTGATTTGAAAGAATAT | 842 | — |
| GAATATTGTTATGTTTGTTATGAG | 843 | — |
| GTGTAATAAGATGTATTGTTGTTT | 844 | — |
| TAAATTGATTGTGAGTTGAAGAAT | 845 | — |
| TGAGATAGTTATAGTTAAGTTTAG | 846 | — |
| AGTTTGTTAAGATTATGTAGAAAG | 847 | — |
| GAATGTGTAGAATAAGAGATTAAA | 848 | — |
| GTATTATGAAAGAAGTTGTTGTTT | 849 | — |
| GTGTTATAGAAGTTAAATGTTAAG | 850 | 58 |
| TTAAGAGTAGTGAATATGATAGTA | 851 | — |
| AATGTTATAAGATGAGAGTTTAGT | 852 | — |
| ATATAAGATTTGATGTAGTGTAGT | 853 | — |
| TATGTTTGTTGTTGTTAAGTTTGA | 854 | — |
| GATAGTTTAGTATAGAAGATAAAG | 855 | — |
| GTTGAATATAGAGATAGTAAATAG | 856 | — |
| AGAGAAGATTTAGTAAGAATGATA | 857 | — |
| TGAATGAGAAAGATATTGAGTATT | 858 | — |
| TGAAGATTATAGTAGTTGTATAGA | 859 | — |
| GATTAGTAGTATTGAAGATTATGT | 860 | — |
| TGAAATGTGTATTTGTATGTTTAG | 861 | 59 |
| ATTAAAGTTGATATGAAAGAAGTG | 862 | — |
| AATGTAGAGATTGTAGTGAATATT | 863 | 62 |
| TTATTTGTTGAGTGTAAATGTGAT | 864 | — |
| ATGTAATTGTGAATAATGTATGTG | 865 | 63 |
| GATTTGTATAGAGATTAGTAAGTA | 866 | — |
| AATATTGTTGTTTAGAGAAAGAAG | 867 | — |
| ATGATGATGTATTTGTAAAGAGTA | 868 | — |
| AATGTATTTGTGTGATTGTGTAAA | 869 | — |
| AGTGTTATGAAGAATAGTAAGAAT | 870 | — |
| GTTATGTAGAGATGAAAGAAATTA | 871 | 65 |
| GTTTGTATTAGATAAATGAGTTGT | 872 | — |
| TGATTTATGAGATTAAGAGAAAGA | 873 | — |
| TTTGTGTGTTATTGTAATTGAGAT | 874 | 70 |
| GATGTGTGATATGATTAAAGAAAT | 875 | — |
| AGATTATAGATTTGTAGAGAAAGT | 876 | — |
| GAAGAGTATGTAATAGTATTGTAT | 877 | — |
| TTTGTAATGTTGTTGAGTTTAAGA | 878 | — |
| AGTAAATAGTAGTATGAATAAGAG | 879 | — |
| GAATGTTGAATTGAAATATGAGTT | 880 | — |
| AGTAGTTAATTGATAGTAAGTTTG | 881 | — |
| AGTGTAAAGAAATGAATGAATAAG | 882 | — |
| TGTTAGATATTTGTGAAATGTGAA | 883 | — |
| TGTATGTTGAGTTTGAATTGTTAT | 884 | — |
| TGAGTGAATTAGTTATGTTGTTAT | 885 | — |
| GAAGAAAGAAATGAGAAAGATTAT | 886 | — |
| TTAAGTAAGTTGTGTTGATATTAG | 887 | — |
| ATGATGTGTTTGATTTGAATTGAA | 888 | 72 |
| AAGTAAGTGAAATTGTTGTTTGAA | 889 | — |
| ATGAAGTGTAAAGTTTGAAAGAAA | 890 | — |
| AGAGAGTAAGATAATTGTATAGTA | 891 | — |
| TTTATGAGATAGATGAAATAAGTG | 892 | — |
| AGAAATTAGTAGTAATGATTTGTG | 893 | — |
| GATTTGAGATTGAATGAGAATATA | 894 | — |
| GATTAGAAAGATGAATAAAGATGA | 895 | — |
| TAGATAGAAAGTATATGTTGTAGT | 896 | — |
| GAAGATAGTAAAGTAAAGTAAGTT | 897 | — |
| AAATGTGTGTTTAGTAGTTGTAAA | 898 | 75 |
| TTGTTGAAGTAAGAGATGAATAAA | 899 | — |
| TATTTGAGAGAAAGAAAGAGTTTA | 900 | — |
| TATTTAGTGATGAATTTGTGATGT | 901 | — |
| TTATAGTGATGATGATAAGTTGAT | 902 | — |
| TAAAGATAATTGTAGAAAGTAGTG | 903 | — |
| GTTTAGTATTGATATTGTGTGTAA | 904 | — |
| GTGTTGTGAATAAGATTGAAATAT | 905 | — |
| AAAGAAAGTATAAAGTGAGATAGA | 906 | — |
| TATTTGTAAGAAGTGTAGATATTG | 907 | — |
| TAGAAGATGAAATTGTGATTTGTT | 908 | — |
| ATAATAGTAAGTGAATGATGAGAT | 909 | — |
| AATGTGAATAAGATAAAGTGTGTA | 910 | — |
| ATTGAAGATAAAGATGTTGTTTAG | 911 | — |
| TGAAATAGAAGTGAGATTATAGTA | 912 | 76 |
| AGTTATTGTGAAAGAGTTTATGAT | 913 | — |
| AAATAGTAGTGATAGAGAAGATTT | 914 | — |
| AGTGTATGAAGTGTAATAAGATTA | 915 | — |
| TGATTAAGATTGTGTAGTGTTATA | 916 | — |
| AGTTTATGATATTTGTAGATGAGT | 917 | — |
| TATGTGTATGAAGATTATAGTTAG | 918 | 78 |
| GAAATTGTTGTATAGAGTGATATA | 919 | — |
| TAGAAATAGTTTAAGTATAGTGTG | 920 | — |
| TGATTTAGATGTTTATTGTGAGAA | 921 | — |
| AAGTTGATATTTGTTGTTAGATGA | 922 | — |
| TGATGTGATAATGAGAATAAAGAA | 923 | 79 |
| AAAGTTTAGTTTGTATTAGTAGAG | 924 | — |
| AGTTTGATGTGATAGTAAATAGAA | 925 | — |
| AAGTGTTATTGAATGTGATGTTAT | 926 | — |
| AAATTGAAGTGTGATAATGTTTGT | 927 | — |
| GTTTAGTGATTAAAGATAGATTAG | 928 | 82 |
| ATAAGTGTATAAGAGAAGTGTTAA | 929 | — |
| ATGAATTTGTTTGTGATGAAGTTA | 930 | — |
| AAAGAATTGAGAAATGAAAGTTAG | 931 | — |
| AGTGTAAGAGTATAAAGTATTTGA | 932 | — |
| GAATTAAGATTGTTATATGTGAGT | 933 | — |
| TATGAAAGTGTTGTTTAAGTAAGA | 934 | — |
| TAAAGTAAATGTTATGTGAGAGAA | 935 | — |
| AAAGATATTGATTGAGATAGAGTT | 936 | — |
| AAGTGATATGAATATGTGAGAAAT | 937 | — |
| AAATAGAGTTTGTTAATGTAAGTG | 938 | — |
| GATTTAGATGAGTTAAGAATTTAG | 939 | — |
| TTGTAAATGAGTGTGAATATTGTA | 940 | — |
| AGTAGTGTATTTGAGATAATAGAA | 941 | — |
| TGAGTTAAAGAGTTGTTGATATTT | 942 | — |
| AAAGAGTGTATTAGAAATAGTTTG | 943 | — |
| GTTTAGTTATTTGATGAGATAATG | 944 | — |
| AAGTGTAAATGAATAAAGAGTTGT | 945 | — |
| AATAAAGTGAGTAGAAGTGTAATT | 946 | — |
| TATTGAGTTTGTGTAAAGAAGATA | 947 | — |
| TTTATAGTTGTTGTGTTGAAAGTT | 948 | — |
| ATGAAATATGATTGTGTTTGTTGT | 949 | — |
| AAAGAGATGTAAAGTGAGTTATTA | 950 | — |
| TTGAAGAAAGTTAGATGATGAATT | 951 | — |
| ATGTTATTTGTTTAGTTTGTGTGA | 952 | — |
| AAATATGAATTTGAAGAGAAGTGA | 953 | — |
| GATTAGATATAGAATATTGAAGAG | 954 | — |
| TTAGAATAAGAGAAATGTATGTGT | 955 | — |
| TTTATGAAAGAGAAGTGTATTATG | 956 | — |
| GTAAGTATTAAGTGTGATTTAGTA | 957 | — |
| ATAAAGAGAAGTAAAGAGTAAAGT | 958 | — |
| ATTGTTAATTGAAGTGTATGAAAG | 959 | — |
| TATATAGTTGAGTTGAGTAAGATT | 960 | — |
| TAGATGAGATATATGAAAGATAGT | 961 | — |
| ATAAGAAGATGATTTGTGTAAATG | 962 | — |
| TTAGTAATAAGAAAGATGAAGAGA | 963 | — |
| GATTTGTGAGTAAAGTAAATAGAA | 964 | — |
| AAATAGATGTAGAATTTGTGTGTT | 965 | — |
| GAAATTAGTGTTTGTGTGTATTAT | 966 | — |
| ATTTGAGTATGATAGAAGATTGTT | 967 | — |
| ATAGAGTTGAAGTATGTAAAGTTT | 968 | — |
| TAATTTGTGAATGTTGTTATTGTG | 969 | — |
| TTAGTTTATGAGAGTGAGATTTAA | 970 | — |
| GTTGTTAGAGTGTTTATGAAATTT | 971 | — |
| TTTATTGTGATGTGAAATAAGAGA | 972 | — |
| GTAAGTAATATGATAGTGATTAAG | 973 | — |
| TGAGATGATGTATATGTAGTAATA | 974 | — |
| AATTGAGAAAGAGATAAATGATAG | 975 | 85 |
| TTTGAAGTGATGTTAGAATGTTTA | 976 | — |
| AGTTGTTGTGTAATTGTTAGTAAA | 977 | — |
| ATAGTGAGAAGTGATAAGATATTT | 978 | — |
| GTGTGATAAGTAATTGAGTTAAAT | 979 | — |
| TAGTTATTGTTTGTGAATTTGAGA | 980 | — |
| ATAGTTGAATAGTAATTTGAAGAG | 981 | — |
| ATGTTTGTGTTTGAATAGAGAATA | 982 | — |
| TGATAAAGATATGAGAGATTGTAA | 983 | — |
| TAAAGATGAGATGTTGTTAAAGTT | 984 | — |
| AAGTGAAATTTGTAAGAATTAGTG | 985 | — |
| GAAATGAGAGTTATTGATAGTTTA | 986 | — |
| TTTGTAAATGAGATATAGTGTTAG | 987 | — |
| GTTAATTGTGATATTTGATTAGTG | 988 | — |
| AGAGTGTTGATAAAGATGTTTATA | 989 | — |
| AATTGTGAGAAATTGATAAGAAGA | 990 | — |
| TTAAAGAGAATTGAGAAGAGAAAT | 991 | — |
| TTGTTAGAAGAATTGAATGTATGT | 992 | — |
| AGTTAAGATATGTGTGATGTTTAA | 993 | — |
| TGAGTTATGTTGTAATAGAAATTG | 994 | — |
| TTAGATAAGTTTAGAGATTGAGAA | 995 | — |
| ATGAGTAATAAGAGTATTTGAAGT | 996 | — |
| TGTTTAAGTGTAATGATTTGTTAG | 997 | — |
| TTGAAGAAGATTGTTATTGTTGAA | 998 | — |
| TATAGAAAGATTAAAGAGTGAATG | 999 | — |
| TAAATTGTTAGAAATTTGAGTGTG | 1000 | — |
| ATTGTTAGTGTGTTATTGATTATG | 1001 | — |
| GAGAATTATGTGTGAATATAGAAA | 1002 | — |
| TTGATTGATAAAGTAAAGAGTGTA | 1003 | — |
| GTGTGTAAATTGAATATGTTAATG | 1004 | — |
| AAAGTAAAGAAAGAAGTTTGAAAG | 1005 | — |
| TTTAGTTGAAGAATAGAAAGAAAG | 1006 | — |
| GTGTAATAAGAGTGAATAGTAATT | 1007 | — |
| TATTGAAATAAGAGAGATTTGTGA | 1008 | — |
| ATGAGAAAGAAGAAGTTAAGATTT | 1009 | — |
| AAGAGTGAGTATATTGTTAAAGAA | 1010 | — |
| TTTGTAAAGTGATGATGTAAGATA | 1011 | — |
| GATGTTATGTGATGAAATATGTAT | 1012 | — |
| GTAGAATAAAGTGTTAAAGTGTTA | 1013 | — |
| AAAGAGTATGTGTGTATGATATTT | 1014 | — |
| AAAGATAAGAGTTAGTAAATTGTG | 1015 | — |
| AAGAATTAGAGAATAAGTGTGATA | 1016 | — |
| GATAAGAAAGTGAAATGTAAATTG | 1017 | 86 |
| GATGAAAGATGTTTAAAGTTTGTT | 1018 | — |
| AGTGTAAGTAATAAGTTTGAGAAA | 1019 | — |
| GTTGAGAATTAGAATTTGATAAAG | 1020 | 87 |
| TTAAGAAATTTGTATGTGTTGTTG | 1021 | — |
| AGAAGATTTAGATGAAATGAGTTT | 1022 | — |
| TAAGTTTGAGATAAAGATGATATG | 1023 | — |
| TGAGATAGTTTGTAATATGTTTGT | 1024 | — |
| AGTTTGAAATTGTAAGTTTGATGA | 1025 | — |
| TAGAATTGATTAATGATGAGTAGT | 1026 | — |
| AGAGATTTGTAATAAGTATTGAAG | 1027 | — |
| ATAATGATGTAATGTAAGTAGTGT | 1028 | — |
| TGAAATTTGATGAGAGATATGTTA | 1029 | — |
| TGTGTAAAGTATAGTTTATGTTAG | 1030 | — |
| TGAATAAGTGAAATAGAATGAATG | 1031 | — |
| AAAGAAAGATTGTAATAAGTAGAG | 1032 | — |
| AATGAAATAGTGTTAAATGAGTGT | 1033 | 89 |
| GTAGATAAAGATGTGAATTATGAT | 1034 | — |
| GATAGTATATGTGTGTATTTGTTT | 1035 | — |
| ATGTTTGTAGAAATGTTTGAAGAT | 1036 | — |
| AAATTTGTAGAGAGAAATTTGTTG | 1037 | — |
| TAGAATAAGATTAGTAAGTGTAGA | 1038 | — |
| TGATTTAGAGAAATATGAGTAGAA | 1039 | — |
| AATAGAGTATGTTGTTTATGAGAA | 1040 | — |
| GATGATGAAGAGTTTATTGTAAAT | 1041 | — |
| AAGTAAAGAAGAAGAAATGTGTTA | 1042 | — |
| TTGAAGAATTAAGTGTTTAGTGTA | 1043 | — |
| AGAAAGAATGTTGATTTATGATGT | 1044 | — |
| GATTAAAGAGATGTTGATTGAAAT | 1045 | — |
| AATGATAATTGTTGAGAGAGTAAT | 1046 | — |
| GTTTGTTGAAAGTGTAAAGTATAT | 1047 | 90 |
| TGAGTTATATGAGAAAGTGTAATT | 1048 | — |
| TTGTGAGAAAGAAGTATATAGAAT | 1049 | — |
| GTAAGTTTAGAGTTATAGAGTTTA | 1050 | — |
| GATAGATAGATAAGTTAATTGAAG | 1051 | — |
| AGAGATGATTGTTTATGTATTATG | 1052 | — |
| AAAGTTAAGAAATTGTAGTGATAG | 1053 | — |
| TTTGATATTGTTTGTGAGTGTATA | 1054 | — |
| ATTTGTAGAAAGTTGTTATGAGTT | 1055 | — |
| GATTTGAGTAAGTTTATAGATGAA | 1056 | — |
| AAGATAAAGTGAGTTGATTTAGAT | 1057 | — |
| GATATTGTAAGATATGTTGTAAAG | 1058 | — |
| GTAAGAGTGTATTGTAAGTTAATT | 1059 | — |
| GTGTGATTAGTAATGAAGTATTTA | 1060 | 91 |
| GTAAGAAAGATTAAGTGTTAGTAA | 1061 | — |
| AGTAGAAAGTTGAAATTGATTATG | 1062 | 92 |
| TAAGAGAAGTTGAGTAATGTATTT | 1063 | — |
| GTTAAGAAATAGTAGATAAGTGAA | 1064 | — |
| TAAGTAAATTGAAAGTGTATAGTG | 1065 | — |
| AAGATGTATGTTTATTGTTGTGTA | 1066 | — |
| ATTTAGAATATAGTGAAGAGATAG | 1067 | — |
| GTTATGAAAGAGTATGTGTTAAAT | 1068 | 93 |
| TATTATGTGAAGAAGAATGATTAG | 1069 | — |
| TAATAAGTTGAAGAGAATTGTTGT | 1070 | — |
| TGATGTTTGATGTAATTGTTAAAG | 1071 | — |
| GTGAAAGATTTGAGTTTGTATAAT | 1072 | — |
| AGAGAATATAGATTGAGATTTGTT | 1073 | — |
| TTTGAGATGTGATGATAAAGTTAA | 1074 | — |
| GTTGTAAATTGTAGTAAAGAAGTA | 1075 | 94 |
| GTGTTATGATGTTGTTTGTATTAT | 1076 | — |
| ATTATTGTGTAGATGTATTAAGAG | 1077 | — |
| GTTAGAAAGATTTAGAAGTTAGTT | 1078 | — |
| TTGTGTATTAAGAGAGTGAAATAT | 1079 | — |
| GTTTAAGATAGAAAGAGTGATTTA | 1080 | — |
| AATGAGAAATAGATAGTTATTGTG | 1081 | — |
| TGAATTGAATAAGAATTTGTTGTG | 1082 | 95 |
| AATAAGATTGAATTAGTGAGTAAG | 1083 | — |
| AATGTTTGAGAGATTTAGTAAAGA | 1084 | — |
| AGTTTAGAATAGAAATGTGTTTGA | 1085 | — |
| TATAAGTAAGTGTTAAGATTTGAG | 1086 | — |
| GTAGTGAATAAGTTAGTGTTAATA | 1087 | — |
| AAGTGTGTTAAAGTAAATGTAGAT | 1088 | — |
| AGAGATGTTTATGTTGTGAATTAA | 1089 | — |
| AGTTGAATATTGATGATAAGAAGA | 1090 | — |
| TGAATGTGAGATGTTTAGAATAAT | 1091 | — |
| AATAATGATGTAAGTTTGAGTTTG | 1092 | — |
| AAAGAGTGAATAGAAATAAGAGAA | 1093 | — |
| AATAAAGTTATTGAGAGAGTTTAG | 1094 | — |
| AGTAGTGTTGTAGTTTAGTATATA | 1095 | — |
| GTAAGAATGTATTAGATATTTGTG | 1096 | — |
| GATAAATGTTTGATAAAGTAGTTG | 1097 | — |
| ATAGTATGTATGTGTGAAGATTTA | 1098 | — |
| ATGAATGTAGAGTGATTAGTTTAA | 1099 | — |
| GTAGTATTTAGTGATGTAAGAATA | 1100 | — |
| AGAATTGTATTGAAGAAGAATATG | 1101 | — |
| TTTATAGAATTGAGAGAAGTTAAG | 1102 | — |
| AAAGTAGTAGAGATTTGAGAATTA | 1103 | — |
| TTTAAAGAAAGTATTGTAAGAGTG | 1104 | — |
| AAATTGAGAAAGTGAATGAAGTTT | 1105 | — |
| AAGAAATAAGTATGATAGTAGTAG | 1106 | — |
| ATTTGAATTGTATTGTAGTTTGTG | 1107 | — |
| AAGAGAATAATGTAGAGATATAAG | 1108 | — |
| TGTGTAATAGTTGTTAATGAGTAA | 1109 | — |
| TATAGTTGTAGTTTAGATGAATGT | 1110 | — |
| ATTGTGTTAGAATGATGTTAATAG | 1111 | — |
| GTTTGTATAGTATTTGATTGATGT | 1112 | — |
| AGAGTAAAGTATGAGTTATGAATA | 1113 | — |
| GAAAGTTTAAGTGATGTATATTGT | 1114 | 96 |
| TTAAATGATAAAGAGTAGTGAAGT | 1115 | — |
| TTAAATGTGTGAGAAGATGAATAA | 1116 | — |
| ATTTGTATAAAGTGAAGAAGAGAA | 1117 | 97 |
| TGATTAGTATTTGTGAAGAGATTT | 1118 | — |
| TTTGAATGAAATTGATGATAGATG | 1119 | — |
| AGAGTAAGATTAAGAATAAGAAAG | 1120 | — |
| ATTGAATTGAGAAGTGAAGTAAAT | 1121 | — |
| TTTAGAGAAGTATTGTTTGAAAGA | 1122 | — |
| TAAAGTGAAAGATTTGAAATGATG | 1123 | — |
| GAAAGTTAGAGAAATGTAGAAATT | 1124 | — |
| GTGAATAATGAAGAAGTTATGTTA | 1125 | 98 |
| TTGTGAATAAAGTAGATGTGTTAT | 1126 | — |
| TTATATGATATGAGTTTGTGTTGA | 1127 | — |
| TTGATTTGTGTGAGTATTAGTTAT | 1128 | — |
| AAAGTGATTAAGTTAGTTTGAGAT | 1129 | — |
| TTGTATTTGTATAATGTTGAAGAG | 1130 | — |
| GTTTGAAATTAGTGTGAGAAATAT | 1131 | — |
| AATGTTGAGATTGATAATGTTGAA | 1132 | — |
| TAGTAGTAGTATTGTTGTAATAAG | 1133 | — |
| GTTGTAATTTGAGTGTTAGTTATT | 1134 | — |
| TGAATATGATAGTTAGTAATTGTG | 1135 | — |
| TGATAGTATGTTTGTGATTAAAGA | 1136 | — |
| GATGTATAAAGAGTATGTTATAAG | 1137 | — |
| AGTGAGATTTAGAAGATGTTATTA | 1138 | — |
| ATGAGAATTTGTTAAAGAGAAAGT | 1139 | — |
| AAAGAATTAGTATGATAGATGAGA | 1140 | 99 |
| TAGAGTTGTATAGTTTATAGTTGA | 1141 | — |
| GTAGAATGATTGTTTAGAAGATTT | 1142 | — |
| GTTTATGTTTGAGAAGAGTTATTT | 1143 | — |
| TAGAAGTTTGAAAGTTATTGATTG | 1144 | — |
| GATGAAGAGTATTTGTTATATGTA | 1145 | — |
| GATGAATATAGTAAGTATTGAGTA | 1146 | 100 |
| TAGTGATGAAATTTGAGATAGATA | 1147 | — |
| GAAAGAAATTGAAGAGTTTGATAT | 1148 | — |
| ATTTGAGTATTTGTGTATTGAATG | 1149 | — |
| ATGAGTTGAAATTTGAAGTATTGT | 1150 | — |
| TTAATAGTGAGAGAGTATATGTAA | 1151 | — |
| ATTAAGAGAGTGAGTAAATGTAAA | 1152 | — |
| AAGAATAGATGAGATTAGAAATAG | 1153 | — |
| AGTTTAAAGAGTTAGAATTGAAAG | 1154 | — |
| GTAAGATTTGTTGAATAAAGAAGA | 1155 | — |
| AGAGAAAGAAGTTAAAGTGATATT | 1156 | — |
| TAATAGAGAAGAGATGTATGAATA | 1157 | — |
| TTATTAGTGATAAGTGAAGTTTAG | 1158 | — |
| ATAATGTAAAGATGAGTTTATGAG | 1159 | — |
| TTGATTTGAGAGTTGATAAGATTT | 1160 | — |
| ATGATTATTGTGTGTAGAATTAGA | 1161 | — |
| TATAAAGATATAGTAGATGATGTG | 1162 | — |
| TTTAGTTGAGATGAAGTTATTAGA | 1163 | — |
| ATTGAATTGATATAGTGTAAAGTG | 1164 | — |
| GAAGAAAGATTATTGTATTGAGTT | 1165 | — |
| ATTGAGTGTAGTGATTTAGAAATA | 1166 | — |
| AATAAAGTGTTTAAGAGTAGAGTA | 1167 | — |
| GTAGAGATAATTGATGTGTAATTT | 1168 | — |

1. A composition comprising a cleavage structure, said cleavage structure comprising:
a) a target nucleic acid having a first region, a second region and a third region, wherein said first region is located adjacent to and downstream from said second region and said second region is located to and downstream from said third region;
b) a first oligonucleotide having a 5′ portion and a 3′ portion, said 5′ portion of said first oligonucleotide having a sequence complementary to said second region of said target nucleic acid and said 3′ portion of said first oligonucleotide having a sequence complementary to said third region of said target nucleic acid; and
c) a second oligonucleotide having a 5′ portion, a central portion and a 3′ portion, said 5′ portion of said second oligonucleotide having a sequence complementary to said first region of said target nucleic acid, said central region of said second oligonucleotide having a sequence complementary to said second region of said target nucleic acid, and said 3′ portion of said second oligonucleotide having a sequence that is not base-paired to said target nucleic acid and is selected from a set of oligonucleotides based on a following group of sequences,

| | | | | | |
|---|---|---|---|---|---|
| 1 | 4 | 6 | 6 | 1 | 3 |
| 2 | 4 | 5 | 5 | 2 | 3 |
| 1 | 8 | 1 | 2 | 3 | 4 |
| 1 | 7 | 1 | 9 | 8 | 4 |
| 1 | 1 | 9 | 2 | 6 | 9 |
| 1 | 2 | 4 | 3 | 9 | 6 |
| 9 | 8 | 9 | 8 | 10 | 9 |
| 9 | 1 | 2 | 3 | 8 | 10 |
| 8 | 8 | 7 | 4 | 3 | 1 |
| 1 | 1 | 1 | 1 | 1 | 2 |
| 2 | 1 | 3 | 3 | 2 | 2 |
| 3 | 1 | 2 | 2 | 3 | 2 |
| 4 | 1 | 4 | 4 | 4 | 2 |
| 1 | 2 | 3 | 3 | 1 | 1 |
| 1 | 3 | 2 | 2 | 1 | 4 |
| 3 | 3 | 3 | 3 | 3 | 4 |
| 4 | 3 | 1 | 1 | 4 | 4 |
| 3 | 4 | 1 | 1 | 3 | 3 |
| 3 | 6 | 6 | 6 | 3 | 5 |
| 6 | 6 | 1 | 1 | 6 | 5 |
| 7 | 6 | 7 | 7 | 7 | 5 |
| 8 | 7 | 5 | 5 | 8 | 8 |
| 2 | 1 | 7 | 7 | 1 | 1 |
| 2 | 3 | 2 | 3 | 1 | 3 |
| 2 | 6 | 5 | 6 | 1 | 6 |
| 4 | 8 | 1 | 1 | 3 | 8 |
| 5 | 3 | 1 | 1 | 6 | 3 |
| 5 | 6 | 8 | 8 | 6 | 6 |
| 8 | 3 | 6 | 5 | 7 | 3 |
| 1 | 2 | 3 | 1 | 4 | 6 |
| 1 | 5 | 7 | 5 | 4 | 3 |
| 2 | 1 | 6 | 7 | 3 | 6 |
| 2 | 6 | 1 | 3 | 3 | 1 |
| 2 | 7 | 6 | 8 | 3 | 1 |
| 3 | 4 | 3 | 1 | 2 | 5 |
| 3 | 5 | 6 | 1 | 2 | 7 |
| 3 | 6 | 1 | 7 | 2 | 7 |
| 4 | 6 | 3 | 5 | 1 | 7 |
| 5 | 4 | 6 | 3 | 8 | 6 |
| 6 | 8 | 2 | 3 | 7 | 1 |
| 7 | 1 | 7 | 8 | 6 | 3 |
| 7 | 3 | 4 | 1 | 6 | 8 |
| 4 | 7 | 7 | 1 | 2 | 4 |
| 3 | 6 | 5 | 2 | 6 | 3 |
| 1 | 4 | 1 | 4 | 6 | 1 |
| 3 | 3 | 1 | 4 | 8 | 1 |
| 8 | 3 | 3 | 5 | 3 | 8 |
| 1 | 3 | 6 | 6 | 3 | 7 |
| 7 | 3 | 8 | 6 | 4 | 7 |
| 3 | 1 | 3 | 7 | 8 | 6 |
| 10 | 9 | 5 | 5 | 10 | 10 |
| 7 | 10 | 10 | 10 | 7 | 9 |
| 9 | 9 | 7 | 7 | 10 | 9 |
| 9 | 3 | 10 | 3 | 10 | 3 |
| 9 | 6 | 3 | 4 | 10 | 6 |
| 10 | 4 | 10 | 3 | 9 | 4 |
| 3 | 9 | 3 | 10 | 4 | 9 |
| 9 | 10 | 5 | 9 | 4 | 8 |
| 3 | 9 | 4 | 9 | 10 | 7 |
| 3 | 5 | 9 | 4 | 10 | 8 |
| 4 | 10 | 5 | 4 | 9 | 3 |
| 5 | 3 | 3 | 9 | 8 | 10 |
| 6 | 8 | 6 | 9 | 7 | 10 |
| 4 | 6 | 10 | 9 | 6 | 4 |
| 4 | 9 | 8 | 10 | 8 | 3 |
| 7 | 7 | 9 | 10 | 5 | 3 |
| 8 | 8 | 9 | 3 | 9 | 10 |
| 8 | 10 | 2 | 9 | 5 | 9 |
| 9 | 6 | 2 | 2 | 7 | 10 |
| 9 | 7 | 5 | 3 | 10 | 6 |
| 10 | 3 | 6 | 8 | 9 | 2 |
| 10 | 9 | 3 | 2 | 7 | 3 |
| 8 | 9 | 10 | 3 | 6 | 2 |
| 3 | 2 | 5 | 10 | 8 | 9 |
| 8 | 2 | 3 | 10 | 2 | 9 |
| 6 | 3 | 9 | 8 | 2 | 10 |
| 3 | 7 | 3 | 9 | 9 | 10 |
| 9 | 10 | 1 | 1 | 9 | 4 |
| 10 | 1 | 9 | 1 | 4 | 1 |
| 7 | 1 | 10 | 9 | 8 | 1 |
| 9 | 1 | 10 | 1 | 10 | 6 |
| 9 | 6 | 9 | 1 | 3 | 10 |
| 3 | 10 | 8 | 8 | 9 | 1 |
| 3 | 8 | 1 | 9 | 10 | 3 |
| 9 | 10 | 1 | 3 | 6 | 9 |
| 1 | 9 | 1 | 10 | 3 | 1 |
| 1 | 4 | 9 | 6 | 8 | 10 |
| 3 | 3 | 9 | 6 | 1 | 10 |
| 5 | 3 | 1 | 6 | 9 | 10 |
| 6 | 1 | 8 | 10 | 9 | 6 |
| 5 | 9 | 9 | 4 | 10 | 3 |
| 2 | 10 | 9 | 1 | 9 | 5 |
| 10 | 10 | 7 | 2 | 1 | 9 |
| 10 | 9 | 9 | 1 | 8 | 2 |
| 1 | 8 | 6 | 8 | 9 | 10 |
| 1 | 9 | 1 | 3 | 8 | 10 |
| 9 | 6 | 9 | 10 | 1 | 2 |
| 1 | 10 | 8 | 9 | 9 | 2 |
| 1 | 9 | 6 | 7 | 2 | 9 |
| 4 | 3 | 9 | 3 | 5 | 1 |
| 5 | 11 | 10 | 14 | 12 | 1 |
| 7 | 12 | 4 | 13 | 3 | 2 |
| 5 | 5 | 4 | 4 | 12 | 9 |
| 2 | 13 | 13 | 11 | 13 | 13 |
| 10 | 2 | 5 | 4 | 12 | 7 |
| 11 | 7 | 4 | 11 | 6 | 4 |
| 12 | 12 | 1 | 9 | 11 | 11 |
| 12 | 9 | 4 | 14 | 12 | 6 |
| 12 | 7 | 13 | 2 | 9 | 11 |
| 9 | 11 | 3 | 4 | 1 | 3 |
| 10 | 5 | 12 | 11 | 4 | 4 |
| 4 | 13 | 7 | 12 | 1 | 5 |
| 9 | 13 | 10 | 11 | 11 | 6 |
| 10 | 14 | 14 | 10 | 1 | 3 |
| 2 | 14 | 1 | 10 | 4 | 5 |
| 10 | 12 | 12 | 7 | 11 | 10 |
| 9 | 11 | 2 | 12 | 8 | 11 |
| 2 | 8 | 5 | 2 | 12 | 14 |
| 1 | 8 | 13 | 3 | 7 | 8 |
| 9 | 4 | 7 | 5 | 4 | 2 |
| 13 | 2 | 12 | 7 | 1 | 12 |
| 11 | 10 | 9 | 7 | 5 | 11 |
| 8 | 12 | 2 | 2 | 12 | 7 |
| 5 | 2 | 14 | 3 | 4 | 13 |
| 1 | 8 | 8 | 1 | 5 | 9 |
| 14 | 5 | 11 | 10 | 13 | 3 |
| 14 | 1 | 4 | 13 | 2 | 4 |
| 4 | 4 | 5 | 11 | 3 | 10 |
| 10 | 9 | 2 | 3 | 3 | 11 |
| 11 | 4 | 8 | 14 | 3 | 4 |
| 5 | 1 | 14 | 8 | 11 | 2 |
| 14 | 3 | 11 | 6 | 12 | 5 |
| 13 | 4 | 4 | 1 | 10 | 1 |
| 6 | 10 | 11 | 6 | 5 | 1 |
| 5 | 8 | 12 | 5 | 1 | 7 |
| 4 | 5 | 9 | 6 | 9 | 2 |
| 13 | 2 | 4 | 4 | 2 | 3 |
| 11 | 2 | 2 | 5 | 9 | 3 |
| 8 | 1 | 10 | 12 | 2 | 8 |
| 12 | 7 | 9 | 11 | 4 | 1 |
| 12 | 1 | 4 | 14 | 3 | 13 |
| 11 | 2 | 7 | 10 | 4 | 1 |
| 3 | 4 | 12 | 11 | 11 | 11 |
| 3 | 3 | 4 | 2 | 12 | 11 |
| 1 | 5 | 9 | 4 | 2 | 1 |
| 6 | 1 | 12 | 2 | 10 | 5 |
| 10 | 5 | 1 | 12 | 2 | 14 |
| 2 | 11 | 7 | 9 | 4 | 11 |
| 7 | 4 | 4 | 5 | 14 | 12 |
| 12 | 5 | 2 | 1 | 10 | 12 |
| 5 | 9 | 2 | 11 | 6 | 1 |
| 12 | 14 | 3 | 6 | 1 | 14 |
| 5 | 9 | 11 | 10 | 1 | 4 |
| 2 | 5 | 12 | 14 | 10 | 10 |
| 4 | 5 | 8 | 4 | 5 | 6 |
| 10 | 12 | 4 | 6 | 12 | 5 |
| 4 | 2 | 1 | 13 | 6 | 8 |
| 9 | 10 | 10 | 14 | 5 | 3 |
| 6 | 14 | 10 | 11 | 3 | 3 |
| 2 | 9 | 10 | 12 | 5 | 7 |
| 13 | 3 | 7 | 10 | 5 | 12 |
| 6 | 4 | 1 | 2 | 5 | 13 |
| 6 | 1 | 13 | 4 | 14 | 13 |
| 2 | 12 | 1 | 14 | 1 | 9 |
| 4 | 11 | 13 | 2 | 6 | 10 |
| 1 | 10 | 7 | 4 | 5 | 8 |
| 7 | 2 | 2 | 10 | 13 | 4 |
| 8 | 2 | 11 | 4 | 6 | 14 |
| 4 | 8 | 2 | 6 | 2 | 3 |
| 7 | 1 | 12 | 11 | 2 | 9 |
| 5 | 6 | 10 | 4 | 13 | 4 |
| 5 | 10 | 4 | 11 | 9 | 3 |
| 3 | 11 | 9 | 3 | 2 | 3 |
| 8 | 15 | 6 | 20 | 17 | 19 |
| 21 | 10 | 15 | 3 | 7 | 11 |
| 11 | 7 | 17 | 20 | 14 | 9 |
| 16 | 6 | 17 | 13 | 21 | 21 |
| 10 | 15 | 22 | 6 | 17 | 21 |
| 15 | 7 | 17 | 10 | 22 | 22 |
| 3 | 20 | 8 | 15 | 20 | 16 |
| 17 | 21 | 10 | 16 | 6 | 22 |
| 6 | 21 | 14 | 14 | 14 | 16 |
| 7 | 17 | 3 | 20 | 10 | 7 |
| 16 | 19 | 14 | 17 | 7 | 21 |
| 20 | 16 | 7 | 15 | 22 | 10 |
| 20 | 10 | 18 | 11 | 22 | 18 |
| 18 | 7 | 19 | 15 | 7 | 22 |
| 21 | 18 | 7 | 21 | 16 | 3 |
| 14 | 13 | 7 | 22 | 17 | 13 |
| 19 | 7 | 8 | 12 | 10 | 17 |
| 15 | 3 | 21 | 14 | 9 | 7 |
| 19 | 6 | 15 | 7 | 14 | 14 |
| 4 | 17 | 10 | 15 | 20 | 19 |
| 21 | 6 | 18 | 4 | 20 | 16 |
| 2 | 19 | 8 | 17 | 6 | 13 |
| 12 | 12 | 6 | 17 | 4 | 20 |
| 16 | 21 | 12 | 10 | 19 | 16 |
| 14 | 14 | 15 | 2 | 7 | 21 |
| 8 | 16 | 21 | 6 | 22 | 16 |
| 14 | 17 | 22 | 14 | 17 | 20 |
| 10 | 21 | 7 | 15 | 21 | 18 |
| 16 | 13 | 20 | 18 | 21 | 12 |
| 15 | 7 | 4 | 22 | 14 | 13 |
| 7 | 19 | 14 | 8 | 15 | 4 |
| 4 | 5 | 3 | 20 | 7 | 16 |
| 22 | 18 | 6 | 18 | 13 | 20 |
| 19 | 6 | 16 | 3 | 13 | 3 |
| 18 | 6 | 22 | 7 | 20 | 18 |
| 10 | 17 | 11 | 21 | 8 | 13 |
| 7 | 10 | 17 | 19 | 10 | 14 |

wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is abase in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which itis generated; and
(iii) the phantom sequence is not greater than or equal to ( 11/12×L) in length;
where L=L₁, or if L₁≠L₂, where L is the greater of L₁ and L₂; and
wherein any base present may be substituted by an analogue thereof.

2. The composition of claim 1, wherein one or more of said first and second oligonucleotides contain a 3′-terminal dideoxynucleotide.

3. The composition of claim 1, wherein the composition includes a plurality of said target nucleic acid sequences and a plurality of second oligonucleotide molecules such that each of said second oligonucleotide molecules has a distinct 3′ region.

4. The composition of claim 3, wherein the composition includes at least ten said second oligonucleotide molecules or at least eleven said second oligonucleotide molecules, or at least twelve said second oligonucleotide molecules, or at least thirteen said second oligonucleotide molecules, or at least fourteen said second oligonucleotide molecules, or at least fifteen said second oligonucleotide molecules, or at least sixteen said second oligonucleotide molecules, or at least seventeen said second oligonucleotide molecules, or at least eighteen said second oligonucleotide molecules, or at least nineteen said second oligonucleotide molecules, or at least twenty said second oligonucleotide molecules, or at least twenty-one said second oligonucleotide molecules, or at least twenty-two said second oligonucleotide molecules, or at least twenty-three said second oligonucleotide molecules, or at least twenty-four said second oligonucleotide molecules, or at least twenty-five said second oligonucleotide molecules, or at least twenty-six said second oligonucleotide molecules, or at least twenty-seven said second oligonucleotide molecules, or at least twenty-eight said second oligonucleotide molecules, or at least twenty-nine said second oligonucleotide molecules, or at least thirty said second oligonucleotide molecules, or at least thirty-one said second oligonucleotide molecules, or at least thirty-two said second oligonucleotide molecules, or at least thirty-three said second oligonucleotide molecules, or at least thirty-four said second oligonucleotide molecules, or at least thirty-five said second oligonucleotide molecules, or at least thirty-six said second oligonucleotide molecules, or at least thirty-seven said second oligonucleotide molecules, or at least thirty-eight said second oligonucleotide molecules, or at least thirty-nine said second oligonucleotide molecules, or at least forty said second oligonucleotide molecules, or at least forty-one said second oligonucleotide molecules, or at least forty-two said second oligonucleotide molecules, or at least forty-three said second oligonucleotide molecules, or at least forty-four said second oligonucleotide molecules, or at least forty-five said second oligonucleotide molecules, or at least forty-six said second oligonucleotide molecules, or at least forty seven said second oligonucleotide molecules, or at least forty-eight said second oligonucleotide molecules, or at least forty-nine said second oligonucleotide molecules, or at least fifty said second oligonucleotide molecules, or at least sixty said second oligonucleotide molecules, or at least seventy said second oligonucleotide molecules, or at least eighty said second oligonucleotide molecules, or at least ninety said second oligonucleotide molecules, or at least one hundred said second oligonucleotide molecules, or at least one hundred and ten said second oligonucleotide molecules, or at least one hundred and twenty said second oligonucleotide molecules, or at least one hundred and thirty said second oligonucleotide molecules, or at least one hundred and forty said second oligonucleotide molecules, or at least one hundred and fifty said second oligonucleotide molecules, or at least one hundred and sixty said second oligonucleotide molecules, or at least one hundred and seventy said second oligonucleotide molecules, or at least one hundred and eighty said second oligonucleotide molecules, or at least one hundred and ninety said second oligonucleotide molecules, or at least two hundred said second oligonucleotide molecules.

5. A method of detecting the presence of a target nucleic acid molecule by detecting non-target cleavage products, the method comprising:
a) providing:
i) a cleavage means,
ii) a target nucleic acid, said target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5, portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said second oligonucleotide having a sequence that is not base-paired to said target nucleic acid and is selected from a set of oligonucleotides based one a following group of sequences;

| | | | | | |
|---|---|---|---|---|---|
| 1 | 4 | 6 | 6 | 1 | 3 |
| 2 | 4 | 5 | 5 | 2 | 3 |
| 1 | 8 | 1 | 2 | 3 | 4 |
| 1 | 7 | 1 | 9 | 8 | 4 |
| 1 | 1 | 9 | 2 | 6 | 9 |
| 1 | 2 | 4 | 3 | 9 | 6 |
| 9 | 8 | 9 | 8 | 10 | 9 |
| 9 | 1 | 2 | 3 | 8 | 10 |
| 8 | 8 | 7 | 4 | 3 | 1 |
| 1 | 1 | 1 | 1 | 1 | 2 |
| 2 | 1 | 3 | 3 | 2 | 2 |
| 3 | 1 | 2 | 2 | 3 | 2 |
| 4 | 1 | 4 | 4 | 4 | 2 |
| 1 | 2 | 3 | 3 | 1 | 1 |
| 1 | 3 | 2 | 2 | 1 | 4 |
| 3 | 3 | 3 | 3 | 3 | 4 |
| 4 | 3 | 1 | 1 | 4 | 4 |
| 3 | 4 | 1 | 1 | 3 | 3 |
| 3 | 6 | 6 | 6 | 3 | 5 |
| 6 | 6 | 1 | 1 | 6 | 5 |
| 7 | 6 | 7 | 7 | 7 | 5 |
| 8 | 7 | 5 | 5 | 8 | 8 |
| 2 | 1 | 7 | 7 | 1 | 1 |
| 2 | 3 | 2 | 3 | 1 | 3 |
| 2 | 6 | 5 | 6 | 1 | 6 |
| 4 | 8 | 1 | 1 | 3 | 8 |
| 5 | 3 | 1 | 1 | 6 | 3 |
| 5 | 6 | 8 | 8 | 6 | 6 |
| 8 | 3 | 6 | 5 | 7 | 3 |
| 1 | 2 | 3 | 1 | 4 | 6 |
| 1 | 5 | 7 | 5 | 4 | 3 |
| 2 | 1 | 6 | 7 | 3 | 6 |
| 2 | 6 | 1 | 3 | 3 | 1 |
| 2 | 7 | 6 | 8 | 3 | 1 |
| 3 | 4 | 3 | 1 | 2 | 5 |
| 3 | 5 | 6 | 1 | 2 | 7 |
| 3 | 6 | 1 | 7 | 2 | 7 |
| 4 | 6 | 3 | 5 | 1 | 7 |
| 5 | 4 | 6 | 3 | 8 | 6 |
| 6 | 8 | 2 | 3 | 7 | 1 |
| 7 | 1 | 7 | 8 | 6 | 3 |
| 7 | 3 | 4 | 1 | 6 | 8 |
| 4 | 7 | 7 | 1 | 2 | 4 |
| 3 | 6 | 5 | 2 | 6 | 3 |
| 1 | 4 | 1 | 4 | 6 | 1 |
| 3 | 3 | 1 | 4 | 8 | 1 |
| 8 | 3 | 3 | 5 | 3 | 8 |
| 1 | 3 | 6 | 6 | 3 | 7 |
| 7 | 3 | 8 | 6 | 4 | 7 |
| 3 | 1 | 3 | 7 | 8 | 6 |
| 10 | 9 | 5 | 5 | 10 | 10 |
| 7 | 10 | 10 | 10 | 7 | 9 |
| 9 | 9 | 7 | 7 | 10 | 9 |
| 9 | 3 | 10 | 3 | 10 | 3 |
| 9 | 6 | 3 | 4 | 10 | 6 |
| 10 | 4 | 10 | 3 | 9 | 4 |
| 3 | 9 | 3 | 10 | 4 | 9 |
| 9 | 10 | 5 | 9 | 4 | 8 |
| 3 | 9 | 4 | 9 | 10 | 7 |
| 3 | 5 | 9 | 4 | 10 | 8 |
| 4 | 10 | 5 | 4 | 9 | 3 |
| 5 | 3 | 3 | 9 | 8 | 10 |
| 6 | 8 | 6 | 9 | 7 | 10 |
| 4 | 6 | 10 | 9 | 6 | 4 |
| 4 | 9 | 8 | 10 | 8 | 3 |
| 7 | 7 | 9 | 10 | 5 | 3 |
| 8 | 8 | 9 | 3 | 9 | 10 |
| 8 | 10 | 2 | 9 | 5 | 9 |
| 9 | 6 | 2 | 2 | 7 | 10 |
| 9 | 7 | 5 | 3 | 10 | 6 |
| 10 | 3 | 6 | 8 | 9 | 2 |
| 10 | 9 | 3 | 2 | 7 | 3 |
| 8 | 9 | 10 | 3 | 6 | 2 |
| 3 | 2 | 5 | 10 | 8 | 9 |
| 8 | 2 | 3 | 10 | 2 | 9 |
| 6 | 3 | 9 | 8 | 2 | 10 |
| 3 | 7 | 3 | 9 | 9 | 10 |
| 9 | 10 | 1 | 1 | 9 | 4 |
| 10 | 1 | 9 | 1 | 4 | 1 |
| 7 | 1 | 10 | 9 | 8 | 1 |
| 9 | 1 | 10 | 1 | 10 | 6 |
| 9 | 6 | 9 | 1 | 3 | 10 |
| 3 | 10 | 8 | 8 | 9 | 1 |
| 3 | 8 | 1 | 9 | 10 | 3 |
| 9 | 10 | 1 | 3 | 6 | 9 |
| 1 | 9 | 1 | 10 | 3 | 1 |
| 1 | 4 | 9 | 6 | 8 | 10 |
| 3 | 3 | 9 | 6 | 1 | 10 |
| 5 | 3 | 1 | 6 | 9 | 10 |
| 6 | 1 | 8 | 10 | 9 | 6 |
| 5 | 9 | 9 | 4 | 10 | 3 |
| 2 | 10 | 9 | 1 | 9 | 5 |
| 10 | 10 | 7 | 2 | 1 | 9 |
| 10 | 9 | 9 | 1 | 8 | 2 |
| 1 | 8 | 6 | 8 | 9 | 10 |
| 1 | 9 | 1 | 3 | 8 | 10 |
| 9 | 6 | 9 | 10 | 1 | 2 |
| 1 | 10 | 8 | 9 | 9 | 2 |
| 1 | 9 | 6 | 7 | 2 | 9 |
| 4 | 3 | 9 | 3 | 5 | 1 |
| 5 | 11 | 10 | 14 | 12 | 1 |
| 7 | 12 | 4 | 13 | 3 | 2 |
| 5 | 5 | 4 | 4 | 12 | 9 |
| 2 | 13 | 13 | 11 | 13 | 13 |
| 10 | 2 | 5 | 4 | 12 | 7 |
| 11 | 7 | 4 | 11 | 6 | 4 |
| 12 | 12 | 1 | 9 | 11 | 11 |
| 12 | 9 | 4 | 14 | 12 | 6 |
| 12 | 7 | 13 | 2 | 9 | 11 |
| 9 | 11 | 3 | 4 | 1 | 3 |
| 10 | 5 | 12 | 11 | 4 | 4 |
| 4 | 13 | 7 | 12 | 1 | 5 |
| 9 | 13 | 10 | 11 | 11 | 6 |
| 10 | 14 | 14 | 10 | 1 | 3 |
| 2 | 14 | 1 | 10 | 4 | 5 |
| 10 | 12 | 12 | 7 | 11 | 10 |
| 9 | 11 | 2 | 12 | 8 | 11 |
| 2 | 8 | 5 | 2 | 12 | 14 |
| 1 | 8 | 13 | 3 | 7 | 8 |
| 9 | 4 | 7 | 5 | 4 | 2 |
| 13 | 2 | 12 | 7 | 1 | 12 |
| 11 | 10 | 9 | 7 | 5 | 11 |
| 8 | 12 | 2 | 2 | 12 | 7 |
| 5 | 2 | 14 | 3 | 4 | 13 |
| 1 | 8 | 8 | 1 | 5 | 9 |
| 14 | 5 | 11 | 10 | 13 | 3 |
| 14 | 1 | 4 | 13 | 2 | 4 |
| 4 | 4 | 5 | 11 | 3 | 10 |
| 10 | 9 | 2 | 3 | 3 | 11 |
| 11 | 4 | 8 | 14 | 3 | 4 |
| 5 | 1 | 14 | 8 | 11 | 2 |
| 14 | 3 | 11 | 6 | 12 | 5 |
| 13 | 4 | 4 | 1 | 10 | 1 |
| 6 | 10 | 11 | 6 | 5 | 1 |
| 5 | 8 | 12 | 5 | 1 | 7 |
| 4 | 5 | 9 | 6 | 9 | 2 |
| 13 | 2 | 4 | 4 | 2 | 3 |
| 11 | 2 | 2 | 5 | 9 | 3 |
| 8 | 1 | 10 | 12 | 2 | 8 |
| 12 | 7 | 9 | 11 | 4 | 1 |
| 12 | 1 | 4 | 14 | 3 | 13 |
| 11 | 2 | 7 | 10 | 4 | 1 |
| 3 | 4 | 12 | 11 | 11 | 11 |
| 3 | 3 | 4 | 2 | 12 | 11 |
| 1 | 5 | 9 | 4 | 2 | 1 |
| 6 | 1 | 12 | 2 | 10 | 5 |
| 10 | 5 | 1 | 12 | 2 | 14 |
| 2 | 11 | 7 | 9 | 4 | 11 |
| 7 | 4 | 4 | 5 | 14 | 12 |
| 12 | 5 | 2 | 1 | 10 | 12 |
| 5 | 9 | 2 | 11 | 6 | 1 |
| 12 | 14 | 3 | 6 | 1 | 14 |
| 5 | 9 | 11 | 10 | 1 | 4 |
| 2 | 5 | 12 | 14 | 10 | 10 |
| 4 | 5 | 8 | 4 | 5 | 6 |
| 10 | 12 | 4 | 6 | 12 | 5 |
| 4 | 2 | 1 | 13 | 6 | 8 |
| 9 | 10 | 10 | 14 | 5 | 3 |
| 6 | 14 | 10 | 11 | 3 | 3 |
| 2 | 9 | 10 | 12 | 5 | 7 |
| 13 | 3 | 7 | 10 | 5 | 12 |
| 6 | 4 | 1 | 2 | 5 | 13 |
| 6 | 1 | 13 | 4 | 14 | 13 |
| 2 | 12 | 1 | 14 | 1 | 9 |
| 4 | 11 | 13 | 2 | 6 | 10 |
| 1 | 10 | 7 | 4 | 5 | 8 |
| 7 | 2 | 2 | 10 | 13 | 4 |
| 8 | 2 | 11 | 4 | 6 | 14 |
| 4 | 8 | 2 | 6 | 2 | 3 |
| 7 | 1 | 12 | 11 | 2 | 9 |
| 5 | 6 | 10 | 4 | 13 | 4 |
| 5 | 10 | 4 | 11 | 9 | 3 |
| 3 | 11 | 9 | 3 | 2 | 3 |
| 8 | 15 | 6 | 20 | 17 | 19 |
| 21 | 10 | 15 | 3 | 7 | 11 |
| 11 | 7 | 17 | 20 | 14 | 9 |
| 16 | 6 | 17 | 13 | 21 | 21 |
| 10 | 15 | 22 | 6 | 17 | 21 |
| 15 | 7 | 17 | 10 | 22 | 22 |
| 3 | 20 | 8 | 15 | 20 | 16 |
| 17 | 21 | 10 | 16 | 6 | 22 |
| 6 | 21 | 14 | 14 | 14 | 16 |
| 7 | 17 | 3 | 20 | 10 | 7 |
| 16 | 19 | 14 | 17 | 7 | 21 |
| 20 | 16 | 7 | 15 | 22 | 10 |
| 20 | 10 | 18 | 11 | 22 | 18 |
| 18 | 7 | 19 | 15 | 7 | 22 |
| 21 | 18 | 7 | 21 | 16 | 3 |
| 14 | 13 | 7 | 22 | 17 | 13 |
| 19 | 7 | 8 | 12 | 10 | 17 |
| 15 | 3 | 21 | 14 | 9 | 7 |
| 19 | 6 | 15 | 7 | 14 | 14 |
| 4 | 17 | 10 | 15 | 20 | 19 |
| 21 | 6 | 18 | 4 | 20 | 16 |
| 2 | 19 | 8 | 17 | 6 | 13 |
| 12 | 12 | 6 | 17 | 4 | 20 |
| 16 | 21 | 12 | 10 | 19 | 16 |
| 14 | 14 | 15 | 2 | 7 | 21 |
| 8 | 16 | 21 | 6 | 22 | 16 |
| 14 | 17 | 22 | 14 | 17 | 20 |
| 10 | 21 | 7 | 15 | 21 | 18 |
| 16 | 13 | 20 | 18 | 21 | 12 |
| 15 | 7 | 4 | 22 | 14 | 13 |
| 7 | 19 | 14 | 8 | 15 | 4 |
| 4 | 5 | 3 | 20 | 7 | 16 |
| 22 | 18 | 6 | 18 | 13 | 20 |
| 19 | 6 | 16 | 3 | 13 | 3 |
| 18 | 6 | 22 | 7 | 20 | 18 |
| 10 | 17 | 11 | 21 | 8 | 13 |
| 7 | 10 | 17 | 19 | 10 | 14 |

wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any o f the sequences;
(E) all of the sequences of a said group of oligonucleotides are read 5′ to 31 or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−0.1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12×L) in length;
where L=L₁, or if L₁≠L₂, where L is the greater of L₁ and L2; and
wherein any base present may be substituted by an analogue thereof;
b) mixing said cleavage means, said target nucleic acid, said first and second oligonucleotides to create a reaction mixture under reaction conditions such that at least said 5′ portion of said first oligonucleotide is annealed to said target nucleic acid and wherein at least said 5′ and central portion of said second oligonucleotide is annealed to said target nucleic acid so as to create a cleavage structure and wherein the combined melting temperature of said complementary regions within said 5′ and 3′ portions of said first oligonucleotide when annealed to said target nucleic acid is greater than the melting temperature of said 5′ and central portion of said first oligonucleotide, and wherein cleavage of said cleavage structure occurs to generate non-target cleavage products; and
c) detecting said non-target cleavage products.

6. The method of claim 5, wherein said reaction temperature is between approximately 50 and 70 degrees centigrade.

7. The method of claim 5, wherein said target nucleic acid comprises single-stranded DNA.

8. The method of claim 5, wherein said target nucleic acid comprises double-stranded DNA and prior to step (c), said reaction mixture is treated such that said double-stranded DNA is rendered substantially single-stranded.

9. The method of claim 5, wherein said treatment to render said double-stranded DNA is rendered substantially single-stranded by increasing the temperature.

10. The method of claim 5, wherein said target nucleic acid comprises RNA and wherein said first and second oligonucleotides comprise DNA.

11. The method of claim 5, wherein said cleavage means comprises a thermostable 5′ nuclease.

12. The method of claim 11, wherein a portion of the amino acid sequence is homologous to a portion of the amino acid sequence of a thermostable DNA polymerase derived from a thermophilic organism.

13. The method of claim 12, wherein said organism is selected from the group consisting of Thermus aquaticus, Thermus flavus and Thermus thermophilus.

14. The method of claim 5, wherein said source of target nucleic acid comprises a sample containing genomic DNA.

15. The method of claim 5, wherein said reaction conditions comprise providing a source of divalent cations.

16. The method of claim 15, wherein said divalent cation is selected from the group comprising Mn²⁺ and Mg²⁺ ions.

17. The method of claim 5, wherein the method includes a plurality of said target nucleic acid sequences and a plurality of said second oligonucleotide molecules such that each of said second oligonucleotide molecules has a distinct 3′ region.

18. The method of claim 5, wherein the method includes at least ten said second oligonucleotide molecules or at least eleven said second oligonucleotide molecules, or at least twelve said second oligonucleotide molecules, or at least thirteen said second oligonucleotide molecules, or at least fourteen said second oligonucleotide molecules, or at least fifteen said second oligonucleotide molecules, or at least sixteen said second oligonucleotide molecules, or at least seventeen said second oligonucleotide molecules, or at least eighteen said second oligonucleotide molecules, or at least nineteen said second oligonucleotide molecules, or at least twenty said second oligonucleotide molecules, or at least twenty-one said second oligonucleotide molecules, or at least twenty-two said second oligonucleotide molecules, or at least twenty-three said second oligonucleotide molecules, or at least twenty-four said second oligonucleotide molecules, or at least twenty-five said second oligonucleotide molecules, or at least twenty-six said second oligonucleotide molecules, or at least twenty-seven said second oligonucleotide molecules, or at least twenty-eight said second oligonucleotide molecules, or at least twenty-nine said second oligonucleotide molecules, or at least thirty said second oligonucleotide molecules, or at least thirty-one said second oligonucleotide molecules, or at least thirty-two said second oligonucleotide molecules, or at least thirty-three said second oligonucleotide molecules, or at least thirty-four said second oligonucleotide molecules, or at least thirty-five said second oligonucleotide molecules, or at least thirty-six said second oligonucleotide molecules, or at least thirty-seven said second oligonucleotide molecules, or at least thirty-eight said second oligonucleotide molecules, or at least thirty-nine said second oligonucleotide molecules, or at least forty said second oligonucleotide molecules, or at least forty-one said second oligonucleotide molecules, or at least forty-two said second oligonucleotide molecules, or at least forty-three said second oligonucleotide molecules, or at least forty-four said second oligonucleotide molecules, or at least forty-five said second oligonucleotide molecules or at least forty-six said second oligonucleotide molecules, or at least forty-seven said second oligonucleotide molecules, or at least forty-eight said second oligonucleotide molecules, or at least forty-nine said second oligonucleotide molecules, or at least fifty said second oligonucleotide molecules, or at least sixty said second oligonucleotide molecules, or at least seventy said second oligonucleotide molecules, or at least eighty said second oligonucleotide molecules, or at least ninety said second oligonucleotide molecules, or at least one hundred said second oligonucleotide molecules, or at least one hundred and ten said second oligonucleotide molecules, or at least one hundred and twenty said second oligonucleotide molecules, or at least one hundred and thirty said second oligonucleotide molecules, or at least one hundred and forty said second oligonucleotide molecules, or at least one hundred and fifty said second oligonucleotide molecules, or at least one hundred and sixty said second oligonucleotide molecules, or at least one hundred and seventy said second oligonucleotide molecules, or at least one hundred and eighty said second oligonucleotide molecules, or at least one hundred and ninety said second oligonucleotide molecules, or at least two hundred said second oligonucleotide molecules.

19. The method of claim 5, wherein said 3′ portion of said second oligonucleotide incorporates fluorescent molecule, a radiolabelled nucleotide, digoxigenin, biotinylation and the like.

20. A method of analyzing a biological sample comprising a plurality of target nucleic acid molecules for the presence of a mutation or polymorphism at a locus of each target nucleic acid molecule, the method comprising:
a) providing:
i) a cleavage means,
ii) a plurality of target nucleic acid molecules, each of said target nucleic acid molecules having a first region, a second region and a third region, wherein said first region is located adjacent to and downstream from said second region, and said second region is located adjacent to and downstream from said third region;
iii) a plurality of first oligonucleotide molecules, each having a 5′ and a 3′ portion, said 5′ portion of said first oligonucleotide molecules having a sequence complementary to said second region of said target nucleic acid molecules and said 3′ portion of said first oligonucleotide molecules having a sequence complementary to said third region of said target nucleic acid molecules;
iv) a plurality of second oligonucleotide molecules, each having a 5′ portion, a central portion and a 3′ portion, said 5′ portion of said second oligonucleotide molecules having a sequence complementary to said first region of said target nucleic acid molecules, said central portion of said second oligonucleotide molecules having a sequence complimentary to said second region of said target nucleic acid molecules, and said 3′ portion of said second oligonucleotide molecules having a sequence that is not base-paired to said target nucleic acid and is selected from a set of oligonucleotides based on a following group of sequences,

| | | | | | |
|---|---|---|---|---|---|
| 1 | 4 | 6 | 6 | 1 | 3 |
| 2 | 4 | 5 | 5 | 2 | 3 |
| 1 | 8 | 1 | 2 | 3 | 4 |
| 1 | 7 | 1 | 9 | 8 | 4 |
| 1 | 1 | 9 | 2 | 6 | 9 |
| 1 | 2 | 4 | 3 | 9 | 6 |
| 9 | 8 | 9 | 8 | 10 | 9 |
| 9 | 1 | 2 | 3 | 8 | 10 |
| 8 | 8 | 7 | 4 | 3 | 1 |
| 1 | 1 | 1 | 1 | 1 | 2 |
| 2 | 1 | 3 | 3 | 2 | 2 |
| 3 | 1 | 2 | 2 | 3 | 2 |
| 4 | 1 | 4 | 4 | 4 | 2 |
| 1 | 2 | 3 | 3 | 1 | 1 |
| 1 | 3 | 2 | 2 | 1 | 4 |
| 3 | 3 | 3 | 3 | 3 | 4 |
| 4 | 3 | 1 | 1 | 4 | 4 |
| 3 | 4 | 1 | 1 | 3 | 3 |
| 3 | 6 | 6 | 6 | 3 | 5 |
| 6 | 6 | 1 | 1 | 6 | 5 |
| 7 | 6 | 7 | 7 | 7 | 5 |
| 8 | 7 | 5 | 5 | 8 | 8 |
| 2 | 1 | 7 | 7 | 1 | 1 |
| 2 | 3 | 2 | 3 | 1 | 3 |
| 2 | 6 | 5 | 6 | 1 | 6 |
| 4 | 8 | 1 | 1 | 3 | 8 |
| 5 | 3 | 1 | 1 | 6 | 3 |
| 5 | 6 | 8 | 8 | 6 | 6 |
| 8 | 3 | 6 | 5 | 7 | 3 |
| 1 | 2 | 3 | 1 | 4 | 6 |
| 1 | 5 | 7 | 5 | 4 | 3 |
| 2 | 1 | 6 | 7 | 3 | 6 |
| 2 | 6 | 1 | 3 | 3 | 1 |
| 2 | 7 | 6 | 8 | 3 | 1 |
| 3 | 4 | 3 | 1 | 2 | 5 |
| 3 | 5 | 6 | 1 | 2 | 7 |
| 3 | 6 | 1 | 7 | 2 | 7 |
| 4 | 6 | 3 | 5 | 1 | 7 |
| 5 | 4 | 6 | 3 | 8 | 6 |
| 6 | 8 | 2 | 3 | 7 | 1 |
| 7 | 1 | 7 | 8 | 6 | 3 |
| 7 | 3 | 4 | 1 | 6 | 8 |
| 4 | 7 | 7 | 1 | 2 | 4 |
| 3 | 6 | 5 | 2 | 6 | 3 |
| 1 | 4 | 1 | 4 | 6 | 1 |
| 3 | 3 | 1 | 4 | 8 | 1 |
| 8 | 3 | 3 | 5 | 3 | 8 |
| 1 | 3 | 6 | 6 | 3 | 7 |
| 7 | 3 | 8 | 6 | 4 | 7 |
| 3 | 1 | 3 | 7 | 8 | 6 |
| 10 | 9 | 5 | 5 | 10 | 10 |
| 7 | 10 | 10 | 10 | 7 | 9 |
| 9 | 9 | 7 | 7 | 10 | 9 |
| 9 | 3 | 10 | 3 | 10 | 3 |
| 9 | 6 | 3 | 4 | 10 | 6 |
| 10 | 4 | 10 | 3 | 9 | 4 |
| 3 | 9 | 3 | 10 | 4 | 9 |
| 9 | 10 | 5 | 9 | 4 | 8 |
| 3 | 9 | 4 | 9 | 10 | 7 |
| 3 | 5 | 9 | 4 | 10 | 8 |
| 4 | 10 | 5 | 4 | 9 | 3 |
| 5 | 3 | 3 | 9 | 8 | 10 |
| 6 | 8 | 6 | 9 | 7 | 10 |
| 4 | 6 | 10 | 9 | 6 | 4 |
| 4 | 9 | 8 | 10 | 8 | 3 |
| 7 | 7 | 9 | 10 | 5 | 3 |
| 8 | 8 | 9 | 3 | 9 | 10 |
| 8 | 10 | 2 | 9 | 5 | 9 |
| 9 | 6 | 2 | 2 | 7 | 10 |
| 9 | 7 | 5 | 3 | 10 | 6 |
| 10 | 3 | 6 | 8 | 9 | 2 |
| 10 | 9 | 3 | 2 | 7 | 3 |
| 8 | 9 | 10 | 3 | 6 | 2 |
| 3 | 2 | 5 | 10 | 8 | 9 |
| 8 | 2 | 3 | 10 | 2 | 9 |
| 6 | 3 | 9 | 8 | 2 | 10 |
| 3 | 7 | 3 | 9 | 9 | 10 |
| 9 | 10 | 1 | 1 | 9 | 4 |
| 10 | 1 | 9 | 1 | 4 | 1 |
| 7 | 1 | 10 | 9 | 8 | 1 |
| 9 | 1 | 10 | 1 | 10 | 6 |
| 9 | 6 | 9 | 1 | 3 | 10 |
| 3 | 10 | 8 | 8 | 9 | 1 |
| 3 | 8 | 1 | 9 | 10 | 3 |
| 9 | 10 | 1 | 3 | 6 | 9 |
| 1 | 9 | 1 | 10 | 3 | 1 |
| 1 | 4 | 9 | 6 | 8 | 10 |
| 3 | 3 | 9 | 6 | 1 | 10 |
| 5 | 3 | 1 | 6 | 9 | 10 |
| 6 | 1 | 8 | 10 | 9 | 6 |
| 5 | 9 | 9 | 4 | 10 | 3 |
| 2 | 10 | 9 | 1 | 9 | 5 |
| 10 | 10 | 7 | 2 | 1 | 9 |
| 10 | 9 | 9 | 1 | 8 | 2 |
| 1 | 8 | 6 | 8 | 9 | 10 |
| 1 | 9 | 1 | 3 | 8 | 10 |
| 9 | 6 | 9 | 10 | 1 | 2 |
| 1 | 10 | 8 | 9 | 9 | 2 |
| 1 | 9 | 6 | 7 | 2 | 9 |
| 4 | 3 | 9 | 3 | 5 | 1 |
| 5 | 11 | 10 | 14 | 12 | 1 |
| 7 | 12 | 4 | 13 | 3 | 2 |
| 5 | 5 | 4 | 4 | 12 | 9 |
| 2 | 13 | 13 | 11 | 13 | 13 |
| 10 | 2 | 5 | 4 | 12 | 7 |
| 11 | 7 | 4 | 11 | 6 | 4 |
| 12 | 12 | 1 | 9 | 11 | 11 |
| 12 | 9 | 4 | 14 | 12 | 6 |
| 12 | 7 | 13 | 2 | 9 | 11 |
| 9 | 11 | 3 | 4 | 1 | 3 |
| 10 | 5 | 12 | 11 | 4 | 4 |
| 4 | 13 | 7 | 12 | 1 | 5 |
| 9 | 13 | 10 | 11 | 11 | 6 |
| 10 | 14 | 14 | 10 | 1 | 3 |
| 2 | 14 | 1 | 10 | 4 | 5 |
| 10 | 12 | 12 | 7 | 11 | 10 |
| 9 | 11 | 2 | 12 | 8 | 11 |
| 2 | 8 | 5 | 2 | 12 | 14 |
| 1 | 8 | 13 | 3 | 7 | 8 |
| 9 | 4 | 7 | 5 | 4 | 2 |
| 13 | 2 | 12 | 7 | 1 | 12 |
| 11 | 10 | 9 | 7 | 5 | 11 |
| 8 | 12 | 2 | 2 | 12 | 7 |
| 5 | 2 | 14 | 3 | 4 | 13 |
| 1 | 8 | 8 | 1 | 5 | 9 |
| 14 | 5 | 11 | 10 | 13 | 3 |
| 14 | 1 | 4 | 13 | 2 | 4 |
| 4 | 4 | 5 | 11 | 3 | 10 |
| 10 | 9 | 2 | 3 | 3 | 11 |
| 11 | 4 | 8 | 14 | 3 | 4 |
| 5 | 1 | 14 | 8 | 11 | 2 |
| 14 | 3 | 11 | 6 | 12 | 5 |
| 13 | 4 | 4 | 1 | 10 | 1 |
| 6 | 10 | 11 | 6 | 5 | 1 |
| 5 | 8 | 12 | 5 | 1 | 7 |
| 4 | 5 | 9 | 6 | 9 | 2 |
| 13 | 2 | 4 | 4 | 2 | 3 |
| 11 | 2 | 2 | 5 | 9 | 3 |
| 8 | 1 | 10 | 12 | 2 | 8 |
| 12 | 7 | 9 | 11 | 4 | 1 |
| 12 | 1 | 4 | 14 | 3 | 13 |
| 11 | 2 | 7 | 10 | 4 | 1 |
| 3 | 4 | 12 | 11 | 11 | 11 |
| 3 | 3 | 4 | 2 | 12 | 11 |
| 1 | 5 | 9 | 4 | 2 | 1 |
| 6 | 1 | 12 | 2 | 10 | 5 |
| 10 | 5 | 1 | 12 | 2 | 14 |
| 2 | 11 | 7 | 9 | 4 | 11 |
| 7 | 4 | 4 | 5 | 14 | 12 |
| 12 | 5 | 2 | 1 | 10 | 12 |
| 5 | 9 | 2 | 11 | 6 | 1 |
| 12 | 14 | 3 | 6 | 1 | 14 |
| 5 | 9 | 11 | 10 | 1 | 4 |
| 2 | 5 | 12 | 14 | 10 | 10 |
| 4 | 5 | 8 | 4 | 5 | 6 |
| 10 | 12 | 4 | 6 | 12 | 5 |
| 4 | 2 | 1 | 13 | 6 | 8 |
| 9 | 10 | 10 | 14 | 5 | 3 |
| 6 | 14 | 10 | 11 | 3 | 3 |
| 2 | 9 | 10 | 12 | 5 | 7 |
| 13 | 3 | 7 | 10 | 5 | 12 |
| 6 | 4 | 1 | 2 | 5 | 13 |
| 6 | 1 | 13 | 4 | 14 | 13 |
| 2 | 12 | 1 | 14 | 1 | 9 |
| 4 | 11 | 13 | 2 | 6 | 10 |
| 1 | 10 | 7 | 4 | 5 | 8 |
| 7 | 2 | 2 | 10 | 13 | 4 |
| 8 | 2 | 11 | 4 | 6 | 14 |
| 4 | 8 | 2 | 6 | 2 | 3 |
| 7 | 1 | 12 | 11 | 2 | 9 |
| 5 | 6 | 10 | 4 | 13 | 4 |
| 5 | 10 | 4 | 11 | 9 | 3 |
| 3 | 11 | 9 | 3 | 2 | 3 |
| 8 | 15 | 6 | 20 | 17 | 19 |
| 21 | 10 | 15 | 3 | 7 | 11 |
| 11 | 7 | 17 | 20 | 14 | 9 |
| 16 | 6 | 17 | 13 | 21 | 21 |
| 10 | 15 | 22 | 6 | 17 | 21 |
| 15 | 7 | 17 | 10 | 22 | 22 |
| 3 | 20 | 8 | 15 | 20 | 16 |
| 17 | 21 | 10 | 16 | 6 | 22 |
| 6 | 21 | 14 | 14 | 14 | 16 |
| 7 | 17 | 3 | 20 | 10 | 7 |
| 16 | 19 | 14 | 17 | 7 | 21 |
| 20 | 16 | 7 | 15 | 22 | 10 |
| 20 | 10 | 18 | 11 | 22 | 18 |
| 18 | 7 | 19 | 15 | 7 | 22 |
| 21 | 18 | 7 | 21 | 16 | 3 |
| 14 | 13 | 7 | 22 | 17 | 13 |
| 19 | 7 | 8 | 12 | 10 | 17 |
| 15 | 3 | 21 | 14 | 9 | 7 |
| 19 | 6 | 15 | 7 | 14 | 14 |
| 4 | 17 | 10 | 15 | 20 | 19 |
| 21 | 6 | 18 | 4 | 20 | 16 |
| 2 | 19 | 8 | 17 | 6 | 13 |
| 12 | 12 | 6 | 17 | 4 | 20 |
| 16 | 21 | 12 | 10 | 19 | 16 |
| 14 | 14 | 15 | 2 | 7 | 21 |
| 8 | 16 | 21 | 6 | 22 | 16 |
| 14 | 17 | 22 | 14 | 17 | 20 |
| 10 | 21 | 7 | 15 | 21 | 18 |
| 16 | 13 | 20 | 18 | 21 | 12 |
| 15 | 7 | 4 | 22 | 14 | 13 |
| 7 | 19 | 14 | 8 | 15 | 4 |
| 4 | 5 | 3 | 20 | 7 | 16 |
| 22 | 18 | 6 | 18 | 13 | 20 |
| 19 | 6 | 16 | 3 | 13 | 3 |
| 18 | 6 | 22 | 7 | 20 | 18 |
| 10 | 17 | 11 | 21 | 8 | 13 |
| 7 | 10 | 17 | 19 | 10 | 14 |

wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(C) each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12×L) in length;
where L=L₁, or if L₁≠L₂, where L is the greater of L₁ and L₂; and
wherein any base present may be substituted by an analogue thereof;
b) mixing said cleavage means, said target nucleic acid, said first and second oligonucleotides to create a reaction mixture under reaction conditions such that at least said 5′ portion of said first oligonucleotide is annealed to said target nucleic acid and wherein at least said 5′ and central portion of said second oligonucleotide is annealed to said target nucleic acid so as to create a cleavage structure and wherein the combined melting temperature of said complementary regions within said 5′ and 3′ portions of said first oligonucleotide when annealed to said target nucleic acid is greater than the melting temperature of said 5′ and central portion of said first oligonucleotide, and wherein cleavage of said cleavage structure occurs to generate non-target cleavage products; and
c) detecting said non-target cleavage products.

21. The method of claim 20, wherein said reaction temperature is between approximately 50 and 70 degrees centigrade.

22. The method of claim 20, wherein said target nucleic acid molecules comprises single-stranded DNA.

23. The method of claim 20, wherein said target nucleic acid molecules comprises double-stranded DNA and prior to step (c), said reaction mixture is treated such that said double-stranded DNA is rendered substantially single-stranded.

24. The method of claim 20, wherein said treatment to render said double-stranded DNA is rendered substantially single-stranded by increasing the temperature.

25. The method of claim 20, wherein said target nucleic acid molecules comprises RNA and wherein said first and second oligonucleotide molecules comprise DNA.

26. The method of claim 20, wherein said cleavage means comprises a thermostable 5′ nuclease.

27. The method of claim 26, wherein a portion of the amino acid sequence is homologous to a portion of the amino acid sequence of a thermostable DNA polymerase derived from a thermophilic organism.

28. The method of claim 28, wherein said organism is selected from the group consisting of Thermus aquaticus, Thermus flavus and Thermus thermophilus.

29. The method of claim 20, wherein said source of target nucleic acid molecules comprises a sample containing genomic DNA.

30. The method of claim 20, wherein said reaction conditions comprise providing a source of divalent cations.

31. The method of claim 30, wherein said divalent cation is selected from the group comprising Mn²⁺ and Mg²⁺ ions.

32. The method of claim 20, wherein the method includes a plurality of said target nucleic acid sequences and a plurality of said second oligonucleotide molecules such that each of said second oligonucleotide molecules has a distinct 3′ region.

33. The method of claim 20, wherein the method includes at least ten said second oligonucleotide molecules or at least eleven said second oligonucleotide molecules, or at least twelve said second oligonucleotide molecules, or at least thirteen said second oligonucleotide molecules, or at least fourteen said second oligonucleotide molecules, or at least fifteen said second oligonucleotide molecules, or at least sixteen said second oligonucleotide molecules, or at least seventeen said second oligonucleotide molecules, or at least eighteen said second oligonucleotide molecules, or at least nineteen said second oligonucleotide molecules, or at least twenty said second oligonucleotide molecules, or at least twenty-one said second oligonucleotide molecules, or at least twenty-two said second oligonucleotide molecules, or at least twenty-three said second oligonucleotide molecules, or at least twenty-four said second oligonucleotide molecules, or at least twenty-five said second oligonucleotide molecules, or at least twenty-six said second oligonucleotide molecules, or at least twenty-seven said second, oligonucleotide molecules, or at least twenty-eight said second oligonucleotide molecules, or at least twenty-nine said second oligonucleotide molecules, or at least thirty said second oligonucleotide molecules, or at least thirty-one said second oligonucleotide molecules, or at least thirty-two said second oligonucleotide molecules, or at least thirty-three said second oligonucleotide molecules, or at least thirty-four said second oligonucleotide molecules, or at least thirty-five said second oligonucleotide molecules, or at least thirty-six said second oligonucleotide molecules, or at least thirty-seven said second oligonucleotide molecules, or at least thirty-eight said second oligonucleotide molecules, or at least thirty-nine said second oligonucleotide molecules, or at least forty said second oligonucleotide molecules, or at least forty-one said second oligonucleotide molecules, or at least forty-two said second oligonucleotide molecules, or at least forty-three said second oligonucleotide molecules, or at least forty-four said second oligonucleotide molecules, or at least forty-five said second oligonucleotide molecules, or at least forty-six said second oligonucleotide molecules, or at least forty-seven said second oligonucleotide molecules, or at least forty-eight said second oligonucleotide molecules, or at least forty-nine said second oligonucleotide molecules, or at least fifty said second oligonucleotide molecules, or at least sixty said second oligonucleotide molecules, or at least seventy said second oligonucleotide molecules, or at least eighty said second oligonucleotide molecules, or at least ninety said second oligonucleotide molecules, or at least one hundred said second oligonucleotide molecules, or at least one hundred and ten said second oligonucleotide molecules, or at least one hundred and twenty said second oligonucleotide molecules, or at least one hundred and thirty said second oligonucleotide molecules, or at least one hundred and forty said second oligonucleotide molecules, or at least one hundred and fifty said second oligonucleotide molecules, or at least one hundred and sixty said second oligonucleotide molecules, or at least one hundred and seventy said second oligonucleotide molecules, or at least one hundred and eighty said second oligonucleotide molecules, or at least one hundred and ninety said second oligonucleotide molecules, or at least two hundred said second oligonucleotide molecules.

34. The method of claim 20, wherein said 3′ portion of said second oligonucleotide molecules incorporates a fluorescent molecule, a radiolabelled nucleotide, digoxigenin, biotinylation and the like.

35. A composition comprising a cleavage structure, said cleavage structure comprising:
a) a target nucleic acid having a first region, a second region and a third region, wherein said first region is located adjacent to and downstream from said second region and said second region is located to and downstream from said third region;
b) a first oligonucleotide having a 5′ portion and a 3′ portion, said 5′ portion of said first oligonucleotide having a sequence complementary to said second region of said target nucleic acid and said 3′ portion of said first oligonucleotide having a sequence complementary to said third region of said target nucleic acid; and
c) a second oligonucleotide having a 5′ portion, a central portion and a 3′ portion, said 5′ portion of said second oligonucleotide having a sequence complementary to said first region of said target nucleic acid, said central region of said second oligonucleotide having a sequence complementary to said second region of said target nucleic acid, and said 3′ portion of said second oligonucleotide having a sequence that is not base-paired to said target nucleic acid and is selected from a set of oligonucleotides based on a following group of sequences:

| | | | | | | | | | | | | | | | | | | | | | | | |
|---|---|---|---|---|---|---|---|---|---|---|---|---|---|---|---|---|---|---|---|---|---|---|---|
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 3 | 2 | 2 | 2 | 1 | 2 | 2 | 3 | 2 | 1 | 1 | 1 | 3 | 1 | 2 | 3 | 2 | 1 | 1 | 2 | 3 | 3 | 1 | 1 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 2 | 3 | 2 | 3 | 2 | 1 | 2 | 1 | 1 | 3 | 2 | 1 | 1 | 2 | 2 | 2 | 3 | 2 | 1 | 1 | 3 | 1 | 1 | 3 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 3 | 2 | 3 | 2 | 2 | 1 | 2 | 2 | 1 | 3 | 2 | 2 | 3 | 1 | 1 | 3 | 2 | 2 | 1 | 3 | 1 | 3 | 2 | 1 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | 1 | 1 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

wherein each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3 S 20., M4 s 16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein
the score of an alignment is determined according to the equation (A×m)−(B×mm)−(C×(og+eg))−(D×eg)), wherein:
for each of (i) to (iv):
(i) m=6, mm=6, og=0 and eg=6,
(ii) m=6, mm=6, og=5 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv)
b) mixing said cleavage means, said target nucleic acid, said first and second oligonucleotides to create a reaction mixture under reaction conditions such that at least said 5′ portion of said first oligonucleotide is annealed to said target nucleic acid and wherein at least said 5′ and central portion of said second oligonucleotide is annealed to said target nucleic acid so as to create a cleavage structure and wherein the combined melting temperature of said complementary regions within said 5′ and 3′ portions of said first oligonucleotide when annealed to said target nucleic acid is greater than the melting temperature of said 5′ and central portion of said first oligonucleotide, and wherein cleavage of said cleavage structure occurs to generate non-target cleavage products; and
c) detecting said non-target cleavage products.

36. The composition of claim 35, wherein one or more of said first and second oligonucleotides contain a 3′-terminal dideoxynucleotide.

37. The composition of claim 35, wherein the composition includes a plurality of said target nucleic acid molecules and a plurality of said second oligonucleotide molecules such that each of said second oligonucleotide molecules has a distinct 3′ region.

38. The composition of claim 35, wherein the composition includes at least ten, or twenty, or thirty, or forty, or fifty, or sixty, or seventy, or eighty, or ninety, or one hundred, or one hundred and ten, or one hundred and twenty, or one hundred and thirty, or one hundred and forty, or one hundred and fity, or one hundred and sixty said second oligonucelotide molecules, or comprising one hundred and seventy said second oligonucleotide molecules, or comprising one hundred and eighty said second oligonucleotide molecules, or comprising one hundred and ninety said second oligonucleotide molecules, or comprising two hundred said second oligonucleotide molecules, or comprising two hundred and twenty said second oligonucleotide molecules, or comprising two hundred and forty said second oligonucleotide molecules, or comprising two hundred and sixty said second oligonucleotide molecules, or comprising two hundred and eighty said second oligonucleotide molecules, or comprising three hundred said second oligonucleotide molecules, or comprising four hundred said second oligonucleotide molecules, or comprising five hundred said second oligonucleotide molecules, or comprising six hundred said second oligonucleotide molecules, or comprising seven hundred said second oligonucleotide molecules, or comprising eight hundred said second oligonucleotide molecules, or comprising nine hundred said second oligonucleotide molecules, or comprising one thousand said second oligonucleotide molecules, or comprising eleven hundred said second oligonucleotide molecules.

39. A method of detecting the presence of a target nucleic acid molecule by detecting non-target cleavage products, the method comprising:
a) providing:
i) a cleavage means,
ii) a target nucleic acid, said target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5′ portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said second oligonucleotide having a sequence that is not base-paired to said target nucleic acid and is selected from a set of oligonucleotides based on a following group of sequences,

| | | | | | | | | | | | | | | | | | | | | | | | |
|---|---|---|---|---|---|---|---|---|---|---|---|---|---|---|---|---|---|---|---|---|---|---|---|
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | 1 | 1 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

wherein each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein:
the score of an alignment is determined according to the equation (A×m)−(B×mm)−(C×(og+eg))−(D×eg)), wherein:
for each of (i) to (iv):
(i) m=6, mm=6, og=0 and eg=6,
(ii) m=6, mm=6, og=5 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii)., (iii) and (iv).
b) mixing said cleavage means, said target nucleic acid, said first and second oligonucleotides to create a reaction mixture under reaction conditions such that at least said 5′ portion of said first oligonucleotide is annealed to said target nucleic acid and wherein at least said 5′ and central portion of said second oligonucleotide is annealed to said target nucleic acid so as to create a cleavage structure and wherein the combined melting temperature of said complementary regions within said 5′ and 3′ portions of said first oligonucleotide when annealed to said target nucleic acid is greater than the melting temperature of said 5′ and central portion of said first oligonucleotide, and wherein cleavage of said cleavage structure occurs to generate non-target cleavage products; and
c) detecting said non-target cleavage products.

40. The composition of claim 39, wherein one or more of said first and second oligonucleotides contain a 3′-terminal dideoxynucleotide.

41. The composition of claim 39, wherein the composition includes a plurality of said target nucleic acid sequences and a plurality of said second oligonucleotide molecules such that each of said second oligonucleotide molecules has a distinct 3′ region.

43. The composition of claim 39, wherein the composition includes at least ten, or twenty, or thirty, or forty, or fifty, or sixty, or seventy, or eighty, or ninety, or one hundred, or one hundred and ten, or one hundred and twenty, or one hundred and thirty, or one hundred and forty, or one hundred and fity, or one hundred and sixty said second oligonucelotide molecules, or comprising one hundred and seventy said second oligonucleotide molecules, or comprising one hundred and eighty said second oligonucleotide molecules, or comprising one hundred and ninety said second oligonucleotide molecules, or comprising two hundred said second oligonucleotide molecules, or comprising two hundred and twenty said second oligonucleotide molecules, or comprising two hundred and forty said second oligonucleotide molecules, or comprising two hundred and sixty said second oligonucleotide molecules, or comprising two hundred and eighty said second oligonucleotide molecules, or comprising three hundred said second oligonucleotide molecules, or comprising four hundred said second oligonucleotide molecules, or comprising five hundred said second oligonucleotide molecules, or comprising six hundred said second oligonucleotide molecules, or comprising seven hundred said second oligonucleotide molecules, or comprising eight hundred said second oligonucleotide molecules, or comprising nine hundred said second oligonucleotide molecules, or comprising one thousand said second oligonucleotide molecules, or comprising eleven hundred said second oligonucleotide molecules.

44. A method of analyzing a biological sample comprising a plurality of target nucleic acid molecules for the presence of a mutation or polymorphism at a locus of each target nucleic acid molecule, the method comprising:
a) providing:
i) a cleavage means,
ii) a plurality of target nucleic acid molecules, each of said target nucleic acid molecules having a first region, a second region and a third region, wherein said first region is located adjacent to and downstream from said second region, and said second region is located adjacent to and downstream from said third region;
iii) a plurality of first oligonucleotide molecules, each having a 5′ and a 3′ portion, said 5′ portion of said first oligonucleotide molecules having a sequence complementary to said second region of said target nucleic acid molecules and said 3′ portion of said first oligonucleotide molecules having a sequence complementary to said third region of said target nucleic acid molecules;
iv) a plurality of second oligonucleotide molecules, each having a 5′ portion, a central portion and a 3′ portion, said 5′ portion of said second oligonucleotide molecules having a sequence complementary to said first region of said target nucleic acid molecules, said central portion of said second oligonucleotide molecules having a sequence complimentary to said second region of said target nucleic acid molecules, and said 3′ portion of said second oligonucleotide molecules having a sequence that is not base-paired to said target nucleic acid and is selected from a set of oligonucleotides based on a following group of sequences,

| | | | | | | | | | | | | | | | | | | | | | | | |
|---|---|---|---|---|---|---|---|---|---|---|---|---|---|---|---|---|---|---|---|---|---|---|---|
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | 1 | 1 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

wherein each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein:
the score of an alignment is determined according to the equation (A×m)−(B×mm)−(C×(og+eg))−(D×eg)), wherein:
for each of (i) to (iv):
(i) m=6, mm=6, og=0 and eg=6,
(ii) m=6, mm=6, og=5 and eg=1,
(iii) m=6, mm=0.2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv).
b) mixing said cleavage means, said target nucleic acid, said first and second oligonucleotides to create a reaction mixture under reaction conditions such that at least said 5′ portion of said first oligonucleotide is annealed to said target nucleic acid and wherein at least said 5′ and central portion of said second oligonucleotide is annealed to said target nucleic acid so as to create a cleavage structure and wherein the combined melting temperature of said complementary regions within said 5′ and 3′ portions of said first oligonucleotide when annealed to said target nucleic acid is greater than the melting temperature of said 5′ and central portion of said first oligonucleotide, and wherein cleavage of said cleavage structure occurs to generate non-target cleavage products; and
c) detecting said non-target cleavage products.

45. The method of claim 44, wherein said reaction temperature is between approximately 50 and 70 degrees centigrade.

46. The method of claim 44; wherein said target nucleic acid molecules comprises single-stranded DNA.

47. The method of claim 44, wherein said target nucleic acid molecules comprises double-stranded DNA and prior to step (c), said reaction mixture is treated such that said double-stranded DNA is rendered substantially single-stranded.

48. The method of claim 47, wherein said treatment to render said double-stranded DNA is rendered substantially single-stranded by increasing the temperature.

49. The method of claim 44, wherein said target nucleic acid molecules comprises RNA and wherein said first and second oligonucleotide molecules comprise DNA.

50. The method of claim 44, wherein said cleavage means comprises a thermostable 5′ nuclease.

51. The method of claim 50, wherein a portion of the amino acid sequence is homologous to a portion of the amino acid sequence of a thermostable DNA polymerase derived from a thermophilic organism.

52. The method of claim 51, wherein said organism is selected from the group consisting of Thermus aquaticus, Thermus flavus and Thermus thermophilus.

53. The method of claim 44, wherein said source of target nucleic acid molecules comprises a sample containing genomic DNA.

54. The method of claim 44, wherein said reaction conditions comprise providing a source of divalent cations.

55. A method of detecting the presence of a target nucleic acid molecule by detecting non-target cleavage products, the method comprising:
a) providing:
i) a cleavage means,
ii) a first target nucleic acid, said first target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5′ portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said second oligonucleotide having a sequence that is not base-paired to said target nucleic acid and is selected from a set of oligonucleotides, based on a following group of sequences each having a 3′ and 5′ portion,

| | | | | | |
|---|---|---|---|---|---|
| 1 | 4 | 6 | 6 | 1 | 3 |
| 2 | 4 | 5 | 5 | 2 | 3 |
| 1 | 8 | 1 | 2 | 3 | 4 |
| 1 | 7 | 1 | 9 | 8 | 4 |
| 1 | 1 | 9 | 2 | 6 | 9 |
| 1 | 2 | 4 | 3 | 9 | 6 |
| 9 | 8 | 9 | 8 | 10 | 9 |
| 9 | 1 | 2 | 3 | 8 | 10 |
| 8 | 8 | 7 | 4 | 3 | 1 |
| 1 | 1 | 1 | 1 | 1 | 2 |
| 2 | 1 | 3 | 3 | 2 | 2 |
| 3 | 1 | 2 | 2 | 3 | 2 |
| 4 | 1 | 4 | 4 | 4 | 2 |
| 1 | 2 | 3 | 3 | 1 | 1 |
| 1 | 3 | 2 | 2 | 1 | 4 |
| 3 | 3 | 3 | 3 | 3 | 4 |
| 4 | 3 | 1 | 1 | 4 | 4 |
| 3 | 4 | 1 | 1 | 3 | 3 |
| 3 | 6 | 6 | 6 | 3 | 5 |
| 6 | 6 | 1 | 1 | 6 | 5 |
| 7 | 6 | 7 | 7 | 7 | 5 |
| 8 | 7 | 5 | 5 | 8 | 8 |
| 2 | 1 | 7 | 7 | 1 | 1 |
| 2 | 3 | 2 | 3 | 1 | 3 |
| 2 | 6 | 5 | 6 | 1 | 6 |
| 4 | 8 | 1 | 1 | 3 | 8 |
| 5 | 3 | 1 | 1 | 6 | 3 |
| 5 | 6 | 8 | 8 | 6 | 6 |
| 8 | 3 | 6 | 5 | 7 | 3 |
| 1 | 2 | 3 | 1 | 4 | 6 |
| 1 | 5 | 7 | 5 | 4 | 3 |
| 2 | 1 | 6 | 7 | 3 | 6 |
| 2 | 6 | 1 | 3 | 3 | 1 |
| 2 | 7 | 6 | 8 | 3 | 1 |
| 3 | 4 | 3 | 1 | 2 | 5 |
| 3 | 5 | 6 | 1 | 2 | 7 |
| 3 | 6 | 1 | 7 | 2 | 7 |
| 4 | 6 | 3 | 5 | 1 | 7 |
| 5 | 4 | 6 | 3 | 8 | 6 |
| 6 | 8 | 2 | 3 | 7 | 1 |
| 7 | 1 | 7 | 8 | 6 | 3 |
| 7 | 3 | 4 | 1 | 6 | 8 |
| 4 | 7 | 7 | 1 | 2 | 4 |
| 3 | 6 | 5 | 2 | 6 | 3 |
| 1 | 4 | 1 | 4 | 6 | 1 |
| 3 | 3 | 1 | 4 | 8 | 1 |
| 8 | 3 | 3 | 5 | 3 | 8 |
| 1 | 3 | 6 | 6 | 3 | 7 |
| 7 | 3 | 8 | 6 | 4 | 7 |
| 3 | 1 | 3 | 7 | 8 | 6 |
| 10 | 9 | 5 | 5 | 10 | 10 |
| 7 | 10 | 10 | 10 | 7 | 9 |
| 9 | 9 | 7 | 7 | 10 | 9 |
| 9 | 3 | 10 | 3 | 10 | 3 |
| 9 | 6 | 3 | 4 | 10 | 6 |
| 10 | 4 | 10 | 3 | 9 | 4 |
| 3 | 9 | 3 | 10 | 4 | 9 |
| 9 | 10 | 5 | 9 | 4 | 8 |
| 3 | 9 | 4 | 9 | 10 | 7 |
| 3 | 5 | 9 | 4 | 10 | 8 |
| 4 | 10 | 5 | 4 | 9 | 3 |
| 5 | 3 | 3 | 9 | 8 | 10 |
| 6 | 8 | 6 | 9 | 7 | 10 |
| 4 | 6 | 10 | 9 | 6 | 4 |
| 4 | 9 | 8 | 10 | 8 | 3 |
| 7 | 7 | 9 | 10 | 5 | 3 |
| 8 | 8 | 9 | 3 | 9 | 10 |
| 8 | 10 | 2 | 9 | 5 | 9 |
| 9 | 6 | 2 | 2 | 7 | 10 |
| 9 | 7 | 5 | 3 | 10 | 6 |
| 10 | 3 | 6 | 8 | 9 | 2 |
| 10 | 9 | 3 | 2 | 7 | 3 |
| 8 | 9 | 10 | 3 | 6 | 2 |
| 3 | 2 | 5 | 10 | 8 | 9 |
| 8 | 2 | 3 | 10 | 2 | 9 |
| 6 | 3 | 9 | 8 | 2 | 10 |
| 3 | 7 | 3 | 9 | 9 | 10 |
| 9 | 10 | 1 | 1 | 9 | 4 |
| 10 | 1 | 9 | 1 | 4 | 1 |
| 7 | 1 | 10 | 9 | 8 | 1 |
| 9 | 1 | 10 | 1 | 10 | 6 |
| 9 | 6 | 9 | 1 | 3 | 10 |
| 3 | 10 | 8 | 8 | 9 | 1 |
| 3 | 8 | 1 | 9 | 10 | 3 |
| 9 | 10 | 1 | 3 | 6 | 9 |
| 1 | 9 | 1 | 10 | 3 | 1 |
| 1 | 4 | 9 | 6 | 8 | 10 |
| 3 | 3 | 9 | 6 | 1 | 10 |
| 5 | 3 | 1 | 6 | 9 | 10 |
| 6 | 1 | 8 | 10 | 9 | 6 |
| 5 | 9 | 9 | 4 | 10 | 3 |
| 2 | 10 | 9 | 1 | 9 | 5 |
| 10 | 10 | 7 | 2 | 1 | 9 |
| 10 | 9 | 9 | 1 | 8 | 2 |
| 1 | 8 | 6 | 8 | 9 | 10 |
| 1 | 9 | 1 | 3 | 8 | 10 |
| 9 | 6 | 9 | 10 | 1 | 2 |
| 1 | 10 | 8 | 9 | 9 | 2 |
| 1 | 9 | 6 | 7 | 2 | 9 |
| 4 | 3 | 9 | 3 | 5 | 1 |
| 5 | 11 | 10 | 14 | 12 | 1 |
| 7 | 12 | 4 | 13 | 3 | 2 |
| 5 | 5 | 4 | 4 | 12 | 9 |
| 2 | 13 | 13 | 11 | 13 | 13 |
| 10 | 2 | 5 | 4 | 12 | 7 |
| 11 | 7 | 4 | 11 | 6 | 4 |
| 12 | 12 | 1 | 9 | 11 | 11 |
| 12 | 9 | 4 | 14 | 12 | 6 |
| 12 | 7 | 13 | 2 | 9 | 11 |
| 9 | 11 | 3 | 4 | 1 | 3 |
| 10 | 5 | 12 | 11 | 4 | 4 |
| 4 | 13 | 7 | 12 | 1 | 5 |
| 9 | 13 | 10 | 11 | 11 | 6 |
| 10 | 14 | 14 | 10 | 1 | 3 |
| 2 | 14 | 1 | 10 | 4 | 5 |
| 10 | 12 | 12 | 7 | 11 | 10 |
| 9 | 11 | 2 | 12 | 8 | 11 |
| 2 | 8 | 5 | 2 | 12 | 14 |
| 1 | 8 | 13 | 3 | 7 | 8 |
| 9 | 4 | 7 | 5 | 4 | 2 |
| 13 | 2 | 12 | 7 | 1 | 12 |
| 11 | 10 | 9 | 7 | 5 | 11 |
| 8 | 12 | 2 | 2 | 12 | 7 |
| 5 | 2 | 14 | 3 | 4 | 13 |
| 1 | 8 | 8 | 1 | 5 | 9 |
| 14 | 5 | 11 | 10 | 13 | 3 |
| 14 | 1 | 4 | 13 | 2 | 4 |
| 4 | 4 | 5 | 11 | 3 | 10 |
| 10 | 9 | 2 | 3 | 3 | 11 |
| 11 | 4 | 8 | 14 | 3 | 4 |
| 5 | 1 | 14 | 8 | 11 | 2 |
| 14 | 3 | 11 | 6 | 12 | 5 |
| 13 | 4 | 4 | 1 | 10 | 1 |
| 6 | 10 | 11 | 6 | 5 | 1 |
| 5 | 8 | 12 | 5 | 1 | 7 |
| 4 | 5 | 9 | 6 | 9 | 2 |
| 13 | 2 | 4 | 4 | 2 | 3 |
| 11 | 2 | 2 | 5 | 9 | 3 |
| 8 | 1 | 10 | 12 | 2 | 8 |
| 12 | 7 | 9 | 11 | 4 | 1 |
| 12 | 1 | 4 | 14 | 3 | 13 |
| 11 | 2 | 7 | 10 | 4 | 1 |
| 3 | 4 | 12 | 11 | 11 | 11 |
| 3 | 3 | 4 | 2 | 12 | 11 |
| 1 | 5 | 9 | 4 | 2 | 1 |
| 6 | 1 | 12 | 2 | 10 | 5 |
| 10 | 5 | 1 | 12 | 2 | 14 |
| 2 | 11 | 7 | 9 | 4 | 11 |
| 7 | 4 | 4 | 5 | 14 | 12 |
| 12 | 5 | 2 | 1 | 10 | 12 |
| 5 | 9 | 2 | 11 | 6 | 1 |
| 12 | 14 | 3 | 6 | 1 | 14 |
| 5 | 9 | 11 | 10 | 1 | 4 |
| 2 | 5 | 12 | 14 | 10 | 10 |
| 4 | 5 | 8 | 4 | 5 | 6 |
| 10 | 12 | 4 | 6 | 12 | 5 |
| 4 | 2 | 1 | 13 | 6 | 8 |
| 9 | 10 | 10 | 14 | 5 | 3 |
| 6 | 14 | 10 | 11 | 3 | 3 |
| 2 | 9 | 10 | 12 | 5 | 7 |
| 13 | 3 | 7 | 10 | 5 | 12 |
| 6 | 4 | 1 | 2 | 5 | 13 |
| 6 | 1 | 13 | 4 | 14 | 13 |
| 2 | 12 | 1 | 14 | 1 | 9 |
| 4 | 11 | 13 | 2 | 6 | 10 |
| 1 | 10 | 7 | 4 | 5 | 8 |
| 7 | 2 | 2 | 10 | 13 | 4 |
| 8 | 2 | 11 | 4 | 6 | 14 |
| 4 | 8 | 2 | 6 | 2 | 3 |
| 7 | 1 | 12 | 11 | 2 | 9 |
| 5 | 6 | 10 | 4 | 13 | 4 |
| 5 | 10 | 4 | 11 | 9 | 3 |
| 3 | 11 | 9 | 3 | 2 | 3 |
| 8 | 15 | 6 | 20 | 17 | 19 |
| 21 | 10 | 15 | 3 | 7 | 11 |
| 11 | 7 | 17 | 20 | 14 | 9 |
| 16 | 6 | 17 | 13 | 21 | 21 |
| 10 | 15 | 22 | 6 | 17 | 21 |
| 15 | 7 | 17 | 10 | 22 | 22 |
| 3 | 20 | 8 | 15 | 20 | 16 |
| 17 | 21 | 10 | 16 | 6 | 22 |
| 6 | 21 | 14 | 14 | 14 | 16 |
| 7 | 17 | 3 | 20 | 10 | 7 |
| 16 | 19 | 14 | 17 | 7 | 21 |
| 20 | 16 | 7 | 15 | 22 | 10 |
| 20 | 10 | 18 | 11 | 22 | 18 |
| 18 | 7 | 19 | 15 | 7 | 22 |
| 21 | 18 | 7 | 21 | 16 | 3 |
| 14 | 13 | 7 | 22 | 17 | 13 |
| 19 | 7 | 8 | 12 | 10 | 17 |
| 15 | 3 | 21 | 14 | 9 | 7 |
| 19 | 6 | 15 | 7 | 14 | 14 |
| 4 | 17 | 10 | 15 | 20 | 19 |
| 21 | 6 | 18 | 4 | 20 | 16 |
| 2 | 19 | 8 | 17 | 6 | 13 |
| 12 | 12 | 6 | 17 | 4 | 20 |
| 16 | 21 | 12 | 10 | 19 | 16 |
| 14 | 14 | 15 | 2 | 7 | 21 |
| 8 | 16 | 21 | 6 | 22 | 16 |
| 14 | 17 | 22 | 14 | 17 | 20 |
| 10 | 21 | 7 | 15 | 21 | 18 |
| 16 | 13 | 20 | 18 | 21 | 12 |
| 15 | 7 | 4 | 22 | 14 | 13 |
| 7 | 19 | 14 | 8 | 15 | 4 |
| 4 | 5 | 3 | 20 | 7 | 16 |
| 22 | 18 | 6 | 18 | 13 | 20 |
| 19 | 6 | 16 | 3 | 13 | 3 |
| 18 | 6 | 22 | 7 | 20 | 18 |
| 10 | 17 | 11 | 21 | 8 | 13 |
| 7 | 10 | 17 | 19 | 10 | 14 |

wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
(B) each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X≠Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12×L) in length;
where L=L₁, or if L₁≠L₂, where L is the greater of L₁ and L₂; and
wherein any base present may be substituted by an analogue thereof;
v) a second target nucleic acid, distinct from said first target nucleic acid, and having a fourth region, a fifth region and a sixth region, wherein said fourth region is located adjacent to and downstream from said fifth region, and said fifth region is located adjacent to and downstream from said sixth region, said fifth region having a sequence complementary to said 3′ portion of said sequence selected from the group of sequences listed in step (a)(iv), said sixth region having a sequence complementary to said 5′ portion of the sequence selected from the group of sequences in step (a)(iv);
vi) a third oligonucleotide having a 5′ portion, a central portion and a 3′ portion, said 5′ portion of said third oligonucleotide having a sequence complementary to said fourth region of said second target nucleic acid, said central portion of said third oligonucleotide having a sequence complementary to said fifth region of said second target nucleic acid, and said 3′ portion of said third oligonucleotide having a sequence that is not base paired to either said second target nucleic acid or said first target nucleic acid and is selected from a set of oligonucleotides based on the group of sequences listed in step (a)(iv) such that said sequence selected is distinct from said sequence selected in step (a)(iv);
b) mixing said cleavage means, said first target nucleic acid, said second target nucleic acid, said first, second, and third oligonucleotides to create a reaction mixture under reaction conditions such that at least said 5′ portion of said first oligonucleotide is annealed to said first target nucleic acid and wherein at least said 5′ and central portion of said second oligonucleotide is annealed to said first target nucleic acid so as to create a first cleavage structure and wherein the combined melting temperature of said complementary regions within said 5′ and 3′ portions of said first oligonucleotide when annealed to said first target nucleic acid is greater than the melting temperature of said 5′ and central portion of said first oligonucleotide, wherein cleavage of said first cleavage structure occurs to generate a first non-target cleavage product, and wherein at least said 5′ portion first non-target cleavage product is annealed to said second target nucleic acid and at least said 5′ and central portion of said third oligonucleotide is annealed to said second target nucleic acid so as to create a second cleavage structure and wherein the combined melting temperature of said complementary regions within said 5′ and 3′ portions of said non-target cleavage product when annealed to said second target nucleic acid is greater than the melting temperature of said 5′ and central portion of said third oligonucleotide, wherein cleavage of said second cleavage structure occurs to generate a second non-target cleavage product; and
c) detecting said second non-target cleavage product.

56. The method of claim 55, wherein said first target nucleic acid is genomic DNA and said second target nucleic acid is synthetic DNA.

57. The method of claim 55, wherein said synthetic DNA has at least one hairpin loop.

58. The method of claim 57, wherein the method includes a plurality of said first target nucleic acid sequences, a plurality of first oligonucleotide molecules, a plurality of said second oligonucleotide molecules, a plurality of said second target nucleic acid sequences and a plurality of third oligonucleotide molecules.

59. A method of analyzing a biological sample comprising a plurality of target nucleic acid molecules for the presence of a mutation or polymorphism at a locus of each target nucleic acid molecule, the method comprising:
a) providing:
i) a cleavage means,
ii) a first target nucleic acid, said first target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5′ portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said second oligonucleotide having a sequence that is not base-paired to said target nucleic acid and is selected from a set of oligonucleotides, based on a following group of sequences each having a 3′ and 5′ portion,

| | | | | | |
|---|---|---|---|---|---|
| 1 | 4 | 6 | 6 | 1 | 3 |
| 2 | 4 | 5 | 5 | 2 | 3 |
| 1 | 8 | 1 | 2 | 3 | 4 |
| 1 | 7 | 1 | 9 | 8 | 4 |
| 1 | 1 | 9 | 2 | 6 | 9 |
| 1 | 2 | 4 | 3 | 9 | 6 |
| 9 | 8 | 9 | 8 | 10 | 9 |
| 9 | 1 | 2 | 3 | 8 | 10 |
| 8 | 8 | 7 | 4 | 3 | 1 |
| 1 | 1 | 1 | 1 | 1 | 2 |
| 2 | 1 | 3 | 3 | 2 | 2 |
| 3 | 1 | 2 | 2 | 3 | 2 |
| 4 | 1 | 4 | 4 | 4 | 2 |
| 1 | 2 | 3 | 3 | 1 | 1 |
| 1 | 3 | 2 | 2 | 1 | 4 |
| 3 | 3 | 3 | 3 | 3 | 4 |
| 4 | 3 | 1 | 1 | 4 | 4 |
| 3 | 4 | 1 | 1 | 3 | 3 |
| 3 | 6 | 6 | 6 | 3 | 5 |
| 6 | 6 | 1 | 1 | 6 | 5 |
| 7 | 6 | 7 | 7 | 7 | 5 |
| 8 | 7 | 5 | 5 | 8 | 8 |
| 2 | 1 | 7 | 7 | 1 | 1 |
| 2 | 3 | 2 | 3 | 1 | 3 |
| 2 | 6 | 5 | 6 | 1 | 6 |
| 4 | 8 | 1 | 1 | 3 | 8 |
| 5 | 3 | 1 | 1 | 6 | 3 |
| 5 | 6 | 8 | 8 | 6 | 6 |
| 8 | 3 | 6 | 5 | 7 | 3 |
| 1 | 2 | 3 | 1 | 4 | 6 |
| 1 | 5 | 7 | 5 | 4 | 3 |
| 2 | 1 | 6 | 7 | 3 | 6 |
| 2 | 6 | 1 | 3 | 3 | 1 |
| 2 | 7 | 6 | 8 | 3 | 1 |
| 3 | 4 | 3 | 1 | 2 | 5 |
| 3 | 5 | 6 | 1 | 2 | 7 |
| 3 | 6 | 1 | 7 | 2 | 7 |
| 4 | 6 | 3 | 5 | 1 | 7 |
| 5 | 4 | 6 | 3 | 8 | 6 |
| 6 | 8 | 2 | 3 | 7 | 1 |
| 7 | 1 | 7 | 8 | 6 | 3 |
| 7 | 3 | 4 | 1 | 6 | 8 |
| 4 | 7 | 7 | 1 | 2 | 4 |
| 3 | 6 | 5 | 2 | 6 | 3 |
| 1 | 4 | 1 | 4 | 6 | 1 |
| 3 | 3 | 1 | 4 | 8 | 1 |
| 8 | 3 | 3 | 5 | 3 | 8 |
| 1 | 3 | 6 | 6 | 3 | 7 |
| 7 | 3 | 8 | 6 | 4 | 7 |
| 3 | 1 | 3 | 7 | 8 | 6 |
| 10 | 9 | 5 | 5 | 10 | 10 |
| 7 | 10 | 10 | 10 | 7 | 9 |
| 9 | 9 | 7 | 7 | 10 | 9 |
| 9 | 3 | 10 | 3 | 10 | 3 |
| 9 | 6 | 3 | 4 | 10 | 6 |
| 10 | 4 | 10 | 3 | 9 | 4 |
| 3 | 9 | 3 | 10 | 4 | 9 |
| 9 | 10 | 5 | 9 | 4 | 8 |
| 3 | 9 | 4 | 9 | 10 | 7 |
| 3 | 5 | 9 | 4 | 10 | 8 |
| 4 | 10 | 5 | 4 | 9 | 3 |
| 5 | 3 | 3 | 9 | 8 | 10 |
| 6 | 8 | 6 | 9 | 7 | 10 |
| 4 | 6 | 10 | 9 | 6 | 4 |
| 4 | 9 | 8 | 10 | 8 | 3 |
| 7 | 7 | 9 | 10 | 5 | 3 |
| 8 | 8 | 9 | 3 | 9 | 10 |
| 8 | 10 | 2 | 9 | 5 | 9 |
| 9 | 6 | 2 | 2 | 7 | 10 |
| 9 | 7 | 5 | 3 | 10 | 6 |
| 10 | 3 | 6 | 8 | 9 | 2 |
| 10 | 9 | 3 | 2 | 7 | 3 |
| 8 | 9 | 10 | 3 | 6 | 2 |
| 3 | 2 | 5 | 10 | 8 | 9 |
| 8 | 2 | 3 | 10 | 2 | 9 |
| 6 | 3 | 9 | 8 | 2 | 10 |
| 3 | 7 | 3 | 9 | 9 | 10 |
| 9 | 10 | 1 | 1 | 9 | 4 |
| 10 | 1 | 9 | 1 | 4 | 1 |
| 7 | 1 | 10 | 9 | 8 | 1 |
| 9 | 1 | 10 | 1 | 10 | 6 |
| 9 | 6 | 9 | 1 | 3 | 10 |
| 3 | 10 | 8 | 8 | 9 | 1 |
| 3 | 8 | 1 | 9 | 10 | 3 |
| 9 | 10 | 1 | 3 | 6 | 9 |
| 1 | 9 | 1 | 10 | 3 | 1 |
| 1 | 4 | 9 | 6 | 8 | 10 |
| 3 | 3 | 9 | 6 | 1 | 10 |
| 5 | 3 | 1 | 6 | 9 | 10 |
| 6 | 1 | 8 | 10 | 9 | 6 |
| 5 | 9 | 9 | 4 | 10 | 3 |
| 2 | 10 | 9 | 1 | 9 | 5 |
| 10 | 10 | 7 | 2 | 1 | 9 |
| 10 | 9 | 9 | 1 | 8 | 2 |
| 1 | 8 | 6 | 8 | 9 | 10 |
| 1 | 9 | 1 | 3 | 8 | 10 |
| 9 | 6 | 9 | 10 | 1 | 2 |
| 1 | 10 | 8 | 9 | 9 | 2 |
| 1 | 9 | 6 | 7 | 2 | 9 |
| 4 | 3 | 9 | 3 | 5 | 1 |
| 5 | 11 | 10 | 14 | 12 | 1 |
| 7 | 12 | 4 | 13 | 3 | 2 |
| 5 | 5 | 4 | 4 | 12 | 9 |
| 2 | 13 | 13 | 11 | 13 | 13 |
| 10 | 2 | 5 | 4 | 12 | 7 |
| 11 | 7 | 4 | 11 | 6 | 4 |
| 12 | 12 | 1 | 9 | 11 | 11 |
| 12 | 9 | 4 | 14 | 12 | 6 |
| 12 | 7 | 13 | 2 | 9 | 11 |
| 9 | 11 | 3 | 4 | 1 | 3 |
| 10 | 5 | 12 | 11 | 4 | 4 |
| 4 | 13 | 7 | 12 | 1 | 5 |
| 9 | 13 | 10 | 11 | 11 | 6 |
| 10 | 14 | 14 | 10 | 1 | 3 |
| 2 | 14 | 1 | 10 | 4 | 5 |
| 10 | 12 | 12 | 7 | 11 | 10 |
| 9 | 11 | 2 | 12 | 8 | 11 |
| 2 | 8 | 5 | 2 | 12 | 14 |
| 1 | 8 | 13 | 3 | 7 | 8 |
| 9 | 4 | 7 | 5 | 4 | 2 |
| 13 | 2 | 12 | 7 | 1 | 12 |
| 11 | 10 | 9 | 7 | 5 | 11 |
| 8 | 12 | 2 | 2 | 12 | 7 |
| 5 | 2 | 14 | 3 | 4 | 13 |
| 1 | 8 | 8 | 1 | 5 | 9 |
| 14 | 5 | 11 | 10 | 13 | 3 |
| 14 | 1 | 4 | 13 | 2 | 4 |
| 4 | 4 | 5 | 11 | 3 | 10 |
| 10 | 9 | 2 | 3 | 3 | 11 |
| 11 | 4 | 8 | 14 | 3 | 4 |
| 5 | 1 | 14 | 8 | 11 | 2 |
| 14 | 3 | 11 | 6 | 12 | 5 |
| 13 | 4 | 4 | 1 | 10 | 1 |
| 6 | 10 | 11 | 6 | 5 | 1 |
| 5 | 8 | 12 | 5 | 1 | 7 |
| 4 | 5 | 9 | 6 | 9 | 2 |
| 13 | 2 | 4 | 4 | 2 | 3 |
| 11 | 2 | 2 | 5 | 9 | 3 |
| 8 | 1 | 10 | 12 | 2 | 8 |
| 12 | 7 | 9 | 11 | 4 | 1 |
| 12 | 1 | 4 | 14 | 3 | 13 |
| 11 | 2 | 7 | 10 | 4 | 1 |
| 3 | 4 | 12 | 11 | 11 | 11 |
| 3 | 3 | 4 | 2 | 12 | 11 |
| 1 | 5 | 9 | 4 | 2 | 1 |
| 6 | 1 | 12 | 2 | 10 | 5 |
| 10 | 5 | 1 | 12 | 2 | 14 |
| 2 | 11 | 7 | 9 | 4 | 11 |
| 7 | 4 | 4 | 5 | 14 | 12 |
| 12 | 5 | 2 | 1 | 10 | 12 |
| 5 | 9 | 2 | 11 | 6 | 1 |
| 12 | 14 | 3 | 6 | 1 | 14 |
| 5 | 9 | 11 | 10 | 1 | 4 |
| 2 | 5 | 12 | 14 | 10 | 10 |
| 4 | 5 | 8 | 4 | 5 | 6 |
| 10 | 12 | 4 | 6 | 12 | 5 |
| 4 | 2 | 1 | 13 | 6 | 8 |
| 9 | 10 | 10 | 14 | 5 | 3 |
| 6 | 14 | 10 | 11 | 3 | 3 |
| 2 | 9 | 10 | 12 | 5 | 7 |
| 13 | 3 | 7 | 10 | 5 | 12 |
| 6 | 4 | 1 | 2 | 5 | 13 |
| 6 | 1 | 13 | 4 | 14 | 13 |
| 2 | 12 | 1 | 14 | 1 | 9 |
| 4 | 11 | 13 | 2 | 6 | 10 |
| 1 | 10 | 7 | 4 | 5 | 8 |
| 7 | 2 | 2 | 10 | 13 | 4 |
| 8 | 2 | 11 | 4 | 6 | 14 |
| 4 | 8 | 2 | 6 | 2 | 3 |
| 7 | 1 | 12 | 11 | 2 | 9 |
| 5 | 6 | 10 | 4 | 13 | 4 |
| 5 | 10 | 4 | 11 | 9 | 3 |
| 3 | 11 | 9 | 3 | 2 | 3 |
| 8 | 15 | 6 | 20 | 17 | 19 |
| 21 | 10 | 15 | 3 | 7 | 11 |
| 11 | 7 | 17 | 20 | 14 | 9 |
| 16 | 6 | 17 | 13 | 21 | 21 |
| 10 | 15 | 22 | 6 | 17 | 21 |
| 15 | 7 | 17 | 10 | 22 | 22 |
| 3 | 20 | 8 | 15 | 20 | 16 |
| 17 | 21 | 10 | 16 | 6 | 22 |
| 6 | 21 | 14 | 14 | 14 | 16 |
| 7 | 17 | 3 | 20 | 10 | 7 |
| 16 | 19 | 14 | 17 | 7 | 21 |
| 20 | 16 | 7 | 15 | 22 | 10 |
| 20 | 10 | 18 | 11 | 22 | 18 |
| 18 | 7 | 19 | 15 | 7 | 22 |
| 21 | 18 | 7 | 21 | 16 | 3 |
| 14 | 13 | 7 | 22 | 17 | 13 |
| 19 | 7 | 8 | 12 | 10 | 17 |
| 15 | 3 | 21 | 14 | 9 | 7 |
| 19 | 6 | 15 | 7 | 14 | 14 |
| 4 | 17 | 10 | 15 | 20 | 19 |
| 21 | 6 | 18 | 4 | 20 | 16 |
| 2 | 19 | 8 | 17 | 6 | 13 |
| 12 | 12 | 6 | 17 | 4 | 20 |
| 16 | 21 | 12 | 10 | 19 | 16 |
| 14 | 14 | 15 | 2 | 7 | 21 |
| 8 | 16 | 21 | 6 | 22 | 16 |
| 14 | 17 | 22 | 14 | 17 | 20 |
| 10 | 21 | 7 | 15 | 21 | 18 |
| 16 | 13 | 20 | 18 | 21 | 12 |
| 15 | 7 | 4 | 22 | 14 | 13 |
| 7 | 19 | 14 | 8 | 15 | 4 |
| 4 | 5 | 3 | 20 | 7 | 16 |
| 22 | 18 | 6 | 18 | 13 | 20 |
| 19 | 6 | 16 | 3 | 13 | 3 |
| 18 | 6 | 22 | 7 | 20 | 18 |
| 10 | 17 | 11 | 21 | 8 | 13 |
| 7 | 10 | 17 | 19 | 10 | 14 |

wherein:
(A) each of 1 to 22 is a 4mer selected from the group of 4mers consisting of WWWW, WWWX, WWWY, WWXW, WWXX, WWXY, WWYW, WWYX, WWYY, WXWW, WXWX, WXWY, WXXW, WXXX, WXXY, WXYW, WXYX, WXYY, WYWW, WYWX, WYWY, WYXW, WYXX, WYXY, WYYW, WYYX, WYYY, XWWW, XWWX, XWWY, XWXW, XWXX, XWXY, XWYW, XWYX, XWYY, XXWW, XXWX, XXWY, XXXW, XXXX, XXXY, XXYW, XXYX, XXYY, XYWW, XYWX, XYWY, XYXW, XYXX, XYXY, XYYW, XYYX, XYYY, YWWW, YWWX, YWWY, YWXW, YWXX, YWXY, YWYW, YWYX, YWYY, YXWW, YXWX, YXWY, YXXW, YXXX, YXXY, YXYW, YXYX, YXYY, YYWW, YYWX, YYWY, YYXW, YYXX, YYXY, YYYW, YYYX, and YYYY, and
(B) each of 1 to 22 is selected so as to be different from all of the others of 1 to 22;
each of W, X and Y is a base in which:
(i) (a) W=one of A, T/U, G, and C,
X=one of A, T/U, G, and C,
Y=one of A, T/U, G, and C,
and each of W, X and Y is selected so as to be different from all of the others of W, X and Y,
(b) an unselected said base of (i)(a) can be substituted any number of times for any one of W, X and Y, or
(ii) (a) W=G or C,
X=A or T/U,
Y=A or T/U,
and X═Y, and
(b) a base not selected in (ii)(a) can be inserted into each sequence at one or more locations, the location of each insertion being the same in all the sequences;
(D) up to three bases can be inserted at any location of any of the sequences or up to three bases can be deleted from any of the sequences;
(E) all of the sequences of a said group of oligonucleotides are read 5′ to 3′ or are read 3′ to 5′; and
wherein each oligonucleotide of a said set has a sequence of at least ten contiguous bases of the sequence on which it is based, provided that:
(F) (I) the quotient of the sum of G and C divided by the sum of A, T/U, G and C for all combined sequences of the set is between about 0.1 and 0.40 and said quotient for each sequence of the set does not vary from the quotient for the combined sequences by more than 0.2; and
(II) for any phantom sequence generated from any pair of first and second sequences of the set L₁ and L₂ in length, respectively, by selection from the first and second sequences of identical bases in identical sequence with each other:
(i) any consecutive sequence of bases in the phantom sequence which is identical to a consecutive sequence of bases in each of the first and second sequences from which it is generated is less than ((¾×L)−1) bases in length;
(ii) the phantom sequence, if greater than or equal to (⅚×L) in length, contains at least three insertions/deletions or mismatches when compared to the first and second sequences from which it is generated; and
(iii) the phantom sequence is not greater than or equal to ( 11/12×L) in length;
where L=L₁, or if L₁≠L₂, where L is the greater of L₁ and L₂; and
wherein any base present may be substituted by an analogue thereof;
v) a second target nucleic acid, distinct from said first target nucleic acid, and having a fourth region, a fifth region and a sixth region, wherein said fourth region is located adjacent to and downstream from said fifth region, and said fifth region is located adjacent to and downstream from said sixth region, said fifth region having a sequence complementary to said 3′ portion of said sequence selected from the group of sequences listed in step (a)(iv), said sixth region having a sequence complementary to said 5′ portion of the sequence selected from the group of sequences in step (a)(iv);
vi) a third oligonucleotide having a 5′ portion, a central portion and a 3′ portion, said 5′ portion of said third oligonucleotide having a sequence complementary to said fourth region of said second target nucleic acid, said central portion of said third oligonucleotide having a sequence complementary to said fifth region of said second target nucleic acid, and said 3′ portion of said third oligonucleotide having a sequence that is not base paired to either said second target nucleic acid or said first target nucleic acid and is selected from a set of oligonucleotides based on the group of sequences listed in step (a)(iv) such that said sequence selected is distinct from said sequence selected in step (a)(iv); and
b) mixing said cleavage means, said first target nucleic acid, said second target nucleic acid, said first, second, and third oligonucleotides to create a reaction mixture under reaction conditions such that at least said 5′ portion of said first oligonucleotide is annealed to said first target nucleic acid and wherein at least said 5′ and central portion of said second oligonucleotide is annealed to said first target nucleic acid so as to create a first cleavage structure and wherein the combined melting temperature of said complementary regions within said 5′ and 3′ portions of said first oligonucleotide when annealed to said first target nucleic acid is greater than the melting temperature of said 5′ and central portion of said first oligonucleotide, wherein cleavage of said first cleavage structure occurs to generate a first non-target cleavage product, and wherein at least said 5′ portion first non-target cleavage product is annealed to said second target nucleic acid and at least said 5′ and central portion of said third oligonucleotide is annealed to said second target nucleic acid so as to create a second cleavage structure and wherein the combined melting temperature of said complementary regions within said 5′ and 3′ portions of said non-target cleavage product when annealed to said second target nucleic acid is greater than the melting temperature of said 5′ and central portion of said third oligonucleotide, wherein cleavage of said second cleavage structure occurs to generate a second non-target cleavage product; and
c) detecting said second non-target cleavage product.

60. The method of claim 59, wherein said first target nucleic acid is genomic DNA and said second target nucleic acid is synthetic DNA.

61. The method of claim 60, wherein said synthetic DNA has at least one hairpin loop.

62. The method of claim 61, wherein the method includes a plurality of said first target nucleic acid sequences, a plurality of first oligonucleotide molecules, a plurality of said second oligonucleotide molecules, a plurality of said second target nucleic acid sequences and a plurality of third oligonucleotide molecules.

63. A method of detecting the presence of a target nucleic acid molecule by detecting non-target cleavage products, the method comprising:
a) providing:
i) a cleavage means,
ii) a first target nucleic acid, said first target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5′ portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said second oligonucleotide having a sequence that is not base-paired to said target nucleic acid and is selected from a set of oligonucleotides, based on a following group of sequences each having a 3′ and 5′ portion,

| | | | | | | | | | | | | | | | | | | | | | | | |
|---|---|---|---|---|---|---|---|---|---|---|---|---|---|---|---|---|---|---|---|---|---|---|---|
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | 1 | 1 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

Wherein each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3≦20, M4≦16, and M5≦19, where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein:
the score of an alignment is determined according to the equation (A×m)−(B×mm)−(C×(og+eg))−(D×eg)), wherein:
for each of (i) to (iv):
(i) m=6, mm=6, og=0 and eg=6,
(ii) m=6, mm=6, og=5 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv).
v) a second target nucleic acid, distinct from said first target nucleic acid, and having a fourth region, a fifth region and a sixth region, wherein said fourth region is located adjacent to and downstream from said fifth region, and said fifth region is located adjacent to and downstream from said sixth region, said fifth region having a sequence complementary to said 3′ portion of said sequence selected from the group of sequences listed in step (a)(iv), said sixth region having a sequence complementary to said 5′ portion of the sequence selected from the group of sequences in step (a)(iv);
vi) a third oligonucleotide having a 5′ portion, a central portion and a 3′ portion, said 5′ portion of said third oligonucleotide having a sequence complementary to said fourth region of said second target nucleic acid, said central portion of said third oligonucleotide having a sequence complementary to said fifth region of said second target nucleic acid, and said 3′ portion of said third oligonucleotide having a sequence that is not base paired to either said second target nucleic acid or said first target nucleic acid and is selected from a set of oligonucleotides based on the group of sequences listed in step (a)(iv) such that said sequence selected is distinct from said sequence selected in step (a)(iv);
b) mixing said cleavage means, said first target nucleic acid, said second target nucleic acid, said first, second, and third oligonucleotides to create a reaction mixture under reaction conditions such that at least said 5′ portion of said first oligonucleotide is annealed to said first target nucleic acid and wherein at least said 5′ and central portion of said second oligonucleotide is annealed to said first target nucleic acid so as to create a first cleavage structure and wherein the combined melting temperature of said complementary regions within said 5′ and 3′ portions of said first oligonucleotide when annealed to said first target nucleic acid is greater than the melting temperature of said 5′ and central portion of said first oligonucleotide, wherein cleavage of said first cleavage structure occurs to generate a first non-target cleavage product, and wherein at least said 5′ portion first non-target cleavage product is annealed to said second target nucleic acid and at least said 5′ and central portion of said third oligonucleotide is annealed to said second target nucleic acid so as to create a second cleavage structure and wherein the combined melting temperature of said complementary regions within said 5′ and 3′ portions of said non-target cleavage product when annealed to said second target nucleic acid is greater than the melting temperature of said 5′ and central portion of said third oligonucleotide, wherein cleavage of said second cleavage structure occurs to generate a second non-target cleavage product; and
c) detecting said second non-target cleavage product.

64. The method of claim 63, wherein said first target nucleic acid is genomic DNA and said second target nucleic acid is synthetic DNA.

65. The method of claim 64, wherein said synthetic DNA has at least one hairpin loop.

66. The method of claim 65, wherein the method includes a plurality of said first target nucleic acid sequences, a plurality of first oligonucleotide molecules, a plurality of said second oligonucleotide molecules, a plurality of said second target nucleic acid sequences and a plurality of third oligonucleotide molecules.

67. A method of analyzing a biological sample comprising a plurality of target nucleic acid molecules for the presence of a mutation or polymorphism at a locus of each target nucleic acid molecule, the method comprising:
a) providing:
i) a cleavage means,
ii) a first target nucleic acid, said first target nucleic acid having a first region, a second region and a third region, wherein said first region is located adjacent to and downstream from said second region, and said second region is located adjacent to and downstream from said third region;
iii) a first oligonucleotide having a 5′ and a 3′ portion, said 5′ portion of said first oligonucleotide having a sequence complementary to said second region of said target nucleic acid and said 3′ portion of said first oligonucleotide having a sequence complementary to said third region of said target nucleic acid;
iv) a second oligonucleotide having a 5′ portion, a central portion and a 3′ portion, said 5′ portion of said second oligonucleotide having a sequence complementary to said first region of said target nucleic acid, said central portion of said second oligonucleotide having a sequence complimentary to said second region of said target nucleic acid, and said 3′ portion of said 20 second oligonucleotide having a sequence that is not base-paired to said target nucleic acid and is selected from a set of oligonucleotides, based on a following group of sequences each having a 3′ and 5′ portion,

| | | | | | | | | | | | | | | | | | | | | | | | |
|---|---|---|---|---|---|---|---|---|---|---|---|---|---|---|---|---|---|---|---|---|---|---|---|
| 1 | 1 | 1 | 2 | 2 | 3 | 2 | 3 | 1 | 1 | 1 | 3 | 1 | 2 | 2 | 3 | 2 | 2 | 2 | 3 | 2 | 3 | 2 | 1 |
| 3 | 2 | 2 | 1 | 3 | 1 | 3 | 2 | 2 | 1 | 1 | 2 | 2 | 3 | 2 | 1 | 2 | 2 | 2 | 3 | 1 | 2 | 3 | 1 |
| 1 | 2 | 3 | 2 | 2 | 1 | 1 | 1 | 3 | 2 | 1 | 1 | 3 | 2 | 3 | 2 | 2 | 3 | 1 | 1 | 1 | 2 | 3 | 2 |
| 2 | 3 | 1 | 2 | 3 | 2 | 2 | 1 | 3 | 1 | 1 | 3 | 2 | 1 | 2 | 1 | 2 | 2 | 3 | 2 | 3 | 1 | 1 | 2 |
| 2 | 2 | 2 | 3 | 2 | 3 | 2 | 1 | 3 | 1 | 1 | 2 | 1 | 2 | 3 | 2 | 3 | 2 | 2 | 3 | 2 | 2 | 1 | 1 |
| 1 | 2 | 1 | 1 | 3 | 2 | 3 | 2 | 1 | 1 | 3 | 2 | 3 | 1 | 1 | 1 | 2 | 1 | 1 | 3 | 1 | 1 | 3 | 1 |
| 1 | 1 | 3 | 1 | 3 | 2 | 1 | 2 | 2 | 2 | 3 | 2 | 2 | 3 | 2 | 3 | 1 | 3 | 2 | 2 | 1 | 1 | 1 | 2 |
| 3 | 2 | 3 | 2 | 2 | 2 | 1 | 2 | 3 | 2 | 2 | 1 | 2 | 1 | 2 | 3 | 2 | 3 | 1 | 1 | 3 | 2 | 2 | 2 |
| 1 | 1 | 1 | 3 | 1 | 3 | 1 | 1 | 2 | 1 | 3 | 1 | 1 | 2 | 1 | 2 | 3 | 2 | 3 | 2 | 1 | 1 | 3 | 2 |
| 2 | 1 | 2 | 3 | 1 | 1 | 1 | 3 | 1 | 3 | 2 | 3 | 1 | 3 | 1 | 2 | 1 | 1 | 2 | 3 | 2 | 2 | 2 | 1 |
| 1 | 2 | 3 | 1 | 3 | 1 | 1 | 1 | 2 | 1 | 2 | 3 | 2 | 2 | 1 | 3 | 1 | 1 | 2 | 3 | 2 | 3 | 1 | 2 |
| 2 | 2 | 1 | 3 | 2 | 2 | 3 | 2 | 2 | 3 | 1 | 2 | 3 | 2 | 2 | 2 | 1 | 3 | 2 | 1 | 3 | 2 | 2 | 2 |
| 3 | 2 | 1 | 1 | 1 | 3 | 1 | 3 | 2 | 1 | 2 | 1 | 1 | 3 | 2 | 2 | 2 | 3 | 1 | 2 | 3 | 1 | 2 | 1 |
| 1 | 1 | 1 | 3 | 2 | 1 | 1 | 3 | 1 | 1 | 2 | 3 | 1 | 2 | 3 | 2 | 1 | 1 | 2 | 1 | 1 | 3 | 2 | 3 |
| 3 | 2 | 1 | 3 | 1 | 1 | 1 | 2 | 1 | 3 | 2 | 2 | 2 | 1 | 2 | 2 | 3 | 1 | 2 | 3 | 1 | 2 | 2 | 3 |
| 2 | 3 | 2 | 1 | 1 | 3 | 2 | 3 | 1 | 1 | 1 | 2 | 1 | 3 | 2 | 3 | 1 | 3 | 2 | 2 | 1 | 2 | 2 | 2 |
| 1 | 1 | 1 | 2 | 1 | 3 | 1 | 2 | 3 | 1 | 2 | 1 | 2 | 1 | 1 | 3 | 2 | 3 | 1 | 3 | 1 | 1 | 2 | 3 |
| 1 | 2 | 1 | 1 | 3 | 2 | 2 | 1 | 2 | 1 | 1 | 3 | 2 | 3 | 2 | 2 | 1 | 2 | 3 | 2 | 3 | 1 | 3 | 2 |
| 2 | 1 | 2 | 1 | 3 | 1 | 2 | 1 | 1 | 1 | 3 | 1 | 3 | 1 | 2 | 3 | 1 | 2 | 2 | 2 | 3 | 2 | 2 | 3 |
| 1 | 3 | 1 | 3 | 2 | 2 | 3 | 1 | 3 | 1 | 1 | 2 | 3 | 2 | 1 | 2 | 1 | 3 | 2 | 1 | 2 | 2 | 1 | 2 |
| 1 | 1 | 3 | 2 | 1 | 3 | 2 | 2 | 2 | 3 | 2 | 1 | 1 | 3 | 1 | 1 | 2 | 3 | 1 | 2 | 2 | 3 | 2 | 1 |
| 2 | 2 | 1 | 2 | 3 | 1 | 1 | 1 | 2 | 2 | 3 | 1 | 3 | 2 | 3 | 1 | 1 | 3 | 1 | 2 | 2 | 3 | 1 | 2 |
| 3 | 2 | 1 | 2 | 1 | 2 | 3 | 2 | 1 | 1 | 1 | 2 | 2 | 3 | 2 | 2 | 1 | 2 | 3 | 2 | 2 | 3 | 1 | 3 |
| 3 | 1 | 1 | 2 | 2 | 3 | 2 | 1 | 2 | 1 | 1 | 1 | 3 | 2 | 1 | 2 | 2 | 1 | 3 | 1 | 2 | 3 | 2 | 3 |
| 2 | 1 | 3 | 1 | 2 | 3 | 1 | 3 | 1 | 2 | 2 | 1 | 1 | 3 | 2 | 3 | 2 | 2 | 1 | 2 | 2 | 2 | 3 | 1 |
| 3 | 2 | 2 | 1 | 1 | 3 | 2 | 2 | 2 | 3 | 2 | 2 | 2 | 1 | 2 | 3 | 2 | 1 | 2 | 1 | 3 | 1 | 1 | 3 |
| 3 | 1 | 3 | 2 | 1 | 2 | 2 | 1 | 3 | 2 | 1 | 1 | 1 | 3 | 2 | 3 | 1 | 2 | 1 | 2 | 3 | 1 | 2 | 1 |
| 3 | 2 | 3 | 1 | 1 | 2 | 3 | 1 | 2 | 2 | 2 | 1 | 3 | 2 | 1 | 1 | 1 | 2 | 3 | 1 | 2 | 2 | 3 | 1 |
| 3 | 1 | 2 | 2 | 3 | 1 | 1 | 3 | 2 | 2 | 1 | 2 | 1 | 3 | 1 | 1 | 1 | 2 | 3 | 1 | 2 | 2 | 1 | 3 |
| 1 | 3 | 2 | 3 | 1 | 2 | 1 | 1 | 1 | 2 | 3 | 2 | 2 | 1 | 3 | 2 | 2 | 3 | 1 | 1 | 2 | 2 | 3 | 2 |
| 2 | 1 | 2 | 1 | 2 | 1 | 3 | 2 | 1 | 1 | 1 | 2 | 3 | 2 | 2 | 2 | 3 | 2 | 3 | 2 | 3 | 2 | 2 | 3 |
| 2 | 2 | 1 | 1 | 3 | 2 | 3 | 2 | 2 | 1 | 3 | 2 | 2 | 1 | 2 | 2 | 2 | 3 | 2 | 2 | 3 | 2 | 1 | 3 |
| 3 | 2 | 1 | 3 | 2 | 1 | 1 | 2 | 1 | 2 | 3 | 1 | 1 | 3 | 2 | 3 | 1 | 3 | 1 | 1 | 2 | 1 | 2 | 1 |
| 2 | 1 | 3 | 2 | 3 | 2 | 1 | 2 | 1 | 3 | 1 | 1 | 2 | 3 | 2 | 1 | 3 | 1 | 2 | 2 | 2 | 1 | 3 | 2 |
| 2 | 2 | 3 | 2 | 1 | 3 | 1 | 2 | 2 | 1 | 3 | 1 | 2 | 3 | 2 | 3 | 2 | 2 | 2 | 3 | 2 | 1 | 1 | 1 |
| 2 | 1 | 3 | 2 | 1 | 2 | 1 | 3 | 1 | 3 | 2 | 1 | 3 | 1 | 3 | 1 | 2 | 3 | 1 | 2 | 1 | 2 | 2 | 2 |
| 1 | 2 | 2 | 3 | 2 | 3 | 1 | 1 | 1 | 3 | 1 | 1 | 1 | 3 | 1 | 3 | 1 | 1 | 3 | 1 | 1 | 1 | 2 | 2 |
| 2 | 3 | 2 | 3 | 1 | 3 | 1 | 1 | 2 | 2 | 1 | 1 | 3 | 1 | 2 | 2 | 1 | 1 | 3 | 1 | 1 | 2 | 3 | 2 |
| 1 | 2 | 1 | 2 | 2 | 1 | 3 | 2 | 2 | 1 | 1 | 3 | 1 | 1 | 1 | 3 | 1 | 1 | 3 | 1 | 3 | 2 | 2 | 3 |
| 2 | 2 | 3 | 2 | 1 | 3 | 2 | 2 | 3 | 1 | 3 | 1 | 1 | 1 | 2 | 1 | 2 | 3 | 2 | 1 | 3 | 2 | 2 | 2 |
| 2 | 1 | 3 | 1 | 3 | 2 | 2 | 3 | 2 | 2 | 1 | 1 | 1 | 3 | 1 | 3 | 2 | 3 | 2 | 1 | 1 | 1 | 2 | 1 |
| 3 | 2 | 2 | 1 | 2 | 3 | 1 | 2 | 3 | 2 | 3 | 2 | 1 | 2 | 1 | 1 | 3 | 2 | 1 | 1 | 2 | 1 | 2 | 3 |
| 2 | 2 | 2 | 3 | 2 | 2 | 1 | 3 | 1 | 1 | 2 | 3 | 1 | 3 | 1 | 1 | 3 | 1 | 2 | 2 | 2 | 1 | 2 | 3 |
| 1 | 3 | 2 | 1 | 2 | 1 | 3 | 2 | 2 | 2 | 1 | 1 | 1 | 3 | 1 | 1 | 3 | 2 | 1 | 3 | 2 | 1 | 3 | 1 |
| 3 | 2 | 3 | 1 | 3 | 1 | 2 | 1 | 2 | 1 | 3 | 1 | 2 | 2 | 2 | 1 | 3 | 1 | 1 | 1 | 3 | 2 | 1 | 1 |
| 2 | 2 | 3 | 2 | 2 | 2 | 1 | 2 | 1 | 3 | 2 | 3 | 1 | 1 | 3 | 2 | 3 | 1 | 1 | 2 | 1 | 3 | 2 | 1 |
| 1 | 1 | 3 | 2 | 1 | 1 | 3 | 2 | 1 | 3 | 2 | 1 | 1 | 2 | 1 | 3 | 2 | 3 | 2 | 3 | 2 | 2 | 1 | 1 |
| 1 | 2 | 2 | 2 | 3 | 2 | 3 | 1 | 3 | 2 | 2 | 1 | 2 | 3 | 1 | 1 | 1 | 3 | 1 | 2 | 1 | 1 | 3 | 1 |
| 3 | 1 | 1 | 1 | 3 | 2 | 1 | 3 | 1 | 3 | 1 | 1 | 2 | 1 | 1 | 1 | 3 | 1 | 2 | 1 | 1 | 3 | 1 | 1 |
| 1 | 2 | 2 | 2 | 1 | 1 | 3 | 1 | 2 | 2 | 3 | 2 | 2 | 1 | 1 | 3 | 1 | 3 | 2 | 1 | 3 | 1 | 1 | 3 |
| 3 | 2 | 2 | 2 | 1 | 1 | 1 | 3 | 1 | 2 | 2 | 3 | 2 | 1 | 1 | 3 | 1 | 1 | 2 | 3 | 2 | 3 | 2 | 1 |
| 2 | 2 | 2 | 3 | 2 | 3 | 1 | 1 | 3 | 1 | 2 | 3 | 1 | 1 | 3 | 2 | 1 | 2 | 2 | 2 | 3 | 2 | 1 | 2 |
| 2 | 3 | 2 | 3 | 2 | 2 | 2 | 1 | 3 | 1 | 1 | 2 | 2 | 2 | 1 | 3 | 2 | 1 | 2 | 3 | 2 | 3 | 2 | 1 |
| 3 | 1 | 2 | 1 | 1 | 2 | 3 | 1 | 2 | 2 | 1 | 2 | 1 | 3 | 1 | 1 | 1 | 3 | 2 | 3 | 2 | 2 | 2 | 3 |
| 3 | 2 | 2 | 1 | 2 | 2 | 2 | 3 | 2 | 1 | 1 | 3 | 2 | 2 | 1 | 1 | 3 | 1 | 2 | 1 | 3 | 2 | 1 | 3 |
| 1 | 3 | 2 | 2 | 2 | 1 | 2 | 2 | 3 | 1 | 1 | 1 | 3 | 1 | 3 | 2 | 2 | 2 | 3 | 1 | 1 | 2 | 1 | 3 |
| 2 | 2 | 3 | 2 | 3 | 2 | 2 | 2 | 1 | 2 | 2 | 3 | 2 | 3 | 2 | 1 | 3 | 2 | 2 | 2 | 1 | 1 | 1 | 3 |
| 1 | 2 | 2 | 3 | 2 | 3 | 1 | 3 | 1 | 1 | 3 | 1 | 2 | 1 | 2 | 3 | 1 | 1 | 1 | 3 | 2 | 2 | 1 | 2 |
| 2 | 3 | 1 | 3 | 1 | 1 | 2 | 3 | 2 | 1 | 1 | 1 | 3 | 1 | 1 | 2 | 3 | 2 | 2 | 2 | 1 | 2 | 2 | 3 |
| 1 | 2 | 3 | 2 | 3 | 1 | 1 | 1 | 3 | 2 | 2 | 1 | 2 | 3 | 1 | 2 | 3 | 2 | 2 | 1 | 1 | 2 | 2 | 3 |
| 3 | 2 | 2 | 2 | 1 | 3 | 2 | 1 | 2 | 2 | 1 | 3 | 2 | 2 | 3 | 2 | 2 | 1 | 1 | 3 | 1 | 2 | 2 | 3 |
| 3 | 1 | 2 | 2 | 3 | 1 | 2 | 1 | 2 | 2 | 2 | 3 | 1 | 1 | 2 | 3 | 2 | 2 | 2 | 3 | 2 | 2 | 2 | 3 |
| 2 | 3 | 1 | 1 | 2 | 2 | 3 | 1 | 1 | 1 | 3 | 2 | 3 | 2 | 1 | 1 | 2 | 3 | 2 | 2 | 3 | 2 | 1 | 2 |
| 3 | 1 | 2 | 2 | 3 | 2 | 1 | 2 | 2 | 3 | 2 | 2 | 3 | 1 | 3 | 1 | 1 | 2 | 1 | 3 | 1 | 1 | 2 | 1 |
| 1 | 1 | 1 | 2 | 2 | 2 | 3 | 1 | 3 | 1 | 2 | 2 | 2 | 3 | 2 | 3 | 1 | 2 | 1 | 3 | 1 | 3 | 2 | 1 |
| 3 | 2 | 1 | 1 | 2 | 2 | 1 | 3 | 1 | 2 | 2 | 2 | 3 | 2 | 2 | 2 | 3 | 2 | 2 | 3 | 2 | 2 | 3 | 2 |
| 3 | 2 | 2 | 2 | 3 | 2 | 1 | 2 | 2 | 3 | 2 | 2 | 1 | 3 | 2 | 3 | 1 | 1 | 2 | 1 | 2 | 1 | 3 | 2 |
| 1 | 2 | 3 | 2 | 1 | 3 | 2 | 1 | 3 | 2 | 1 | 3 | 1 | 2 | 3 | 2 | 2 | 2 | 1 | 2 | 3 | 1 | 1 | 2 |
| 2 | 3 | 2 | 2 | 2 | 1 | 1 | 1 | 3 | 1 | 2 | 3 | 1 | 2 | 2 | 3 | 1 | 1 | 3 | 1 | 1 | 1 | 2 | 3 |
| 2 | 3 | 2 | 3 | 1 | 2 | 1 | 1 | 2 | 3 | 1 | 2 | 3 | 2 | 2 | 1 | 2 | 2 | 2 | 3 | 2 | 3 | 2 | 1 |
| 1 | 2 | 1 | 3 | 2 | 2 | 3 | 2 | 3 | 1 | 3 | 1 | 1 | 2 | 2 | 2 | 3 | 2 | 1 | 1 | 2 | 2 | 1 | 3 |
| 1 | 2 | 1 | 3 | 1 | 2 | 3 | 2 | 1 | 1 | 3 | 1 | 3 | 1 | 1 | 1 | 2 | 2 | 3 | 2 | 3 | 1 | 1 | 1 |
| 1 | 3 | 1 | 2 | 2 | 1 | 1 | 3 | 1 | 3 | 1 | 1 | 3 | 2 | 2 | 1 | 1 | 2 | 1 | 3 | 1 | 3 | 2 | 1 |
| 3 | 1 | 1 | 3 | 2 | 1 | 1 | 1 | 2 | 2 | 3 | 2 | 3 | 1 | 1 | 2 | 3 | 1 | 1 | 1 | 3 | 1 | 1 | 1 |
| 1 | 1 | 2 | 3 | 2 | 1 | 1 | 3 | 1 | 1 | 1 | 3 | 1 | 1 | 3 | 1 | 2 | 2 | 3 | 2 | 2 | 3 | 2 | 1 |
| 2 | 2 | 2 | 3 | 1 | 2 | 2 | 2 | 1 | 2 | 3 | 2 | 3 | 2 | 2 | 1 | 2 | 3 | 2 | 2 | 3 | 1 | 3 | 2 |
| 3 | 2 | 1 | 2 | 2 | 3 | 1 | 3 | 1 | 1 | 1 | 2 | 2 | 2 | 3 | 1 | 1 | 3 | 1 | 1 | 2 | 3 | 1 | 1 |
| 3 | 1 | 1 | 2 | 2 | 3 | 2 | 1 | 2 | 3 | 1 | 1 | 1 | 2 | 3 | 1 | 1 | 2 | 2 | 3 | 2 | 1 | 1 | 3 |
| 2 | 1 | 2 | 2 | 3 | 2 | 1 | 3 | 1 | 1 | 3 | 2 | 1 | 1 | 1 | 3 | 2 | 2 | 1 | 3 | 1 | 1 | 3 | 2 |
| 2 | 2 | 2 | 1 | 2 | 3 | 2 | 1 | 1 | 2 | 3 | 1 | 2 | 1 | 1 | 3 | 2 | 3 | 2 | 1 | 3 | 2 | 2 | 3 |
| 1 | 2 | 1 | 2 | 1 | 3 | 2 | 2 | 3 | 1 | 1 | 1 | 2 | 2 | 3 | 2 | 3 | 1 | 2 | 1 | 3 | 2 | 3 | 2 |
| 1 | 2 | 1 | 1 | 3 | 1 | 1 | 1 | 2 | 2 | 1 | 3 | 1 | 3 | 1 | 3 | 2 | 2 | 3 | 2 | 1 | 1 | 1 | 3 |
| 3 | 1 | 1 | 2 | 2 | 3 | 2 | 3 | 1 | 1 | 1 | 2 | 3 | 2 | 3 | 1 | 2 | 2 | 3 | 1 | 2 | 1 | 2 | 1 |
| 1 | 1 | 1 | 2 | 1 | 1 | 3 | 2 | 1 | 3 | 2 | 2 | 2 | 1 | 1 | 2 | 3 | 1 | 3 | 1 | 3 | 1 | 1 | 3 |
| 3 | 1 | 2 | 2 | 1 | 1 | 1 | 3 | 1 | 1 | 3 | 2 | 1 | 1 | 3 | 2 | 3 | 1 | 1 | 2 | 3 | 2 | 2 | 2 |
| 2 | 1 | 2 | 3 | 2 | 3 | 2 | 3 | 2 | 2 | 3 | 2 | 2 | 2 | 1 | 3 | 2 | 3 | 2 | 2 | 1 | 2 | 2 | 1 |
| 3 | 1 | 3 | 2 | 2 | 1 | 2 | 1 | 2 | 3 | 2 | 1 | 3 | 2 | 2 | 1 | 3 | 1 | 3 | 2 | 2 | 1 | 2 | 1 |
| 3 | 1 | 1 | 1 | 3 | 1 | 1 | 1 | 3 | 1 | 1 | 3 | 2 | 3 | 2 | 2 | 1 | 1 | 3 | 2 | 2 | 1 | 1 | 1 |
| 2 | 1 | 3 | 2 | 1 | 2 | 2 | 1 | 3 | 2 | 1 | 1 | 3 | 2 | 1 | 2 | 3 | 2 | 3 | 1 | 2 | 2 | 3 | 2 |
| 2 | 2 | 3 | 2 | 3 | 2 | 3 | 1 | 2 | 2 | 3 | 1 | 1 | 2 | 1 | 2 | 2 | 3 | 2 | 3 | 1 | 1 | 1 | 2 |
| 1 | 2 | 3 | 2 | 3 | 1 | 1 | 1 | 3 | 1 | 3 | 2 | 2 | 1 | 1 | 3 | 2 | 3 | 1 | 2 | 2 | 1 | 1 | 1 |
| 3 | 1 | 2 | 2 | 3 | 1 | 1 | 2 | 3 | 1 | 2 | 2 | 3 | 1 | 3 | 1 | 2 | 1 | 2 | 3 | 2 | 1 | 1 | 1 |
| 1 | 1 | 3 | 1 | 2 | 3 | 1 | 2 | 1 | 3 | 2 | 2 | 1 | 1 | 3 | 2 | 3 | 2 | 1 | 1 | 3 | 2 | 2 | 1 |
| 2 | 1 | 3 | 2 | 2 | 3 | 2 | 2 | 1 | 2 | 2 | 3 | 1 | 3 | 1 | 1 | 2 | 2 | 2 | 1 | 3 | 1 | 1 | 3 |
| 2 | 2 | 2 | 1 | 2 | 1 | 3 | 2 | 3 | 1 | 1 | 2 | 2 | 1 | 2 | 3 | 1 | 3 | 2 | 3 | 1 | 1 | 1 | 3 |
| 3 | 1 | 2 | 1 | 3 | 1 | 2 | 2 | 2 | 1 | 3 | 1 | 1 | 2 | 3 | 1 | 1 | 2 | 2 | 1 | 1 | 3 | 2 | 3 |
| 2 | 2 | 2 | 3 | 1 | 1 | 3 | 1 | 1 | 3 | 1 | 3 | 1 | 2 | 2 | 2 | 3 | 1 | 1 | 1 | 2 | 2 | 3 | 1 |
| 1 | 2 | 3 | 1 | 1 | 2 | 1 | 1 | 3 | 1 | 3 | 2 | 2 | 3 | 1 | 2 | 1 | 1 | 1 | 2 | 3 | 2 | 3 | 1 |
| 2 | 3 | 2 | 2 | 2 | 1 | 2 | 3 | 2 | 1 | 3 | 2 | 3 | 2 | 1 | 3 | 1 | 2 | 2 | 3 | 1 | 1 | 2 | 2 |
| 2 | 2 | 2 | 1 | 1 | 3 | 2 | 3 | 1 | 3 | 2 | 2 | 1 | 2 | 1 | 3 | 1 | 1 | 3 | 2 | 1 | 3 | 2 | 1 |
| 3 | 1 | 2 | 2 | 2 | 1 | 2 | 3 | 2 | 3 | 2 | 2 | 2 | 3 | 1 | 1 | 3 | 2 | 2 | 1 | 1 | 3 | 1 | 2 |
| 2 | 1 | 3 | 2 | 2 | 1 | 3 | 1 | 3 | 1 | 1 | 1 | 3 | 2 | 3 | 1 | 2 | 1 | 1 | 1 | 3 | 2 | 2 | 1 |
| 3 | 2 | 1 | 1 | 2 | 3 | 1 | 2 | 1 | 1 | 2 | 3 | 1 | 1 | 3 | 2 | 3 | 2 | 1 | 2 | 1 | 2 | 1 | 3 |
| 1 | 1 | 2 | 3 | 1 | 1 | 3 | 2 | 3 | 2 | 2 | 1 | 3 | 2 | 1 | 2 | 1 | 3 | 1 | 2 | 1 | 3 | 2 | 1 |
| 2 | 1 | 1 | 1 | 2 | 2 | 3 | 1 | 3 | 2 | 2 | 2 | 3 | 2 | 2 | 2 | 3 | 1 | 2 | 2 | 3 | 2 | 1 | 3 |
| 2 | 1 | 1 | 2 | 3 | 1 | 1 | 3 | 1 | 1 | 2 | 1 | 1 | 3 | 2 | 1 | 2 | 3 | 1 | 3 | 2 | 3 | 2 | 2 |
| 1 | 1 | 1 | 2 | 3 | 2 | 1 | 1 | 2 | 1 | 3 | 2 | 3 | 2 | 2 | 3 | 2 | 2 | 1 | 3 | 2 | 2 | 1 | 3 |
| 1 | 3 | 1 | 3 | 2 | 2 | 1 | 3 | 2 | 3 | 1 | 1 | 1 | 2 | 3 | 2 | 2 | 3 | 2 | 2 | 1 | 1 | 1 | 2 |
| 3 | 1 | 1 | 1 | 2 | 1 | 3 | 1 | 1 | 1 | 2 | 3 | 2 | 1 | 2 | 2 | 3 | 2 | 2 | 2 | 3 | 2 | 3 | 1 |
| 1 | 3 | 2 | 2 | 1 | 2 | 1 | 1 | 3 | 2 | 2 | 2 | 3 | 2 | 3 | 1 | 3 | 1 | 1 | 2 | 2 | 1 | 1 | 3 |
| 3 | 1 | 3 | 2 | 2 | 2 | 1 | 2 | 1 | 3 | 2 | 2 | 1 | 3 | 1 | 1 | 2 | 1 | 2 | 3 | 2 | 2 | 3 | 2 |
| 1 | 3 | 1 | 3 | 2 | 2 | 1 | 2 | 2 | 1 | 3 | 1 | 1 | 3 | 1 | 1 | 3 | 1 | 2 | 2 | 2 | 1 | 1 | 3 |
| 3 | 1 | 3 | 2 | 2 | 1 | 1 | 2 | 3 | 1 | 1 | 1 | 2 | 1 | 1 | 3 | 2 | 1 | 2 | 2 | 2 | 3 | 2 | 3 |
| 1 | 2 | 3 | 1 | 2 | 3 | 1 | 1 | 2 | 1 | 3 | 2 | 2 | 3 | 1 | 1 | 3 | 2 | 1 | 2 | 1 | 2 | 1 | 3 |
| 1 | 2 | 1 | 3 | 1 | 2 | 1 | 2 | 3 | 1 | 3 | 1 | 2 | 3 | 1 | 1 | 1 | 3 | 2 | 2 | 1 | 3 | 2 | 1 |
| 2 | 1 | 2 | 3 | 2 | 1 | 1 | 1 | 3 | 1 | 1 | 1 | 3 | 2 | 3 | 1 | 1 | 1 | 3 | 1 | 1 | 3 | 1 | 1 |
| 2 | 3 | 1 | 1 | 2 | 3 | 2 | 1 | 3 | 1 | 1 | 1 | 2 | 3 | 1 | 1 | 2 | 3 | 2 | 2 | 3 | 1 | 1 | 1 |
| 1 | 1 | 2 | 2 | 3 | 1 | 1 | 2 | 1 | 3 | 2 | 3 | 2 | 3 | 2 | 3 | 1 | 3 | 2 | 2 | 2 | 1 | 1 | 2 |
| 1 | 3 | 1 | 2 | 1 | 2 | 2 | 3 | 2 | 2 | 2 | 3 | 1 | 2 | 2 | 1 | 1 | 2 | 3 | 1 | 1 | 3 | 1 | 3 |
| 1 | 1 | 1 | 3 | 2 | 2 | 3 | 2 | 1 | 1 | 1 | 3 | 2 | 2 | 3 | 1 | 1 | 3 | 1 | 2 | 1 | 1 | 1 | 3 |
| 3 | 2 | 2 | 1 | 1 | 3 | 1 | 3 | 1 | 2 | 2 | 1 | 2 | 3 | 1 | 3 | 1 | 2 | 3 | 2 | 1 | 2 | 2 | 1 |
| 1 | 3 | 1 | 1 | 3 | 1 | 2 | 1 | 2 | 1 | 1 | 3 | 1 | 1 | 3 | 1 | 2 | 2 | 3 | 1 | 1 | 2 | 2 | 3 |
| 3 | 2 | 1 | 3 | 1 | 1 | 1 | 2 | 2 | 2 | 3 | 1 | 1 | 2 | 2 | 3 | 1 | 2 | 3 | 2 | 3 | 1 | 1 | 1 |
| 1 | 1 | 3 | 1 | 3 | 2 | 1 | 3 | 1 | 2 | 2 | 3 | 1 | 2 | 1 | 1 | 3 | 2 | 1 | 2 | 1 | 2 | 3 | 1 |
| 2 | 3 | 1 | 2 | 1 | 2 | 1 | 3 | 2 | 1 | 3 | 2 | 3 | 1 | 1 | 3 | 1 | 1 | 1 | 2 | 1 | 1 | 3 | 2 |
| 1 | 3 | 1 | 2 | 1 | 1 | 2 | 3 | 1 | 2 | 3 | 1 | 3 | 1 | 1 | 1 | 2 | 3 | 1 | 1 | 3 | 1 | 2 | 1 |
| 1 | 2 | 3 | 2 | 3 | 1 | 1 | 1 | 3 | 2 | 1 | 2 | 2 | 2 | 3 | 2 | 3 | 1 | 2 | 1 | 2 | 1 | 3 | 2 |
| 1 | 1 | 2 | 1 | 1 | 3 | 1 | 3 | 1 | 1 | 2 | 2 | 3 | 1 | 2 | 1 | 2 | 3 | 1 | 1 | 3 | 1 | 2 | 3 |
| 2 | 1 | 1 | 3 | 2 | 3 | 2 | 1 | 2 | 2 | 2 | 1 | 3 | 2 | 1 | 3 | 1 | 1 | 2 | 3 | 1 | 1 | 3 | 2 |
| 2 | 1 | 2 | 3 | 2 | 2 | 1 | 3 | 1 | 2 | 2 | 2 | 3 | 2 | 2 | 3 | 1 | 3 | 1 | 2 | 2 | 3 | 1 | 2 |
| 1 | 3 | 2 | 2 | 2 | 3 | 2 | 1 | 2 | 3 | 1 | 1 | 3 | 1 | 3 | 1 | 2 | 1 | 3 | 2 | 1 | 2 | 2 | 2 |
| 3 | 1 | 3 | 1 | 1 | 1 | 2 | 3 | 2 | 2 | 1 | 2 | 3 | 2 | 1 | 2 | 2 | 2 | 1 | 3 | 2 | 1 | 3 | 2 |
| 2 | 1 | 2 | 3 | 2 | 3 | 1 | 3 | 1 | 1 | 2 | 3 | 2 | 3 | 2 | 2 | 2 | 3 | 1 | 2 | 2 | 2 | 1 | 1 |
| 3 | 2 | 1 | 2 | 3 | 2 | 2 | 2 | 3 | 2 | 2 | 2 | 1 | 2 | 1 | 3 | 1 | 1 | 2 | 3 | 2 | 1 | 2 | 3 |
| 3 | 1 | 3 | 2 | 1 | 2 | 1 | 2 | 1 | 3 | 1 | 1 | 3 | 1 | 1 | 1 | 3 | 1 | 1 | 1 | 2 | 2 | 2 | 3 |
| 1 | 2 | 3 | 1 | 3 | 2 | 3 | 1 | 1 | 3 | 2 | 1 | 1 | 1 | 2 | 3 | 2 | 1 | 3 | 2 | 2 | 1 | 2 | 2 |
| 2 | 2 | 1 | 1 | 3 | 1 | 1 | 3 | 2 | 3 | 1 | 3 | 2 | 2 | 1 | 2 | 2 | 3 | 2 | 3 | 1 | 2 | 1 | 2 |
| 1 | 2 | 3 | 1 | 1 | 1 | 2 | 3 | 1 | 3 | 1 | 1 | 2 | 1 | 2 | 2 | 3 | 2 | 2 | 3 | 2 | 2 | 2 | 3 |
| 3 | 1 | 2 | 2 | 1 | 1 | 2 | 3 | 1 | 2 | 2 | 1 | 2 | 3 | 2 | 3 | 1 | 1 | 2 | 2 | 3 | 1 | 2 | 3 |
| 3 | 1 | 1 | 1 | 2 | 3 | 2 | 2 | 1 | 1 | 1 | 3 | 1 | 2 | 1 | 2 | 3 | 1 | 1 | 1 | 3 | 2 | 1 | 3 |
| 2 | 1 | 2 | 2 | 3 | 2 | 2 | 3 | 1 | 2 | 2 | 2 | 3 | 1 | 2 | 1 | 2 | 2 | 1 | 3 | 2 | 3 | 2 | 3 |
| 2 | 2 | 2 | 1 | 2 | 3 | 2 | 2 | 2 | 3 | 2 | 3 | 2 | 1 | 2 | 3 | 2 | 1 | 1 | 3 | 2 | 1 | 3 | 2 |
| 1 | 1 | 2 | 2 | 3 | 1 | 1 | 1 | 3 | 1 | 1 | 2 | 2 | 3 | 2 | 3 | 2 | 3 | 1 | 1 | 2 | 2 | 3 | 1 |
| 2 | 3 | 1 | 3 | 2 | 2 | 2 | 3 | 1 | 1 | 2 | 2 | 2 | 3 | 2 | 2 | 2 | 3 | 1 | 3 | 2 | 1 | 1 | 2 |
| 3 | 1 | 2 | 3 | 2 | 1 | 2 | 1 | 1 | 2 | 3 | 1 | 2 | 3 | 2 | 3 | 2 | 3 | 2 | 1 | 1 | 1 | 2 | 2 |
| 1 | 2 | 3 | 2 | 3 | 1 | 3 | 1 | 3 | 1 | 1 | 3 | 1 | 1 | 2 | 2 | 2 | 3 | 2 | 2 | 2 | 1 | 2 | 2 |
| 3 | 2 | 3 | 1 | 2 | 1 | 1 | 1 | 3 | 2 | 1 | 2 | 2 | 3 | 2 | 2 | 3 | 1 | 2 | 1 | 3 | 1 | 1 | 1 |
| 3 | 1 | 1 | 3 | 2 | 1 | 3 | 1 | 1 | 2 | 1 | 3 | 1 | 1 | 1 | 3 | 2 | 2 | 1 | 1 | 2 | 1 | 3 | 1 |
| 2 | 2 | 3 | 2 | 3 | 2 | 1 | 3 | 2 | 2 | 1 | 1 | 3 | 1 | 3 | 2 | 2 | 3 | 2 | 2 | 2 | 1 | 1 | 2 |
| 2 | 1 | 3 | 2 | 1 | 3 | 2 | 1 | 1 | 3 | 2 | 2 | 3 | 2 | 2 | 1 | 3 | 1 | 1 | 2 | 1 | 3 | 2 | 2 |
| 1 | 1 | 2 | 2 | 2 | 3 | 1 | 1 | 3 | 2 | 1 | 2 | 1 | 1 | 2 | 3 | 1 | 1 | 2 | 3 | 2 | 3 | 2 | 3 |
| 2 | 1 | 3 | 1 | 1 | 1 | 2 | 2 | 3 | 2 | 1 | 3 | 2 | 1 | 2 | 2 | 2 | 3 | 1 | 3 | 1 | 3 | 1 | 1 |
| 2 | 3 | 2 | 1 | 2 | 1 | 2 | 3 | 2 | 2 | 1 | 1 | 2 | 3 | 1 | 3 | 1 | 2 | 3 | 2 | 2 | 3 | 2 | 1 |
| 2 | 1 | 2 | 2 | 2 | 3 | 1 | 2 | 1 | 1 | 3 | 1 | 3 | 1 | 1 | 2 | 3 | 1 | 1 | 3 | 1 | 1 | 3 | 2 |
| 2 | 2 | 3 | 1 | 1 | 2 | 1 | 3 | 2 | 3 | 2 | 1 | 1 | 2 | 3 | 1 | 1 | 2 | 1 | 2 | 3 | 1 | 2 | 3 |
| 3 | 2 | 1 | 3 | 2 | 2 | 2 | 3 | 2 | 3 | 1 | 1 | 2 | 1 | 3 | 1 | 1 | 2 | 2 | 1 | 3 | 2 | 2 | 2 |
| 1 | 1 | 1 | 3 | 1 | 2 | 3 | 1 | 2 | 2 | 3 | 2 | 1 | 1 | 2 | 2 | 2 | 3 | 2 | 3 | 2 | 3 | 1 | 1 |
| 3 | 1 | 1 | 3 | 1 | 2 | 2 | 3 | 2 | 2 | 3 | 1 | 3 | 2 | 2 | 1 | 1 | 2 | 1 | 3 | 1 | 2 | 1 | 1 |
| 1 | 3 | 1 | 2 | 2 | 1 | 2 | 3 | 2 | 1 | 3 | 2 | 3 | 1 | 2 | 3 | 2 | 1 | 1 | 1 | 2 | 3 | 2 | 2 |
| 3 | 1 | 1 | 2 | 2 | 2 | 1 | 3 | 1 | 2 | 3 | 2 | 1 | 3 | 1 | 2 | 1 | 2 | 3 | 1 | 1 | 2 | 3 | 2 |
| 3 | 1 | 2 | 1 | 3 | 1 | 1 | 3 | 2 | 3 | 2 | 1 | 2 | 2 | 1 | 1 | 3 | 2 | 1 | 1 | 3 | 2 | 2 | 1 |
| 2 | 1 | 2 | 3 | 1 | 1 | 2 | 2 | 1 | 2 | 3 | 1 | 3 | 1 | 1 | 3 | 1 | 1 | 2 | 1 | 3 | 1 | 3 | 2 |
| 2 | 2 | 2 | 3 | 2 | 2 | 1 | 2 | 3 | 1 | 1 | 3 | 2 | 3 | 1 | 2 | 2 | 2 | 3 | 2 | 2 | 2 | 3 | 2 |
| 3 | 2 | 1 | 1 | 1 | 3 | 1 | 2 | 2 | 3 | 2 | 3 | 2 | 2 | 1 | 2 | 1 | 2 | 3 | 1 | 1 | 1 | 2 | 3 |
| 2 | 2 | 3 | 2 | 3 | 1 | 2 | 1 | 3 | 2 | 1 | 3 | 2 | 2 | 1 | 3 | 1 | 2 | 1 | 2 | 2 | 2 | 3 | 2 |
| 3 | 1 | 1 | 2 | 2 | 1 | 1 | 3 | 1 | 2 | 1 | 1 | 1 | 3 | 1 | 1 | 3 | 1 | 3 | 1 | 1 | 3 | 2 | 1 |
| 3 | 1 | 2 | 2 | 3 | 2 | 1 | 3 | 1 | 1 | 2 | 3 | 1 | 1 | 2 | 2 | 2 | 3 | 2 | 1 | 3 | 2 | 1 | 2 |
| 1 | 1 | 1 | 2 | 1 | 1 | 3 | 1 | 3 | 1 | 3 | 1 | 3 | 1 | 1 | 2 | 3 | 1 | 2 | 2 | 2 | 1 | 3 | 2 |
| 1 | 1 | 2 | 2 | 1 | 2 | 3 | 2 | 3 | 1 | 1 | 2 | 1 | 3 | 1 | 2 | 2 | 3 | 2 | 2 | 3 | 1 | 1 | 3 |
| 2 | 2 | 1 | 1 | 3 | 1 | 2 | 2 | 2 | 1 | 2 | 3 | 2 | 3 | 1 | 2 | 1 | 3 | 2 | 1 | 3 | 1 | 3 | 2 |
| 2 | 2 | 1 | 1 | 1 | 3 | 1 | 2 | 1 | 3 | 2 | 3 | 2 | 2 | 2 | 3 | 2 | 2 | 3 | 2 | 3 | 2 | 2 | 1 |
| 2 | 1 | 2 | 2 | 3 | 1 | 2 | 2 | 2 | 1 | 2 | 3 | 1 | 1 | 3 | 1 | 3 | 2 | 1 | 2 | 1 | 3 | 2 | 3 |
| 1 | 1 | 1 | 2 | 2 | 2 | 3 | 1 | 2 | 3 | 1 | 3 | 2 | 1 | 3 | 2 | 2 | 2 | 1 | 1 | 3 | 1 | 3 | 1 |
| 1 | 2 | 1 | 1 | 1 | 3 | 2 | 2 | 3 | 2 | 2 | 2 | 3 | 1 | 2 | 3 | 2 | 2 | 2 | 3 | 1 | 1 | 2 | 3 |
| 3 | 1 | 2 | 2 | 3 | 2 | 3 | 1 | 2 | 3 | 1 | 1 | 2 | 1 | 1 | 2 | 3 | 2 | 2 | 1 | 2 | 2 | 3 | 1 |
| 3 | 1 | 2 | 3 | 1 | 1 | 3 | 1 | 1 | 1 | 2 | 1 | 2 | 3 | 1 | 2 | 1 | 2 | 3 | 1 | 1 | 2 | 1 | 3 |
| 2 | 2 | 1 | 1 | 1 | 3 | 2 | 2 | 1 | 2 | 2 | 3 | 1 | 1 | 3 | 2 | 3 | 1 | 1 | 3 | 2 | 2 | 3 | 1 |
| 2 | 2 | 3 | 2 | 1 | 1 | 3 | 1 | 1 | 1 | 2 | 1 | 3 | 1 | 3 | 1 | 2 | 2 | 2 | 3 | 2 | 3 | 2 | 2 |
| 3 | 1 | 3 | 1 | 2 | 2 | 3 | 1 | 3 | 2 | 2 | 2 | 1 | 1 | 3 | 2 | 1 | 2 | 2 | 1 | 3 | 1 | 2 | 2 |
| 1 | 3 | 2 | 3 | 1 | 2 | 1 | 1 | 2 | 1 | 3 | 1 | 1 | 2 | 3 | 1 | 2 | 1 | 1 | 1 | 2 | 3 | 2 | 3 |
| 3 | 1 | 2 | 1 | 1 | 2 | 1 | 3 | 2 | 3 | 1 | 1 | 2 | 2 | 2 | 3 | 1 | 3 | 2 | 2 | 3 | 2 | 1 | 2 |
| 1 | 3 | 1 | 2 | 1 | 2 | 2 | 2 | 3 | 2 | 1 | 3 | 2 | 1 | 3 | 1 | 1 | 1 | 3 | 2 | 1 | 2 | 3 | 2 |
| 3 | 2 | 2 | 1 | 2 | 3 | 1 | 1 | 2 | 3 | 2 | 2 | 3 | 1 | 1 | 2 | 2 | 2 | 3 | 1 | 1 | 2 | 3 | 2 |
| 1 | 2 | 3 | 1 | 1 | 1 | 3 | 1 | 2 | 2 | 2 | 1 | 3 | 2 | 2 | 3 | 2 | 3 | 1 | 3 | 1 | 2 | 1 | 2 |
| 1 | 1 | 1 | 2 | 1 | 3 | 1 | 3 | 1 | 1 | 3 | 2 | 2 | 1 | 2 | 3 | 1 | 2 | 3 | 2 | 3 | 1 | 2 | 1 |
| 2 | 2 | 1 | 3 | 2 | 3 | 1 | 3 | 1 | 1 | 1 | 2 | 3 | 2 | 2 | 2 | 1 | 1 | 2 | 3 | 2 | 3 | 1 | 2 |
| 2 | 3 | 1 | 1 | 3 | 1 | 1 | 2 | 1 | 2 | 3 | 2 | 3 | 1 | 1 | 1 | 2 | 2 | 1 | 3 | 2 | 2 | 2 | 3 |
| 3 | 2 | 2 | 2 | 3 | 1 | 2 | 1 | 3 | 2 | 2 | 2 | 1 | 1 | 2 | 3 | 1 | 3 | 2 | 1 | 2 | 2 | 3 | 1 |
| 3 | 2 | 2 | 3 | 2 | 1 | 1 | 3 | 2 | 1 | 1 | 2 | 3 | 1 | 2 | 1 | 1 | 1 | 3 | 2 | 1 | 2 | 3 | 1 |
| 2 | 1 | 1 | 3 | 1 | 3 | 2 | 1 | 3 | 2 | 1 | 1 | 2 | 2 | 3 | 2 | 2 | 3 | 2 | 2 | 2 | 1 | 3 | 1 |
| 2 | 2 | 2 | 3 | 1 | 3 | 1 | 3 | 1 | 3 | 2 | 1 | 2 | 3 | 2 | 1 | 2 | 3 | 1 | 2 | 2 | 1 | 2 | 2 |
| 1 | 2 | 2 | 3 | 1 | 2 | 2 | 3 | 2 | 3 | 1 | 1 | 2 | 2 | 1 | 3 | 1 | 2 | 1 | 3 | 1 | 1 | 3 | 1 |
| 3 | 1 | 2 | 2 | 1 | 3 | 2 | 1 | 2 | 2 | 2 | 1 | 3 | 2 | 1 | 3 | 2 | 1 | 1 | 2 | 1 | 3 | 1 | 3 |
| 2 | 1 | 2 | 3 | 2 | 1 | 2 | 2 | 1 | 3 | 1 | 3 | 1 | 2 | 1 | 2 | 2 | 3 | 1 | 1 | 1 | 3 | 2 | 3 |
| 2 | 1 | 2 | 3 | 2 | 3 | 1 | 1 | 1 | 3 | 2 | 1 | 1 | 2 | 3 | 1 | 2 | 1 | 1 | 1 | 2 | 3 | 1 | 3 |
| 3 | 2 | 1 | 1 | 2 | 2 | 1 | 3 | 2 | 1 | 1 | 2 | 3 | 1 | 2 | 2 | 2 | 3 | 1 | 1 | 2 | 3 | 1 | 3 |
| 3 | 2 | 2 | 2 | 1 | 2 | 2 | 3 | 2 | 1 | 1 | 1 | 3 | 1 | 2 | 3 | 2 | 1 | 1 | 3 | 2 | 3 | 1 | 1 |
| 2 | 1 | 3 | 2 | 1 | 3 | 1 | 1 | 2 | 2 | 3 | 2 | 2 | 3 | 2 | 2 | 1 | 1 | 1 | 3 | 1 | 1 | 2 | 3 |
| 2 | 1 | 2 | 2 | 3 | 2 | 2 | 1 | 3 | 2 | 2 | 1 | 2 | 3 | 2 | 1 | 3 | 2 | 3 | 2 | 3 | 2 | 1 | 1 |
| 3 | 1 | 3 | 2 | 3 | 1 | 1 | 1 | 3 | 2 | 2 | 1 | 2 | 1 | 2 | 3 | 1 | 1 | 1 | 3 | 2 | 1 | 2 | 1 |
| 1 | 2 | 1 | 2 | 1 | 3 | 1 | 1 | 3 | 2 | 2 | 3 | 1 | 2 | 3 | 1 | 3 | 2 | 2 | 2 | 1 | 2 | 3 | 1 |
| 2 | 2 | 2 | 1 | 3 | 1 | 1 | 3 | 2 | 1 | 1 | 3 | 1 | 1 | 2 | 1 | 1 | 3 | 2 | 3 | 1 | 3 | 2 | 1 |
| 2 | 3 | 2 | 3 | 2 | 1 | 2 | 1 | 1 | 3 | 1 | 2 | 1 | 2 | 2 | 2 | 3 | 2 | 1 | 1 | 3 | 1 | 1 | 3 |
| 2 | 1 | 3 | 1 | 1 | 3 | 1 | 3 | 2 | 2 | 3 | 2 | 1 | 2 | 2 | 3 | 2 | 2 | 1 | 2 | 1 | 1 | 3 | 2 |
| 3 | 2 | 3 | 2 | 2 | 1 | 2 | 2 | 1 | 3 | 2 | 2 | 2 | 1 | 1 | 3 | 2 | 2 | 1 | 3 | 1 | 3 | 2 | 1 |
| 1 | 1 | 2 | 1 | 2 | 1 | 3 | 2 | 3 | 1 | 2 | 3 | 2 | 3 | 1 | 1 | 1 | 2 | 2 | 3 | 1 | 1 | 2 | 3 |
| 2 | 2 | 1 | 3 | 1 | 3 | 1 | 1 | 2 | 1 | 3 | 1 | 3 | 2 | 3 | 1 | 2 | 2 | 1 | 2 | 1 | 3 | 2 | 2 |
| 3 | 1 | 1 | 3 | 2 | 3 | 1 | 3 | 2 | 2 | 1 | 1 | 2 | 3 | 1 | 2 | 2 | 2 | 3 | 2 | 1 | 1 | 1 | 2 |
| 1 | 1 | 2 | 3 | 2 | 1 | 1 | 1 | 3 | 2 | 1 | 1 | 1 | 3 | 1 | 1 | 1 | 3 | 2 | 3 | 1 | 2 | 3 | 1 |
| 3 | 2 | 2 | 1 | 3 | 2 | 2 | 1 | 2 | 3 | 1 | 2 | 3 | 1 | 1 | 2 | 1 | 2 | 2 | 3 | 2 | 3 | 2 | 1 |
| 1 | 1 | 1 | 2 | 3 | 1 | 3 | 2 | 2 | 1 | 3 | 1 | 3 | 2 | 1 | 3 | 1 | 1 | 2 | 2 | 1 | 2 | 3 | 2 |
| 3 | 1 | 2 | 1 | 2 | 1 | 3 | 1 | 1 | 3 | 1 | 2 | 2 | 1 | 3 | 2 | 2 | 1 | 3 | 2 | 3 | 1 | 2 | 1 |
| 1 | 2 | 1 | 3 | 2 | 2 | 2 | 3 | 2 | 2 | 3 | 1 | 3 | 1 | 2 | 2 | 2 | 1 | 2 | 3 | 1 | 3 | 2 | 1 |
| 2 | 1 | 3 | 1 | 1 | 2 | 1 | 3 | 2 | 2 | 1 | 3 | 2 | 1 | 3 | 2 | 1 | 1 | 3 | 1 | 3 | 2 | 1 | 2 |
| 3 | 1 | 1 | 2 | 2 | 2 | 3 | 2 | 1 | 2 | 2 | 3 | 2 | 3 | 1 | 1 | 3 | 2 | 2 | 2 | 1 | 3 | 2 | 1 |
| 3 | 2 | 1 | 3 | 2 | 1 | 1 | 3 | 1 | 1 | 3 | 1 | 3 | 1 | 1 | 2 | 2 | 1 | 3 | 1 | 2 | 2 | 1 | 1 |
| 1 | 1 | 2 | 3 | 2 | 3 | 2 | 2 | 1 | 2 | 3 | 2 | 1 | 2 | 3 | 2 | 1 | 1 | 1 | 2 | 1 | 3 | 2 | 3 |
| 3 | 1 | 1 | 2 | 2 | 1 | 3 | 2 | 2 | 1 | 3 | 1 | 3 | 2 | 1 | 1 | 1 | 2 | 2 | 3 | 2 | 2 | 2 | 3 |
| 3 | 1 | 1 | 1 | 2 | 2 | 3 | 1 | 1 | 3 | 1 | 2 | 1 | 3 | 2 | 1 | 1 | 3 | 1 | 1 | 1 | 2 | 3 | 1 |
| 3 | 2 | 3 | 2 | 1 | 2 | 2 | 1 | 2 | 3 | 2 | 3 | 1 | 2 | 2 | 2 | 1 | 2 | 3 | 1 | 2 | 1 | 3 | 1 |
| 2 | 1 | 2 | 2 | 1 | 2 | 3 | 1 | 3 | 1 | 1 | 1 | 3 | 2 | 2 | 3 | 1 | 1 | 2 | 1 | 3 | 2 | 1 | 3 |
| 2 | 1 | 2 | 3 | 2 | 1 | 2 | 2 | 3 | 2 | 1 | 2 | 2 | 3 | 1 | 3 | 2 | 1 | 3 | 1 | 2 | 3 | 1 | 1 |
| 3 | 2 | 3 | 1 | 2 | 2 | 3 | 1 | 1 | 2 | 1 | 3 | 2 | 1 | 3 | 1 | 2 | 2 | 3 | 2 | 2 | 2 | 1 | 1 |
| 1 | 3 | 2 | 1 | 1 | 3 | 2 | 2 | 3 | 2 | 2 | 2 | 3 | 1 | 2 | 2 | 3 | 1 | 1 | 1 | 2 | 2 | 2 | 3 |
| 3 | 1 | 1 | 3 | 2 | 2 | 2 | 3 | 1 | 2 | 2 | 2 | 1 | 1 | 3 | 2 | 2 | 2 | 1 | 1 | 3 | 1 | 1 | 3 |
| 3 | 1 | 3 | 1 | 1 | 3 | 1 | 2 | 1 | 1 | 1 | 2 | 3 | 1 | 2 | 1 | 2 | 2 | 3 | 2 | 2 | 1 | 2 | 3 |
| 1 | 2 | 3 | 1 | 2 | 3 | 1 | 3 | 2 | 2 | 3 | 2 | 2 | 1 | 1 | 2 | 1 | 3 | 2 | 2 | 1 | 3 | 2 | 2 |
| 2 | 1 | 2 | 3 | 1 | 2 | 1 | 2 | 2 | 2 | 3 | 1 | 1 | 3 | 1 | 3 | 2 | 3 | 2 | 2 | 1 | 1 | 3 | 1 |
| 3 | 1 | 3 | 1 | 2 | 3 | 1 | 2 | 2 | 1 | 1 | 1 | 3 | 2 | 3 | 1 | 2 | 2 | 2 | 1 | 2 | 3 | 1 | 1 |
| 1 | 2 | 1 | 3 | 2 | 2 | 1 | 1 | 3 | 1 | 3 | 2 | 3 | 1 | 2 | 3 | 1 | 3 | 1 | 1 | 2 | 1 | 1 | 1 |
| 2 | 2 | 2 | 1 | 2 | 2 | 3 | 2 | 2 | 1 | 3 | 1 | 2 | 1 | 1 | 1 | 3 | 1 | 3 | 2 | 2 | 3 | 1 | 3 |
| 1 | 3 | 1 | 1 | 2 | 1 | 2 | 2 | 3 | 1 | 2 | 1 | 3 | 2 | 2 | 3 | 1 | 1 | 3 | 2 | 2 | 3 | 1 | 1 |
| 2 | 1 | 3 | 2 | 3 | 2 | 1 | 1 | 1 | 3 | 2 | 3 | 2 | 1 | 3 | 1 | 2 | 2 | 3 | 2 | 1 | 1 | 1 | 2 |
| 1 | 3 | 2 | 1 | 3 | 2 | 3 | 1 | 2 | 1 | 2 | 3 | 1 | 2 | 2 | 2 | 3 | 1 | 1 | 2 | 1 | 2 | 2 | 3 |
| 2 | 3 | 2 | 1 | 2 | 2 | 3 | 1 | 1 | 2 | 2 | 1 | 3 | 1 | 1 | 2 | 1 | 3 | 2 | 3 | 1 | 3 | 1 | 1 |
| 2 | 3 | 1 | 2 | 1 | 2 | 3 | 1 | 3 | 1 | 2 | 1 | 3 | 1 | 1 | 3 | 2 | 2 | 2 | 1 | 1 | 2 | 3 | 2 |
| 3 | 1 | 1 | 3 | 1 | 1 | 3 | 2 | 1 | 1 | 3 | 2 | 1 | 2 | 1 | 1 | 1 | 3 | 2 | 1 | 1 | 1 | 2 | 3 |
| 2 | 2 | 2 | 1 | 1 | 3 | 2 | 3 | 2 | 3 | 1 | 2 | 1 | 1 | 3 | 1 | 1 | 1 | 3 | 1 | 2 | 1 | 3 | 1 |
| 2 | 1 | 2 | 2 | 3 | 2 | 2 | 3 | 1 | 1 | 2 | 3 | 2 | 3 | 2 | 2 | 2 | 1 | 1 | 1 | 3 | 1 | 3 | 1 |
| 3 | 1 | 1 | 2 | 1 | 1 | 2 | 3 | 1 | 2 | 3 | 1 | 3 | 1 | 2 | 3 | 1 | 2 | 2 | 1 | 2 | 2 | 3 | 1 |
| 2 | 1 | 3 | 1 | 3 | 1 | 1 | 1 | 3 | 1 | 3 | 1 | 3 | 1 | 1 | 2 | 2 | 3 | 2 | 1 | 2 | 2 | 1 | 1 |
| 1 | 2 | 3 | 2 | 1 | 2 | 1 | 1 | 2 | 3 | 1 | 3 | 1 | 2 | 1 | 2 | 3 | 2 | 2 | 2 | 3 | 2 | 3 | 1 |
| 1 | 1 | 2 | 1 | 3 | 1 | 2 | 1 | 1 | 3 | 1 | 2 | 2 | 3 | 1 | 2 | 2 | 3 | 2 | 3 | 2 | 2 | 2 | 3 |
| 2 | 2 | 2 | 3 | 1 | 2 | 3 | 1 | 2 | 1 | 1 | 2 | 1 | 3 | 1 | 1 | 3 | 1 | 3 | 1 | 1 | 2 | 3 | 1 |
| 1 | 3 | 1 | 2 | 3 | 1 | 1 | 2 | 1 | 1 | 3 | 2 | 2 | 3 | 2 | 3 | 1 | 1 | 2 | 3 | 2 | 2 | 2 | 1 |
| 1 | 3 | 1 | 2 | 3 | 1 | 1 | 1 | 3 | 1 | 1 | 1 | 3 | 2 | 3 | 2 | 1 | 3 | 1 | 1 | 2 | 1 | 2 | 2 |
| 2 | 3 | 2 | 2 | 1 | 1 | 1 | 2 | 3 | 2 | 1 | 2 | 3 | 2 | 1 | 3 | 2 | 1 | 1 | 2 | 2 | 3 | 1 | 3 |
| 2 | 1 | 3 | 2 | 1 | 3 | 2 | 3 | 2 | 3 | 1 | 1 | 3 | 2 | 2 | 1 | 2 | 2 | 2 | 3 | 2 | 2 | 1 | 2 |
| 1 | 3 | 2 | 3 | 1 | 1 | 2 | 3 | 2 | 2 | 2 | 3 | 2 | 1 | 1 | 1 | 3 | 1 | 3 | 2 | 2 | 2 | 1 | 1 |
| 3 | 1 | 2 | 1 | 1 | 1 | 2 | 3 | 1 | 3 | 1 | 1 | 2 | 2 | 3 | 1 | 3 | 2 | 1 | 1 | 2 | 2 | 3 | 2 |
| 2 | 3 | 1 | 2 | 3 | 1 | 3 | 1 | 1 | 1 | 2 | 2 | 3 | 2 | 2 | 2 | 1 | 1 | 3 | 2 | 3 | 2 | 2 | 2 |
| 1 | 1 | 1 | 2 | 1 | 1 | 3 | 2 | 1 | 3 | 2 | 3 | 2 | 3 | 1 | 3 | 2 | 1 | 1 | 2 | 1 | 3 | 2 | 1 |
| 2 | 1 | 2 | 3 | 1 | 1 | 1 | 2 | 1 | 2 | 3 | 2 | 3 | 1 | 2 | 1 | 3 | 2 | 1 | 1 | 3 | 1 | 3 | 1 |
| 1 | 2 | 2 | 3 | 2 | 1 | 1 | 3 | 1 | 3 | 2 | 3 | 1 | 2 | 2 | 1 | 2 | 1 | 3 | 1 | 2 | 3 | 1 | 2 |
| 1 | 3 | 1 | 3 | 2 | 1 | 1 | 3 | 1 | 1 | 2 | 3 | 1 | 1 | 1 | 3 | 1 | 3 | 1 | 2 | 1 | 1 | 2 | 1 |
| 2 | 1 | 1 | 3 | 2 | 1 | 1 | 3 | 2 | 1 | 3 | 1 | 2 | 3 | 2 | 2 | 1 | 1 | 1 | 3 | 1 | 3 | 1 | 2 |
| 1 | 1 | 1 | 2 | 1 | 3 | 1 | 1 | 1 | 3 | 1 | 1 | 2 | 2 | 3 | 2 | 1 | 3 | 1 | 3 | 2 | 1 | 3 | 2 |
| 1 | 2 | 1 | 3 | 1 | 2 | 2 | 2 | 1 | 1 | 3 | 2 | 3 | 1 | 1 | 3 | 1 | 3 | 1 | 3 | 2 | 2 | 1 | 2 |
| 3 | 1 | 1 | 2 | 3 | 2 | 2 | 2 | 3 | 2 | 1 | 1 | 1 | 2 | 3 | 2 | 1 | 2 | 1 | 3 | 1 | 2 | 1 | 3 |
| 1 | 1 | 1 | 2 | 1 | 3 | 1 | 1 | 2 | 3 | 1 | 3 | 2 | 1 | 3 | 2 | 3 | 1 | 1 | 1 | 2 | 1 | 2 | 3 |
| 2 | 2 | 3 | 1 | 1 | 2 | 2 | 1 | 2 | 3 | 2 | 1 | 3 | 1 | 3 | 1 | 1 | 1 | 3 | 2 | 1 | 1 | 1 | 3 |
| 2 | 1 | 3 | 2 | 1 | 1 | 1 | 2 | 2 | 3 | 1 | 3 | 1 | 3 | 2 | 1 | 3 | 2 | 2 | 3 | 1 | 1 | 2 | 2 |
| 2 | 3 | 2 | 1 | 1 | 1 | 3 | 2 | 3 | 2 | 2 | 2 | 1 | 2 | 1 | 3 | 2 | 3 | 2 | 3 | 2 | 1 | 1 | 2 |
| 1 | 2 | 1 | 2 | 3 | 1 | 2 | 2 | 2 | 3 | 1 | 3 | 1 | 2 | 3 | 1 | 3 | 1 | 1 | 2 | 3 | 2 | 1 | 1 |
| 1 | 1 | 2 | 1 | 2 | 2 | 3 | 1 | 2 | 1 | 2 | 3 | 2 | 3 | 2 | 2 | 3 | 2 | 3 | 1 | 1 | 3 | 2 | 1 |
| 1 | 3 | 2 | 3 | 1 | 3 | 1 | 2 | 2 | 1 | 2 | 3 | 1 | 3 | 2 | 1 | 2 | 2 | 3 | 1 | 2 | 2 | 2 | 1 |
| 2 | 2 | 3 | 2 | 1 | 2 | 2 | 2 | 1 | 3 | 1 | 2 | 1 | 3 | 2 | 3 | 1 | 3 | 1 | 2 | 2 | 1 | 2 | 3 |
| 1 | 2 | 1 | 3 | 1 | 1 | 1 | 2 | 3 | 1 | 1 | 1 | 3 | 1 | 2 | 1 | 3 | 1 | 2 | 1 | 3 | 1 | 1 | 3 |
| 3 | 1 | 2 | 2 | 3 | 2 | 1 | 2 | 1 | 2 | 3 | 2 | 1 | 1 | 1 | 3 | 2 | 1 | 3 | 2 | 2 | 2 | 1 | 3 |
| 2 | 1 | 2 | 3 | 1 | 1 | 2 | 3 | 2 | 2 | 1 | 2 | 2 | 3 | 2 | 3 | 2 | 3 | 2 | 2 | 3 | 1 | 2 | 2 |
| 3 | 1 | 2 | 1 | 2 | 2 | 1 | 3 | 2 | 1 | 3 | 1 | 3 | 2 | 1 | 1 | 3 | 2 | 1 | 2 | 1 | 2 | 2 | 3 |
| 2 | 3 | 1 | 3 | 1 | 2 | 3 | 1 | 1 | 2 | 2 | 2 | 3 | 2 | 3 | 2 | 2 | 1 | 2 | 3 | 1 | 2 | 1 | 2 |
| 2 | 1 | 2 | 3 | 1 | 1 | 2 | 3 | 1 | 1 | 3 | 2 | 1 | 1 | 1 | 3 | 1 | 3 | 1 | 2 | 3 | 2 | 1 | 1 |
| 3 | 1 | 3 | 2 | 3 | 1 | 1 | 2 | 2 | 2 | 3 | 2 | 2 | 3 | 2 | 1 | 1 | 2 | 2 | 2 | 3 | 2 | 2 | 2 |
| 1 | 3 | 1 | 1 | 1 | 2 | 2 | 3 | 2 | 1 | 3 | 1 | 3 | 2 | 2 | 1 | 1 | 2 | 2 | 3 | 2 | 3 | 2 | 1 |
| 3 | 2 | 3 | 2 | 2 | 1 | 1 | 2 | 3 | 1 | 1 | 1 | 3 | 2 | 2 | 3 | 2 | 3 | 1 | 1 | 2 | 1 | 1 | 2 |
| 2 | 3 | 2 | 3 | 1 | 2 | 2 | 2 | 3 | 2 | 2 | 1 | 1 | 3 | 1 | 1 | 3 | 1 | 2 | 2 | 1 | 1 | 2 | 3 |
| 1 | 3 | 2 | 1 | 3 | 2 | 1 | 2 | 2 | 3 | 2 | 1 | 1 | 1 | 3 | 2 | 1 | 2 | 1 | 1 | 1 | 3 | 1 | 3 |
| 2 | 3 | 1 | 2 | 2 | 3 | 2 | 2 | 3 | 2 | 1 | 2 | 1 | 3 | 2 | 2 | 1 | 2 | 2 | 3 | 2 | 3 | 2 | 1 |
| 3 | 1 | 2 | 2 | 3 | 2 | 1 | 3 | 2 | 2 | 2 | 1 | 1 | 2 | 3 | 2 | 2 | 1 | 1 | 3 | 1 | 1 | 2 | 3 |
| 1 | 2 | 3 | 1 | 1 | 1 | 2 | 1 | 1 | 3 | 1 | 1 | 1 | 2 | 2 | 3 | 1 | 3 | 2 | 1 | 3 | 1 | 3 | 1 |
| 2 | 1 | 2 | 3 | 1 | 2 | 3 | 1 | 2 | 1 | 2 | 2 | 2 | 3 | 2 | 2 | 3 | 2 | 1 | 2 | 3 | 2 | 3 | 2 |
| 2 | 2 | 2 | 1 | 3 | 1 | 3 | 2 | 2 | 2 | 3 | 1 | 2 | 2 | 1 | 3 | 2 | 1 | 2 | 3 | 2 | 2 | 2 | 3 |
| 1 | 1 | 2 | 1 | 1 | 3 | 1 | 3 | 1 | 2 | 2 | 3 | 2 | 3 | 1 | 2 | 3 | 1 | 3 | 1 | 1 | 1 | 2 | 1 |
| 1 | 1 | 2 | 3 | 1 | 1 | 2 | 1 | 3 | 1 | 1 | 2 | 1 | 3 | 1 | 3 | 1 | 1 | 2 | 3 | 2 | 1 | 3 | 1 |
| 3 | 2 | 1 | 3 | 2 | 1 | 3 | 2 | 1 | 1 | 2 | 2 | 2 | 3 | 1 | 1 | 2 | 3 | 2 | 2 | 2 | 3 | 1 | 1 |
| 1 | 3 | 2 | 3 | 1 | 3 | 2 | 1 | 1 | 2 | 2 | 3 | 1 | 2 | 2 | 3 | 1 | 2 | 2 | 3 | 2 | 2 | 1 | 1 |
| 3 | 1 | 1 | 2 | 1 | 1 | 2 | 3 | 2 | 2 | 2 | 1 | 3 | 2 | 3 | 2 | 3 | 2 | 2 | 2 | 3 | 1 | 1 | 1 |
| 1 | 2 | 1 | 2 | 3 | 1 | 1 | 1 | 3 | 2 | 1 | 3 | 1 | 3 | 1 | 1 | 1 | 3 | 2 | 3 | 2 | 2 | 1 | 2 |
| 2 | 3 | 1 | 3 | 2 | 2 | 1 | 2 | 2 | 3 | 2 | 1 | 2 | 2 | 2 | 1 | 3 | 2 | 2 | 2 | 3 | 1 | 1 | 3 |
| 2 | 1 | 3 | 2 | 2 | 3 | 1 | 3 | 2 | 2 | 2 | 1 | 1 | 1 | 3 | 2 | 2 | 3 | 1 | 1 | 1 | 3 | 1 | 1 |
| 2 | 1 | 1 | 1 | 3 | 1 | 3 | 2 | 3 | 1 | 2 | 3 | 2 | 1 | 1 | 1 | 2 | 1 | 3 | 1 | 1 | 3 | 2 | 2 |
| 2 | 3 | 2 | 1 | 3 | 2 | 3 | 2 | 2 | 2 | 1 | 3 | 1 | 3 | 2 | 1 | 1 | 3 | 2 | 2 | 1 | 2 | 2 | 1 |
| 1 | 3 | 1 | 3 | 1 | 2 | 2 | 1 | 1 | 2 | 3 | 2 | 3 | 2 | 2 | 3 | 1 | 1 | 1 | 3 | 1 | 2 | 2 | 1 |
| 3 | 2 | 1 | 1 | 2 | 1 | 1 | 3 | 2 | 2 | 3 | 2 | 3 | 1 | 1 | 1 | 3 | 1 | 1 | 3 | 1 | 2 | 2 | 1 |
| 3 | 1 | 3 | 1 | 2 | 3 | 2 | 2 | 1 | 2 | 1 | 3 | 1 | 2 | 1 | 1 | 2 | 3 | 1 | 1 | 1 | 3 | 1 | 1 |
| 2 | 2 | 2 | 1 | 3 | 2 | 2 | 3 | 1 | 2 | 2 | 3 | 2 | 2 | 3 | 1 | 1 | 2 | 1 | 3 | 1 | 3 | 2 | 1 |
| 1 | 2 | 2 | 1 | 2 | 2 | 3 | 1 | 1 | 1 | 3 | 2 | 1 | 3 | 1 | 2 | 3 | 2 | 2 | 1 | 3 | 1 | 2 | 3 |
| 2 | 2 | 2 | 1 | 2 | 3 | 2 | 3 | 2 | 3 | 1 | 2 | 2 | 3 | 1 | 3 | 2 | 3 | 2 | 2 | 2 | 1 | 1 | 2 |
| 2 | 1 | 2 | 2 | 2 | 1 | 3 | 2 | 2 | 1 | 3 | 1 | 2 | 1 | 3 | 1 | 2 | 1 | 3 | 1 | 3 | 1 | 3 | 2 |
| 1 | 2 | 3 | 2 | 3 | 2 | 2 | 2 | 1 | 2 | 3 | 2 | 3 | 1 | 1 | 1 | 3 | 1 | 2 | 2 | 2 | 3 | 2 | 1 |
| 1 | 2 | 1 | 3 | 2 | 1 | 1 | 2 | 2 | 1 | 3 | 1 | 1 | 3 | 1 | 3 | 1 | 1 | 3 | 1 | 1 | 2 | 3 | 2 |
| 2 | 1 | 2 | 3 | 1 | 3 | 2 | 3 | 1 | 2 | 2 | 1 | 3 | 1 | 1 | 2 | 2 | 3 | 2 | 1 | 2 | 2 | 2 | 3 |
| 2 | 2 | 1 | 1 | 2 | 3 | 2 | 1 | 2 | 2 | 3 | 2 | 2 | 2 | 1 | 1 | 1 | 3 | 1 | 3 | 2 | 3 | 2 | 3 |
| 1 | 2 | 1 | 3 | 1 | 3 | 1 | 1 | 2 | 2 | 1 | 1 | 3 | 1 | 1 | 2 | 2 | 3 | 2 | 2 | 2 | 3 | 1 | 3 |
| 3 | 2 | 2 | 1 | 2 | 1 | 1 | 3 | 2 | 1 | 3 | 1 | 1 | 1 | 2 | 3 | 2 | 1 | 2 | 1 | 3 | 1 | 1 | 3 |
| 1 | 3 | 2 | 1 | 1 | 2 | 2 | 1 | 3 | 2 | 2 | 2 | 3 | 1 | 1 | 1 | 2 | 3 | 2 | 3 | 2 | 1 | 3 | 2 |
| 3 | 1 | 1 | 1 | 3 | 1 | 2 | 2 | 1 | 2 | 3 | 1 | 2 | 2 | 3 | 2 | 1 | 1 | 1 | 3 | 2 | 3 | 1 | 2 |
| 3 | 2 | 1 | 1 | 3 | 1 | 2 | 2 | 1 | 3 | 1 | 1 | 3 | 2 | 2 | 1 | 1 | 2 | 3 | 1 | 1 | 3 | 1 | 1 |
| 3 | 1 | 3 | 1 | 1 | 2 | 3 | 2 | 2 | 3 | 1 | 1 | 2 | 1 | 1 | 3 | 1 | 1 | 3 | 2 | 1 | 1 | 2 | 2 |
| 2 | 2 | 1 | 1 | 3 | 1 | 3 | 2 | 3 | 2 | 2 | 2 | 3 | 1 | 1 | 2 | 1 | 3 | 2 | 3 | 2 | 2 | 2 | 1 |
| 1 | 2 | 1 | 1 | 1 | 3 | 1 | 1 | 1 | 3 | 1 | 3 | 2 | 1 | 2 | 3 | 1 | 3 | 1 | 2 | 2 | 1 | 2 | 3 |
| 1 | 3 | 2 | 2 | 1 | 2 | 2 | 3 | 1 | 2 | 2 | 3 | 1 | 1 | 3 | 1 | 2 | 3 | 1 | 3 | 1 | 1 | 1 | 2 |
| 3 | 2 | 2 | 2 | 3 | 2 | 3 | 2 | 2 | 2 | 3 | 2 | 1 | 2 | 1 | 1 | 3 | 2 | 2 | 3 | 2 | 2 | 1 | 1 |
| 2 | 2 | 3 | 2 | 1 | 2 | 3 | 2 | 3 | 1 | 3 | 2 | 2 | 2 | 1 | 3 | 1 | 2 | 2 | 1 | 1 | 2 | 3 | 1 |
| 2 | 1 | 3 | 2 | 2 | 1 | 1 | 1 | 3 | 2 | 1 | 2 | 1 | 3 | 2 | 2 | 3 | 2 | 2 | 2 | 3 | 1 | 3 | 2 |
| 1 | 1 | 1 | 2 | 2 | 2 | 3 | 2 | 3 | 2 | 2 | 3 | 1 | 3 | 1 | 2 | 2 | 2 | 3 | 2 | 1 | 2 | 1 | 3 |
| 2 | 1 | 2 | 2 | 1 | 3 | 2 | 3 | 2 | 2 | 1 | 2 | 3 | 1 | 2 | 1 | 1 | 1 | 3 | 1 | 3 | 1 | 1 | 3 |
| 2 | 1 | 2 | 1 | 1 | 3 | 1 | 1 | 3 | 2 | 1 | 1 | 2 | 2 | 2 | 3 | 1 | 3 | 1 | 1 | 3 | 1 | 3 | 2 |
| 2 | 1 | 1 | 3 | 2 | 2 | 3 | 1 | 3 | 1 | 2 | 3 | 2 | 2 | 2 | 3 | 2 | 2 | 2 | 3 | 1 | 2 | 1 | 1 |
| 3 | 2 | 3 | 2 | 1 | 3 | 1 | 2 | 2 | 2 | 1 | 2 | 3 | 1 | 1 | 2 | 2 | 3 | 1 | 3 | 2 | 1 | 1 | 2 |
| 2 | 1 | 2 | 1 | 3 | 1 | 3 | 1 | 1 | 3 | 2 | 3 | 2 | 2 | 2 | 1 | 3 | 2 | 2 | 3 | 2 | 1 | 2 | 1 |
| 1 | 2 | 1 | 1 | 1 | 3 | 1 | 1 | 3 | 1 | 1 | 2 | 1 | 3 | 2 | 2 | 3 | 2 | 2 | 3 | 2 | 3 | 2 | 1 |
| 1 | 3 | 1 | 2 | 2 | 3 | 1 | 1 | 1 | 2 | 1 | 3 | 1 | 2 | 2 | 1 | 3 | 1 | 1 | 1 | 3 | 2 | 2 | 3 |
| 3 | 2 | 2 | 3 | 2 | 2 | 1 | 2 | 1 | 1 | 3 | 1 | 1 | 1 | 2 | 1 | 3 | 2 | 2 | 2 | 3 | 2 | 2 | 3 |
| 1 | 3 | 1 | 1 | 1 | 2 | 1 | 3 | 1 | 3 | 2 | 1 | 1 | 3 | 1 | 3 | 2 | 3 | 2 | 2 | 2 | 1 | 1 | 1 |
| 1 | 3 | 1 | 3 | 1 | 2 | 1 | 3 | 2 | 1 | 3 | 2 | 1 | 1 | 1 | 2 | 1 | 3 | 2 | 2 | 1 | 2 | 2 | 3 |
| 1 | 1 | 1 | 2 | 3 | 1 | 2 | 2 | 3 | 2 | 3 | 2 | 1 | 1 | 3 | 2 | 2 | 1 | 2 | 3 | 2 | 1 | 2 | 3 |
| 1 | 1 | 3 | 1 | 1 | 3 | 2 | 1 | 1 | 3 | 1 | 3 | 1 | 3 | 1 | 1 | 1 | 2 | 2 | 2 | 3 | 1 | 1 | 2 |
| 3 | 2 | 3 | 2 | 3 | 2 | 1 | 2 | 2 | 2 | 1 | 3 | 2 | 2 | 3 | 1 | 2 | 1 | 1 | 2 | 2 | 3 | 1 | 2 |
| 1 | 2 | 2 | 3 | 2 | 2 | 3 | 2 | 2 | 3 | 2 | 2 | 3 | 1 | 3 | 1 | 1 | 1 | 2 | 3 | 2 | 1 | 2 | 2 |
| 1 | 3 | 1 | 2 | 1 | 1 | 3 | 2 | 2 | 1 | 1 | 1 | 3 | 2 | 1 | 1 | 1 | 3 | 1 | 3 | 1 | 1 | 2 | 3 |
| 2 | 1 | 3 | 2 | 2 | 3 | 1 | 1 | 3 | 2 | 2 | 1 | 3 | 2 | 2 | 2 | 1 | 1 | 3 | 2 | 3 | 2 | 2 | 1 |
| 1 | 3 | 2 | 1 | 1 | 3 | 1 | 1 | 2 | 3 | 2 | 1 | 1 | 2 | 1 | 2 | 3 | 1 | 2 | 3 | 1 | 2 | 1 | 3 |
| 1 | 2 | 3 | 1 | 3 | 1 | 2 | 2 | 3 | 1 | 1 | 1 | 3 | 1 | 2 | 2 | 2 | 1 | 2 | 3 | 1 | 1 | 2 | 3 |
| 2 | 3 | 1 | 2 | 2 | 3 | 1 | 1 | 2 | 2 | 1 | 3 | 1 | 3 | 1 | 3 | 1 | 1 | 2 | 3 | 2 | 1 | 2 | 1 |
| 1 | 3 | 2 | 2 | 1 | 3 | 2 | 1 | 1 | 3 | 1 | 3 | 1 | 1 | 2 | 1 | 2 | 1 | 3 | 2 | 3 | 1 | 1 | 2 |
| 1 | 2 | 2 | 1 | 1 | 3 | 1 | 2 | 2 | 3 | 2 | 1 | 2 | 1 | 3 | 2 | 2 | 1 | 3 | 2 | 3 | 1 | 2 | 3 |
| 3 | 1 | 3 | 1 | 2 | 1 | 1 | 1 | 3 | 1 | 1 | 2 | 2 | 3 | 1 | 1 | 1 | 2 | 1 | 3 | 1 | 1 | 3 | 1 |
| 1 | 3 | 1 | 3 | 2 | 1 | 1 | 1 | 2 | 3 | 2 | 2 | 1 | 1 | 3 | 1 | 1 | 1 | 3 | 1 | 1 | 3 | 2 | 2 |
| 1 | 1 | 1 | 3 | 2 | 2 | 2 | 3 | 2 | 2 | 1 | 2 | 3 | 2 | 3 | 2 | 3 | 1 | 1 | 3 | 1 | 1 | 2 | 2 |
| 1 | 2 | 2 | 3 | 2 | 3 | 2 | 2 | 2 | 1 | 1 | 3 | 1 | 1 | 1 | 2 | 1 | 2 | 3 | 1 | 2 | 3 | 1 | 3 |
| 2 | 1 | 2 | 2 | 3 | 1 | 1 | 1 | 2 | 3 | 1 | 3 | 1 | 2 | 3 | 2 | 1 | 2 | 3 | 2 | 1 | 3 | 2 | 2 |
| 1 | 2 | 2 | 2 | 3 | 2 | 3 | 2 | 3 | 1 | 2 | 3 | 2 | 2 | 2 | 3 | 1 | 1 | 1 | 2 | 1 | 2 | 3 | 1 |
| 2 | 1 | 1 | 3 | 1 | 2 | 1 | 1 | 2 | 1 | 3 | 2 | 3 | 1 | 3 | 1 | 3 | 1 | 1 | 1 | 2 | 2 | 3 | 1 |
| 1 | 2 | 2 | 2 | 1 | 2 | 3 | 1 | 2 | 2 | 1 | 3 | 2 | 3 | 2 | 1 | 1 | 3 | 2 | 3 | 2 | 2 | 3 | 2 |
| 3 | 1 | 2 | 2 | 1 | 1 | 3 | 1 | 1 | 2 | 1 | 1 | 1 | 3 | 2 | 3 | 2 | 3 | 1 | 1 | 3 | 1 | 1 | 2 |
| 3 | 2 | 1 | 1 | 2 | 2 | 3 | 1 | 2 | 3 | 1 | 1 | 3 | 1 | 3 | 2 | 2 | 1 | 3 | 2 | 2 | 2 | 1 | 2 |
| 2 | 3 | 2 | 3 | 2 | 2 | 1 | 2 | 3 | 2 | 2 | 1 | 2 | 1 | 1 | 3 | 1 | 1 | 3 | 2 | 3 | 1 | 2 | 1 |
| 1 | 3 | 1 | 3 | 1 | 1 | 1 | 2 | 2 | 3 | 1 | 1 | 2 | 2 | 2 | 1 | 3 | 1 | 1 | 1 | 2 | 3 | 2 | 3 |
| 2 | 2 | 1 | 2 | 2 | 3 | 1 | 1 | 2 | 3 | 2 | 3 | 1 | 3 | 1 | 1 | 1 | 3 | 2 | 1 | 2 | 2 | 2 | 3 |
| 2 | 3 | 2 | 2 | 1 | 1 | 2 | 3 | 1 | 3 | 1 | 1 | 3 | 1 | 2 | 1 | 1 | 2 | 3 | 1 | 2 | 1 | 3 | 2 |
| 3 | 1 | 1 | 1 | 3 | 2 | 1 | 2 | 2 | 2 | 3 | 2 | 2 | 3 | 1 | 2 | 2 | 1 | 2 | 2 | 3 | 2 | 2 | 3 |
| 2 | 1 | 3 | 2 | 2 | 2 | 1 | 2 | 3 | 2 | 1 | 3 | 2 | 2 | 1 | 1 | 2 | 2 | 3 | 2 | 2 | 3 | 1 | 3 |
| 3 | 2 | 2 | 3 | 1 | 1 | 1 | 3 | 1 | 2 | 1 | 3 | 2 | 2 | 2 | 3 | 1 | 2 | 1 | 2 | 3 | 2 | 1 | 2 |
| 2 | 2 | 1 | 3 | 1 | 1 | 3 | 1 | 2 | 1 | 3 | 1 | 2 | 2 | 1 | 2 | 2 | 3 | 1 | 3 | 1 | 1 | 1 | 3 |
| 1 | 1 | 2 | 1 | 1 | 2 | 3 | 2 | 2 | 3 | 2 | 3 | 1 | 1 | 1 | 2 | 1 | 3 | 1 | 2 | 3 | 2 | 3 | 1 |
| 1 | 3 | 2 | 1 | 1 | 3 | 1 | 1 | 1 | 3 | 2 | 2 | 2 | 1 | 3 | 2 | 2 | 2 | 1 | 3 | 2 | 2 | 1 | 3 |
| 2 | 1 | 3 | 2 | 2 | 2 | 1 | 1 | 2 | 3 | 1 | 3 | 1 | 2 | 3 | 2 | 2 | 2 | 3 | 1 | 2 | 1 | 2 | 3 |
| 2 | 2 | 1 | 1 | 1 | 3 | 1 | 2 | 3 | 2 | 2 | 1 | 1 | 1 | 3 | 1 | 1 | 2 | 3 | 1 | 3 | 2 | 3 | 1 |
| 1 | 1 | 1 | 3 | 2 | 3 | 2 | 3 | 2 | 1 | 2 | 1 | 2 | 3 | 2 | 2 | 1 | 3 | 1 | 1 | 1 | 3 | 2 | 1 |
| 1 | 2 | 2 | 1 | 1 | 3 | 2 | 2 | 1 | 2 | 3 | 2 | 3 | 2 | 2 | 2 | 1 | 2 | 3 | 2 | 3 | 2 | 2 | 3 |
| 2 | 2 | 2 | 3 | 1 | 1 | 3 | 1 | 1 | 3 | 2 | 3 | 2 | 2 | 2 | 3 | 2 | 1 | 2 | 2 | 1 | 2 | 3 | 2 |
| 2 | 3 | 2 | 2 | 1 | 1 | 3 | 1 | 1 | 3 | 2 | 2 | 2 | 1 | 3 | 2 | 2 | 1 | 1 | 1 | 3 | 2 | 2 | 3 |
| 2 | 2 | 2 | 1 | 1 | 3 | 2 | 1 | 2 | 1 | 1 | 3 | 1 | 2 | 2 | 3 | 2 | 3 | 2 | 3 | 1 | 3 | 1 | 2 |
| 1 | 3 | 1 | 2 | 1 | 2 | 2 | 2 | 3 | 1 | 2 | 1 | 3 | 1 | 2 | 1 | 3 | 1 | 1 | 3 | 1 | 1 | 1 | 3 |
| 1 | 2 | 2 | 2 | 1 | 3 | 1 | 3 | 2 | 2 | 3 | 2 | 1 | 1 | 3 | 1 | 1 | 3 | 1 | 2 | 1 | 2 | 2 | 3 |
| 3 | 1 | 3 | 1 | 1 | 1 | 2 | 2 | 3 | 2 | 1 | 1 | 2 | 2 | 3 | 2 | 2 | 1 | 3 | 1 | 3 | 2 | 1 | 2 |
| 3 | 1 | 1 | 3 | 2 | 1 | 2 | 1 | 2 | 3 | 2 | 2 | 1 | 1 | 3 | 1 | 2 | 3 | 2 | 1 | 1 | 2 | 1 | 3 |
| 1 | 1 | 2 | 1 | 2 | 2 | 3 | 1 | 1 | 3 | 1 | 2 | 3 | 2 | 1 | 3 | 2 | 3 | 1 | 3 | 2 | 2 | 1 | 2 |
| 3 | 1 | 3 | 2 | 2 | 2 | 1 | 3 | 1 | 1 | 1 | 2 | 3 | 1 | 2 | 1 | 1 | 1 | 3 | 1 | 1 | 2 | 2 | 3 |
| 2 | 1 | 1 | 3 | 1 | 1 | 1 | 2 | 3 | 1 | 3 | 2 | 2 | 1 | 2 | 1 | 2 | 3 | 2 | 2 | 3 | 1 | 3 | 1 |
| 2 | 2 | 3 | 1 | 2 | 1 | 2 | 1 | 1 | 3 | 1 | 1 | 3 | 2 | 2 | 3 | 2 | 3 | 1 | 2 | 1 | 1 | 3 | 2 |
| 1 | 1 | 3 | 2 | 3 | 2 | 2 | 2 | 1 | 1 | 2 | 3 | 2 | 1 | 1 | 3 | 1 | 3 | 1 | 1 | 2 | 3 | 1 | 1 |
| 3 | 2 | 2 | 3 | 2 | 3 | 1 | 3 | 1 | 1 | 2 | 2 | 1 | 3 | 1 | 1 | 1 | 2 | 1 | 3 | 2 | 1 | 2 | 1 |
| 2 | 2 | 2 | 1 | 3 | 2 | 2 | 2 | 3 | 1 | 2 | 3 | 2 | 3 | 2 | 2 | 2 | 1 | 2 | 3 | 1 | 3 | 1 | 2 |
| 3 | 2 | 1 | 1 | 2 | 2 | 3 | 1 | 1 | 1 | 3 | 2 | 1 | 2 | 3 | 1 | 3 | 2 | 1 | 3 | 2 | 1 | 1 | 2 |
| 2 | 1 | 3 | 2 | 2 | 3 | 1 | 1 | 2 | 1 | 1 | 3 | 1 | 2 | 2 | 3 | 1 | 3 | 1 | 3 | 1 | 1 | 1 | 2 |
| 2 | 2 | 1 | 1 | 3 | 2 | 3 | 1 | 1 | 3 | 2 | 3 | 2 | 2 | 3 | 2 | 2 | 2 | 1 | 2 | 2 | 3 | 1 | 1 |
| 1 | 2 | 2 | 3 | 1 | 2 | 2 | 2 | 3 | 2 | 2 | 3 | 1 | 1 | 1 | 2 | 1 | 1 | 3 | 2 | 3 | 2 | 2 | 3 |
| 2 | 3 | 1 | 1 | 2 | 2 | 3 | 2 | 2 | 3 | 1 | 2 | 1 | 1 | 3 | 2 | 2 | 1 | 2 | 3 | 1 | 1 | 3 | 1 |
| 3 | 2 | 2 | 2 | 3 | 2 | 2 | 1 | 2 | 2 | 3 | 1 | 3 | 2 | 1 | 1 | 3 | 2 | 2 | 3 | 1 | 1 | 2 | 2 |
| 2 | 3 | 1 | 2 | 2 | 2 | 1 | 3 | 2 | 1 | 2 | 3 | 2 | 1 | 2 | 2 | 1 | 3 | 1 | 3 | 2 | 2 | 3 | 1 |
| 2 | 1 | 1 | 1 | 2 | 1 | 3 | 1 | 3 | 1 | 2 | 3 | 1 | 3 | 1 | 1 | 2 | 1 | 1 | 3 | 1 | 1 | 1 | 3 |
| 1 | 3 | 1 | 1 | 2 | 3 | 2 | 2 | 1 | 2 | 1 | 2 | 3 | 2 | 1 | 3 | 1 | 3 | 1 | 1 | 1 | 2 | 2 | 3 |
| 1 | 2 | 2 | 2 | 1 | 2 | 3 | 2 | 1 | 3 | 2 | 2 | 3 | 1 | 3 | 1 | 3 | 2 | 3 | 1 | 2 | 1 | 1 | 1 |
| 3 | 2 | 1 | 1 | 1 | 3 | 1 | 2 | 1 | 3 | 2 | 2 | 2 | 3 | 1 | 3 | 2 | 1 | 1 | 2 | 2 | 2 | 3 | 1 |
| 3 | 1 | 1 | 1 | 2 | 1 | 3 | 2 | 1 | 2 | 1 | 1 | 2 | 3 | 2 | 2 | 1 | 1 | 3 | 2 | 3 | 1 | 3 | 1 |
| 1 | 2 | 2 | 3 | 2 | 1 | 2 | 1 | 2 | 2 | 3 | 2 | 3 | 2 | 2 | 3 | 1 | 1 | 3 | 1 | 1 | 1 | 3 | 2 |
| 3 | 1 | 3 | 2 | 2 | 1 | 1 | 3 | 2 | 3 | 2 | 1 | 1 | 1 | 2 | 3 | 1 | 1 | 1 | 2 | 3 | 2 | 1 | 1 |
| 1 | 2 | 1 | 3 | 1 | 2 | 2 | 3 | 2 | 3 | 2 | 3 | 1 | 1 | 1 | 3 | 1 | 1 | 1 | 3 | 1 | 1 | 2 | 2 |
| 2 | 2 | 1 | 1 | 2 | 1 | 3 | 1 | 1 | 3 | 2 | 2 | 2 | 3 | 2 | 1 | 3 | 2 | 1 | 2 | 3 | 1 | 2 | 3 |
| 2 | 2 | 3 | 2 | 1 | 2 | 3 | 2 | 3 | 1 | 3 | 1 | 1 | 2 | 1 | 1 | 1 | 3 | 2 | 2 | 2 | 1 | 3 | 2 |
| 3 | 2 | 3 | 1 | 2 | 2 | 1 | 3 | 1 | 2 | 1 | 2 | 3 | 1 | 2 | 3 | 1 | 2 | 1 | 2 | 3 | 1 | 1 | 2 |
| 2 | 3 | 1 | 1 | 3 | 1 | 1 | 3 | 1 | 1 | 2 | 2 | 2 | 1 | 3 | 1 | 2 | 2 | 2 | 3 | 2 | 1 | 1 | 3 |
| 2 | 3 | 2 | 1 | 2 | 3 | 1 | 2 | 2 | 1 | 2 | 2 | 3 | 1 | 2 | 2 | 1 | 3 | 2 | 3 | 2 | 3 | 2 | 2 |
| 2 | 3 | 2 | 3 | 1 | 1 | 1 | 3 | 1 | 3 | 1 | 1 | 2 | 3 | 1 | 2 | 1 | 3 | 1 | 2 | 1 | 2 | 2 | 2 |
| 1 | 1 | 2 | 2 | 3 | 1 | 1 | 1 | 2 | 3 | 1 | 3 | 2 | 3 | 2 | 3 | 2 | 2 | 2 | 1 | 1 | 3 | 1 | 1 |
| 1 | 2 | 2 | 1 | 2 | 1 | 3 | 1 | 3 | 2 | 2 | 1 | 3 | 2 | 2 | 2 | 1 | 3 | 1 | 1 | 2 | 3 | 1 | 3 |
| 1 | 1 | 1 | 3 | 1 | 2 | 1 | 3 | 1 | 1 | 1 | 2 | 2 | 3 | 1 | 3 | 2 | 3 | 2 | 1 | 2 | 3 | 1 | 2 |
| 3 | 2 | 1 | 3 | 2 | 2 | 2 | 3 | 2 | 2 | 1 | 1 | 2 | 3 | 2 | 2 | 3 | 2 | 1 | 2 | 1 | 1 | 2 | 3 |
| 1 | 3 | 1 | 3 | 1 | 2 | 1 | 2 | 2 | 1 | 3 | 1 | 1 | 2 | 3 | 2 | 1 | 1 | 3 | 1 | 1 | 2 | 1 | 3 |
| 1 | 3 | 1 | 1 | 3 | 2 | 2 | 2 | 3 | 1 | 1 | 1 | 2 | 1 | 2 | 3 | 1 | 2 | 1 | 3 | 1 | 1 | 2 | 3 |
| 2 | 1 | 3 | 1 | 1 | 2 | 3 | 2 | 1 | 1 | 1 | 3 | 2 | 2 | 2 | 1 | 3 | 2 | 1 | 2 | 1 | 3 | 1 | 3 |
| 1 | 3 | 2 | 1 | 3 | 1 | 2 | 3 | 2 | 1 | 2 | 3 | 2 | 2 | 1 | 1 | 2 | 3 | 2 | 3 | 1 | 1 | 2 | 1 |
| 2 | 3 | 1 | 1 | 1 | 3 | 2 | 3 | 1 | 1 | 1 | 2 | 1 | 2 | 3 | 1 | 1 | 1 | 2 | 3 | 2 | 2 | 3 | 2 |
| 1 | 2 | 1 | 3 | 2 | 1 | 2 | 1 | 2 | 2 | 3 | 1 | 3 | 2 | 2 | 2 | 3 | 2 | 1 | 2 | 3 | 1 | 1 | 3 |
| 3 | 1 | 1 | 3 | 1 | 1 | 1 | 2 | 3 | 2 | 2 | 2 | 3 | 2 | 1 | 3 | 1 | 1 | 2 | 1 | 1 | 3 | 2 | 1 |
| 1 | 1 | 2 | 3 | 1 | 3 | 2 | 1 | 2 | 2 | 3 | 1 | 1 | 3 | 1 | 1 | 1 | 2 | 3 | 2 | 1 | 2 | 1 | 3 |
| 3 | 2 | 3 | 1 | 2 | 1 | 3 | 1 | 1 | 2 | 2 | 2 | 3 | 2 | 3 | 2 | 2 | 2 | 1 | 1 | 2 | 3 | 1 | 1 |
| 2 | 3 | 2 | 1 | 3 | 2 | 1 | 2 | 3 | 1 | 1 | 3 | 1 | 1 | 2 | 1 | 1 | 2 | 3 | 1 | 1 | 1 | 2 | 3 |
| 1 | 2 | 1 | 3 | 1 | 1 | 3 | 2 | 2 | 1 | 1 | 2 | 3 | 1 | 2 | 1 | 1 | 2 | 2 | 3 | 2 | 3 | 2 | 3 |
| 3 | 2 | 3 | 1 | 2 | 2 | 3 | 2 | 1 | 1 | 3 | 2 | 1 | 1 | 3 | 2 | 1 | 1 | 1 | 3 | 1 | 2 | 1 | 1 |
| 2 | 1 | 2 | 3 | 2 | 1 | 3 | 2 | 2 | 2 | 3 | 2 | 3 | 2 | 2 | 1 | 2 | 2 | 2 | 3 | 1 | 1 | 3 | 1 |
| 2 | 3 | 1 | 3 | 2 | 1 | 1 | 3 | 2 | 2 | 2 | 3 | 2 | 1 | 2 | 3 | 2 | 2 | 2 | 1 | 1 | 3 | 2 | 1 |
| 2 | 1 | 1 | 1 | 2 | 3 | 2 | 1 | 2 | 3 | 1 | 3 | 2 | 3 | 2 | 3 | 2 | 1 | 1 | 1 | 3 | 1 | 1 | 1 |
| 3 | 2 | 1 | 1 | 3 | 1 | 3 | 2 | 1 | 2 | 2 | 3 | 1 | 1 | 1 | 2 | 2 | 1 | 3 | 2 | 1 | 1 | 3 | 1 |
| 3 | 2 | 2 | 3 | 1 | 3 | 2 | 3 | 2 | 1 | 1 | 1 | 3 | 1 | 2 | 2 | 1 | 2 | 2 | 3 | 1 | 2 | 1 | 1 |
| 1 | 3 | 2 | 1 | 2 | 3 | 1 | 3 | 2 | 2 | 1 | 2 | 2 | 1 | 3 | 1 | 2 | 1 | 1 | 1 | 3 | 2 | 3 | 1 |
| 1 | 2 | 2 | 2 | 3 | 2 | 2 | 1 | 2 | 1 | 3 | 1 | 3 | 2 | 2 | 3 | 2 | 3 | 2 | 2 | 3 | 2 | 1 | 2 |
| 2 | 1 | 1 | 2 | 2 | 1 | 3 | 2 | 1 | 3 | 2 | 3 | 2 | 3 | 2 | 2 | 3 | 1 | 1 | 1 | 2 | 2 | 2 | 3 |
| 2 | 3 | 2 | 1 | 2 | 2 | 3 | 1 | 3 | 1 | 2 | 2 | 3 | 2 | 2 | 1 | 2 | 2 | 3 | 2 | 1 | 2 | 2 | 3 |
| 3 | 2 | 2 | 1 | 2 | 2 | 1 | 3 | 1 | 1 | 3 | 1 | 3 | 1 | 2 | 1 | 1 | 2 | 2 | 3 | 1 | 3 | 2 | 2 |
| 2 | 2 | 3 | 1 | 3 | 2 | 2 | 3 | 2 | 3 | 1 | 2 | 2 | 1 | 1 | 3 | 2 | 1 | 3 | 2 | 1 | 2 | 1 | 2 |
| 3 | 1 | 2 | 1 | 3 | 2 | 1 | 2 | 1 | 1 | 2 | 3 | 1 | 2 | 2 | 3 | 1 | 1 | 3 | 2 | 1 | 1 | 2 | 3 |
| 3 | 2 | 3 | 1 | 1 | 1 | 3 | 1 | 2 | 1 | 2 | 2 | 2 | 3 | 1 | 3 | 1 | 3 | 1 | 2 | 1 | 1 | 1 | 2 |
| 1 | 3 | 2 | 2 | 1 | 2 | 3 | 1 | 2 | 2 | 2 | 3 | 1 | 1 | 3 | 1 | 1 | 1 | 2 | 2 | 3 | 2 | 2 | 3 |
| 3 | 2 | 1 | 1 | 3 | 2 | 1 | 2 | 2 | 2 | 3 | 1 | 1 | 2 | 2 | 2 | 3 | 1 | 2 | 3 | 1 | 3 | 2 | 2 |
| 2 | 1 | 1 | 2 | 1 | 3 | 2 | 3 | 2 | 2 | 1 | 2 | 1 | 1 | 3 | 2 | 3 | 1 | 1 | 1 | 3 | 1 | 3 | 2 |
| 1 | 1 | 1 | 2 | 3 | 1 | 1 | 2 | 2 | 3 | 1 | 2 | 3 | 2 | 3 | 2 | 1 | 2 | 1 | 2 | 3 | 1 | 1 | 3 |
| 1 | 3 | 1 | 1 | 1 | 3 | 2 | 3 | 1 | 3 | 2 | 2 | 3 | 2 | 2 | 1 | 1 | 3 | 2 | 1 | 2 | 2 | 2 | 1 |
| 2 | 2 | 2 | 1 | 2 | 3 | 2 | 3 | 2 | 3 | 1 | 1 | 2 | 2 | 3 | 2 | 3 | 2 | 1 | 2 | 1 | 2 | 1 | 3 |
| 3 | 2 | 1 | 1 | 2 | 1 | 2 | 3 | 1 | 2 | 1 | 3 | 1 | 1 | 1 | 2 | 3 | 2 | 1 | 1 | 1 | 3 | 1 | 3 |
| 3 | 1 | 3 | 1 | 1 | 2 | 2 | 3 | 2 | 2 | 2 | 1 | 1 | 1 | 3 | 1 | 2 | 1 | 3 | 2 | 2 | 3 | 2 | 1 |
| 3 | 1 | 1 | 2 | 2 | 2 | 3 | 2 | 2 | 1 | 1 | 3 | 1 | 1 | 2 | 3 | 1 | 3 | 2 | 2 | 2 | 3 | 1 | 2 |
| 1 | 2 | 1 | 3 | 2 | 3 | 1 | 2 | 3 | 1 | 2 | 2 | 1 | 1 | 1 | 3 | 1 | 3 | 1 | 1 | 2 | 2 | 2 | 3 |
| 1 | 2 | 1 | 3 | 1 | 2 | 3 | 2 | 2 | 2 | 1 | 3 | 2 | 2 | 3 | 1 | 3 | 1 | 2 | 2 | 1 | 2 | 2 | 3 |
| 1 | 1 | 3 | 1 | 3 | 2 | 3 | 2 | 1 | 1 | 1 | 2 | 1 | 3 | 1 | 1 | 1 | 3 | 2 | 3 | 1 | 2 | 1 | 2 |
| 2 | 3 | 2 | 3 | 2 | 1 | 2 | 2 | 3 | 1 | 2 | 2 | 3 | 2 | 2 | 3 | 1 | 3 | 1 | 2 | 1 | 1 | 1 | 2 |
| 2 | 1 | 3 | 2 | 1 | 2 | 1 | 3 | 2 | 3 | 1 | 3 | 1 | 1 | 1 | 3 | 1 | 3 | 2 | 2 | 1 | 1 | 1 | 2 |
| 1 | 1 | 1 | 3 | 1 | 2 | 1 | 1 | 3 | 1 | 1 | 1 | 3 | 1 | 3 | 1 | 2 | 3 | 1 | 2 | 3 | 2 | 2 | 2 |
| 3 | 1 | 1 | 3 | 2 | 2 | 1 | 2 | 2 | 3 | 1 | 1 | 1 | 2 | 1 | 3 | 1 | 3 | 1 | 1 | 3 | 2 | 1 | 2 |
| 1 | 2 | 3 | 2 | 1 | 2 | 3 | 2 | 1 | 2 | 1 | 3 | 1 | 1 | 1 | 3 | 1 | 3 | 2 | 1 | 1 | 1 | 2 | 3 |
| 3 | 1 | 2 | 3 | 2 | 2 | 2 | 3 | 2 | 1 | 1 | 1 | 3 | 1 | 2 | 2 | 3 | 1 | 1 | 1 | 2 | 2 | 3 | 1 |
| 1 | 1 | 2 | 2 | 2 | 1 | 3 | 1 | 3 | 1 | 3 | 2 | 1 | 2 | 2 | 2 | 3 | 2 | 3 | 2 | 2 | 3 | 2 | 1 |
| 1 | 1 | 2 | 2 | 2 | 3 | 2 | 2 | 2 | 3 | 1 | 1 | 1 | 3 | 1 | 1 | 1 | 3 | 2 | 1 | 1 | 3 | 2 | 3 |
| 1 | 1 | 1 | 3 | 1 | 3 | 2 | 1 | 3 | 2 | 3 | 2 | 2 | 1 | 2 | 2 | 3 | 2 | 2 | 1 | 3 | 1 | 2 | 1 |
| 3 | 2 | 1 | 2 | 3 | 2 | 2 | 3 | 2 | 1 | 2 | 1 | 2 | 3 | 2 | 2 | 3 | 2 | 2 | 3 | 1 | 2 | 1 | 2 |
| 3 | 2 | 1 | 3 | 1 | 1 | 2 | 2 | 2 | 3 | 2 | 2 | 3 | 1 | 3 | 2 | 1 | 2 | 2 | 2 | 3 | 2 | 1 | 1 |
| 1 | 2 | 3 | 1 | 1 | 2 | 2 | 2 | 1 | 3 | 2 | 2 | 1 | 3 | 2 | 3 | 2 | 1 | 1 | 3 | 1 | 1 | 1 | 3 |
| 1 | 2 | 3 | 1 | 2 | 1 | 1 | 3 | 1 | 1 | 1 | 2 | 3 | 2 | 2 | 3 | 1 | 2 | 3 | 1 | 1 | 3 | 2 | 1 |
| 2 | 2 | 3 | 1 | 2 | 3 | 1 | 2 | 3 | 1 | 1 | 3 | 1 | 2 | 1 | 1 | 2 | 3 | 2 | 1 | 3 | 1 | 2 | 1 |
| 1 | 3 | 1 | 2 | 3 | 1 | 2 | 1 | 2 | 3 | 1 | 2 | 1 | 2 | 1 | 3 | 1 | 2 | 2 | 1 | 3 | 1 | 2 | 3 |
| 2 | 2 | 3 | 1 | 1 | 1 | 3 | 2 | 2 | 1 | 3 | 1 | 1 | 1 | 3 | 1 | 2 | 1 | 3 | 1 | 2 | 3 | 2 | 2 |
| 3 | 2 | 2 | 2 | 1 | 1 | 2 | 3 | 2 | 2 | 1 | 3 | 2 | 2 | 1 | 3 | 1 | 1 | 1 | 3 | 2 | 1 | 1 | 3 |
| 3 | 1 | 3 | 1 | 2 | 2 | 2 | 1 | 1 | 3 | 2 | 2 | 2 | 3 | 1 | 1 | 3 | 2 | 3 | 1 | 1 | 1 | 2 | 1 |
| 2 | 2 | 2 | 3 | 2 | 2 | 1 | 3 | 2 | 1 | 3 | 2 | 2 | 3 | 2 | 2 | 1 | 2 | 1 | 1 | 3 | 1 | 3 | 1 |
| 2 | 1 | 2 | 3 | 1 | 3 | 1 | 1 | 2 | 1 | 3 | 2 | 2 | 2 | 3 | 2 | 2 | 1 | 3 | 2 | 3 | 1 | 1 | 2 |
| 2 | 2 | 3 | 1 | 1 | 1 | 3 | 2 | 2 | 2 | 1 | 1 | 1 | 3 | 1 | 1 | 3 | 1 | 3 | 1 | 2 | 1 | 1 | 3 |
| 1 | 1 | 3 | 2 | 3 | 1 | 3 | 2 | 2 | 3 | 1 | 1 | 1 | 2 | 3 | 1 | 1 | 1 | 2 | 1 | 2 | 3 | 2 | 2 |
| 3 | 2 | 2 | 1 | 3 | 1 | 1 | 1 | 2 | 3 | 1 | 1 | 1 | 2 | 3 | 1 | 3 | 2 | 1 | 3 | 2 | 2 | 1 | 2 |
| 2 | 1 | 1 | 3 | 2 | 1 | 2 | 2 | 3 | 2 | 1 | 2 | 2 | 2 | 3 | 2 | 3 | 2 | 3 | 2 | 3 | 2 | 1 | 2 |
| 2 | 3 | 2 | 1 | 2 | 2 | 1 | 3 | 2 | 1 | 1 | 1 | 3 | 1 | 1 | 3 | 1 | 3 | 1 | 3 | 1 | 1 | 2 | 1 |
| 3 | 1 | 3 | 1 | 1 | 3 | 1 | 3 | 1 | 1 | 1 | 2 | 1 | 1 | 3 | 2 | 2 | 3 | 1 | 1 | 1 | 2 | 1 | 1 |
| 3 | 2 | 1 | 1 | 1 | 3 | 2 | 1 | 3 | 1 | 1 | 1 | 2 | 1 | 3 | 1 | 1 | 2 | 2 | 3 | 1 | 3 | 2 | 2 |
| 3 | 2 | 3 | 2 | 3 | 2 | 2 | 1 | 2 | 2 | 2 | 3 | 2 | 2 | 2 | 3 | 2 | 1 | 1 | 1 | 3 | 2 | 1 | 2 |
| 2 | 2 | 2 | 3 | 1 | 2 | 3 | 2 | 1 | 2 | 3 | 1 | 1 | 2 | 1 | 2 | 1 | 3 | 2 | 1 | 2 | 3 | 1 | 3 |
| 1 | 1 | 3 | 1 | 2 | 2 | 3 | 2 | 3 | 2 | 3 | 1 | 1 | 2 | 1 | 3 | 2 | 2 | 3 | 1 | 1 | 1 | 2 | 2 |
| 2 | 1 | 2 | 1 | 1 | 1 | 3 | 2 | 2 | 2 | 3 | 1 | 1 | 3 | 1 | 2 | 3 | 1 | 3 | 2 | 3 | 1 | 2 | 1 |
| 1 | 3 | 1 | 2 | 1 | 1 | 1 | 3 | 1 | 3 | 1 | 2 | 2 | 2 | 1 | 1 | 3 | 1 | 2 | 3 | 2 | 1 | 2 | 3 |
| 3 | 1 | 1 | 3 | 1 | 1 | 2 | 2 | 1 | 1 | 3 | 2 | 2 | 3 | 1 | 3 | 1 | 1 | 2 | 2 | 1 | 1 | 3 | 1 |
| 2 | 1 | 3 | 1 | 3 | 1 | 1 | 1 | 2 | 2 | 2 | 3 | 1 | 2 | 1 | 1 | 1 | 3 | 1 | 1 | 1 | 3 | 1 | 3 |
| 1 | 1 | 1 | 3 | 2 | 2 | 2 | 1 | 2 | 3 | 1 | 1 | 3 | 2 | 2 | 1 | 2 | 2 | 3 | 1 | 3 | 2 | 1 | 3 |
| 1 | 1 | 1 | 2 | 1 | 3 | 2 | 3 | 2 | 1 | 1 | 3 | 2 | 1 | 1 | 1 | 3 | 1 | 3 | 1 | 2 | 3 | 1 | 2 |
| 2 | 1 | 2 | 3 | 1 | 2 | 3 | 1 | 2 | 2 | 2 | 1 | 3 | 2 | 2 | 1 | 2 | 1 | 1 | 3 | 1 | 3 | 2 | 3 |
| 2 | 1 | 3 | 1 | 2 | 1 | 1 | 1 | 2 | 3 | 2 | 2 | 1 | 2 | 3 | 1 | 2 | 3 | 1 | 3 | 2 | 1 | 1 | 3 |
| 1 | 3 | 1 | 2 | 2 | 3 | 1 | 2 | 2 | 3 | 2 | 3 | 1 | 2 | 3 | 1 | 2 | 2 | 2 | 3 | 2 | 1 | 2 | 1 |
| 2 | 2 | 1 | 1 | 3 | 1 | 1 | 3 | 1 | 1 | 2 | 2 | 3 | 2 | 1 | 2 | 1 | 2 | 3 | 1 | 3 | 1 | 3 | 2 |
| 3 | 2 | 1 | 3 | 1 | 1 | 2 | 3 | 2 | 2 | 2 | 1 | 3 | 1 | 3 | 2 | 2 | 3 | 1 | 1 | 2 | 1 | 2 | 1 |
| 3 | 1 | 3 | 1 | 1 | 1 | 2 | 1 | 3 | 2 | 1 | 1 | 3 | 1 | 1 | 3 | 2 | 1 | 1 | 1 | 2 | 1 | 3 | 1 |
| 1 | 2 | 2 | 3 | 1 | 1 | 3 | 2 | 2 | 3 | 2 | 2 | 1 | 2 | 3 | 2 | 3 | 1 | 1 | 3 | 1 | 2 | 2 | 2 |
| 2 | 1 | 1 | 1 | 2 | 3 | 2 | 2 | 3 | 2 | 3 | 2 | 1 | 3 | 1 | 3 | 2 | 1 | 1 | 2 | 2 | 1 | 3 | 1 |
| 1 | 1 | 1 | 2 | 1 | 1 | 3 | 1 | 3 | 2 | 2 | 2 | 3 | 1 | 3 | 1 | 1 | 3 | 2 | 2 | 3 | 2 | 2 | 2 |
| 1 | 3 | 2 | 2 | 3 | 2 | 1 | 1 | 2 | 1 | 1 | 3 | 1 | 1 | 3 | 2 | 3 | 1 | 2 | 2 | 2 | 1 | 1 | 3 |
| 3 | 2 | 2 | 1 | 3 | 1 | 1 | 2 | 3 | 2 | 1 | 2 | 1 | 2 | 1 | 3 | 1 | 3 | 2 | 2 | 1 | 3 | 1 | 2 |
| 2 | 2 | 3 | 1 | 2 | 1 | 2 | 2 | 3 | 1 | 1 | 1 | 3 | 1 | 3 | 1 | 1 | 1 | 3 | 2 | 2 | 1 | 2 | 3 |
| 2 | 2 | 1 | 1 | 1 | 3 | 1 | 3 | 1 | 3 | 1 | 1 | 1 | 2 | 3 | 2 | 2 | 2 | 3 | 1 | 2 | 2 | 1 | 3 |
| 2 | 3 | 2 | 3 | 1 | 1 | 2 | 2 | 2 | 3 | 1 | 3 | 2 | 1 | 2 | 2 | 1 | 3 | 2 | 1 | 1 | 3 | 1 | 1 |
| 2 | 1 | 1 | 2 | 2 | 2 | 3 | 1 | 1 | 2 | 3 | 2 | 3 | 1 | 1 | 1 | 3 | 2 | 2 | 3 | 2 | 2 | 1 | 3 |
| 1 | 2 | 3 | 2 | 3 | 2 | 2 | 2 | 3 | 1 | 1 | 1 | 3 | 1 | 2 | 3 | 1 | 2 | 3 | 1 | 2 | 2 | 2 | 1 |
| 1 | 1 | 3 | 2 | 2 | 1 | 2 | 3 | 2 | 2 | 3 | 1 | 2 | 1 | 2 | 2 | 3 | 1 | 3 | 2 | 3 | 1 | 1 | 1 |
| 2 | 1 | 3 | 1 | 2 | 1 | 1 | 1 | 3 | 1 | 1 | 3 | 1 | 2 | 1 | 3 | 1 | 3 | 1 | 2 | 2 | 2 | 1 | 3 |
| 3 | 1 | 2 | 3 | 1 | 1 | 2 | 3 | 2 | 1 | 3 | 1 | 2 | 1 | 2 | 1 | 2 | 3 | 2 | 1 | 1 | 2 | 3 | 1 |
| 3 | 1 | 1 | 3 | 1 | 1 | 2 | 1 | 3 | 2 | 2 | 2 | 1 | 2 | 3 | 2 | 1 | 1 | 1 | 2 | 3 | 1 | 2 | 3 |
| 3 | 2 | 1 | 3 | 2 | 1 | 2 | 1 | 2 | 1 | 3 | 2 | 2 | 1 | 1 | 1 | 3 | 1 | 2 | 3 | 1 | 3 | 2 | 2 |
| 3 | 2 | 2 | 1 | 2 | 2 | 2 | 3 | 2 | 3 | 2 | 1 | 2 | 3 | 1 | 2 | 2 | 1 | 2 | 3 | 1 | 2 | 2 | 3 |
| 1 | 3 | 1 | 3 | 1 | 2 | 2 | 1 | 3 | 1 | 1 | 1 | 2 | 2 | 3 | 1 | 3 | 1 | 3 | 1 | 1 | 2 | 2 | 1 |
| 3 | 2 | 1 | 2 | 3 | 1 | 2 | 1 | 3 | 1 | 3 | 2 | 2 | 2 | 1 | 2 | 1 | 3 | 2 | 3 | 1 | 2 | 1 | 1 |
| 3 | 2 | 2 | 1 | 3 | 1 | 1 | 1 | 3 | 1 | 1 | 2 | 3 | 1 | 1 | 1 | 2 | 2 | 3 | 1 | 1 | 3 | 2 | 1 |
| 1 | 1 | 3 | 1 | 1 | 2 | 3 | 1 | 3 | 1 | 1 | 2 | 1 | 2 | 1 | 3 | 1 | 3 | 1 | 2 | 3 | 1 | 1 | 2 |
| 1 | 1 | 1 | 3 | 1 | 3 | 1 | 1 | 2 | 1 | 3 | 2 | 3 | 2 | 2 | 2 | 1 | 1 | 3 | 1 | 1 | 3 | 1 | 2 |
| 3 | 1 | 2 | 3 | 2 | 3 | 2 | 2 | 1 | 2 | 2 | 3 | 1 | 2 | 1 | 3 | 1 | 1 | 1 | 2 | 2 | 1 | 3 | 1 |
| 2 | 1 | 3 | 1 | 3 | 2 | 2 | 1 | 2 | 1 | 3 | 1 | 3 | 1 | 2 | 1 | 2 | 2 | 3 | 2 | 1 | 2 | 3 | 1 |
| 3 | 1 | 3 | 1 | 3 | 2 | 2 | 3 | 1 | 1 | 2 | 1 | 1 | 3 | 2 | 2 | 1 | 1 | 1 | 3 | 1 | 2 | 1 | 2 |
| 1 | 3 | 1 | 2 | 1 | 2 | 3 | 1 | 1 | 1 | 2 | 1 | 3 | 1 | 2 | 2 | 3 | 2 | 2 | 1 | 3 | 1 | 3 | 1 |
| 3 | 1 | 3 | 2 | 3 | 1 | 1 | 2 | 1 | 3 | 1 | 1 | 1 | 3 | 1 | 2 | 1 | 2 | 3 | 2 | 2 | 1 | 1 | 2 |
| 1 | 1 | 1 | 3 | 1 | 3 | 1 | 2 | 1 | 2 | 2 | 3 | 1 | 1 | 3 | 1 | 3 | 1 | 1 | 2 | 1 | 1 | 1 | 3 |
| 3 | 2 | 2 | 1 | 2 | 1 | 3 | 1 | 1 | 2 | 1 | 1 | 3 | 2 | 2 | 3 | 2 | 1 | 1 | 1 | 3 | 2 | 3 | 2 |
| 2 | 3 | 1 | 2 | 1 | 3 | 2 | 1 | 2 | 3 | 1 | 2 | 1 | 1 | 2 | 3 | 2 | 3 | 2 | 2 | 2 | 1 | 2 | 3 |
| 2 | 2 | 2 | 3 | 2 | 2 | 3 | 2 | 2 | 1 | 1 | 3 | 2 | 1 | 2 | 3 | 2 | 3 | 1 | 2 | 2 | 2 | 1 | 3 |
| 2 | 1 | 1 | 1 | 3 | 2 | 3 | 2 | 2 | 3 | 2 | 3 | 2 | 2 | 1 | 1 | 1 | 3 | 1 | 2 | 2 | 1 | 1 | 3 |
| 2 | 3 | 2 | 3 | 2 | 2 | 2 | 3 | 1 | 2 | 2 | 3 | 1 | 2 | 2 | 1 | 1 | 2 | 3 | 2 | 2 | 1 | 2 | 3 |
| 1 | 2 | 2 | 1 | 1 | 2 | 3 | 1 | 1 | 2 | 3 | 1 | 3 | 2 | 3 | 2 | 2 | 3 | 2 | 1 | 1 | 2 | 3 | 2 |
| 2 | 1 | 3 | 1 | 2 | 3 | 2 | 2 | 2 | 3 | 2 | 3 | 1 | 3 | 2 | 2 | 2 | 3 | 1 | 2 | 1 | 2 | 2 | 1 |
| 3 | 1 | 1 | 2 | 3 | 1 | 1 | 2 | 1 | 3 | 2 | 1 | 1 | 2 | 1 | 3 | 1 | 2 | 3 | 1 | 2 | 2 | 2 | 3 |
| 1 | 1 | 2 | 1 | 3 | 2 | 3 | 2 | 3 | 2 | 2 | 3 | 2 | 2 | 1 | 2 | 1 | 2 | 3 | 1 | 2 | 2 | 1 | 3 |
| 2 | 1 | 3 | 1 | 2 | 2 | 1 | 3 | 1 | 1 | 3 | 1 | 2 | 3 | 2 | 2 | 3 | 2 | 3 | 2 | 1 | 2 | 2 | 1 |
| 1 | 1 | 2 | 3 | 2 | 3 | 2 | 3 | 2 | 3 | 2 | 2 | 1 | 1 | 1 | 2 | 3 | 1 | 1 | 2 | 2 | 2 | 3 | 2 |
| 3 | 1 | 1 | 2 | 2 | 1 | 1 | 3 | 2 | 1 | 2 | 1 | 2 | 3 | 1 | 3 | 2 | 3 | 2 | 1 | 3 | 1 | 1 | 1 |
| 2 | 2 | 1 | 2 | 2 | 3 | 2 | 3 | 2 | 3 | 2 | 1 | 1 | 3 | 2 | 1 | 3 | 2 | 3 | 2 | 1 | 1 | 1 | 2 |
| 3 | 2 | 1 | 3 | 2 | 1 | 1 | 1 | 3 | 1 | 3 | 1 | 1 | 2 | 2 | 3 | 2 | 2 | 2 | 1 | 3 | 2 | 1 | 2 |
| 1 | 1 | 3 | 2 | 2 | 2 | 3 | 2 | 1 | 1 | 3 | 1 | 1 | 3 | 2 | 1 | 3 | 2 | 2 | 3 | 1 | 1 | 2 | 1 |
| 1 | 3 | 2 | 2 | 1 | 2 | 1 | 3 | 2 | 1 | 2 | 1 | 3 | 2 | 1 | 3 | 2 | 1 | 2 | 1 | 3 | 1 | 3 | 1 |
| 3 | 1 | 1 | 1 | 3 | 1 | 1 | 1 | 2 | 3 | 2 | 3 | 2 | 1 | 2 | 1 | 3 | 2 | 2 | 2 | 1 | 1 | 2 | 3 |
| 2 | 2 | 3 | 2 | 3 | 1 | 3 | 2 | 1 | 1 | 2 | 3 | 1 | 1 | 2 | 3 | 1 | 2 | 3 | 2 | 1 | 2 | 2 | 1 |
| 3 | 2 | 1 | 3 | 1 | 3 | 2 | 2 | 3 | 2 | 1 | 1 | 1 | 2 | 1 | 3 | 1 | 3 | 1 | 1 | 2 | 1 | 1 | 1 |
| 1 | 2 | 2 | 1 | 1 | 2 | 3 | 2 | 1 | 3 | 1 | 2 | 2 | 3 | 2 | 1 | 1 | 3 | 1 | 3 | 1 | 2 | 1 | 3 |
| 2 | 2 | 1 | 3 | 2 | 3 | 2 | 3 | 2 | 2 | 2 | 3 | 2 | 1 | 3 | 1 | 2 | 1 | 3 | 1 | 1 | 2 | 2 | 1 |
| 1 | 3 | 1 | 3 | 1 | 3 | 2 | 2 | 2 | 3 | 2 | 3 | 2 | 1 | 2 | 1 | 2 | 3 | 2 | 1 | 2 | 1 | 1 | 1 |
| 2 | 2 | 1 | 1 | 3 | 2 | 2 | 2 | 1 | 3 | 2 | 3 | 1 | 3 | 1 | 2 | 2 | 2 | 3 | 2 | 2 | 1 | 1 | 3 |
| 1 | 2 | 3 | 1 | 1 | 3 | 2 | 2 | 2 | 1 | 2 | 2 | 3 | 1 | 1 | 2 | 1 | 3 | 2 | 1 | 3 | 2 | 3 | 1 |
| 1 | 2 | 1 | 2 | 2 | 2 | 3 | 2 | 3 | 2 | 2 | 3 | 2 | 1 | 2 | 3 | 2 | 2 | 2 | 3 | 2 | 3 | 1 | 1 |
| 1 | 1 | 1 | 3 | 2 | 3 | 2 | 2 | 2 | 1 | 2 | 1 | 3 | 1 | 1 | 3 | 1 | 2 | 2 | 2 | 3 | 1 | 2 | 3 |
| 1 | 1 | 3 | 1 | 3 | 1 | 2 | 1 | 2 | 3 | 1 | 2 | 2 | 2 | 3 | 2 | 2 | 1 | 3 | 2 | 2 | 3 | 2 | 1 |
| 1 | 1 | 3 | 1 | 1 | 3 | 1 | 1 | 1 | 2 | 3 | 1 | 3 | 2 | 3 | 1 | 2 | 1 | 1 | 2 | 3 | 2 | 1 | 1 |
| 2 | 1 | 3 | 2 | 3 | 2 | 2 | 2 | 3 | 1 | 2 | 1 | 2 | 3 | 2 | 2 | 1 | 1 | 3 | 1 | 1 | 3 | 2 | 2 |
| 3 | 2 | 1 | 3 | 1 | 1 | 1 | 3 | 2 | 3 | 1 | 2 | 1 | 3 | 1 | 2 | 2 | 1 | 3 | 2 | 1 | 1 | 2 | 1 |
| 3 | 1 | 2 | 1 | 1 | 1 | 2 | 3 | 2 | 2 | 1 | 1 | 3 | 2 | 2 | 1 | 3 | 2 | 1 | 2 | 3 | 1 | 2 | 3 |
| 1 | 3 | 1 | 2 | 2 | 1 | 3 | 1 | 1 | 3 | 1 | 1 | 2 | 2 | 3 | 2 | 2 | 2 | 1 | 3 | 1 | 1 | 2 | 3 |
| 1 | 2 | 1 | 2 | 2 | 2 | 3 | 1 | 3 | 1 | 1 | 3 | 2 | 3 | 2 | 3 | 1 | 1 | 1 | 2 | 3 | 1 | 1 | 2 |
| 2 | 3 | 1 | 3 | 2 | 1 | 1 | 1 | 2 | 1 | 3 | 2 | 2 | 2 | 1 | 2 | 3 | 1 | 3 | 2 | 1 | 3 | 2 | 1 |
| 2 | 2 | 1 | 3 | 1 | 3 | 1 | 3 | 2 | 1 | 3 | 1 | 2 | 1 | 1 | 1 | 3 | 1 | 2 | 2 | 2 | 3 | 1 | 2 |
| 1 | 2 | 2 | 3 | 2 | 2 | 2 | 1 | 1 | 3 | 2 | 2 | 3 | 2 | 2 | 3 | 1 | 2 | 1 | 1 | 3 | 1 | 2 | 3 |
| 3 | 2 | 2 | 3 | 2 | 1 | 1 | 1 | 3 | 2 | 2 | 1 | 1 | 1 | 3 | 2 | 3 | 2 | 3 | 1 | 1 | 2 | 2 | 2 |
| 1 | 2 | 1 | 3 | 1 | 2 | 2 | 3 | 2 | 3 | 2 | 3 | 2 | 2 | 2 | 3 | 2 | 2 | 1 | 2 | 1 | 3 | 2 | 1 |
| 3 | 2 | 1 | 1 | 3 | 2 | 2 | 1 | 2 | 2 | 3 | 1 | 3 | 1 | 1 | 2 | 3 | 1 | 2 | 1 | 1 | 2 | 1 | 3 |
| 2 | 1 | 3 | 1 | 2 | 2 | 1 | 3 | 2 | 2 | 3 | 1 | 2 | 1 | 1 | 3 | 2 | 3 | 2 | 3 | 2 | 1 | 1 | 2 |
| 1 | 1 | 1 | 2 | 3 | 2 | 1 | 1 | 1 | 2 | 3 | 1 | 1 | 3 | 1 | 3 | 2 | 3 | 2 | 2 | 2 | 3 | 2 | 2 |
| 3 | 1 | 2 | 1 | 3 | 1 | 1 | 3 | 1 | 1 | 1 | 2 | 3 | 2 | 1 | 2 | 1 | 2 | 1 | 3 | 2 | 3 | 1 | 2 |
| 2 | 1 | 2 | 1 | 3 | 1 | 3 | 2 | 3 | 2 | 1 | 2 | 3 | 2 | 2 | 1 | 2 | 3 | 1 | 2 | 1 | 1 | 1 | 3 |
| 2 | 1 | 2 | 3 | 1 | 1 | 3 | 2 | 3 | 1 | 2 | 1 | 1 | 3 | 1 | 2 | 3 | 1 | 1 | 3 | 1 | 1 | 2 | 2 |
| 2 | 3 | 2 | 2 | 3 | 1 | 3 | 1 | 1 | 2 | 1 | 3 | 2 | 1 | 1 | 3 | 1 | 3 | 1 | 1 | 2 | 2 | 2 | 1 |
| 2 | 1 | 3 | 1 | 2 | 1 | 1 | 2 | 3 | 2 | 3 | 1 | 1 | 3 | 2 | 1 | 1 | 2 | 1 | 1 | 3 | 2 | 3 | 1 |
| 3 | 2 | 1 | 2 | 2 | 1 | 2 | 3 | 1 | 2 | 3 | 1 | 2 | 1 | 3 | 2 | 1 | 3 | 2 | 1 | 1 | 3 | 2 | 1 |
| 1 | 3 | 1 | 2 | 1 | 2 | 3 | 1 | 2 | 2 | 2 | 1 | 3 | 2 | 1 | 2 | 2 | 3 | 1 | 1 | 2 | 3 | 2 | 3 |
| 1 | 1 | 2 | 2 | 1 | 1 | 3 | 2 | 2 | 2 | 3 | 2 | 1 | 3 | 1 | 3 | 2 | 3 | 1 | 2 | 2 | 2 | 3 | 1 |
| 1 | 1 | 3 | 1 | 1 | 1 | 2 | 1 | 3 | 1 | 2 | 3 | 2 | 1 | 3 | 2 | 1 | 1 | 3 | 1 | 2 | 3 | 2 | 2 |
| 2 | 2 | 3 | 1 | 3 | 1 | 1 | 3 | 2 | 2 | 3 | 2 | 2 | 3 | 2 | 1 | 1 | 2 | 1 | 1 | 3 | 1 | 1 | 2 |
| 1 | 3 | 2 | 3 | 2 | 3 | 1 | 1 | 1 | 2 | 1 | 3 | 2 | 3 | 1 | 1 | 1 | 3 | 2 | 2 | 2 | 1 | 1 | 1 |
| 2 | 2 | 2 | 1 | 2 | 3 | 2 | 1 | 3 | 2 | 1 | 3 | 1 | 2 | 2 | 2 | 1 | 2 | 3 | 2 | 3 | 1 | 1 | 3 |
| 1 | 2 | 2 | 1 | 1 | 2 | 3 | 1 | 3 | 1 | 1 | 1 | 2 | 2 | 1 | 3 | 2 | 3 | 2 | 3 | 2 | 2 | 1 | 3 |
| 1 | 2 | 3 | 2 | 2 | 1 | 1 | 2 | 1 | 3 | 2 | 3 | 1 | 2 | 1 | 3 | 2 | 1 | 1 | 1 | 3 | 2 | 3 | 1 |
| 2 | 1 | 2 | 3 | 2 | 2 | 3 | 1 | 2 | 1 | 1 | 1 | 2 | 3 | 1 | 2 | 2 | 1 | 2 | 3 | 1 | 3 | 2 | 3 |
| 2 | 2 | 1 | 2 | 2 | 1 | 3 | 1 | 3 | 2 | 2 | 3 | 2 | 3 | 2 | 3 | 2 | 3 | 1 | 2 | 1 | 2 | 1 | 2 |
| 2 | 3 | 2 | 2 | 3 | 2 | 2 | 1 | 2 | 3 | 1 | 2 | 2 | 3 | 1 | 3 | 2 | 2 | 1 | 3 | 1 | 1 | 2 | 1 |
| 1 | 1 | 2 | 2 | 2 | 3 | 1 | 3 | 2 | 2 | 1 | 1 | 3 | 1 | 1 | 3 | 1 | 1 | 3 | 2 | 3 | 2 | 1 | 1 |
| 1 | 1 | 1 | 3 | 1 | 2 | 1 | 1 | 1 | 3 | 2 | 2 | 1 | 1 | 3 | 2 | 3 | 2 | 2 | 2 | 3 | 2 | 1 | 3 |
| 2 | 3 | 2 | 2 | 3 | 1 | 3 | 1 | 2 | 3 | 1 | 2 | 1 | 2 | 2 | 3 | 2 | 1 | 2 | 1 | 1 | 3 | 2 | 2 |
| 2 | 1 | 1 | 1 | 2 | 1 | 3 | 2 | 3 | 1 | 1 | 2 | 3 | 1 | 3 | 2 | 2 | 1 | 2 | 1 | 3 | 1 | 3 | 2 |
| 1 | 2 | 1 | 3 | 1 | 2 | 3 | 2 | 2 | 1 | 2 | 3 | 1 | 2 | 1 | 3 | 2 | 2 | 1 | 3 | 2 | 2 | 1 | 3 |
| 3 | 2 | 2 | 1 | 1 | 3 | 2 | 3 | 1 | 1 | 3 | 1 | 2 | 1 | 2 | 3 | 2 | 1 | 2 | 2 | 3 | 2 | 2 | 1 |
| 2 | 1 | 1 | 3 | 1 | 1 | 1 | 3 | 2 | 1 | 1 | 1 | 3 | 2 | 2 | 2 | 3 | 2 | 1 | 3 | 1 | 2 | 3 | 2 |
| 1 | 1 | 3 | 1 | 3 | 1 | 1 | 1 | 3 | 2 | 2 | 2 | 3 | 1 | 2 | 2 | 3 | 1 | 1 | 2 | 1 | 1 | 1 | 3 |
| 1 | 2 | 1 | 2 | 2 | 1 | 3 | 1 | 2 | 3 | 2 | 3 | 1 | 3 | 2 | 2 | 1 | 2 | 1 | 2 | 3 | 2 | 3 | 2 |
| 1 | 3 | 2 | 2 | 2 | 3 | 1 | 3 | 2 | 2 | 2 | 1 | 3 | 2 | 1 | 2 | 2 | 3 | 2 | 3 | 1 | 1 | 2 | 1 |
| 1 | 2 | 3 | 2 | 2 | 1 | 1 | 1 | 2 | 3 | 1 | 3 | 1 | 3 | 1 | 2 | 2 | 3 | 2 | 3 | 2 | 1 | 2 | 1 |
| 2 | 1 | 1 | 1 | 2 | 3 | 2 | 2 | 3 | 2 | 3 | 1 | 2 | 2 | 1 | 2 | 2 | 3 | 2 | 3 | 1 | 3 | 1 | 2 |
| 2 | 1 | 1 | 3 | 1 | 1 | 2 | 2 | 3 | 1 | 1 | 3 | 2 | 1 | 1 | 3 | 1 | 3 | 2 | 2 | 1 | 2 | 2 | 3 |
| 1 | 3 | 1 | 3 | 1 | 2 | 1 | 3 | 1 | 1 | 2 | 2 | 1 | 1 | 3 | 2 | 2 | 2 | 3 | 2 | 2 | 3 | 1 | 2 |
| 3 | 1 | 1 | 3 | 1 | 1 | 2 | 3 | 2 | 2 | 1 | 1 | 3 | 1 | 1 | 1 | 2 | 1 | 2 | 3 | 2 | 1 | 1 | 3 |
| 2 | 1 | 2 | 2 | 2 | 3 | 2 | 3 | 1 | 2 | 2 | 1 | 1 | 3 | 1 | 1 | 3 | 2 | 2 | 3 | 1 | 3 | 1 | 1 |
| 1 | 3 | 2 | 2 | 1 | 3 | 1 | 1 | 2 | 2 | 2 | 3 | 2 | 3 | 2 | 1 | 3 | 2 | 1 | 3 | 1 | 1 | 2 | 2 |
| 1 | 1 | 3 | 2 | 2 | 2 | 1 | 2 | 2 | 3 | 2 | 2 | 3 | 1 | 2 | 3 | 2 | 2 | 3 | 2 | 1 | 2 | 2 | 3 |
| 3 | 1 | 1 | 2 | 3 | 1 | 3 | 2 | 2 | 2 | 1 | 1 | 3 | 1 | 3 | 2 | 2 | 2 | 1 | 2 | 1 | 3 | 2 | 1 |
| 1 | 3 | 2 | 3 | 1 | 1 | 3 | 1 | 2 | 2 | 3 | 2 | 1 | 2 | 3 | 2 | 1 | 3 | 2 | 1 | 2 | 1 | 1 | 1 |
| 1 | 3 | 2 | 2 | 3 | 1 | 1 | 1 | 2 | 3 | 1 | 3 | 2 | 1 | 2 | 2 | 1 | 1 | 3 | 2 | 1 | 1 | 2 | 3 |
| 1 | 2 | 3 | 2 | 3 | 2 | 2 | 1 | 2 | 2 | 2 | 3 | 1 | 3 | 1 | 2 | 3 | 1 | 3 | 2 | 1 | 1 | 2 | 2 |
| 1 | 1 | 1 | 2 | 1 | 3 | 2 | 3 | 2 | 2 | 3 | 2 | 2 | 3 | 1 | 1 | 3 | 2 | 2 | 3 | 2 | 2 | 1 | 2 |
| 3 | 2 | 1 | 3 | 1 | 3 | 1 | 1 | 1 | 3 | 1 | 2 | 1 | 2 | 1 | 2 | 3 | 2 | 1 | 3 | 2 | 2 | 2 | 1 |
| 3 | 1 | 3 | 1 | 3 | 2 | 1 | 2 | 2 | 2 | 3 | 1 | 2 | 3 | 1 | 1 | 2 | 3 | 1 | 2 | 2 | 1 | 2 | 1 |
| 3 | 1 | 3 | 2 | 1 | 2 | 1 | 1 | 3 | 2 | 2 | 2 | 1 | 3 | 2 | 3 | 2 | 1 | 2 | 1 | 2 | 2 | 3 | 1 |
| 1 | 2 | 1 | 1 | 2 | 3 | 2 | 3 | 1 | 2 | 2 | 1 | 2 | 2 | 3 | 1 | 2 | 2 | 3 | 1 | 3 | 1 | 3 | 1 |
| 2 | 2 | 1 | 3 | 2 | 2 | 3 | 2 | 2 | 1 | 2 | 3 | 2 | 3 | 1 | 3 | 1 | 3 | 2 | 1 | 1 | 2 | 1 | 1 |
| 1 | 1 | 1 | 2 | 3 | 1 | 3 | 2 | 1 | 2 | 1 | 2 | 2 | 3 | 1 | 1 | 2 | 2 | 3 | 2 | 3 | 1 | 2 | 3 |
| 1 | 1 | 2 | 2 | 1 | 3 | 1 | 1 | 3 | 2 | 1 | 1 | 3 | 2 | 1 | 3 | 1 | 3 | 2 | 2 | 2 | 1 | 1 | 3 |
| 2 | 3 | 2 | 1 | 1 | 3 | 2 | 2 | 2 | 1 | 1 | 1 | 3 | 2 | 1 | 1 | 3 | 1 | 1 | 1 | 2 | 3 | 2 | 3 |
| 3 | 1 | 1 | 1 | 2 | 3 | 1 | 2 | 1 | 1 | 3 | 2 | 2 | 3 | 1 | 2 | 1 | 2 | 1 | 1 | 3 | 1 | 1 | 3 |
| 1 | 1 | 2 | 3 | 1 | 3 | 2 | 1 | 3 | 2 | 2 | 2 | 3 | 2 | 1 | 2 | 2 | 2 | 3 | 1 | 3 | 2 | 2 | 2 |
| 1 | 3 | 2 | 3 | 1 | 1 | 2 | 3 | 2 | 1 | 1 | 3 | 1 | 2 | 2 | 1 | 2 | 3 | 2 | 1 | 2 | 2 | 2 | 3 |
| 3 | 2 | 1 | 1 | 2 | 2 | 3 | 1 | 1 | 2 | 2 | 3 | 1 | 1 | 1 | 3 | 1 | 2 | 1 | 1 | 3 | 2 | 3 | 2 |
| 2 | 1 | 2 | 3 | 2 | 2 | 2 | 1 | 1 | 3 | 2 | 1 | 3 | 2 | 3 | 1 | 1 | 1 | 2 | 1 | 3 | 1 | 3 | 2 |
| 3 | 2 | 1 | 2 | 2 | 3 | 1 | 1 | 1 | 2 | 2 | 3 | 1 | 1 | 2 | 2 | 1 | 3 | 1 | 1 | 3 | 2 | 1 | 3 |
| 1 | 1 | 2 | 1 | 2 | 3 | 2 | 1 | 1 | 2 | 3 | 2 | 1 | 3 | 2 | 2 | 3 | 1 | 1 | 1 | 3 | 2 | 3 | 1 |
| 2 | 3 | 1 | 1 | 2 | 1 | 2 | 2 | 3 | 1 | 3 | 1 | 1 | 2 | 2 | 1 | 2 | 3 | 1 | 3 | 1 | 3 | 2 | 2 |
| 2 | 1 | 3 | 2 | 3 | 2 | 1 | 1 | 1 | 2 | 3 | 1 | 2 | 3 | 1 | 1 | 3 | 1 | 1 | 1 | 3 | 2 | 1 | 2 |
| 3 | 2 | 1 | 2 | 3 | 2 | 3 | 2 | 1 | 1 | 1 | 3 | 1 | 1 | 1 | 2 | 2 | 2 | 3 | 1 | 2 | 3 | 2 | 1 |
| 1 | 1 | 2 | 2 | 3 | 2 | 2 | 2 | 3 | 1 | 1 | 1 | 3 | 2 | 2 | 2 | 3 | 2 | 2 | 3 | 1 | 3 | 1 | 1 |
| 1 | 1 | 2 | 2 | 3 | 2 | 2 | 2 | 3 | 1 | 3 | 2 | 1 | 3 | 2 | 1 | 2 | 2 | 1 | 3 | 2 | 1 | 3 | 2 |
| 2 | 1 | 1 | 2 | 2 | 3 | 1 | 3 | 2 | 2 | 2 | 3 | 1 | 1 | 2 | 1 | 1 | 3 | 1 | 3 | 1 | 3 | 2 | 2 |
| 2 | 3 | 2 | 2 | 3 | 1 | 2 | 2 | 3 | 2 | 1 | 1 | 3 | 2 | 3 | 2 | 2 | 2 | 1 | 2 | 2 | 3 | 2 | 2 |
| 3 | 1 | 1 | 1 | 2 | 2 | 2 | 3 | 2 | 3 | 1 | 3 | 2 | 1 | 2 | 3 | 2 | 1 | 2 | 2 | 2 | 3 | 1 | 1 |
| 2 | 1 | 1 | 3 | 1 | 1 | 2 | 3 | 1 | 1 | 2 | 3 | 2 | 3 | 1 | 1 | 3 | 2 | 3 | 1 | 1 | 2 | 1 | 2 |
| 2 | 1 | 1 | 2 | 3 | 2 | 3 | 1 | 1 | 3 | 2 | 2 | 2 | 3 | 2 | 3 | 1 | 1 | 1 | 3 | 1 | 2 | 1 | 2 |
| 2 | 2 | 3 | 2 | 1 | 2 | 1 | 2 | 3 | 1 | 1 | 1 | 3 | 2 | 1 | 1 | 3 | 1 | 1 | 3 | 1 | 1 | 3 | 2 |
| 2 | 1 | 3 | 1 | 3 | 1 | 3 | 1 | 1 | 3 | 1 | 1 | 3 | 1 | 1 | 1 | 2 | 1 | 1 | 3 | 1 | 1 | 2 | 1 |
| 1 | 2 | 2 | 2 | 3 | 1 | 1 | 1 | 2 | 3 | 2 | 2 | 1 | 1 | 2 | 3 | 1 | 3 | 1 | 3 | 1 | 3 | 1 | 2 |
| 2 | 2 | 3 | 2 | 3 | 2 | 3 | 2 | 1 | 2 | 1 | 2 | 1 | 3 | 2 | 1 | 2 | 2 | 1 | 3 | 1 | 1 | 2 | 3 |
| 1 | 2 | 2 | 3 | 2 | 2 | 1 | 3 | 2 | 1 | 2 | 2 | 3 | 1 | 2 | 3 | 2 | 3 | 1 | 1 | 3 | 2 | 2 | 1 |
| 2 | 3 | 2 | 2 | 2 | 3 | 2 | 1 | 2 | 2 | 2 | 3 | 1 | 1 | 2 | 3 | 1 | 1 | 1 | 2 | 3 | 1 | 1 | 3 |
| 2 | 3 | 2 | 2 | 1 | 3 | 1 | 2 | 2 | 3 | 2 | 3 | 2 | 2 | 1 | 1 | 1 | 2 | 3 | 2 | 1 | 3 | 2 | 2 |
| 2 | 1 | 2 | 1 | 3 | 1 | 3 | 2 | 1 | 2 | 2 | 3 | 2 | 1 | 2 | 1 | 3 | 1 | 3 | 1 | 3 | 1 | 1 | 1 |
| 1 | 1 | 1 | 2 | 1 | 3 | 2 | 1 | 1 | 3 | 1 | 1 | 2 | 3 | 2 | 1 | 3 | 2 | 2 | 3 | 2 | 2 | 3 | 1 |
| 2 | 3 | 1 | 3 | 2 | 3 | 2 | 3 | 1 | 2 | 2 | 2 | 1 | 2 | 3 | 1 | 2 | 2 | 1 | 1 | 3 | 2 | 2 | 1 |
| 1 | 3 | 1 | 1 | 2 | 2 | 2 | 3 | 2 | 2 | 3 | 2 | 1 | 3 | 2 | 3 | 2 | 2 | 1 | 2 | 3 | 1 | 2 | 2 |
| 3 | 1 | 2 | 2 | 3 | 1 | 1 | 3 | 1 | 1 | 1 | 3 | 1 | 1 | 1 | 2 | 1 | 3 | 2 | 2 | 2 | 3 | 1 | 1 |
| 3 | 1 | 2 | 1 | 1 | 2 | 1 | 3 | 1 | 3 | 1 | 1 | 2 | 1 | 3 | 2 | 1 | 3 | 1 | 3 | 2 | 2 | 1 | 1 |
| 3 | 1 | 2 | 2 | 3 | 1 | 1 | 1 | 2 | 2 | 2 | 3 | 2 | 1 | 3 | 2 | 2 | 1 | 2 | 1 | 3 | 2 | 3 | 1 |
| 3 | 1 | 2 | 2 | 2 | 1 | 1 | 3 | 1 | 1 | 3 | 1 | 2 | 3 | 1 | 1 | 2 | 1 | 1 | 2 | 3 | 2 | 1 | 3 |
| 2 | 2 | 2 | 3 | 1 | 3 | 1 | 3 | 1 | 1 | 1 | 3 | 2 | 1 | 3 | 1 | 1 | 2 | 1 | 1 | 3 | 1 | 2 | 1 |
| 3 | 1 | 2 | 2 | 1 | 1 | 3 | 1 | 3 | 2 | 1 | 1 | 1 | 2 | 3 | 1 | 3 | 2 | 1 | 2 | 1 | 1 | 3 | 1 |
| 2 | 2 | 2 | 3 | 1 | 2 | 1 | 3 | 1 | 1 | 2 | 2 | 3 | 1 | 1 | 1 | 2 | 2 | 2 | 3 | 1 | 3 | 1 | 3 |
| 2 | 3 | 1 | 1 | 3 | 1 | 1 | 3 | 1 | 3 | 2 | 3 | 2 | 2 | 1 | 2 | 1 | 1 | 3 | 1 | 2 | 2 | 2 | 1 |
| 3 | 2 | 3 | 1 | 1 | 1 | 2 | 3 | 1 | 2 | 2 | 2 | 1 | 3 | 1 | 3 | 2 | 1 | 1 | 2 | 1 | 1 | 3 | 2 |
| 1 | 1 | 1 | 2 | 1 | 1 | 3 | 1 | 1 | 2 | 1 | 3 | 1 | 3 | 1 | 3 | 1 | 3 | 1 | 1 | 1 | 3 | 2 | 2 |
| 3 | 2 | 2 | 3 | 2 | 1 | 1 | 1 | 3 | 2 | 1 | 1 | 2 | 1 | 3 | 1 | 3 | 1 | 1 | 1 | 2 | 2 | 1 | 3 |
| 1 | 3 | 2 | 3 | 1 | 2 | 2 | 2 | 1 | 3 | 1 | 2 | 2 | 1 | 2 | 3 | 2 | 3 | 1 | 2 | 3 | 1 | 2 | 2 |
| 1 | 3 | 1 | 3 | 2 | 1 | 2 | 1 | 3 | 2 | 2 | 2 | 1 | 3 | 1 | 2 | 2 | 2 | 1 | 2 | 3 | 2 | 1 | 3 |
| 1 | 2 | 3 | 1 | 2 | 2 | 1 | 3 | 1 | 2 | 1 | 3 | 2 | 3 | 1 | 1 | 1 | 2 | 2 | 3 | 2 | 2 | 1 | 3 |
| 1 | 2 | 3 | 1 | 1 | 1 | 2 | 3 | 2 | 1 | 2 | 2 | 1 | 3 | 2 | 2 | 2 | 1 | 3 | 1 | 3 | 2 | 2 | 3 |
| 1 | 2 | 1 | 2 | 2 | 3 | 1 | 3 | 2 | 3 | 1 | 3 | 1 | 3 | 2 | 2 | 1 | 2 | 2 | 3 | 2 | 2 | 1 | 1 |
| 1 | 3 | 1 | 2 | 3 | 2 | 3 | 2 | 1 | 2 | 2 | 3 | 1 | 1 | 2 | 2 | 1 | 1 | 3 | 1 | 1 | 3 | 2 | 2 |
| 2 | 1 | 1 | 2 | 3 | 2 | 3 | 2 | 2 | 3 | 1 | 2 | 1 | 3 | 1 | 1 | 2 | 1 | 3 | 1 | 3 | 1 | 2 | 1 |
| 1 | 1 | 1 | 2 | 2 | 1 | 3 | 2 | 2 | 3 | 1 | 1 | 1 | 3 | 2 | 1 | 2 | 3 | 1 | 3 | 1 | 1 | 1 | 3 |
| 2 | 2 | 2 | 1 | 3 | 1 | 3 | 2 | 2 | 3 | 1 | 1 | 3 | 1 | 1 | 1 | 2 | 3 | 2 | 2 | 1 | 1 | 2 | 3 |
| 3 | 1 | 2 | 2 | 3 | 2 | 2 | 3 | 1 | 2 | 2 | 1 | 2 | 2 | 3 | 1 | 2 | 3 | 1 | 1 | 2 | 2 | 2 | 3 |
| 2 | 3 | 2 | 2 | 3 | 2 | 2 | 3 | 2 | 2 | 3 | 1 | 1 | 2 | 2 | 3 | 1 | 1 | 3 | 1 | 1 | 2 | 2 | 1 |
| 1 | 2 | 2 | 1 | 1 | 3 | 2 | 1 | 1 | 3 | 1 | 1 | 2 | 2 | 3 | 1 | 3 | 1 | 3 | 2 | 2 | 2 | 3 | 1 |
| 3 | 2 | 1 | 2 | 3 | 2 | 2 | 3 | 2 | 1 | 1 | 2 | 3 | 2 | 1 | 2 | 2 | 1 | 1 | 3 | 1 | 1 | 1 | 3 |
| 2 | 1 | 3 | 2 | 2 | 3 | 2 | 3 | 1 | 2 | 2 | 2 | 1 | 2 | 3 | 2 | 1 | 1 | 2 | 3 | 1 | 2 | 2 | 3 |
| 2 | 3 | 1 | 2 | 1 | 1 | 2 | 3 | 1 | 1 | 1 | 3 | 2 | 2 | 2 | 1 | 2 | 1 | 3 | 1 | 3 | 1 | 3 | 1 |
| 3 | 2 | 1 | 1 | 3 | 1 | 2 | 2 | 3 | 2 | 2 | 2 | 3 | 2 | 1 | 2 | 3 | 1 | 2 | 1 | 1 | 3 | 1 | 2 |
| 2 | 2 | 3 | 1 | 1 | 2 | 2 | 1 | 1 | 3 | 1 | 3 | 2 | 1 | 1 | 3 | 1 | 2 | 3 | 2 | 2 | 2 | 1 | 3 |
| 1 | 1 | 3 | 2 | 3 | 2 | 2 | 2 | 3 | 2 | 2 | 2 | 1 | 3 | 1 | 3 | 2 | 1 | 1 | 1 | 3 | 1 | 2 | 1 |
| 1 | 3 | 1 | 3 | 1 | 3 | 1 | 2 | 1 | 1 | 1 | 3 | 2 | 1 | 2 | 1 | 3 | 1 | 1 | 3 | 2 | 2 | 1 | 1 |
| 1 | 2 | 2 | 1 | 2 | 3 | 1 | 1 | 2 | 1 | 3 | 2 | 2 | 1 | 3 | 1 | 1 | 1 | 3 | 1 | 3 | 1 | 3 | 2 |
| 2 | 2 | 3 | 2 | 2 | 3 | 1 | 2 | 1 | 2 | 2 | 1 | 3 | 1 | 3 | 1 | 1 | 2 | 3 | 2 | 3 | 2 | 2 | 2 |
| 2 | 2 | 2 | 1 | 2 | 2 | 3 | 1 | 3 | 1 | 3 | 2 | 2 | 2 | 3 | 2 | 2 | 1 | 2 | 2 | 2 | 3 | 2 | 3 |
| 1 | 3 | 2 | 3 | 2 | 2 | 1 | 1 | 3 | 1 | 1 | 3 | 2 | 2 | 3 | 1 | 2 | 2 | 1 | 2 | 2 | 3 | 1 | 2 |
| 3 | 1 | 3 | 1 | 1 | 1 | 2 | 3 | 1 | 2 | 2 | 3 | 1 | 1 | 2 | 3 | 2 | 2 | 3 | 1 | 2 | 1 | 1 | 2 |
| 3 | 1 | 2 | 1 | 1 | 3 | 2 | 1 | 2 | 2 | 1 | 3 | 2 | 1 | 2 | 3 | 1 | 3 | 2 | 3 | 2 | 1 | 1 | 2 |
| 2 | 2 | 2 | 3 | 1 | 2 | 2 | 2 | 1 | 1 | 3 | 1 | 3 | 2 | 3 | 2 | 2 | 3 | 1 | 1 | 2 | 3 | 2 | 1 |
| 1 | 1 | 3 | 2 | 2 | 1 | 3 | 2 | 1 | 1 | 1 | 2 | 1 | 3 | 1 | 3 | 2 | 1 | 3 | 1 | 1 | 1 | 3 | 1 |
| 3 | 2 | 1 | 1 | 1 | 3 | 2 | 1 | 2 | 3 | 1 | 1 | 2 | 1 | 2 | 3 | 2 | 3 | 1 | 1 | 1 | 2 | 3 | 2 |
| 2 | 1 | 1 | 2 | 1 | 1 | 3 | 2 | 3 | 2 | 3 | 2 | 2 | 3 | 2 | 3 | 1 | 1 | 2 | 3 | 2 | 1 | 1 | 2 |
| 1 | 1 | 1 | 3 | 1 | 2 | 2 | 2 | 1 | 3 | 1 | 3 | 2 | 1 | 3 | 1 | 1 | 1 | 3 | 1 | 3 | 1 | 1 | 2 |
| 2 | 2 | 1 | 3 | 2 | 2 | 2 | 3 | 1 | 3 | 2 | 2 | 3 | 1 | 1 | 1 | 2 | 1 | 3 | 2 | 1 | 1 | 1 | 3 |
| 2 | 1 | 1 | 2 | 1 | 3 | 2 | 1 | 2 | 3 | 1 | 3 | 2 | 1 | 1 | 3 | 1 | 2 | 2 | 3 | 1 | 1 | 1 | 3 |
| 3 | 1 | 1 | 3 | 1 | 2 | 2 | 1 | 3 | 1 | 2 | 2 | 3 | 1 | 2 | 3 | 2 | 2 | 1 | 3 | 2 | 2 | 1 | 1 |
| 2 | 1 | 1 | 1 | 3 | 1 | 3 | 1 | 3 | 1 | 1 | 3 | 2 | 2 | 1 | 3 | 2 | 1 | 1 | 2 | 1 | 3 | 1 | 1 |
| 2 | 1 | 1 | 3 | 2 | 1 | 2 | 3 | 1 | 3 | 1 | 1 | 1 | 2 | 3 | 1 | 2 | 3 | 2 | 3 | 2 | 2 | 1 | 2 |
| 3 | 1 | 3 | 2 | 2 | 2 | 3 | 2 | 2 | 2 | 3 | 2 | 2 | 1 | 3 | 2 | 2 | 1 | 2 | 2 | 3 | 1 | 2 | 1 |
| 1 | 1 | 3 | 2 | 1 | 1 | 1 | 3 | 1 | 1 | 1 | 2 | 3 | 2 | 2 | 1 | 1 | 3 | 1 | 3 | 2 | 1 | 3 | 2 |
| 1 | 2 | 3 | 1 | 3 | 1 | 1 | 2 | 2 | 3 | 2 | 2 | 3 | 2 | 2 | 3 | 1 | 1 | 1 | 2 | 3 | 2 | 1 | 1 |
| 2 | 2 | 1 | 3 | 1 | 2 | 2 | 1 | 3 | 1 | 3 | 2 | 1 | 3 | 2 | 1 | 3 | 1 | 1 | 3 | 1 | 1 | 2 | 1 |
| 2 | 1 | 3 | 2 | 3 | 1 | 2 | 3 | 1 | 1 | 3 | 1 | 1 | 3 | 2 | 2 | 1 | 3 | 1 | 1 | 1 | 2 | 2 | 1 |
| 2 | 1 | 1 | 2 | 3 | 2 | 1 | 3 | 2 | 1 | 1 | 2 | 3 | 2 | 3 | 1 | 2 | 3 | 1 | 2 | 1 | 1 | 3 | 2 |
| 2 | 2 | 3 | 1 | 3 | 1 | 1 | 1 | 3 | 1 | 1 | 2 | 1 | 1 | 3 | 2 | 1 | 3 | 2 | 3 | 2 | 1 | 1 | 1 |
| 2 | 1 | 1 | 2 | 3 | 1 | 3 | 2 | 3 | 1 | 3 | 1 | 2 | 2 | 1 | 2 | 1 | 3 | 1 | 2 | 2 | 3 | 2 | 2 |
| 3 | 2 | 1 | 2 | 1 | 1 | 3 | 1 | 1 | 1 | 2 | 3 | 2 | 3 | 2 | 3 | 2 | 2 | 2 | 3 | 1 | 2 | 2 | 1 |
| 3 | 2 | 3 | 1 | 1 | 2 | 3 | 2 | 3 | 2 | 2 | 1 | 1 | 2 | 3 | 1 | 1 | 3 | 1 | 2 | 1 | 2 | 1 | 2 |
| 3 | 1 | 1 | 1 | 3 | 2 | 2 | 1 | 2 | 2 | 1 | 3 | 2 | 1 | 3 | 2 | 2 | 1 | 1 | 1 | 3 | 1 | 2 | 3 |
| 2 | 1 | 3 | 1 | 1 | 2 | 2 | 3 | 2 | 3 | 2 | 2 | 2 | 3 | 1 | 2 | 1 | 1 | 3 | 2 | 3 | 1 | 2 | 1 |
| 2 | 3 | 1 | 2 | 2 | 2 | 1 | 3 | 1 | 2 | 2 | 3 | 1 | 3 | 1 | 3 | 2 | 2 | 1 | 1 | 1 | 2 | 3 | 1 |
| 1 | 2 | 2 | 1 | 2 | 2 | 3 | 1 | 3 | 2 | 2 | 2 | 3 | 1 | 1 | 2 | 3 | 2 | 2 | 3 | 1 | 2 | 1 | 3 |
| 1 | 2 | 1 | 3 | 2 | 1 | 3 | 2 | 2 | 1 | 2 | 3 | 2 | 2 | 2 | 3 | 1 | 2 | 2 | 2 | 1 | 3 | 2 | 3 |
| 1 | 2 | 1 | 3 | 1 | 1 | 3 | 1 | 1 | 3 | 1 | 1 | 2 | 1 | 1 | 1 | 3 | 2 | 2 | 1 | 3 | 1 | 3 | 1 |
| 3 | 1 | 2 | 3 | 2 | 2 | 3 | 1 | 1 | 1 | 3 | 2 | 1 | 1 | 2 | 3 | 1 | 1 | 2 | 2 | 2 | 3 | 2 | 1 |
| 3 | 1 | 3 | 1 | 2 | 2 | 3 | 1 | 2 | 1 | 3 | 2 | 1 | 3 | 1 | 1 | 1 | 2 | 3 | 1 | 2 | 1 | 1 | 1 |
| 2 | 3 | 1 | 3 | 1 | 3 | 1 | 1 | 2 | 1 | 1 | 1 | 3 | 2 | 1 | 2 | 3 | 1 | 1 | 2 | 2 | 2 | 3 | 1 |
| 2 | 1 | 2 | 1 | 1 | 1 | 3 | 1 | 2 | 3 | 1 | 2 | 3 | 2 | 3 | 1 | 1 | 2 | 2 | 1 | 3 | 2 | 1 | 3 |
| 2 | 2 | 1 | 2 | 3 | 2 | 1 | 1 | 3 | 1 | 1 | 2 | 3 | 2 | 2 | 2 | 3 | 1 | 3 | 1 | 3 | 1 | 1 | 1 |
| 1 | 3 | 2 | 1 | 1 | 1 | 2 | 3 | 1 | 2 | 3 | 1 | 1 | 2 | 3 | 1 | 2 | 1 | 2 | 3 | 1 | 2 | 3 | 1 |
| 3 | 1 | 1 | 1 | 2 | 2 | 2 | 3 | 2 | 3 | 2 | 2 | 1 | 1 | 1 | 3 | 2 | 2 | 3 | 1 | 1 | 2 | 3 | 1 |
| 3 | 1 | 2 | 3 | 1 | 1 | 2 | 3 | 1 | 2 | 2 | 3 | 2 | 3 | 2 | 2 | 2 | 1 | 1 | 3 | 2 | 1 | 2 | 1 |
| 3 | 1 | 1 | 1 | 2 | 1 | 1 | 3 | 2 | 3 | 1 | 3 | 1 | 3 | 2 | 2 | 1 | 1 | 2 | 3 | 1 | 1 | 1 | 2 |
| 2 | 3 | 2 | 2 | 3 | 1 | 1 | 1 | 2 | 1 | 3 | 2 | 2 | 1 | 2 | 2 | 1 | 3 | 2 | 2 | 2 | 3 | 2 | 3 |
| 2 | 2 | 2 | 3 | 1 | 3 | 1 | 3 | 2 | 1 | 2 | 1 | 2 | 2 | 3 | 1 | 2 | 1 | 2 | 3 | 1 | 3 | 1 | 1 |
| 1 | 2 | 2 | 3 | 2 | 3 | 2 | 3 | 2 | 1 | 1 | 1 | 3 | 2 | 1 | 1 | 3 | 1 | 2 | 2 | 2 | 1 | 1 | 3 |
| 2 | 1 | 2 | 1 | 3 | 2 | 2 | 2 | 3 | 1 | 1 | 3 | 2 | 3 | 2 | 3 | 1 | 2 | 3 | 2 | 1 | 2 | 2 | 2 |
| 3 | 2 | 3 | 1 | 1 | 3 | 2 | 2 | 1 | 2 | 1 | 3 | 2 | 3 | 2 | 1 | 2 | 1 | 1 | 1 | 3 | 1 | 1 | 2 |
| 3 | 2 | 1 | 2 | 3 | 2 | 2 | 3 | 1 | 1 | 2 | 1 | 3 | 2 | 1 | 1 | 1 | 2 | 1 | 3 | 1 | 2 | 2 | 3 |
| 2 | 2 | 1 | 3 | 1 | 1 | 1 | 3 | 2 | 3 | 2 | 3 | 1 | 2 | 2 | 2 | 3 | 2 | 3 | 2 | 1 | 2 | 2 | 2 |
| 2 | 2 | 2 | 1 | 3 | 2 | 1 | 1 | 2 | 1 | 2 | 3 | 2 | 1 | 1 | 3 | 1 | 3 | 1 | 2 | 3 | 2 | 3 | 1 |
| 1 | 3 | 2 | 1 | 2 | 3 | 2 | 1 | 2 | 1 | 3 | 1 | 2 | 3 | 1 | 2 | 3 | 2 | 2 | 2 | 3 | 2 | 2 | 2 |
| 1 | 2 | 2 | 2 | 1 | 1 | 3 | 2 | 1 | 1 | 1 | 3 | 2 | 3 | 2 | 1 | 3 | 1 | 3 | 1 | 2 | 1 | 1 | 3 |
| 1 | 2 | 2 | 2 | 3 | 2 | 3 | 2 | 2 | 3 | 1 | 1 | 2 | 2 | 3 | 2 | 1 | 1 | 1 | 3 | 2 | 3 | 1 | 1 |
| 1 | 2 | 3 | 2 | 2 | 1 | 2 | 2 | 1 | 3 | 1 | 2 | 2 | 3 | 2 | 3 | 1 | 2 | 3 | 1 | 1 | 2 | 3 | 1 |
| 2 | 1 | 3 | 2 | 1 | 3 | 2 | 1 | 3 | 1 | 1 | 2 | 1 | 2 | 3 | 1 | 1 | 1 | 2 | 2 | 1 | 3 | 1 | 3 |
| 2 | 2 | 2 | 1 | 1 | 2 | 3 | 1 | 3 | 1 | 1 | 3 | 1 | 3 | 2 | 2 | 1 | 3 | 1 | 3 | 2 | 1 | 2 | 1 |
| 1 | 1 | 1 | 3 | 2 | 2 | 2 | 1 | 3 | 2 | 1 | 3 | 1 | 3 | 2 | 3 | 2 | 1 | 2 | 3 | 2 | 1 | 1 | 1 |
| 1 | 2 | 1 | 2 | 1 | 2 | 3 | 1 | 2 | 1 | 3 | 2 | 1 | 3 | 1 | 3 | 2 | 1 | 3 | 1 | 2 | 2 | 1 | 3 |
| 2 | 3 | 1 | 3 | 1 | 1 | 3 | 2 | 2 | 1 | 1 | 2 | 2 | 3 | 2 | 1 | 2 | 1 | 3 | 1 | 2 | 2 | 3 | 1 |
| 2 | 1 | 2 | 1 | 3 | 1 | 3 | 1 | 2 | 3 | 2 | 2 | 1 | 2 | 1 | 2 | 3 | 1 | 1 | 3 | 2 | 2 | 3 | 2 |
| 1 | 1 | 1 | 2 | 2 | 2 | 3 | 2 | 2 | 1 | 1 | 3 | 2 | 2 | 3 | 2 | 2 | 3 | 2 | 2 | 3 | 2 | 2 | 3 |
| 2 | 2 | 3 | 2 | 2 | 3 | 1 | 1 | 3 | 1 | 2 | 3 | 1 | 1 | 1 | 3 | 2 | 1 | 3 | 1 | 1 | 2 | 2 | 1 |
| 1 | 1 | 3 | 1 | 3 | 1 | 2 | 1 | 1 | 3 | 2 | 1 | 3 | 2 | 3 | 2 | 2 | 2 | 1 | 2 | 3 | 2 | 2 | 2 |
| 1 | 1 | 2 | 1 | 1 | 3 | 1 | 1 | 3 | 1 | 1 | 3 | 2 | 3 | 1 | 1 | 1 | 3 | 1 | 2 | 2 | 3 | 1 | 2 |
| 2 | 1 | 1 | 3 | 2 | 2 | 1 | 1 | 1 | 3 | 2 | 2 | 3 | 1 | 2 | 3 | 1 | 2 | 2 | 3 | 1 | 2 | 1 | 3 |
| 1 | 2 | 1 | 2 | 1 | 1 | 3 | 1 | 2 | 1 | 1 | 3 | 1 | 3 | 2 | 3 | 2 | 1 | 1 | 3 | 2 | 3 | 1 | 2 |
| 3 | 2 | 2 | 1 | 1 | 1 | 2 | 3 | 2 | 2 | 3 | 2 | 2 | 3 | 2 | 2 | 2 | 1 | 1 | 3 | 2 | 3 | 1 | 2 |
| 3 | 1 | 3 | 2 | 2 | 1 | 1 | 3 | 2 | 2 | 1 | 2 | 2 | 1 | 3 | 2 | 2 | 1 | 1 | 3 | 1 | 1 | 3 | 2 |
| 2 | 1 | 2 | 2 | 1 | 3 | 1 | 3 | 2 | 2 | 2 | 3 | 1 | 3 | 1 | 1 | 2 | 1 | 1 | 3 | 2 | 1 | 3 | 2 |
| 2 | 1 | 1 | 2 | 3 | 2 | 2 | 3 | 2 | 2 | 1 | 2 | 3 | 2 | 3 | 2 | 2 | 1 | 3 | 1 | 2 | 3 | 2 | 2 |
| 3 | 1 | 1 | 1 | 3 | 2 | 2 | 3 | 1 | 2 | 1 | 3 | 1 | 1 | 2 | 3 | 2 | 1 | 1 | 2 | 3 | 2 | 2 | 2 |
| 2 | 3 | 1 | 2 | 1 | 3 | 1 | 2 | 3 | 1 | 1 | 2 | 2 | 3 | 1 | 2 | 2 | 3 | 1 | 2 | 2 | 1 | 3 | 2 |
| 1 | 2 | 3 | 1 | 2 | 1 | 3 | 1 | 3 | 2 | 1 | 1 | 1 | 3 | 1 | 1 | 2 | 1 | 1 | 3 | 2 | 2 | 3 | 2 |
| 1 | 3 | 2 | 1 | 1 | 3 | 2 | 3 | 2 | 2 | 1 | 3 | 1 | 2 | 1 | 3 | 2 | 1 | 2 | 2 | 3 | 1 | 1 | 2 |
| 1 | 2 | 3 | 2 | 1 | 3 | 1 | 2 | 2 | 1 | 1 | 1 | 3 | 2 | 1 | 3 | 2 | 3 | 2 | 1 | 2 | 3 | 2 | 2 |
| 2 | 2 | 1 | 2 | 2 | 3 | 1 | 2 | 1 | 1 | 2 | 3 | 1 | 3 | 1 | 3 | 1 | 3 | 2 | 2 | 1 | 1 | 1 | 3 |
| 1 | 2 | 2 | 2 | 3 | 2 | 2 | 1 | 2 | 3 | 1 | 2 | 1 | 1 | 1 | 2 | 3 | 2 | 3 | 2 | 1 | 3 | 2 | 3 |
| 2 | 2 | 3 | 1 | 1 | 3 | 1 | 1 | 1 | 2 | 1 | 1 | 3 | 1 | 3 | 2 | 1 | 1 | 2 | 1 | 1 | 3 | 1 | 3 |
| 2 | 3 | 2 | 3 | 2 | 1 | 1 | 2 | 1 | 1 | 3 | 2 | 1 | 3 | 2 | 1 | 1 | 3 | 1 | 2 | 2 | 1 | 3 | 1 |
| 1 | 2 | 3 | 1 | 1 | 1 | 3 | 2 | 2 | 1 | 3 | 1 | 3 | 2 | 2 | 2 | 1 | 2 | 3 | 1 | 2 | 1 | 1 | 3 |
| 1 | 2 | 2 | 1 | 3 | 2 | 2 | 1 | 1 | 3 | 1 | 3 | 1 | 3 | 2 | 2 | 2 | 3 | 2 | 1 | 3 | 1 | 2 | 2 |
| 2 | 3 | 2 | 1 | 3 | 2 | 1 | 2 | 2 | 3 | 2 | 1 | 2 | 3 | 1 | 2 | 2 | 1 | 1 | 1 | 3 | 2 | 3 | 2 |
| 1 | 3 | 2 | 2 | 3 | 1 | 2 | 1 | 1 | 1 | 3 | 1 | 1 | 3 | 1 | 1 | 3 | 1 | 3 | 2 | 1 | 2 | 1 | 2 |
| 3 | 2 | 1 | 1 | 2 | 3 | 1 | 3 | 1 | 2 | 1 | 1 | 1 | 3 | 1 | 3 | 1 | 3 | 1 | 2 | 1 | 1 | 2 | 2 |
| 2 | 3 | 2 | 3 | 2 | 2 | 3 | 1 | 1 | 3 | 1 | 2 | 1 | 1 | 1 | 3 | 2 | 2 | 2 | 1 | 2 | 3 | 1 | 2 |
| 1 | 1 | 3 | 1 | 1 | 3 | 1 | 3 | 2 | 1 | 3 | 2 | 2 | 1 | 3 | 1 | 1 | 2 | 2 | 3 | 1 | 2 | 2 | 1 |
| 3 | 1 | 1 | 2 | 3 | 1 | 1 | 3 | 1 | 2 | 3 | 1 | 1 | 3 | 2 | 2 | 2 | 3 | 2 | 2 | 1 | 1 | 2 | 1 |
| 1 | 1 | 1 | 2 | 2 | 3 | 2 | 2 | 3 | 1 | 3 | 1 | 2 | 1 | 1 | 3 | 1 | 2 | 1 | 3 | 2 | 3 | 1 | 2 |
| 2 | 3 | 1 | 2 | 2 | 3 | 2 | 2 | 2 | 1 | 1 | 2 | 3 | 1 | 2 | 3 | 2 | 3 | 2 | 3 | 1 | 2 | 2 | 1 |
| 1 | 2 | 3 | 1 | 1 | 3 | 2 | 1 | 2 | 2 | 3 | 2 | 2 | 3 | 1 | 3 | 2 | 3 | 1 | 2 | 2 | 2 | 1 | 1 |
| 3 | 2 | 3 | 2 | 1 | 1 | 1 | 2 | 3 | 2 | 2 | 2 | 3 | 1 | 3 | 1 | 2 | 3 | 2 | 1 | 2 | 1 | 2 | 2 |
| 1 | 1 | 2 | 2 | 3 | 1 | 2 | 3 | 1 | 3 | 2 | 2 | 2 | 1 | 1 | 1 | 3 | 1 | 3 | 2 | 2 | 3 | 1 | 2 |
| 2 | 2 | 2 | 3 | 2 | 3 | 2 | 1 | 1 | 2 | 1 | 2 | 3 | 1 | 2 | 2 | 3 | 1 | 3 | 1 | 3 | 2 | 2 | 2 |
| 3 | 2 | 1 | 3 | 2 | 1 | 3 | 1 | 2 | 3 | 1 | 2 | 2 | 1 | 1 | 3 | 1 | 1 | 3 | 1 | 2 | 1 | 1 | 1 |
| 2 | 2 | 2 | 1 | 1 | 2 | 3 | 2 | 3 | 1 | 1 | 1 | 2 | 2 | 2 | 3 | 2 | 2 | 3 | 2 | 3 | 1 | 3 | 2 |
| 3 | 2 | 1 | 1 | 1 | 3 | 1 | 1 | 2 | 2 | 1 | 3 | 1 | 2 | 1 | 1 | 1 | 3 | 1 | 3 | 2 | 3 | 1 | 2 |
| 1 | 1 | 2 | 1 | 3 | 2 | 2 | 1 | 1 | 3 | 2 | 2 | 2 | 1 | 1 | 3 | 1 | 3 | 2 | 2 | 3 | 2 | 3 | 2 |
| 3 | 2 | 3 | 2 | 3 | 1 | 2 | 3 | 2 | 2 | 2 | 1 | 2 | 1 | 3 | 1 | 2 | 2 | 2 | 3 | 2 | 2 | 1 | 2 |
| 3 | 2 | 1 | 2 | 1 | 3 | 2 | 3 | 2 | 3 | 1 | 2 | 2 | 1 | 3 | 1 | 2 | 2 | 2 | 3 | 2 | 1 | 1 | 1 |
| 3 | 2 | 2 | 3 | 2 | 1 | 1 | 3 | 1 | 1 | 1 | 3 | 1 | 2 | 1 | 2 | 3 | 2 | 1 | 1 | 3 | 1 | 1 | 1 |
| 1 | 2 | 1 | 2 | 2 | 1 | 3 | 1 | 2 | 2 | 3 | 2 | 1 | 1 | 1 | 3 | 1 | 3 | 1 | 3 | 2 | 3 | 1 | 1 |
| 3 | 1 | 3 | 2 | 3 | 1 | 2 | 1 | 2 | 2 | 3 | 1 | 1 | 1 | 2 | 2 | 1 | 3 | 1 | 2 | 2 | 3 | 2 | 1 |
| 2 | 1 | 1 | 3 | 1 | 1 | 3 | 2 | 2 | 1 | 1 | 1 | 3 | 1 | 1 | 3 | 1 | 3 | 1 | 3 | 2 | 2 | 2 | 1 |
| 3 | 1 | 2 | 3 | 2 | 2 | 1 | 3 | 1 | 2 | 1 | 1 | 1 | 3 | 2 | 2 | 2 | 1 | 1 | 3 | 2 | 1 | 3 | 2 |
| 3 | 2 | 3 | 1 | 2 | 2 | 3 | 2 | 1 | 2 | 3 | 1 | 3 | 1 | 1 | 1 | 2 | 3 | 2 | 2 | 1 | 1 | 1 | 2 |
| 2 | 3 | 1 | 2 | 2 | 1 | 2 | 2 | 3 | 2 | 1 | 1 | 3 | 1 | 1 | 1 | 3 | 1 | 2 | 2 | 3 | 1 | 3 | 1 |
| 1 | 1 | 3 | 1 | 1 | 2 | 2 | 3 | 2 | 3 | 2 | 1 | 1 | 3 | 2 | 2 | 2 | 1 | 2 | 3 | 1 | 3 | 2 | 1 |
| 2 | 2 | 3 | 2 | 1 | 2 | 2 | 2 | 1 | 3 | 1 | 1 | 3 | 1 | 2 | 2 | 2 | 3 | 2 | 1 | 3 | 1 | 2 | 3 |
| 2 | 1 | 2 | 1 | 2 | 3 | 2 | 2 | 2 | 3 | 2 | 3 | 2 | 1 | 1 | 3 | 1 | 1 | 3 | 1 | 1 | 1 | 2 | 3 |
| 3 | 1 | 2 | 1 | 1 | 2 | 3 | 2 | 3 | 2 | 3 | 1 | 1 | 2 | 2 | 2 | 3 | 2 | 3 | 1 | 1 | 2 | 1 | 1 |
| 2 | 2 | 1 | 3 | 1 | 1 | 1 | 2 | 3 | 2 | 3 | 1 | 3 | 1 | 2 | 2 | 2 | 1 | 1 | 3 | 1 | 3 | 2 | 2 |
| 1 | 3 | 2 | 3 | 2 | 1 | 3 | 1 | 1 | 2 | 2 | 2 | 3 | 2 | 1 | 2 | 2 | 2 | 1 | 3 | 2 | 2 | 3 | 2 |
| 2 | 1 | 3 | 2 | 2 | 1 | 1 | 3 | 1 | 2 | 1 | 3 | 1 | 3 | 2 | 1 | 1 | 1 | 2 | 3 | 1 | 2 | 1 | 3 |
| 3 | 1 | 1 | 3 | 2 | 3 | 1 | 2 | 1 | 2 | 2 | 3 | 2 | 1 | 1 | 1 | 2 | 2 | 3 | 1 | 2 | 1 | 1 | 3 |
| 3 | 2 | 1 | 1 | 2 | 2 | 3 | 2 | 3 | 2 | 2 | 1 | 3 | 1 | 2 | 2 | 2 | 1 | 1 | 3 | 1 | 1 | 3 | 2 |
| 2 | 3 | 1 | 2 | 1 | 2 | 2 | 2 | 3 | 2 | 3 | 1 | 1 | 2 | 2 | 3 | 1 | 2 | 1 | 3 | 2 | 1 | 2 | 3 |
| 1 | 1 | 3 | 2 | 1 | 1 | 1 | 3 | 1 | 3 | 1 | 2 | 1 | 2 | 1 | 3 | 2 | 2 | 1 | 1 | 3 | 2 | 2 | 3 |
| 1 | 2 | 2 | 1 | 3 | 2 | 2 | 1 | 1 | 3 | 2 | 2 | 1 | 2 | 2 | 2 | 3 | 2 | 3 | 1 | 3 | 2 | 3 | 1 |
| 1 | 3 | 1 | 2 | 3 | 1 | 1 | 3 | 2 | 1 | 3 | 2 | 2 | 2 | 1 | 2 | 3 | 1 | 1 | 2 | 2 | 1 | 3 | 1 |
| 2 | 3 | 1 | 3 | 2 | 2 | 1 | 3 | 2 | 2 | 1 | 1 | 3 | 2 | 3 | 1 | 2 | 1 | 3 | 2 | 2 | 1 | 1 | 1 |
| 2 | 2 | 1 | 2 | 2 | 3 | 2 | 1 | 3 | 1 | 2 | 2 | 2 | 1 | 3 | 1 | 3 | 1 | 1 | 3 | 1 | 2 | 3 | 1 |
| 2 | 1 | 2 | 2 | 2 | 3 | 2 | 3 | 2 | 2 | 2 | 3 | 2 | 2 | 3 | 1 | 2 | 2 | 1 | 3 | 1 | 2 | 1 | 3 |
| 3 | 2 | 1 | 2 | 1 | 1 | 2 | 3 | 2 | 3 | 2 | 3 | 2 | 3 | 1 | 1 | 1 | 3 | 2 | 2 | 1 | 2 | 1 | 1 |
| 2 | 1 | 2 | 1 | 2 | 3 | 2 | 2 | 3 | 1 | 3 | 2 | 1 | 2 | 1 | 1 | 1 | 3 | 1 | 3 | 1 | 3 | 1 | 1 |
| 2 | 2 | 1 | 3 | 2 | 2 | 1 | 3 | 2 | 2 | 2 | 1 | 1 | 1 | 3 | 1 | 2 | 2 | 3 | 2 | 3 | 1 | 3 | 2 |
| 2 | 2 | 2 | 1 | 3 | 1 | 1 | 2 | 1 | 1 | 3 | 2 | 3 | 1 | 2 | 3 | 2 | 3 | 1 | 2 | 3 | 1 | 1 | 1 |
| 1 | 3 | 1 | 3 | 2 | 1 | 1 | 2 | 3 | 2 | 3 | 2 | 1 | 1 | 1 | 2 | 1 | 3 | 2 | 2 | 1 | 3 | 1 | 2 |
| 2 | 3 | 2 | 3 | 1 | 2 | 1 | 1 | 1 | 3 | 1 | 3 | 1 | 1 | 1 | 2 | 2 | 1 | 3 | 2 | 2 | 3 | 2 | 2 |
| 3 | 1 | 1 | 2 | 2 | 2 | 1 | 3 | 2 | 3 | 1 | 1 | 2 | 3 | 2 | 2 | 2 | 3 | 1 | 3 | 1 | 2 | 2 | 1 |
| 2 | 3 | 2 | 3 | 1 | 2 | 3 | 2 | 3 | 2 | 1 | 1 | 3 | 2 | 1 | 2 | 1 | 2 | 3 | 1 | 1 | 1 | 2 | 2 |
| 2 | 2 | 3 | 2 | 3 | 1 | 1 | 2 | 3 | 1 | 2 | 2 | 1 | 1 | 2 | 3 | 1 | 1 | 2 | 1 | 3 | 1 | 1 | 3 |
| 1 | 1 | 2 | 3 | 2 | 2 | 3 | 2 | 2 | 2 | 1 | 3 | 1 | 2 | 2 | 3 | 1 | 3 | 1 | 1 | 1 | 3 | 2 | 2 |
| 1 | 3 | 1 | 2 | 2 | 3 | 2 | 3 | 2 | 2 | 1 | 3 | 2 | 1 | 2 | 2 | 1 | 3 | 2 | 1 | 2 | 1 | 1 | 3 |
| 2 | 2 | 3 | 1 | 2 | 3 | 2 | 1 | 2 | 2 | 1 | 3 | 1 | 1 | 1 | 3 | 2 | 2 | 2 | 1 | 2 | 3 | 2 | 3 |
| 2 | 1 | 2 | 3 | 1 | 2 | 2 | 3 | 2 | 3 | 2 | 3 | 2 | 2 | 1 | 3 | 1 | 3 | 1 | 1 | 2 | 2 | 2 | 1 |
| 2 | 1 | 3 | 2 | 3 | 2 | 1 | 3 | 1 | 2 | 1 | 2 | 2 | 2 | 3 | 1 | 2 | 1 | 3 | 2 | 2 | 1 | 2 | 3 |
| 1 | 3 | 2 | 2 | 2 | 1 | 1 | 2 | 3 | 2 | 3 | 2 | 2 | 2 | 1 | 3 | 2 | 2 | 3 | 2 | 2 | 1 | 2 | 3 |
| 2 | 3 | 2 | 3 | 2 | 1 | 1 | 1 | 3 | 2 | 1 | 3 | 1 | 1 | 1 | 3 | 2 | 1 | 1 | 1 | 3 | 1 | 2 | 2 |
| 3 | 2 | 2 | 1 | 2 | 3 | 1 | 2 | 1 | 2 | 1 | 3 | 2 | 3 | 1 | 3 | 2 | 2 | 3 | 2 | 2 | 1 | 2 | 2 |
| 2 | 2 | 2 | 3 | 1 | 2 | 2 | 3 | 1 | 1 | 2 | 3 | 2 | 2 | 1 | 1 | 2 | 1 | 3 | 2 | 3 | 2 | 3 | 2 |
| 1 | 3 | 1 | 3 | 2 | 1 | 2 | 2 | 1 | 3 | 2 | 1 | 3 | 2 | 2 | 1 | 2 | 2 | 3 | 2 | 1 | 1 | 3 | 2 |
| 2 | 1 | 1 | 3 | 2 | 1 | 3 | 1 | 1 | 1 | 3 | 1 | 1 | 3 | 1 | 1 | 3 | 1 | 2 | 1 | 2 | 2 | 2 | 3 |
| 1 | 3 | 1 | 1 | 1 | 3 | 1 | 3 | 1 | 1 | 2 | 2 | 1 | 2 | 3 | 2 | 1 | 1 | 2 | 3 | 1 | 1 | 1 | 3 |
| 2 | 2 | 1 | 3 | 1 | 2 | 2 | 2 | 3 | 2 | 2 | 1 | 3 | 2 | 3 | 2 | 3 | 1 | 2 | 2 | 2 | 1 | 1 | 3 |
| 3 | 1 | 2 | 3 | 1 | 2 | 2 | 1 | 1 | 3 | 1 | 2 | 1 | 2 | 1 | 3 | 1 | 3 | 1 | 2 | 1 | 3 | 2 | 2 |
| 1 | 2 | 1 | 2 | 2 | 2 | 3 | 1 | 3 | 2 | 3 | 1 | 2 | 2 | 1 | 1 | 3 | 1 | 3 | 2 | 1 | 1 | 2 | 3 |
| 2 | 3 | 2 | 1 | 2 | 2 | 3 | 2 | 3 | 1 | 3 | 2 | 2 | 1 | 1 | 3 | 2 | 1 | 2 | 1 | 1 | 3 | 2 | 2 |
| 1 | 1 | 2 | 2 | 2 | 1 | 3 | 2 | 1 | 3 | 1 | 1 | 1 | 3 | 2 | 3 | 2 | 2 | 3 | 2 | 3 | 2 | 2 | 2 |
| 3 | 2 | 2 | 1 | 3 | 1 | 1 | 3 | 1 | 2 | 2 | 1 | 1 | 3 | 2 | 2 | 3 | 1 | 1 | 2 | 1 | 1 | 2 | 3 |
| 2 | 1 | 1 | 1 | 3 | 2 | 1 | 2 | 3 | 2 | 3 | 1 | 3 | 1 | 2 | 3 | 1 | 2 | 2 | 2 | 1 | 2 | 3 | 2 |
| 2 | 3 | 1 | 1 | 1 | 2 | 3 | 1 | 2 | 2 | 1 | 1 | 1 | 3 | 1 | 2 | 3 | 1 | 1 | 3 | 1 | 2 | 3 | 1 |
| 2 | 2 | 1 | 2 | 2 | 1 | 3 | 1 | 2 | 3 | 2 | 2 | 3 | 1 | 3 | 2 | 3 | 2 | 2 | 2 | 3 | 2 | 2 | 2 |
| 2 | 1 | 3 | 2 | 3 | 2 | 2 | 2 | 1 | 1 | 1 | 3 | 1 | 3 | 2 | 1 | 3 | 2 | 1 | 2 | 1 | 2 | 3 | 1 |
| 1 | 3 | 2 | 2 | 1 | 2 | 1 | 1 | 3 | 2 | 1 | 1 | 1 | 2 | 3 | 1 | 2 | 3 | 2 | 2 | 3 | 1 | 2 | 3 |
| 2 | 2 | 1 | 1 | 3 | 1 | 3 | 1 | 3 | 1 | 1 | 1 | 2 | 1 | 1 | 3 | 2 | 3 | 2 | 1 | 2 | 2 | 3 | 2 |
| 3 | 1 | 2 | 1 | 2 | 2 | 3 | 1 | 1 | 1 | 2 | 3 | 2 | 3 | 2 | 1 | 1 | 1 | 2 | 3 | 1 | 2 | 3 | 1 |
| 1 | 2 | 3 | 1 | 2 | 3 | 1 | 1 | 2 | 2 | 1 | 1 | 3 | 1 | 1 | 1 | 3 | 1 | 1 | 1 | 3 | 1 | 3 | 1 |
| 3 | 1 | 1 | 2 | 1 | 3 | 2 | 2 | 2 | 3 | 1 | 2 | 2 | 2 | 3 | 2 | 3 | 2 | 2 | 2 | 3 | 2 | 2 | 1 |
| 1 | 3 | 2 | 2 | 3 | 2 | 2 | 2 | 1 | 3 | 1 | 2 | 2 | 2 | 3 | 1 | 2 | 2 | 2 | 3 | 2 | 1 | 1 | 3 |
| 3 | 2 | 1 | 2 | 3 | 1 | 3 | 1 | 2 | 2 | 2 | 3 | 1 | 2 | 1 | 2 | 1 | 1 | 3 | 1 | 2 | 2 | 1 | 3 |
| 2 | 2 | 2 | 1 | 2 | 1 | 3 | 2 | 3 | 1 | 3 | 2 | 1 | 2 | 1 | 3 | 2 | 3 | 1 | 2 | 3 | 1 | 2 | 2 |
| 2 | 1 | 2 | 1 | 2 | 3 | 2 | 3 | 1 | 1 | 3 | 1 | 2 | 1 | 2 | 1 | 1 | 3 | 2 | 3 | 2 | 2 | 2 | 3 |
| 1 | 2 | 2 | 3 | 1 | 2 | 1 | 3 | 1 | 2 | 3 | 1 | 2 | 1 | 3 | 2 | 1 | 1 | 2 | 2 | 3 | 1 | 3 | 2 |
| 2 | 3 | 1 | 2 | 1 | 3 | 1 | 2 | 3 | 2 | 3 | 1 | 1 | 3 | 1 | 1 | 2 | 2 | 2 | 3 | 1 | 2 | 2 | 2 |
| 3 | 1 | 1 | 3 | 1 | 2 | 1 | 2 | 2 | 3 | 1 | 1 | 1 | 3 | 1 | 1 | 2 | 2 | 2 | 3 | 1 | 2 | 3 | 2 |
| 3 | 1 | 2 | 3 | 2 | 2 | 2 | 1 | 3 | 2 | 3 | 2 | 1 | 3 | 1 | 2 | 1 | 2 | 1 | 3 | 1 | 2 | 2 | 2 |
| 3 | 1 | 1 | 2 | 1 | 2 | 2 | 3 | 1 | 3 | 2 | 2 | 1 | 2 | 1 | 1 | 3 | 2 | 1 | 3 | 2 | 1 | 3 | 2 |
| 1 | 3 | 2 | 3 | 1 | 3 | 2 | 1 | 1 | 3 | 2 | 1 | 1 | 2 | 1 | 3 | 1 | 1 | 1 | 3 | 1 | 2 | 2 | 2 |
| 3 | 2 | 1 | 3 | 1 | 1 | 2 | 1 | 1 | 3 | 2 | 1 | 1 | 2 | 2 | 2 | 3 | 2 | 3 | 1 | 3 | 1 | 2 | 1 |
| 3 | 1 | 3 | 2 | 2 | 1 | 2 | 2 | 2 | 3 | 1 | 3 | 1 | 2 | 2 | 2 | 1 | 3 | 1 | 2 | 3 | 2 | 2 | 2 |
| 3 | 1 | 1 | 1 | 2 | 3 | 1 | 2 | 3 | 1 | 2 | 2 | 3 | 1 | 1 | 2 | 2 | 2 | 1 | 3 | 1 | 3 | 1 | 2 |
| 1 | 1 | 1 | 2 | 1 | 3 | 2 | 3 | 2 | 3 | 1 | 3 | 1 | 1 | 2 | 1 | 3 | 2 | 2 | 1 | 1 | 3 | 2 | 1 |
| 1 | 2 | 3 | 2 | 3 | 2 | 2 | 1 | 1 | 3 | 2 | 2 | 3 | 2 | 1 | 3 | 1 | 1 | 3 | 1 | 1 | 2 | 1 | 1 |
| 1 | 2 | 1 | 1 | 2 | 3 | 1 | 3 | 2 | 2 | 1 | 1 | 2 | 1 | 3 | 2 | 3 | 2 | 1 | 1 | 3 | 1 | 1 | 3 |
| 1 | 2 | 1 | 1 | 3 | 1 | 3 | 1 | 2 | 3 | 2 | 2 | 2 | 1 | 1 | 3 | 2 | 2 | 1 | 3 | 1 | 1 | 1 | 3 |
| 2 | 3 | 2 | 2 | 1 | 3 | 2 | 3 | 2 | 2 | 1 | 3 | 1 | 1 | 1 | 2 | 1 | 2 | 3 | 1 | 1 | 1 | 3 | 1 |
| 2 | 2 | 2 | 1 | 3 | 1 | 1 | 3 | 1 | 2 | 2 | 3 | 2 | 2 | 1 | 3 | 1 | 2 | 1 | 1 | 3 | 2 | 2 | 3 |
| 3 | 2 | 3 | 2 | 1 | 1 | 2 | 3 | 2 | 1 | 2 | 1 | 1 | 3 | 1 | 2 | 1 | 3 | 2 | 2 | 1 | 1 | 3 | 2 |
| 2 | 1 | 2 | 2 | 1 | 3 | 1 | 3 | 1 | 3 | 1 | 1 | 1 | 2 | 2 | 3 | 2 | 1 | 3 | 1 | 3 | 1 | 2 | 2 |
| 2 | 1 | 3 | 2 | 3 | 1 | 3 | 1 | 2 | 1 | 1 | 1 | 3 | 2 | 1 | 1 | 1 | 3 | 2 | 2 | 2 | 1 | 2 | 3 |
| 2 | 2 | 3 | 2 | 3 | 1 | 1 | 1 | 3 | 2 | 2 | 1 | 1 | 3 | 2 | 1 | 1 | 3 | 2 | 2 | 1 | 3 | 2 | 2 |
| 1 | 1 | 1 | 3 | 2 | 3 | 2 | 1 | 1 | 3 | 2 | 2 | 3 | 1 | 1 | 3 | 1 | 1 | 2 | 1 | 2 | 2 | 3 | 1 |
| 3 | 1 | 1 | 2 | 1 | 3 | 1 | 3 | 2 | 3 | 2 | 2 | 1 | 2 | 2 | 2 | 3 | 1 | 1 | 1 | 2 | 1 | 3 | 1 |
| 2 | 1 | 2 | 1 | 1 | 3 | 1 | 3 | 1 | 3 | 1 | 3 | 1 | 2 | 1 | 1 | 3 | 2 | 1 | 1 | 2 | 1 | 1 | 3 |
| 2 | 3 | 1 | 3 | 2 | 3 | 1 | 1 | 1 | 2 | 2 | 3 | 1 | 2 | 1 | 3 | 1 | 3 | 2 | 1 | 1 | 1 | 2 | 2 |
| 3 | 1 | 2 | 3 | 1 | 1 | 2 | 1 | 1 | 3 | 2 | 2 | 2 | 1 | 1 | 3 | 2 | 3 | 1 | 3 | 1 | 1 | 1 | 2 |
| 3 | 2 | 3 | 2 | 3 | 1 | 2 | 1 | 2 | 3 | 2 | 2 | 2 | 1 | 2 | 2 | 3 | 1 | 2 | 2 | 1 | 1 | 3 | 2 |
| 2 | 1 | 1 | 1 | 3 | 2 | 3 | 1 | 3 | 2 | 3 | 2 | 1 | 1 | 1 | 2 | 3 | 1 | 2 | 1 | 1 | 2 | 3 | 1 |
| 3 | 2 | 1 | 3 | 1 | 3 | 2 | 2 | 2 | 3 | 1 | 2 | 2 | 2 | 3 | 1 | 1 | 1 | 3 | 1 | 1 | 2 | 1 | 2 |
| 3 | 1 | 1 | 2 | 1 | 2 | 2 | 3 | 2 | 2 | 1 | 2 | 3 | 2 | 2 | 2 | 3 | 2 | 2 | 1 | 2 | 3 | 1 | 3 |
| 3 | 2 | 3 | 2 | 1 | 1 | 2 | 1 | 1 | 3 | 1 | 2 | 3 | 2 | 1 | 2 | 2 | 3 | 2 | 2 | 3 | 2 | 2 | 2 |
| 2 | 1 | 1 | 1 | 2 | 2 | 3 | 1 | 2 | 2 | 3 | 2 | 3 | 1 | 3 | 2 | 2 | 3 | 1 | 1 | 3 | 1 | 1 | 2 |
| 2 | 3 | 1 | 3 | 1 | 2 | 1 | 3 | 2 | 2 | 1 | 2 | 1 | 3 | 2 | 2 | 1 | 1 | 3 | 2 | 2 | 2 | 1 | 3 |
| 1 | 3 | 2 | 2 | 2 | 3 | 2 | 2 | 1 | 1 | 3 | 1 | 2 | 2 | 1 | 2 | 3 | 2 | 1 | 3 | 1 | 1 | 1 | 3 |
| 3 | 1 | 1 | 2 | 3 | 2 | 3 | 2 | 1 | 3 | 1 | 1 | 2 | 1 | 1 | 3 | 1 | 3 | 1 | 2 | 2 | 1 | 1 | 1 |
| 3 | 2 | 1 | 2 | 2 | 1 | 2 | 3 | 1 | 1 | 1 | 3 | 1 | 1 | 3 | 2 | 2 | 3 | 2 | 2 | 3 | 2 | 2 | 2 |
| 3 | 2 | 3 | 2 | 2 | 1 | 2 | 1 | 3 | 1 | 1 | 3 | 2 | 2 | 1 | 1 | 1 | 2 | 3 | 2 | 2 | 1 | 1 | 3 |
| 2 | 2 | 1 | 1 | 3 | 1 | 3 | 2 | 1 | 3 | 2 | 3 | 1 | 1 | 2 | 1 | 2 | 3 | 1 | 2 | 1 | 3 | 2 | 1 |
| 1 | 1 | 2 | 3 | 2 | 2 | 1 | 2 | 1 | 1 | 3 | 1 | 2 | 3 | 1 | 3 | 1 | 3 | 2 | 2 | 2 | 1 | 3 | 2 |
| 1 | 2 | 1 | 2 | 1 | 1 | 3 | 1 | 2 | 2 | 2 | 3 | 1 | 2 | 3 | 2 | 1 | 3 | 2 | 3 | 2 | 1 | 3 | 2 |
| 2 | 1 | 2 | 3 | 2 | 2 | 2 | 3 | 2 | 2 | 3 | 2 | 2 | 3 | 2 | 2 | 1 | 1 | 3 | 2 | 2 | 2 | 3 | 1 |
| 3 | 1 | 2 | 1 | 3 | 2 | 2 | 2 | 1 | 3 | 2 | 1 | 2 | 1 | 3 | 1 | 1 | 3 | 1 | 2 | 1 | 1 | 1 | 3 |
| 3 | 2 | 2 | 3 | 1 | 1 | 2 | 1 | 2 | 1 | 3 | 1 | 3 | 1 | 2 | 1 | 3 | 2 | 1 | 1 | 1 | 2 | 1 | 3 |
| 1 | 3 | 1 | 3 | 1 | 1 | 3 | 1 | 2 | 2 | 2 | 1 | 3 | 2 | 1 | 1 | 3 | 1 | 1 | 2 | 3 | 1 | 2 | 1 |
| 2 | 3 | 1 | 1 | 2 | 3 | 1 | 3 | 1 | 1 | 1 | 3 | 1 | 2 | 1 | 2 | 2 | 3 | 1 | 3 | 2 | 1 | 2 | 2 |
| 2 | 3 | 1 | 1 | 3 | 1 | 2 | 2 | 1 | 2 | 1 | 3 | 2 | 1 | 3 | 2 | 2 | 3 | 2 | 1 | 2 | 1 | 3 | 1 |
| 3 | 1 | 2 | 2 | 1 | 3 | 2 | 1 | 3 | 2 | 1 | 2 | 2 | 3 | 1 | 1 | 3 | 1 | 2 | 2 | 1 | 2 | 3 | 2 |
| 2 | 3 | 1 | 1 | 1 | 2 | 3 | 2 | 3 | 2 | 1 | 2 | 2 | 2 | 3 | 2 | 1 | 2 | 3 | 2 | 2 | 2 | 1 | 3 |
| 1 | 2 | 2 | 1 | 1 | 1 | 3 | 2 | 2 | 3 | 1 | 2 | 1 | 2 | 3 | 1 | 1 | 1 | 3 | 1 | 1 | 3 | 2 | 3 |
| 1 | 1 | 2 | 3 | 2 | 1 | 3 | 1 | 3 | 1 | 2 | 2 | 3 | 2 | 1 | 3 | 2 | 3 | 1 | 1 | 2 | 1 | 2 | 2 |
| 2 | 2 | 1 | 2 | 2 | 2 | 3 | 2 | 2 | 3 | 1 | 3 | 2 | 3 | 2 | 1 | 1 | 1 | 2 | 3 | 2 | 3 | 1 | 2 |
| 1 | 2 | 3 | 2 | 1 | 1 | 2 | 2 | 3 | 2 | 3 | 1 | 1 | 2 | 1 | 1 | 2 | 3 | 2 | 1 | 2 | 3 | 2 | 3 |
| 3 | 1 | 2 | 2 | 2 | 3 | 2 | 1 | 2 | 1 | 3 | 1 | 3 | 1 | 2 | 2 | 1 | 3 | 2 | 1 | 1 | 3 | 2 | 1 |
| 1 | 1 | 2 | 1 | 2 | 2 | 3 | 2 | 2 | 3 | 2 | 2 | 2 | 1 | 3 | 1 | 3 | 1 | 1 | 1 | 3 | 1 | 1 | 3 |
| 1 | 2 | 3 | 1 | 2 | 3 | 1 | 2 | 3 | 2 | 1 | 2 | 2 | 2 | 3 | 2 | 1 | 1 | 1 | 3 | 1 | 3 | 2 | 1 |
| 1 | 1 | 2 | 3 | 2 | 1 | 2 | 2 | 2 | 3 | 2 | 3 | 2 | 3 | 1 | 2 | 2 | 3 | 2 | 3 | 2 | 1 | 1 | 1 |
| 1 | 3 | 2 | 3 | 2 | 2 | 1 | 2 | 3 | 1 | 1 | 3 | 1 | 1 | 2 | 1 | 3 | 2 | 1 | 1 | 3 | 1 | 1 | 2 |
| 3 | 2 | 2 | 1 | 2 | 3 | 2 | 1 | 3 | 1 | 3 | 1 | 2 | 3 | 1 | 1 | 1 | 3 | 1 | 1 | 1 | 2 | 2 | 1 |
| 3 | 2 | 2 | 2 | 3 | 2 | 1 | 2 | 2 | 1 | 3 | 1 | 2 | 1 | 1 | 1 | 2 | 3 | 1 | 3 | 2 | 2 | 3 | 2 |
| 2 | 3 | 1 | 2 | 2 | 2 | 1 | 2 | 3 | 1 | 3 | 1 | 2 | 2 | 1 | 1 | 3 | 1 | 3 | 1 | 1 | 1 | 3 | 1 |
| 2 | 2 | 2 | 3 | 2 | 3 | 2 | 3 | 2 | 2 | 1 | 2 | 2 | 3 | 2 | 1 | 1 | 2 | 2 | 3 | 1 | 3 | 1 | 2 |
| 3 | 1 | 2 | 3 | 2 | 3 | 2 | 3 | 1 | 2 | 1 | 2 | 3 | 1 | 2 | 2 | 1 | 1 | 1 | 3 | 1 | 1 | 1 | 2 |
| 1 | 3 | 1 | 2 | 2 | 1 | 2 | 1 | 3 | 1 | 2 | 2 | 2 | 3 | 2 | 1 | 3 | 1 | 3 | 1 | 1 | 1 | 3 | 2 |
| 3 | 1 | 1 | 3 | 1 | 3 | 2 | 1 | 2 | 3 | 2 | 1 | 1 | 2 | 1 | 3 | 2 | 1 | 2 | 2 | 3 | 2 | 1 | 2 |
| 2 | 2 | 2 | 3 | 2 | 1 | 1 | 2 | 3 | 2 | 2 | 3 | 2 | 2 | 3 | 1 | 3 | 2 | 2 | 2 | 1 | 1 | 3 | 1 |
| 1 | 3 | 2 | 1 | 1 | 1 | 2 | 1 | 3 | 2 | 1 | 3 | 2 | 1 | 2 | 3 | 1 | 1 | 2 | 1 | 1 | 3 | 1 | 3 |
| 3 | 1 | 1 | 2 | 3 | 2 | 2 | 3 | 1 | 1 | 2 | 2 | 3 | 1 | 1 | 1 | 2 | 1 | 2 | 3 | 1 | 3 | 2 | 2 |
| 1 | 3 | 2 | 1 | 3 | 2 | 2 | 1 | 1 | 2 | 2 | 3 | 1 | 2 | 1 | 3 | 2 | 1 | 1 | 3 | 2 | 2 | 2 | 3 |
| 1 | 3 | 2 | 3 | 2 | 1 | 1 | 1 | 3 | 1 | 1 | 1 | 2 | 3 | 1 | 1 | 2 | 3 | 1 | 1 | 2 | 1 | 1 | 3 |
| 2 | 3 | 2 | 2 | 1 | 3 | 1 | 2 | 1 | 2 | 2 | 2 | 3 | 2 | 3 | 1 | 1 | 1 | 2 | 3 | 2 | 3 | 1 | 1 |
| 2 | 3 | 2 | 1 | 2 | 3 | 2 | 2 | 3 | 1 | 3 | 2 | 2 | 2 | 3 | 1 | 1 | 2 | 2 | 3 | 2 | 2 | 1 | 2 |
| 2 | 3 | 1 | 3 | 2 | 3 | 1 | 1 | 2 | 2 | 1 | 3 | 2 | 2 | 1 | 2 | 3 | 2 | 2 | 3 | 2 | 2 | 1 | 2 |
| 3 | 1 | 1 | 3 | 1 | 1 | 1 | 3 | 1 | 1 | 1 | 2 | 3 | 1 | 3 | 1 | 1 | 1 | 3 | 1 | 2 | 2 | 1 | 2 |
| 2 | 2 | 1 | 1 | 3 | 2 | 1 | 1 | 3 | 2 | 2 | 3 | 2 | 3 | 2 | 2 | 3 | 1 | 2 | 1 | 2 | 2 | 1 | 3 |
| 1 | 2 | 3 | 1 | 2 | 3 | 2 | 3 | 2 | 2 | 2 | 3 | 1 | 2 | 2 | 2 | 3 | 1 | 1 | 2 | 2 | 3 | 1 | 1 |
| 1 | 1 | 3 | 2 | 1 | 1 | 3 | 2 | 3 | 1 | 1 | 1 | 2 | 2 | 3 | 2 | 2 | 3 | 2 | 2 | 2 | 3 | 1 | 1 |
| 1 | 2 | 3 | 1 | 1 | 3 | 2 | 3 | 2 | 1 | 1 | 1 | 3 | 2 | 2 | 2 | 3 | 1 | 1 | 1 | 3 | 1 | 1 | 1 |
| 1 | 3 | 1 | 3 | 1 | 3 | 2 | 1 | 1 | 3 | 1 | 2 | 1 | 1 | 2 | 2 | 3 | 2 | 1 | 2 | 1 | 3 | 2 | 1 |
| 2 | 2 | 2 | 1 | 2 | 3 | 1 | 3 | 1 | 2 | 1 | 3 | 1 | 2 | 3 | 1 | 1 | 1 | 2 | 1 | 1 | 3 | 2 | 3 |
| 1 | 3 | 1 | 1 | 1 | 2 | 2 | 1 | 3 | 2 | 1 | 3 | 2 | 1 | 1 | 2 | 3 | 1 | 2 | 2 | 2 | 3 | 2 | 3 |
| 3 | 1 | 2 | 2 | 2 | 3 | 1 | 3 | 1 | 2 | 2 | 3 | 1 | 1 | 2 | 3 | 1 | 3 | 1 | 1 | 2 | 1 | 2 | 1 |
| 3 | 1 | 2 | 2 | 1 | 3 | 1 | 1 | 1 | 3 | 1 | 2 | 3 | 1 | 1 | 2 | 1 | 1 | 1 | 3 | 1 | 2 | 3 | 1 |
| 2 | 1 | 3 | 1 | 2 | 1 | 3 | 1 | 1 | 1 | 3 | 2 | 1 | 2 | 1 | 2 | 3 | 2 | 2 | 3 | 2 | 1 | 3 | 2 |
| 3 | 1 | 1 | 3 | 1 | 2 | 1 | 3 | 2 | 1 | 1 | 1 | 3 | 2 | 1 | 1 | 1 | 3 | 2 | 1 | 1 | 3 | 2 | 2 |
| 1 | 1 | 1 | 2 | 3 | 2 | 3 | 2 | 3 | 2 | 2 | 2 | 1 | 3 | 2 | 1 | 3 | 2 | 2 | 3 | 2 | 1 | 1 | 1 |
| 2 | 2 | 3 | 2 | 2 | 3 | 1 | 1 | 3 | 2 | 1 | 1 | 3 | 1 | 3 | 1 | 2 | 3 | 1 | 1 | 2 | 1 | 1 | 1 |
| 2 | 1 | 2 | 2 | 2 | 3 | 1 | 3 | 1 | 3 | 1 | 1 | 1 | 3 | 1 | 1 | 1 | 3 | 1 | 3 | 2 | 2 | 2 | 1 |
| 2 | 1 | 2 | 2 | 2 | 1 | 3 | 2 | 3 | 1 | 2 | 3 | 1 | 1 | 2 | 2 | 2 | 3 | 2 | 3 | 1 | 2 | 3 | 2 |
| 2 | 2 | 1 | 2 | 1 | 3 | 2 | 3 | 1 | 2 | 3 | 1 | 2 | 3 | 1 | 2 | 1 | 1 | 3 | 2 | 2 | 3 | 1 | 2 |
| 2 | 1 | 1 | 1 | 3 | 1 | 2 | 1 | 1 | 2 | 2 | 3 | 2 | 1 | 3 | 1 | 1 | 1 | 3 | 2 | 1 | 3 | 2 | 3 |
| 3 | 2 | 2 | 2 | 1 | 3 | 2 | 1 | 2 | 2 | 3 | 1 | 2 | 1 | 2 | 2 | 3 | 2 | 3 | 2 | 3 | 2 | 1 | 1 |
| 3 | 2 | 3 | 2 | 2 | 3 | 2 | 3 | 1 | 1 | 2 | 1 | 1 | 3 | 1 | 2 | 2 | 3 | 1 | 1 | 1 | 2 | 1 | 2 |
| 1 | 1 | 1 | 3 | 1 | 1 | 1 | 3 | 2 | 1 | 2 | 1 | 1 | 1 | 3 | 2 | 3 | 1 | 3 | 1 | 2 | 1 | 3 | 1 |
| 2 | 1 | 2 | 2 | 2 | 3 | 2 | 1 | 1 | 3 | 1 | 1 | 3 | 2 | 3 | 2 | 1 | 3 | 1 | 2 | 1 | 2 | 2 | 3 |
| 2 | 1 | 3 | 1 | 1 | 3 | 1 | 2 | 3 | 1 | 1 | 1 | 2 | 2 | 3 | 2 | 3 | 1 | 2 | 2 | 2 | 3 | 2 | 2 |
| 1 | 2 | 1 | 1 | 2 | 1 | 3 | 2 | 1 | 1 | 3 | 2 | 3 | 1 | 1 | 2 | 3 | 1 | 2 | 3 | 1 | 3 | 1 | 2 |
| 1 | 1 | 2 | 3 | 2 | 3 | 1 | 1 | 2 | 1 | 1 | 3 | 1 | 2 | 1 | 1 | 1 | 3 | 2 | 3 | 2 | 3 | 2 | 1 |
| 1 | 2 | 2 | 3 | 1 | 1 | 3 | 1 | 2 | 1 | 1 | 1 | 3 | 1 | 2 | 3 | 2 | 2 | 3 | 2 | 2 | 2 | 1 | 3 |
| 2 | 3 | 1 | 1 | 1 | 2 | 1 | 3 | 1 | 1 | 3 | 2 | 3 | 1 | 3 | 1 | 2 | 2 | 1 | 2 | 1 | 3 | 2 | 1 |
| 1 | 3 | 2 | 2 | 1 | 2 | 2 | 3 | 2 | 3 | 1 | 1 | 1 | 3 | 1 | 3 | 2 | 2 | 2 | 1 | 2 | 3 | 1 | 2 |
| 1 | 1 | 1 | 2 | 1 | 3 | 2 | 1 | 3 | 2 | 3 | 1 | 2 | 1 | 3 | 1 | 3 | 1 | 1 | 3 | 1 | 2 | 2 | 2 |
| 1 | 3 | 2 | 3 | 2 | 1 | 2 | 3 | 1 | 1 | 3 | 2 | 3 | 2 | 1 | 1 | 2 | 1 | 1 | 3 | 1 | 2 | 2 | 1 |
| 2 | 3 | 1 | 2 | 2 | 1 | 1 | 3 | 1 | 2 | 2 | 3 | 2 | 3 | 2 | 1 | 3 | 2 | 3 | 2 | 2 | 1 | 2 | 1 |
| 1 | 3 | 2 | 2 | 2 | 1 | 2 | 3 | 1 | 2 | 1 | 2 | 2 | 2 | 3 | 2 | 1 | 3 | 1 | 2 | 3 | 1 | 3 | 2 |
| 2 | 1 | 2 | 3 | 2 | 3 | 2 | 1 | 2 | 3 | 1 | 1 | 3 | 1 | 2 | 2 | 1 | 2 | 1 | 3 | 2 | 2 | 1 | 3 |
| 3 | 1 | 1 | 1 | 2 | 2 | 3 | 2 | 2 | 3 | 2 | 1 | 2 | 1 | 3 | 1 | 3 | 2 | 3 | 1 | 2 | 1 | 2 | 1 |
| 2 | 1 | 3 | 1 | 1 | 1 | 2 | 1 | 3 | 2 | 2 | 2 | 1 | 1 | 3 | 2 | 1 | 2 | 1 | 3 | 2 | 3 | 2 | 3 |
| 2 | 3 | 1 | 2 | 2 | 2 | 1 | 3 | 1 | 2 | 3 | 2 | 2 | 2 | 1 | 2 | 2 | 3 | 2 | 3 | 1 | 3 | 1 | 1 |
| 1 | 1 | 3 | 2 | 2 | 3 | 1 | 2 | 1 | 2 | 2 | 2 | 3 | 2 | 2 | 3 | 2 | 2 | 1 | 3 | 1 | 2 | 3 | 1 |
| 2 | 3 | 1 | 2 | 3 | 2 | 3 | 1 | 2 | 1 | 1 | 2 | 3 | 1 | 3 | 1 | 1 | 2 | 1 | 1 | 1 | 3 | 1 | 1 |
| 1 | 1 | 1 | 3 | 2 | 2 | 2 | 1 | 3 | 2 | 2 | 2 | 3 | 2 | 1 | 2 | 2 | 1 | 3 | 2 | 1 | 3 | 1 | 3 |
| 1 | 3 | 2 | 2 | 2 | 3 | 1 | 2 | 3 | 2 | 3 | 1 | 2 | 1 | 3 | 2 | 1 | 1 | 1 | 2 | 1 | 3 | 1 | 1 |
| 1 | 1 | 3 | 2 | 3 | 2 | 2 | 1 | 2 | 2 | 3 | 1 | 1 | 2 | 3 | 2 | 3 | 1 | 2 | 3 | 2 | 2 | 1 | 2 |
| 1 | 1 | 1 | 2 | 2 | 3 | 1 | 1 | 3 | 2 | 3 | 2 | 3 | 1 | 2 | 1 | 1 | 2 | 3 | 2 | 2 | 2 | 3 | 2 |
| 3 | 2 | 2 | 2 | 1 | 3 | 2 | 3 | 1 | 2 | 2 | 1 | 1 | 1 | 3 | 1 | 2 | 1 | 3 | 1 | 2 | 2 | 1 | 3 |
| 1 | 2 | 1 | 1 | 3 | 2 | 3 | 2 | 1 | 2 | 1 | 1 | 3 | 1 | 3 | 1 | 1 | 3 | 2 | 3 | 2 | 2 | 1 | 1 |
| 1 | 2 | 3 | 1 | 1 | 2 | 2 | 2 | 3 | 2 | 2 | 2 | 3 | 2 | 3 | 1 | 2 | 3 | 1 | 1 | 3 | 2 | 2 | 1 |
| 1 | 1 | 1 | 3 | 1 | 1 | 2 | 2 | 3 | 1 | 3 | 1 | 1 | 1 | 2 | 3 | 1 | 1 | 1 | 3 | 2 | 2 | 1 | 3 |
| 1 | 3 | 2 | 3 | 2 | 1 | 1 | 3 | 1 | 3 | 2 | 1 | 2 | 1 | 1 | 1 | 3 | 2 | 1 | 2 | 2 | 2 | 3 | 1 |
| 3 | 1 | 1 | 2 | 2 | 1 | 1 | 3 | 1 | 2 | 2 | 3 | 2 | 2 | 1 | 2 | 1 | 2 | 3 | 2 | 3 | 1 | 3 | 2 |
| 2 | 1 | 2 | 3 | 1 | 1 | 1 | 3 | 2 | 3 | 2 | 2 | 3 | 2 | 2 | 2 | 1 | 1 | 3 | 2 | 1 | 1 | 3 | 1 |
| 2 | 1 | 1 | 1 | 3 | 2 | 1 | 1 | 1 | 2 | 3 | 2 | 2 | 1 | 2 | 3 | 2 | 3 | 1 | 3 | 1 | 3 | 1 | 1 |
| 1 | 1 | 1 | 3 | 1 | 2 | 1 | 2 | 2 | 3 | 1 | 2 | 2 | 3 | 1 | 3 | 1 | 2 | 1 | 3 | 1 | 3 | 2 | 2 |
| 1 | 1 | 3 | 2 | 3 | 1 | 2 | 1 | 2 | 3 | 1 | 1 | 2 | 1 | 2 | 3 | 2 | 3 | 1 | 3 | 1 | 1 | 1 | 2 |
| 1 | 1 | 1 | 2 | 1 | 3 | 1 | 3 | 2 | 2 | 2 | 3 | 2 | 2 | 1 | 1 | 2 | 3 | 2 | 1 | 1 | 3 | 2 | 3 |
| 3 | 1 | 2 | 2 | 2 | 1 | 3 | 1 | 2 | 3 | 1 | 3 | 2 | 2 | 1 | 1 | 3 | 1 | 1 | 2 | 2 | 2 | 1 | 3 |
| 2 | 2 | 3 | 2 | 1 | 1 | 1 | 2 | 3 | 1 | 3 | 2 | 3 | 2 | 3 | 1 | 1 | 2 | 1 | 2 | 2 | 3 | 2 | 1 |
| 1 | 3 | 2 | 1 | 3 | 2 | 3 | 2 | 1 | 2 | 2 | 2 | 3 | 1 | 3 | 1 | 2 | 1 | 1 | 2 | 1 | 3 | 1 | 1 |
| 2 | 3 | 1 | 3 | 2 | 2 | 1 | 1 | 1 | 3 | 1 | 3 | 2 | 2 | 3 | 2 | 2 | 3 | 1 | 2 | 1 | 2 | 2 | 2 |
| 1 | 1 | 1 | 3 | 1 | 3 | 2 | 3 | 2 | 1 | 2 | 2 | 1 | 3 | 1 | 1 | 1 | 2 | 1 | 3 | 2 | 2 | 2 | 3 |
| 3 | 2 | 2 | 2 | 1 | 3 | 2 | 2 | 1 | 2 | 2 | 2 | 3 | 1 | 2 | 3 | 1 | 3 | 1 | 2 | 1 | 1 | 2 | 3 |
| 1 | 1 | 3 | 2 | 3 | 2 | 1 | 1 | 1 | 2 | 3 | 1 | 1 | 2 | 1 | 1 | 1 | 3 | 1 | 3 | 2 | 2 | 3 | 2 |
| 1 | 1 | 2 | 1 | 1 | 1 | 3 | 2 | 3 | 1 | 3 | 2 | 1 | 3 | 1 | 1 | 3 | 2 | 3 | 2 | 1 | 1 | 2 | 2 |
| 2 | 1 | 2 | 2 | 3 | 1 | 3 | 2 | 2 | 2 | 3 | 2 | 3 | 2 | 1 | 1 | 1 | 3 | 1 | 1 | 3 | 1 | 2 | 1 |
| 2 | 2 | 2 | 1 | 2 | 1 | 3 | 2 | 2 | 3 | 2 | 2 | 3 | 2 | 3 | 2 | 2 | 3 | 1 | 1 | 1 | 3 | 2 | 2 |
| 1 | 2 | 3 | 1 | 1 | 1 | 2 | 1 | 2 | 3 | 1 | 2 | 2 | 3 | 2 | 3 | 2 | 2 | 2 | 3 | 2 | 2 | 3 | 2 |
| 1 | 1 | 1 | 3 | 1 | 3 | 1 | 2 | 3 | 2 | 1 | 1 | 1 | 3 | 2 | 3 | 1 | 3 | 2 | 2 | 1 | 2 | 2 | 1 |
| 2 | 2 | 3 | 1 | 1 | 3 | 1 | 1 | 1 | 3 | 2 | 2 | 1 | 3 | 1 | 2 | 3 | 1 | 2 | 3 | 1 | 1 | 2 | 2 |
| 1 | 2 | 3 | 2 | 2 | 1 | 2 | 2 | 2 | 3 | 2 | 2 | 2 | 1 | 3 | 2 | 2 | 2 | 3 | 2 | 3 | 2 | 3 | 1 |
| 1 | 1 | 1 | 2 | 1 | 2 | 3 | 1 | 1 | 2 | 2 | 2 | 3 | 1 | 1 | 3 | 1 | 3 | 1 | 1 | 3 | 2 | 3 | 1 |
| 3 | 1 | 2 | 2 | 1 | 3 | 1 | 2 | 1 | 2 | 1 | 3 | 1 | 1 | 2 | 1 | 2 | 2 | 3 | 1 | 1 | 3 | 1 | 3 |
| 2 | 2 | 1 | 3 | 1 | 1 | 2 | 1 | 1 | 3 | 1 | 3 | 1 | 1 | 1 | 2 | 3 | 2 | 1 | 2 | 3 | 2 | 3 | 2 |
| 2 | 2 | 2 | 1 | 2 | 3 | 1 | 1 | 1 | 3 | 1 | 3 | 1 | 1 | 3 | 2 | 3 | 2 | 1 | 2 | 2 | 1 | 2 | 3 |
| 3 | 2 | 1 | 1 | 3 | 2 | 1 | 2 | 2 | 1 | 1 | 3 | 2 | 3 | 2 | 3 | 1 | 2 | 2 | 2 | 1 | 3 | 2 | 1 |
| 1 | 2 | 1 | 1 | 1 | 3 | 1 | 3 | 1 | 1 | 3 | 2 | 1 | 1 | 1 | 3 | 1 | 3 | 2 | 1 | 1 | 1 | 3 | 2 |
| 1 | 2 | 2 | 3 | 2 | 2 | 1 | 1 | 2 | 2 | 3 | 1 | 1 | 3 | 2 | 3 | 2 | 1 | 2 | 3 | 1 | 1 | 1 | 3 |
| 2 | 1 | 2 | 1 | 2 | 1 | 3 | 2 | 2 | 3 | 1 | 3 | 2 | 2 | 3 | 1 | 3 | 2 | 1 | 1 | 3 | 1 | 2 | 2 |
| 2 | 1 | 3 | 1 | 2 | 3 | 1 | 3 | 1 | 2 | 1 | 2 | 1 | 2 | 3 | 1 | 1 | 1 | 3 | 1 | 2 | 1 | 3 | 2 |
| 1 | 2 | 1 | 1 | 3 | 1 | 1 | 3 | 1 | 2 | 3 | 1 | 2 | 2 | 2 | 3 | 2 | 3 | 2 | 1 | 1 | 1 | 2 | 3 |
| 2 | 2 | 1 | 3 | 2 | 1 | 1 | 2 | 1 | 1 | 3 | 1 | 1 | 1 | 3 | 1 | 2 | 3 | 1 | 1 | 3 | 1 | 3 | 1 |
| 3 | 1 | 2 | 2 | 2 | 3 | 2 | 3 | 1 | 3 | 2 | 1 | 1 | 1 | 3 | 2 | 1 | 1 | 1 | 2 | 1 | 3 | 1 | 1 |
| 1 | 1 | 1 | 2 | 1 | 3 | 1 | 2 | 3 | 2 | 1 | 3 | 1 | 1 | 2 | 2 | 2 | 3 | 2 | 3 | 2 | 3 | 2 | 2 |
| 3 | 1 | 1 | 1 | 2 | 2 | 1 | 3 | 2 | 3 | 2 | 2 | 2 | 3 | 2 | 3 | 2 | 3 | 2 | 1 | 2 | 2 | 1 | 2 |
| 1 | 2 | 2 | 2 | 3 | 1 | 3 | 2 | 1 | 2 | 3 | 1 | 2 | 1 | 3 | 1 | 1 | 3 | 1 | 2 | 2 | 3 | 2 | 2 |
| 1 | 2 | 1 | 3 | 1 | 3 | 2 | 2 | 3 | 1 | 1 | 3 | 2 | 1 | 2 | 3 | 2 | 1 | 1 | 1 | 3 | 2 | 2 | 2 |
| 2 | 1 | 1 | 2 | 2 | 2 | 3 | 2 | 3 | 1 | 1 | 2 | 3 | 2 | 2 | 3 | 2 | 2 | 1 | 2 | 2 | 3 | 2 | 3 |
| 2 | 2 | 1 | 3 | 2 | 2 | 2 | 1 | 2 | 3 | 1 | 3 | 1 | 3 | 2 | 3 | 1 | 3 | 1 | 2 | 2 | 2 | 1 | 1 |
| 3 | 2 | 2 | 3 | 2 | 2 | 1 | 3 | 1 | 3 | 2 | 3 | 2 | 2 | 2 | 1 | 2 | 3 | 1 | 1 | 1 | 2 | 2 | 2 |
| 2 | 2 | 2 | 1 | 2 | 2 | 3 | 2 | 3 | 1 | 2 | 3 | 2 | 3 | 1 | 1 | 1 | 2 | 1 | 1 | 3 | 1 | 3 | 1 |
| 3 | 2 | 1 | 1 | 3 | 2 | 1 | 1 | 2 | 1 | 2 | 3 | 1 | 2 | 1 | 3 | 2 | 3 | 1 | 2 | 2 | 1 | 1 | 3 |
| 2 | 3 | 1 | 3 | 1 | 2 | 3 | 1 | 2 | 3 | 2 | 1 | 2 | 1 | 2 | 3 | 2 | 1 | 3 | 2 | 1 | 1 | 2 | 1 |
| 1 | 1 | 2 | 2 | 3 | 1 | 3 | 1 | 1 | 1 | 3 | 1 | 3 | 1 | 2 | 1 | 1 | 1 | 2 | 3 | 1 | 2 | 1 | 3 |
| 2 | 2 | 2 | 3 | 1 | 1 | 3 | 2 | 3 | 1 | 2 | 3 | 2 | 2 | 1 | 3 | 1 | 1 | 2 | 3 | 2 | 2 | 2 | 1 |
| 1 | 3 | 2 | 2 | 3 | 2 | 2 | 3 | 2 | 3 | 2 | 1 | 1 | 2 | 2 | 3 | 2 | 2 | 1 | 3 | 2 | 1 | 1 | 1 |
| 1 | 2 | 1 | 3 | 2 | 3 | 1 | 3 | 1 | 1 | 3 | 2 | 3 | 1 | 2 | 1 | 1 | 3 | 1 | 2 | 1 | 2 | 2 | 2 |
| 3 | 2 | 3 | 2 | 3 | 1 | 2 | 1 | 1 | 3 | 2 | 1 | 1 | 2 | 2 | 3 | 1 | 3 | 2 | 2 | 1 | 1 | 1 | 2 |
| 2 | 1 | 3 | 2 | 2 | 1 | 2 | 2 | 3 | 2 | 2 | 2 | 3 | 2 | 3 | 1 | 1 | 2 | 2 | 2 | 3 | 1 | 3 | 1 |
| 1 | 2 | 1 | 3 | 2 | 2 | 3 | 1 | 1 | 2 | 1 | 3 | 2 | 1 | 1 | 2 | 2 | 2 | 3 | 1 | 1 | 3 | 1 | 3 |
| 1 | 2 | 3 | 2 | 2 | 2 | 3 | 2 | 3 | 2 | 2 | 2 | 3 | 1 | 1 | 2 | 1 | 3 | 1 | 3 | 1 | 1 | 2 | 1 |
| 2 | 3 | 1 | 2 | 1 | 1 | 1 | 3 | 1 | 2 | 1 | 2 | 3 | 1 | 3 | 1 | 3 | 1 | 2 | 2 | 3 | 2 | 1 | 1 |
| 2 | 1 | 1 | 1 | 3 | 1 | 2 | 3 | 1 | 3 | 1 | 2 | 3 | 2 | 2 | 3 | 2 | 2 | 1 | 1 | 1 | 3 | 2 | 2 |
| 1 | 1 | 3 | 2 | 3 | 1 | 1 | 1 | 2 | 2 | 2 | 3 | 2 | 1 | 1 | 3 | 1 | 1 | 2 | 2 | 1 | 3 | 2 | 3 |
| 3 | 1 | 1 | 1 | 2 | 3 | 1 | 3 | 1 | 3 | 2 | 2 | 1 | 2 | 2 | 3 | 1 | 2 | 1 | 3 | 2 | 2 | 2 | 1 |
| 2 | 2 | 2 | 3 | 2 | 1 | 1 | 1 | 2 | 3 | 1 | 3 | 1 | 2 | 1 | 2 | 1 | 3 | 2 | 3 | 2 | 2 | 1 | 3 |
| 3 | 2 | 2 | 1 | 1 | 2 | 2 | 3 | 2 | 3 | 1 | 2 | 1 | 2 | 2 | 2 | 3 | 1 | 2 | 2 | 1 | 3 | 2 | 3 |
| 1 | 3 | 1 | 3 | 2 | 3 | 2 | 2 | 3 | 1 | 2 | 1 | 1 | 1 | 3 | 1 | 2 | 3 | 2 | 2 | 2 | 1 | 2 | 1 |
| 1 | 1 | 2 | 2 | 3 | 2 | 3 | 1 | 3 | 1 | 1 | 1 | 2 | 2 | 3 | 1 | 2 | 1 | 1 | 3 | 1 | 1 | 3 | 1 |
| 2 | 2 | 1 | 1 | 1 | 3 | 1 | 3 | 1 | 1 | 2 | 2 | 3 | 1 | 3 | 1 | 1 | 3 | 1 | 3 | 1 | 1 | 1 | 2 |
| 2 | 2 | 3 | 2 | 2 | 1 | 3 | 1 | 1 | 3 | 1 | 1 | 2 | 2 | 3 | 1 | 1 | 2 | 3 | 2 | 1 | 2 | 3 | 2 |
| 1 | 3 | 2 | 2 | 1 | 1 | 3 | 1 | 2 | 1 | 2 | 3 | 2 | 3 | 2 | 3 | 1 | 2 | 3 | 2 | 2 | 2 | 1 | 1 |
| 2 | 3 | 1 | 3 | 2 | 2 | 1 | 2 | 3 | 2 | 2 | 3 | 2 | 1 | 1 | 2 | 1 | 3 | 1 | 1 | 1 | 2 | 2 | 3 |
| 2 | 2 | 1 | 3 | 1 | 2 | 1 | 1 | 3 | 2 | 2 | 2 | 1 | 3 | 1 | 3 | 1 | 2 | 2 | 3 | 1 | 3 | 1 | 1 |
| 1 | 2 | 3 | 1 | 3 | 2 | 1 | 1 | 2 | 1 | 1 | 3 | 1 | 3 | 2 | 1 | 2 | 2 | 2 | 3 | 1 | 1 | 3 | 2 |
| 2 | 3 | 2 | 2 | 2 | 1 | 1 | 3 | 2 | 3 | 2 | 1 | 1 | 2 | 3 | 1 | 2 | 2 | 2 | 3 | 2 | 2 | 1 | 3 |
| 2 | 2 | 3 | 1 | 1 | 3 | 1 | 1 | 3 | 1 | 2 | 2 | 3 | 2 | 2 | 1 | 2 | 2 | 3 | 2 | 2 | 3 | 1 | 1 |
| 2 | 1 | 2 | 1 | 3 | 1 | 1 | 1 | 3 | 1 | 2 | 2 | 1 | 1 | 1 | 3 | 1 | 3 | 2 | 3 | 1 | 1 | 2 | 3 |
| 2 | 1 | 1 | 1 | 2 | 2 | 3 | 2 | 2 | 1 | 3 | 1 | 1 | 1 | 2 | 2 | 2 | 3 | 1 | 3 | 2 | 3 | 2 | 3 |
| 1 | 2 | 2 | 3 | 2 | 2 | 1 | 3 | 2 | 3 | 2 | 3 | 2 | 2 | 1 | 2 | 2 | 3 | 1 | 2 | 2 | 1 | 2 | 3 |
| 3 | 1 | 3 | 1 | 1 | 2 | 2 | 1 | 2 | 3 | 2 | 3 | 2 | 3 | 1 | 1 | 2 | 1 | 2 | 1 | 3 | 1 | 1 | 1 |
| 2 | 2 | 3 | 1 | 2 | 2 | 3 | 1 | 2 | 1 | 1 | 1 | 3 | 2 | 1 | 1 | 1 | 3 | 1 | 3 | 2 | 3 | 2 | 1 |
| 3 | 2 | 3 | 2 | 3 | 2 | 1 | 1 | 1 | 2 | 2 | 3 | 1 | 1 | 2 | 1 | 2 | 3 | 2 | 2 | 1 | 1 | 2 | 3 |
| 1 | 1 | 1 | 3 | 2 | 1 | 1 | 1 | 3 | 1 | 1 | 1 | 3 | 1 | 1 | 3 | 2 | 2 | 2 | 3 | 1 | 1 | 1 | 3 |
| 2 | 2 | 2 | 1 | 3 | 2 | 2 | 3 | 1 | 1 | 3 | 1 | 1 | 2 | 1 | 3 | 1 | 1 | 1 | 3 | 1 | 1 | 1 | 3 |
| 3 | 2 | 3 | 2 | 1 | 1 | 2 | 1 | 1 | 3 | 1 | 3 | 2 | 3 | 1 | 1 | 2 | 1 | 3 | 2 | 1 | 1 | 2 | 2 |
| 2 | 1 | 2 | 2 | 3 | 1 | 1 | 1 | 2 | 1 | 1 | 3 | 1 | 3 | 1 | 3 | 1 | 2 | 2 | 2 | 3 | 2 | 3 | 1 |
| 1 | 2 | 3 | 1 | 3 | 1 | 1 | 1 | 3 | 1 | 1 | 3 | 1 | 1 | 3 | 2 | 2 | 1 | 1 | 3 | 1 | 2 | 2 | 2 |
| 1 | 1 | 3 | 1 | 3 | 2 | 3 | 1 | 3 | 2 | 1 | 2 | 1 | 2 | 2 | 3 | 2 | 2 | 1 | 1 | 1 | 3 | 1 | 1 |
| 2 | 2 | 2 | 3 | 2 | 1 | 1 | 1 | 3 | 2 | 3 | 1 | 2 | 3 | 1 | 2 | 3 | 2 | 1 | 1 | 3 | 1 | 2 | 1 |
| 3 | 1 | 2 | 3 | 2 | 2 | 1 | 2 | 3 | 2 | 3 | 1 | 2 | 3 | 1 | 1 | 1 | 2 | 1 | 2 | 3 | 2 | 1 | 2 |
| 3 | 2 | 1 | 3 | 1 | 1 | 2 | 1 | 1 | 1 | 3 | 2 | 3 | 2 | 2 | 1 | 1 | 1 | 3 | 2 | 3 | 2 | 2 | 1 |
| 1 | 1 | 1 | 3 | 1 | 3 | 2 | 1 | 2 | 3 | 2 | 3 | 2 | 3 | 2 | 1 | 2 | 3 | 1 | 2 | 1 | 2 | 2 | 2 |
| 1 | 1 | 1 | 3 | 1 | 2 | 1 | 1 | 3 | 1 | 3 | 2 | 2 | 1 | 3 | 2 | 1 | 1 | 1 | 2 | 2 | 3 | 2 | 3 |
| 1 | 1 | 3 | 1 | 1 | 2 | 2 | 1 | 3 | 1 | 3 | 1 | 1 | 2 | 1 | 1 | 3 | 2 | 3 | 2 | 3 | 1 | 2 | 1 |
| 3 | 1 | 2 | 1 | 1 | 3 | 1 | 1 | 1 | 3 | 2 | 3 | 1 | 1 | 1 | 2 | 3 | 2 | 1 | 1 | 1 | 2 | 2 | 3 |
| 3 | 1 | 2 | 3 | 1 | 1 | 1 | 3 | 1 | 2 | 3 | 2 | 2 | 2 | 1 | 1 | 1 | 3 | 2 | 2 | 2 | 3 | 2 | 2 |
| 1 | 3 | 2 | 3 | 2 | 1 | 1 | 3 | 2 | 1 | 1 | 2 | 1 | 1 | 3 | 2 | 2 | 2 | 3 | 1 | 3 | 1 | 1 | 1 |
| 3 | 2 | 2 | 3 | 1 | 3 | 1 | 1 | 2 | 2 | 1 | 3 | 1 | 1 | 2 | 2 | 2 | 3 | 1 | 2 | 1 | 1 | 1 | 3 |
| 2 | 2 | 1 | 1 | 3 | 1 | 1 | 1 | 2 | 2 | 2 | 3 | 2 | 1 | 2 | 3 | 2 | 3 | 2 | 2 | 3 | 2 | 2 | 3 |
| 1 | 3 | 1 | 1 | 3 | 1 | 2 | 2 | 2 | 1 | 3 | 1 | 2 | 3 | 1 | 1 | 1 | 2 | 3 | 1 | 3 | 2 | 2 | 2 |
| 2 | 1 | 1 | 3 | 2 | 2 | 2 | 3 | 1 | 3 | 1 | 2 | 1 | 1 | 1 | 3 | 1 | 2 | 3 | 1 | 2 | 1 | 2 | 3 |
| 2 | 3 | 1 | 3 | 1 | 2 | 1 | 3 | 2 | 2 | 2 | 3 | 2 | 1 | 1 | 2 | 1 | 2 | 3 | 2 | 2 | 2 | 3 | 2 |
| 1 | 3 | 2 | 2 | 2 | 3 | 1 | 1 | 1 | 2 | 2 | 3 | 2 | 1 | 1 | 3 | 2 | 2 | 2 | 3 | 1 | 2 | 3 | 1 |
| 2 | 1 | 3 | 1 | 1 | 2 | 2 | 3 | 1 | 2 | 2 | 1 | 1 | 2 | 3 | 1 | 2 | 3 | 1 | 3 | 2 | 1 | 3 | 2 |
| 1 | 3 | 1 | 3 | 1 | 2 | 2 | 2 | 3 | 2 | 1 | 1 | 2 | 1 | 1 | 3 | 2 | 1 | 2 | 2 | 3 | 1 | 1 | 3 |
| 1 | 2 | 1 | 1 | 2 | 3 | 1 | 2 | 3 | 2 | 1 | 1 | 2 | 3 | 2 | 1 | 1 | 3 | 2 | 1 | 3 | 2 | 3 | 2 |
| 2 | 3 | 1 | 1 | 1 | 2 | 2 | 2 | 3 | 1 | 2 | 3 | 1 | 3 | 1 | 3 | 1 | 2 | 1 | 2 | 3 | 2 | 2 | 1 |
| 2 | 3 | 2 | 3 | 2 | 1 | 1 | 1 | 3 | 2 | 1 | 2 | 1 | 3 | 2 | 2 | 2 | 1 | 2 | 3 | 2 | 2 | 1 | 3 |
| 2 | 3 | 1 | 1 | 2 | 1 | 1 | 3 | 2 | 3 | 1 | 1 | 1 | 2 | 1 | 3 | 1 | 1 | 2 | 3 | 1 | 1 | 2 | 3 |
| 1 | 1 | 1 | 3 | 1 | 1 | 1 | 3 | 1 | 2 | 2 | 3 | 2 | 1 | 1 | 2 | 1 | 1 | 3 | 2 | 1 | 3 | 1 | 3 |
| 1 | 1 | 2 | 3 | 1 | 1 | 1 | 2 | 1 | 3 | 2 | 3 | 2 | 2 | 1 | 1 | 1 | 2 | 3 | 1 | 3 | 2 | 3 | 2 |
| 3 | 2 | 1 | 3 | 1 | 2 | 1 | 1 | 1 | 3 | 1 | 2 | 3 | 2 | 3 | 1 | 1 | 2 | 2 | 1 | 2 | 3 | 1 | 2 |
| 3 | 1 | 2 | 1 | 3 | 2 | 1 | 2 | 1 | 2 | 3 | 2 | 3 | 2 | 3 | 2 | 1 | 2 | 2 | 2 | 3 | 2 | 2 | 2 |
| 1 | 2 | 3 | 2 | 2 | 2 | 3 | 2 | 1 | 3 | 1 | 1 | 1 | 2 | 3 | 2 | 2 | 2 | 3 | 1 | 1 | 3 | 1 | 2 |
| 1 | 1 | 1 | 2 | 2 | 2 | 3 | 2 | 1 | 3 | 1 | 3 | 1 | 3 | 1 | 1 | 1 | 2 | 2 | 2 | 3 | 2 | 2 | 3 |
| 2 | 1 | 3 | 1 | 1 | 2 | 1 | 1 | 3 | 1 | 2 | 2 | 1 | 3 | 2 | 1 | 1 | 3 | 2 | 3 | 2 | 1 | 3 | 1 |
| 2 | 3 | 1 | 2 | 2 | 2 | 1 | 3 | 1 | 3 | 1 | 1 | 1 | 2 | 1 | 2 | 3 | 1 | 3 | 2 | 1 | 3 | 1 | 1 |
| 1 | 1 | 2 | 1 | 3 | 1 | 3 | 2 | 1 | 2 | 3 | 2 | 2 | 3 | 2 | 2 | 2 | 1 | 2 | 3 | 1 | 3 | 1 | 1 |
| 3 | 1 | 2 | 3 | 1 | 2 | 3 | 1 | 1 | 3 | 1 | 3 | 2 | 2 | 2 | 1 | 2 | 2 | 3 | 2 | 1 | 1 | 1 | 2 |
| 1 | 1 | 3 | 2 | 1 | 1 | 1 | 3 | 1 | 1 | 3 | 1 | 1 | 3 | 1 | 1 | 1 | 2 | 3 | 2 | 3 | 2 | 2 | 1 |
| 2 | 2 | 3 | 1 | 1 | 3 | 1 | 1 | 2 | 2 | 1 | 1 | 3 | 2 | 3 | 2 | 2 | 2 | 1 | 3 | 2 | 3 | 2 | 1 |
| 1 | 3 | 1 | 1 | 1 | 3 | 1 | 1 | 2 | 3 | 2 | 2 | 3 | 1 | 2 | 2 | 2 | 1 | 2 | 3 | 1 | 2 | 3 | 2 |
| 3 | 1 | 2 | 2 | 1 | 1 | 1 | 3 | 1 | 3 | 1 | 2 | 3 | 2 | 2 | 3 | 1 | 2 | 2 | 3 | 1 | 1 | 1 | 2 |
| 1 | 1 | 2 | 3 | 1 | 2 | 1 | 1 | 2 | 2 | 3 | 2 | 2 | 3 | 1 | 3 | 1 | 3 | 1 | 3 | 2 | 1 | 1 | 2 |
| 3 | 2 | 2 | 2 | 3 | 2 | 2 | 3 | 1 | 1 | 1 | 3 | 2 | 3 | 2 | 1 | 1 | 1 | 3 | 2 | 1 | 2 | 1 | 2 |
| 2 | 3 | 1 | 3 | 2 | 2 | 1 | 2 | 1 | 2 | 3 | 1 | 3 | 1 | 1 | 1 | 3 | 2 | 3 | 2 | 1 | 1 | 2 | 2 |
| 2 | 2 | 3 | 2 | 3 | 1 | 3 | 1 | 1 | 1 | 3 | 1 | 1 | 3 | 2 | 1 | 2 | 1 | 2 | 1 | 3 | 1 | 1 | 2 |
| 3 | 2 | 1 | 1 | 3 | 2 | 2 | 2 | 1 | 3 | 1 | 3 | 2 | 2 | 1 | 2 | 1 | 3 | 1 | 3 | 2 | 2 | 2 | 1 |
| 3 | 1 | 2 | 1 | 3 | 1 | 2 | 1 | 3 | 1 | 2 | 1 | 1 | 3 | 2 | 2 | 1 | 1 | 2 | 2 | 3 | 1 | 1 | 3 |
| 1 | 3 | 1 | 3 | 1 | 2 | 3 | 1 | 2 | 2 | 3 | 2 | 2 | 2 | 1 | 2 | 3 | 2 | 1 | 2 | 2 | 1 | 2 | 3 |
| 1 | 1 | 1 | 3 | 2 | 2 | 1 | 1 | 3 | 1 | 1 | 1 | 2 | 2 | 3 | 2 | 1 | 3 | 2 | 3 | 1 | 2 | 1 | 3 |
| 2 | 2 | 2 | 3 | 1 | 2 | 1 | 2 | 2 | 3 | 2 | 2 | 2 | 3 | 2 | 3 | 1 | 3 | 2 | 3 | 2 | 1 | 2 | 1 |
| 1 | 2 | 2 | 2 | 3 | 2 | 1 | 3 | 1 | 1 | 1 | 3 | 2 | 2 | 3 | 2 | 2 | 1 | 2 | 3 | 1 | 3 | 2 | 2 |
| 3 | 1 | 2 | 2 | 2 | 3 | 1 | 3 | 2 | 1 | 1 | 3 | 2 | 2 | 2 | 1 | 2 | 1 | 3 | 1 | 2 | 3 | 1 | 1 |
| 1 | 1 | 3 | 1 | 2 | 1 | 1 | 1 | 3 | 2 | 3 | 1 | 3 | 2 | 2 | 3 | 1 | 2 | 2 | 2 | 1 | 3 | 1 | 2 |
| 3 | 1 | 2 | 1 | 2 | 2 | 3 | 2 | 1 | 1 | 3 | 1 | 2 | 1 | 2 | 3 | 2 | 2 | 3 | 2 | 1 | 1 | 1 | 3 |
| 3 | 2 | 1 | 1 | 3 | 1 | 3 | 2 | 3 | 2 | 1 | 2 | 2 | 3 | 2 | 1 | 1 | 3 | 2 | 2 | 1 | 1 | 2 | 2 |
| 3 | 2 | 3 | 2 | 3 | 1 | 2 | 2 | 1 | 3 | 2 | 1 | 1 | 2 | 3 | 1 | 1 | 3 | 2 | 1 | 2 | 2 | 2 | 1 |
| 3 | 2 | 1 | 1 | 3 | 1 | 1 | 1 | 3 | 1 | 2 | 2 | 1 | 1 | 3 | 2 | 3 | 2 | 2 | 1 | 3 | 2 | 1 | 1 |
| 1 | 3 | 2 | 1 | 3 | 1 | 1 | 1 | 3 | 2 | 2 | 3 | 1 | 1 | 1 | 2 | 2 | 3 | 1 | 2 | 2 | 1 | 2 | 3 |
| 2 | 1 | 1 | 3 | 1 | 3 | 1 | 1 | 3 | 2 | 2 | 3 | 1 | 3 | 2 | 1 | 1 | 2 | 3 | 2 | 1 | 2 | 2 | 2 |
| 3 | 2 | 2 | 1 | 1 | 3 | 1 | 1 | 1 | 2 | 1 | 3 | 2 | 1 | 3 | 1 | 2 | 1 | 1 | 3 | 2 | 3 | 1 | 1 |
| 2 | 1 | 1 | 3 | 2 | 1 | 1 | 1 | 2 | 2 | 3 | 1 | 1 | 1 | 3 | 2 | 3 | 2 | 1 | 2 | 1 | 3 | 2 | 3 |
| 1 | 1 | 3 | 1 | 2 | 3 | 2 | 1 | 2 | 3 | 2 | 2 | 2 | 1 | 2 | 2 | 3 | 2 | 2 | 3 | 2 | 3 | 2 | 1 |
| 1 | 2 | 2 | 2 | 1 | 3 | 1 | 1 | 2 | 1 | 2 | 1 | 3 | 2 | 3 | 1 | 1 | 3 | 1 | 3 | 1 | 2 | 1 | 3 |
| 3 | 2 | 2 | 1 | 2 | 3 | 1 | 1 | 1 | 3 | 1 | 3 | 2 | 1 | 2 | 3 | 2 | 3 | 2 | 2 | 1 | 1 | 1 | 2 |
| 2 | 1 | 2 | 2 | 1 | 2 | 3 | 2 | 3 | 1 | 1 | 3 | 1 | 1 | 3 | 1 | 1 | 2 | 3 | 1 | 2 | 2 | 1 | 3 |
| 2 | 1 | 1 | 2 | 1 | 1 | 3 | 2 | 2 | 3 | 1 | 1 | 3 | 1 | 3 | 1 | 1 | 2 | 2 | 3 | 2 | 2 | 3 | 2 |
| 2 | 3 | 1 | 2 | 3 | 2 | 2 | 2 | 3 | 1 | 2 | 3 | 2 | 1 | 1 | 2 | 2 | 3 | 2 | 2 | 1 | 1 | 1 | 3 |
| 3 | 2 | 3 | 1 | 1 | 1 | 3 | 1 | 2 | 2 | 2 | 3 | 1 | 3 | 2 | 2 | 2 | 3 | 2 | 1 | 2 | 1 | 1 | 2 |
| 1 | 3 | 1 | 3 | 1 | 1 | 2 | 1 | 2 | 1 | 3 | 1 | 2 | 2 | 3 | 1 | 3 | 1 | 2 | 2 | 2 | 3 | 2 | 2 |
| 2 | 2 | 2 | 3 | 1 | 3 | 1 | 2 | 3 | 2 | 3 | 1 | 2 | 3 | 1 | 2 | 1 | 1 | 1 | 3 | 2 | 2 | 1 | 1 |
| 3 | 2 | 2 | 3 | 2 | 1 | 1 | 1 | 2 | 2 | 3 | 2 | 1 | 3 | 2 | 1 | 1 | 1 | 3 | 1 | 1 | 3 | 2 | 1 |
| 3 | 2 | 3 | 2 | 2 | 1 | 2 | 3 | 1 | 2 | 3 | 2 | 2 | 3 | 2 | 2 | 2 | 3 | 2 | 1 | 2 | 2 | 1 | 2 |
| 1 | 2 | 2 | 1 | 2 | 2 | 3 | 2 | 3 | 2 | 1 | 3 | 1 | 2 | 3 | 2 | 1 | 2 | 2 | 1 | 1 | 3 | 1 | 3 |
| 3 | 2 | 2 | 1 | 3 | 1 | 1 | 1 | 3 | 1 | 2 | 2 | 2 | 1 | 3 | 1 | 1 | 3 | 2 | 2 | 1 | 3 | 2 | 2 |
| 2 | 2 | 3 | 2 | 3 | 2 | 1 | 2 | 2 | 1 | 1 | 3 | 1 | 3 | 1 | 3 | 2 | 3 | 1 | 1 | 1 | 2 | 1 | 2 |
| 3 | 2 | 2 | 2 | 1 | 1 | 3 | 1 | 2 | 1 | 3 | 1 | 1 | 1 | 3 | 1 | 3 | 2 | 3 | 1 | 2 | 2 | 2 | 1 |
| 1 | 1 | 2 | 3 | 1 | 3 | 1 | 1 | 1 | 2 | 1 | 3 | 1 | 2 | 1 | 3 | 2 | 2 | 1 | 2 | 2 | 3 | 2 | 3 |
| 2 | 3 | 1 | 1 | 2 | 2 | 3 | 1 | 1 | 2 | 1 | 1 | 3 | 1 | 1 | 2 | 2 | 2 | 3 | 2 | 2 | 3 | 2 | 3 |
| 1 | 1 | 2 | 1 | 1 | 3 | 1 | 2 | 2 | 3 | 1 | 1 | 2 | 2 | 1 | 3 | 2 | 3 | 1 | 3 | 2 | 1 | 1 | 3 |
| 1 | 1 | 2 | 3 | 2 | 2 | 2 | 3 | 1 | 3 | 1 | 3 | 1 | 2 | 2 | 2 | 1 | 3 | 2 | 1 | 1 | 1 | 3 | 1 |
| 1 | 3 | 2 | 2 | 2 | 1 | 3 | 1 | 1 | 2 | 1 | 3 | 1 | 1 | 1 | 2 | 3 | 2 | 3 | 2 | 2 | 2 | 3 | 1 |
| 2 | 1 | 2 | 1 | 1 | 3 | 2 | 1 | 1 | 3 | 2 | 3 | 2 | 2 | 1 | 1 | 3 | 1 | 2 | 2 | 2 | 3 | 1 | 3 |
| 3 | 2 | 1 | 3 | 2 | 3 | 1 | 1 | 2 | 1 | 1 | 3 | 2 | 2 | 1 | 3 | 2 | 3 | 2 | 2 | 1 | 1 | 2 | 1 |
| 1 | 1 | 3 | 2 | 3 | 2 | 3 | 2 | 2 | 1 | 1 | 1 | 3 | 2 | 1 | 1 | 1 | 2 | 3 | 2 | 1 | 3 | 1 | 2 |
| 1 | 3 | 1 | 3 | 1 | 2 | 3 | 2 | 2 | 2 | 1 | 2 | 3 | 2 | 2 | 3 | 2 | 3 | 1 | 1 | 2 | 2 | 1 | 1 |
| 1 | 3 | 2 | 2 | 3 | 1 | 1 | 2 | 1 | 2 | 2 | 3 | 1 | 2 | 3 | 1 | 2 | 1 | 1 | 3 | 1 | 1 | 3 | 1 |
| 2 | 3 | 1 | 1 | 2 | 3 | 2 | 3 | 1 | 3 | 1 | 2 | 3 | 2 | 2 | 2 | 1 | 3 | 1 | 1 | 2 | 1 | 1 | 2 |
| 1 | 1 | 2 | 1 | 1 | 2 | 3 | 1 | 2 | 3 | 2 | 1 | 1 | 3 | 2 | 2 | 2 | 3 | 1 | 3 | 2 | 2 | 2 | 3 |
| 1 | 1 | 1 | 3 | 1 | 3 | 2 | 3 | 1 | 1 | 2 | 1 | 3 | 1 | 1 | 1 | 2 | 1 | 1 | 3 | 1 | 3 | 1 | 1 |
| 1 | 1 | 2 | 1 | 1 | 1 | 3 | 2 | 2 | 1 | 2 | 2 | 3 | 1 | 3 | 1 | 3 | 1 | 3 | 2 | 2 | 2 | 1 | 3 |
| 1 | 3 | 2 | 1 | 3 | 2 | 3 | 2 | 2 | 3 | 2 | 1 | 3 | 2 | 2 | 2 | 1 | 3 | 2 | 1 | 2 | 1 | 2 | 1 |
| 3 | 2 | 1 | 1 | 3 | 1 | 1 | 2 | 3 | 2 | 1 | 2 | 2 | 1 | 3 | 1 | 2 | 1 | 2 | 2 | 2 | 3 | 2 | 3 |
| 3 | 1 | 2 | 1 | 1 | 1 | 2 | 3 | 2 | 2 | 2 | 3 | 1 | 2 | 1 | 1 | 1 | 3 | 2 | 1 | 3 | 2 | 2 | 3 |
| 1 | 2 | 1 | 3 | 2 | 1 | 2 | 3 | 2 | 1 | 2 | 3 | 2 | 3 | 2 | 3 | 1 | 1 | 3 | 1 | 2 | 2 | 2 | 1 |
| 1 | 2 | 3 | 1 | 1 | 2 | 3 | 2 | 1 | 3 | 1 | 3 | 2 | 3 | 1 | 2 | 2 | 1 | 3 | 2 | 2 | 2 | 1 | 1 |
| 3 | 2 | 1 | 3 | 2 | 1 | 2 | 2 | 2 | 1 | 3 | 2 | 3 | 1 | 2 | 3 | 2 | 1 | 1 | 3 | 1 | 1 | 2 | 1 |
| 1 | 3 | 1 | 1 | 2 | 2 | 3 | 2 | 1 | 2 | 2 | 3 | 1 | 1 | 3 | 1 | 1 | 3 | 1 | 1 | 2 | 1 | 2 | 3 |
| 2 | 2 | 2 | 1 | 2 | 1 | 3 | 1 | 1 | 2 | 2 | 3 | 1 | 3 | 1 | 3 | 1 | 1 | 3 | 2 | 2 | 1 | 1 | 3 |
| 1 | 1 | 1 | 3 | 2 | 1 | 3 | 2 | 1 | 3 | 1 | 3 | 1 | 2 | 2 | 2 | 3 | 1 | 3 | 1 | 1 | 2 | 2 | 1 |
| 2 | 2 | 2 | 1 | 1 | 1 | 3 | 1 | 1 | 1 | 3 | 2 | 1 | 2 | 2 | 3 | 2 | 1 | 1 | 3 | 1 | 3 | 2 | 3 |
| 1 | 1 | 1 | 2 | 2 | 3 | 1 | 3 | 1 | 1 | 1 | 3 | 2 | 3 | 1 | 1 | 2 | 3 | 1 | 1 | 3 | 2 | 2 | 2 |
| 1 | 1 | 3 | 1 | 1 | 1 | 2 | 1 | 1 | 3 | 2 | 1 | 2 | 3 | 1 | 2 | 1 | 3 | 2 | 1 | 3 | 2 | 1 | 3 |
| 1 | 2 | 2 | 2 | 3 | 1 | 1 | 2 | 2 | 3 | 2 | 1 | 2 | 2 | 3 | 2 | 1 | 3 | 2 | 2 | 2 | 3 | 2 | 3 |
| 1 | 1 | 3 | 1 | 3 | 1 | 1 | 2 | 1 | 1 | 2 | 3 | 2 | 1 | 3 | 1 | 3 | 1 | 2 | 1 | 2 | 1 | 1 | 3 |
| 2 | 3 | 2 | 3 | 2 | 1 | 1 | 2 | 1 | 3 | 2 | 2 | 3 | 2 | 2 | 1 | 1 | 2 | 3 | 1 | 3 | 2 | 1 | 1 |
| 2 | 1 | 2 | 1 | 3 | 2 | 2 | 3 | 2 | 1 | 3 | 2 | 2 | 2 | 1 | 3 | 1 | 2 | 3 | 1 | 1 | 2 | 3 | 2 |
| 1 | 2 | 2 | 3 | 2 | 3 | 2 | 2 | 1 | 3 | 1 | 1 | 2 | 3 | 1 | 2 | 3 | 2 | 2 | 1 | 1 | 2 | 1 | 3 |
| 3 | 2 | 2 | 2 | 3 | 2 | 1 | 2 | 1 | 3 | 2 | 1 | 2 | 2 | 2 | 3 | 1 | 2 | 2 | 3 | 1 | 2 | 3 | 2 |
| 1 | 3 | 1 | 3 | 2 | 1 | 1 | 1 | 3 | 2 | 1 | 2 | 3 | 1 | 3 | 2 | 2 | 1 | 2 | 3 | 1 | 1 | 2 | 1 |
| 3 | 1 | 1 | 1 | 3 | 2 | 2 | 2 | 1 | 1 | 3 | 2 | 3 | 1 | 2 | 3 | 2 | 1 | 2 | 1 | 2 | 2 | 3 | 2 |
| 2 | 2 | 1 | 1 | 1 | 2 | 3 | 1 | 2 | 1 | 1 | 1 | 3 | 1 | 3 | 2 | 1 | 3 | 2 | 3 | 1 | 1 | 3 | 2 |
| 2 | 2 | 1 | 1 | 1 | 2 | 3 | 2 | 3 | 2 | 3 | 1 | 3 | 1 | 1 | 3 | 1 | 2 | 3 | 1 | 1 | 2 | 1 | 1 |
| 1 | 2 | 2 | 2 | 3 | 2 | 1 | 2 | 1 | 1 | 1 | 3 | 2 | 3 | 1 | 1 | 3 | 1 | 1 | 3 | 1 | 3 | 1 | 1 |
| 2 | 3 | 1 | 2 | 2 | 1 | 3 | 2 | 1 | 2 | 2 | 2 | 3 | 2 | 3 | 1 | 1 | 3 | 1 | 3 | 1 | 2 | 2 | 2 |
| 2 | 2 | 2 | 3 | 1 | 1 | 2 | 3 | 1 | 1 | 1 | 2 | 2 | 3 | 1 | 2 | 3 | 1 | 2 | 1 | 3 | 1 | 2 | 3 |
| 1 | 3 | 1 | 3 | 2 | 1 | 1 | 3 | 1 | 2 | 2 | 1 | 1 | 3 | 1 | 1 | 2 | 1 | 1 | 3 | 1 | 1 | 1 | 3 |
| 1 | 2 | 2 | 3 | 1 | 1 | 2 | 2 | 3 | 1 | 3 | 1 | 1 | 3 | 2 | 3 | 1 | 1 | 3 | 2 | 1 | 1 | 1 | 2 |
| 2 | 2 | 2 | 1 | 3 | 1 | 3 | 1 | 1 | 3 | 2 | 1 | 2 | 2 | 3 | 2 | 2 | 2 | 3 | 1 | 1 | 1 | 3 | 1 |
| 2 | 1 | 1 | 1 | 3 | 2 | 3 | 1 | 1 | 1 | 3 | 1 | 2 | 2 | 2 | 3 | 1 | 1 | 1 | 2 | 3 | 1 | 2 | 3 |
| 3 | 1 | 1 | 1 | 3 | 2 | 2 | 1 | 3 | 1 | 3 | 1 | 1 | 1 | 2 | 3 | 2 | 1 | 3 | 1 | 1 | 1 | 2 | 2 |
| 3 | 2 | 3 | 1 | 1 | 2 | 1 | 1 | 2 | 3 | 1 | 1 | 3 | 1 | 1 | 3 | 2 | 2 | 1 | 2 | 3 | 2 | 2 | 1 |
| 2 | 2 | 3 | 2 | 3 | 1 | 1 | 2 | 1 | 1 | 1 | 3 | 2 | 1 | 3 | 1 | 2 | 3 | 2 | 3 | 2 | 2 | 1 | 2 |
| 2 | 2 | 1 | 2 | 1 | 2 | 3 | 1 | 2 | 1 | 2 | 3 | 1 | 3 | 2 | 2 | 2 | 3 | 2 | 3 | 2 | 2 | 3 | 1 |
| 2 | 2 | 3 | 1 | 2 | 2 | 2 | 3 | 2 | 3 | 2 | 3 | 1 | 3 | 2 | 1 | 2 | 2 | 1 | 3 | 2 | 2 | 1 | 2 |
| 1 | 1 | 1 | 3 | 2 | 3 | 1 | 2 | 2 | 1 | 1 | 3 | 2 | 2 | 1 | 3 | 2 | 2 | 2 | 3 | 1 | 3 | 1 | 2 |
| 2 | 2 | 3 | 2 | 1 | 2 | 2 | 2 | 3 | 2 | 1 | 2 | 1 | 1 | 2 | 3 | 2 | 2 | 3 | 1 | 1 | 3 | 1 | 3 |
| 3 | 2 | 2 | 2 | 3 | 1 | 1 | 1 | 2 | 2 | 1 | 3 | 2 | 3 | 2 | 3 | 1 | 3 | 1 | 1 | 1 | 2 | 1 | 2 |
| 1 | 1 | 2 | 3 | 2 | 2 | 3 | 1 | 3 | 1 | 2 | 2 | 3 | 1 | 2 | 1 | 1 | 2 | 3 | 2 | 2 | 3 | 1 | 1 |
| 2 | 1 | 3 | 2 | 1 | 3 | 2 | 1 | 3 | 2 | 1 | 2 | 2 | 3 | 2 | 2 | 3 | 2 | 1 | 1 | 2 | 1 | 1 | 3 |
| 3 | 2 | 2 | 3 | 2 | 1 | 1 | 2 | 2 | 2 | 3 | 1 | 3 | 2 | 3 | 2 | 2 | 1 | 3 | 2 | 2 | 1 | 2 | 2 |
| 2 | 3 | 1 | 1 | 2 | 1 | 2 | 3 | 1 | 2 | 1 | 3 | 2 | 2 | 1 | 3 | 2 | 1 | 1 | 2 | 2 | 3 | 2 | 3 |
| 2 | 3 | 1 | 2 | 1 | 3 | 2 | 1 | 2 | 3 | 2 | 2 | 2 | 3 | 2 | 3 | 1 | 2 | 2 | 1 | 1 | 1 | 3 | 1 |
| 3 | 1 | 2 | 3 | 2 | 1 | 2 | 1 | 1 | 1 | 3 | 1 | 3 | 2 | 1 | 2 | 3 | 2 | 2 | 1 | 2 | 1 | 1 | 3 |
| 1 | 3 | 2 | 3 | 1 | 3 | 1 | 2 | 2 | 2 | 1 | 3 | 1 | 1 | 3 | 1 | 2 | 3 | 2 | 2 | 1 | 2 | 2 | 1 |
| 1 | 2 | 3 | 1 | 3 | 1 | 1 | 2 | 2 | 2 | 3 | 2 | 2 | 1 | 1 | 1 | 3 | 1 | 3 | 1 | 1 | 1 | 3 | 2 |
| 1 | 1 | 1 | 3 | 1 | 1 | 2 | 2 | 1 | 3 | 2 | 1 | 2 | 3 | 1 | 2 | 1 | 3 | 1 | 2 | 3 | 1 | 3 | 1 |
| 2 | 1 | 3 | 1 | 3 | 2 | 2 | 3 | 2 | 1 | 2 | 1 | 3 | 2 | 2 | 2 | 1 | 2 | 1 | 3 | 2 | 2 | 3 | 1 |
| 3 | 2 | 1 | 3 | 1 | 1 | 2 | 3 | 1 | 2 | 2 | 3 | 2 | 2 | 2 | 1 | 3 | 1 | 1 | 3 | 1 | 2 | 2 | 2 |
| 3 | 2 | 2 | 2 | 1 | 2 | 3 | 2 | 2 | 2 | 3 | 1 | 3 | 1 | 1 | 3 | 1 | 3 | 2 | 2 | 1 | 2 | 2 | 2 |
| 2 | 1 | 3 | 1 | 1 | 3 | 2 | 2 | 2 | 3 | 1 | 1 | 1 | 3 | 2 | 2 | 1 | 2 | 2 | 3 | 1 | 2 | 2 | 3 |
| 3 | 1 | 2 | 3 | 1 | 1 | 3 | 1 | 3 | 2 | 1 | 2 | 2 | 2 | 3 | 2 | 2 | 1 | 2 | 1 | 2 | 3 | 2 | 1 |
| 3 | 1 | 2 | 3 | 1 | 1 | 2 | 1 | 2 | 1 | 3 | 2 | 1 | 1 | 3 | 2 | 1 | 2 | 2 | 3 | 1 | 3 | 2 | 1 |
| 2 | 1 | 3 | 2 | 3 | 1 | 2 | 3 | 1 | 1 | 1 | 2 | 2 | 2 | 3 | 1 | 3 | 1 | 2 | 1 | 3 | 1 | 2 | 1 |
| 3 | 1 | 1 | 1 | 3 | 1 | 1 | 1 | 2 | 2 | 3 | 1 | 1 | 3 | 1 | 3 | 2 | 2 | 2 | 3 | 1 | 2 | 1 | 2 |
| 1 | 2 | 2 | 2 | 3 | 1 | 3 | 2 | 1 | 2 | 2 | 2 | 3 | 2 | 3 | 2 | 1 | 2 | 2 | 3 | 1 | 1 | 2 | 3 |
| 1 | 2 | 3 | 1 | 3 | 2 | 2 | 3 | 1 | 1 | 1 | 2 | 2 | 2 | 3 | 1 | 1 | 3 | 2 | 1 | 2 | 2 | 3 | 2 |
| 2 | 2 | 1 | 1 | 2 | 1 | 3 | 2 | 3 | 1 | 3 | 1 | 3 | 1 | 3 | 2 | 1 | 2 | 1 | 2 | 3 | 2 | 1 | 1 |
| 1 | 2 | 2 | 1 | 1 | 3 | 1 | 3 | 1 | 3 | 2 | 3 | 1 | 3 | 2 | 1 | 1 | 1 | 2 | 3 | 2 | 1 | 1 | 1 |
| 1 | 1 | 3 | 1 | 1 | 2 | 1 | 3 | 1 | 2 | 3 | 1 | 3 | 1 | 2 | 2 | 1 | 3 | 1 | 1 | 1 | 2 | 1 | 3 |
| 1 | 3 | 2 | 2 | 2 | 1 | 1 | 1 | 3 | 1 | 3 | 2 | 2 | 1 | 3 | 1 | 1 | 2 | 2 | 3 | 1 | 1 | 1 | 3 |
| 3 | 2 | 1 | 1 | 3 | 1 | 2 | 2 | 2 | 3 | 2 | 2 | 3 | 1 | 1 | 2 | 1 | 1 | 1 | 3 | 1 | 1 | 3 | 1 |
| 1 | 3 | 1 | 3 | 1 | 1 | 1 | 3 | 1 | 1 | 3 | 2 | 2 | 1 | 1 | 1 | 3 | 2 | 3 | 1 | 2 | 1 | 2 | 2 |
| 2 | 1 | 1 | 2 | 1 | 3 | 1 | 3 | 1 | 1 | 3 | 1 | 3 | 1 | 2 | 3 | 2 | 1 | 2 | 3 | 1 | 1 | 2 | 1 |
| 2 | 2 | 1 | 2 | 2 | 1 | 3 | 2 | 3 | 1 | 2 | 1 | 1 | 3 | 2 | 3 | 1 | 1 | 3 | 2 | 2 | 2 | 1 | 3 |
| 1 | 2 | 1 | 1 | 2 | 3 | 2 | 1 | 1 | 1 | 3 | 1 | 2 | 3 | 1 | 3 | 2 | 2 | 2 | 1 | 2 | 3 | 1 | 3 |
| 2 | 2 | 3 | 1 | 2 | 2 | 2 | 3 | 1 | 3 | 1 | 3 | 2 | 2 | 3 | 1 | 2 | 1 | 1 | 3 | 1 | 2 | 2 | 2 |
| 1 | 2 | 3 | 1 | 2 | 2 | 1 | 2 | 2 | 3 | 2 | 3 | 2 | 3 | 2 | 1 | 3 | 1 | 1 | 2 | 2 | 1 | 3 | 1 |
| 2 | 1 | 2 | 1 | 1 | 1 | 3 | 1 | 2 | 1 | 2 | 1 | 3 | 2 | 1 | 3 | 1 | 2 | 3 | 1 | 2 | 3 | 2 | 3 |
| 2 | 2 | 2 | 1 | 3 | 2 | 2 | 3 | 1 | 3 | 1 | 2 | 3 | 1 | 1 | 3 | 2 | 2 | 1 | 2 | 2 | 1 | 3 | 1 |
| 1 | 2 | 2 | 3 | 1 | 1 | 2 | 2 | 3 | 1 | 2 | 1 | 2 | 1 | 3 | 2 | 3 | 2 | 1 | 1 | 1 | 3 | 2 | 3 |
| 3 | 1 | 1 | 3 | 1 | 1 | 1 | 3 | 1 | 2 | 2 | 1 | 2 | 2 | 3 | 2 | 1 | 2 | 2 | 3 | 1 | 3 | 2 | 2 |
| 1 | 2 | 2 | 3 | 1 | 3 | 2 | 3 | 2 | 1 | 3 | 2 | 3 | 1 | 2 | 2 | 2 | 1 | 3 | 1 | 1 | 1 | 2 | 1 |
| 1 | 1 | 2 | 1 | 1 | 1 | 3 | 2 | 3 | 2 | 2 | 2 | 1 | 1 | 3 | 1 | 3 | 2 | 1 | 3 | 1 | 3 | 2 | 1 |
| 3 | 2 | 1 | 3 | 1 | 3 | 1 | 2 | 1 | 1 | 2 | 2 | 3 | 1 | 2 | 3 | 2 | 3 | 2 | 1 | 1 | 2 | 2 | 2 |

wherein each of 1 to 3 is a nucleotide base selected to be different from the others of 1 to 3 with the proviso that up to three nucleotide bases of each sequence can be substituted with any nucleotide base provided that:
for any pair of sequences of the set:
M1≦16, M2≦13, M3≦20, M4≦16, and M5≦19 where:
M1 is the maximum number of matches for any alignment in which there are no internal indels;
M2 is the maximum length of a block of matches for any alignment;
M3 is the maximum number of matches for any alignment having a maximum score;
M4 is the maximum sum of the lengths of the longest two blocks of matches for any alignment of maximum score; and
M5 is the maximum sum of the lengths of all the blocks of matches having a length of at least 3, for any alignment of maximum score; wherein:
the score of an alignment is determined according to the equation (A×m)−(B×mm)−(C×(og+eg))−(D×eg)), wherein:
for each of (i) to (iv):
(i) m=6, mm=6, og=0 and eg=6,
(ii) m=6, mm=6, og=0 and eg=1,
(iii) m=6, mm=2, og=5 and eg=1, and
(iv) m=6, mm=6, og=6 and eg=0,
A is the total number of matched pairs of bases in the alignment;
B is the total number of internal mismatched pairs in the alignment;
C is the total number of internal gaps in the alignment; and
D is the total number of internal indels in the alignment minus the total number of internal gaps in the alignment; and
wherein the maximum score is determined separately for each of (i), (ii), (iii) and (iv).
v) a second target nucleic acid, distinct from said first target nucleic acid, and having a fourth region, a fifth region and a sixth region, wherein said fourth region is located adjacent to and downstream from said fifth region, and said fifth region is located adjacent to and downstream from said sixth region, said fifth region having a sequence complementary to said 3′ portion of said sequence selected from the group of sequences listed in step (a)(iv), said sixth region having a sequence complementary to said 5′ portion of the sequence selected from the group of sequences in step (a)(iv);
vi) a third oligonucleotide having a 5′ portion, a central portion and a 3′ portion, said 5′ portion of said third oligonucleotide having a sequence complementary to said fourth region of said second target nucleic acid, said central portion of said third oligonucleotide having a sequence complementary to said fifth region of said second target nucleic acid, and said 3′ portion of said third oligonucleotide having a sequence that is not base paired to either said second target nucleic acid or said first target nucleic acid and is selected from a set of oligonucleotides based on the group of sequences listed in step (a)(iv) such that said sequence selected is distinct from said sequence selected in step (a)(iv);
b) mixing said cleavage means, said first target nucleic acid, said second target nucleic acid, said first, second, and third oligonucleotides to create a reaction mixture under reaction conditions such that at least said 5′ portion of said first oligonucleotide is annealed to said first target nucleic acid and wherein at least said 5′ and central portion of said second oligonucleotide is annealed to said first target nucleic acid so as to create a first cleavage structure and wherein the combined melting temperature of said complementary regions within said 5′ and 3′ portions of said first oligonucleotide when annealed to said first target nucleic acid is greater than the melting temperature of said 5′ and central portion of said first oligonucleotide, wherein cleavage of said first cleavage structure occurs to generate a first non-target cleavage product, and wherein at least said 5′ portion first non-target cleavage product is annealed to said second target nucleic acid and at least said 5′ and central portion of said third oligonucleotide is annealed to said second target nucleic acid so as to create a second cleavage structure and wherein the combined melting temperature of said complementary regions within said 5′ and 3′ portions of said non-target cleavage product when annealed to said second target nucleic acid is greater than the melting temperature of said 5′ and central portion of said third oligonucleotide, wherein cleavage of said second cleavage structure occurs to generate a second non-target cleavage product; and
c) detecting said second non-target cleavage product.

68. The method of claim 67, wherein said first target nucleic acid is genomic DNA and said second target nucleic acid is synthetic DNA.

69. The method of claim 68, wherein said synthetic DNA has at least one hairpin loop.

70. The method of claim 69, wherein the method includes a plurality of said first target nucleic acid sequences, a plurality of first oligonucleotide molecules, a plurality of said second oligonucleotide molecules, a plurality of said second target nucleic acid sequences and a plurality of third oligonucleotide molecules.


SEQUENCE LISTING (1384 sequences)

SEQ ID NO: 1 - DNA, 24 bp, Artificial Sequence
  Description of Artificial SequenceArtificially Synthesized DNA Sequence
aaattgtgaaagattgtttgtgta

SEQ ID NO: 2 - DNA, 24 bp, Artificial Sequence
  Description of Artificial SequenceArtificially Synthesized DNA Sequence
gttagagttaattgtatttgatga

SEQ ID NO: 3 - DNA, 24 bp, Artificial Sequence
  Description of Artificial SequenceArtificially Synthesized DNA Sequence
atgttaaagtaagtgttgaaatgt

SEQ ID NO: 4 - DNA, 24 bp, Artificial Sequence
  Description of Artificial SequenceArtificially Synthesized DNA Sequence
tgatgttagaagtatattgtgaat

SEQ ID NO: 5 - DNA, 24 bp, Artificial Sequence
  Description of Artificial SequenceArtificially Synthesized DNA Sequence
tttgtgtagaatatgtgttgttaa

SEQ ID NO: 6 - DNA, 24 bp, Artificial Sequence
  Description of Artificial SequenceArtificially Synthesized DNA Sequence
ataagtgtaagtgaaataagaaga

SEQ ID NO: 7 - DNA, 24 bp, Artificial Sequence
  Description of Artificial SequenceArtificially Synthesized DNA Sequence
aagagtatttgttgtgagttaaat

SEQ ID NO: 8 - DNA, 24 bp, Artificial Sequence
  Description of Artificial SequenceArtificially Synthesized DNA Sequence
gtgtttatgttatatgtgaagttt

SEQ ID NO: 9 - DNA, 24 bp, Artificial Sequence
  Description of Artificial SequenceArtificially Synthesized DNA Sequence
aaagagaatagaatatgtgtaagt

SEQ ID NO: 10 - DNA, 24 bp, Artificial Sequence
  Description of Artificial SequenceArtificially Synthesized DNA Sequence
tatgaaagagtgagataatgttta

SEQ ID NO: 11 - DNA, 24 bp, Artificial Sequence
  Description of Artificial SequenceArtificially Synthesized DNA Sequence
atgagaaatatgttagaatgtgat

SEQ ID NO: 12 - DNA, 24 bp, Artificial Sequence
  Description of Artificial SequenceArtificially Synthesized DNA Sequence
ttagttgttgatgtttagtagttt

SEQ ID NO: 13 - DNA, 24 bp, Artificial Sequence
  Description of Artificial SequenceArtificially Synthesized DNA Sequence
gtaaagagtataagtttgatgata

SEQ ID NO: 14 - DNA, 24 bp, Artificial Sequence
  Description of Artificial SequenceArtificially Synthesized DNA Sequence
aaagtaagaatgatgtaataagtg

SEQ ID NO: 15 - DNA, 24 bp, Artificial Sequence
  Description of Artificial SequenceArtificially Synthesized DNA Sequence
gtagaaatagtttattgatgattg

SEQ ID NO: 16 - DNA, 24 bp, Artificial Sequence
  Description of Artificial SequenceArtificially Synthesized DNA Sequence
tgtaagtgaaatagtgagttattt

SEQ ID NO: 17 - DNA, 24 bp, Artificial Sequence
  Description of Artificial SequenceArtificially Synthesized DNA Sequence
aaatagatgatataagtgagaatg

SEQ ID NO: 18 - DNA, 24 bp, Artificial Sequence
  Description of Artificial SequenceArtificially Synthesized DNA Sequence
ataagttataagtgttatgtgagt

SEQ ID NO: 19 - DNA, 24 bp, Artificial Sequence
  Description of Artificial SequenceArtificially Synthesized DNA Sequence
tatagataaagagatgatttgttg

SEQ ID NO: 20 - DNA, 24 bp, Artificial Sequence
  Description of Artificial SequenceArtificially Synthesized DNA Sequence
agagttgagaatgtatagtattat

SEQ ID NO: 21 - DNA, 24 bp, Artificial Sequence
  Description of Artificial SequenceArtificially Synthesized DNA Sequence
aagtagtttgtaagaatgattgta

SEQ ID NO: 22 - DNA, 24 bp, Artificial Sequence
  Description of Artificial SequenceArtificially Synthesized DNA Sequence
ttatgaaattgagtgaagattgat

SEQ ID NO: 23 - DNA, 24 bp, Artificial Sequence
  Description of Artificial SequenceArtificially Synthesized DNA Sequence
gtatatgtaaattgttatgttgag

SEQ ID NO: 24 - DNA, 24 bp, Artificial Sequence
  Description of Artificial SequenceArtificially Synthesized DNA Sequence
gaattgtataaagtattagatgtg

SEQ ID NO: 25 - DNA, 24 bp, Artificial Sequence
  Description of Artificial SequenceArtificially Synthesized DNA Sequence
tagatgagattaagtgttatttga

SEQ ID NO: 26 - DNA, 24 bp, Artificial Sequence
  Description of Artificial SequenceArtificially Synthesized DNA Sequence
gttaagtttgtttatgtatagaag

SEQ ID NO: 27 - DNA, 24 bp, Artificial Sequence
  Description of Artificial SequenceArtificially Synthesized DNA Sequence
gagtattagtaaagtgatatgata

SEQ ID NO: 28 - DNA, 24 bp, Artificial Sequence
  Description of Artificial SequenceArtificially Synthesized DNA Sequence
gtgaatgatttagtaaatgattga

SEQ ID NO: 29 - DNA, 24 bp, Artificial Sequence
  Description of Artificial SequenceArtificially Synthesized DNA Sequence
gattgaagttatagaaatgattag

SEQ ID NO: 30 - DNA, 24 bp, Artificial Sequence
  Description of Artificial SequenceArtificially Synthesized DNA Sequence
agtgataaatgttagttgaattgt

SEQ ID NO: 31 - DNA, 24 bp, Artificial Sequence
  Description of Artificial SequenceArtificially Synthesized DNA Sequence
tatatagtaaatgtttgtgtgttg

SEQ ID NO: 32 - DNA, 24 bp, Artificial Sequence
  Description of Artificial SequenceArtificially Synthesized DNA Sequence
ttaagtgttagttatttgttgtag

SEQ ID NO: 33 - DNA, 24 bp, Artificial Sequence
  Description of Artificial SequenceArtificially Synthesized DNA Sequence
gtagtaatatgaagtgagaatata

SEQ ID NO: 34 - DNA, 24 bp, Artificial Sequence
  Description of Artificial SequenceArtificially Synthesized DNA Sequence
tagtgtatagaatgtagatttagt

SEQ ID NO: 35 - DNA, 24 bp, Artificial Sequence
  Description of Artificial SequenceArtificially Synthesized DNA Sequence
ttgtagattagatgtgtttgtaaa

SEQ ID NO: 36 - DNA, 24 bp, Artificial Sequence
  Description of Artificial SequenceArtificially Synthesized DNA Sequence
tagtatagagtagagatgatattt

SEQ ID NO: 37 - DNA, 24 bp, Artificial Sequence
  Description of Artificial SequenceArtificially Synthesized DNA Sequence
attgtgaaagaaagagaagaaatt

SEQ ID NO: 38 - DNA, 24 bp, Artificial Sequence
  Description of Artificial SequenceArtificially Synthesized DNA Sequence
tgtgagaattaagattaagaatgt

SEQ ID NO: 39 - DNA, 24 bp, Artificial Sequence
  Description of Artificial SequenceArtificially Synthesized DNA Sequence
atattagttaagaaagaagagttg

SEQ ID NO: 40 - DNA, 24 bp, Artificial Sequence
  Description of Artificial SequenceArtificially Synthesized DNA Sequence
ttgtagttgagaaatatgtagttt

SEQ ID NO: 41 - DNA, 24 bp, Artificial Sequence
  Description of Artificial SequenceArtificially Synthesized DNA Sequence
tagagttgttaaagagtgtaaata

SEQ ID NO: 42 - DNA, 24 bp, Artificial Sequence
  Description of Artificial SequenceArtificially Synthesized DNA Sequence
gttatgatgtgtataagtaatatg

SEQ ID NO: 43 - DNA, 24 bp, Artificial Sequence
  Description of Artificial SequenceArtificially Synthesized DNA Sequence
tttgttagaatgagaagatttatg

SEQ ID NO: 44 - DNA, 24 bp, Artificial Sequence
  Description of Artificial SequenceArtificially Synthesized DNA Sequence
agtatagtttaaagaagtagtaga

SEQ ID NO: 45 - DNA, 24 bp, Artificial Sequence
  Description of Artificial SequenceArtificially Synthesized DNA Sequence
gtgagatatagatttagaaagtaa

SEQ ID NO: 46 - DNA, 24 bp, Artificial Sequence
  Description of Artificial SequenceArtificially Synthesized DNA Sequence
ttgtttatagtgaagtgaatagta

SEQ ID NO: 47 - DNA, 24 bp, Artificial Sequence
  Description of Artificial SequenceArtificially Synthesized DNA Sequence
aagtaagtagtaatagtgtgttaa

SEQ ID NO: 48 - DNA, 24 bp, Artificial Sequence
  Description of Artificial SequenceArtificially Synthesized DNA Sequence
atttgtgagttatgaaagataaga

SEQ ID NO: 49 - DNA, 24 bp, Artificial Sequence
  Description of Artificial SequenceArtificially Synthesized DNA Sequence
gaaagtagagaataaagataagaa

SEQ ID NO: 50 - DNA, 24 bp, Artificial Sequence
  Description of Artificial SequenceArtificially Synthesized DNA Sequence
atttaagattgttaagagtagaag

SEQ ID NO: 51 - DNA, 24 bp, Artificial Sequence
  Description of Artificial SequenceArtificially Synthesized DNA Sequence
gtttaaagattgtaagaatgtgta

SEQ ID NO: 52 - DNA, 24 bp, Artificial Sequence
  Description of Artificial SequenceArtificially Synthesized DNA Sequence
tttgtgaagatgaagtatttgtat

SEQ ID NO: 53 - DNA, 24 bp, Artificial Sequence
  Description of Artificial SequenceArtificially Synthesized DNA Sequence
tgtgtttagaatttagtatgtgta

SEQ ID NO: 54 - DNA, 24 bp, Artificial Sequence
  Description of Artificial SequenceArtificially Synthesized DNA Sequence
gataatgattatagaaagtgtttg

SEQ ID NO: 55 - DNA, 24 bp, Artificial Sequence
  Description of Artificial SequenceArtificially Synthesized DNA Sequence
gttatttgtaagttaagatagtag

SEQ ID NO: 56 - DNA, 24 bp, Artificial Sequence
  Description of Artificial SequenceArtificially Synthesized DNA Sequence
agtttattgaaagagtttgaatag

SEQ ID NO: 57 - DNA, 24 bp, Artificial Sequence
  Description of Artificial SequenceArtificially Synthesized DNA Sequence
ttgtgtttattgtgtagtttaaag

SEQ ID NO: 58 - DNA, 24 bp, Artificial Sequence
  Description of Artificial SequenceArtificially Synthesized DNA Sequence
attgtgagaagatatgaaagttat

SEQ ID NO: 59 - DNA, 24 bp, Artificial Sequence
  Description of Artificial SequenceArtificially Synthesized DNA Sequence
tgagaatgtaaagaatgtttattg

SEQ ID NO: 60 - DNA, 24 bp, Artificial Sequence
  Description of Artificial SequenceArtificially Synthesized DNA Sequence
atgtgaaagttatgatgttaattg

SEQ ID NO: 61 - DNA, 24 bp, Artificial Sequence
  Description of Artificial SequenceArtificially Synthesized DNA Sequence
gtttagtattagttgttaagattg

SEQ ID NO: 62 - DNA, 24 bp, Artificial Sequence
  Description of Artificial SequenceArtificially Synthesized DNA Sequence
gattgatatttgaatgtttgtttg

SEQ ID NO: 63 - DNA, 24 bp, Artificial Sequence
  Description of Artificial SequenceArtificially Synthesized DNA Sequence
tgaattgaaagtgtaatgttgtat

SEQ ID NO: 64 - DNA, 24 bp, Artificial Sequence
  Description of Artificial SequenceArtificially Synthesized DNA Sequence
gattgtattgttgagaatagaata

SEQ ID NO: 65 - DNA, 24 bp, Artificial Sequence
  Description of Artificial SequenceArtificially Synthesized DNA Sequence
aaatttgagatttgtgatagagta

SEQ ID NO: 66 - DNA, 24 bp, Artificial Sequence
  Description of Artificial SequenceArtificially Synthesized DNA Sequence
gtaattagatttgtttgttgttgt

SEQ ID NO: 67 - DNA, 24 bp, Artificial Sequence
  Description of Artificial SequenceArtificially Synthesized DNA Sequence
gtttgtattgttagtgaatatagt

SEQ ID NO: 68 - DNA, 24 bp, Artificial Sequence
  Description of Artificial SequenceArtificially Synthesized DNA Sequence
atgtagtagtagatgtttatgaat

SEQ ID NO: 69 - DNA, 24 bp, Artificial Sequence
  Description of Artificial SequenceArtificially Synthesized DNA Sequence
tgtttaaagatgattgaagaaatg

SEQ ID NO: 70 - DNA, 24 bp, Artificial Sequence
  Description of Artificial SequenceArtificially Synthesized DNA Sequence
tgtgataatgatgttatttgtgta

SEQ ID NO: 71 - DNA, 24 bp, Artificial Sequence
  Description of Artificial SequenceArtificially Synthesized DNA Sequence
atagttgtgagaatttgtaattag

SEQ ID NO: 72 - DNA, 24 bp, Artificial Sequence
  Description of Artificial SequenceArtificially Synthesized DNA Sequence
atagatgtaagagaaattgtgaaa

SEQ ID NO: 73 - DNA, 24 bp, Artificial Sequence
  Description of Artificial SequenceArtificially Synthesized DNA Sequence
agattaagagaagttaatagagta

SEQ ID NO: 74 - DNA, 24 bp, Artificial Sequence
  Description of Artificial SequenceArtificially Synthesized DNA Sequence
gaagtaaattgtgaatgaaagaaa

SEQ ID NO: 75 - DNA, 24 bp, Artificial Sequence
  Description of Artificial SequenceArtificially Synthesized DNA Sequence
aatgtaagaaagaagattgttgta

SEQ ID NO: 76 - DNA, 24 bp, Artificial Sequence
  Description of Artificial SequenceArtificially Synthesized DNA Sequence
tttgatttatgtgttatgttgagt

SEQ ID NO: 77 - DNA, 24 bp, Artificial Sequence
  Description of Artificial SequenceArtificially Synthesized DNA Sequence
gtattgagaaatttgaagaatgaa

SEQ ID NO: 78 - DNA, 24 bp, Artificial Sequence
  Description of Artificial SequenceArtificially Synthesized DNA Sequence
gaattgtatgaaatgaattgtaag

SEQ ID NO: 79 - DNA, 24 bp, Artificial Sequence
  Description of Artificial SequenceArtificially Synthesized DNA Sequence
tattgtagaagtaaagttagaagt

SEQ ID NO: 80 - DNA, 24 bp, Artificial Sequence
  Description of Artificial SequenceArtificially Synthesized DNA Sequence
tttatgtaatgataagtgtagttg

SEQ ID NO: 81 - DNA, 24 bp, Artificial Sequence
  Description of Artificial SequenceArtificially Synthesized DNA Sequence
atatagttgaaattgtgatagtgt

SEQ ID NO: 82 - DNA, 24 bp, Artificial Sequence
  Description of Artificial SequenceArtificially Synthesized DNA Sequence
ataagaaattagagagttgtaaag

SEQ ID NO: 83 - DNA, 24 bp, Artificial Sequence
  Description of Artificial SequenceArtificially Synthesized DNA Sequence
gaattgtgaaatgtgattgatata

SEQ ID NO: 84 - DNA, 24 bp, Artificial Sequence
  Description of Artificial SequenceArtificially Synthesized DNA Sequence
aaataagtagtttaatgagagaag

SEQ ID NO: 85 - DNA, 24 bp, Artificial Sequence
  Description of Artificial SequenceArtificially Synthesized DNA Sequence
gattaaagaagtaagtgaatgttt

SEQ ID NO: 86 - DNA, 24 bp, Artificial Sequence
  Description of Artificial SequenceArtificially Synthesized DNA Sequence
tatgtgtgttgtttagtgttatta

SEQ ID NO: 87 - DNA, 24 bp, Artificial Sequence
  Description of Artificial SequenceArtificially Synthesized DNA Sequence
gagttatatgtagttagagttata

SEQ ID NO: 88 - DNA, 24 bp, Artificial Sequence
  Description of Artificial SequenceArtificially Synthesized DNA Sequence
gaaagaaagaagtgttaagttaaa

SEQ ID NO: 89 - DNA, 24 bp, Artificial Sequence
  Description of Artificial SequenceArtificially Synthesized DNA Sequence
tagtattagtaagtatgtgattgt

SEQ ID NO: 90 - DNA, 24 bp, Artificial Sequence
  Description of Artificial SequenceArtificially Synthesized DNA Sequence
ttgtgtgattgaatattgtgaaat

SEQ ID NO: 91 - DNA, 24 bp, Artificial Sequence
  Description of Artificial SequenceArtificially Synthesized DNA Sequence
atgtgaaagagttaagtgattaaa

SEQ ID NO: 92 - DNA, 24 bp, Artificial Sequence
  Description of Artificial SequenceArtificially Synthesized DNA Sequence
gattgaatgattgagatatgtaaa

SEQ ID NO: 93 - DNA, 24 bp, Artificial Sequence
  Description of Artificial SequenceArtificially Synthesized DNA Sequence
aagatgatagttaagtgtaagtta

SEQ ID NO: 94 - DNA, 24 bp, Artificial Sequence
  Description of Artificial SequenceArtificially Synthesized DNA Sequence
tagttgttattgagaatttagaag

SEQ ID NO: 95 - DNA, 24 bp, Artificial Sequence
  Description of Artificial SequenceArtificially Synthesized DNA Sequence
tttatagtgaattatgagtgaaag

SEQ ID NO: 96 - DNA, 24 bp, Artificial Sequence
  Description of Artificial SequenceArtificially Synthesized DNA Sequence
gatagatttagaatgaattaagtg

SEQ ID NO: 97 - DNA, 24 bp, Artificial Sequence
  Description of Artificial SequenceArtificially Synthesized DNA Sequence
tttgaagaagagatttgaaattga

SEQ ID NO: 98 - DNA, 24 bp, Artificial Sequence
  Description of Artificial SequenceArtificially Synthesized DNA Sequence
atgaataagagttgataaatgtga

SEQ ID NO: 99 - DNA, 24 bp, Artificial Sequence
  Description of Artificial SequenceArtificially Synthesized DNA Sequence
tgtttatgtagtgtagattgaatt

SEQ ID NO: 100 - DNA, 24 bp, Artificial Sequence
  Description of Artificial SequenceArtificially Synthesized DNA Sequence
tttaagtgagttatagaagtagta

SEQ ID NO: 101 - DNA, 24 bp, Artificial Sequence
  Description of Artificial SequenceArtificially Synthesized DNA Sequence
gatttatgtgtttgaagttaagat

SEQ ID NO: 102 - DNA, 24 bp, Artificial Sequence
  Description of Artificial SequenceArtificially Synthesized DNA Sequence
tagttagagaaagtgataaagtta

SEQ ID NO: 103 - DNA, 24 bp, Artificial Sequence
  Description of Artificial SequenceArtificially Synthesized DNA Sequence
gtaatgataatgaagtgtatatag

SEQ ID NO: 104 - DNA, 24 bp, Artificial Sequence
  Description of Artificial SequenceArtificially Synthesized DNA Sequence
aatgaagtgttagtatagatagta

SEQ ID NO: 105 - DNA, 24 bp, Artificial Sequence
  Description of Artificial SequenceArtificially Synthesized DNA Sequence
taaattgagtttgtttgattgtag

SEQ ID NO: 106 - DNA, 24 bp, Artificial Sequence
  Description of Artificial SequenceArtificially Synthesized DNA Sequence
taatgaagaataagtatgagtgtt

SEQ ID NO: 107 - DNA, 24 bp, Artificial Sequence
  Description of Artificial SequenceArtificially Synthesized DNA Sequence
aaatgtaatagtgttgttagttag

SEQ ID NO: 108 - DNA, 24 bp, Artificial Sequence
  Description of Artificial SequenceArtificially Synthesized DNA Sequence
agagttagtgaaatgttgttaaat

SEQ ID NO: 109 - DNA, 24 bp, Artificial Sequence
  Description of Artificial SequenceArtificially Synthesized DNA Sequence
gaaatagaaatgtattgtttgtga

SEQ ID NO: 110 - DNA, 24 bp, Artificial Sequence
  Description of Artificial SequenceArtificially Synthesized DNA Sequence
agttataagtttgtgagaattaag

SEQ ID NO: 111 - DNA, 24 bp, Artificial Sequence
  Description of Artificial SequenceArtificially Synthesized DNA Sequence
gagtttatagttagaatatgttgt

SEQ ID NO: 112 - DNA, 24 bp, Artificial Sequence
  Description of Artificial SequenceArtificially Synthesized DNA Sequence
agagttattagaagaagatttaag

SEQ ID NO: 113 - DNA, 24 bp, Artificial Sequence
  Description of Artificial SequenceArtificially Synthesized DNA Sequence
gagttaatgaaataagtatttgtg

SEQ ID NO: 114 - DNA, 24 bp, Artificial Sequence
  Description of Artificial SequenceArtificially Synthesized DNA Sequence
atgatgaatagttgaagtatatag

SEQ ID NO: 115 - DNA, 24 bp, Artificial Sequence
  Description of Artificial SequenceArtificially Synthesized DNA Sequence
atagatatgagatgaaagttagta

SEQ ID NO: 116 - DNA, 24 bp, Artificial Sequence
  Description of Artificial SequenceArtificially Synthesized DNA Sequence
tatgtaaagaaagtgaaagaagaa

SEQ ID NO: 117 - DNA, 24 bp, Artificial Sequence
  Description of Artificial SequenceArtificially Synthesized DNA Sequence
tgaatgtagaaatgaatgttgaaa

SEQ ID NO: 118 - DNA, 24 bp, Artificial Sequence
  Description of Artificial SequenceArtificially Synthesized DNA Sequence
aattgaatagtgtgtgagtttaat

SEQ ID NO: 119 - DNA, 24 bp, Artificial Sequence
  Description of Artificial SequenceArtificially Synthesized DNA Sequence
agatattgtttgattaatgaagag

SEQ ID NO: 120 - DNA, 24 bp, Artificial Sequence
  Description of Artificial SequenceArtificially Synthesized DNA Sequence
aaagttgtaaagttgaagataaag

SEQ ID NO: 121 - DNA, 24 bp, Artificial Sequence
  Description of Artificial SequenceArtificially Synthesized DNA Sequence
gttaagagattatgagatgtatta

SEQ ID NO: 122 - DNA, 24 bp, Artificial Sequence
  Description of Artificial SequenceArtificially Synthesized DNA Sequence
agaagatataagaagattgaattg

SEQ ID NO: 123 - DNA, 24 bp, Artificial Sequence
  Description of Artificial SequenceArtificially Synthesized DNA Sequence
gtagaaatttgaattgatgtgaaa

SEQ ID NO: 124 - DNA, 24 bp, Artificial Sequence
  Description of Artificial SequenceArtificially Synthesized DNA Sequence
aagagtagattgataagtatatga

SEQ ID NO: 125 - DNA, 24 bp, Artificial Sequence
  Description of Artificial SequenceArtificially Synthesized DNA Sequence
tgatatagtagtgaagaaataagt

SEQ ID NO: 126 - DNA, 24 bp, Artificial Sequence
  Description of Artificial SequenceArtificially Synthesized DNA Sequence
agataatgatgagaaatgaagata

SEQ ID NO: 127 - DNA, 24 bp, Artificial Sequence
  Description of Artificial SequenceArtificially Synthesized DNA Sequence
atgtgaaagtatttgtgatatagt

SEQ ID NO: 128 - DNA, 24 bp, Artificial Sequence
  Description of Artificial SequenceArtificially Synthesized DNA Sequence
aataagagaattgatatgaagatg

SEQ ID NO: 129 - DNA, 24 bp, Artificial Sequence
  Description of Artificial SequenceArtificially Synthesized DNA Sequence
taagtgtatttagtagaatgaagt

SEQ ID NO: 130 - DNA, 24 bp, Artificial Sequence
  Description of Artificial SequenceArtificially Synthesized DNA Sequence
tatgttagatttgttgagattgat

SEQ ID NO: 131 - DNA, 24 bp, Artificial Sequence
  Description of Artificial SequenceArtificially Synthesized DNA Sequence
agtttgtatgaagagatagtattt

SEQ ID NO: 132 - DNA, 24 bp, Artificial Sequence
  Description of Artificial SequenceArtificially Synthesized DNA Sequence
gagaaatgttatgtatttagtagt

SEQ ID NO: 133 - DNA, 24 bp, Artificial Sequence
  Description of Artificial SequenceArtificially Synthesized DNA Sequence
tatgtgagaatgtgtttgatttaa

SEQ ID NO: 134 - DNA, 24 bp, Artificial Sequence
  Description of Artificial SequenceArtificially Synthesized DNA Sequence
gtatgtttgtttatagaatgtatg

SEQ ID NO: 135 - DNA, 24 bp, Artificial Sequence
  Description of Artificial SequenceArtificially Synthesized DNA Sequence
gagtatatagaagaaagaaatttg

SEQ ID NO: 136 - DNA, 24 bp, Artificial Sequence
  Description of Artificial SequenceArtificially Synthesized DNA Sequence
atgagtgaagtaaatgtagttatt

SEQ ID NO: 137 - DNA, 24 bp, Artificial Sequence
  Description of Artificial SequenceArtificially Synthesized DNA Sequence
ttaagaagtgagttattgtgatat

SEQ ID NO: 138 - DNA, 24 bp, Artificial Sequence
  Description of Artificial SequenceArtificially Synthesized DNA Sequence
atgaaatgagaatattgttgtttg

SEQ ID NO: 139 - DNA, 24 bp, Artificial Sequence
  Description of Artificial SequenceArtificially Synthesized DNA Sequence
gattaatgattatgtgaattgatg

SEQ ID NO: 140 - DNA, 24 bp, Artificial Sequence
  Description of Artificial SequenceArtificially Synthesized DNA Sequence
gaaatgttaaagatatgaaagtag

SEQ ID NO: 141 - DNA, 24 bp, Artificial Sequence
  Description of Artificial SequenceArtificially Synthesized DNA Sequence
tattgttgatttgatattagtgtg

SEQ ID NO: 142 - DNA, 24 bp, Artificial Sequence
  Description of Artificial SequenceArtificially Synthesized DNA Sequence
tttatgtttgtgtatgtaagtagt

SEQ ID NO: 143 - DNA, 24 bp, Artificial Sequence
  Description of Artificial SequenceArtificially Synthesized DNA Sequence
aattgaaagaattgtgtgaattga

SEQ ID NO: 144 - DNA, 24 bp, Artificial Sequence
  Description of Artificial SequenceArtificially Synthesized DNA Sequence
tgagtttgaatttgtttgagtaat

SEQ ID NO: 145 - DNA, 24 bp, Artificial Sequence
  Description of Artificial SequenceArtificially Synthesized DNA Sequence
gatgtataatgatgtgtgtaaatt

SEQ ID NO: 146 - DNA, 24 bp, Artificial Sequence
  Description of Artificial SequenceArtificially Synthesized DNA Sequence
atgtgagagaagaatttgtttatt

SEQ ID NO: 147 - DNA, 24 bp, Artificial Sequence
  Description of Artificial SequenceArtificially Synthesized DNA Sequence
gtgataaagtattgttgatagaaa

SEQ ID NO: 148 - DNA, 24 bp, Artificial Sequence
  Description of Artificial SequenceArtificially Synthesized DNA Sequence
gaagtagaatagaaagttaataga

SEQ ID NO: 149 - DNA, 24 bp, Artificial Sequence
  Description of Artificial SequenceArtificially Synthesized DNA Sequence
ttgtgtagttaagagttgtttaat

SEQ ID NO: 150 - DNA, 24 bp, Artificial Sequence
  Description of Artificial SequenceArtificially Synthesized DNA Sequence
tagtagtaagttgttagaatagtt

SEQ ID NO: 151 - DNA, 24 bp, Artificial Sequence
  Description of Artificial SequenceArtificially Synthesized DNA Sequence
aatttgaagtataatgaatgtgtg

SEQ ID NO: 152 - DNA, 24 bp, Artificial Sequence
  Description of Artificial SequenceArtificially Synthesized DNA Sequence
tagaaattgtagtatttgagagaa

SEQ ID NO: 153 - DNA, 24 bp, Artificial Sequence
  Description of Artificial SequenceArtificially Synthesized DNA Sequence
tgtatatgttaatgagatgttgta

SEQ ID NO: 154 - DNA, 24 bp, Artificial Sequence
  Description of Artificial SequenceArtificially Synthesized DNA Sequence
tatttgataagagaatgaagaagt

SEQ ID NO: 155 - DNA, 24 bp, Artificial Sequence
  Description of Artificial SequenceArtificially Synthesized DNA Sequence
ttgaatagtgtaatgaatatgatg

SEQ ID NO: 156 - DNA, 24 bp, Artificial Sequence
  Description of Artificial SequenceArtificially Synthesized DNA Sequence
gtagtttgtgaatagaattagttt

SEQ ID NO: 157 - DNA, 24 bp, Artificial Sequence
  Description of Artificial SequenceArtificially Synthesized DNA Sequence
aaagatgattgtaatttgtgtgaa

SEQ ID NO: 158 - DNA, 24 bp, Artificial Sequence
  Description of Artificial SequenceArtificially Synthesized DNA Sequence
gaagattgttgagttaatagataa

SEQ ID NO: 159 - DNA, 24 bp, Artificial Sequence
  Description of Artificial SequenceArtificially Synthesized DNA Sequence
agattatgtagtgatgtaaatgtt

SEQ ID NO: 160 - DNA, 24 bp, Artificial Sequence
  Description of Artificial SequenceArtificially Synthesized DNA Sequence
gaatttagatgtagatatgaatgt

SEQ ID NO: 161 - DNA, 24 bp, Artificial Sequence
  Description of Artificial SequenceArtificially Synthesized DNA Sequence
gatagaagtgtattaagtaagtta

SEQ ID NO: 162 - DNA, 24 bp, Artificial Sequence
  Description of Artificial SequenceArtificially Synthesized DNA Sequence
tatgaattatgagaagaatagagt

SEQ ID NO: 163 - DNA, 24 bp, Artificial Sequence
  Description of Artificial SequenceArtificially Synthesized DNA Sequence
tttgttatgaagtgatttgtttgt

SEQ ID NO: 164 - DNA, 24 bp, Artificial Sequence
  Description of Artificial SequenceArtificially Synthesized DNA Sequence
gtaaagattgtgttatatgaaatg

SEQ ID NO: 165 - DNA, 24 bp, Artificial Sequence
  Description of Artificial SequenceArtificially Synthesized DNA Sequence
ttgtgatagtagttagatatttgt

SEQ ID NO: 166 - DNA, 24 bp, Artificial Sequence
  Description of Artificial SequenceArtificially Synthesized DNA Sequence
gaattaagataaagaagagaagta

SEQ ID NO: 167 - DNA, 24 bp, Artificial Sequence
  Description of Artificial SequenceArtificially Synthesized DNA Sequence
gattgtagaatgaatttgtagtat

SEQ ID NO: 168 - DNA, 24 bp, Artificial Sequence
  Description of Artificial SequenceArtificially Synthesized DNA Sequence
aaataagagagagaatgatttagt

SEQ ID NO: 169 - DNA, 24 bp, Artificial Sequence
  Description of Artificial SequenceArtificially Synthesized DNA Sequence
aattatgtgaatagattgttgaag

SEQ ID NO: 170 - DNA, 24 bp, Artificial Sequence
  Description of Artificial SequenceArtificially Synthesized DNA Sequence
ttaagatttatgtgatagtagagt

SEQ ID NO: 171 - DNA, 24 bp, Artificial Sequence
  Description of Artificial SequenceArtificially Synthesized DNA Sequence
ttaaagatagtgtttgttgtgtta

SEQ ID NO: 172 - DNA, 24 bp, Artificial Sequence
  Description of Artificial SequenceArtificially Synthesized DNA Sequence
tattgatttatgaagagtatagtg

SEQ ID NO: 173 - DNA, 24 bp, Artificial Sequence
  Description of Artificial SequenceArtificially Synthesized DNA Sequence
aaatttgatgagtagtttaagaga

SEQ ID NO: 174 - DNA, 24 bp, Artificial Sequence
  Description of Artificial SequenceArtificially Synthesized DNA Sequence
ataaagttgtttgatgtttgaatg

SEQ ID NO: 175 - DNA, 24 bp, Artificial Sequence
  Description of Artificial SequenceArtificially Synthesized DNA Sequence
gattgtgatgaataatgttattga

SEQ ID NO: 176 - DNA, 24 bp, Artificial Sequence
  Description of Artificial SequenceArtificially Synthesized DNA Sequence
gatgaagaaatatgatatgaatag

SEQ ID NO: 177 - DNA, 24 bp, Artificial Sequence
  Description of Artificial SequenceArtificially Synthesized DNA Sequence
ttaaagttattgaagtgaagttga

SEQ ID NO: 178 - DNA, 24 bp, Artificial Sequence
  Description of Artificial SequenceArtificially Synthesized DNA Sequence
ttgtaagaaatagagatttgtgtt

SEQ ID NO: 179 - DNA, 24 bp, Artificial Sequence
  Description of Artificial SequenceArtificially Synthesized DNA Sequence
gagattgagtttaagtattagatt

SEQ ID NO: 180 - DNA, 24 bp, Artificial Sequence
  Description of Artificial SequenceArtificially Synthesized DNA Sequence
agtgataatagaatgataaatgtg

SEQ ID NO: 181 - DNA, 24 bp, Artificial Sequence
  Description of Artificial SequenceArtificially Synthesized DNA Sequence
gataatagtgaatttgagttgtat

SEQ ID NO: 182 - DNA, 24 bp, Artificial Sequence
  Description of Artificial SequenceArtificially Synthesized DNA Sequence
agatatttgtagtagaaagtatgt

SEQ ID NO: 183 - DNA, 24 bp, Artificial Sequence
  Description of Artificial SequenceArtificially Synthesized DNA Sequence
gttatgaatgttgaatttgaatgt

SEQ ID NO: 184 - DNA, 24 bp, Artificial Sequence
  Description of Artificial SequenceArtificially Synthesized DNA Sequence
atgaaagatttagttgtgagatat

SEQ ID NO: 185 - DNA, 24 bp, Artificial Sequence
  Description of Artificial SequenceArtificially Synthesized DNA Sequence
aaatagagaagttatgatgtgata

SEQ ID NO: 186 - DNA, 24 bp, Artificial Sequence
  Description of Artificial SequenceArtificially Synthesized DNA Sequence
ttagtgagaaatgtttaatgtgat

SEQ ID NO: 187 - DNA, 24 bp, Artificial Sequence
  Description of Artificial SequenceArtificially Synthesized DNA Sequence
tgaagaatatgtgaaattagtttg

SEQ ID NO: 188 - DNA, 24 bp, Artificial Sequence
  Description of Artificial SequenceArtificially Synthesized DNA Sequence
gtttgatagtttaatgagtattga

SEQ ID NO: 189 - DNA, 24 bp, Artificial Sequence
  Description of Artificial SequenceArtificially Synthesized DNA Sequence
gttgtaagtaatgataaagtatga

SEQ ID NO: 190 - DNA, 24 bp, Artificial Sequence
  Description of Artificial SequenceArtificially Synthesized DNA Sequence
taagagtagtaattgttgtttaga

SEQ ID NO: 191 - DNA, 24 bp, Artificial Sequence
  Description of Artificial SequenceArtificially Synthesized DNA Sequence
tttgagagagtatgtatgattatt

SEQ ID NO: 192 - DNA, 24 bp, Artificial Sequence
  Description of Artificial SequenceArtificially Synthesized DNA Sequence
attgattgtgaattagatagaaga

SEQ ID NO: 193 - DNA, 24 bp, Artificial Sequence
  Description of Artificial SequenceArtificially Synthesized DNA Sequence
gattagtatttagtagtaatagag

SEQ ID NO: 194 - DNA, 24 bp, Artificial Sequence
  Description of Artificial SequenceArtificially Synthesized DNA Sequence
tatgtattagagatattgaaagtg

SEQ ID NO: 195 - DNA, 24 bp, Artificial Sequence
  Description of Artificial SequenceArtificially Synthesized DNA Sequence
tatgtgaaagtaatgataaatgag

SEQ ID NO: 196 - DNA, 24 bp, Artificial Sequence
  Description of Artificial SequenceArtificially Synthesized DNA Sequence
gtaattagtaatgatttgaatgag

SEQ ID NO: 197 - DNA, 24 bp, Artificial Sequence
  Description of Artificial SequenceArtificially Synthesized DNA Sequence
gtttattgtaaagatgtaagtgaa

SEQ ID NO: 198 - DNA, 24 bp, Artificial Sequence
  Description of Artificial SequenceArtificially Synthesized DNA Sequence
tagtagaattgttgttaaagaatg

SEQ ID NO: 199 - DNA, 24 bp, Artificial Sequence
  Description of Artificial SequenceArtificially Synthesized DNA Sequence
tattgttagttatgtagtgtgtaa

SEQ ID NO: 200 - DNA, 24 bp, Artificial Sequence
  Description of Artificial SequenceArtificially Synthesized DNA Sequence
gagtgaaagttatatgaaagtata

SEQ ID NO: 201 - DNA, 24 bp, Artificial Sequence
  Description of Artificial SequenceArtificially Synthesized DNA Sequence
atatagaagttgatgagtttatga

SEQ ID NO: 202 - DNA, 24 bp, Artificial Sequence
  Description of Artificial SequenceArtificially Synthesized DNA Sequence
tttagaagtaagaataagtgagta

SEQ ID NO: 203 - DNA, 24 bp, Artificial Sequence
  Description of Artificial SequenceArtificially Synthesized DNA Sequence
tgtgtataagatatttgtaagaag

SEQ ID NO: 204 - DNA, 24 bp, Artificial Sequence
  Description of Artificial SequenceArtificially Synthesized DNA Sequence
tagaagagttgtattgttataagt

SEQ ID NO: 205 - DNA, 24 bp, Artificial Sequence
  Description of Artificial SequenceArtificially Synthesized DNA Sequence
gtgttattagtttaagttagagta

SEQ ID NO: 206 - DNA, 24 bp, Artificial Sequence
  Description of Artificial SequenceArtificially Synthesized DNA Sequence
aatatagtgatgtgaaattgaatg

SEQ ID NO: 207 - DNA, 24 bp, Artificial Sequence
  Description of Artificial SequenceArtificially Synthesized DNA Sequence
ttagagaatagagtgattatagtt

SEQ ID NO: 208 - DNA, 24 bp, Artificial Sequence
  Description of Artificial SequenceArtificially Synthesized DNA Sequence
gaagtgagttaatgatttgtaaat

SEQ ID NO: 209 - DNA, 24 bp, Artificial Sequence
  Description of Artificial SequenceArtificially Synthesized DNA Sequence
aatgtaaagtaaagaaagtgatga

SEQ ID NO: 210 - DNA, 24 bp, Artificial Sequence
  Description of Artificial SequenceArtificially Synthesized DNA Sequence
gttagttatgatgaatattgtgta

SEQ ID NO: 211 - DNA, 24 bp, Artificial Sequence
  Description of Artificial SequenceArtificially Synthesized DNA Sequence
aaatgagttagagtagaattatgt

SEQ ID NO: 212 - DNA, 24 bp, Artificial Sequence
  Description of Artificial SequenceArtificially Synthesized DNA Sequence
gatatagaagattagttagtgata

SEQ ID NO: 213 - DNA, 24 bp, Artificial Sequence
  Description of Artificial SequenceArtificially Synthesized DNA Sequence
atagtttgttgagatttatgagta

SEQ ID NO: 214 - DNA, 24 bp, Artificial Sequence
  Description of Artificial SequenceArtificially Synthesized DNA Sequence
tagaatagttagtagtaagagtat

SEQ ID NO: 215 - DNA, 24 bp, Artificial Sequence
  Description of Artificial SequenceArtificially Synthesized DNA Sequence
gaatttgtattgtgaagtttagta

SEQ ID NO: 216 - DNA, 24 bp, Artificial Sequence
  Description of Artificial SequenceArtificially Synthesized DNA Sequence
gtagtaagaagagaattagattaa

SEQ ID NO: 217 - DNA, 24 bp, Artificial Sequence
  Description of Artificial SequenceArtificially Synthesized DNA Sequence
aatgtgttatgtatgtaaatagtg

SEQ ID NO: 218 - DNA, 24 bp, Artificial Sequence
  Description of Artificial SequenceArtificially Synthesized DNA Sequence
gaattagttagagtaaattgtttg

SEQ ID NO: 219 - DNA, 24 bp, Artificial Sequence
  Description of Artificial SequenceArtificially Synthesized DNA Sequence
gaaattgaagatagtaagaaatga

SEQ ID NO: 220 - DNA, 24 bp, Artificial Sequence
  Description of Artificial SequenceArtificially Synthesized DNA Sequence
gtgtattatgtgatttatgataga

SEQ ID NO: 221 - DNA, 24 bp, Artificial Sequence
  Description of Artificial SequenceArtificially Synthesized DNA Sequence
tattatgagaaagttgaatagtag

SEQ ID NO: 222 - DNA, 24 bp, Artificial Sequence
  Description of Artificial SequenceArtificially Synthesized DNA Sequence
tatgtattgtattgagtagatgaa

SEQ ID NO: 223 - DNA, 24 bp, Artificial Sequence
  Description of Artificial SequenceArtificially Synthesized DNA Sequence
gtgattgaatagtagattgtttaa

SEQ ID NO: 224 - DNA, 24 bp, Artificial Sequence
  Description of Artificial SequenceArtificially Synthesized DNA Sequence
agtaagttgtttgattgaaatttg

SEQ ID NO: 225 - DNA, 24 bp, Artificial Sequence
  Description of Artificial SequenceArtificially Synthesized DNA Sequence
gaagtttgatttaagtttaagaag

SEQ ID NO: 226 - DNA, 24 bp, Artificial Sequence
  Description of Artificial SequenceArtificially Synthesized DNA Sequence
gagaagataaatgatattgttatg

SEQ ID NO: 227 - DNA, 24 bp, Artificial Sequence
  Description of Artificial SequenceArtificially Synthesized DNA Sequence
atgatgagttgttaatagttagtt

SEQ ID NO: 228 - DNA, 24 bp, Artificial Sequence
  Description of Artificial SequenceArtificially Synthesized DNA Sequence
tatgatatttgaagagtgttaaga

SEQ ID NO: 229 - DNA, 24 bp, Artificial Sequence
  Description of Artificial SequenceArtificially Synthesized DNA Sequence
gagatgattaaagtgatttatgaa

SEQ ID NO: 230 - DNA, 24 bp, Artificial Sequence
  Description of Artificial SequenceArtificially Synthesized DNA Sequence
atagttaagagtgatgagaataaa

SEQ ID NO: 231 - DNA, 24 bp, Artificial Sequence
  Description of Artificial SequenceArtificially Synthesized DNA Sequence
tttattgttagataaagagttgag

SEQ ID NO: 232 - DNA, 24 bp, Artificial Sequence
  Description of Artificial SequenceArtificially Synthesized DNA Sequence
agaatattgatagttgaagttgaa

SEQ ID NO: 233 - DNA, 24 bp, Artificial Sequence
  Description of Artificial SequenceArtificially Synthesized DNA Sequence
tagtgtaaagtgtagattgtaaat

SEQ ID NO: 234 - DNA, 24 bp, Artificial Sequence
  Description of Artificial SequenceArtificially Synthesized DNA Sequence
agtagtgatatgatttgaatattg

SEQ ID NO: 235 - DNA, 24 bp, Artificial Sequence
  Description of Artificial SequenceArtificially Synthesized DNA Sequence
tgtattgaattagaatagtgagaa

SEQ ID NO: 236 - DNA, 24 bp, Artificial Sequence
  Description of Artificial SequenceArtificially Synthesized DNA Sequence
tgatatgagatagaagtttaatgt

SEQ ID NO: 237 - DNA, 24 bp, Artificial Sequence
  Description of Artificial SequenceArtificially Synthesized DNA Sequence
gaagaagtaagtataaagtaaatg

SEQ ID NO: 238 - DNA, 24 bp, Artificial Sequence
  Description of Artificial SequenceArtificially Synthesized DNA Sequence
tttaagtgtgataagaaagataga

SEQ ID NO: 239 - DNA, 24 bp, Artificial Sequence
  Description of Artificial SequenceArtificially Synthesized DNA Sequence
tattgttgaatgtgtttaaagaga

SEQ ID NO: 240 - DNA, 24 bp, Artificial Sequence
  Description of Artificial SequenceArtificially Synthesized DNA Sequence
gaataatgatgagatgattattga

SEQ ID NO: 241 - DNA, 24 bp, Artificial Sequence
  Description of Artificial SequenceArtificially Synthesized DNA Sequence
tagagaaagagagaattgtattaa

SEQ ID NO: 242 - DNA, 24 bp, Artificial Sequence
  Description of Artificial SequenceArtificially Synthesized DNA Sequence
atgtataatgagatatgtttgtga

SEQ ID NO: 243 - DNA, 24 bp, Artificial Sequence
  Description of Artificial SequenceArtificially Synthesized DNA Sequence
aatagataagattgattgtgtttg

SEQ ID NO: 244 - DNA, 24 bp, Artificial Sequence
  Description of Artificial SequenceArtificially Synthesized DNA Sequence
tttgatgataatagaagagaatga

SEQ ID NO: 245 - DNA, 24 bp, Artificial Sequence
  Description of Artificial SequenceArtificially Synthesized DNA Sequence
agatgaataagttgtgaatgttta

SEQ ID NO: 246 - DNA, 24 bp, Artificial Sequence
  Description of Artificial SequenceArtificially Synthesized DNA Sequence
agatgaaagaaagtgtagaatatt

SEQ ID NO: 247 - DNA, 24 bp, Artificial Sequence
  Description of Artificial SequenceArtificially Synthesized DNA Sequence
tgttaaatgtatgtagtaattgag

SEQ ID NO: 248 - DNA, 24 bp, Artificial Sequence
  Description of Artificial SequenceArtificially Synthesized DNA Sequence
tagtagtgtgaagttatttgttat

SEQ ID NO: 249 - DNA, 24 bp, Artificial Sequence
  Description of Artificial SequenceArtificially Synthesized DNA Sequence
agtgaatgtttgtaaagagtttaa

SEQ ID NO: 250 - DNA, 24 bp, Artificial Sequence
  Description of Artificial SequenceArtificially Synthesized DNA Sequence
gataaatgagaattgagtaattgt

SEQ ID NO: 251 - DNA, 24 bp, Artificial Sequence
  Description of Artificial SequenceArtificially Synthesized DNA Sequence
tgatgagaaattgtttaagtgttt

SEQ ID NO: 252 - DNA, 24 bp, Artificial Sequence
  Description of Artificial SequenceArtificially Synthesized DNA Sequence
aaataagtagtgtgagtaatagta

SEQ ID NO: 253 - DNA, 24 bp, Artificial Sequence
  Description of Artificial SequenceArtificially Synthesized DNA Sequence
tatgaaatatgtgatagtaagaga

SEQ ID NO: 254 - DNA, 24 bp, Artificial Sequence
  Description of Artificial SequenceArtificially Synthesized DNA Sequence
attgtaagagtgattatagatgat

SEQ ID NO: 255 - DNA, 24 bp, Artificial Sequence
  Description of Artificial SequenceArtificially Synthesized DNA Sequence
agagtaagaatgaaagagataata

SEQ ID NO: 256 - DNA, 24 bp, Artificial Sequence
  Description of Artificial SequenceArtificially Synthesized DNA Sequence
taagtaagtagatgttaaagagat

SEQ ID NO: 257 - DNA, 24 bp, Artificial Sequence
  Description of Artificial SequenceArtificially Synthesized DNA Sequence
aaatagaaagaattgtagagtagt

SEQ ID NO: 258 - DNA, 24 bp, Artificial Sequence
  Description of Artificial SequenceArtificially Synthesized DNA Sequence
atagatttaagtgaagagagttat

SEQ ID NO: 259 - DNA, 24 bp, Artificial Sequence
  Description of Artificial SequenceArtificially Synthesized DNA Sequence
gaatgtttgtaaatgtatagatag

SEQ ID NO: 260 - DNA, 24 bp, Artificial Sequence
  Description of Artificial SequenceArtificially Synthesized DNA Sequence
aaatagaatgagtagtgaaatatg

SEQ ID NO: 261 - DNA, 24 bp, Artificial Sequence
  Description of Artificial SequenceArtificially Synthesized DNA Sequence
ttgaattatgtagagaaagtaaag

SEQ ID NO: 262 - DNA, 24 bp, Artificial Sequence
  Description of Artificial SequenceArtificially Synthesized DNA Sequence
tagtaaattgagagtagttgaatt

SEQ ID NO: 263 - DNA, 24 bp, Artificial Sequence
  Description of Artificial SequenceArtificially Synthesized DNA Sequence
tgtaaagtgtttatagtgtgtaat

SEQ ID NO: 264 - DNA, 24 bp, Artificial Sequence
  Description of Artificial SequenceArtificially Synthesized DNA Sequence
atatgatttgagatgagaatgtaa

SEQ ID NO: 265 - DNA, 24 bp, Artificial Sequence
  Description of Artificial SequenceArtificially Synthesized DNA Sequence
aatattgatatgtgttgtgaagta

SEQ ID NO: 266 - DNA, 24 bp, Artificial Sequence
  Description of Artificial SequenceArtificially Synthesized DNA Sequence
agtgagattatgagtattgattta

SEQ ID NO: 267 - DNA, 24 bp, Artificial Sequence
  Description of Artificial SequenceArtificially Synthesized DNA Sequence
ttgtatttagatagtgagattatg

SEQ ID NO: 268 - DNA, 24 bp, Artificial Sequence
  Description of Artificial SequenceArtificially Synthesized DNA Sequence
atagaaatgaaagatagatagaag

SEQ ID NO: 269 - DNA, 24 bp, Artificial Sequence
  Description of Artificial SequenceArtificially Synthesized DNA Sequence
gattgtatatgtaaagtagtttag

SEQ ID NO: 270 - DNA, 24 bp, Artificial Sequence
  Description of Artificial SequenceArtificially Synthesized DNA Sequence
tatgaatgttattgtgtgttgatt

SEQ ID NO: 271 - DNA, 24 bp, Artificial Sequence
  Description of Artificial SequenceArtificially Synthesized DNA Sequence
gatattagtagagtaagtatattg

SEQ ID NO: 272 - DNA, 24 bp, Artificial Sequence
  Description of Artificial SequenceArtificially Synthesized DNA Sequence
tgagatgaatttgtgttatgatat

SEQ ID NO: 273 - DNA, 24 bp, Artificial Sequence
  Description of Artificial SequenceArtificially Synthesized DNA Sequence
tatgaatgaagtaaagagatgtaa

SEQ ID NO: 274 - DNA, 24 bp, Artificial Sequence
  Description of Artificial SequenceArtificially Synthesized DNA Sequence
gagtgaatttgttgtaatttgttt

SEQ ID NO: 275 - DNA, 24 bp, Artificial Sequence
  Description of Artificial SequenceArtificially Synthesized DNA Sequence
agaaattgtagagttaattgtgta

SEQ ID NO: 276 - DNA, 24 bp, Artificial Sequence
  Description of Artificial SequenceArtificially Synthesized DNA Sequence
gtgttaatgaaagttgtgaataat

SEQ ID NO: 277 - DNA, 24 bp, Artificial Sequence
  Description of Artificial SequenceArtificially Synthesized DNA Sequence
tgtgatttgttaagaagattaatg

SEQ ID NO: 278 - DNA, 24 bp, Artificial Sequence
  Description of Artificial SequenceArtificially Synthesized DNA Sequence
agtagtattgtaaagtataaagag

SEQ ID NO: 279 - DNA, 24 bp, Artificial Sequence
  Description of Artificial SequenceArtificially Synthesized DNA Sequence
tgattgttgtatagttattgtgta

SEQ ID NO: 280 - DNA, 24 bp, Artificial Sequence
  Description of Artificial SequenceArtificially Synthesized DNA Sequence
gattgtagtttaatgttaagaatg

SEQ ID NO: 281 - DNA, 24 bp, Artificial Sequence
  Description of Artificial SequenceArtificially Synthesized DNA Sequence
atgaaataagaaattgagtagaga

SEQ ID NO: 282 - DNA, 24 bp, Artificial Sequence
  Description of Artificial SequenceArtificially Synthesized DNA Sequence
tatgatgatatttgttgtatgtgt

SEQ ID NO: 283 - DNA, 24 bp, Artificial Sequence
  Description of Artificial SequenceArtificially Synthesized DNA Sequence
tttagagtttgattagtatgtttg

SEQ ID NO: 284 - DNA, 24 bp, Artificial Sequence
  Description of Artificial SequenceArtificially Synthesized DNA Sequence
aataagagattgtgatgagaaata

SEQ ID NO: 285 - DNA, 24 bp, Artificial Sequence
  Description of Artificial SequenceArtificially Synthesized DNA Sequence
aatgaatagaatagagaatgtaga

SEQ ID NO: 286 - DNA, 24 bp, Artificial Sequence
  Description of Artificial SequenceArtificially Synthesized DNA Sequence
gtagtagtaatttgaatgtttgaa

SEQ ID NO: 287 - DNA, 24 bp, Artificial Sequence
  Description of Artificial SequenceArtificially Synthesized DNA Sequence
agtgagtaattgattgattgttaa

SEQ ID NO: 288 - DNA, 24 bp, Artificial Sequence
  Description of Artificial SequenceArtificially Synthesized DNA Sequence
gaataatgtttagtgtgtttgaaa

SEQ ID NO: 289 - DNA, 24 bp, Artificial Sequence
  Description of Artificial SequenceArtificially Synthesized DNA Sequence
atatgaaagtagagaaagtgttat

SEQ ID NO: 290 - DNA, 24 bp, Artificial Sequence
  Description of Artificial SequenceArtificially Synthesized DNA Sequence
tgagttattgtatttagtttgaag

SEQ ID NO: 291 - DNA, 24 bp, Artificial Sequence
  Description of Artificial SequenceArtificially Synthesized DNA Sequence
tagttgagtttaaagttgaaagaa

SEQ ID NO: 292 - DNA, 24 bp, Artificial Sequence
  Description of Artificial SequenceArtificially Synthesized DNA Sequence
taaagagtgatgtaaatagaagtt

SEQ ID NO: 293 - DNA, 24 bp, Artificial Sequence
  Description of Artificial SequenceArtificially Synthesized DNA Sequence
tgtagtgtttagagtaagttatta

SEQ ID NO: 294 - DNA, 24 bp, Artificial Sequence
  Description of Artificial SequenceArtificially Synthesized DNA Sequence
agagattaatgtgttgaaagatta

SEQ ID NO: 295 - DNA, 24 bp, Artificial Sequence
  Description of Artificial SequenceArtificially Synthesized DNA Sequence
gtaataagttgtgaaagaagatta

SEQ ID NO: 296 - DNA, 24 bp, Artificial Sequence
  Description of Artificial SequenceArtificially Synthesized DNA Sequence
gagatgttatagataatgaaagaa

SEQ ID NO: 297 - DNA, 24 bp, Artificial Sequence
  Description of Artificial SequenceArtificially Synthesized DNA Sequence
tttagttgattgttgaatagagta

SEQ ID NO: 298 - DNA, 24 bp, Artificial Sequence
  Description of Artificial SequenceArtificially Synthesized DNA Sequence
attattgaaagtagatgttagatg

SEQ ID NO: 299 - DNA, 24 bp, Artificial Sequence
  Description of Artificial SequenceArtificially Synthesized DNA Sequence
tttatgtgtgattgagtgtttaat

SEQ ID NO: 300 - DNA, 24 bp, Artificial Sequence
  Description of Artificial SequenceArtificially Synthesized DNA Sequence
tatttagttagatagatagagagt

SEQ ID NO: 301 - DNA, 24 bp, Artificial Sequence
  Description of Artificial SequenceArtificially Synthesized DNA Sequence
atgtgtttatgtgaaagatttgta

SEQ ID NO: 302 - DNA, 24 bp, Artificial Sequence
  Description of Artificial SequenceArtificially Synthesized DNA Sequence
atagtaattagaagagaagaatgt

SEQ ID NO: 303 - DNA, 24 bp, Artificial Sequence
  Description of Artificial SequenceArtificially Synthesized DNA Sequence
tatgagtgattagaattgtatttg

SEQ ID NO: 304 - DNA, 24 bp, Artificial Sequence
  Description of Artificial SequenceArtificially Synthesized DNA Sequence
ttaatgtattgtttaaagagtgtg

SEQ ID NO: 305 - DNA, 24 bp, Artificial Sequence
  Description of Artificial SequenceArtificially Synthesized DNA Sequence
atagagaattaagaattgtttgag

SEQ ID NO: 306 - DNA, 24 bp, Artificial Sequence
  Description of Artificial SequenceArtificially Synthesized DNA Sequence
gttataagtagaaatgtatagaag

SEQ ID NO: 307 - DNA, 24 bp, Artificial Sequence
  Description of Artificial SequenceArtificially Synthesized DNA Sequence
agtaattagtttgaaatgtgtagt

SEQ ID NO: 308 - DNA, 24 bp, Artificial Sequence
  Description of Artificial SequenceArtificially Synthesized DNA Sequence
gaaagattatgattgtaaagtgat

SEQ ID NO: 309 - DNA, 24 bp, Artificial Sequence
  Description of Artificial SequenceArtificially Synthesized DNA Sequence
gtaagattagaagttaatgaagaa

SEQ ID NO: 310 - DNA, 24 bp, Artificial Sequence
  Description of Artificial SequenceArtificially Synthesized DNA Sequence
gagaatgttgaataagaagtaatt

SEQ ID NO: 311 - DNA, 24 bp, Artificial Sequence
  Description of Artificial SequenceArtificially Synthesized DNA Sequence
ttaagagtgtttgaatagtgttta

SEQ ID NO: 312 - DNA, 24 bp, Artificial Sequence
  Description of Artificial SequenceArtificially Synthesized DNA Sequence
ataaagaaagagtatgagattatg

SEQ ID NO: 313 - DNA, 24 bp, Artificial Sequence
  Description of Artificial SequenceArtificially Synthesized DNA Sequence
agttattgattgaagatgagaaat

SEQ ID NO: 314 - DNA, 24 bp, Artificial Sequence
  Description of Artificial SequenceArtificially Synthesized DNA Sequence
gtttgtgtttgtataagttgttaa

SEQ ID NO: 315 - DNA, 24 bp, Artificial Sequence
  Description of Artificial SequenceArtificially Synthesized DNA Sequence
ttgtatgtgagtttagattaatga

SEQ ID NO: 316 - DNA, 24 bp, Artificial Sequence
  Description of Artificial SequenceArtificially Synthesized DNA Sequence
tagttaaagtatagttgtttgagt

SEQ ID NO: 317 - DNA, 24 bp, Artificial Sequence
  Description of Artificial SequenceArtificially Synthesized DNA Sequence
aaatttgtgttgagatttgtatag

SEQ ID NO: 318 - DNA, 24 bp, Artificial Sequence
  Description of Artificial SequenceArtificially Synthesized DNA Sequence
tattagtgttatgataaagagaag

SEQ ID NO: 319 - DNA, 24 bp, Artificial Sequence
  Description of Artificial SequenceArtificially Synthesized DNA Sequence
tataagaagtaatttgagaagagt

SEQ ID NO: 320 - DNA, 24 bp, Artificial Sequence
  Description of Artificial SequenceArtificially Synthesized DNA Sequence
taagttgagatgtttgtttgataa

SEQ ID NO: 321 - DNA, 24 bp, Artificial Sequence
  Description of Artificial SequenceArtificially Synthesized DNA Sequence
gtgtagatttatgaattgagtaat

SEQ ID NO: 322 - DNA, 24 bp, Artificial Sequence
  Description of Artificial SequenceArtificially Synthesized DNA Sequence
tatagagaagtgtttagttgtata

SEQ ID NO: 323 - DNA, 24 bp, Artificial Sequence
  Description of Artificial SequenceArtificially Synthesized DNA Sequence
ataaagaagaatagttgttgtgta

SEQ ID NO: 324 - DNA, 24 bp, Artificial Sequence
  Description of Artificial SequenceArtificially Synthesized DNA Sequence
agattgaaatagattagaaagttg

SEQ ID NO: 325 - DNA, 24 bp, Artificial Sequence
  Description of Artificial SequenceArtificially Synthesized DNA Sequence
gttgttataagaaatagtttgttg

SEQ ID NO: 326 - DNA, 24 bp, Artificial Sequence
  Description of Artificial SequenceArtificially Synthesized DNA Sequence
agaaatagagtaagagtgtttaaa

SEQ ID NO: 327 - DNA, 24 bp, Artificial Sequence
  Description of Artificial SequenceArtificially Synthesized DNA Sequence
agagatagtagtaaatagttattg

SEQ ID NO: 328 - DNA, 24 bp, Artificial Sequence
  Description of Artificial SequenceArtificially Synthesized DNA Sequence
aaatgattgtgtaagttatgtatg

SEQ ID NO: 329 - DNA, 24 bp, Artificial Sequence
  Description of Artificial SequenceArtificially Synthesized DNA Sequence
aagaagtaagagagaaatttgaat

SEQ ID NO: 330 - DNA, 24 bp, Artificial Sequence
  Description of Artificial SequenceArtificially Synthesized DNA Sequence
gtgtgtatttagttgataattgat

SEQ ID NO: 331 - DNA, 24 bp, Artificial Sequence
  Description of Artificial SequenceArtificially Synthesized DNA Sequence
attgttgttgttgagaaatgtatt

SEQ ID NO: 332 - DNA, 24 bp, Artificial Sequence
  Description of Artificial SequenceArtificially Synthesized DNA Sequence
agataagttaaagtaaagagaatg

SEQ ID NO: 333 - DNA, 24 bp, Artificial Sequence
  Description of Artificial SequenceArtificially Synthesized DNA Sequence
tagttgaagttagtttaagtgtta

SEQ ID NO: 334 - DNA, 24 bp, Artificial Sequence
  Description of Artificial SequenceArtificially Synthesized DNA Sequence
agtaagaatgtaatatgatgatag

SEQ ID NO: 335 - DNA, 24 bp, Artificial Sequence
  Description of Artificial SequenceArtificially Synthesized DNA Sequence
atgagattgaaagatttatgaatg

SEQ ID NO: 336 - DNA, 24 bp, Artificial Sequence
  Description of Artificial SequenceArtificially Synthesized DNA Sequence
tgattgaattagagagaatgtata

SEQ ID NO: 337 - DNA, 24 bp, Artificial Sequence
  Description of Artificial SequenceArtificially Synthesized DNA Sequence
agttagtaagagaatatagtgaat

SEQ ID NO: 338 - DNA, 24 bp, Artificial Sequence
  Description of Artificial SequenceArtificially Synthesized DNA Sequence
attaagattgtatagttagtgatg

SEQ ID NO: 339 - DNA, 24 bp, Artificial Sequence
  Description of Artificial SequenceArtificially Synthesized DNA Sequence
gagataaagaattgaaatagaaga

SEQ ID NO: 340 - DNA, 24 bp, Artificial Sequence
  Description of Artificial SequenceArtificially Synthesized DNA Sequence
agagtaaatgttaagaaagaagtt

SEQ ID NO: 341 - DNA, 24 bp, Artificial Sequence
  Description of Artificial SequenceArtificially Synthesized DNA Sequence
aaagtttgttatgtgtgaagaatt

SEQ ID NO: 342 - DNA, 24 bp, Artificial Sequence
  Description of Artificial SequenceArtificially Synthesized DNA Sequence
attgtgtttaagaaatatgatgag

SEQ ID NO: 343 - DNA, 24 bp, Artificial Sequence
  Description of Artificial SequenceArtificially Synthesized DNA Sequence
tattgaaatgagatgtatgtagtt

SEQ ID NO: 344 - DNA, 24 bp, Artificial Sequence
  Description of Artificial SequenceArtificially Synthesized DNA Sequence
atttgtgtgatgtttgaaatatga

SEQ ID NO: 345 - DNA, 24 bp, Artificial Sequence
  Description of Artificial SequenceArtificially Synthesized DNA Sequence
taagataatagtgagagaaattga

SEQ ID NO: 346 - DNA, 24 bp, Artificial Sequence
  Description of Artificial SequenceArtificially Synthesized DNA Sequence
atttatgattagtgtaagtgttgt

SEQ ID NO: 347 - DNA, 24 bp, Artificial Sequence
  Description of Artificial SequenceArtificially Synthesized DNA Sequence
gattaagaataaagtgtgaagaat

SEQ ID NO: 348 - DNA, 24 bp, Artificial Sequence
  Description of Artificial SequenceArtificially Synthesized DNA Sequence
gtaattgatgaagagttagtttat

SEQ ID NO: 349 - DNA, 24 bp, Artificial Sequence
  Description of Artificial SequenceArtificially Synthesized DNA Sequence
tgtgttatgttataagaagtgata

SEQ ID NO: 350 - DNA, 24 bp, Artificial Sequence
  Description of Artificial SequenceArtificially Synthesized DNA Sequence
agagaaattgaatttagaaatgtg

SEQ ID NO: 351 - DNA, 24 bp, Artificial Sequence
  Description of Artificial SequenceArtificially Synthesized DNA Sequence
ttattgaatgtgagaaagtatttg

SEQ ID NO: 352 - DNA, 24 bp, Artificial Sequence
  Description of Artificial SequenceArtificially Synthesized DNA Sequence
tgttaatgagaagataatgatagt

SEQ ID NO: 353 - DNA, 24 bp, Artificial Sequence
  Description of Artificial SequenceArtificially Synthesized DNA Sequence
gaaagtatttgttgattattgttg

SEQ ID NO: 354 - DNA, 24 bp, Artificial Sequence
  Description of Artificial SequenceArtificially Synthesized DNA Sequence
tagtttatgtagttaattgttgag

SEQ ID NO: 355 - DNA, 24 bp, Artificial Sequence
  Description of Artificial SequenceArtificially Synthesized DNA Sequence
gttgaaagatagtttgatatgtat

SEQ ID NO: 356 - DNA, 24 bp, Artificial Sequence
  Description of Artificial SequenceArtificially Synthesized DNA Sequence
ttagaagatagattattgagaaag

SEQ ID NO: 357 - DNA, 24 bp, Artificial Sequence
  Description of Artificial SequenceArtificially Synthesized DNA Sequence
aataatgttgtgaaatagatgtga

SEQ ID NO: 358 - DNA, 24 bp, Artificial Sequence
  Description of Artificial SequenceArtificially Synthesized DNA Sequence
agtaagaaagtttagtttagttag

SEQ ID NO: 359 - DNA, 24 bp, Artificial Sequence
  Description of Artificial SequenceArtificially Synthesized DNA Sequence
tagtttaatgagatgtttgatatg

SEQ ID NO: 360 - DNA, 24 bp, Artificial Sequence
  Description of Artificial SequenceArtificially Synthesized DNA Sequence
ttaaagatgttaaagaatgagtga

SEQ ID NO: 361 - DNA, 24 bp, Artificial Sequence
  Description of Artificial SequenceArtificially Synthesized DNA Sequence
aaagtgtgtatatgttagaaagta

SEQ ID NO: 362 - DNA, 24 bp, Artificial Sequence
  Description of Artificial SequenceArtificially Synthesized DNA Sequence
attaagttatgtgtttatgtgttg

SEQ ID NO: 363 - DNA, 24 bp, Artificial Sequence
  Description of Artificial SequenceArtificially Synthesized DNA Sequence
tttgaagaagtgtttgtattatgt

SEQ ID NO: 364 - DNA, 24 bp, Artificial Sequence
  Description of Artificial SequenceArtificially Synthesized DNA Sequence
tgttaagaagtttagttaaagttg

SEQ ID NO: 365 - DNA, 24 bp, Artificial Sequence
  Description of Artificial SequenceArtificially Synthesized DNA Sequence
tttaagtataagattgtgtgagat

SEQ ID NO: 366 - DNA, 24 bp, Artificial Sequence
  Description of Artificial SequenceArtificially Synthesized DNA Sequence
agatatttgatagatagaagaaag

SEQ ID NO: 367 - DNA, 24 bp, Artificial Sequence
  Description of Artificial SequenceArtificially Synthesized DNA Sequence
atttagagttgtaagaagatattg

SEQ ID NO: 368 - DNA, 24 bp, Artificial Sequence
  Description of Artificial SequenceArtificially Synthesized DNA Sequence
gagaaattgtaattgttagagtat

SEQ ID NO: 369 - DNA, 24 bp, Artificial Sequence
  Description of Artificial SequenceArtificially Synthesized DNA Sequence
gaagtatatgttaagatgtaatag

SEQ ID NO: 370 - DNA, 24 bp, Artificial Sequence
  Description of Artificial SequenceArtificially Synthesized DNA Sequence
aatattgaagatgtagtgagttat

SEQ ID NO: 371 - DNA, 24 bp, Artificial Sequence
  Description of Artificial SequenceArtificially Synthesized DNA Sequence
gagtttagaaatgataaagaattg

SEQ ID NO: 372 - DNA, 24 bp, Artificial Sequence
  Description of Artificial SequenceArtificially Synthesized DNA Sequence
taagaaatgagttatatgttgaga

SEQ ID NO: 373 - DNA, 24 bp, Artificial Sequence
  Description of Artificial SequenceArtificially Synthesized DNA Sequence
ttgatataagaagttgtgataagt

SEQ ID NO: 374 - DNA, 24 bp, Artificial Sequence
  Description of Artificial SequenceArtificially Synthesized DNA Sequence
aagtgtttaatgtaagagaatgaa

SEQ ID NO: 375 - DNA, 24 bp, Artificial Sequence
  Description of Artificial SequenceArtificially Synthesized DNA Sequence
gttgtgagaattagaaatagtata

SEQ ID NO: 376 - DNA, 24 bp, Artificial Sequence
  Description of Artificial SequenceArtificially Synthesized DNA Sequence
tttagtttgatgtgtttatgagat

SEQ ID NO: 377 - DNA, 24 bp, Artificial Sequence
  Description of Artificial SequenceArtificially Synthesized DNA Sequence
gtaattgaaagtatgagtagtaat

SEQ ID NO: 378 - DNA, 24 bp, Artificial Sequence
  Description of Artificial SequenceArtificially Synthesized DNA Sequence
tagttgaataagattgagagaaat

SEQ ID NO: 379 - DNA, 24 bp, Artificial Sequence
  Description of Artificial SequenceArtificially Synthesized DNA Sequence
ttaagtgaagtgttgtttattgaa

SEQ ID NO: 380 - DNA, 24 bp, Artificial Sequence
  Description of Artificial SequenceArtificially Synthesized DNA Sequence
attgatttgttgaaataagtgttg

SEQ ID NO: 381 - DNA, 24 bp, Artificial Sequence
  Description of Artificial SequenceArtificially Synthesized DNA Sequence
tgaattgttgataagttatgaaga

SEQ ID NO: 382 - DNA, 24 bp, Artificial Sequence
  Description of Artificial SequenceArtificially Synthesized DNA Sequence
gtttgttattgagtaagttgaatt

SEQ ID NO: 383 - DNA, 24 bp, Artificial Sequence
  Description of Artificial SequenceArtificially Synthesized DNA Sequence
tgatttagtatgtattagagttga

SEQ ID NO: 384 - DNA, 24 bp, Artificial Sequence
  Description of Artificial SequenceArtificially Synthesized DNA Sequence
taaatagagatgagaataagaaag

SEQ ID NO: 385 - DNA, 24 bp, Artificial Sequence
  Description of Artificial SequenceArtificially Synthesized DNA Sequence
agaatgttatatgtagagaaattg

SEQ ID NO: 386 - DNA, 24 bp, Artificial Sequence
  Description of Artificial SequenceArtificially Synthesized DNA Sequence
atttatgtagttgagagtgataaa

SEQ ID NO: 387 - DNA, 24 bp, Artificial Sequence
  Description of Artificial SequenceArtificially Synthesized DNA Sequence
gtaaagatagtttgagtaatttga

SEQ ID NO: 388 - DNA, 24 bp, Artificial Sequence
  Description of Artificial SequenceArtificially Synthesized DNA Sequence
gaaatagtataatgttaagtgaga

SEQ ID NO: 389 - DNA, 24 bp, Artificial Sequence
  Description of Artificial SequenceArtificially Synthesized DNA Sequence
attgtatattgtgttgaagaaagt

SEQ ID NO: 390 - DNA, 24 bp, Artificial Sequence
  Description of Artificial SequenceArtificially Synthesized DNA Sequence
gagttaagtgtaaatgaaatgtaa

SEQ ID NO: 391 - DNA, 24 bp, Artificial Sequence
  Description of Artificial SequenceArtificially Synthesized DNA Sequence
atagattgtgtgaaagaaagaatt

SEQ ID NO: 392 - DNA, 24 bp, Artificial Sequence
  Description of Artificial SequenceArtificially Synthesized DNA Sequence
ttaatagaagtttgtagtatgatg

SEQ ID NO: 393 - DNA, 24 bp, Artificial Sequence
  Description of Artificial SequenceArtificially Synthesized DNA Sequence
ttgtatgtgagaataaagtttagt

SEQ ID NO: 394 - DNA, 24 bp, Artificial Sequence
  Description of Artificial SequenceArtificially Synthesized DNA Sequence
gtgattagatatgatgatatgaat

SEQ ID NO: 395 - DNA, 24 bp, Artificial Sequence
  Description of Artificial SequenceArtificially Synthesized DNA Sequence
tgaagaagaatttagatttgtaag

SEQ ID NO: 396 - DNA, 24 bp, Artificial Sequence
  Description of Artificial SequenceArtificially Synthesized DNA Sequence
tgtatgattattgattagtgtgtt

SEQ ID NO: 397 - DNA, 24 bp, Artificial Sequence
  Description of Artificial SequenceArtificially Synthesized DNA Sequence
tgtgaaagagaatgatagatattt

SEQ ID NO: 398 - DNA, 24 bp, Artificial Sequence
  Description of Artificial SequenceArtificially Synthesized DNA Sequence
aattgaaatgagtgtgtttaagaa

SEQ ID NO: 399 - DNA, 24 bp, Artificial Sequence
  Description of Artificial SequenceArtificially Synthesized DNA Sequence
attatagagttagtttagaatgag

SEQ ID NO: 400 - DNA, 24 bp, Artificial Sequence
  Description of Artificial SequenceArtificially Synthesized DNA Sequence
aaagatagaaattgagtgtatgat

SEQ ID NO: 401 - DNA, 24 bp, Artificial Sequence
  Description of Artificial SequenceArtificially Synthesized DNA Sequence
gtagtttgttaatgttgtataatg

SEQ ID NO: 402 - DNA, 24 bp, Artificial Sequence
  Description of Artificial SequenceArtificially Synthesized DNA Sequence
agagatattagaatgtaagaatag

SEQ ID NO: 403 - DNA, 24 bp, Artificial Sequence
  Description of Artificial SequenceArtificially Synthesized DNA Sequence
agaagtttgaaatatgatagaatg

SEQ ID NO: 404 - DNA, 24 bp, Artificial Sequence
  Description of Artificial SequenceArtificially Synthesized DNA Sequence
tagaatgtaaagtttagtatagag

SEQ ID NO: 405 - DNA, 24 bp, Artificial Sequence
  Description of Artificial SequenceArtificially Synthesized DNA Sequence
agtagatgtatgttaatgtgaata

SEQ ID NO: 406 - DNA, 24 bp, Artificial Sequence
  Description of Artificial SequenceArtificially Synthesized DNA Sequence
tgaaagtgaaatatgaaatgttgt

SEQ ID NO: 407 - DNA, 24 bp, Artificial Sequence
  Description of Artificial SequenceArtificially Synthesized DNA Sequence
atagtatattgagtttgtatgaag

SEQ ID NO: 408 - DNA, 24 bp, Artificial Sequence
  Description of Artificial SequenceArtificially Synthesized DNA Sequence
gaagaaatgtttgtagaataagta

SEQ ID NO: 409 - DNA, 24 bp, Artificial Sequence
  Description of Artificial SequenceArtificially Synthesized DNA Sequence
aatgagtattgaagaaatgtatag

SEQ ID NO: 410 - DNA, 24 bp, Artificial Sequence
  Description of Artificial SequenceArtificially Synthesized DNA Sequence
gtgatagaatttgtgtttaatgaa

SEQ ID NO: 411 - DNA, 24 bp, Artificial Sequence
  Description of Artificial SequenceArtificially Synthesized DNA Sequence
tgtagtatgaagaataatgaaatg

SEQ ID NO: 412 - DNA, 24 bp, Artificial Sequence
  Description of Artificial SequenceArtificially Synthesized DNA Sequence
atagaagttaatgataattgtgtg

SEQ ID NO: 413 - DNA, 24 bp, Artificial Sequence
  Description of Artificial SequenceArtificially Synthesized DNA Sequence
gtgattgtaagtaagtaaagataa

SEQ ID NO: 414 - DNA, 24 bp, Artificial Sequence
  Description of Artificial SequenceArtificially Synthesized DNA Sequence
tatgtagtttgtgttatttgaaga

SEQ ID NO: 415 - DNA, 24 bp, Artificial Sequence
  Description of Artificial SequenceArtificially Synthesized DNA Sequence
tgagtaagtttgtatgtttaagta

SEQ ID NO: 416 - DNA, 24 bp, Artificial Sequence
  Description of Artificial SequenceArtificially Synthesized DNA Sequence
taaatgtatgagtgtgtaaagaaa

SEQ ID NO: 417 - DNA, 24 bp, Artificial Sequence
  Description of Artificial SequenceArtificially Synthesized DNA Sequence
gtaagagtattgaaattagtaaga

SEQ ID NO: 418 - DNA, 24 bp, Artificial Sequence
  Description of Artificial SequenceArtificially Synthesized DNA Sequence
gttgagtgtaaagattattgataa

SEQ ID NO: 419 - DNA, 24 bp, Artificial Sequence
  Description of Artificial SequenceArtificially Synthesized DNA Sequence
agtatgagttattagataaagtga

SEQ ID NO: 420 - DNA, 24 bp, Artificial Sequence
  Description of Artificial SequenceArtificially Synthesized DNA Sequence
atttgttatagagttgtgttgtat

SEQ ID NO: 421 - DNA, 24 bp, Artificial Sequence
  Description of Artificial SequenceArtificially Synthesized DNA Sequence
taattagtagtgtgttgaaatttg

SEQ ID NO: 422 - DNA, 24 bp, Artificial Sequence
  Description of Artificial SequenceArtificially Synthesized DNA Sequence
tgtattgagattgttattgtattg

SEQ ID NO: 423 - DNA, 24 bp, Artificial Sequence
  Description of Artificial SequenceArtificially Synthesized DNA Sequence
gttattagaagagataattgagtt

SEQ ID NO: 424 - DNA, 24 bp, Artificial Sequence
  Description of Artificial SequenceArtificially Synthesized DNA Sequence
ttgagttgtgattaagtagtatat

SEQ ID NO: 425 - DNA, 24 bp, Artificial Sequence
  Description of Artificial SequenceArtificially Synthesized DNA Sequence
gatagtataatgattgaagtaatg

SEQ ID NO: 426 - DNA, 24 bp, Artificial Sequence
  Description of Artificial SequenceArtificially Synthesized DNA Sequence
gtgaaagatatttgagagataaat

SEQ ID NO: 427 - DNA, 24 bp, Artificial Sequence
  Description of Artificial SequenceArtificially Synthesized DNA Sequence
agttatgatttgaagaaattgttg

SEQ ID NO: 428 - DNA, 24 bp, Artificial Sequence
  Description of Artificial SequenceArtificially Synthesized DNA Sequence
gtaagtatttgaatttgatgagtt

SEQ ID NO: 429 - DNA, 24 bp, Artificial Sequence
  Description of Artificial SequenceArtificially Synthesized DNA Sequence
taatagtgttataagtgaaagagt

SEQ ID NO: 430 - DNA, 24 bp, Artificial Sequence
  Description of Artificial SequenceArtificially Synthesized DNA Sequence
aaatgaattgatgtgtatatgaag

SEQ ID NO: 431 - DNA, 24 bp, Artificial Sequence
  Description of Artificial SequenceArtificially Synthesized DNA Sequence
agaaagtgagttgttaagtattta

SEQ ID NO: 432 - DNA, 24 bp, Artificial Sequence
  Description of Artificial SequenceArtificially Synthesized DNA Sequence
tttatgtgtgaattgtgtatatag

SEQ ID NO: 433 - DNA, 24 bp, Artificial Sequence
  Description of Artificial SequenceArtificially Synthesized DNA Sequence
gtaatatgatagaaatgtaaagag

SEQ ID NO: 434 - DNA, 24 bp, Artificial Sequence
  Description of Artificial SequenceArtificially Synthesized DNA Sequence
gagaattgtttaaagatagttgta

SEQ ID NO: 435 - DNA, 24 bp, Artificial Sequence
  Description of Artificial SequenceArtificially Synthesized DNA Sequence
gaatttgttaagaatgagtttgat

SEQ ID NO: 436 - DNA, 24 bp, Artificial Sequence
  Description of Artificial SequenceArtificially Synthesized DNA Sequence
atagtgatgattaaagagaatttg

SEQ ID NO: 437 - DNA, 24 bp, Artificial Sequence
  Description of Artificial SequenceArtificially Synthesized DNA Sequence
atagatgtttagttgagattattg

SEQ ID NO: 438 - DNA, 24 bp, Artificial Sequence
  Description of Artificial SequenceArtificially Synthesized DNA Sequence
aagagtgtaaatagaaagtgatat

SEQ ID NO: 439 - DNA, 24 bp, Artificial Sequence
  Description of Artificial SequenceArtificially Synthesized DNA Sequence
tgtgtattgattgttgagataaat

SEQ ID NO: 440 - DNA, 24 bp, Artificial Sequence
  Description of Artificial SequenceArtificially Synthesized DNA Sequence
tagtatagtgagaaagagttaaat

SEQ ID NO: 441 - DNA, 24 bp, Artificial Sequence
  Description of Artificial SequenceArtificially Synthesized DNA Sequence
aaagataagaaagagatgatgttt

SEQ ID NO: 442 - DNA, 24 bp, Artificial Sequence
  Description of Artificial SequenceArtificially Synthesized DNA Sequence
gaagttattgaaatagagaagtat

SEQ ID NO: 443 - DNA, 24 bp, Artificial Sequence
  Description of Artificial SequenceArtificially Synthesized DNA Sequence
atgtatgtatagaaagagtaaatg

SEQ ID NO: 444 - DNA, 24 bp, Artificial Sequence
  Description of Artificial SequenceArtificially Synthesized DNA Sequence
gatgtttgtaaagattgaaattga

SEQ ID NO: 445 - DNA, 24 bp, Artificial Sequence
  Description of Artificial SequenceArtificially Synthesized DNA Sequence
aatttagagagtatttgtgttgta

SEQ ID NO: 446 - DNA, 24 bp, Artificial Sequence
  Description of Artificial SequenceArtificially Synthesized DNA Sequence
aatttgtttgaaagaaagtaagtg

SEQ ID NO: 447 - DNA, 24 bp, Artificial Sequence
  Description of Artificial SequenceArtificially Synthesized DNA Sequence
aaagagtagtgttattgttagata

SEQ ID NO: 448 - DNA, 24 bp, Artificial Sequence
  Description of Artificial SequenceArtificially Synthesized DNA Sequence
gtatgttgtatatgttgttgatat

SEQ ID NO: 449 - DNA, 24 bp, Artificial Sequence
  Description of Artificial SequenceArtificially Synthesized DNA Sequence
gtagaatttgttgagtatttgtaa

SEQ ID NO: 450 - DNA, 24 bp, Artificial Sequence
  Description of Artificial SequenceArtificially Synthesized DNA Sequence
atgaatttagttagtgtaagaaag

SEQ ID NO: 451 - DNA, 24 bp, Artificial Sequence
  Description of Artificial SequenceArtificially Synthesized DNA Sequence
atgataagaaatgttgatgaagta

SEQ ID NO: 452 - DNA, 24 bp, Artificial Sequence
  Description of Artificial SequenceArtificially Synthesized DNA Sequence
ttgatgatgaagataatgtagata

SEQ ID NO: 453 - DNA, 24 bp, Artificial Sequence
  Description of Artificial SequenceArtificially Synthesized DNA Sequence
agatgatatgatatagattagatg

SEQ ID NO: 454 - DNA, 24 bp, Artificial Sequence
  Description of Artificial SequenceArtificially Synthesized DNA Sequence
ttgaaagttagaaagatagatgtt

SEQ ID NO: 455 - DNA, 24 bp, Artificial Sequence
  Description of Artificial SequenceArtificially Synthesized DNA Sequence
gtttaatgttagttagaaagtaag

SEQ ID NO: 456 - DNA, 24 bp, Artificial Sequence
  Description of Artificial SequenceArtificially Synthesized DNA Sequence
gagatttaagtttgaagtgaaata

SEQ ID NO: 457 - DNA, 24 bp, Artificial Sequence
  Description of Artificial SequenceArtificially Synthesized DNA Sequence
tttgttagtagttgttataagaga

SEQ ID NO: 458 - DNA, 24 bp, Artificial Sequence
  Description of Artificial SequenceArtificially Synthesized DNA Sequence
tatgagaatagtttgttagtgaat

SEQ ID NO: 459 - DNA, 24 bp, Artificial Sequence
  Description of Artificial SequenceArtificially Synthesized DNA Sequence
ttgaaagtttaaagaagagataag

SEQ ID NO: 460 - DNA, 24 bp, Artificial Sequence
  Description of Artificial SequenceArtificially Synthesized DNA Sequence
aagtgagttgaaatgaaatatgtt

SEQ ID NO: 461 - DNA, 24 bp, Artificial Sequence
  Description of Artificial SequenceArtificially Synthesized DNA Sequence
gttagaaatgaaatgagtagttat

SEQ ID NO: 462 - DNA, 24 bp, Artificial Sequence
  Description of Artificial SequenceArtificially Synthesized DNA Sequence
taagtattgtatttgtgtgtgtat

SEQ ID NO: 463 - DNA, 24 bp, Artificial Sequence
  Description of Artificial SequenceArtificially Synthesized DNA Sequence
tgtattagtaaagaagagagaata

SEQ ID NO: 464 - DNA, 24 bp, Artificial Sequence
  Description of Artificial SequenceArtificially Synthesized DNA Sequence
gagaagagaaataagttgaaataa

SEQ ID NO: 465 - DNA, 24 bp, Artificial Sequence
  Description of Artificial SequenceArtificially Synthesized DNA Sequence
gtaaagtagaaatagaattgagtt

SEQ ID NO: 466 - DNA, 24 bp, Artificial Sequence
  Description of Artificial SequenceArtificially Synthesized DNA Sequence
gtgtgttatttgtttgtaaagtat

SEQ ID NO: 467 - DNA, 24 bp, Artificial Sequence
  Description of Artificial SequenceArtificially Synthesized DNA Sequence
tttgatgtatgaatatagtatgag

SEQ ID NO: 468 - DNA, 24 bp, Artificial Sequence
  Description of Artificial SequenceArtificially Synthesized DNA Sequence
aagattgtgtgaatagttgaaatt

SEQ ID NO: 469 - DNA, 24 bp, Artificial Sequence
  Description of Artificial SequenceArtificially Synthesized DNA Sequence
tataaagtttgaagatgagtgata

SEQ ID NO: 470 - DNA, 24 bp, Artificial Sequence
  Description of Artificial SequenceArtificially Synthesized DNA Sequence
agataaagagatttaagatgtatg

SEQ ID NO: 471 - DNA, 24 bp, Artificial Sequence
  Description of Artificial SequenceArtificially Synthesized DNA Sequence
gaagaattaagttgagaattaaga

SEQ ID NO: 472 - DNA, 24 bp, Artificial Sequence
  Description of Artificial SequenceArtificially Synthesized DNA Sequence
tagagaaatttgataaagaaagag

SEQ ID NO: 473 - DNA, 24 bp, Artificial Sequence
  Description of Artificial SequenceArtificially Synthesized DNA Sequence
aaagtttatgaagttattgagtag

SEQ ID NO: 474 - DNA, 24 bp, Artificial Sequence
  Description of Artificial SequenceArtificially Synthesized DNA Sequence
aaatagtgtaagtaaagagatgat

SEQ ID NO: 475 - DNA, 24 bp, Artificial Sequence
  Description of Artificial SequenceArtificially Synthesized DNA Sequence
tatgatgatttagttataagagtg

SEQ ID NO: 476 - DNA, 24 bp, Artificial Sequence
  Description of Artificial SequenceArtificially Synthesized DNA Sequence
tagataaatgttatgatgagtaag

SEQ ID NO: 477 - DNA, 24 bp, Artificial Sequence
  Description of Artificial SequenceArtificially Synthesized DNA Sequence
agattgattgtgatgatttgtata

SEQ ID NO: 478 - DNA, 24 bp, Artificial Sequence
  Description of Artificial SequenceArtificially Synthesized DNA Sequence
ttaagaagaattgtatatgagagt

SEQ ID NO: 479 - DNA, 24 bp, Artificial Sequence
  Description of Artificial SequenceArtificially Synthesized DNA Sequence
gtagaatgtttagagttgaatata

SEQ ID NO: 480 - DNA, 24 bp, Artificial Sequence
  Description of Artificial SequenceArtificially Synthesized DNA Sequence
gagaaatagtaagaagtaaataga

SEQ ID NO: 481 - DNA, 24 bp, Artificial Sequence
  Description of Artificial SequenceArtificially Synthesized DNA Sequence
attgaagttgttatgtgaagattt

SEQ ID NO: 482 - DNA, 24 bp, Artificial Sequence
  Description of Artificial SequenceArtificially Synthesized DNA Sequence
taaatgttgtgtagagtaattaga

SEQ ID NO: 483 - DNA, 24 bp, Artificial Sequence
  Description of Artificial SequenceArtificially Synthesized DNA Sequence
aaataagagtttgagaagttgttt

SEQ ID NO: 484 - DNA, 24 bp, Artificial Sequence
  Description of Artificial SequenceArtificially Synthesized DNA Sequence
agttgtaataagaagtgatttaag

SEQ ID NO: 485 - DNA, 24 bp, Artificial Sequence
  Description of Artificial SequenceArtificially Synthesized DNA Sequence
gttagaatgtatatagagttagat

SEQ ID NO: 486 - DNA, 24 bp, Artificial Sequence
  Description of Artificial SequenceArtificially Synthesized DNA Sequence
ttgatattgaaagagaaagttatg

SEQ ID NO: 487 - DNA, 24 bp, Artificial Sequence
  Description of Artificial SequenceArtificially Synthesized DNA Sequence
ttaaagagagaaatgtttgattag

SEQ ID NO: 488 - DNA, 24 bp, Artificial Sequence
  Description of Artificial SequenceArtificially Synthesized DNA Sequence
tgtgaatttgagtattagtaagaa

SEQ ID NO: 489 - DNA, 24 bp, Artificial Sequence
  Description of Artificial SequenceArtificially Synthesized DNA Sequence
taatttgaatgtgaaagttgttag

SEQ ID NO: 490 - DNA, 24 bp, Artificial Sequence
  Description of Artificial SequenceArtificially Synthesized DNA Sequence
atgtgtttgaaagatgatgattta

SEQ ID NO: 491 - DNA, 24 bp, Artificial Sequence
  Description of Artificial SequenceArtificially Synthesized DNA Sequence
aagttatgttgatattgagtgaaa

SEQ ID NO: 492 - DNA, 24 bp, Artificial Sequence
  Description of Artificial SequenceArtificially Synthesized DNA Sequence
tagataaagaagatagagatttag

SEQ ID NO: 493 - DNA, 24 bp, Artificial Sequence
  Description of Artificial SequenceArtificially Synthesized DNA Sequence
gatgaatgtagatatatgtaatga

SEQ ID NO: 494 - DNA, 24 bp, Artificial Sequence
  Description of Artificial SequenceArtificially Synthesized DNA Sequence
gaagaatagtttatgtaaatgatg

SEQ ID NO: 495 - DNA, 24 bp, Artificial Sequence
  Description of Artificial SequenceArtificially Synthesized DNA Sequence
gtagtatatagttaaagatgagtt

SEQ ID NO: 496 - DNA, 24 bp, Artificial Sequence
  Description of Artificial SequenceArtificially Synthesized DNA Sequence
gttatttgtgtatgattatgattg

SEQ ID NO: 497 - DNA, 24 bp, Artificial Sequence
  Description of Artificial SequenceArtificially Synthesized DNA Sequence
agagattagaaattgagagaatta

SEQ ID NO: 498 - DNA, 24 bp, Artificial Sequence
  Description of Artificial SequenceArtificially Synthesized DNA Sequence
gtatgatagagtttatagtgataa

SEQ ID NO: 499 - DNA, 24 bp, Artificial Sequence
  Description of Artificial SequenceArtificially Synthesized DNA Sequence
gttagaaagaatgaaattgaagta

SEQ ID NO: 500 - DNA, 24 bp, Artificial Sequence
  Description of Artificial SequenceArtificially Synthesized DNA Sequence
aagaatgagaatatagagatgaat

SEQ ID NO: 501 - DNA, 24 bp, Artificial Sequence
  Description of Artificial SequenceArtificially Synthesized DNA Sequence
aaagagaatagtgtttaagaagat

SEQ ID NO: 502 - DNA, 24 bp, Artificial Sequence
  Description of Artificial SequenceArtificially Synthesized DNA Sequence
gatgtgttattgatagaaattaga

SEQ ID NO: 503 - DNA, 24 bp, Artificial Sequence
  Description of Artificial SequenceArtificially Synthesized DNA Sequence
tagagttatagagatattgtatga

SEQ ID NO: 504 - DNA, 24 bp, Artificial Sequence
  Description of Artificial SequenceArtificially Synthesized DNA Sequence
gagagttgaataagttaaagatat

SEQ ID NO: 505 - DNA, 24 bp, Artificial Sequence
  Description of Artificial SequenceArtificially Synthesized DNA Sequence
agatatgaaatagattgttagaga

SEQ ID NO: 506 - DNA, 24 bp, Artificial Sequence
  Description of Artificial SequenceArtificially Synthesized DNA Sequence
gagtgaatagaaagatatgttaat

SEQ ID NO: 507 - DNA, 24 bp, Artificial Sequence
  Description of Artificial SequenceArtificially Synthesized DNA Sequence
aaagagatattgaagagaataaag

SEQ ID NO: 508 - DNA, 24 bp, Artificial Sequence
  Description of Artificial SequenceArtificially Synthesized DNA Sequence
gttatagaataagttgtaaagtgt

SEQ ID NO: 509 - DNA, 24 bp, Artificial Sequence
  Description of Artificial SequenceArtificially Synthesized DNA Sequence
tgatagtatgataatgtgtttatg

SEQ ID NO: 510 - DNA, 24 bp, Artificial Sequence
  Description of Artificial SequenceArtificially Synthesized DNA Sequence
tttgttgttaagtatgtgatttag

SEQ ID NO: 511 - DNA, 24 bp, Artificial Sequence
  Description of Artificial SequenceArtificially Synthesized DNA Sequence
taaagtgttgtgttaaagattaag

SEQ ID NO: 512 - DNA, 24 bp, Artificial Sequence
  Description of Artificial SequenceArtificially Synthesized DNA Sequence
tgtgtttgattgattaatgttatg

SEQ ID NO: 513 - DNA, 24 bp, Artificial Sequence
  Description of Artificial SequenceArtificially Synthesized DNA Sequence
attaatgaatgagtgttgtaatgt

SEQ ID NO: 514 - DNA, 24 bp, Artificial Sequence
  Description of Artificial SequenceArtificially Synthesized DNA Sequence
tagatgtttgtgagtttgatatta

SEQ ID NO: 515 - DNA, 24 bp, Artificial Sequence
  Description of Artificial SequenceArtificially Synthesized DNA Sequence
gaatgaatagtaatagatgatttg

SEQ ID NO: 516 - DNA, 24 bp, Artificial Sequence
  Description of Artificial SequenceArtificially Synthesized DNA Sequence
aatagtgtgttgttatatgattag

SEQ ID NO: 517 - DNA, 24 bp, Artificial Sequence
  Description of Artificial SequenceArtificially Synthesized DNA Sequence
tagattagaagatgttgtgtatta

SEQ ID NO: 518 - DNA, 24 bp, Artificial Sequence
  Description of Artificial SequenceArtificially Synthesized DNA Sequence
aatgtgtgtgttaaatgaatttgt

SEQ ID NO: 519 - DNA, 24 bp, Artificial Sequence
  Description of Artificial SequenceArtificially Synthesized DNA Sequence
gaattaagtatatgagtgtagaaa

SEQ ID NO: 520 - DNA, 24 bp, Artificial Sequence
  Description of Artificial SequenceArtificially Synthesized DNA Sequence
ttattgtgtgtaagtagtgtaaat

SEQ ID NO: 521 - DNA, 24 bp, Artificial Sequence
  Description of Artificial SequenceArtificially Synthesized DNA Sequence
gtagtaaagagaattgtttagtat

SEQ ID NO: 522 - DNA, 24 bp, Artificial Sequence
  Description of Artificial SequenceArtificially Synthesized DNA Sequence
aagtttgtaagaagtagttgaata

SEQ ID NO: 523 - DNA, 24 bp, Artificial Sequence
  Description of Artificial SequenceArtificially Synthesized DNA Sequence
agttatagtatagtagtatagaga

SEQ ID NO: 524 - DNA, 24 bp, Artificial Sequence
  Description of Artificial SequenceArtificially Synthesized DNA Sequence
gaaagaaatgtgtatagtttaatg

SEQ ID NO: 525 - DNA, 24 bp, Artificial Sequence
  Description of Artificial SequenceArtificially Synthesized DNA Sequence
ttgtgagtaatgaatgatgtatta

SEQ ID NO: 526 - DNA, 24 bp, Artificial Sequence
  Description of Artificial SequenceArtificially Synthesized DNA Sequence
gtagagttgtaaatagagaataaa

SEQ ID NO: 527 - DNA, 24 bp, Artificial Sequence
  Description of Artificial SequenceArtificially Synthesized DNA Sequence
attaatgtagattgtaagagatag

SEQ ID NO: 528 - DNA, 24 bp, Artificial Sequence
  Description of Artificial SequenceArtificially Synthesized DNA Sequence
ttagtgtgtttgtagatagaatta

SEQ ID NO: 529 - DNA, 24 bp, Artificial Sequence
  Description of Artificial SequenceArtificially Synthesized DNA Sequence
agagagtttgtgtatatgtataaa

SEQ ID NO: 530 - DNA, 24 bp, Artificial Sequence
  Description of Artificial SequenceArtificially Synthesized DNA Sequence
ttaagtttagtgagatttgttaag

SEQ ID NO: 531 - DNA, 24 bp, Artificial Sequence
  Description of Artificial SequenceArtificially Synthesized DNA Sequence
atgaagtttattgaatagtagtga

SEQ ID NO: 532 - DNA, 24 bp, Artificial Sequence
  Description of Artificial SequenceArtificially Synthesized DNA Sequence
atatttgtgttgtatgtttgtgaa

SEQ ID NO: 533 - DNA, 24 bp, Artificial Sequence
  Description of Artificial SequenceArtificially Synthesized DNA Sequence
aaagtgtttatagaagatttgatg

SEQ ID NO: 534 - DNA, 24 bp, Artificial Sequence
  Description of Artificial SequenceArtificially Synthesized DNA Sequence
aagagatatgatttgttagttgta

SEQ ID NO: 535 - DNA, 24 bp, Artificial Sequence
  Description of Artificial SequenceArtificially Synthesized DNA Sequence
aagaagaaatgagtgataatgtaa

SEQ ID NO: 536 - DNA, 24 bp, Artificial Sequence
  Description of Artificial SequenceArtificially Synthesized DNA Sequence
tagtgtttgatatgttaagaagtt

SEQ ID NO: 537 - DNA, 24 bp, Artificial Sequence
  Description of Artificial SequenceArtificially Synthesized DNA Sequence
gtagaaagtgatagattagtaata

SEQ ID NO: 538 - DNA, 24 bp, Artificial Sequence
  Description of Artificial SequenceArtificially Synthesized DNA Sequence
gataaatgttaagttagtatgatg

SEQ ID NO: 539 - DNA, 24 bp, Artificial Sequence
  Description of Artificial SequenceArtificially Synthesized DNA Sequence
agattagaagaattgtttagaatg

SEQ ID NO: 540 - DNA, 24 bp, Artificial Sequence
  Description of Artificial SequenceArtificially Synthesized DNA Sequence
atatttgagaagtgtgaaatgaat

SEQ ID NO: 541 - DNA, 24 bp, Artificial Sequence
  Description of Artificial SequenceArtificially Synthesized DNA Sequence
tgagtaaatagtttatgagtagta

SEQ ID NO: 542 - DNA, 24 bp, Artificial Sequence
  Description of Artificial SequenceArtificially Synthesized DNA Sequence
ttagagagtagataaagatttgat

SEQ ID NO: 543 - DNA, 24 bp, Artificial Sequence
  Description of Artificial SequenceArtificially Synthesized DNA Sequence
attgtttaagttgttgataagatg

SEQ ID NO: 544 - DNA, 24 bp, Artificial Sequence
  Description of Artificial SequenceArtificially Synthesized DNA Sequence
gttgtaaagttaaagtgtgaattt

SEQ ID NO: 545 - DNA, 24 bp, Artificial Sequence
  Description of Artificial SequenceArtificially Synthesized DNA Sequence
atagattgtgtgtttgttatagta

SEQ ID NO: 546 - DNA, 24 bp, Artificial Sequence
  Description of Artificial SequenceArtificially Synthesized DNA Sequence
gtaagttattgagaatgataatag

SEQ ID NO: 547 - DNA, 24 bp, Artificial Sequence
  Description of Artificial SequenceArtificially Synthesized DNA Sequence
tagattagttgataagtgtgtaat

SEQ ID NO: 548 - DNA, 24 bp, Artificial Sequence
  Description of Artificial SequenceArtificially Synthesized DNA Sequence
aaatgtaaatgaagagtgtttgtt

SEQ ID NO: 549 - DNA, 24 bp, Artificial Sequence
  Description of Artificial SequenceArtificially Synthesized DNA Sequence
gatagaagaaatgtatatagtgat

SEQ ID NO: 550 - DNA, 24 bp, Artificial Sequence
  Description of Artificial SequenceArtificially Synthesized DNA Sequence
tatagagtgtatgttatgataaag

SEQ ID NO: 551 - DNA, 24 bp, Artificial Sequence
  Description of Artificial SequenceArtificially Synthesized DNA Sequence
tatgaagtgataagatgaagaatt

SEQ ID NO: 552 - DNA, 24 bp, Artificial Sequence
  Description of Artificial SequenceArtificially Synthesized DNA Sequence
tgttgagaatagtaagagaattta

SEQ ID NO: 553 - DNA, 24 bp, Artificial Sequence
  Description of Artificial SequenceArtificially Synthesized DNA Sequence
tagataatgtgaagtaataagtga

SEQ ID NO: 554 - DNA, 24 bp, Artificial Sequence
  Description of Artificial SequenceArtificially Synthesized DNA Sequence
gtattatgatgatagtagtaagta

SEQ ID NO: 555 - DNA, 24 bp, Artificial Sequence
  Description of Artificial SequenceArtificially Synthesized DNA Sequence
agatatgatttagtattgaatgtg

SEQ ID NO: 556 - DNA, 24 bp, Artificial Sequence
  Description of Artificial SequenceArtificially Synthesized DNA Sequence
aattaagtttgtagagtgatttga

SEQ ID NO: 557 - DNA, 24 bp, Artificial Sequence
  Description of Artificial SequenceArtificially Synthesized DNA Sequence
aagaaatagatgtagtaagatgtt

SEQ ID NO: 558 - DNA, 24 bp, Artificial Sequence
  Description of Artificial SequenceArtificially Synthesized DNA Sequence
ttgagaagttgttgtaataagaat

SEQ ID NO: 559 - DNA, 24 bp, Artificial Sequence
  Description of Artificial SequenceArtificially Synthesized DNA Sequence
agtgtgaaatagtgaaagtttaaa

SEQ ID NO: 560 - DNA, 24 bp, Artificial Sequence
  Description of Artificial SequenceArtificially Synthesized DNA Sequence
tttatgtagtagatttatgtgaag

SEQ ID NO: 561 - DNA, 24 bp, Artificial Sequence
  Description of Artificial SequenceArtificially Synthesized DNA Sequence
attaatgagaaattagtgtgttag

SEQ ID NO: 562 - DNA, 24 bp, Artificial Sequence
  Description of Artificial SequenceArtificially Synthesized DNA Sequence
atgttaatagtgatagtaaagtga

SEQ ID NO: 563 - DNA, 24 bp, Artificial Sequence
  Description of Artificial SequenceArtificially Synthesized DNA Sequence
tatgttgataaatgattatgagtg

SEQ ID NO: 564 - DNA, 24 bp, Artificial Sequence
  Description of Artificial SequenceArtificially Synthesized DNA Sequence
ttattagagttgtgtgtgatatat

SEQ ID NO: 565 - DNA, 24 bp, Artificial Sequence
  Description of Artificial SequenceArtificially Synthesized DNA Sequence
tgttgttatgattgagttagaata

SEQ ID NO: 566 - DNA, 24 bp, Artificial Sequence
  Description of Artificial SequenceArtificially Synthesized DNA Sequence
aatttgagttaagaagaagtgtaa

SEQ ID NO: 567 - DNA, 24 bp, Artificial Sequence
  Description of Artificial SequenceArtificially Synthesized DNA Sequence
aaagataaagttaagtgtttgtag

SEQ ID NO: 568 - DNA, 24 bp, Artificial Sequence
  Description of Artificial SequenceArtificially Synthesized DNA Sequence
tgttgagatgatattgtataagtt

SEQ ID NO: 569 - DNA, 24 bp, Artificial Sequence
  Description of Artificial SequenceArtificially Synthesized DNA Sequence
taaatagtgaatgagttatagagt

SEQ ID NO: 570 - DNA, 24 bp, Artificial Sequence
  Description of Artificial SequenceArtificially Synthesized DNA Sequence
atagatgttatgatagttagttag

SEQ ID NO: 571 - DNA, 24 bp, Artificial Sequence
  Description of Artificial SequenceArtificially Synthesized DNA Sequence
gttaagtgaagatatgtattgtta

SEQ ID NO: 572 - DNA, 24 bp, Artificial Sequence
  Description of Artificial SequenceArtificially Synthesized DNA Sequence
taagaaagtaaagtttgtagatgt

SEQ ID NO: 573 - DNA, 24 bp, Artificial Sequence
  Description of Artificial SequenceArtificially Synthesized DNA Sequence
aagagaaagtttgattgaataaag

SEQ ID NO: 574 - DNA, 24 bp, Artificial Sequence
  Description of Artificial SequenceArtificially Synthesized DNA Sequence
atattagatgtgagttatatgtgt

SEQ ID NO: 575 - DNA, 24 bp, Artificial Sequence
  Description of Artificial SequenceArtificially Synthesized DNA Sequence
agtttgagtttagtattgtgaata

SEQ ID NO: 576 - DNA, 24 bp, Artificial Sequence
  Description of Artificial SequenceArtificially Synthesized DNA Sequence
atgttaaatgagagattgtgtata

SEQ ID NO: 577 - DNA, 24 bp, Artificial Sequence
  Description of Artificial SequenceArtificially Synthesized DNA Sequence
taaatgttgtgattattgtgagat

SEQ ID NO: 578 - DNA, 24 bp, Artificial Sequence
  Description of Artificial SequenceArtificially Synthesized DNA Sequence
taagaattgaagtaagagttattg

SEQ ID NO: 579 - DNA, 24 bp, Artificial Sequence
  Description of Artificial SequenceArtificially Synthesized DNA Sequence
agagatagaattaagtttgttgat

SEQ ID NO: 580 - DNA, 24 bp, Artificial Sequence
  Description of Artificial SequenceArtificially Synthesized DNA Sequence
gaagaatgttaagaaatatgtaag

SEQ ID NO: 581 - DNA, 24 bp, Artificial Sequence
  Description of Artificial SequenceArtificially Synthesized DNA Sequence
tatttgtgattaagaagttgagaa

SEQ ID NO: 582 - DNA, 24 bp, Artificial Sequence
  Description of Artificial SequenceArtificially Synthesized DNA Sequence
agttagaatttgtgtagtagaatt

SEQ ID NO: 583 - DNA, 24 bp, Artificial Sequence
  Description of Artificial SequenceArtificially Synthesized DNA Sequence
aagtttattgttgatgttgtattg

SEQ ID NO: 584 - DNA, 24 bp, Artificial Sequence
  Description of Artificial SequenceArtificially Synthesized DNA Sequence
gaatgagtttaagagtttatagta

SEQ ID NO: 585 - DNA, 24 bp, Artificial Sequence
  Description of Artificial SequenceArtificially Synthesized DNA Sequence
agtgaagattgtatgtagtataaa

SEQ ID NO: 586 - DNA, 24 bp, Artificial Sequence
  Description of Artificial SequenceArtificially Synthesized DNA Sequence
agttgaaatgagtattaagtaatg

SEQ ID NO: 587 - DNA, 24 bp, Artificial Sequence
  Description of Artificial SequenceArtificially Synthesized DNA Sequence
atgtgttatttgagatgagtaatt

SEQ ID NO: 588 - DNA, 24 bp, Artificial Sequence
  Description of Artificial SequenceArtificially Synthesized DNA Sequence
aaatagtgttgttgaagttgttat

SEQ ID NO: 589 - DNA, 24 bp, Artificial Sequence
  Description of Artificial SequenceArtificially Synthesized DNA Sequence
gtagagaaagatatatgtagttta

SEQ ID NO: 590 - DNA, 24 bp, Artificial Sequence
  Description of Artificial SequenceArtificially Synthesized DNA Sequence
gagagtatttgatgaatgattata

SEQ ID NO: 591 - DNA, 24 bp, Artificial Sequence
  Description of Artificial SequenceArtificially Synthesized DNA Sequence
gagtataagtttagtgtatattga

SEQ ID NO: 592 - DNA, 24 bp, Artificial Sequence
  Description of Artificial SequenceArtificially Synthesized DNA Sequence
ataatgtgattattgattgagaga

SEQ ID NO: 593 - DNA, 24 bp, Artificial Sequence
  Description of Artificial SequenceArtificially Synthesized DNA Sequence
ttagttgttatgtgagagtaataa

SEQ ID NO: 594 - DNA, 24 bp, Artificial Sequence
  Description of Artificial SequenceArtificially Synthesized DNA Sequence
aaatgagtatattgaattgtgatg

SEQ ID NO: 595 - DNA, 24 bp, Artificial Sequence
  Description of Artificial SequenceArtificially Synthesized DNA Sequence
aattagaagtaagtagagtttaag

SEQ ID NO: 596 - DNA, 24 bp, Artificial Sequence
  Description of Artificial SequenceArtificially Synthesized DNA Sequence
tgtaagtttaaagtaagaaatgtg

SEQ ID NO: 597 - DNA, 24 bp, Artificial Sequence
  Description of Artificial SequenceArtificially Synthesized DNA Sequence
gaaatgataagttgatataagaag

SEQ ID NO: 598 - DNA, 24 bp, Artificial Sequence
  Description of Artificial SequenceArtificially Synthesized DNA Sequence
aatgagtagtttgtatttgagttt

SEQ ID NO: 599 - DNA, 24 bp, Artificial Sequence
  Description of Artificial SequenceArtificially Synthesized DNA Sequence
agtgaatgtaagattatgtatttg

SEQ ID NO: 600 - DNA, 24 bp, Artificial Sequence
  Description of Artificial SequenceArtificially Synthesized DNA Sequence
gtaattgaattgaaagataagtgt

SEQ ID NO: 601 - DNA, 24 bp, Artificial Sequence
  Description of Artificial SequenceArtificially Synthesized DNA Sequence
tatgtttaagtagtgaaatagagt

SEQ ID NO: 602 - DNA, 24 bp, Artificial Sequence
  Description of Artificial SequenceArtificially Synthesized DNA Sequence
gtattgaaattgaattagaagtag

SEQ ID NO: 603 - DNA, 24 bp, Artificial Sequence
  Description of Artificial SequenceArtificially Synthesized DNA Sequence
aatatgtaatgtagttgaaagtga

SEQ ID NO: 604 - DNA, 24 bp, Artificial Sequence
  Description of Artificial SequenceArtificially Synthesized DNA Sequence
tgaatattgagaattatgagagtt

SEQ ID NO: 605 - DNA, 24 bp, Artificial Sequence
  Description of Artificial SequenceArtificially Synthesized DNA Sequence
tagtgtaaatgatgaagaaagtat

SEQ ID NO: 606 - DNA, 24 bp, Artificial Sequence
  Description of Artificial SequenceArtificially Synthesized DNA Sequence
gtatgtgtaaagaaatttgatgta

SEQ ID NO: 607 - DNA, 24 bp, Artificial Sequence
  Description of Artificial SequenceArtificially Synthesized DNA Sequence
aattgtttgaaagtttgttgagaa

SEQ ID NO: 608 - DNA, 24 bp, Artificial Sequence
  Description of Artificial SequenceArtificially Synthesized DNA Sequence
aattgtttgagtagtattagtagt

SEQ ID NO: 609 - DNA, 24 bp, Artificial Sequence
  Description of Artificial SequenceArtificially Synthesized DNA Sequence
taattgagtttgaataagagagtt

SEQ ID NO: 610 - DNA, 24 bp, Artificial Sequence
  Description of Artificial SequenceArtificially Synthesized DNA Sequence
tgttgattgtaagtgtttattgtt

SEQ ID NO: 611 - DNA, 24 bp, Artificial Sequence
  Description of Artificial SequenceArtificially Synthesized DNA Sequence
gaaatttgtgagtatgtatttgaa

SEQ ID NO: 612 - DNA, 24 bp, Artificial Sequence
  Description of Artificial SequenceArtificially Synthesized DNA Sequence
taagaatgaatgtgaagtgaatat

SEQ ID NO: 613 - DNA, 24 bp, Artificial Sequence
  Description of Artificial SequenceArtificially Synthesized DNA Sequence
taatgtgaagtttgtgaaagatat

SEQ ID NO: 614 - DNA, 24 bp, Artificial Sequence
  Description of Artificial SequenceArtificially Synthesized DNA Sequence
ttgtatatgaaagtaagaagaagt

SEQ ID NO: 615 - DNA, 24 bp, Artificial Sequence
  Description of Artificial SequenceArtificially Synthesized DNA Sequence
tagagagaagaagaaataagaata

SEQ ID NO: 616 - DNA, 24 bp, Artificial Sequence
  Description of Artificial SequenceArtificially Synthesized DNA Sequence
atttgaaatgttaatgagagagat

SEQ ID NO: 617 - DNA, 24 bp, Artificial Sequence
  Description of Artificial SequenceArtificially Synthesized DNA Sequence
ttgtgtgtatatagtattagaatg

SEQ ID NO: 618 - DNA, 24 bp, Artificial Sequence
  Description of Artificial SequenceArtificially Synthesized DNA Sequence
attgttagtattgatgtgaagtta

SEQ ID NO: 619 - DNA, 24 bp, Artificial Sequence
  Description of Artificial SequenceArtificially Synthesized DNA Sequence
tgtttgtatttgaatgaaatgaag

SEQ ID NO: 620 - DNA, 24 bp, Artificial Sequence
  Description of Artificial SequenceArtificially Synthesized DNA Sequence
tgttagattgtgttaaatgtagtt

SEQ ID NO: 621 - DNA, 24 bp, Artificial Sequence
  Description of Artificial SequenceArtificially Synthesized DNA Sequence
tatagagtattgtatagagagaaa

SEQ ID NO: 622 - DNA, 24 bp, Artificial Sequence
  Description of Artificial SequenceArtificially Synthesized DNA Sequence
aaatagtaagaatgtagttgttga

SEQ ID NO: 623 - DNA, 24 bp, Artificial Sequence
  Description of Artificial SequenceArtificially Synthesized DNA Sequence
tgagtgtgatttatgattaagtta

SEQ ID NO: 624 - DNA, 24 bp, Artificial Sequence
  Description of Artificial SequenceArtificially Synthesized DNA Sequence
agaatttgttgtagtgttatgatt

SEQ ID NO: 625 - DNA, 24 bp, Artificial Sequence
  Description of Artificial SequenceArtificially Synthesized DNA Sequence
gattgaagaaagaaatagtttgaa

SEQ ID NO: 626 - DNA, 24 bp, Artificial Sequence
  Description of Artificial SequenceArtificially Synthesized DNA Sequence
gataatagagaatagtagagttaa

SEQ ID NO: 627 - DNA, 24 bp, Artificial Sequence
  Description of Artificial SequenceArtificially Synthesized DNA Sequence
gattgaaatttgtagttatagtga

SEQ ID NO: 628 - DNA, 24 bp, Artificial Sequence
  Description of Artificial SequenceArtificially Synthesized DNA Sequence
gatttaagaagatgaataatgtag

SEQ ID NO: 629 - DNA, 24 bp, Artificial Sequence
  Description of Artificial SequenceArtificially Synthesized DNA Sequence
tttgagagaaagtagaataagata

SEQ ID NO: 630 - DNA, 24 bp, Artificial Sequence
  Description of Artificial SequenceArtificially Synthesized DNA Sequence
gattaagagtaaatgagtataaga

SEQ ID NO: 631 - DNA, 24 bp, Artificial Sequence
  Description of Artificial SequenceArtificially Synthesized DNA Sequence
tttgatagaattgaaatttgagag

SEQ ID NO: 632 - DNA, 24 bp, Artificial Sequence
  Description of Artificial SequenceArtificially Synthesized DNA Sequence
tgaagaagagtgttataagattta

SEQ ID NO: 633 - DNA, 24 bp, Artificial Sequence
  Description of Artificial SequenceArtificially Synthesized DNA Sequence
gtgaaatgatttagagtaataagt

SEQ ID NO: 634 - DNA, 24 bp, Artificial Sequence
  Description of Artificial SequenceArtificially Synthesized DNA Sequence
aaataagaatagagagagaaagtt

SEQ ID NO: 635 - DNA, 24 bp, Artificial Sequence
  Description of Artificial SequenceArtificially Synthesized DNA Sequence
gttgtaaagtaatagagaaattag

SEQ ID NO: 636 - DNA, 24 bp, Artificial Sequence
  Description of Artificial SequenceArtificially Synthesized DNA Sequence
agtgatttagattatgtgatgatt

SEQ ID NO: 637 - DNA, 24 bp, Artificial Sequence
  Description of Artificial SequenceArtificially Synthesized DNA Sequence
agagtatagtttagatttatgtag

SEQ ID NO: 638 - DNA, 24 bp, Artificial Sequence
  Description of Artificial SequenceArtificially Synthesized DNA Sequence
atgattagatagtgaaattgttag

SEQ ID NO: 639 - DNA, 24 bp, Artificial Sequence
  Description of Artificial SequenceArtificially Synthesized DNA Sequence
atgaaatgtattagtttagagttg

SEQ ID NO: 640 - DNA, 24 bp, Artificial Sequence
  Description of Artificial SequenceArtificially Synthesized DNA Sequence
atattgagtgagagttattgttaa

SEQ ID NO: 641 - DNA, 24 bp, Artificial Sequence
  Description of Artificial SequenceArtificially Synthesized DNA Sequence
agatgtgtattgaattaagaagtt

SEQ ID NO: 642 - DNA, 24 bp, Artificial Sequence
  Description of Artificial SequenceArtificially Synthesized DNA Sequence
taatgtgttgatagaatagagata

SEQ ID NO: 643 - DNA, 24 bp, Artificial Sequence
  Description of Artificial SequenceArtificially Synthesized DNA Sequence
aaattagttgaaagtatgagaaag

SEQ ID NO: 644 - DNA, 24 bp, Artificial Sequence
  Description of Artificial SequenceArtificially Synthesized DNA Sequence
tttagagttgaagaaatgttaatg

SEQ ID NO: 645 - DNA, 24 bp, Artificial Sequence
  Description of Artificial SequenceArtificially Synthesized DNA Sequence
gattgttgattattgatgaatttg

SEQ ID NO: 646 - DNA, 24 bp, Artificial Sequence
  Description of Artificial SequenceArtificially Synthesized DNA Sequence
tgttgttgttgaattgaagaatta

SEQ ID NO: 647 - DNA, 24 bp, Artificial Sequence
  Description of Artificial SequenceArtificially Synthesized DNA Sequence
attaagtaagaattgagagtttga

SEQ ID NO: 648 - DNA, 24 bp, Artificial Sequence
  Description of Artificial SequenceArtificially Synthesized DNA Sequence
gtatgttgtaatgtattaagaaag

SEQ ID NO: 649 - DNA, 24 bp, Artificial Sequence
  Description of Artificial SequenceArtificially Synthesized DNA Sequence
tagttgtgatttatgtaatgattg

SEQ ID NO: 650 - DNA, 24 bp, Artificial Sequence
  Description of Artificial SequenceArtificially Synthesized DNA Sequence
tgataatgaaagtttatagagaga

SEQ ID NO: 651 - DNA, 24 bp, Artificial Sequence
  Description of Artificial SequenceArtificially Synthesized DNA Sequence
gtaagattgtttgtatgataagat

SEQ ID NO: 652 - DNA, 24 bp, Artificial Sequence
  Description of Artificial SequenceArtificially Synthesized DNA Sequence
ttgaattaagagtaagatgtttag

SEQ ID NO: 653 - DNA, 24 bp, Artificial Sequence
  Description of Artificial SequenceArtificially Synthesized DNA Sequence
aagtgtttgtttagagtaaagata

SEQ ID NO: 654 - DNA, 24 bp, Artificial Sequence
  Description of Artificial SequenceArtificially Synthesized DNA Sequence
agagagataaagtatagaagttaa

SEQ ID NO: 655 - DNA, 24 bp, Artificial Sequence
  Description of Artificial SequenceArtificially Synthesized DNA Sequence
attatgaatagttagaaagagagt

SEQ ID NO: 656 - DNA, 24 bp, Artificial Sequence
  Description of Artificial SequenceArtificially Synthesized DNA Sequence
ttgttgatattagagaatgtgttt

SEQ ID NO: 657 - DNA, 24 bp, Artificial Sequence
  Description of Artificial SequenceArtificially Synthesized DNA Sequence
tttattgagagtttgttatttgtg

SEQ ID NO: 658 - DNA, 24 bp, Artificial Sequence
  Description of Artificial SequenceArtificially Synthesized DNA Sequence
agtgttaagaagttgattattgat

SEQ ID NO: 659 - DNA, 24 bp, Artificial Sequence
  Description of Artificial SequenceArtificially Synthesized DNA Sequence
gagaaatgattgaatgttgataat

SEQ ID NO: 660 - DNA, 24 bp, Artificial Sequence
  Description of Artificial SequenceArtificially Synthesized DNA Sequence
gataagtattagtatgagtgtaat

SEQ ID NO: 661 - DNA, 24 bp, Artificial Sequence
  Description of Artificial SequenceArtificially Synthesized DNA Sequence
tttgatttaagagtgttgaatgta

SEQ ID NO: 662 - DNA, 24 bp, Artificial Sequence
  Description of Artificial SequenceArtificially Synthesized DNA Sequence
aagttagtaaatagagtagaaaga

SEQ ID NO: 663 - DNA, 24 bp, Artificial Sequence
  Description of Artificial SequenceArtificially Synthesized DNA Sequence
gtaaagtatgaatatgtgaaatgt

SEQ ID NO: 664 - DNA, 24 bp, Artificial Sequence
  Description of Artificial SequenceArtificially Synthesized DNA Sequence
taataagtgtgttgtgaatgtaat

SEQ ID NO: 665 - DNA, 24 bp, Artificial Sequence
  Description of Artificial SequenceArtificially Synthesized DNA Sequence
aaagatttagagtagaaagagaat

SEQ ID NO: 666 - DNA, 24 bp, Artificial Sequence
  Description of Artificial SequenceArtificially Synthesized DNA Sequence
ttagtttgagttgaaatagtaaag

SEQ ID NO: 667 - DNA, 24 bp, Artificial Sequence
  Description of Artificial SequenceArtificially Synthesized DNA Sequence
taatagtatgagtaagattgaaag

SEQ ID NO: 668 - DNA, 24 bp, Artificial Sequence
  Description of Artificial SequenceArtificially Synthesized DNA Sequence
gaagattagattgatgttagttaa

SEQ ID NO: 669 - DNA, 24 bp, Artificial Sequence
  Description of Artificial SequenceArtificially Synthesized DNA Sequence
taaagagagaagttagtaatagaa

SEQ ID NO: 670 - DNA, 24 bp, Artificial Sequence
  Description of Artificial SequenceArtificially Synthesized DNA Sequence
taagtatgagaaatgatgtgttat

SEQ ID NO: 671 - DNA, 24 bp, Artificial Sequence
  Description of Artificial SequenceArtificially Synthesized DNA Sequence
gagtttgtttgttagttattgata

SEQ ID NO: 672 - DNA, 24 bp, Artificial Sequence
  Description of Artificial SequenceArtificially Synthesized DNA Sequence
aagtaaagaaatgttaagagtagt

SEQ ID NO: 673 - DNA, 24 bp, Artificial Sequence
  Description of Artificial SequenceArtificially Synthesized DNA Sequence
atgagaattgttgttgaaatgtaa

SEQ ID NO: 674 - DNA, 24 bp, Artificial Sequence
  Description of Artificial SequenceArtificially Synthesized DNA Sequence
ttagattagagtagtagaagaata

SEQ ID NO: 675 - DNA, 24 bp, Artificial Sequence
  Description of Artificial SequenceArtificially Synthesized DNA Sequence
tagtgatgaagaagttagaaatta

SEQ ID NO: 676 - DNA, 24 bp, Artificial Sequence
  Description of Artificial SequenceArtificially Synthesized DNA Sequence
taatgtagtaatgtgatgataagt

SEQ ID NO: 677 - DNA, 24 bp, Artificial Sequence
  Description of Artificial SequenceArtificially Synthesized DNA Sequence
ttgagaaagaataagtagtgtaaa

SEQ ID NO: 678 - DNA, 24 bp, Artificial Sequence
  Description of Artificial SequenceArtificially Synthesized DNA Sequence
taatgagtgagattatagattgtt

SEQ ID NO: 679 - DNA, 24 bp, Artificial Sequence
  Description of Artificial SequenceArtificially Synthesized DNA Sequence
gtataagaaatgtgtgtttgatta

SEQ ID NO: 680 - DNA, 24 bp, Artificial Sequence
  Description of Artificial SequenceArtificially Synthesized DNA Sequence
gtgaatgtgttaatgaagatatat

SEQ ID NO: 681 - DNA, 24 bp, Artificial Sequence
  Description of Artificial SequenceArtificially Synthesized DNA Sequence
gaaagttattagtagttaaagatg

SEQ ID NO: 682 - DNA, 24 bp, Artificial Sequence
  Description of Artificial SequenceArtificially Synthesized DNA Sequence
tagaattgtgtttgataagtgata

SEQ ID NO: 683 - DNA, 24 bp, Artificial Sequence
  Description of Artificial SequenceArtificially Synthesized DNA Sequence
tgatttagattgagagttaaatga

SEQ ID NO: 684 - DNA, 24 bp, Artificial Sequence
  Description of Artificial SequenceArtificially Synthesized DNA Sequence
attattgagtttgaatgttgatag

SEQ ID NO: 685 - DNA, 24 bp, Artificial Sequence
  Description of Artificial SequenceArtificially Synthesized DNA Sequence
atagtagttatgtttgatttagtg

SEQ ID NO: 686 - DNA, 24 bp, Artificial Sequence
  Description of Artificial SequenceArtificially Synthesized DNA Sequence
atagaagaagaataaagttagaga

SEQ ID NO: 687 - DNA, 24 bp, Artificial Sequence
  Description of Artificial SequenceArtificially Synthesized DNA Sequence
gatgttgaaagtaatgaatttgta

SEQ ID NO: 688 - DNA, 24 bp, Artificial Sequence
  Description of Artificial SequenceArtificially Synthesized DNA Sequence
gagattgatagtagaaatgataaa

SEQ ID NO: 689 - DNA, 24 bp, Artificial Sequence
  Description of Artificial SequenceArtificially Synthesized DNA Sequence
tgagagaataaagtatgaatttga

SEQ ID NO: 690 - DNA, 24 bp, Artificial Sequence
  Description of Artificial SequenceArtificially Synthesized DNA Sequence
tataaagatgatgtgaattagtag

SEQ ID NO: 691 - DNA, 24 bp, Artificial Sequence
  Description of Artificial SequenceArtificially Synthesized DNA Sequence
ttatgtaagaatgtttgagagaaa

SEQ ID NO: 692 - DNA, 24 bp, Artificial Sequence
  Description of Artificial SequenceArtificially Synthesized DNA Sequence
agtaaatgatgaatgatatgatga

SEQ ID NO: 693 - DNA, 24 bp, Artificial Sequence
  Description of Artificial SequenceArtificially Synthesized DNA Sequence
gaaatttgtgttaaagttgaatga

SEQ ID NO: 694 - DNA, 24 bp, Artificial Sequence
  Description of Artificial SequenceArtificially Synthesized DNA Sequence
gatgaatgattgtgtttaagtata

SEQ ID NO: 695 - DNA, 24 bp, Artificial Sequence
  Description of Artificial SequenceArtificially Synthesized DNA Sequence
gaaataagtgagagttaatgaaat

SEQ ID NO: 696 - DNA, 24 bp, Artificial Sequence
  Description of Artificial SequenceArtificially Synthesized DNA Sequence
tgttgaaatagttattagtttgtg

SEQ ID NO: 697 - DNA, 24 bp, Artificial Sequence
  Description of Artificial SequenceArtificially Synthesized DNA Sequence
tttgagagtatattgatatgagaa

SEQ ID NO: 698 - DNA, 24 bp, Artificial Sequence
  Description of Artificial SequenceArtificially Synthesized DNA Sequence
attgtgtgtaaagtaagatttaag

SEQ ID NO: 699 - DNA, 24 bp, Artificial Sequence
  Description of Artificial SequenceArtificially Synthesized DNA Sequence
tatagtttgaagtgtgatgtattt

SEQ ID NO: 700 - DNA, 24 bp, Artificial Sequence
  Description of Artificial SequenceArtificially Synthesized DNA Sequence
gtgaagttatagtgtataaagaat

SEQ ID NO: 701 - DNA, 24 bp, Artificial Sequence
  Description of Artificial SequenceArtificially Synthesized DNA Sequence
gtatgttgaatagtaaatagattg

SEQ ID NO: 702 - DNA, 24 bp, Artificial Sequence
  Description of Artificial SequenceArtificially Synthesized DNA Sequence
ttagaaagtgtgatttgtgtattt

SEQ ID NO: 703 - DNA, 24 bp, Artificial Sequence
  Description of Artificial SequenceArtificially Synthesized DNA Sequence
tttagtaatatgtaagagatgtga

SEQ ID NO: 704 - DNA, 24 bp, Artificial Sequence
  Description of Artificial SequenceArtificially Synthesized DNA Sequence
agtatgtatagatgatgtttgttt

SEQ ID NO: 705 - DNA, 24 bp, Artificial Sequence
  Description of Artificial SequenceArtificially Synthesized DNA Sequence
atttaagtaaagtgtagagataag

SEQ ID NO: 706 - DNA, 24 bp, Artificial Sequence
  Description of Artificial SequenceArtificially Synthesized DNA Sequence
atttgtgttgaattgtaaagtgaa

SEQ ID NO: 707 - DNA, 24 bp, Artificial Sequence
  Description of Artificial SequenceArtificially Synthesized DNA Sequence
atgttattagattgtgatgaatga

SEQ ID NO: 708 - DNA, 24 bp, Artificial Sequence
  Description of Artificial SequenceArtificially Synthesized DNA Sequence
tagtagtagaatatgaaattagag

SEQ ID NO: 709 - DNA, 24 bp, Artificial Sequence
  Description of Artificial SequenceArtificially Synthesized DNA Sequence
tttaatgagaagagttagagtata

SEQ ID NO: 710 - DNA, 24 bp, Artificial Sequence
  Description of Artificial SequenceArtificially Synthesized DNA Sequence
aaagtttagtagagtgtatgtaaa

SEQ ID NO: 711 - DNA, 24 bp, Artificial Sequence
  Description of Artificial SequenceArtificially Synthesized DNA Sequence
atatatgatagtagagtagattag

SEQ ID NO: 712 - DNA, 24 bp, Artificial Sequence
  Description of Artificial SequenceArtificially Synthesized DNA Sequence
tgagaagttaattgtatagattga

SEQ ID NO: 713 - DNA, 24 bp, Artificial Sequence
  Description of Artificial SequenceArtificially Synthesized DNA Sequence
tatagagatgttatatgaagttgt

SEQ ID NO: 714 - DNA, 24 bp, Artificial Sequence
  Description of Artificial SequenceArtificially Synthesized DNA Sequence
aaatttgttaagttgttgttgttg

SEQ ID NO: 715 - DNA, 24 bp, Artificial Sequence
  Description of Artificial SequenceArtificially Synthesized DNA Sequence
ttgttgaagatgaaagtagaatta

SEQ ID NO: 716 - DNA, 24 bp, Artificial Sequence
  Description of Artificial SequenceArtificially Synthesized DNA Sequence
aagagataagtagtgtttatgttt

SEQ ID NO: 717 - DNA, 24 bp, Artificial Sequence
  Description of Artificial SequenceArtificially Synthesized DNA Sequence
aataagaagaagtgaaagattgat

SEQ ID NO: 718 - DNA, 24 bp, Artificial Sequence
  Description of Artificial SequenceArtificially Synthesized DNA Sequence
taagttaaagttgatgattgatag

SEQ ID NO: 719 - DNA, 24 bp, Artificial Sequence
  Description of Artificial SequenceArtificially Synthesized DNA Sequence
atataagataagagtgtaagtgat

SEQ ID NO: 720 - DNA, 24 bp, Artificial Sequence
  Description of Artificial SequenceArtificially Synthesized DNA Sequence
gttaaatgttgttgtttaagtgat

SEQ ID NO: 721 - DNA, 24 bp, Artificial Sequence
  Description of Artificial SequenceArtificially Synthesized DNA Sequence
gagttaagttattagttaagaagt

SEQ ID NO: 722 - DNA, 24 bp, Artificial Sequence
  Description of Artificial SequenceArtificially Synthesized DNA Sequence
tattagagtttgagaataagtagt

SEQ ID NO: 723 - DNA, 24 bp, Artificial Sequence
  Description of Artificial SequenceArtificially Synthesized DNA Sequence
taatgttgttatgtgttagatgtt

SEQ ID NO: 724 - DNA, 24 bp, Artificial Sequence
  Description of Artificial SequenceArtificially Synthesized DNA Sequence
gaaagttgatagaatgtaatgttt

SEQ ID NO: 725 - DNA, 24 bp, Artificial Sequence
  Description of Artificial SequenceArtificially Synthesized DNA Sequence
tgatagatgaattgattgattagt

SEQ ID NO: 726 - DNA, 24 bp, Artificial Sequence
  Description of Artificial SequenceArtificially Synthesized DNA Sequence
atgatagagtaaagaataagttgt

SEQ ID NO: 727 - DNA, 24 bp, Artificial Sequence
  Description of Artificial SequenceArtificially Synthesized DNA Sequence
agtaagtgttagatagtattgaat

SEQ ID NO: 728 - DNA, 24 bp, Artificial Sequence
  Description of Artificial SequenceArtificially Synthesized DNA Sequence
atgtagattaaagtagtgtatgtt

SEQ ID NO: 729 - DNA, 24 bp, Artificial Sequence
  Description of Artificial SequenceArtificially Synthesized DNA Sequence
ttattgataatgagagagttaaag

SEQ ID NO: 730 - DNA, 24 bp, Artificial Sequence
  Description of Artificial SequenceArtificially Synthesized DNA Sequence
atttgttatgataaatgtgtagtg

SEQ ID NO: 731 - DNA, 24 bp, Artificial Sequence
  Description of Artificial SequenceArtificially Synthesized DNA Sequence
ttgaagaaataagagtaataagag

SEQ ID NO: 732 - DNA, 24 bp, Artificial Sequence
  Description of Artificial SequenceArtificially Synthesized DNA Sequence
tgtgtaataagtagtaagattaga

SEQ ID NO: 733 - DNA, 24 bp, Artificial Sequence
  Description of Artificial SequenceArtificially Synthesized DNA Sequence
atgaaagttagagtttatgataag

SEQ ID NO: 734 - DNA, 24 bp, Artificial Sequence
  Description of Artificial SequenceArtificially Synthesized DNA Sequence
attagttaagagagtttgtagatt

SEQ ID NO: 735 - DNA, 24 bp, Artificial Sequence
  Description of Artificial SequenceArtificially Synthesized DNA Sequence
tgtagtattgtatgattaaagtgt

SEQ ID NO: 736 - DNA, 24 bp, Artificial Sequence
  Description of Artificial SequenceArtificially Synthesized DNA Sequence
agttgataaagaagaagagtatat

SEQ ID NO: 737 - DNA, 24 bp, Artificial Sequence
  Description of Artificial SequenceArtificially Synthesized DNA Sequence
gtaatgagataaagagagataatt

SEQ ID NO: 738 - DNA, 24 bp, Artificial Sequence
  Description of Artificial SequenceArtificially Synthesized DNA Sequence
tgtgttgaagataaagtttatgat

SEQ ID NO: 739 - DNA, 24 bp, Artificial Sequence
  Description of Artificial SequenceArtificially Synthesized DNA Sequence
aagaagagtagttagaattgatta

SEQ ID NO: 740 - DNA, 24 bp, Artificial Sequence
  Description of Artificial SequenceArtificially Synthesized DNA Sequence
gaatgaagatgaagtttgttaata

SEQ ID NO: 741 - DNA, 24 bp, Artificial Sequence
  Description of Artificial SequenceArtificially Synthesized DNA Sequence
aaattgttgagataagatagtgat

SEQ ID NO: 742 - DNA, 24 bp, Artificial Sequence
  Description of Artificial SequenceArtificially Synthesized DNA Sequence
tgattgtttaatgatgtgtgatta

SEQ ID NO: 743 - DNA, 24 bp, Artificial Sequence
  Description of Artificial SequenceArtificially Synthesized DNA Sequence
atgaagtattgttgagtgatttaa

SEQ ID NO: 744 - DNA, 24 bp, Artificial Sequence
  Description of Artificial SequenceArtificially Synthesized DNA Sequence
gtgtaaatgtttgagatgtatatt

SEQ ID NO: 745 - DNA, 24 bp, Artificial Sequence
  Description of Artificial SequenceArtificially Synthesized DNA Sequence
aattgatgagtttaaagagttgat

SEQ ID NO: 746 - DNA, 24 bp, Artificial Sequence
  Description of Artificial SequenceArtificially Synthesized DNA Sequence
tttgtgtaatatgattgagagttt

SEQ ID NO: 747 - DNA, 24 bp, Artificial Sequence
  Description of Artificial SequenceArtificially Synthesized DNA Sequence
gtagtagatgattaagaagataaa

SEQ ID NO: 748 - DNA, 24 bp, Artificial Sequence
  Description of Artificial SequenceArtificially Synthesized DNA Sequence
tttaatgtgaaatttgttgtgagt

SEQ ID NO: 749 - DNA, 24 bp, Artificial Sequence
  Description of Artificial SequenceArtificially Synthesized DNA Sequence
gtaaagaattagataaagagtgat

SEQ ID NO: 750 - DNA, 24 bp, Artificial Sequence
  Description of Artificial SequenceArtificially Synthesized DNA Sequence
aatagttaagtttaagagttgtgt

SEQ ID NO: 751 - DNA, 24 bp, Artificial Sequence
  Description of Artificial SequenceArtificially Synthesized DNA Sequence
gtgtgatgtttatagatttgttat

SEQ ID NO: 752 - DNA, 24 bp, Artificial Sequence
  Description of Artificial SequenceArtificially Synthesized DNA Sequence
gtatagtgtgattagatttgtaaa

SEQ ID NO: 753 - DNA, 24 bp, Artificial Sequence
  Description of Artificial SequenceArtificially Synthesized DNA Sequence
gttgtaagaaagatatgtaagaaa

SEQ ID NO: 754 - DNA, 24 bp, Artificial Sequence
  Description of Artificial SequenceArtificially Synthesized DNA Sequence
atattagattgtaaagagagtgaa

SEQ ID NO: 755 - DNA, 24 bp, Artificial Sequence
  Description of Artificial SequenceArtificially Synthesized DNA Sequence
gagtgatattgaaattagattgta

SEQ ID NO: 756 - DNA, 24 bp, Artificial Sequence
  Description of Artificial SequenceArtificially Synthesized DNA Sequence
taagaagttaaagaagagagttta

SEQ ID NO: 757 - DNA, 24 bp, Artificial Sequence
  Description of Artificial SequenceArtificially Synthesized DNA Sequence
gatgttagataaagtttaagtagt

SEQ ID NO: 758 - DNA, 24 bp, Artificial Sequence
  Description of Artificial SequenceArtificially Synthesized DNA Sequence
gtgattgtatgagaaatgttaaat

SEQ ID NO: 759 - DNA, 24 bp, Artificial Sequence
  Description of Artificial SequenceArtificially Synthesized DNA Sequence
tgattattgtaagaaagattgaga

SEQ ID NO: 760 - DNA, 24 bp, Artificial Sequence
  Description of Artificial SequenceArtificially Synthesized DNA Sequence
aagaattgtgtaagtttatgagta

SEQ ID NO: 761 - DNA, 24 bp, Artificial Sequence
  Description of Artificial SequenceArtificially Synthesized DNA Sequence
ttgtatttagaagatttgtagatg

SEQ ID NO: 762 - DNA, 24 bp, Artificial Sequence
  Description of Artificial SequenceArtificially Synthesized DNA Sequence
tatatgtttgtgtaagaagaaatg

SEQ ID NO: 763 - DNA, 24 bp, Artificial Sequence
  Description of Artificial SequenceArtificially Synthesized DNA Sequence
gataatgtgtgaatttgtgaataa

SEQ ID NO: 764 - DNA, 24 bp, Artificial Sequence
  Description of Artificial SequenceArtificially Synthesized DNA Sequence
ttagaaatgtgagatttaagagtt

SEQ ID NO: 765 - DNA, 24 bp, Artificial Sequence
  Description of Artificial SequenceArtificially Synthesized DNA Sequence
agtgtagaatttgtatttagttgt

SEQ ID NO: 766 - DNA, 24 bp, Artificial Sequence
  Description of Artificial SequenceArtificially Synthesized DNA Sequence
tagttaagatagagtaaatgatag

SEQ ID NO: 767 - DNA, 24 bp, Artificial Sequence
  Description of Artificial SequenceArtificially Synthesized DNA Sequence
gaagtgatattgtaaattgataag

SEQ ID NO: 768 - DNA, 24 bp, Artificial Sequence
  Description of Artificial SequenceArtificially Synthesized DNA Sequence
gtaattgtgttagatttaagaagt

SEQ ID NO: 769 - DNA, 24 bp, Artificial Sequence
  Description of Artificial SequenceArtificially Synthesized DNA Sequence
tgatatttgtgaattgatagtatg

SEQ ID NO: 770 - DNA, 24 bp, Artificial Sequence
  Description of Artificial SequenceArtificially Synthesized DNA Sequence
aagtaaagagatatagttaagttg

SEQ ID NO: 771 - DNA, 24 bp, Artificial Sequence
  Description of Artificial SequenceArtificially Synthesized DNA Sequence
attagttaagttatttgtgagtga

SEQ ID NO: 772 - DNA, 24 bp, Artificial Sequence
  Description of Artificial SequenceArtificially Synthesized DNA Sequence
agatgaagtagtttatgaattaga

SEQ ID NO: 773 - DNA, 24 bp, Artificial Sequence
  Description of Artificial SequenceArtificially Synthesized DNA Sequence
tgagttagttaagtgatagttaaa

SEQ ID NO: 774 - DNA, 24 bp, Artificial Sequence
  Description of Artificial SequenceArtificially Synthesized DNA Sequence
ttattgtagatttagagaagatga

SEQ ID NO: 775 - DNA, 24 bp, Artificial Sequence
  Description of Artificial SequenceArtificially Synthesized DNA Sequence
tatttgtgtttgttgattagatag

SEQ ID NO: 776 - DNA, 24 bp, Artificial Sequence
  Description of Artificial SequenceArtificially Synthesized DNA Sequence
gtataatgtgtgtgaaagttataa

SEQ ID NO: 777 - DNA, 24 bp, Artificial Sequence
  Description of Artificial SequenceArtificially Synthesized DNA Sequence
tatatgttgagtataaagagagaa

SEQ ID NO: 778 - DNA, 24 bp, Artificial Sequence
  Description of Artificial SequenceArtificially Synthesized DNA Sequence
ttagttagtttaaagattgtgagt

SEQ ID NO: 779 - DNA, 24 bp, Artificial Sequence
  Description of Artificial SequenceArtificially Synthesized DNA Sequence
tttagaataagtgatgtgatgaaa

SEQ ID NO: 780 - DNA, 24 bp, Artificial Sequence
  Description of Artificial SequenceArtificially Synthesized DNA Sequence
agagtaatgtgtaaatagttagat

SEQ ID NO: 781 - DNA, 24 bp, Artificial Sequence
  Description of Artificial SequenceArtificially Synthesized DNA Sequence
tgtgataaagagaaattagttgtt

SEQ ID NO: 782 - DNA, 24 bp, Artificial Sequence
  Description of Artificial SequenceArtificially Synthesized DNA Sequence
gaatttagtgaatgtttgagatta

SEQ ID NO: 783 - DNA, 24 bp, Artificial Sequence
  Description of Artificial SequenceArtificially Synthesized DNA Sequence
tgtgatgtgtaagtatatgaaatt

SEQ ID NO: 784 - DNA, 24 bp, Artificial Sequence
  Description of Artificial SequenceArtificially Synthesized DNA Sequence
ttgtgaatgattaatgaatagaag

SEQ ID NO: 785 - DNA, 24 bp, Artificial Sequence
  Description of Artificial SequenceArtificially Synthesized DNA Sequence
aatgttgtttagattgagaaagtt

SEQ ID NO: 786 - DNA, 24 bp, Artificial Sequence
  Description of Artificial SequenceArtificially Synthesized DNA Sequence
agattgtgttagtattagtataag

SEQ ID NO: 787 - DNA, 24 bp, Artificial Sequence
  Description of Artificial SequenceArtificially Synthesized DNA Sequence
ttgatgtattagaaagtttatgtg

SEQ ID NO: 788 - DNA, 24 bp, Artificial Sequence
  Description of Artificial SequenceArtificially Synthesized DNA Sequence
tatgattgtgtgttagagaattta

SEQ ID NO: 789 - DNA, 24 bp, Artificial Sequence
  Description of Artificial SequenceArtificially Synthesized DNA Sequence
tagtgtagatatttgatagttatg

SEQ ID NO: 790 - DNA, 24 bp, Artificial Sequence
  Description of Artificial SequenceArtificially Synthesized DNA Sequence
agtttaatgtgtttagttgttatg

SEQ ID NO: 791 - DNA, 24 bp, Artificial Sequence
  Description of Artificial SequenceArtificially Synthesized DNA Sequence
tgtgtaaagtagaaagtaaagatt

SEQ ID NO: 792 - DNA, 24 bp, Artificial Sequence
  Description of Artificial SequenceArtificially Synthesized DNA Sequence
gttatgatatagtgagttgttatt

SEQ ID NO: 793 - DNA, 24 bp, Artificial Sequence
  Description of Artificial SequenceArtificially Synthesized DNA Sequence
tttgattgaatgttaatagtgtgt

SEQ ID NO: 794 - DNA, 24 bp, Artificial Sequence
  Description of Artificial SequenceArtificially Synthesized DNA Sequence
agagtattagtagttattgtaagt

SEQ ID NO: 795 - DNA, 24 bp, Artificial Sequence
  Description of Artificial SequenceArtificially Synthesized DNA Sequence
taagtagaaagaagaagatatttg

SEQ ID NO: 796 - DNA, 24 bp, Artificial Sequence
  Description of Artificial SequenceArtificially Synthesized DNA Sequence
agaaagagaattatgtaatgaaag

SEQ ID NO: 797 - DNA, 24 bp, Artificial Sequence
  Description of Artificial SequenceArtificially Synthesized DNA Sequence
ttagatttgttagtgtgatttaag

SEQ ID NO: 798 - DNA, 24 bp, Artificial Sequence
  Description of Artificial SequenceArtificially Synthesized DNA Sequence
gatgattaagatatagagatagtt

SEQ ID NO: 799 - DNA, 24 bp, Artificial Sequence
  Description of Artificial SequenceArtificially Synthesized DNA Sequence
atatttgagtgattaagagtaatg

SEQ ID NO: 800 - DNA, 24 bp, Artificial Sequence
  Description of Artificial SequenceArtificially Synthesized DNA Sequence
tgtattgtgagttaagtataagtt

SEQ ID NO: 801 - DNA, 24 bp, Artificial Sequence
  Description of Artificial SequenceArtificially Synthesized DNA Sequence
aatttagtagaaagtgttgtgttt

SEQ ID NO: 802 - DNA, 24 bp, Artificial Sequence
  Description of Artificial SequenceArtificially Synthesized DNA Sequence
gttagaagattaagttgaataatg

SEQ ID NO: 803 - DNA, 24 bp, Artificial Sequence
  Description of Artificial SequenceArtificially Synthesized DNA Sequence
taaagtatgtgagatgatttatgt

SEQ ID NO: 804 - DNA, 24 bp, Artificial Sequence
  Description of Artificial SequenceArtificially Synthesized DNA Sequence
tgaaatgattaaagatgaagatga

SEQ ID NO: 805 - DNA, 24 bp, Artificial Sequence
  Description of Artificial SequenceArtificially Synthesized DNA Sequence
ttattagatgttgagtgtttgttt

SEQ ID NO: 806 - DNA, 24 bp, Artificial Sequence
  Description of Artificial SequenceArtificially Synthesized DNA Sequence
tagtgtttaaagagtagtatatga

SEQ ID NO: 807 - DNA, 24 bp, Artificial Sequence
  Description of Artificial SequenceArtificially Synthesized DNA Sequence
agttataagtaaatgatgttgatg

SEQ ID NO: 808 - DNA, 24 bp, Artificial Sequence
  Description of Artificial SequenceArtificially Synthesized DNA Sequence
ttaagagagaaataagtgtattgt

SEQ ID NO: 809 - DNA, 24 bp, Artificial Sequence
  Description of Artificial SequenceArtificially Synthesized DNA Sequence
gatattgaaatgtgtaaatgatga

SEQ ID NO: 810 - DNA, 24 bp, Artificial Sequence
  Description of Artificial SequenceArtificially Synthesized DNA Sequence
atgatgaattaagaaagaaagaga

SEQ ID NO: 811 - DNA, 24 bp, Artificial Sequence
  Description of Artificial SequenceArtificially Synthesized DNA Sequence
gaatagtttgatttgtgtttgtta

SEQ ID NO: 812 - DNA, 24 bp, Artificial Sequence
  Description of Artificial SequenceArtificially Synthesized DNA Sequence
agttgtttagatttgatttgtaag

SEQ ID NO: 813 - DNA, 24 bp, Artificial Sequence
  Description of Artificial SequenceArtificially Synthesized DNA Sequence
gtatgagatttgatataagattag

SEQ ID NO: 814 - DNA, 24 bp, Artificial Sequence
  Description of Artificial SequenceArtificially Synthesized DNA Sequence
tttatagtgagtatagtgatgatt

SEQ ID NO: 815 - DNA, 24 bp, Artificial Sequence
  Description of Artificial SequenceArtificially Synthesized DNA Sequence
tatatgtgaagatataagtgtttg

SEQ ID NO: 816 - DNA, 24 bp, Artificial Sequence
  Description of Artificial SequenceArtificially Synthesized DNA Sequence
attgatagatgatagtaattgagt

SEQ ID NO: 817 - DNA, 24 bp, Artificial Sequence
  Description of Artificial SequenceArtificially Synthesized DNA Sequence
tgatagatgtgaagaatttgattt

SEQ ID NO: 818 - DNA, 24 bp, Artificial Sequence
  Description of Artificial SequenceArtificially Synthesized DNA Sequence
gaagatattgaaagaatttgatgt

SEQ ID NO: 819 - DNA, 24 bp, Artificial Sequence
  Description of Artificial SequenceArtificially Synthesized DNA Sequence
gatgtttagtgtagatatagattt

SEQ ID NO: 820 - DNA, 24 bp, Artificial Sequence
  Description of Artificial SequenceArtificially Synthesized DNA Sequence
gaatattgagttataagtagtagt

SEQ ID NO: 821 - DNA, 24 bp, Artificial Sequence
  Description of Artificial SequenceArtificially Synthesized DNA Sequence
agtgagtaagtaatagaaagattt

SEQ ID NO: 822 - DNA, 24 bp, Artificial Sequence
  Description of Artificial SequenceArtificially Synthesized DNA Sequence
gtagaataagtaatttgtgagata

SEQ ID NO: 823 - DNA, 24 bp, Artificial Sequence
  Description of Artificial SequenceArtificially Synthesized DNA Sequence
gagttatttgagatttagatgttt

SEQ ID NO: 824 - DNA, 24 bp, Artificial Sequence
  Description of Artificial SequenceArtificially Synthesized DNA Sequence
gaaatgatgattgaatttagagat

SEQ ID NO: 825 - DNA, 24 bp, Artificial Sequence
  Description of Artificial SequenceArtificially Synthesized DNA Sequence
aaatagtgtgagaatagttaagta

SEQ ID NO: 826 - DNA, 24 bp, Artificial Sequence
  Description of Artificial SequenceArtificially Synthesized DNA Sequence
atgtgttaagttgtagaagaataa

SEQ ID NO: 827 - DNA, 24 bp, Artificial Sequence
  Description of Artificial SequenceArtificially Synthesized DNA Sequence
ataatgagttaatagtgtaagaag

SEQ ID NO: 828 - DNA, 24 bp, Artificial Sequence
  Description of Artificial SequenceArtificially Synthesized DNA Sequence
ataagagatgtttaagttagaaag

SEQ ID NO: 829 - DNA, 24 bp, Artificial Sequence
  Description of Artificial SequenceArtificially Synthesized DNA Sequence
tgttagtgttagaaatatgaaaga

SEQ ID NO: 830 - DNA, 24 bp, Artificial Sequence
  Description of Artificial SequenceArtificially Synthesized DNA Sequence
tttagaagattgttagataagttg

SEQ ID NO: 831 - DNA, 24 bp, Artificial Sequence
  Description of Artificial SequenceArtificially Synthesized DNA Sequence
gtgtaatgtataagatagttaagt

SEQ ID NO: 832 - DNA, 24 bp, Artificial Sequence
  Description of Artificial SequenceArtificially Synthesized DNA Sequence
tattagagagaaattgtagagatt

SEQ ID NO: 833 - DNA, 24 bp, Artificial Sequence
  Description of Artificial SequenceArtificially Synthesized DNA Sequence
tagtgagataaagtaaagtttatg

SEQ ID NO: 834 - DNA, 24 bp, Artificial Sequence
  Description of Artificial SequenceArtificially Synthesized DNA Sequence
ttgtgaaagttaagtaagttagtt

SEQ ID NO: 835 - DNA, 24 bp, Artificial Sequence
  Description of Artificial SequenceArtificially Synthesized DNA Sequence
aaagtgtaagttgaagaatattga

SEQ ID NO: 836 - DNA, 24 bp, Artificial Sequence
  Description of Artificial SequenceArtificially Synthesized DNA Sequence
gaatagagtgttatttgaaataga

SEQ ID NO: 837 - DNA, 24 bp, Artificial Sequence
  Description of Artificial SequenceArtificially Synthesized DNA Sequence
tataagagagagataagtaataag

SEQ ID NO: 838 - DNA, 24 bp, Artificial Sequence
  Description of Artificial SequenceArtificially Synthesized DNA Sequence
tgagtgaaattgatagagtaaatt

SEQ ID NO: 839 - DNA, 24 bp, Artificial Sequence
  Description of Artificial SequenceArtificially Synthesized DNA Sequence
gatgaataagtttaagtgagaaat

SEQ ID NO: 840 - DNA, 24 bp, Artificial Sequence
  Description of Artificial SequenceArtificially Synthesized DNA Sequence
gtgtgatatgtttattgattaagt

SEQ ID NO: 841 - DNA, 24 bp, Artificial Sequence
  Description of Artificial SequenceArtificially Synthesized DNA Sequence
taaagtgagtgtaaatgataatga

SEQ ID NO: 842 - DNA, 24 bp, Artificial Sequence
  Description of Artificial SequenceArtificially Synthesized DNA Sequence
gtagagtttgatttgaaagaatat

SEQ ID NO: 843 - DNA, 24 bp, Artificial Sequence
  Description of Artificial SequenceArtificially Synthesized DNA Sequence
gaatattgttatgtttgttatgag

SEQ ID NO: 844 - DNA, 24 bp, Artificial Sequence
  Description of Artificial SequenceArtificially Synthesized DNA Sequence
gtgtaataagatgtattgttgttt

SEQ ID NO: 845 - DNA, 24 bp, Artificial Sequence
  Description of Artificial SequenceArtificially Synthesized DNA Sequence
taaattgattgtgagttgaagaat

SEQ ID NO: 846 - DNA, 24 bp, Artificial Sequence
  Description of Artificial SequenceArtificially Synthesized DNA Sequence
tgagatagttatagttaagtttag

SEQ ID NO: 847 - DNA, 24 bp, Artificial Sequence
  Description of Artificial SequenceArtificially Synthesized DNA Sequence
agtttgttaagattatgtagaaag

SEQ ID NO: 848 - DNA, 24 bp, Artificial Sequence
  Description of Artificial SequenceArtificially Synthesized DNA Sequence
gaatgtgtagaataagagattaaa

SEQ ID NO: 849 - DNA, 24 bp, Artificial Sequence
  Description of Artificial SequenceArtificially Synthesized DNA Sequence
gtattatgaaagaagttgttgttt

SEQ ID NO: 850 - DNA, 24 bp, Artificial Sequence
  Description of Artificial SequenceArtificially Synthesized DNA Sequence
gtgttatagaagttaaatgttaag

SEQ ID NO: 851 - DNA, 24 bp, Artificial Sequence
  Description of Artificial SequenceArtificially Synthesized DNA Sequence
ttaagagtagtgaatatgatagta

SEQ ID NO: 852 - DNA, 24 bp, Artificial Sequence
  Description of Artificial SequenceArtificially Synthesized DNA Sequence
aatgttataagatgagagtttagt

SEQ ID NO: 853 - DNA, 24 bp, Artificial Sequence
  Description of Artificial SequenceArtificially Synthesized DNA Sequence
atataagatttgatgtagtgtagt

SEQ ID NO: 854 - DNA, 24 bp, Artificial Sequence
  Description of Artificial SequenceArtificially Synthesized DNA Sequence
tatgtttgttgttgttaagtttga

SEQ ID NO: 855 - DNA, 24 bp, Artificial Sequence
  Description of Artificial SequenceArtificially Synthesized DNA Sequence
gatagtttagtatagaagataaag

SEQ ID NO: 856 - DNA, 24 bp, Artificial Sequence
  Description of Artificial SequenceArtificially Synthesized DNA Sequence
gttgaatatagagatagtaaatag

SEQ ID NO: 857 - DNA, 24 bp, Artificial Sequence
  Description of Artificial SequenceArtificially Synthesized DNA Sequence
agagaagatttagtaagaatgata

SEQ ID NO: 858 - DNA, 24 bp, Artificial Sequence
  Description of Artificial SequenceArtificially Synthesized DNA Sequence
tgaatgagaaagatattgagtatt

SEQ ID NO: 859 - DNA, 24 bp, Artificial Sequence
  Description of Artificial SequenceArtificially Synthesized DNA Sequence
tgaagattatagtagttgtataga

SEQ ID NO: 860 - DNA, 24 bp, Artificial Sequence
  Description of Artificial SequenceArtificially Synthesized DNA Sequence
gattagtagtattgaagattatgt

SEQ ID NO: 861 - DNA, 24 bp, Artificial Sequence
  Description of Artificial SequenceArtificially Synthesized DNA Sequence
tgaaatgtgtatttgtatgtttag

SEQ ID NO: 862 - DNA, 24 bp, Artificial Sequence
  Description of Artificial SequenceArtificially Synthesized DNA Sequence
attaaagttgatatgaaagaagtg

SEQ ID NO: 863 - DNA, 24 bp, Artificial Sequence
  Description of Artificial SequenceArtificially Synthesized DNA Sequence
aatgtagagattgtagtgaatatt

SEQ ID NO: 864 - DNA, 24 bp, Artificial Sequence
  Description of Artificial SequenceArtificially Synthesized DNA Sequence
ttatttgttgagtgtaaatgtgat

SEQ ID NO: 865 - DNA, 24 bp, Artificial Sequence
  Description of Artificial SequenceArtificially Synthesized DNA Sequence
atgtaattgtgaataatgtatgtg

SEQ ID NO: 866 - DNA, 24 bp, Artificial Sequence
  Description of Artificial SequenceArtificially Synthesized DNA Sequence
gatttgtatagagattagtaagta

SEQ ID NO: 867 - DNA, 24 bp, Artificial Sequence
  Description of Artificial SequenceArtificially Synthesized DNA Sequence
aatattgttgtttagagaaagaag

SEQ ID NO: 868 - DNA, 24 bp, Artificial Sequence
  Description of Artificial SequenceArtificially Synthesized DNA Sequence
atgatgatgtatttgtaaagagta

SEQ ID NO: 869 - DNA, 24 bp, Artificial Sequence
  Description of Artificial SequenceArtificially Synthesized DNA Sequence
aatgtatttgtgtgattgtgtaaa

SEQ ID NO: 870 - DNA, 24 bp, Artificial Sequence
  Description of Artificial SequenceArtificially Synthesized DNA Sequence
agtgttatgaagaatagtaagaat

SEQ ID NO: 871 - DNA, 24 bp, Artificial Sequence
  Description of Artificial SequenceArtificially Synthesized DNA Sequence
gttatgtagagatgaaagaaatta

SEQ ID NO: 872 - DNA, 24 bp, Artificial Sequence
  Description of Artificial SequenceArtificially Synthesized DNA Sequence
gtttgtattagataaatgagttgt

SEQ ID NO: 873 - DNA, 24 bp, Artificial Sequence
  Description of Artificial SequenceArtificially Synthesized DNA Sequence
tgatttatgagattaagagaaaga

SEQ ID NO: 874 - DNA, 24 bp, Artificial Sequence
  Description of Artificial SequenceArtificially Synthesized DNA Sequence
tttgtgtgttattgtaattgagat

SEQ ID NO: 875 - DNA, 24 bp, Artificial Sequence
  Description of Artificial SequenceArtificially Synthesized DNA Sequence
gatgtgtgatatgattaaagaaat

SEQ ID NO: 876 - DNA, 24 bp, Artificial Sequence
  Description of Artificial SequenceArtificially Synthesized DNA Sequence
agattatagatttgtagagaaagt

SEQ ID NO: 877 - DNA, 24 bp, Artificial Sequence
  Description of Artificial SequenceArtificially Synthesized DNA Sequence
gaagagtatgtaatagtattgtat

SEQ ID NO: 878 - DNA, 24 bp, Artificial Sequence
  Description of Artificial SequenceArtificially Synthesized DNA Sequence
tttgtaatgttgttgagtttaaga

SEQ ID NO: 879 - DNA, 24 bp, Artificial Sequence
  Description of Artificial SequenceArtificially Synthesized DNA Sequence
agtaaatagtagtatgaataagag

SEQ ID NO: 880 - DNA, 24 bp, Artificial Sequence
  Description of Artificial SequenceArtificially Synthesized DNA Sequence
gaatgttgaattgaaatatgagtt

SEQ ID NO: 881 - DNA, 24 bp, Artificial Sequence
  Description of Artificial SequenceArtificially Synthesized DNA Sequence
agtagttaattgatagtaagtttg

SEQ ID NO: 882 - DNA, 24 bp, Artificial Sequence
  Description of Artificial SequenceArtificially Synthesized DNA Sequence
agtgtaaagaaatgaatgaataag

SEQ ID NO: 883 - DNA, 24 bp, Artificial Sequence
  Description of Artificial SequenceArtificially Synthesized DNA Sequence
tgttagatatttgtgaaatgtgaa

SEQ ID NO: 884 - DNA, 24 bp, Artificial Sequence
  Description of Artificial SequenceArtificially Synthesized DNA Sequence
tgtatgttgagtttgaattgttat

SEQ ID NO: 885 - DNA, 24 bp, Artificial Sequence
  Description of Artificial SequenceArtificially Synthesized DNA Sequence
tgagtgaattagttatgttgttat

SEQ ID NO: 886 - DNA, 24 bp, Artificial Sequence
  Description of Artificial SequenceArtificially Synthesized DNA Sequence
gaagaaagaaatgagaaagattat

SEQ ID NO: 887 - DNA, 24 bp, Artificial Sequence
  Description of Artificial SequenceArtificially Synthesized DNA Sequence
ttaagtaagttgtgttgatattag

SEQ ID NO: 888 - DNA, 24 bp, Artificial Sequence
  Description of Artificial SequenceArtificially Synthesized DNA Sequence
atgatgtgtttgatttgaattgaa

SEQ ID NO: 889 - DNA, 24 bp, Artificial Sequence
  Description of Artificial SequenceArtificially Synthesized DNA Sequence
aagtaagtgaaattgttgtttgaa

SEQ ID NO: 890 - DNA, 24 bp, Artificial Sequence
  Description of Artificial SequenceArtificially Synthesized DNA Sequence
atgaagtgtaaagtttgaaagaaa

SEQ ID NO: 891 - DNA, 24 bp, Artificial Sequence
  Description of Artificial SequenceArtificially Synthesized DNA Sequence
agagagtaagataattgtatagta

SEQ ID NO: 892 - DNA, 24 bp, Artificial Sequence
  Description of Artificial SequenceArtificially Synthesized DNA Sequence
tttatgagatagatgaaataagtg

SEQ ID NO: 893 - DNA, 24 bp, Artificial Sequence
  Description of Artificial SequenceArtificially Synthesized DNA Sequence
agaaattagtagtaatgatttgtg

SEQ ID NO: 894 - DNA, 24 bp, Artificial Sequence
  Description of Artificial SequenceArtificially Synthesized DNA Sequence
gatttgagattgaatgagaatata

SEQ ID NO: 895 - DNA, 24 bp, Artificial Sequence
  Description of Artificial SequenceArtificially Synthesized DNA Sequence
gattagaaagatgaataaagatga

SEQ ID NO: 896 - DNA, 24 bp, Artificial Sequence
  Description of Artificial SequenceArtificially Synthesized DNA Sequence
tagatagaaagtatatgttgtagt

SEQ ID NO: 897 - DNA, 24 bp, Artificial Sequence
  Description of Artificial SequenceArtificially Synthesized DNA Sequence
gaagatagtaaagtaaagtaagtt

SEQ ID NO: 898 - DNA, 24 bp, Artificial Sequence
  Description of Artificial SequenceArtificially Synthesized DNA Sequence
aaatgtgtgtttagtagttgtaaa

SEQ ID NO: 899 - DNA, 24 bp, Artificial Sequence
  Description of Artificial SequenceArtificially Synthesized DNA Sequence
ttgttgaagtaagagatgaataaa

SEQ ID NO: 900 - DNA, 24 bp, Artificial Sequence
  Description of Artificial SequenceArtificially Synthesized DNA Sequence
tatttgagagaaagaaagagttta

SEQ ID NO: 901 - DNA, 24 bp, Artificial Sequence
  Description of Artificial SequenceArtificially Synthesized DNA Sequence
tatttagtgatgaatttgtgatgt

SEQ ID NO: 902 - DNA, 24 bp, Artificial Sequence
  Description of Artificial SequenceArtificially Synthesized DNA Sequence
ttatagtgatgatgataagttgat

SEQ ID NO: 903 - DNA, 24 bp, Artificial Sequence
  Description of Artificial SequenceArtificially Synthesized DNA Sequence
taaagataattgtagaaagtagtg

SEQ ID NO: 904 - DNA, 24 bp, Artificial Sequence
  Description of Artificial SequenceArtificially Synthesized DNA Sequence
gtttagtattgatattgtgtgtaa

SEQ ID NO: 905 - DNA, 24 bp, Artificial Sequence
  Description of Artificial SequenceArtificially Synthesized DNA Sequence
gtgttgtgaataagattgaaatat

SEQ ID NO: 906 - DNA, 24 bp, Artificial Sequence
  Description of Artificial SequenceArtificially Synthesized DNA Sequence
aaagaaagtataaagtgagataga

SEQ ID NO: 907 - DNA, 24 bp, Artificial Sequence
  Description of Artificial SequenceArtificially Synthesized DNA Sequence
tatttgtaagaagtgtagatattg

SEQ ID NO: 908 - DNA, 24 bp, Artificial Sequence
  Description of Artificial SequenceArtificially Synthesized DNA Sequence
tagaagatgaaattgtgatttgtt

SEQ ID NO: 909 - DNA, 24 bp, Artificial Sequence
  Description of Artificial SequenceArtificially Synthesized DNA Sequence
ataatagtaagtgaatgatgagat

SEQ ID NO: 910 - DNA, 24 bp, Artificial Sequence
  Description of Artificial SequenceArtificially Synthesized DNA Sequence
aatgtgaataagataaagtgtgta

SEQ ID NO: 911 - DNA, 24 bp, Artificial Sequence
  Description of Artificial SequenceArtificially Synthesized DNA Sequence
attgaagataaagatgttgtttag

SEQ ID NO: 912 - DNA, 24 bp, Artificial Sequence
  Description of Artificial SequenceArtificially Synthesized DNA Sequence
tgaaatagaagtgagattatagta

SEQ ID NO: 913 - DNA, 24 bp, Artificial Sequence
  Description of Artificial SequenceArtificially Synthesized DNA Sequence
agttattgtgaaagagtttatgat

SEQ ID NO: 914 - DNA, 24 bp, Artificial Sequence
  Description of Artificial SequenceArtificially Synthesized DNA Sequence
aaatagtagtgatagagaagattt

SEQ ID NO: 915 - DNA, 24 bp, Artificial Sequence
  Description of Artificial SequenceArtificially Synthesized DNA Sequence
agtgtatgaagtgtaataagatta

SEQ ID NO: 916 - DNA, 24 bp, Artificial Sequence
  Description of Artificial SequenceArtificially Synthesized DNA Sequence
tgattaagattgtgtagtgttata

SEQ ID NO: 917 - DNA, 24 bp, Artificial Sequence
  Description of Artificial SequenceArtificially Synthesized DNA Sequence
agtttatgatatttgtagatgagt

SEQ ID NO: 918 - DNA, 24 bp, Artificial Sequence
  Description of Artificial SequenceArtificially Synthesized DNA Sequence
tatgtgtatgaagattatagttag

SEQ ID NO: 919 - DNA, 24 bp, Artificial Sequence
  Description of Artificial SequenceArtificially Synthesized DNA Sequence
gaaattgttgtatagagtgatata

SEQ ID NO: 920 - DNA, 24 bp, Artificial Sequence
  Description of Artificial SequenceArtificially Synthesized DNA Sequence
tagaaatagtttaagtatagtgtg

SEQ ID NO: 921 - DNA, 24 bp, Artificial Sequence
  Description of Artificial SequenceArtificially Synthesized DNA Sequence
tgatttagatgtttattgtgagaa

SEQ ID NO: 922 - DNA, 24 bp, Artificial Sequence
  Description of Artificial SequenceArtificially Synthesized DNA Sequence
aagttgatatttgttgttagatga

SEQ ID NO: 923 - DNA, 24 bp, Artificial Sequence
  Description of Artificial SequenceArtificially Synthesized DNA Sequence
tgatgtgataatgagaataaagaa

SEQ ID NO: 924 - DNA, 24 bp, Artificial Sequence
  Description of Artificial SequenceArtificially Synthesized DNA Sequence
aaagtttagtttgtattagtagag

SEQ ID NO: 925 - DNA, 24 bp, Artificial Sequence
  Description of Artificial SequenceArtificially Synthesized DNA Sequence
agtttgatgtgatagtaaatagaa

SEQ ID NO: 926 - DNA, 24 bp, Artificial Sequence
  Description of Artificial SequenceArtificially Synthesized DNA Sequence
aagtgttattgaatgtgatgttat

SEQ ID NO: 927 - DNA, 24 bp, Artificial Sequence
  Description of Artificial SequenceArtificially Synthesized DNA Sequence
aaattgaagtgtgataatgtttgt

SEQ ID NO: 928 - DNA, 24 bp, Artificial Sequence
  Description of Artificial SequenceArtificially Synthesized DNA Sequence
gtttagtgattaaagatagattag

SEQ ID NO: 929 - DNA, 24 bp, Artificial Sequence
  Description of Artificial SequenceArtificially Synthesized DNA Sequence
ataagtgtataagagaagtgttaa

SEQ ID NO: 930 - DNA, 24 bp, Artificial Sequence
  Description of Artificial SequenceArtificially Synthesized DNA Sequence
atgaatttgtttgtgatgaagtta

SEQ ID NO: 931 - DNA, 24 bp, Artificial Sequence
  Description of Artificial SequenceArtificially Synthesized DNA Sequence
aaagaattgagaaatgaaagttag

SEQ ID NO: 932 - DNA, 24 bp, Artificial Sequence
  Description of Artificial SequenceArtificially Synthesized DNA Sequence
agtgtaagagtataaagtatttga

SEQ ID NO: 933 - DNA, 24 bp, Artificial Sequence
  Description of Artificial SequenceArtificially Synthesized DNA Sequence
gaattaagattgttatatgtgagt

SEQ ID NO: 934 - DNA, 24 bp, Artificial Sequence
  Description of Artificial SequenceArtificially Synthesized DNA Sequence
tatgaaagtgttgtttaagtaaga

SEQ ID NO: 935 - DNA, 24 bp, Artificial Sequence
  Description of Artificial SequenceArtificially Synthesized DNA Sequence
taaagtaaatgttatgtgagagaa

SEQ ID NO: 936 - DNA, 24 bp, Artificial Sequence
  Description of Artificial SequenceArtificially Synthesized DNA Sequence
aaagatattgattgagatagagtt

SEQ ID NO: 937 - DNA, 24 bp, Artificial Sequence
  Description of Artificial SequenceArtificially Synthesized DNA Sequence
aagtgatatgaatatgtgagaaat

SEQ ID NO: 938 - DNA, 24 bp, Artificial Sequence
  Description of Artificial SequenceArtificially Synthesized DNA Sequence
aaatagagtttgttaatgtaagtg

SEQ ID NO: 939 - DNA, 24 bp, Artificial Sequence
  Description of Artificial SequenceArtificially Synthesized DNA Sequence
gatttagatgagttaagaatttag

SEQ ID NO: 940 - DNA, 24 bp, Artificial Sequence
  Description of Artificial SequenceArtificially Synthesized DNA Sequence
ttgtaaatgagtgtgaatattgta

SEQ ID NO: 941 - DNA, 24 bp, Artificial Sequence
  Description of Artificial SequenceArtificially Synthesized DNA Sequence
agtagtgtatttgagataatagaa

SEQ ID NO: 942 - DNA, 24 bp, Artificial Sequence
  Description of Artificial SequenceArtificially Synthesized DNA Sequence
tgagttaaagagttgttgatattt

SEQ ID NO: 943 - DNA, 24 bp, Artificial Sequence
  Description of Artificial SequenceArtificially Synthesized DNA Sequence
aaagagtgtattagaaatagtttg

SEQ ID NO: 944 - DNA, 24 bp, Artificial Sequence
  Description of Artificial SequenceArtificially Synthesized DNA Sequence
gtttagttatttgatgagataatg

SEQ ID NO: 945 - DNA, 24 bp, Artificial Sequence
  Description of Artificial SequenceArtificially Synthesized DNA Sequence
aagtgtaaatgaataaagagttgt

SEQ ID NO: 946 - DNA, 24 bp, Artificial Sequence
  Description of Artificial SequenceArtificially Synthesized DNA Sequence
aataaagtgagtagaagtgtaatt

SEQ ID NO: 947 - DNA, 24 bp, Artificial Sequence
  Description of Artificial SequenceArtificially Synthesized DNA Sequence
tattgagtttgtgtaaagaagata

SEQ ID NO: 948 - DNA, 24 bp, Artificial Sequence
  Description of Artificial SequenceArtificially Synthesized DNA Sequence
tttatagttgttgtgttgaaagtt

SEQ ID NO: 949 - DNA, 24 bp, Artificial Sequence
  Description of Artificial SequenceArtificially Synthesized DNA Sequence
atgaaatatgattgtgtttgttgt

SEQ ID NO: 950 - DNA, 24 bp, Artificial Sequence
  Description of Artificial SequenceArtificially Synthesized DNA Sequence
aaagagatgtaaagtgagttatta

SEQ ID NO: 951 - DNA, 24 bp, Artificial Sequence
  Description of Artificial SequenceArtificially Synthesized DNA Sequence
ttgaagaaagttagatgatgaatt

SEQ ID NO: 952 - DNA, 24 bp, Artificial Sequence
  Description of Artificial SequenceArtificially Synthesized DNA Sequence
atgttatttgtttagtttgtgtga

SEQ ID NO: 953 - DNA, 24 bp, Artificial Sequence
  Description of Artificial SequenceArtificially Synthesized DNA Sequence
aaatatgaatttgaagagaagtga

SEQ ID NO: 954 - DNA, 24 bp, Artificial Sequence
  Description of Artificial SequenceArtificially Synthesized DNA Sequence
gattagatatagaatattgaagag

SEQ ID NO: 955 - DNA, 24 bp, Artificial Sequence
  Description of Artificial SequenceArtificially Synthesized DNA Sequence
ttagaataagagaaatgtatgtgt

SEQ ID NO: 956 - DNA, 24 bp, Artificial Sequence
  Description of Artificial SequenceArtificially Synthesized DNA Sequence
tttatgaaagagaagtgtattatg

SEQ ID NO: 957 - DNA, 24 bp, Artificial Sequence
  Description of Artificial SequenceArtificially Synthesized DNA Sequence
gtaagtattaagtgtgatttagta

SEQ ID NO: 958 - DNA, 24 bp, Artificial Sequence
  Description of Artificial SequenceArtificially Synthesized DNA Sequence
ataaagagaagtaaagagtaaagt

SEQ ID NO: 959 - DNA, 24 bp, Artificial Sequence
  Description of Artificial SequenceArtificially Synthesized DNA Sequence
attgttaattgaagtgtatgaaag

SEQ ID NO: 960 - DNA, 24 bp, Artificial Sequence
  Description of Artificial SequenceArtificially Synthesized DNA Sequence
tatatagttgagttgagtaagatt

SEQ ID NO: 961 - DNA, 24 bp, Artificial Sequence
  Description of Artificial SequenceArtificially Synthesized DNA Sequence
tagatgagatatatgaaagatagt

SEQ ID NO: 962 - DNA, 24 bp, Artificial Sequence
  Description of Artificial SequenceArtificially Synthesized DNA Sequence
ataagaagatgatttgtgtaaatg

SEQ ID NO: 963 - DNA, 24 bp, Artificial Sequence
  Description of Artificial SequenceArtificially Synthesized DNA Sequence
ttagtaataagaaagatgaagaga

SEQ ID NO: 964 - DNA, 24 bp, Artificial Sequence
  Description of Artificial SequenceArtificially Synthesized DNA Sequence
gatttgtgagtaaagtaaatagaa

SEQ ID NO: 965 - DNA, 24 bp, Artificial Sequence
  Description of Artificial SequenceArtificially Synthesized DNA Sequence
aaatagatgtagaatttgtgtgtt

SEQ ID NO: 966 - DNA, 24 bp, Artificial Sequence
  Description of Artificial SequenceArtificially Synthesized DNA Sequence
gaaattagtgtttgtgtgtattat

SEQ ID NO: 967 - DNA, 24 bp, Artificial Sequence
  Description of Artificial SequenceArtificially Synthesized DNA Sequence
atttgagtatgatagaagattgtt

SEQ ID NO: 968 - DNA, 24 bp, Artificial Sequence
  Description of Artificial SequenceArtificially Synthesized DNA Sequence
atagagttgaagtatgtaaagttt

SEQ ID NO: 969 - DNA, 24 bp, Artificial Sequence
  Description of Artificial SequenceArtificially Synthesized DNA Sequence
taatttgtgaatgttgttattgtg

SEQ ID NO: 970 - DNA, 24 bp, Artificial Sequence
  Description of Artificial SequenceArtificially Synthesized DNA Sequence
ttagtttatgagagtgagatttaa

SEQ ID NO: 971 - DNA, 24 bp, Artificial Sequence
  Description of Artificial SequenceArtificially Synthesized DNA Sequence
gttgttagagtgtttatgaaattt

SEQ ID NO: 972 - DNA, 24 bp, Artificial Sequence
  Description of Artificial SequenceArtificially Synthesized DNA Sequence
tttattgtgatgtgaaataagaga

SEQ ID NO: 973 - DNA, 24 bp, Artificial Sequence
  Description of Artificial SequenceArtificially Synthesized DNA Sequence
gtaagtaatatgatagtgattaag

SEQ ID NO: 974 - DNA, 24 bp, Artificial Sequence
  Description of Artificial SequenceArtificially Synthesized DNA Sequence
tgagatgatgtatatgtagtaata

SEQ ID NO: 975 - DNA, 24 bp, Artificial Sequence
  Description of Artificial SequenceArtificially Synthesized DNA Sequence
aattgagaaagagataaatgatag

SEQ ID NO: 976 - DNA, 24 bp, Artificial Sequence
  Description of Artificial SequenceArtificially Synthesized DNA Sequence
tttgaagtgatgttagaatgttta

SEQ ID NO: 977 - DNA, 24 bp, Artificial Sequence
  Description of Artificial SequenceArtificially Synthesized DNA Sequence
agttgttgtgtaattgttagtaaa

SEQ ID NO: 978 - DNA, 24 bp, Artificial Sequence
  Description of Artificial SequenceArtificially Synthesized DNA Sequence
atagtgagaagtgataagatattt

SEQ ID NO: 979 - DNA, 24 bp, Artificial Sequence
  Description of Artificial SequenceArtificially Synthesized DNA Sequence
gtgtgataagtaattgagttaaat

SEQ ID NO: 980 - DNA, 24 bp, Artificial Sequence
  Description of Artificial SequenceArtificially Synthesized DNA Sequence
tagttattgtttgtgaatttgaga

SEQ ID NO: 981 - DNA, 24 bp, Artificial Sequence
  Description of Artificial SequenceArtificially Synthesized DNA Sequence
atagttgaatagtaatttgaagag

SEQ ID NO: 982 - DNA, 24 bp, Artificial Sequence
  Description of Artificial SequenceArtificially Synthesized DNA Sequence
atgtttgtgtttgaatagagaata

SEQ ID NO: 983 - DNA, 24 bp, Artificial Sequence
  Description of Artificial SequenceArtificially Synthesized DNA Sequence
tgataaagatatgagagattgtaa

SEQ ID NO: 984 - DNA, 24 bp, Artificial Sequence
  Description of Artificial SequenceArtificially Synthesized DNA Sequence
taaagatgagatgttgttaaagtt

SEQ ID NO: 985 - DNA, 24 bp, Artificial Sequence
  Description of Artificial SequenceArtificially Synthesized DNA Sequence
aagtgaaatttgtaagaattagtg

SEQ ID NO: 986 - DNA, 24 bp, Artificial Sequence
  Description of Artificial SequenceArtificially Synthesized DNA Sequence
gaaatgagagttattgatagttta

SEQ ID NO: 987 - DNA, 24 bp, Artificial Sequence
  Description of Artificial SequenceArtificially Synthesized DNA Sequence
tttgtaaatgagatatagtgttag

SEQ ID NO: 988 - DNA, 24 bp, Artificial Sequence
  Description of Artificial SequenceArtificially Synthesized DNA Sequence
gttaattgtgatatttgattagtg

SEQ ID NO: 989 - DNA, 24 bp, Artificial Sequence
  Description of Artificial SequenceArtificially Synthesized DNA Sequence
agagtgttgataaagatgtttata

SEQ ID NO: 990 - DNA, 24 bp, Artificial Sequence
  Description of Artificial SequenceArtificially Synthesized DNA Sequence
aattgtgagaaattgataagaaga

SEQ ID NO: 991 - DNA, 24 bp, Artificial Sequence
  Description of Artificial SequenceArtificially Synthesized DNA Sequence
ttaaagagaattgagaagagaaat

SEQ ID NO: 992 - DNA, 24 bp, Artificial Sequence
  Description of Artificial SequenceArtificially Synthesized DNA Sequence
ttgttagaagaattgaatgtatgt

SEQ ID NO: 993 - DNA, 24 bp, Artificial Sequence
  Description of Artificial SequenceArtificially Synthesized DNA Sequence
agttaagatatgtgtgatgtttaa

SEQ ID NO: 994 - DNA, 24 bp, Artificial Sequence
  Description of Artificial SequenceArtificially Synthesized DNA Sequence
tgagttatgttgtaatagaaattg

SEQ ID NO: 995 - DNA, 24 bp, Artificial Sequence
  Description of Artificial SequenceArtificially Synthesized DNA Sequence
ttagataagtttagagattgagaa

SEQ ID NO: 996 - DNA, 24 bp, Artificial Sequence
  Description of Artificial SequenceArtificially Synthesized DNA Sequence
atgagtaataagagtatttgaagt

SEQ ID NO: 997 - DNA, 24 bp, Artificial Sequence
  Description of Artificial SequenceArtificially Synthesized DNA Sequence
tgtttaagtgtaatgatttgttag

SEQ ID NO: 998 - DNA, 24 bp, Artificial Sequence
  Description of Artificial SequenceArtificially Synthesized DNA Sequence
ttgaagaagattgttattgttgaa

SEQ ID NO: 999 - DNA, 24 bp, Artificial Sequence
  Description of Artificial SequenceArtificially Synthesized DNA Sequence
tatagaaagattaaagagtgaatg

SEQ ID NO: 1000 - DNA, 24 bp, Artificial Sequence
  Description of Artificial SequenceArtificially Synthesized DNA Sequence
taaattgttagaaatttgagtgtg

SEQ ID NO: 1001 - DNA, 24 bp, Artificial Sequence
  Description of Artificial SequenceArtificially Synthesized DNA Sequence
attgttagtgtgttattgattatg

SEQ ID NO: 1002 - DNA, 24 bp, Artificial Sequence
  Description of Artificial SequenceArtificially Synthesized DNA Sequence
gagaattatgtgtgaatatagaaa

SEQ ID NO: 1003 - DNA, 24 bp, Artificial Sequence
  Description of Artificial SequenceArtificially Synthesized DNA Sequence
ttgattgataaagtaaagagtgta

SEQ ID NO: 1004 - DNA, 24 bp, Artificial Sequence
  Description of Artificial SequenceArtificially Synthesized DNA Sequence
gtgtgtaaattgaatatgttaatg

SEQ ID NO: 1005 - DNA, 24 bp, Artificial Sequence
  Description of Artificial SequenceArtificially Synthesized DNA Sequence
aaagtaaagaaagaagtttgaaag

SEQ ID NO: 1006 - DNA, 24 bp, Artificial Sequence
  Description of Artificial SequenceArtificially Synthesized DNA Sequence
tttagttgaagaatagaaagaaag

SEQ ID NO: 1007 - DNA, 24 bp, Artificial Sequence
  Description of Artificial SequenceArtificially Synthesized DNA Sequence
gtgtaataagagtgaatagtaatt

SEQ ID NO: 1008 - DNA, 24 bp, Artificial Sequence
  Description of Artificial SequenceArtificially Synthesized DNA Sequence
tattgaaataagagagatttgtga

SEQ ID NO: 1009 - DNA, 24 bp, Artificial Sequence
  Description of Artificial SequenceArtificially Synthesized DNA Sequence
atgagaaagaagaagttaagattt

SEQ ID NO: 1010 - DNA, 24 bp, Artificial Sequence
  Description of Artificial SequenceArtificially Synthesized DNA Sequence
aagagtgagtatattgttaaagaa

SEQ ID NO: 1011 - DNA, 24 bp, Artificial Sequence
  Description of Artificial SequenceArtificially Synthesized DNA Sequence
tttgtaaagtgatgatgtaagata

SEQ ID NO: 1012 - DNA, 24 bp, Artificial Sequence
  Description of Artificial SequenceArtificially Synthesized DNA Sequence
gatgttatgtgatgaaatatgtat

SEQ ID NO: 1013 - DNA, 24 bp, Artificial Sequence
  Description of Artificial SequenceArtificially Synthesized DNA Sequence
gtagaataaagtgttaaagtgtta

SEQ ID NO: 1014 - DNA, 24 bp, Artificial Sequence
  Description of Artificial SequenceArtificially Synthesized DNA Sequence
aaagagtatgtgtgtatgatattt

SEQ ID NO: 1015 - DNA, 24 bp, Artificial Sequence
  Description of Artificial SequenceArtificially Synthesized DNA Sequence
aaagataagagttagtaaattgtg

SEQ ID NO: 1016 - DNA, 24 bp, Artificial Sequence
  Description of Artificial SequenceArtificially Synthesized DNA Sequence
aagaattagagaataagtgtgata

SEQ ID NO: 1017 - DNA, 24 bp, Artificial Sequence
  Description of Artificial SequenceArtificially Synthesized DNA Sequence
gataagaaagtgaaatgtaaattg

SEQ ID NO: 1018 - DNA, 24 bp, Artificial Sequence
  Description of Artificial SequenceArtificially Synthesized DNA Sequence
gatgaaagatgtttaaagtttgtt

SEQ ID NO: 1019 - DNA, 24 bp, Artificial Sequence
  Description of Artificial SequenceArtificially Synthesized DNA Sequence
agtgtaagtaataagtttgagaaa

SEQ ID NO: 1020 - DNA, 24 bp, Artificial Sequence
  Description of Artificial SequenceArtificially Synthesized DNA Sequence
gttgagaattagaatttgataaag

SEQ ID NO: 1021 - DNA, 24 bp, Artificial Sequence
  Description of Artificial SequenceArtificially Synthesized DNA Sequence
ttaagaaatttgtatgtgttgttg

SEQ ID NO: 1022 - DNA, 24 bp, Artificial Sequence
  Description of Artificial SequenceArtificially Synthesized DNA Sequence
agaagatttagatgaaatgagttt

SEQ ID NO: 1023 - DNA, 24 bp, Artificial Sequence
  Description of Artificial SequenceArtificially Synthesized DNA Sequence
taagtttgagataaagatgatatg

SEQ ID NO: 1024 - DNA, 24 bp, Artificial Sequence
  Description of Artificial SequenceArtificially Synthesized DNA Sequence
tgagatagtttgtaatatgtttgt

SEQ ID NO: 1025 - DNA, 24 bp, Artificial Sequence
  Description of Artificial SequenceArtificially Synthesized DNA Sequence
agtttgaaattgtaagtttgatga

SEQ ID NO: 1026 - DNA, 24 bp, Artificial Sequence
  Description of Artificial SequenceArtificially Synthesized DNA Sequence
tagaattgattaatgatgagtagt

SEQ ID NO: 1027 - DNA, 24 bp, Artificial Sequence
  Description of Artificial SequenceArtificially Synthesized DNA Sequence
agagatttgtaataagtattgaag

SEQ ID NO: 1028 - DNA, 24 bp, Artificial Sequence
  Description of Artificial SequenceArtificially Synthesized DNA Sequence
ataatgatgtaatgtaagtagtgt

SEQ ID NO: 1029 - DNA, 24 bp, Artificial Sequence
  Description of Artificial SequenceArtificially Synthesized DNA Sequence
tgaaatttgatgagagatatgtta

SEQ ID NO: 1030 - DNA, 24 bp, Artificial Sequence
  Description of Artificial SequenceArtificially Synthesized DNA Sequence
tgtgtaaagtatagtttatgttag

SEQ ID NO: 1031 - DNA, 24 bp, Artificial Sequence
  Description of Artificial SequenceArtificially Synthesized DNA Sequence
tgaataagtgaaatagaatgaatg

SEQ ID NO: 1032 - DNA, 24 bp, Artificial Sequence
  Description of Artificial SequenceArtificially Synthesized DNA Sequence
aaagaaagattgtaataagtagag

SEQ ID NO: 1033 - DNA, 24 bp, Artificial Sequence
  Description of Artificial SequenceArtificially Synthesized DNA Sequence
aatgaaatagtgttaaatgagtgt

SEQ ID NO: 1034 - DNA, 24 bp, Artificial Sequence
  Description of Artificial SequenceArtificially Synthesized DNA Sequence
gtagataaagatgtgaattatgat

SEQ ID NO: 1035 - DNA, 24 bp, Artificial Sequence
  Description of Artificial SequenceArtificially Synthesized DNA Sequence
gatagtatatgtgtgtatttgttt

SEQ ID NO: 1036 - DNA, 24 bp, Artificial Sequence
  Description of Artificial SequenceArtificially Synthesized DNA Sequence
atgtttgtagaaatgtttgaagat

SEQ ID NO: 1037 - DNA, 24 bp, Artificial Sequence
  Description of Artificial SequenceArtificially Synthesized DNA Sequence
aaatttgtagagagaaatttgttg

SEQ ID NO: 1038 - DNA, 24 bp, Artificial Sequence
  Description of Artificial SequenceArtificially Synthesized DNA Sequence
tagaataagattagtaagtgtaga

SEQ ID NO: 1039 - DNA, 24 bp, Artificial Sequence
  Description of Artificial SequenceArtificially Synthesized DNA Sequence
tgatttagagaaatatgagtagaa

SEQ ID NO: 1040 - DNA, 24 bp, Artificial Sequence
  Description of Artificial SequenceArtificially Synthesized DNA Sequence
aatagagtatgttgtttatgagaa

SEQ ID NO: 1041 - DNA, 24 bp, Artificial Sequence
  Description of Artificial SequenceArtificially Synthesized DNA Sequence
gatgatgaagagtttattgtaaat

SEQ ID NO: 1042 - DNA, 24 bp, Artificial Sequence
  Description of Artificial SequenceArtificially Synthesized DNA Sequence
aagtaaagaagaagaaatgtgtta

SEQ ID NO: 1043 - DNA, 24 bp, Artificial Sequence
  Description of Artificial SequenceArtificially Synthesized DNA Sequence
ttgaagaattaagtgtttagtgta

SEQ ID NO: 1044 - DNA, 24 bp, Artificial Sequence
  Description of Artificial SequenceArtificially Synthesized DNA Sequence
agaaagaatgttgatttatgatgt

SEQ ID NO: 1045 - DNA, 24 bp, Artificial Sequence
  Description of Artificial SequenceArtificially Synthesized DNA Sequence
gattaaagagatgttgattgaaat

SEQ ID NO: 1046 - DNA, 24 bp, Artificial Sequence
  Description of Artificial SequenceArtificially Synthesized DNA Sequence
aatgataattgttgagagagtaat

SEQ ID NO: 1047 - DNA, 24 bp, Artificial Sequence
  Description of Artificial SequenceArtificially Synthesized DNA Sequence
gtttgttgaaagtgtaaagtatat

SEQ ID NO: 1048 - DNA, 24 bp, Artificial Sequence
  Description of Artificial SequenceArtificially Synthesized DNA Sequence
tgagttatatgagaaagtgtaatt

SEQ ID NO: 1049 - DNA, 24 bp, Artificial Sequence
  Description of Artificial SequenceArtificially Synthesized DNA Sequence
ttgtgagaaagaagtatatagaat

SEQ ID NO: 1050 - DNA, 24 bp, Artificial Sequence
  Description of Artificial SequenceArtificially Synthesized DNA Sequence
gtaagtttagagttatagagttta

SEQ ID NO: 1051 - DNA, 24 bp, Artificial Sequence
  Description of Artificial SequenceArtificially Synthesized DNA Sequence
gatagatagataagttaattgaag

SEQ ID NO: 1052 - DNA, 24 bp, Artificial Sequence
  Description of Artificial SequenceArtificially Synthesized DNA Sequence
agagatgattgtttatgtattatg

SEQ ID NO: 1053 - DNA, 24 bp, Artificial Sequence
  Description of Artificial SequenceArtificially Synthesized DNA Sequence
aaagttaagaaattgtagtgatag

SEQ ID NO: 1054 - DNA, 24 bp, Artificial Sequence
  Description of Artificial SequenceArtificially Synthesized DNA Sequence
tttgatattgtttgtgagtgtata

SEQ ID NO: 1055 - DNA, 24 bp, Artificial Sequence
  Description of Artificial SequenceArtificially Synthesized DNA Sequence
atttgtagaaagttgttatgagtt

SEQ ID NO: 1056 - DNA, 24 bp, Artificial Sequence
  Description of Artificial SequenceArtificially Synthesized DNA Sequence
gatttgagtaagtttatagatgaa

SEQ ID NO: 1057 - DNA, 24 bp, Artificial Sequence
  Description of Artificial SequenceArtificially Synthesized DNA Sequence
aagataaagtgagttgatttagat

SEQ ID NO: 1058 - DNA, 24 bp, Artificial Sequence
  Description of Artificial SequenceArtificially Synthesized DNA Sequence
gatattgtaagatatgttgtaaag

SEQ ID NO: 1059 - DNA, 24 bp, Artificial Sequence
  Description of Artificial SequenceArtificially Synthesized DNA Sequence
gtaagagtgtattgtaagttaatt

SEQ ID NO: 1060 - DNA, 24 bp, Artificial Sequence
  Description of Artificial SequenceArtificially Synthesized DNA Sequence
gtgtgattagtaatgaagtattta

SEQ ID NO: 1061 - DNA, 24 bp, Artificial Sequence
  Description of Artificial SequenceArtificially Synthesized DNA Sequence
gtaagaaagattaagtgttagtaa

SEQ ID NO: 1062 - DNA, 24 bp, Artificial Sequence
  Description of Artificial SequenceArtificially Synthesized DNA Sequence
agtagaaagttgaaattgattatg

SEQ ID NO: 1063 - DNA, 24 bp, Artificial Sequence
  Description of Artificial SequenceArtificially Synthesized DNA Sequence
taagagaagttgagtaatgtattt

SEQ ID NO: 1064 - DNA, 24 bp, Artificial Sequence
  Description of Artificial SequenceArtificially Synthesized DNA Sequence
gttaagaaatagtagataagtgaa

SEQ ID NO: 1065 - DNA, 24 bp, Artificial Sequence
  Description of Artificial SequenceArtificially Synthesized DNA Sequence
taagtaaattgaaagtgtatagtg

SEQ ID NO: 1066 - DNA, 24 bp, Artificial Sequence
  Description of Artificial SequenceArtificially Synthesized DNA Sequence
aagatgtatgtttattgttgtgta

SEQ ID NO: 1067 - DNA, 24 bp, Artificial Sequence
  Description of Artificial SequenceArtificially Synthesized DNA Sequence
atttagaatatagtgaagagatag

SEQ ID NO: 1068 - DNA, 24 bp, Artificial Sequence
  Description of Artificial SequenceArtificially Synthesized DNA Sequence
gttatgaaagagtatgtgttaaat

SEQ ID NO: 1069 - DNA, 24 bp, Artificial Sequence
  Description of Artificial SequenceArtificially Synthesized DNA Sequence
tattatgtgaagaagaatgattag

SEQ ID NO: 1070 - DNA, 24 bp, Artificial Sequence
  Description of Artificial SequenceArtificially Synthesized DNA Sequence
taataagttgaagagaattgttgt

SEQ ID NO: 1071 - DNA, 24 bp, Artificial Sequence
  Description of Artificial SequenceArtificially Synthesized DNA Sequence
tgatgtttgatgtaattgttaaag

SEQ ID NO: 1072 - DNA, 24 bp, Artificial Sequence
  Description of Artificial SequenceArtificially Synthesized DNA Sequence
gtgaaagatttgagtttgtataat

SEQ ID NO: 1073 - DNA, 24 bp, Artificial Sequence
  Description of Artificial SequenceArtificially Synthesized DNA Sequence
agagaatatagattgagatttgtt

SEQ ID NO: 1074 - DNA, 24 bp, Artificial Sequence
  Description of Artificial SequenceArtificially Synthesized DNA Sequence
tttgagatgtgatgataaagttaa

SEQ ID NO: 1075 - DNA, 24 bp, Artificial Sequence
  Description of Artificial SequenceArtificially Synthesized DNA Sequence
gttgtaaattgtagtaaagaagta

SEQ ID NO: 1076 - DNA, 24 bp, Artificial Sequence
  Description of Artificial SequenceArtificially Synthesized DNA Sequence
gtgttatgatgttgtttgtattat

SEQ ID NO: 1077 - DNA, 24 bp, Artificial Sequence
  Description of Artificial SequenceArtificially Synthesized DNA Sequence
attattgtgtagatgtattaagag

SEQ ID NO: 1078 - DNA, 24 bp, Artificial Sequence
  Description of Artificial SequenceArtificially Synthesized DNA Sequence
gttagaaagatttagaagttagtt

SEQ ID NO: 1079 - DNA, 24 bp, Artificial Sequence
  Description of Artificial SequenceArtificially Synthesized DNA Sequence
ttgtgtattaagagagtgaaatat

SEQ ID NO: 1080 - DNA, 24 bp, Artificial Sequence
  Description of Artificial SequenceArtificially Synthesized DNA Sequence
gtttaagatagaaagagtgattta

SEQ ID NO: 1081 - DNA, 24 bp, Artificial Sequence
  Description of Artificial SequenceArtificially Synthesized DNA Sequence
aatgagaaatagatagttattgtg

SEQ ID NO: 1082 - DNA, 24 bp, Artificial Sequence
  Description of Artificial SequenceArtificially Synthesized DNA Sequence
tgaattgaataagaatttgttgtg

SEQ ID NO: 1083 - DNA, 24 bp, Artificial Sequence
  Description of Artificial SequenceArtificially Synthesized DNA Sequence
aataagattgaattagtgagtaag

SEQ ID NO: 1084 - DNA, 24 bp, Artificial Sequence
  Description of Artificial SequenceArtificially Synthesized DNA Sequence
aatgtttgagagatttagtaaaga

SEQ ID NO: 1085 - DNA, 24 bp, Artificial Sequence
  Description of Artificial SequenceArtificially Synthesized DNA Sequence
agtttagaatagaaatgtgtttga

SEQ ID NO: 1086 - DNA, 24 bp, Artificial Sequence
  Description of Artificial SequenceArtificially Synthesized DNA Sequence
tataagtaagtgttaagatttgag

SEQ ID NO: 1087 - DNA, 24 bp, Artificial Sequence
  Description of Artificial SequenceArtificially Synthesized DNA Sequence
gtagtgaataagttagtgttaata

SEQ ID NO: 1088 - DNA, 24 bp, Artificial Sequence
  Description of Artificial SequenceArtificially Synthesized DNA Sequence
aagtgtgttaaagtaaatgtagat

SEQ ID NO: 1089 - DNA, 24 bp, Artificial Sequence
  Description of Artificial SequenceArtificially Synthesized DNA Sequence
agagatgtttatgttgtgaattaa

SEQ ID NO: 1090 - DNA, 24 bp, Artificial Sequence
  Description of Artificial SequenceArtificially Synthesized DNA Sequence
agttgaatattgatgataagaaga

SEQ ID NO: 1091 - DNA, 24 bp, Artificial Sequence
  Description of Artificial SequenceArtificially Synthesized DNA Sequence
tgaatgtgagatgtttagaataat

SEQ ID NO: 1092 - DNA, 24 bp, Artificial Sequence
  Description of Artificial SequenceArtificially Synthesized DNA Sequence
aataatgatgtaagtttgagtttg

SEQ ID NO: 1093 - DNA, 24 bp, Artificial Sequence
  Description of Artificial SequenceArtificially Synthesized DNA Sequence
aaagagtgaatagaaataagagaa

SEQ ID NO: 1094 - DNA, 24 bp, Artificial Sequence
  Description of Artificial SequenceArtificially Synthesized DNA Sequence
aataaagttattgagagagtttag

SEQ ID NO: 1095 - DNA, 24 bp, Artificial Sequence
  Description of Artificial SequenceArtificially Synthesized DNA Sequence
agtagtgttgtagtttagtatata

SEQ ID NO: 1096 - DNA, 24 bp, Artificial Sequence
  Description of Artificial SequenceArtificially Synthesized DNA Sequence
gtaagaatgtattagatatttgtg

SEQ ID NO: 1097 - DNA, 24 bp, Artificial Sequence
  Description of Artificial SequenceArtificially Synthesized DNA Sequence
gataaatgtttgataaagtagttg

SEQ ID NO: 1098 - DNA, 24 bp, Artificial Sequence
  Description of Artificial SequenceArtificially Synthesized DNA Sequence
atagtatgtatgtgtgaagattta

SEQ ID NO: 1099 - DNA, 24 bp, Artificial Sequence
  Description of Artificial SequenceArtificially Synthesized DNA Sequence
atgaatgtagagtgattagtttaa

SEQ ID NO: 1100 - DNA, 24 bp, Artificial Sequence
  Description of Artificial SequenceArtificially Synthesized DNA Sequence
gtagtatttagtgatgtaagaata

SEQ ID NO: 1101 - DNA, 24 bp, Artificial Sequence
  Description of Artificial SequenceArtificially Synthesized DNA Sequence
agaattgtattgaagaagaatatg

SEQ ID NO: 1102 - DNA, 24 bp, Artificial Sequence
  Description of Artificial SequenceArtificially Synthesized DNA Sequence
tttatagaattgagagaagttaag

SEQ ID NO: 1103 - DNA, 24 bp, Artificial Sequence
  Description of Artificial SequenceArtificially Synthesized DNA Sequence
aaagtagtagagatttgagaatta

SEQ ID NO: 1104 - DNA, 24 bp, Artificial Sequence
  Description of Artificial SequenceArtificially Synthesized DNA Sequence
tttaaagaaagtattgtaagagtg

SEQ ID NO: 1105 - DNA, 24 bp, Artificial Sequence
  Description of Artificial SequenceArtificially Synthesized DNA Sequence
aaattgagaaagtgaatgaagttt

SEQ ID NO: 1106 - DNA, 24 bp, Artificial Sequence
  Description of Artificial SequenceArtificially Synthesized DNA Sequence
aagaaataagtatgatagtagtag

SEQ ID NO: 1107 - DNA, 24 bp, Artificial Sequence
  Description of Artificial SequenceArtificially Synthesized DNA Sequence
atttgaattgtattgtagtttgtg

SEQ ID NO: 1108 - DNA, 24 bp, Artificial Sequence
  Description of Artificial SequenceArtificially Synthesized DNA Sequence
aagagaataatgtagagatataag

SEQ ID NO: 1109 - DNA, 24 bp, Artificial Sequence
  Description of Artificial SequenceArtificially Synthesized DNA Sequence
tgtgtaatagttgttaatgagtaa

SEQ ID NO: 1110 - DNA, 24 bp, Artificial Sequence
  Description of Artificial SequenceArtificially Synthesized DNA Sequence
tatagttgtagtttagatgaatgt

SEQ ID NO: 1111 - DNA, 24 bp, Artificial Sequence
  Description of Artificial SequenceArtificially Synthesized DNA Sequence
attgtgttagaatgatgttaatag

SEQ ID NO: 1112 - DNA, 24 bp, Artificial Sequence
  Description of Artificial SequenceArtificially Synthesized DNA Sequence
gtttgtatagtatttgattgatgt

SEQ ID NO: 1113 - DNA, 24 bp, Artificial Sequence
  Description of Artificial SequenceArtificially Synthesized DNA Sequence
agagtaaagtatgagttatgaata

SEQ ID NO: 1114 - DNA, 24 bp, Artificial Sequence
  Description of Artificial SequenceArtificially Synthesized DNA Sequence
gaaagtttaagtgatgtatattgt

SEQ ID NO: 1115 - DNA, 24 bp, Artificial Sequence
  Description of Artificial SequenceArtificially Synthesized DNA Sequence
ttaaatgataaagagtagtgaagt

SEQ ID NO: 1116 - DNA, 24 bp, Artificial Sequence
  Description of Artificial SequenceArtificially Synthesized DNA Sequence
ttaaatgtgtgagaagatgaataa

SEQ ID NO: 1117 - DNA, 24 bp, Artificial Sequence
  Description of Artificial SequenceArtificially Synthesized DNA Sequence
atttgtataaagtgaagaagagaa

SEQ ID NO: 1118 - DNA, 24 bp, Artificial Sequence
  Description of Artificial SequenceArtificially Synthesized DNA Sequence
tgattagtatttgtgaagagattt

SEQ ID NO: 1119 - DNA, 24 bp, Artificial Sequence
  Description of Artificial SequenceArtificially Synthesized DNA Sequence
tttgaatgaaattgatgatagatg

SEQ ID NO: 1120 - DNA, 24 bp, Artificial Sequence
  Description of Artificial SequenceArtificially Synthesized DNA Sequence
agagtaagattaagaataagaaag

SEQ ID NO: 1121 - DNA, 24 bp, Artificial Sequence
  Description of Artificial SequenceArtificially Synthesized DNA Sequence
attgaattgagaagtgaagtaaat

SEQ ID NO: 1122 - DNA, 24 bp, Artificial Sequence
  Description of Artificial SequenceArtificially Synthesized DNA Sequence
tttagagaagtattgtttgaaaga

SEQ ID NO: 1123 - DNA, 24 bp, Artificial Sequence
  Description of Artificial SequenceArtificially Synthesized DNA Sequence
taaagtgaaagatttgaaatgatg

SEQ ID NO: 1124 - DNA, 24 bp, Artificial Sequence
  Description of Artificial SequenceArtificially Synthesized DNA Sequence
gaaagttagagaaatgtagaaatt

SEQ ID NO: 1125 - DNA, 24 bp, Artificial Sequence
  Description of Artificial SequenceArtificially Synthesized DNA Sequence
gtgaataatgaagaagttatgtta

SEQ ID NO: 1126 - DNA, 24 bp, Artificial Sequence
  Description of Artificial SequenceArtificially Synthesized DNA Sequence
ttgtgaataaagtagatgtgttat

SEQ ID NO: 1127 - DNA, 24 bp, Artificial Sequence
  Description of Artificial SequenceArtificially Synthesized DNA Sequence
ttatatgatatgagtttgtgttga

SEQ ID NO: 1128 - DNA, 24 bp, Artificial Sequence
  Description of Artificial SequenceArtificially Synthesized DNA Sequence
ttgatttgtgtgagtattagttat

SEQ ID NO: 1129 - DNA, 24 bp, Artificial Sequence
  Description of Artificial SequenceArtificially Synthesized DNA Sequence
aaagtgattaagttagtttgagat

SEQ ID NO: 1130 - DNA, 24 bp, Artificial Sequence
  Description of Artificial SequenceArtificially Synthesized DNA Sequence
ttgtatttgtataatgttgaagag

SEQ ID NO: 1131 - DNA, 24 bp, Artificial Sequence
  Description of Artificial SequenceArtificially Synthesized DNA Sequence
gtttgaaattagtgtgagaaatat

SEQ ID NO: 1132 - DNA, 24 bp, Artificial Sequence
  Description of Artificial SequenceArtificially Synthesized DNA Sequence
aatgttgagattgataatgttgaa

SEQ ID NO: 1133 - DNA, 24 bp, Artificial Sequence
  Description of Artificial SequenceArtificially Synthesized DNA Sequence
tagtagtagtattgttgtaataag

SEQ ID NO: 1134 - DNA, 24 bp, Artificial Sequence
  Description of Artificial SequenceArtificially Synthesized DNA Sequence
gttgtaatttgagtgttagttatt

SEQ ID NO: 1135 - DNA, 24 bp, Artificial Sequence
  Description of Artificial SequenceArtificially Synthesized DNA Sequence
tgaatatgatagttagtaattgtg

SEQ ID NO: 1136 - DNA, 24 bp, Artificial Sequence
  Description of Artificial SequenceArtificially Synthesized DNA Sequence
tgatagtatgtttgtgattaaaga

SEQ ID NO: 1137 - DNA, 24 bp, Artificial Sequence
  Description of Artificial SequenceArtificially Synthesized DNA Sequence
gatgtataaagagtatgttataag

SEQ ID NO: 1138 - DNA, 24 bp, Artificial Sequence
  Description of Artificial SequenceArtificially Synthesized DNA Sequence
agtgagatttagaagatgttatta

SEQ ID NO: 1139 - DNA, 24 bp, Artificial Sequence
  Description of Artificial SequenceArtificially Synthesized DNA Sequence
atgagaatttgttaaagagaaagt

SEQ ID NO: 1140 - DNA, 24 bp, Artificial Sequence
  Description of Artificial SequenceArtificially Synthesized DNA Sequence
aaagaattagtatgatagatgaga

SEQ ID NO: 1141 - DNA, 24 bp, Artificial Sequence
  Description of Artificial SequenceArtificially Synthesized DNA Sequence
tagagttgtatagtttatagttga

SEQ ID NO: 1142 - DNA, 24 bp, Artificial Sequence
  Description of Artificial SequenceArtificially Synthesized DNA Sequence
gtagaatgattgtttagaagattt

SEQ ID NO: 1143 - DNA, 24 bp, Artificial Sequence
  Description of Artificial SequenceArtificially Synthesized DNA Sequence
gtttatgtttgagaagagttattt

SEQ ID NO: 1144 - DNA, 24 bp, Artificial Sequence
  Description of Artificial SequenceArtificially Synthesized DNA Sequence
tagaagtttgaaagttattgattg

SEQ ID NO: 1145 - DNA, 24 bp, Artificial Sequence
  Description of Artificial SequenceArtificially Synthesized DNA Sequence
gatgaagagtatttgttatatgta

SEQ ID NO: 1146 - DNA, 24 bp, Artificial Sequence
  Description of Artificial SequenceArtificially Synthesized DNA Sequence
gatgaatatagtaagtattgagta

SEQ ID NO: 1147 - DNA, 24 bp, Artificial Sequence
  Description of Artificial SequenceArtificially Synthesized DNA Sequence
tagtgatgaaatttgagatagata

SEQ ID NO: 1148 - DNA, 24 bp, Artificial Sequence
  Description of Artificial SequenceArtificially Synthesized DNA Sequence
gaaagaaattgaagagtttgatat

SEQ ID NO: 1149 - DNA, 24 bp, Artificial Sequence
  Description of Artificial SequenceArtificially Synthesized DNA Sequence
atttgagtatttgtgtattgaatg

SEQ ID NO: 1150 - DNA, 24 bp, Artificial Sequence
  Description of Artificial SequenceArtificially Synthesized DNA Sequence
atgagttgaaatttgaagtattgt

SEQ ID NO: 1151 - DNA, 24 bp, Artificial Sequence
  Description of Artificial SequenceArtificially Synthesized DNA Sequence
ttaatagtgagagagtatatgtaa

SEQ ID NO: 1152 - DNA, 24 bp, Artificial Sequence
  Description of Artificial SequenceArtificially Synthesized DNA Sequence
attaagagagtgagtaaatgtaaa

SEQ ID NO: 1153 - DNA, 24 bp, Artificial Sequence
  Description of Artificial SequenceArtificially Synthesized DNA Sequence
aagaatagatgagattagaaatag

SEQ ID NO: 1154 - DNA, 24 bp, Artificial Sequence
  Description of Artificial SequenceArtificially Synthesized DNA Sequence
agtttaaagagttagaattgaaag

SEQ ID NO: 1155 - DNA, 24 bp, Artificial Sequence
  Description of Artificial SequenceArtificially Synthesized DNA Sequence
gtaagatttgttgaataaagaaga

SEQ ID NO: 1156 - DNA, 24 bp, Artificial Sequence
  Description of Artificial SequenceArtificially Synthesized DNA Sequence
agagaaagaagttaaagtgatatt

SEQ ID NO: 1157 - DNA, 24 bp, Artificial Sequence
  Description of Artificial SequenceArtificially Synthesized DNA Sequence
taatagagaagagatgtatgaata

SEQ ID NO: 1158 - DNA, 24 bp, Artificial Sequence
  Description of Artificial SequenceArtificially Synthesized DNA Sequence
ttattagtgataagtgaagtttag

SEQ ID NO: 1159 - DNA, 24 bp, Artificial Sequence
  Description of Artificial SequenceArtificially Synthesized DNA Sequence
ataatgtaaagatgagtttatgag

SEQ ID NO: 1160 - DNA, 24 bp, Artificial Sequence
  Description of Artificial SequenceArtificially Synthesized DNA Sequence
ttgatttgagagttgataagattt

SEQ ID NO: 1161 - DNA, 24 bp, Artificial Sequence
  Description of Artificial SequenceArtificially Synthesized DNA Sequence
atgattattgtgtgtagaattaga

SEQ ID NO: 1162 - DNA, 24 bp, Artificial Sequence
  Description of Artificial SequenceArtificially Synthesized DNA Sequence
tataaagatatagtagatgatgtg

SEQ ID NO: 1163 - DNA, 24 bp, Artificial Sequence
  Description of Artificial SequenceArtificially Synthesized DNA Sequence
tttagttgagatgaagttattaga

SEQ ID NO: 1164 - DNA, 24 bp, Artificial Sequence
  Description of Artificial SequenceArtificially Synthesized DNA Sequence
attgaattgatatagtgtaaagtg

SEQ ID NO: 1165 - DNA, 24 bp, Artificial Sequence
  Description of Artificial SequenceArtificially Synthesized DNA Sequence
gaagaaagattattgtattgagtt

SEQ ID NO: 1166 - DNA, 24 bp, Artificial Sequence
  Description of Artificial SequenceArtificially Synthesized DNA Sequence
attgagtgtagtgatttagaaata

SEQ ID NO: 1167 - DNA, 24 bp, Artificial Sequence
  Description of Artificial SequenceArtificially Synthesized DNA Sequence
aataaagtgtttaagagtagagta

SEQ ID NO: 1168 - DNA, 24 bp, Artificial Sequence
  Description of Artificial SequenceArtificially Synthesized DNA Sequence
gtagagataattgatgtgtaattt

SEQ ID NO: 1169 - DNA, 19 bp, Artificial Sequence
  Description of Artificial SequenceArtificially Synthesized DNA Sequence
tgatcgtagctacgccgcg

SEQ ID NO: 1170 - DNA, 16 bp, Artificial Sequence
  Description of Artificial SequenceArtificially Synthesized DNA Sequence
cgtacgattgcaacgt

SEQ ID NO: 1171 - DNA, 48 bp, Artificial Sequence
  Description of Artificial SequenceArtificially Synthesized DNA Sequence
gattgtaagatttgataaagtgtatccagggaagcgtgtcaccgtcgt

SEQ ID NO: 1172 - DNA, 24 bp, Artificial Sequence
  Description of Artificial SequenceArtificially Synthesized DNA Sequence
tcatggcgactgtccagctttgtg

SEQ ID NO: 1173 - DNA, 24 bp, Artificial Sequence
  Description of Artificial SequenceArtificially Synthesized DNA Sequence
gatttgtattgattgagattaaag

SEQ ID NO: 1174 - DNA, 24 bp, Artificial Sequence
  Description of Artificial SequenceArtificially Synthesized DNA Sequence
tgattgtagtatgtattgataaag

SEQ ID NO: 1175 - DNA, 24 bp, Artificial Sequence
  Description of Artificial SequenceArtificially Synthesized DNA Sequence
gattgtaagatttgataaagtgta

SEQ ID NO: 1176 - DNA, 24 bp, Artificial Sequence
  Description of Artificial SequenceArtificially Synthesized DNA Sequence
gatttgaagattattggtaatgta

SEQ ID NO: 1177 - DNA, 24 bp, Artificial Sequence
  Description of Artificial SequenceArtificially Synthesized DNA Sequence
gattgattattgtgatttgaattg

SEQ ID NO: 1178 - DNA, 24 bp, Artificial Sequence
  Description of Artificial SequenceArtificially Synthesized DNA Sequence
gatttgattgtaaaagattgttga

SEQ ID NO: 1179 - DNA, 24 bp, Artificial Sequence
  Description of Artificial SequenceArtificially Synthesized DNA Sequence
attggtaaattggtaaatgaattg

SEQ ID NO: 1180 - DNA, 24 bp, Artificial Sequence
  Description of Artificial SequenceArtificially Synthesized DNA Sequence
attggatttgataaaggtaaatga

SEQ ID NO: 1181 - DNA, 24 bp, Artificial Sequence
  Description of Artificial SequenceArtificially Synthesized DNA Sequence
gtaagtaatgaatgtaaaaggatt

SEQ ID NO: 1182 - DNA, 24 bp, Artificial Sequence
  Description of Artificial SequenceArtificially Synthesized DNA Sequence
gattgattgattgattgatttgat

SEQ ID NO: 1183 - DNA, 24 bp, Artificial Sequence
  Description of Artificial SequenceArtificially Synthesized DNA Sequence
tgatgattaaagaaagtgattgat

SEQ ID NO: 1184 - DNA, 24 bp, Artificial Sequence
  Description of Artificial SequenceArtificially Synthesized DNA Sequence
aaaggatttgattgataaagtgat

SEQ ID NO: 1185 - DNA, 24 bp, Artificial Sequence
  Description of Artificial SequenceArtificially Synthesized DNA Sequence
tgtagatttgtatgtatgtatgat

SEQ ID NO: 1186 - DNA, 24 bp, Artificial Sequence
  Description of Artificial SequenceArtificially Synthesized DNA Sequence
gatttgataaagaaaggattgatt

SEQ ID NO: 1187 - DNA, 24 bp, Artificial Sequence
  Description of Artificial SequenceArtificially Synthesized DNA Sequence
gattaaagtgattgatgatttgta

SEQ ID NO: 1188 - DNA, 24 bp, Artificial Sequence
  Description of Artificial SequenceArtificially Synthesized DNA Sequence
aaagaaagaaagaaagaaagtgta

SEQ ID NO: 1189 - DNA, 24 bp, Artificial Sequence
  Description of Artificial SequenceArtificially Synthesized DNA Sequence
tgtaaaaggattgatttgtatgta

SEQ ID NO: 1190 - DNA, 24 bp, Artificial Sequence
  Description of Artificial SequenceArtificially Synthesized DNA Sequence
aaagtgtagattgattaaagaaag

SEQ ID NO: 1191 - DNA, 24 bp, Artificial Sequence
  Description of Artificial SequenceArtificially Synthesized DNA Sequence
aaagttgattgattgaaaaggtat

SEQ ID NO: 1192 - DNA, 24 bp, Artificial Sequence
  Description of Artificial SequenceArtificially Synthesized DNA Sequence
ttgattgagattgattttgagtat

SEQ ID NO: 1193 - DNA, 24 bp, Artificial Sequence
  Description of Artificial SequenceArtificially Synthesized DNA Sequence
tgaattgatgaatgaatgaagtat

SEQ ID NO: 1194 - DNA, 24 bp, Artificial Sequence
  Description of Artificial SequenceArtificially Synthesized DNA Sequence
gtaatgaagtatgtatgtaagtaa

SEQ ID NO: 1195 - DNA, 24 bp, Artificial Sequence
  Description of Artificial SequenceArtificially Synthesized DNA Sequence
tgatgatttgaatgaagattgatt

SEQ ID NO: 1196 - DNA, 24 bp, Artificial Sequence
  Description of Artificial SequenceArtificially Synthesized DNA Sequence
tgataaagtgataaaggattaaag

SEQ ID NO: 1197 - DNA, 24 bp, Artificial Sequence
  Description of Artificial SequenceArtificially Synthesized DNA Sequence
tgatttgagtatttgagattttga

SEQ ID NO: 1198 - DNA, 24 bp, Artificial Sequence
  Description of Artificial SequenceArtificially Synthesized DNA Sequence
tgtagtaagattgattaaaggtaa

SEQ ID NO: 1199 - DNA, 24 bp, Artificial Sequence
  Description of Artificial SequenceArtificially Synthesized DNA Sequence
gtataaaggattgattttgaaaag

SEQ ID NO: 1200 - DNA, 24 bp, Artificial Sequence
  Description of Artificial SequenceArtificially Synthesized DNA Sequence
gtatttgagtaagtaattgattga

SEQ ID NO: 1201 - DNA, 24 bp, Artificial Sequence
  Description of Artificial SequenceArtificially Synthesized DNA Sequence
gtaaaaagttgagtattgaaaaag

SEQ ID NO: 1202 - DNA, 24 bp, Artificial Sequence
  Description of Artificial SequenceArtificially Synthesized DNA Sequence
gatttgataaaggatttgtattga

SEQ ID NO: 1203 - DNA, 24 bp, Artificial Sequence
  Description of Artificial SequenceArtificially Synthesized DNA Sequence
gattgtattgaagtattgtaaaag

SEQ ID NO: 1204 - DNA, 24 bp, Artificial Sequence
  Description of Artificial SequenceArtificially Synthesized DNA Sequence
tgatgattttgatgaaaaagttga

SEQ ID NO: 1205 - DNA, 24 bp, Artificial Sequence
  Description of Artificial SequenceArtificially Synthesized DNA Sequence
tgatttgagattaaagaaaggatt

SEQ ID NO: 1206 - DNA, 24 bp, Artificial Sequence
  Description of Artificial SequenceArtificially Synthesized DNA Sequence
tgattgaattgagtaaaaaggatt

SEQ ID NO: 1207 - DNA, 24 bp, Artificial Sequence
  Description of Artificial SequenceArtificially Synthesized DNA Sequence
aaagtgtaaaaggatttgatgtat

SEQ ID NO: 1208 - DNA, 24 bp, Artificial Sequence
  Description of Artificial SequenceArtificially Synthesized DNA Sequence
aaaggtatttgagatttgattgaa

SEQ ID NO: 1209 - DNA, 24 bp, Artificial Sequence
  Description of Artificial SequenceArtificially Synthesized DNA Sequence
aaagttgagatttgaatgattgaa

SEQ ID NO: 1210 - DNA, 24 bp, Artificial Sequence
  Description of Artificial SequenceArtificially Synthesized DNA Sequence
tgtattgaaaaggtatgatttgaa

SEQ ID NO: 1211 - DNA, 24 bp, Artificial Sequence
  Description of Artificial SequenceArtificially Synthesized DNA Sequence
gtattgtattgaaaaggtaattga

SEQ ID NO: 1212 - DNA, 24 bp, Artificial Sequence
  Description of Artificial SequenceArtificially Synthesized DNA Sequence
ttgagtaatgataaagtgaagatt

SEQ ID NO: 1213 - DNA, 24 bp, Artificial Sequence
  Description of Artificial SequenceArtificially Synthesized DNA Sequence
tgaagatttgaagtaattgaaaag

SEQ ID NO: 1214 - DNA, 24 bp, Artificial Sequence
  Description of Artificial SequenceArtificially Synthesized DNA Sequence
tgaaaaagtgtagattttgagtaa

SEQ ID NO: 1215 - DNA, 24 bp, Artificial Sequence
  Description of Artificial SequenceArtificially Synthesized DNA Sequence
tgtatgaatgaagatttgattgta

SEQ ID NO: 1216 - DNA, 24 bp, Artificial Sequence
  Description of Artificial SequenceArtificially Synthesized DNA Sequence
aaagttgagtattgatttgaaaag

SEQ ID NO: 1217 - DNA, 24 bp, Artificial Sequence
  Description of Artificial SequenceArtificially Synthesized DNA Sequence
gatttgtagatttgtattgagatt

SEQ ID NO: 1218 - DNA, 24 bp, Artificial Sequence
  Description of Artificial SequenceArtificially Synthesized DNA Sequence
aaagaaaggatttgtagtaagatt

SEQ ID NO: 1219 - DNA, 24 bp, Artificial Sequence
  Description of Artificial SequenceArtificially Synthesized DNA Sequence
gtaaaaagaaaggtataaaggtaa

SEQ ID NO: 1220 - DNA, 24 bp, Artificial Sequence
  Description of Artificial SequenceArtificially Synthesized DNA Sequence
gattaaagttgattgaaaagtgaa

SEQ ID NO: 1221 - DNA, 24 bp, Artificial Sequence
  Description of Artificial SequenceArtificially Synthesized DNA Sequence
tgaaaaaggtaattgatgtatgaa

SEQ ID NO: 1222 - DNA, 24 bp, Artificial Sequence
  Description of Artificial SequenceArtificially Synthesized DNA Sequence
aaaggattaaagtgaagtaattga

SEQ ID NO: 1223 - DNA, 24 bp, Artificial Sequence
  Description of Artificial SequenceArtificially Synthesized DNA Sequence
atgaattggtatgtatatgaatga

SEQ ID NO: 1224 - DNA, 24 bp, Artificial Sequence
  Description of Artificial SequenceArtificially Synthesized DNA Sequence
tgaaatgaatgaatgatgaaattg

SEQ ID NO: 1225 - DNA, 24 bp, Artificial Sequence
  Description of Artificial SequenceArtificially Synthesized DNA Sequence
attgattgtgaatgaaatgaattg

SEQ ID NO: 1226 - DNA, 24 bp, Artificial Sequence
  Description of Artificial SequenceArtificially Synthesized DNA Sequence
attgaaagatgaaaagatgaaaag

SEQ ID NO: 1227 - DNA, 24 bp, Artificial Sequence
  Description of Artificial SequenceArtificially Synthesized DNA Sequence
attgttgaaaagtgtaatgattga

SEQ ID NO: 1228 - DNA, 24 bp, Artificial Sequence
  Description of Artificial SequenceArtificially Synthesized DNA Sequence
atgatgtaatgaaaagattgtgta

SEQ ID NO: 1229 - DNA, 24 bp, Artificial Sequence
  Description of Artificial SequenceArtificially Synthesized DNA Sequence
aaagattgaaagatgatgtaattg

SEQ ID NO: 1230 - DNA, 24 bp, Artificial Sequence
  Description of Artificial SequenceArtificially Synthesized DNA Sequence
attgatgagtatattgtgtagtaa

SEQ ID NO: 1231 - DNA, 24 bp, Artificial Sequence
  Description of Artificial SequenceArtificially Synthesized DNA Sequence
aaagattgtgtaattgatgatgaa

SEQ ID NO: 1232 - DNA, 24 bp, Artificial Sequence
  Description of Artificial SequenceArtificially Synthesized DNA Sequence
aaaggtatattgtgtaatgagtaa

SEQ ID NO: 1233 - DNA, 24 bp, Artificial Sequence
  Description of Artificial SequenceArtificially Synthesized DNA Sequence
tgtaatgagtattgtaattgaaag

SEQ ID NO: 1234 - DNA, 24 bp, Artificial Sequence
  Description of Artificial SequenceArtificially Synthesized DNA Sequence
gtataaagaaagattggtaaatga

SEQ ID NO: 1235 - DNA, 24 bp, Artificial Sequence
  Description of Artificial SequenceArtificially Synthesized DNA Sequence
ttgagtaattgaattgtgaaatga

SEQ ID NO: 1236 - DNA, 24 bp, Artificial Sequence
  Description of Artificial SequenceArtificially Synthesized DNA Sequence
tgtattgaatgaattgttgatgta

SEQ ID NO: 1237 - DNA, 24 bp, Artificial Sequence
  Description of Artificial SequenceArtificially Synthesized DNA Sequence
tgtaattggtaaatgagtaaaaag

SEQ ID NO: 1238 - DNA, 24 bp, Artificial Sequence
  Description of Artificial SequenceArtificially Synthesized DNA Sequence
tgaatgaaattgatgagtataaag

SEQ ID NO: 1239 - DNA, 24 bp, Artificial Sequence
  Description of Artificial SequenceArtificially Synthesized DNA Sequence
gtaagtaaattgaaagattgatga

SEQ ID NO: 1240 - DNA, 24 bp, Artificial Sequence
  Description of Artificial SequenceArtificially Synthesized DNA Sequence
gtaaatgatgatattggtatattg

SEQ ID NO: 1241 - DNA, 24 bp, Artificial Sequence
  Description of Artificial SequenceArtificially Synthesized DNA Sequence
attgttgatgattgattgaaatga

SEQ ID NO: 1242 - DNA, 24 bp, Artificial Sequence
  Description of Artificial SequenceArtificially Synthesized DNA Sequence
attgtgaagtataaagatgattga

SEQ ID NO: 1243 - DNA, 24 bp, Artificial Sequence
  Description of Artificial SequenceArtificially Synthesized DNA Sequence
atgaaaagttgagtaaattgtgat

SEQ ID NO: 1244 - DNA, 24 bp, Artificial Sequence
  Description of Artificial SequenceArtificially Synthesized DNA Sequence
atgaattgaaagtgattgaaaaag

SEQ ID NO: 1245 - DNA, 24 bp, Artificial Sequence
  Description of Artificial SequenceArtificially Synthesized DNA Sequence
gtaaattgatgaaaagttgatgat

SEQ ID NO: 1246 - DNA, 24 bp, Artificial Sequence
  Description of Artificial SequenceArtificially Synthesized DNA Sequence
aaagtgatgtatatgagtaaattg

SEQ ID NO: 1247 - DNA, 24 bp, Artificial Sequence
  Description of Artificial SequenceArtificially Synthesized DNA Sequence
gtaatgataaagatgatgatattg

SEQ ID NO: 1248 - DNA, 24 bp, Artificial Sequence
  Description of Artificial SequenceArtificially Synthesized DNA Sequence
ttgaaaagattggtaatgatatga

SEQ ID NO: 1249 - DNA, 24 bp, Artificial Sequence
  Description of Artificial SequenceArtificially Synthesized DNA Sequence
aaagtgaaaaagattgattgatga

SEQ ID NO: 1250 - DNA, 24 bp, Artificial Sequence
  Description of Artificial SequenceArtificially Synthesized DNA Sequence
attgatgagattgattattgtgta

SEQ ID NO: 1251 - DNA, 24 bp, Artificial Sequence
  Description of Artificial SequenceArtificially Synthesized DNA Sequence
atgagattattggatttgtagatt

SEQ ID NO: 1252 - DNA, 24 bp, Artificial Sequence
  Description of Artificial SequenceArtificially Synthesized DNA Sequence
tgaagattatgaattggtaagatt

SEQ ID NO: 1253 - DNA, 24 bp, Artificial Sequence
  Description of Artificial SequenceArtificially Synthesized DNA Sequence
attggattatgagattatgattga

SEQ ID NO: 1254 - DNA, 24 bp, Artificial Sequence
  Description of Artificial SequenceArtificially Synthesized DNA Sequence
attgttgaattggattaaagatga

SEQ ID NO: 1255 - DNA, 24 bp, Artificial Sequence
  Description of Artificial SequenceArtificially Synthesized DNA Sequence
aaagatgagtaagtaaattggatt

SEQ ID NO: 1256 - DNA, 24 bp, Artificial Sequence
  Description of Artificial SequenceArtificially Synthesized DNA Sequence
aaaggtaagattattgatgaaaag

SEQ ID NO: 1257 - DNA, 24 bp, Artificial Sequence
  Description of Artificial SequenceArtificially Synthesized DNA Sequence
attgatgagattaaagttgaattg

SEQ ID NO: 1258 - DNA, 24 bp, Artificial Sequence
  Description of Artificial SequenceArtificially Synthesized DNA Sequence
gattattggattatgaaaaggatt

SEQ ID NO: 1259 - DNA, 24 bp, Artificial Sequence
  Description of Artificial SequenceArtificially Synthesized DNA Sequence
gatttgtaattgttgagtaaatga

SEQ ID NO: 1260 - DNA, 24 bp, Artificial Sequence
  Description of Artificial SequenceArtificially Synthesized DNA Sequence
aaagaaagattgttgagattatga

SEQ ID NO: 1261 - DNA, 24 bp, Artificial Sequence
  Description of Artificial SequenceArtificially Synthesized DNA Sequence
gtataaaggattttgaattgatga

SEQ ID NO: 1262 - DNA, 24 bp, Artificial Sequence
  Description of Artificial SequenceArtificially Synthesized DNA Sequence
ttgagattgtaaatgaattgttga

SEQ ID NO: 1263 - DNA, 24 bp, Artificial Sequence
  Description of Artificial SequenceArtificially Synthesized DNA Sequence
gtatattgattgtgtaatgaaaag

SEQ ID NO: 1264 - DNA, 24 bp, Artificial Sequence
  Description of Artificial SequenceArtificially Synthesized DNA Sequence
tgatatgaattggattattggtat

SEQ ID NO: 1265 - DNA, 24 bp, Artificial Sequence
  Description of Artificial SequenceArtificially Synthesized DNA Sequence
atgaatgatgaatgatgattattg

SEQ ID NO: 1266 - DNA, 24 bp, Artificial Sequence
  Description of Artificial SequenceArtificially Synthesized DNA Sequence
atgaattgattggattgtaatgat

SEQ ID NO: 1267 - DNA, 24 bp, Artificial Sequence
  Description of Artificial SequenceArtificially Synthesized DNA Sequence
gattgtaattgagtaaattgatga

SEQ ID NO: 1268 - DNA, 24 bp, Artificial Sequence
  Description of Artificial SequenceArtificially Synthesized DNA Sequence
gattattggattaaaggtaaatga

SEQ ID NO: 1269 - DNA, 24 bp, Artificial Sequence
  Description of Artificial SequenceArtificially Synthesized DNA Sequence
attgttgaattgatgagatttgat

SEQ ID NO: 1270 - DNA, 24 bp, Artificial Sequence
  Description of Artificial SequenceArtificially Synthesized DNA Sequence
gattatgagtaaattgattgtgat

SEQ ID NO: 1271 - DNA, 24 bp, Artificial Sequence
  Description of Artificial SequenceArtificially Synthesized DNA Sequence
gattattgttgatgaatgatattg

SEQ ID NO: 1272 - DNA, 24 bp, Artificial Sequence
  Description of Artificial SequenceArtificially Synthesized DNA Sequence
tgtaaaagattgaaaggtatgatt

SEQ ID NO: 1273 - DNA, 24 bp, Artificial Sequence
  Description of Artificial SequenceArtificially Synthesized DNA Sequence
gtatttagatgagtttgttagatt

SEQ ID NO: 1274 - DNA, 24 bp, Artificial Sequence
  Description of Artificial SequenceArtificially Synthesized DNA Sequence
tgaagttatgtaatagaaagtgat

SEQ ID NO: 1275 - DNA, 24 bp, Artificial Sequence
  Description of Artificial SequenceArtificially Synthesized DNA Sequence
gtatgtattgtatgtagttaattg

SEQ ID NO: 1276 - DNA, 24 bp, Artificial Sequence
  Description of Artificial SequenceArtificially Synthesized DNA Sequence
tgatatagatagttagatagatag

SEQ ID NO: 1277 - DNA, 24 bp, Artificial Sequence
  Description of Artificial SequenceArtificially Synthesized DNA Sequence
atgatgatgtattgtagttatgaa

SEQ ID NO: 1278 - DNA, 24 bp, Artificial Sequence
  Description of Artificial SequenceArtificially Synthesized DNA Sequence
ttagtgaatgtattagttgatgta

SEQ ID NO: 1279 - DNA, 24 bp, Artificial Sequence
  Description of Artificial SequenceArtificially Synthesized DNA Sequence
gttagttagattattgttagttag

SEQ ID NO: 1280 - DNA, 24 bp, Artificial Sequence
  Description of Artificial SequenceArtificially Synthesized DNA Sequence
gttaattgtgtagtttgttattga

SEQ ID NO: 1281 - DNA, 24 bp, Artificial Sequence
  Description of Artificial SequenceArtificially Synthesized DNA Sequence
gttatgaaatagtgatattgttag

SEQ ID NO: 1282 - DNA, 24 bp, Artificial Sequence
  Description of Artificial SequenceArtificially Synthesized DNA Sequence
attgttagaaagtgtagattaaag

SEQ ID NO: 1283 - DNA, 24 bp, Artificial Sequence
  Description of Artificial SequenceArtificially Synthesized DNA Sequence
atgagtatgttattagtgtatgta

SEQ ID NO: 1284 - DNA, 24 bp, Artificial Sequence
  Description of Artificial SequenceArtificially Synthesized DNA Sequence
tgtaatagtgaagttagattgtat

SEQ ID NO: 1285 - DNA, 24 bp, Artificial Sequence
  Description of Artificial SequenceArtificially Synthesized DNA Sequence
attgatagatgattagttagttga

SEQ ID NO: 1286 - DNA, 24 bp, Artificial Sequence
  Description of Artificial SequenceArtificially Synthesized DNA Sequence
atgagtttgtttatgagattaaag

SEQ ID NO: 1287 - DNA, 24 bp, Artificial Sequence
  Description of Artificial SequenceArtificially Synthesized DNA Sequence
tgatgtttgattatgatgtagtat

SEQ ID NO: 1288 - DNA, 24 bp, Artificial Sequence
  Description of Artificial SequenceArtificially Synthesized DNA Sequence
atgagttagttatgaattagatga

SEQ ID NO: 1289 - DNA, 24 bp, Artificial Sequence
  Description of Artificial SequenceArtificially Synthesized DNA Sequence
attgttagtgatgttagtaattag

SEQ ID NO: 1290 - DNA, 24 bp, Artificial Sequence
  Description of Artificial SequenceArtificially Synthesized DNA Sequence
tgatgtaagtattgatgttagttt

SEQ ID NO: 1291 - DNA, 24 bp, Artificial Sequence
  Description of Artificial SequenceArtificially Synthesized DNA Sequence
gattgtaaatagaaagtgaagtaa

SEQ ID NO: 1292 - DNA, 24 bp, Artificial Sequence
  Description of Artificial SequenceArtificially Synthesized DNA Sequence
attgtgtatgaagtattgtatgat

SEQ ID NO: 1293 - DNA, 24 bp, Artificial Sequence
  Description of Artificial SequenceArtificially Synthesized DNA Sequence
atagtgatgttatgaagattgtta

SEQ ID NO: 1294 - DNA, 24 bp, Artificial Sequence
  Description of Artificial SequenceArtificially Synthesized DNA Sequence
ttagatgaattgtgaagtatttag

SEQ ID NO: 1295 - DNA, 24 bp, Artificial Sequence
  Description of Artificial SequenceArtificially Synthesized DNA Sequence
gtaagttatgattgatgttatgaa

SEQ ID NO: 1296 - DNA, 24 bp, Artificial Sequence
  Description of Artificial SequenceArtificially Synthesized DNA Sequence
gtattgatgtttaaagtgtaatag

SEQ ID NO: 1297 - DNA, 24 bp, Artificial Sequence
  Description of Artificial SequenceArtificially Synthesized DNA Sequence
gattgtaagtaagattgtatattg

SEQ ID NO: 1298 - DNA, 24 bp, Artificial Sequence
  Description of Artificial SequenceArtificially Synthesized DNA Sequence
gtttgtatttagatgaatagaaag

SEQ ID NO: 1299 - DNA, 24 bp, Artificial Sequence
  Description of Artificial SequenceArtificially Synthesized DNA Sequence
gtttgatttgtaatagtgattgta

SEQ ID NO: 1300 - DNA, 24 bp, Artificial Sequence
  Description of Artificial SequenceArtificially Synthesized DNA Sequence
tgtatgtagtatttagaaagatga

SEQ ID NO: 1301 - DNA, 24 bp, Artificial Sequence
  Description of Artificial SequenceArtificially Synthesized DNA Sequence
atgaattgtgataaagaaagttag

SEQ ID NO: 1302 - DNA, 24 bp, Artificial Sequence
  Description of Artificial SequenceArtificially Synthesized DNA Sequence
ttagtgtagtaagtttaaagtgta

SEQ ID NO: 1303 - DNA, 24 bp, Artificial Sequence
  Description of Artificial SequenceArtificially Synthesized DNA Sequence
gtatgattgtttgtaattagtgat

SEQ ID NO: 1304 - DNA, 24 bp, Artificial Sequence
  Description of Artificial SequenceArtificially Synthesized DNA Sequence
gtttaaagttagttgagttagtat

SEQ ID NO: 1305 - DNA, 24 bp, Artificial Sequence
  Description of Artificial SequenceArtificially Synthesized DNA Sequence
atagtgtatgtagattatgagatt

SEQ ID NO: 1306 - DNA, 24 bp, Artificial Sequence
  Description of Artificial SequenceArtificially Synthesized DNA Sequence
ttgaatgattagttgagtatgatt

SEQ ID NO: 1307 - DNA, 24 bp, Artificial Sequence
  Description of Artificial SequenceArtificially Synthesized DNA Sequence
gtatgtaagttagtatgatttgaa

SEQ ID NO: 1308 - DNA, 24 bp, Artificial Sequence
  Description of Artificial SequenceArtificially Synthesized DNA Sequence
tgtagtatattgttgaattgtgat

SEQ ID NO: 1309 - DNA, 24 bp, Artificial Sequence
  Description of Artificial SequenceArtificially Synthesized DNA Sequence
atagtgattgtatgtatgataaag

SEQ ID NO: 1310 - DNA, 24 bp, Artificial Sequence
  Description of Artificial SequenceArtificially Synthesized DNA Sequence
ttagtgattgatgtatattgaaag

SEQ ID NO: 1311 - DNA, 24 bp, Artificial Sequence
  Description of Artificial SequenceArtificially Synthesized DNA Sequence
gtaagattatgagttatgatgtaa

SEQ ID NO: 1312 - DNA, 24 bp, Artificial Sequence
  Description of Artificial SequenceArtificially Synthesized DNA Sequence
gttatgaaattgttagtgtagatt

SEQ ID NO: 1313 - DNA, 24 bp, Artificial Sequence
  Description of Artificial SequenceArtificially Synthesized DNA Sequence
gttagatttgtagtttaaagatag

SEQ ID NO: 1314 - DNA, 24 bp, Artificial Sequence
  Description of Artificial SequenceArtificially Synthesized DNA Sequence
ttagtgattgaaatgatgtagatt

SEQ ID NO: 1315 - DNA, 24 bp, Artificial Sequence
  Description of Artificial SequenceArtificially Synthesized DNA Sequence
aaagtgtagttattagttagttag

SEQ ID NO: 1316 - DNA, 24 bp, Artificial Sequence
  Description of Artificial SequenceArtificially Synthesized DNA Sequence
aaagaaagtgtatgatgttattag

SEQ ID NO: 1317 - DNA, 24 bp, Artificial Sequence
  Description of Artificial SequenceArtificially Synthesized DNA Sequence
gattgtatattgtgtatgatgatt

SEQ ID NO: 1318 - DNA, 24 bp, Artificial Sequence
  Description of Artificial SequenceArtificially Synthesized DNA Sequence
ttgagattgttatgatatgagtat

SEQ ID NO: 1319 - DNA, 24 bp, Artificial Sequence
  Description of Artificial SequenceArtificially Synthesized DNA Sequence
atgagtatgattgttatgatgttt

SEQ ID NO: 1320 - DNA, 24 bp, Artificial Sequence
  Description of Artificial SequenceArtificially Synthesized DNA Sequence
tgatttagtgaaattgtgtattag

SEQ ID NO: 1321 - DNA, 24 bp, Artificial Sequence
  Description of Artificial SequenceArtificially Synthesized DNA Sequence
tgaatgtatgtagtatgtttgtta

SEQ ID NO: 1322 - DNA, 24 bp, Artificial Sequence
  Description of Artificial SequenceArtificially Synthesized DNA Sequence
gttagtattgatgattatgagtta

SEQ ID NO: 1323 - DNA, 24 bp, Artificial Sequence
  Description of Artificial SequenceArtificially Synthesized DNA Sequence
gtatattgtgatttagttgagatt

SEQ ID NO: 1324 - DNA, 24 bp, Artificial Sequence
  Description of Artificial SequenceArtificially Synthesized DNA Sequence
gttagtttaaagttgagattgttt

SEQ ID NO: 1325 - DNA, 24 bp, Artificial Sequence
  Description of Artificial SequenceArtificially Synthesized DNA Sequence
gtatattgttagatgagatttgta

SEQ ID NO: 1326 - DNA, 24 bp, Artificial Sequence
  Description of Artificial SequenceArtificially Synthesized DNA Sequence
tgatgtatgttagtttatgaatga

SEQ ID NO: 1327 - DNA, 24 bp, Artificial Sequence
  Description of Artificial SequenceArtificially Synthesized DNA Sequence
tgtagtatgtaatgtagtatttga

SEQ ID NO: 1328 - DNA, 24 bp, Artificial Sequence
  Description of Artificial SequenceArtificially Synthesized DNA Sequence
atgagttatgtattgagttagtat

SEQ ID NO: 1329 - DNA, 24 bp, Artificial Sequence
  Description of Artificial SequenceArtificially Synthesized DNA Sequence
tgtatgatgattatagttgagtaa

SEQ ID NO: 1330 - DNA, 24 bp, Artificial Sequence
  Description of Artificial SequenceArtificially Synthesized DNA Sequence
attgatgaatgagtttgtataaag

SEQ ID NO: 1331 - DNA, 24 bp, Artificial Sequence
  Description of Artificial SequenceArtificially Synthesized DNA Sequence
ttgagtttatgattagaaagaaag

SEQ ID NO: 1332 - DNA, 24 bp, Artificial Sequence
  Description of Artificial SequenceArtificially Synthesized DNA Sequence
tgatattgatgagttagtattgaa

SEQ ID NO: 1333 - DNA, 24 bp, Artificial Sequence
  Description of Artificial SequenceArtificially Synthesized DNA Sequence
atagaaagtgaaatgagtatgtta

SEQ ID NO: 1334 - DNA, 24 bp, Artificial Sequence
  Description of Artificial SequenceArtificially Synthesized DNA Sequence
ttgatgtagatttgatgtatatag

SEQ ID NO: 1335 - DNA, 24 bp, Artificial Sequence
  Description of Artificial SequenceArtificially Synthesized DNA Sequence
ttgagattatagtgtagtttatag

SEQ ID NO: 1336 - DNA, 24 bp, Artificial Sequence
  Description of Artificial SequenceArtificially Synthesized DNA Sequence
tgatgttagattgtttgattattg

SEQ ID NO: 1337 - DNA, 24 bp, Artificial Sequence
  Description of Artificial SequenceArtificially Synthesized DNA Sequence
tgtattagatagtgatttgaatga

SEQ ID NO: 1338 - DNA, 24 bp, Artificial Sequence
  Description of Artificial SequenceArtificially Synthesized DNA Sequence
gattatgatgaatgtagtatgtaa

SEQ ID NO: 1339 - DNA, 24 bp, Artificial Sequence
  Description of Artificial SequenceArtificially Synthesized DNA Sequence
tgaatgattgatatgaatagtgta

SEQ ID NO: 1340 - DNA, 24 bp, Artificial Sequence
  Description of Artificial SequenceArtificially Synthesized DNA Sequence
gtaatgatttagtgtattgagttt

SEQ ID NO: 1341 - DNA, 24 bp, Artificial Sequence
  Description of Artificial SequenceArtificially Synthesized DNA Sequence
tgtagtaatgatttgatgataaag

SEQ ID NO: 1342 - DNA, 24 bp, Artificial Sequence
  Description of Artificial SequenceArtificially Synthesized DNA Sequence
tgaagattgttattagtgatattg

SEQ ID NO: 1343 - DNA, 24 bp, Artificial Sequence
  Description of Artificial SequenceArtificially Synthesized DNA Sequence
gtatttgaatgatgtaatagtgta

SEQ ID NO: 1344 - DNA, 24 bp, Artificial Sequence
  Description of Artificial SequenceArtificially Synthesized DNA Sequence
gtatatgatgtattagattgaaag

SEQ ID NO: 1345 - DNA, 24 bp, Artificial Sequence
  Description of Artificial SequenceArtificially Synthesized DNA Sequence
aaagttagattgaaagtgataaag

SEQ ID NO: 1346 - DNA, 24 bp, Artificial Sequence
  Description of Artificial SequenceArtificially Synthesized DNA Sequence
gtaagatgttgatatagaagatta

SEQ ID NO: 1347 - DNA, 24 bp, Artificial Sequence
  Description of Artificial SequenceArtificially Synthesized DNA Sequence
taatatgagatgaaagtgaattag

SEQ ID NO: 1348 - DNA, 24 bp, Artificial Sequence
  Description of Artificial SequenceArtificially Synthesized DNA Sequence
ttagtgaagaagtatagtttattg

SEQ ID NO: 1349 - DNA, 24 bp, Artificial Sequence
  Description of Artificial SequenceArtificially Synthesized DNA Sequence
gtagttgagaagatagtaattaat

SEQ ID NO: 1350 - DNA, 24 bp, Artificial Sequence
  Description of Artificial SequenceArtificially Synthesized DNA Sequence
atgagatgatatttgagaagtaat

SEQ ID NO: 1351 - DNA, 24 bp, Artificial Sequence
  Description of Artificial SequenceArtificially Synthesized DNA Sequence
gatgtgaagaagatgaatatatat

SEQ ID NO: 1352 - DNA, 24 bp, Artificial Sequence
  Description of Artificial SequenceArtificially Synthesized DNA Sequence
aaagtatagtaagatgtatagtag

SEQ ID NO: 1353 - DNA, 24 bp, Artificial Sequence
  Description of Artificial SequenceArtificially Synthesized DNA Sequence
gaagtaatatgagtagttgaatat

SEQ ID NO: 1354 - DNA, 24 bp, Artificial Sequence
  Description of Artificial SequenceArtificially Synthesized DNA Sequence
ttgataatgtttgtttgtttgtag

SEQ ID NO: 1355 - DNA, 24 bp, Artificial Sequence
  Description of Artificial SequenceArtificially Synthesized DNA Sequence
tgaagaagaaagtataatgatgaa

SEQ ID NO: 1356 - DNA, 24 bp, Artificial Sequence
  Description of Artificial SequenceArtificially Synthesized DNA Sequence
gtagattagtttgaagtgaataat

SEQ ID NO: 1357 - DNA, 24 bp, Artificial Sequence
  Description of Artificial SequenceArtificially Synthesized DNA Sequence
tatagtagtgaagatgatatatga

SEQ ID NO: 1358 - DNA, 24 bp, Artificial Sequence
  Description of Artificial SequenceArtificially Synthesized DNA Sequence
tataatgagttgttagatatgttg

SEQ ID NO: 1359 - DNA, 24 bp, Artificial Sequence
  Description of Artificial SequenceArtificially Synthesized DNA Sequence
gttgtgaaattagatgtgaaatat

SEQ ID NO: 1360 - DNA, 24 bp, Artificial Sequence
  Description of Artificial SequenceArtificially Synthesized DNA Sequence
taatgttgtgaataatgtagaaag

SEQ ID NO: 1361 - DNA, 24 bp, Artificial Sequence
  Description of Artificial SequenceArtificially Synthesized DNA Sequence
gtttatagtgaaatatgaagatag

SEQ ID NO: 1362 - DNA, 24 bp, Artificial Sequence
  Description of Artificial SequenceArtificially Synthesized DNA Sequence
attatgaagtaagttaatgagaag

SEQ ID NO: 1363 - DNA, 24 bp, Artificial Sequence
  Description of Artificial SequenceArtificially Synthesized DNA Sequence
gatgaaagtaatgtttattgtgaa

SEQ ID NO: 1364 - DNA, 24 bp, Artificial Sequence
  Description of Artificial SequenceArtificially Synthesized DNA Sequence
attattgagatgtgaagtttgttt

SEQ ID NO: 1365 - DNA, 24 bp, Artificial Sequence
  Description of Artificial SequenceArtificially Synthesized DNA Sequence
tgtagaagatgagatgtataatta

SEQ ID NO: 1366 - DNA, 24 bp, Artificial Sequence
  Description of Artificial SequenceArtificially Synthesized DNA Sequence
taatttgagttgtgtatatagtag

SEQ ID NO: 1367 - DNA, 24 bp, Artificial Sequence
  Description of Artificial SequenceArtificially Synthesized DNA Sequence
tgatattagtaagaagttgaatag

SEQ ID NO: 1368 - DNA, 24 bp, Artificial Sequence
  Description of Artificial SequenceArtificially Synthesized DNA Sequence
gttagttattgagaagtgtatata

SEQ ID NO: 1369 - DNA, 24 bp, Artificial Sequence
  Description of Artificial SequenceArtificially Synthesized DNA Sequence
gtagtaatgttaatgaattagtag

SEQ ID NO: 1370 - DNA, 24 bp, Artificial Sequence
  Description of Artificial SequenceArtificially Synthesized DNA Sequence
gtttgtttgatgtgattgaataat

SEQ ID NO: 1371 - DNA, 24 bp, Artificial Sequence
  Description of Artificial SequenceArtificially Synthesized DNA Sequence
gtaagtagtaatttgaatatgtag

SEQ ID NO: 1372 - DNA, 24 bp, Artificial Sequence
  Description of Artificial SequenceArtificially Synthesized DNA Sequence
gtttgaagatatgtttgaagtata

SEQ ID NO: 1373 - DNA, 24 bp, Artificial Sequence
  Description of Artificial SequenceArtificially Synthesized DNA Sequence
atgataattgaagatgtaatgttg

SEQ ID NO: 1374 - DNA, 24 bp, Artificial Sequence
  Description of Artificial SequenceArtificially Synthesized DNA Sequence
gtagatagtatagttgtaatgtta

SEQ ID NO: 1375 - DNA, 24 bp, Artificial Sequence
  Description of Artificial SequenceArtificially Synthesized DNA Sequence
gatgtgaatgtaatatgtttatag

SEQ ID NO: 1376 - DNA, 24 bp, Artificial Sequence
  Description of Artificial SequenceArtificially Synthesized DNA Sequence
tgaaattagtttgtaagatgtgta

SEQ ID NO: 1377 - DNA, 24 bp, Artificial Sequence
  Description of Artificial SequenceArtificially Synthesized DNA Sequence
tgtagtataaagtatatgaagtag

SEQ ID NO: 1378 - DNA, 24 bp, Artificial Sequence
  Description of Artificial SequenceArtificially Synthesized DNA Sequence
atatgttgttgagttgatagtata

SEQ ID NO: 1379 - DNA, 24 bp, Artificial Sequence
  Description of Artificial SequenceArtificially Synthesized DNA Sequence
attattgagtagaaagatagaaag

SEQ ID NO: 1380 - DNA, 24 bp, Artificial Sequence
  Description of Artificial SequenceArtificially Synthesized DNA Sequence
gttgttgaatattgaatatagttg

SEQ ID NO: 1381 - DNA, 24 bp, Artificial Sequence
  Description of Artificial SequenceArtificially Synthesized DNA Sequence
atgagaagttagtaatgtaaatag

SEQ ID NO: 1382 - DNA, 24 bp, Artificial Sequence
  Description of Artificial SequenceArtificially Synthesized DNA Sequence
tgaaatgagaagattaatgagttt

SEQ ID NO: 1383 - DNA, 24 bp, Artificial Sequence
  Description of Artificial SequenceArtificially Synthesized DNA Sequence
atgtttagtgaaaagttagtattg

SEQ ID NO: 1384 - DNA, 24 bp, Artificial Sequence
  Description of Artificial SequenceArtificially Synthesized DNA Sequence
atgttagtgaatagtatagtattg